(19) United States
(12) Patent Application Publication
(10) Pub. No.: US 2024/0335286 A1
(43) Pub. Date: Oct. 10, 2024

(54) HEART VALVE SEALING DEVICES AND DELIVERY DEVICES THEREFOR

(71) Applicant: EDWARDS LIFESCIENCES CORPORATION, Irvine, CA (US)

(72) Inventors: Aviv Galon, Tel Aviv (IL); Eran Hoffer, Yahud (IL); Carmel Peleg, Pardes Hana-Karkur (IL); Eric Michael Oberwise, Newport Beach, CA (US); Bezalel Haberman-Browns, Jerusalem (IL); Sarit Avivi, Givat Shmuel (IL); Haim Brauon, Beit Dagan (IL); Ido Halabi, Givatayim (IL); Jeffrey Michael Koslosky, Coto De Caza, CA (US); Meir Abucasis, Caesarea (IL); Waina Michelle Chu, Tustin, CA (US)

(21) Appl. No.: 18/737,888

(22) Filed: Jun. 7, 2024

Related U.S. Application Data
(63) Continuation of application No. PCT/US2022/051232, filed on Nov. 29, 2022.
(60) Provisional application No. 63/287,907, filed on Dec. 9, 2021.

(51) Int. Cl. A61F 2/24

(52) U.S. Cl. CPC A61F 2/2463; A61F 2/246; A61F 2/2466; A61F 2220/0091

(57) ABSTRACT

A device or implant is configured to be positioned within a native heart valve to allow the native heart valve to form a more effective seal. One or more portions of device or implant can expand and contract. For example, one or more portions of the device or implant can narrow during delivery and expand on implantation on the native heart valve. Delivery systems are configured to narrow and expand the one or more portions of the device or implant.

### RELATED APPLICATIONS

The present application is continuation of Patent Cooperation Treaty Application no. PCT/US2022/051232, filed on Nov. 29, 2022, titled “Heart Valve Sealing Devices and Delivery Devices Therefor”, which claims the benefit of U.S. Provisional Application No. 63/287,907, filed on Dec. 9, 2021, titled “Heart Valve Repair Devices and Delivery Devices Therefor”, which are incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, an example heart valve repair system includes a device (e.g., a repair device, an implantable device, etc.) with a proximal head and an adjustable member attached to the proximal head. In some implementations, a delivery system for the device includes a distal gripper and a coupling assembly for attaching the device to the distal gripper.

In some implementations, the delivery system includes a retention member extending through the delivery system and into the coupling assembly to secure the distal gripper in the coupled condition.

In some implementations, the coupling assembly is configured to transmits rotational movement of the distal gripper to the adjustable member via the proximal head when the coupling assembly is in a coupled condition.

In some implementations, the retention member extends or is extendable through the coupling assembly, in some cases to engage the adjustable member.

In some implementations, the proximal head is releasably attached to the adjustable member.

In some implementations, the proximal head includes a plurality of engagement protrusions and the distal gripper comprises a plurality of engagement protrusions for intermeshing with the engagement protrusions of the proximal head.

In some implementations, the retention member comprises a threaded distal end and the proximal head comprises a threaded opening for receiving the threaded distal end. In some implementations, the system is configured such that threading the threaded distal end of the retention member into the threaded opening of the proximal head retains the coupling assembly in the coupled condition.

In some implementations, the distal gripper has a plurality of flexible fingers, and each flexible finger includes an engaging portion. The proximal head has an opening for receiving the flexible fingers and windows for receiving the engaging portions of the flexible fingers.

In some implementations, extending the retention member between the flexible fingers prohibits the engaging portions of the flexible fingers from disengaging from the windows.

In some implementations, the flexible fingers are biased inward so that removal of the retention member allows the flexible fingers to move inward so that the engaging portions disengage from the windows.

In some implementations, the distal gripper has a pivoting engaging member and the proximal head comprises a window for receiving the pivoting engaging member.

In some implementations, the system is configured such that extending the retention member into the distal gripper engages the pivoting engaging member to cause the pivoting engaging member to pivot outwards to engage the window of the proximal head.

In some implementations, the pivoting engaging member is biased to pivot in an inward direction to facilitate disengagement from the window of the proximal head.

In some implementations, the distal gripper includes a sliding engaging member, and the proximal head has a window for receiving the sliding engaging member.

In some implementations, the system is configured such that extending the retention member into the distal gripper engages the sliding engaging member to cause the sliding engaging member to slide outwards to engage the window of the proximal head.

In some implementations, the sliding engaging member is biased to slide in an inward direction to facilitate disengagement from the window of the proximal head.

In some implementations, the distal gripper has a deployable engaging member attached to the distal gripper via a linkage and the proximal head includes a recess for receiving the deployable engaging member.

In some implementations, the system is configured such that distal movement of the distal gripper causes the deployable engaging member to move into the recess of the proximal head.

In some implementations, the system is configured such that extending the retention member between the distal gripper and the deployable engaging member secures the deployable engaging member in the recess of the proximal head.

In some implementations, the distal gripper includes a slot, and the proximal head has a laterally extending pin for engaging the slot of the distal gripper. In some implementations, the system is configured such that extending the retention member within the distal gripper secures the laterally extending pin within the slot.

In some implementations, the distal gripper has a keyed end having an engaging member and the proximal head includes an opening configured to receive the keyed end of the distal gripper only when the distal gripper is offset axially from the proximal head.

In some implementations, the system is configured such that extending the retention member through the distal gripper and the proximal head secures the engaging member of the keyed end in a window of the proximal head.

In some implementations, the distal gripper includes a window, and the proximal head includes an engaging protrusion configured to extend into and be received by the window of the distal gripper.

In some implementations, the system is configured such that extending the retention member through the distal gripper and the proximal head secures the engaging member of the proximal head in the window of the distal gripper.

In some implementations, the distal gripper includes a plurality of flexible fingers, each flexible finger having a window. The proximal head has an opening for receiving the flexible fingers and engaging portions for engaging the windows of the flexible fingers.

In some implementations, the system is configured such that extending the retention member between the flexible fingers prohibits the windows of the flexible fingers from disengaging from the engaging portions.

In some implementations, wherein the flexible fingers are biased inward so that removal of the retention member allows the flexible fingers to move inward so that the windows disengage from the engaging portions.

In some implementations, the distal gripper has a deployable engaging member that is attached to the distal gripper by a linkage and the proximal head includes an opening for receiving the distal gripper and a recess within the opening for receiving the deployable engaging member.

In some implementations, wherein when the deployable engaging member contacts a bottom of the opening in the proximal head, the deployable engaging member moves radially outward and into the recess.

In some implementations, wherein the retention member extends between the deployable engaging member and the distal gripper to prohibit the distal gripper from moving from a deployed condition to a retracted condition.

In some implementations, the proximal head includes a laterally oriented pin that is received in a slot of the distal gripper. An axial opening of the distal gripper is configured to receive the retention member so that the retention member extends beyond the laterally oriented pin of the proximal head to retain the laterally oriented pin in the slot of the distal gripper.

In some implementations, the distal gripper includes a tapered opening for guiding the retention member toward an open end of the slot.

In some implementations, the distal gripper and the proximal head are axially aligned when the laterally oriented pin is retained in the slot by the retention member.

In some implementations, an actuation assembly or an actuation mechanism for a device (e.g., a repair device, an implantable device, etc.) includes an outer tube with a plurality of openings, a latch tube attached to an adjustable member, and an actuation member having a threaded portion that can be threadably attached to a threaded opening of the latch tube and a tapered distal end.

In some implementations, when the threaded portion of the actuation member is threaded into the threaded opening of the latch tube, the tapered distal end engages the latch member and retains the latch member in an unlatched condition so that the latch tube can be moved to a desired position in the outer tube.

In some implementations, the actuation member can be unthreaded from the threaded opening of the latch tube to disengage the tapered distal end from the latch member to facilitate movement of the latch member to the latched condition wherein the latch member engages one of the plurality of openings of the outer tube.

In some implementations, the latch member is laser cut from the latch tube.

In some implementations, the latch member is biased toward the latched condition.

In some implementations, the latch tube also has an orientation tab that extends through an orientation slot of the outer tube to prohibit relative rotation of the latch tube and outer tube.

In some implementations, an actuation assembly or an actuation mechanism for a device (e.g., a repair device, an implantable device, etc.) has an outer tube with a plurality of openings, a latch tube attached to an adjustable member, an actuation tube having a distal end that forms one half of a coupling connection and is configured to releasably couple to a proximal end of the latch tube that forms the other half of the coupling connection.

In some implementations, a securing member extends through the actuation tube and latch tube to prohibit the decoupling of the coupling connection when the actuation tube and latch tube are in a coupled condition, the securing member having a tapered distal end.

In some implementations, when the securing member is inserted into the latch tube, the tapered distal end engages the latch member and retains the latch member in an unlatched condition so that the latch tube can be moved to a desired position in the outer tube.

In some implementations, the securing member can be withdrawn from the latch tube to disengage the tapered distal end from the latch member to facilitate movement of the latch member to the latched condition wherein the latch member engages one of the plurality of openings of the outer tube. In some implementations, the securing member can also be withdrawn from the coupling connection to allow the actuation tube to disengage from the latch tube.

In some implementations, the latch member is laser cut from the latch tube.

In some implementations, the latch member is biased toward the latched condition.

In some implementations, the latch tube further comprises an orientation pin that extends through an orientation slot of the outer tube to prohibit relative rotation of the latch tube and outer tube.

In some implementations, withdrawing the securing member from the coupling connection allows the actuation tube to move laterally relative to the latch tube to disengage the coupling connection.

In some implementations, an actuation assembly or an actuation mechanism for a device (e.g., a repair device, an implantable device, etc.) includes an actuation member releasably attached to an adjustable member that has a plurality of notches, a latch tube, and a release tube arranged between the adjustable member and the latch tube.

In some implementations, the latch tube has a plurality of latch members spaced apart longitudinally along the latch tube that are biased to move inward toward a latched condition.

In some implementations, the release tube prohibits the latch members from moving from an unlatched condition to the latched condition to permit the adjustable member can be moved to a desired position.

In some implementations, withdrawing the release tube distally permits at least one of the latch members to move inward from the unlatched condition to the latched condition by engaging one of the notches of the adjustable member.

In some implementations, the latch members are laser cut from the latch tube.

In some implementations, the actuation member is a suture.

In some implementations, a handle release assembly of a delivery system for a device has an actuation element extending from a distal end for engaging the device to a proximal end that is secured to an actuation element adaptor and a width adjustment element extending within the actuation element from a distal end for engaging the device to a proximal end that is secured to a width adjustment element adaptor.

In some implementations, the handle release assembly also comprises a connector body having a central lumen through which an actuation element and the width adjustment element extend.

In some implementations, the connector body has a coupling portion that includes one, some, or all of a retention groove, an inner body with attachment members for engaging the coupling portion and the retention groove of the connector body, a recess for receiving the actuation element adaptor, a catch, and side slots, an outer body having a proximal stop, a latch arm having a protrusion for engaging the catch of the inner body to prohibit relative movement of the outer body and the inner body, and side slots.

In some implementations, the width adjustment element adaptor extends through the side slots of the inner body and the side slots of the outer body, and a gripping portion slidably attached to the outer body. Sliding the gripping portion in the proximal direction exposes the latch arm of the outer body to permit the latch arm to disengage from the catch of the inner body.

In some implementations, the assembly is configured such that after disengaging the latch arm, the outer body is moved proximally by application of further proximal force on the gripping portion to move the outer body proximally so that the side slots engage the width adjustment element adaptor to move the width adjustment element in the proximal direction.

In some implementations, further proximal movement of the gripping portion and the outer body exposes the attachment members of the inner body and causes the width adjustment element adaptor to engage the side slots of the inner body to disengage the attachment members from the retention groove of the coupling portion of the connector body and to pull the actuation element in the proximal direction.

In some implementations, a handle release assembly of a delivery system for a device (e.g., a repair device, an implantable device, a delivery device, etc.) has an actuation element extending from a distal end for engaging the device to a proximal end that is secured to an actuation element adaptor and a width adjustment element extending within the actuation element from a distal end for engaging the device to a proximal end that is secured to a width adjustment element adaptor.

In some implementations, the handle release assembly also comprises a connector body having a central lumen through which an actuation element and the width adjustment element extend. In some implementations, the connector body including a coupling portion that includes one, some, or all of a retention groove, a moveable body comprising attachment members for engaging the coupling portion and the retention groove of the connector body, a recess for receiving the actuation element adaptor, and side slots.

In some implementations, the width adjustment element adaptor is configured to extend through the side slots of the moveable body, and a gripping portion slidably attached to the moveable body. In some implementations, the assembly can be configured such that sliding the gripping portion in the proximal direction engages the width adjustment element adaptor to move the width adjustment element in the proximal direction.

In some implementations, further proximal movement of the gripping portion and the outer body exposes the attachment members of the moveable body and causes the width adjustment element adaptor to engage the side slots of the inner body to disengage the attachment members from the retention groove of the coupling portion of the connector body and to pull the actuation element in the proximal direction.

In some implementations, a clasp for a device includes a fixed arm having a curved shape, a moveable arm having a curved shape that is complementary to the curved shape of the fixed arm, and a joint portion that hingeably connects the moveable arm to the fixed arm.

In some implementations, the clasp also includes a gripping protrusion or gripping member extending from at least one of the fixed arm and the moveable arm. In some implementations, the gripping protrusion or gripping member can have a rounded end. In some implementations, the gripping protrusion or gripping member can have a sharp end.

In some implementations, the curved shape is a sinusoidal shape, a triangular shape, or a sawtooth shape.

In some implementations, the curved shape has a peak arranged proximal to the joint portion and a trough arranged distal of the peak.

In some implementations, the curved shape has a trough arranged proximal to the joint portion and a peak arranged distal of the peak.

In some implementations, the curved shape has a plurality of peaks arranged between the joint portion and a distal end of the fixed arm and the moveable arm.

In some implementations, the curved shape has a plurality of troughs arranged between the joint portion and a distal end of the fixed arm and the moveable arm.

In some implementations, the clasp includes a gripping surface extending along at least a portion of one of the fixed arm and the moveable arm. The gripping surface can be formed by powder coating, laser printing, and/or an adhesive coating that releasably adheres to a valve leaflet.

In some implementations, the gripping surface comprises a plurality of teeth or a plurality of serrations.

In some implementations, a cover is included that covers at least one of the fixed arm and the moveable arm. The gripping surface can also have surface features that extend through the cover.

In some implementations, a device (e.g., a repair device, an implantable device, an implant, etc.) includes a first collar and a second collar, an expandable coaptation element extending between the first collar and the second collar, the expandable coaptation element having a middle portion between two end portions. In some implementations, the middle portion is more flexible than the two end portions so that moving the second collar in a proximal direction toward the first collar and causes the expandable coaptation element to contract.

In some implementations, the middle portion contracts more than the end portions when the second collar is retracted in the proximal direction. An optional biasing member can extend between the first collar and the second collar to bias the second collar toward or away from the first collar in a distal direction.

In some implementations, an actuation member can be attached to the second collar and moving the actuation member in the proximal direction can extend or compress the biasing member.

In some implementations, the expandable coaptation element can be made from a tube of braided wires that can be formed from a shape memory alloy, such as, for example nitinol, CuAlNi, NiTi, or another alloy comprising one or more of Zn, Cu, Au, and Fe. In some implementations, the braided wires in the middle portion of the expandable coaptation element can be thinner than the braided wires in the end portions. An optional cover can be used to cover the components of the expandable coaptation element.

In some implementations, a device (e.g., a repair device, an implantable device, etc.) includes a first nut and a second nut, wherein at least one of the first nut and the second nut comprises a threaded opening. In some implementations, a pair of flexible struts extends between the first nut and the second nut, each of the pair of flexible struts comprising a first threaded end, a second threaded end, and a flexible middle portion.

In some implementations, the first threaded end of each flexible strut engages the threaded opening of the first nut and the second threaded end of each flexible struts engages the threaded opening of the second nut.

In some implementation, the device is configured such that rotation of the first nut relative to the first threaded ends of the pair of flexible struts moves the first threaded ends towards and away from the first nut and rotation of the second nut relative to the second threaded ends of the pair of flexible struts moves the second threaded ends towards and away from the second nut.

In some implementation, the device is configured such that moving at least one of the first threaded ends away from the first nut and the second threaded ends away from the second nut causes the flexible middle portion of each flexible struts to expand laterally outward.

In some implementations, one or both of the first nut and the second nut can include an engagement portion for engaging the flexible middle portion to move the flexible middle portion laterally inward. The threaded openings of the first nut and the second nut can have opposite-handed threads. One or both of the first nut and the second nut can be rotated to expand and contract the flexible middle portion of the flexible struts. An optional cover can be used to cover the components of the expandable coaptation element.

In some implementations, a device (e.g., a repair device, an implantable device, etc.) includes a first actuation member that is moveably attached to a second actuation member. In some implementations, the device can be configured such that engaging at least one of the first actuation member and the second actuation member moves the first actuation member and the second actuation member toward each other.

In some implementations, the device includes an expandable coaptation element that comprises an expansion member and a spreader member that are both arranged between the first and the second actuation members. In some implementations, the device can be configured such that movement of the first and the second actuation member towards each other causes the spreader member to engage and expand the expansion member.

In some implementations, the first actuation member can include a distally extending threaded rod; wherein the distally extending threaded rod extends through the expandable coaptation element; and wherein the second actuation member comprise a threaded opening for engaging the distally extending threaded rod of the first actuation member.

In some implementations, the expansion member can have two expandable rings arranged between the spreader member and each of the first second member and the second actuation member. In some implementations, the spreader member comprises a tapered end for engaging the expansion member that can include a corresponding chamfered inner diameter.

In some implementations, the actuation assembly or mechanism can include a threaded rod with first and second threaded portions with opposite-handed threads that engage the first and second actuation members, respectively. Rotating the threaded rod moves the actuation members towards and away from each other to cause the expandable coaptation element to expand and retract. An optional cover can be used to cover the components of the expandable coaptation element.

In some implementations, a device includes a fixed actuation member, a moveable actuation member, a threaded shaft extending through the moveable actuation member and rotatably attached to the first actuation member, and a plurality of struts extending between the fixed actuation member and the moveable actuation member.

In some implementations, each strut is made from a plurality of rigid portions connected together by a plurality of hinge portions.

In some implementations, the device is configured such that rotating the threaded shaft causes the moveable actuation member to move towards and away from the fixed actuation member to cause the plurality of struts to expand and contract, respectively. In some implementations, each of the struts can be made from three rigid portions, and the second—i.e., the middle—portion can include a mounting location for attaching other components of the device. Any number of struts can be used, for example, two struts or four struts.

In some implementations, when the device has four struts, the four struts can be arranged in two pairs of opposing struts. In some implementations, each strut of one of the two opposing pairs of struts can include a mounting location. The different opposing pairs of struts can expand at different rates.

In some implementations, a device (e.g., a repair device, an implantable device, etc.) includes a first actuation member, a second actuation member, and a threaded shaft having a first threaded portion for engaging the first actuation member and a second threaded portion for engaging the second actuation member. The first and second threaded portion include opposite-handed threads.

In some implementations, a plurality of struts extends between the first actuation member and the second actuation member, each strut being made from a plurality of rigid portions connected together by a plurality of hinge portions.

In some implementations, the device is configured such that rotating the threaded shaft causes the first actuation member and the second actuation member to move towards and away from each other to cause the plurality of struts to expand and contract, respectively. In some implementations, each of the struts can be made from three rigid portions, and the second—i.e., the middle—portion can include a mounting location for attaching other components of the device. Any number of struts can be used, for example, two struts or four struts.

In some implementations, when the device has four struts, the four struts can be arranged in two pairs of opposing struts. Each strut of one of the two opposing pairs of struts can include a mounting location. The different opposing pairs of struts can expand at different rates.

In some implementations, a device (e.g., a repair device, an implantable device, a delivery device, etc.) has an outer tube attached to a fixed actuation member and the outer tube includes a plurality of openings. In some implementations, a latch tube is attached to a moveable actuation member, the latch tube having a latch member.

In some implementations, a securing member extends through the latch tube and has a tapered end for engaging the latch member.

In some implementations, a plurality of struts extend between the fixed actuation member and the moveable actuation member, with each strut including a plurality of rigid portions connected together by a plurality of hinge portions.

In some implementations, when the securing member is inserted into the latch tube, the tapered distal end engages the latch member and retains the latch member in an unlatched condition so that the latch tube can be moved to a desired position in the outer tube.

In some implementations, the securing member can be withdrawn from the latch tube to disengage the securing member from the latch member to facilitate movement of the latch member to the latched condition wherein the latch member engages one of the plurality of openings of the outer tube.

In some implementations, the device is configured such that extending and retracting the latch tube causes the moveable actuation member to move towards and away from the fixed actuation member to cause the plurality of struts to expand and contract, respectively.

The latch member can be laser cut from the latch tube and both can be formed from a shape memory alloy with the latch member being shape set in the latched condition. In some implementations, each strut has two rigid portions that are joined by a hinge portion. The struts can be joined into pairs at proximal and distal ends and can also be cut together from a sheet of material. Two pairs of struts can be connected to each other by a side plate that can optically include a mounting portion.

In some implementations, a device has an actuation spool, a central frame extending from the actuation spool, a round shaped pivoting frame pivotably attached to the central frame, and an actuation member that extends from the actuation spool to the pivoting frame. In some implementations, the device is configured such that rotation of the actuation spool retracts and releases the actuation member to cause the pivoting frame to pivot.

In some implementations, the device can have a plurality of pivoting frames and one or more of the plurality of pivoting frames can be smaller than the rest of the pivoting frames so that the combination of the pivoting frames provides a smooth three-dimensional shape. In some implementations, the shape can be configured to fill a similarly shaped gap between the native leaflets.

In some implementations, when the device includes a plurality of pivoting frames, at least one actuation element can attach adjacent pivoting frames so that pivoting a first pivoting frame causes a second pivoting frame to pivot.

In some implementations, when the actuation spool is rotated a first half of the pivoting frame moves toward the actuation spool and a second half of the pivoting frame moves away from the actuation spool. In other implementations, when the actuation spool is rotated a first half of the pivoting frame moves toward the actuation spool and a second half of the pivoting frame also moves toward from the actuation spool. The pivoting frame can extend through the central frame or can end at the central frame.

In some implementations, the actuation member is released when the actuation spool is rotated in a first direction and the actuation member is retracted when the spool is rotated in a second direction.

In some implementations, a first actuation member is released, and a second actuation member is retracted when the actuation spool is rotated in a first direction and the first actuation member is retracted and the second actuation member is released when the actuation spool is rotated in a second direction.

In some implementations, a first actuation member is released, and a second actuation member is released when the actuation spool is rotated in a first direction and the first actuation member is retracted and the second actuation member is retracted when the actuation spool is rotated in a second direction.

In some implementations, a heart valve repair system includes a device (e.g., a repair device, an implantable device, etc.) that has a proximal collar and a delivery system that includes a distal coupler and an actuation member. In some implementations, the proximal collar has a pair of protrusions that extend radially inward to engage openings of a pair of moveable arms of the distal coupler.

In some implementations, the moveable arms can be moved between an engaged position and a disengaged position to engage and disengaged from the protrusions.

In some implementations, the actuation member extends through the distal coupler and between the pair of movable arms to retain the moveable arms in the engaged position.

In some implementations, the system is configured such that retracting the actuation member through the distal coupler allows the pair of moveable arms to disengage from the pair of protrusions.

In some implementations, the moveable arms can be biased in the inward or disengaging direction and can also include engagement portions for engaging the actuation member that can have a rounded shape. In some implementations, the engagement portions are compressed between the actuation member and the moveable arms so that the moveable arms are pressed against the pair of protrusions of the proximal collar.

In some implementations, a device (e.g., a repair device, an implantable device, etc.) includes an outer tube, an inner tube, and one or more expandable coaptation members. In some implementations, the outer tube comprises an opening. In some implementations, the inner tube is arranged concentrically within the outer tube.

In some implementations, the one or more expandable coaptation members extend from a first end to a second end.

In some implementations, the first end is hingeably attached to the outer tube and the second end is hingeably attached to the inner tube through the opening in the outer tube.

In some implementations, the device can be configured such that rotating the outer tube relative to the inner tube causes the expandable coaptation member to expand radially outward.

In some implementations, one or more expandable coaptation members comprises a plurality of links connected together by a plurality of hinge portions.

In some implementations, the one or more expandable coaptation members are cut from the outer tube.

In some implementations, the device comprises three expandable coaptation members that are radially spaced apart from each other equally.

In some implementations, the hinge portions comprise a plurality of cuts.

In some implementations, the one or more expandable coaptation members vary in width between the first end and the second end.

In some implementations, the one or more expandable coaptation members are formed from a shape memory alloy.

In some implementations, the one or more expandable coaptation members are biased in the expansion direction.

In some implementations, a heart valve repair system includes a device (e.g., a repair device, an implantable device, etc.) and a delivery system. In some implementations, the device comprises a coaptation portion and an anchor portion.

In some implementations, the coaptation portion comprising an outer tube comprising an opening, an inner tube arranged concentrically within the outer tube, one or more expandable coaptation members extending from a first end to a second end.

In some implementations, the first end is hingeably attached to the outer tube and the second end is hingeably attached to the inner tube through the opening in the outer tube.

In some implementations, the delivery system comprises a delivery sheath and an actuation member.

In some implementations, the actuation member engages one of the inner tube and the outer tube such that rotating the actuation member causes the one or more expandable coaptation members to expand radially outward.

In some implementations, the expandable coaptation member are biased in an expansion direction.

In some implementations, the device further comprises a cover that covers the one or more expandable coaptation member.

In some implementations a device includes a flexible enclosure and an elongated filling element. In some implementations, the flexible enclosure forms an expandable cavity. In some implementations, the flexible enclosure is expandable from an unexpanded to an expanded condition.

In some implementations, the elongated filling element is configured to fill at least a portion of the expandable cavity when the flexible enclosure is in the expanded condition.

In some implementations, the elongated filling element provides resistance to compression of the flexible enclosure in the expanded condition.

In some implementations, the flexible enclosure is formed from a woven textile material. In some implementations, the flexible enclosure is formed from a molded polymer material.

In some implementations, the elongated filling element comprises a metal coil. In some implementations, the elongated filling element comprises a polymer coil. In some implementations, the elongated filling element comprises a radiopaque material.

In some implementations, the radiopaque material is formed into a plurality of markers attached to the elongated filling element.

In some implementations, a second elongated filling element disposed within the expandable cavity.

In some implementations, the second elongated filling element is formed from a material that is different from the elongated filling element.

In some implementations, the device further comprises a plurality of pieces of filling material.

In some implementations, at least one of the elongated filling element, the second elongated filling element, and the plurality of pieces of filling material are coated with a hydrogel coating.

In some implementations, the flexible enclosure comprises an expandable frame formed from a shape memory alloy.

In some implementations, a heart valve repair system includes a device (e.g., a repair device, an implantable device, etc.) and a delivery system. In some implementations, the device comprises a coaptation portion and an anchor portion.

In some implementations, the coaptation portion comprises a flexible enclosure that is expandable from an unexpanded to expanded condition and that forms an expandable cavity. In some implementations, the coaptation portion includes an elongated filling element configured to fill at least a portion of the expandable cavity when the flexible enclosure is in the expanded condition.

In some implementations, the elongated filling element provides resistance to compression of the flexible enclosure in the expanded condition.

In some implementations, the delivery system comprises a delivery sheath and an actuation member.

In some implementations, the actuation member facilitates the expansion of the flexible enclosure.

In some implementations, the elongated filling element is configured to be delivered into the expandable cavity via the delivery sheath.

In some implementations, the actuation member comprises a tube for filling the expandable cavity with saline to cause the flexible enclosure to expand.

In some implementations, the flexible enclosure is caused to expand by delivery of the elongated filling element.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of a device or implant, in various stages of deployment;

FIG. 15 shows an example of a device or implant that is similar to the device illustrated by FIGS. 8-14, but where the paddles are independently controllable;

FIGS. 16-21 show the example device or implant of FIGS. 8-14 being delivered and implanted within a native valve;

FIG. 22 shows a perspective view of an example device or implant in a closed position;

FIG. 23 shows a front view of the device or implant of FIG. 22;

FIG. 24 shows a side view of the device or implant of FIG. 22;

FIG. 25 shows a front view of the device or implant of FIG. 22 with a cover covering the paddles and a coaptation element or spacer;

FIG. 26 shows a top perspective view of the device or implant of FIG. 22 in an open position;

FIG. 27 shows a bottom perspective view of the device or implant of FIG. 22 in an open position;

FIG. 28A shows a clasp for use in a device or implant;

FIG. 28B shows a perspective view of an example clasp of an example device or implant in a closed position;

FIG. 29 shows a portion of native valve tissue grasped by a clasp;

FIG. 30 shows a side view of an example device or implant in a partially open position with clasps in a closed position;

FIG. 31 shows a side view of an example device or implant in a partially open position with clasps in an open position;

FIG. 32 shows a side view of an example device or implant in a half-open position with clasps in a closed position;

FIG. 33 shows a side view of an example device or implant in a half-open position with clasps in an open position;

FIG. 34 shows a side view of an example device or implant in a three-quarters-open position with clasps in a closed position;

FIG. 35 shows a side view of an example device or implant in a three-quarters-open position with clasps in an open position;

FIG. 36 shows a side view of an example device in a fully open or full bailout position with clasps in a closed position;

FIG. 37 shows a side view of an example device in a fully open or full bailout position with clasps in an open position;

FIGS. 38-49 show the example device or implant of FIGS. 30-38, including a cover, being delivered and implanted within a native valve;

FIG. 50 is a schematic view illustrating a path of native valve leaflets along each side of a coaptation element or spacer of an example valve repair device or implant;

FIG. 51 is a top schematic view illustrating a path of native valve leaflets around a coaptation element or spacer of an example valve repair device or implant;

FIG. 52 illustrates a coaptation element or spacer in a gap of a native valve as viewed from an atrial side of the native valve;

FIG. 53 illustrates a valve repair device or implant attached to native valve leaflets with the coaptation element or spacer in the gap of the native valve as viewed from a ventricular side of the native valve;

FIG. 54 is a perspective view of a valve repair device or implant attached to native valve leaflets with the coaptation element or spacer in the gap of the native valve shown from a ventricular side of the native valve;

FIG. 55 shows a perspective view of an example device or implant in a closed position;

FIG. 56A illustrates a valve repair device with paddles in an open position;

FIG. 56B illustrates the valve repair device of FIG. 56A, in which the paddles are in the open position and gripping members are moved to create a wider gap between the gripping members and paddles;

FIG. 56C illustrates the valve repair device of FIG. 56A, in which the valve repair device is in the position shown in FIG. 56A with valve tissue placed between the gripping members and the paddles;

FIG. 56D illustrates the valve repair device of FIG. 56A, in which the gripping members are moved to lessen the gap between the gripping members and the paddles;

FIGS. 56E-56F illustrate the movement of the paddles of the valve repair device of FIG. 56A from the open position to a closed position;

FIG. 56G illustrates the valve repair device of FIG. 56A in a closed position, in which the gripping members are engaging valve tissue;

FIG. 56H illustrates the valve repair device of FIG. 56A after being disconnected from a delivery device and attached to valve tissue, in which the valve repair device is in a closed and locked condition;

FIG. 57 shows a top view of an example device or implant having anchors that each include a plurality of paddles and a plurality of clasps such that each clasp corresponds to an associated paddle;

FIG. 58 shows a front view of the example device or implant of FIG. 57;

FIG. 59 shows a side view of the example device or implant of FIG. 57;

FIG. 60 shows a top view of an example device or implant that is similar to the example device of FIG. 57 except only a portion of the paddles of each anchor include a corresponding clasp;

FIG. 61 shows a front view of the example device or implant of FIG. 60;

FIG. 62 shows a side view of the example device or implant of FIG. 60;

FIG. 63 shows a top view of an example device or implant that is similar to the example device of FIG. 60 except an inner paddle of each anchor has a longer length than outer paddles of the anchor;

FIG. 64 shows a front view of the example device or implant of FIG. 63;

FIG. 65 shows a side view of the example device or implant of FIG. 63;

FIG. 66 shows a top view of an example device or implant that is similar to the example device of FIG. 60 except an inner paddle of each anchor has a shorter length than outer paddles of the anchor;

FIG. 67 shows a front view of the example device or implant of FIG. 66;

FIG. 68 shows a side view of the example device or implant of FIG. 66;

FIGS. 69-73 show the example device or implant of FIG. 57 during various stages of deployment;

FIG. 74 shows a top view of an example device or implant having anchors that each include a plurality of paddle members and a plurality of clasps such that each clasp corresponds to an associated paddle member;

FIG. 75 shows a front view of the example device or implant of FIG. 74;

FIG. 76 shows a side view of the example device or implant of FIG. 74;

FIG. 77 shows a top view of an example device or implant that is similar to the example device of FIG. 74 except only a portion of the paddle members of each anchor include a corresponding clasp;

FIG. 78 shows a front view of the example device or implant of FIG. 77;

FIG. 79 shows a side view of the example device or implant of FIG. 77;

FIG. 80 shows a top view of an example device or implant that is similar to the example device of FIG. 77 except an inner paddle member of each anchor has a longer length than outer paddle members of the anchor;

FIG. 81 shows a front view of the example device or implant of FIG. 80;

FIG. 82 shows a side view of the example device or implant of FIG. 80;

FIG. 83 shows a top view of an example device or implant that is similar to the example device of FIG. 77 except an inner paddle member of each anchor has a shorter length than outer paddle members of the anchor;

FIG. 84 shows a front view of the example device or implant of FIG. 83;

FIG. 85 shows a side view of the example device or implant of FIG. 83;

FIGS. 86A, 87A, and 88-90 show the example device or implant of FIG. 57 during various stages of deployment;

FIGS. 86B and 87B illustrate an example similar to the example illustrated by FIGS. 86A and 87A where the paddle portions are in an extended position;

FIG. 91 shows a perspective view of an example paddle frame for a device or implant;

FIG. 92 shows a partial view of the paddle frame of FIG. 91 when the paddle frame is in a narrowed position;

FIG. 93 shows the paddle frame of FIG. 91 disposed within a delivery system;

FIG. 94 shows an example device or implant that includes the paddle frame of FIG. 91 when the device or implant is in an open position;

FIG. 95 shows the paddle frame of FIG. 91 when the paddle frame is in the narrowed position;

FIG. 96 shows a perspective view of an example paddle frame for a device or implant;

FIG. 97 shows a partial view of the paddle frame of FIG. 96;

FIG. 98 shows a partial front view of an example device that includes an example paddle frame where the device or implant is in a closed position;

FIG. 99 shows a partial front view of an example device that includes an example paddle frame where the device or implant is in a closed position;

FIG. 100 shows a partial front view of an example device that includes an example paddle frame where the device or implant is in a closed position;

FIG. 101 shows a partial front view of the device or implant of FIG. 98 where the device or implant is in an open position;

FIG. 102 shows a partial front view of the device or implant of FIG. 99 where the device or implant is in an open position;

FIG. 103 shows a partial front view of the device or implant of FIG. 100 where the device or implant is in an open position;

FIG. 104 shows a partial side view of the device or implant of FIG. 98 where the device or implant is in an open position;

FIG. 105 shows a partial side view of the device or implant of FIG. 99 where the device or implant is in an open position;

FIG. 106 shows a partial side view of the device or implant of FIG. 100 where the device or implant is in an open position;

FIG. 107 shows a front view of an example paddle frame for a device or implant;

FIG. 108 shows a front view of the example paddle frame of FIG. 107 when the paddle frame is in a narrowed position;

FIG. 109 shows a front view of an example paddle frame for a device or implant;

FIG. 110 shows a front view of the example paddle frame of FIG. 109 when the paddle frame is in a narrowed position;

FIG. 111 shows a front view of an example paddle frame for a device or implant;

FIG. 112 shows a front view of an example paddle frame for a device or implant;

FIG. 113 shows a front view of an example configuration of the example paddle frame of FIG. 112;

FIG. 114 shows a front view of an example configuration of the example paddle frame of FIG. 112;

FIG. 115 shows a front view of an example paddle frame for a device or implant;

FIG. 116 shows a top view of the example paddle frame of FIG. 115;

FIG. 117 shows a perspective view of an example device or implant that includes an example paddle frame where the device or implant is in an open position;

FIG. 118 shows a bottom view of the device or implant of FIG. 117;

FIG. 119 shows a front view of the device or implant of FIG. 117 where the device or implant is in a closed position;

FIG. 120 shows a side view of the device or implant of FIG. 117 attached to a native valve of a heart;

FIG. 121 shows a bottom view of the device or implant of FIG. 117 attached to a native valve of a heart;

FIG. 122 shows a front view of an example device or implant where the device or implant is in a closed position;

FIG. 123 shows the example device or implant of FIG. 122 where the device or implant is in an open position;

FIG. 124 shows an example paddle frame of the device or implant of FIG. 122 when the device or implant is in the open position;

FIG. 125 shows a front view of an example paddle frame for a device or implant where the paddle frame is in a narrowed position;

FIG. 126 shows the example paddle frame of FIG. 125 where the paddle frame is in an expanded position;

FIG. 127 shows a perspective view of an example device or implant that includes an example paddle frame where the device includes an example means of moving the paddle frame from a normal position to a narrowed position;

FIG. 128 shows the paddle frame of FIG. 127 in the narrowed position;

FIG. 129 shows a perspective view of the device or implant of FIG. 127 except that the device includes an example means of moving the paddle from the normal position to the narrowed position;

FIG. 130 shows a perspective view of the device or implant of FIG. 127 except that the device includes an example means of moving the paddle from the normal position to the narrowed position;

FIG. 131 shows a perspective view of an example device or implant that includes an example paddle frame;

FIG. 132 shows the device or implant of FIG. 131 with an example means for moving the paddle frame from a normal position to a narrowed position;

FIG. 133 shows the device or implant of FIG. 131 with an example means for moving the paddle frame from a normal position to a narrowed position;

FIG. 134 shows the device or implant of FIG. 131 with an example means for moving the paddle frame from a normal position to a narrowed position;

FIG. 135 shows the device or implant of FIG. 131 with an example means for moving the paddle frame from a normal position to a narrowed position;

FIG. 136 shows the device or implant of FIG. 131 with an example means for moving the paddle frame from a normal position to a narrowed position;

FIG. 137 shows a front view of an example paddle frame for a device or implant;

FIG. 138 shows a pair of the example paddle frames of FIG. 137 positioned adjacent to each other;

FIG. 139 shows a side view of an example of a device or implant that includes the paddle frame of FIG. 137, where the paddle frame is in a narrowed position;

FIG. 140 shows a side view of the device or implant of FIG. 139 where the paddle frame is in an expanded position;

FIG. 141 shows a partial side view of the device or implant of FIG. 139 where the paddle frame is in the narrowed position;

FIG. 142 shows a partial side view of the device or implant of FIG. 139 where the paddle frame is in the expanded position;

FIG. 143 shows a perspective view of the device or implant of FIG. 139 with the paddle frame of FIG. 137;

FIG. 144 shows a front view of the device or implant of FIG. 139 with the paddle frame of FIG. 137;

FIG. 145 show a perspective view of an example of inner and outer paddles for the device or implant of FIG. 139;

FIG. 146 shows a side view of the inner and outer paddles of FIG. 145;

FIG. 147 shows a top view of the inner and outer paddles of FIG. 145;

FIG. 148 shows a perspective view of an example connection between the paddles of FIG. 146 and the paddle frame of FIG. 137;

FIG. 149 shows a front view of an example paddle frame for a device or implant;

FIG. 150 shows a front view of an example paddle frame for a device or implant;

FIG. 151 shows a front view of an example paddle frame for a device or implant;

FIG. 152 shows a front view of an example paddle frame for a device or implant;

FIGS. 153-155 show front view of various configurations for an example paddle frame for a device or implant;

FIG. 156 shows a front view of an example paddle frame for a device or implant;

FIG. 157 shows a front view of an example paddle frame for a device or implant, where the paddle frame is shown in an expanded position;

FIG. 158 shows a front view of the example paddle frame of FIG. 157, where the paddle frame is shown in a narrowed position;

FIG. 159 shows a front view of an example paddle frame for a device or implant;

FIG. 160 shows a left side view of the paddle frame of FIG. 159;

FIG. 161 shows a top view of the paddle frame of FIG. 159;

FIG. 162 shows a perspective view of an example of a device or implant that includes the paddle frame of FIG. 159;

FIG. 163 shows a front view of the device or implant of FIG. 162 that includes the paddle frame of FIG. 159;

FIGS. 164-168 shows the device or implant of FIG. 162 having an example means for moving the paddle frame of FIG. 159 between an expanded position and narrowed positions;

FIG. 169 shows a perspective view of an example of a paddle and coaptation element frame assembly for a device or implant;

FIG. 170 shows a rear view of the paddle and coaptation element frame assembly of FIG. 169;

FIG. 171 shows a perspective view of an example of a device or implant that includes the paddle and coaptation element frame assembly of FIG. 171, where the coaptation element frame is in a narrowed position;

FIG. 172 shows a perspective view of the device or implant of FIG. 171 where the coaptation element frame is in an expanded position;

FIG. 173 shows a top view of the device or implant of FIG. 171 with the coaptation element frame in the narrowed position;

FIG. 174 shows a top view of the device or implant of FIG. 172 with the coaptation element frame in the expanded position;

FIG. 175 shows a rear view of the paddle and coaptation element frame assembly of FIG. 169 when in the narrowed position, where the paddle and coaptation element frame assembly is attached to inner and outer paddles of the anchor portion of a device or implant;

FIG. 176 shows a rear view of the paddle and coaptation element frame assembly of FIG. 169 when in the expanded position, where the paddle and coaptation element frame assembly is attached to inner and outer paddles of the anchor portion of a device or implant;

FIG. 177 shows a perspective view of the paddle and coaptation element frame assembly of FIG. 169 when in the narrowed position, where the paddle and coaptation element frame assembly is attached to inner and outer paddles of the anchor portion of the device or implant;

FIG. 178 shows a top view of the paddle and coaptation element frame assembly of FIG. 169 when in the expanded position, where the paddle and coaptation element frame assembly is attached to inner and outer paddles of the anchor portion of a device or implant;

FIG. 179 shows a perspective view of the paddle and coaptation element frame assembly of FIG. 169 when in the expanded position;

FIG. 180 shows a perspective view of the coaptation element frame of the paddle and coaptation element frame assembly of FIG. 169, where the coaptation element frame is attached to an inner paddle of the anchor portion of a device or implant;

FIG. 181 shows a front view of the frame of the paddle and coaptation element assembly of FIG. 169 when in the narrowed position;

FIG. 182 shows a side view of the coaptation element frame of FIG. 181;

FIG. 183 shows a top view of the coaptation element frame of FIG. 181;

FIG. 184 shows a perspective view of the coaptation element frame of FIG. 181;

FIG. 185 shows a front view of the coaptation element frame of FIG. 181 when in the expanded position;

FIG. 186 shows a side view of the coaptation element frame of FIG. 185;

FIG. 187 shows a top view of the coaptation element frame of FIG. 185;

FIG. 188 shows a perspective view of the coaptation element frame of FIG. 185;

FIG. 189 shows a perspective view of a pair of example paddle frames for a pair of anchors of a device or implant;

FIG. 190 shows a front view of the paddle frames of FIG. 189;

FIG. 191 shows a top view of the paddle frames of FIG. 189;

FIG. 192 shows a side view of the paddle frames of FIG. 189;

FIG. 193 shows a top view of an example of a device or implant that includes one of the paddle frames of FIG. 189, where the paddle frame is in an expanded position;

FIG. 194 shows a top view of the device or implant of FIG. 193 where the paddle frame is in the narrowed position;

FIG. 195 shows a ventricular side view of the native valve with the device or implant of FIG. 193 being positioned to connect to the native valve;

FIG. 196 shows an atrial side view of an example device or implant attached to a native valve of the heart;

FIG. 197 shows an atrial side view of the device or implant of FIG. 196 attached to the native valve where tissue ingrowth has covered the device;

FIG. 198 shows a front view of the device or implant of FIG. 196 attached to the native valve where tissue ingrowth has covered the device;

FIG. 199 shows an atrial side view of an example device or implant attached to a native valve of the heart where the device includes an example coaptation extension member;

FIG. 200 shows an atrial side view of the device or implant of FIG. 199 attached to the native valve where tissue ingrowth has covered the device;

FIG. 201 shows an atrial side view of an example device or implant attached to a native valve of the heart where the device includes an example coaptation extension member;

FIG. 202 shows an atrial side view of the device or implant of FIG. 201 attached to the native valve where tissue ingrowth has covered the device;

FIG. 203 shows a front view of an example device or implant attached to a native valve of the heart where the device includes an example coaptation extension member;

FIG. 204 shows a front view of the device or implant of FIG. 203 attached to the native valve where tissue ingrowth has covered the device;

FIG. 205 shows a front view of an example device or implant attached to a native valve of the heart where the device includes an example coaptation extension member;

FIG. 206 shows a front view of the device or implant of FIG. 205 attached to the native valve where tissue ingrowth has covered the device;

FIG. 207 shows an atrial side view of an example device or implant attached to a native valve of the heart where the device includes an example coaptation extension member;

FIG. 208 shows a front view of an example device or implant attached to a native valve of the heart where the device includes an example coaptation extension member;

FIG. 209 shows a front view of an example device or implant attached to a native valve of the heart where the device includes an example coaptation extension member;

FIGS. 210-214 illustrate an example of a coupling between an actuation element and a component of a device or implant;

FIGS. 215-218 illustrate an example of a coupling between an actuation element and a component of a device or implant;

FIGS. 219-222 illustrate an example of a coupling between an actuation element and a component of a device or implant;

FIGS. 223-224 illustrate an example of a coupling between an actuation element and a component of a device or implant;

FIGS. 225-227 illustrate an example of a coupling between an actuation element and a component of a device or implant;

FIGS. 228-230 illustrate an example of a coupling between an actuation element and a component of a device or implant;

FIGS. 231-232 illustrate an example of a coupling between an actuation element and a component of a device or implant;

FIG. 233 illustrates an example of a coupling between an actuation element and a component of a device or implant;

FIG. 234 illustrates an example of a coupling between an actuation element and a component of a device or implant;

FIG. 235 illustrates an example of a coupling between an actuation element and a component of a device or implant;

FIG. 236 illustrates an example of a coupling between an actuation element and a component of a device or implant;

FIG. 237 illustrates an example of a coupling between an actuation element and a component of a device or implant;

FIG. 238 illustrates an example of a coupling between an actuation element and a component of a device or implant;

FIG. 239 illustrates an example of a coupling between an actuation element and a component of a device or implant;

FIG. 240 illustrates an example of a coupling between an actuation element and a component of a device or implant;

FIG. 241 illustrates an example of a coupling between an actuation element and a component of a device or implant;

FIG. 242 illustrates an example of a coupling between an actuation element and a component of a device or implant;

FIG. 243 illustrates an example of a coupling between an actuation element and a component of a device or implant;

FIG. 244 illustrates an example of a coupling between an actuation element and a component of a device or implant;

FIGS. 245-250 illustrate an example of a coupling between an actuation element and a component of a device or implant;

FIGS. 251-252 illustrate an example of a coupling between an actuation element and a component of a device or implant;

FIG. 253 illustrates an example of a coupling between an actuation element and a component of a device or implant;

FIGS. 254-255 illustrate an example of a coupling between an actuation element and a component of a device or implant;

FIG. 256 illustrates an example of a coupling between an actuation element and a component of a device or implant;

FIG. 257 illustrates an example of a coupling between an actuation element and a component of a device or implant;

FIGS. 258-259 illustrate an example of a coupling between an actuation element and a component of a device or implant;

FIGS. 260-261 illustrate an example of a coupling between an actuation element and a component of a device or implant;

FIG. 262 illustrates an example of a coupling between an actuation element and a component of a device or implant;

FIGS. 263-264 illustrate an example of a coupling between an actuation element and a component of a device or implant;

FIGS. 265-266 illustrate an example of a coupling between an actuation element and a component of a device or implant;

FIGS. 267-268 illustrate an example of a coupling between an actuation element and a component of a device or implant;

FIGS. 269-270 illustrate an example of a coupling between an actuation element and a component of a device or implant;

FIG. 271 illustrates an example of a coupling between an actuation element and a component of a device or implant;

FIG. 272 illustrates an example of a coupling between an actuation element and a component of a device or implant;

FIG. 273 illustrates an example of a coupling between an actuation element and a component of a device or implant;

FIG. 274 illustrates an example of a coupling between an actuation element and a component of a device or implant;

FIG. 275 illustrates an example of a coupling between an actuation element and a component of a device or implant;

FIG. 276 illustrates an example of a width adjustment device or control device;

FIG. 277 illustrates an example of a width adjustment device or control device;

FIG. 278 illustrates an example of a pulley arrangement.

FIG. 279 is a top view of the width adjustment device or control device illustrated by FIG. 277;

FIG. 280 is a bottom view of the width adjustment device or control device illustrated by FIG. 277;

FIGS. 281 and 282 illustrate an example of a width adjustment device or control device;

FIGS. 283-285 illustrate an example of a width adjustment device or control device;

FIG. 286 illustrates an example of a paddle frame;

FIG. 287 illustrates an example of a width adjustment device or control device coupled to a paddle frame;

FIG. 288 illustrates an example of a width adjustment device or control device coupled to a paddle frame;

FIG. 289 illustrates an example of an adjustable paddle frame assembly;

FIG. 290 illustrates an example of an adjustment mechanism for the adjustable paddle assembly of FIG. 289;

FIG. 291 illustrates an example of an adjustable paddle frame assembly;

FIG. 292 illustrates an example of a width adjustment device or control device;

FIG. 293 illustrates an example of an adjustable paddle frame assembly;

FIG. 294 illustrates an example of an adjustable paddle frame assembly;

FIG. 295 illustrates an example of an adjustable paddle frame assembly;

FIG. 296 illustrates an example of an adjustment member of the adjustable paddle frame assemblies of FIGS. 294 and 295;

FIG. 297 illustrates an example of an adjustable paddle frame assembly;

FIGS. 298-300 illustrate an example of a width adjustment device or control device;

FIG. 301 shows a front cross-section view of a device or implant;

FIG. 302 shows a perspective cross section view of the device/implant of FIG. 301;

FIG. 303 shows a perspective view of the device/implant of FIG. 301;

FIG. 304 shows a side view of the device/implant of FIG. 301;

FIG. 305 shows a top view of the device/implant of FIG. 301;

FIGS. 306-311 show a partial view of the device/implant of FIG. 301 in various stages of assembly;

FIG. 312 shows a front view of the device/implant of 301 in an expanded position;

FIG. 313 shows a side view of the device/implant of 301 in an expanded position;

FIG. 314 shows a top view of the device/implant of 301 in an expanded position;

FIG. 315 shows a front view of the device/implant of 301 in a narrowed position;

FIG. 316 shows a side view of the device/implant of 301 in a narrowed position;

FIG. 317 shows a top view of the device/implant of 301 in a narrowed position;

FIG. 318 shows a front cross-section view of an example of a device or implant;

FIG. 319 shows a side view of the device/implant of FIG. 318;

FIGS. 320-323 shows front views of the device/implant of FIG. 318 at various positions moving from an expanded position to a narrowed position;

FIG. 324 shows a front view of an example of a portion of a paddle frame for a device or implant;

FIG. 325 shows a perspective view of the frame of FIG. 324;

FIG. 326 shows a top view of the frame of FIG. 324;

FIG. 327 shows a side view of the frame of FIG. 324;

FIGS. 328-331 shows front views of the device/implant of FIG. 324 at various positions moving from an expanded position to a narrowed position;

FIG. 332 shows a perspective view of an example of a portion of a paddle frame for a device or implant;

FIG. 333 shows a front view of the frame of FIG. 332 attached to an anchor;

FIG. 334 shows a partial cross section front view of the frame of FIG. 332 as part of the device or implant;

FIG. 335 shows the frame of FIG. 332 attached to a width adjustment device of a device or implant;

FIG. 336 shows a perspective view of a device or implant using the frame of FIG. 332;

FIG. 337 shows a front view of the device of FIG. 332 in an expanded position;

FIG. 338 shows a front view of the device of FIG. 332 in a narrowed position;

FIG. 339 shows a side view of the device of FIG. 332 in an expanded position;

FIG. 340 shows a side view of the device of FIG. 332 in a narrowed position;

FIG. 341 shows a top view of the device of FIG. 332 in an expanded position;

FIG. 342 shows a top view of the device of FIG. 332 in a narrowed position;

FIG. 343 shows a front view of a device or implant depicting two examples of paddle frames for the device;

FIG. 344 shows a front view of an example paddle frame for a device or implant;

FIGS. 345-347 show front views of the frame of FIG. 332 in various positions from an expanded position and a narrowed position;

FIG. 348 shows a front view of an example paddle frame for a device or implant;

FIG. 349 shows a perspective view of the frame of FIG. 348 as part of a device or implant;

FIG. 350 shows a front view of an example paddle frame for a device or implant;

FIG. 351 shows a perspective view of the frame of FIG. 350;

FIG. 352 shows a top view of the frame of FIG. 350;

FIG. 353 shows a side view of the frame of FIG. 350;

FIG. 354 shows a front view of an example paddle frame for a device or implant;

FIG. 355 shows a perspective view of the frame of FIG. 354;

FIG. 356 shows a top view of the frame of FIG. 355;

FIG. 357 shows a side view of the frame of FIG. 356;

FIG. 358 shows a perspective view of an example paddle frame for a device or implant;

FIG. 359 shows a front sectional view of the frame of FIG. 358;

FIG. 360 shows a front sectional view of an example paddle frame for a device or implant;

FIG. 361 shows a top view of the frame of FIG. 360;

FIG. 362 shows a front sectional view of an example paddle frame for a device or implant;

FIG. 363 shows a perspective view of a paddle frame attached to an elongated cap;

FIG. 364 shows a partial front view of the frame and elongated cap of FIG. 363;

FIG. 365 is a front view of an example of a connection mechanism between a rigid inner frame portion and a flexible outer frame portion of a paddle frame;

FIG. 366 is a perspective view of the paddle frame assembly of FIG. 365;

FIG. 367 is a top view of the paddle frame assembly of FIG. 365;

FIG. 368 is a side view of the paddle frame assembly of FIG. 365;

FIG. 369 is a rear view of the paddle frame assembly of FIG. 365;

FIG. 370 is a front view of an example of a connection mechanism between a rigid inner frame portion and a flexible outer frame portion of a paddle frame;

FIG. 371 is a side view of the paddle frame assembly of FIG. 370;

FIG. 372 is a rear view of the paddle frame assembly of FIG. 370;

FIG. 373 is a front view of an example of a connection between a rigid inner frame portion and a flexible outer frame portion of a paddle frame;

FIG. 374 is a side view of the paddle frame of FIG. 373;

FIG. 375 is a perspective view of the paddle frame of FIG. 373;

FIG. 376 is a front view of an example of a connection between a rigid inner frame portion and a flexible outer frame portion of a paddle frame;

FIG. 377 is a perspective view of the paddle frame assembly of FIG. 376;

FIG. 378 shows a schematic representation of an anchor portion of a device or implant in the closed position;

FIG. 379 shows a schematic representation of the anchor portion of FIG. 378 in the closed position with leaflets of a native valve secured by the anchor portion;

FIG. 380 shows a schematic representation of an example anchor portion for a device or implant in the closed position;

FIG. 381 shows a schematic representation of the anchor portion of FIG. 380 with leaflets of a native valve secured by the anchor portion;

FIG. 382 shows a schematic representation of the anchor portion of FIG. 380 in a partially open position;

FIG. 383 shows a schematic representation of the anchor portion of FIG. 380 in an open position;

FIG. 384 shows a plan view of an anchor of the anchor portion of FIG. 380 with the anchor laid flat;

FIG. 385 shows a schematic representation of an anchor of the anchor portion of FIG. 380 in the closed position and a clasp attached to the anchor;

FIG. 386 shows a schematic representation of the anchor and the clasp of FIG. 385 in the closed position with a leaflet of a native valve secured by the anchor and clasp;

FIG. 387 shows a schematic representation of the anchor and the clasp of FIG. 385 with the anchor in an open position and the clasp in a closed position;

FIG. 388 shows a schematic representation of an anchor portion and clasp of a device or implant in a closed position showing inward bias of outer paddles of the anchor portion;

FIG. 389 shows a schematic representation of one side of the anchor portion of FIG. 388 in a closed position showing inward bias of the outer paddle;

FIG. 390 shows a plan view of an example of a clasp for a device or implant with the clasp laid flat;

FIG. 391 shows an example of a clasp for a device or implant;

FIG. 392 shows a plan view of an example of a clasp for a device or implant with the clasp laid flat;

FIG. 393 shows a side view of an example of a clasp for a device or implant with the clasp in a closed position;

FIG. 394 shows the anchor portion of FIG. 388 positioned in a shape memory alloy jig;

FIG. 395 shows a perspective view of an example of an anchor portion for a device or implant;

FIG. 396 shows a plan view of an example of an inner member and an inner paddle portion of the anchor portion of FIG. 395;

FIG. 397 shows a left-side perspective view of an example of a device or implant;

FIG. 398 shows a right-side perspective view of the device of FIG. 397;

FIG. 399 shows a front view of the device of FIG. 397;

FIG. 400 shows a partial perspective view of the distal portion of the device of FIG. 397 where the anchor portion is attached to a cap at a distal end of the device;

FIG. 401 shows a perspective view of an example of a clasp for a device or implant;

FIG. 402 shows a perspective view of an example of a clasp for a device or implant;

FIG. 403 shows a perspective view of an example of a clasp for a device or implant;

FIG. 404 shows a perspective view of an example of a clasp for a device or implant;

FIG. 405 shows a perspective view of an example of a clasp for a device or implant;

FIG. 406 shows a perspective view of an example of a clasp for a device or implant;

FIG. 407A illustrates an example of a holding or locking mechanism;

FIG. 407B shows an example of the holding or locking mechanism of FIG. 407A deployed in housing;

FIG. 407C shows a cut-away view of FIG. 407B showing the holding or locking mechanism in the housing;

FIG. 408A shows an example of a cap engaged with a paddle;

FIG. 408B shows a close-up of the cap of FIG. 408A without the paddle;

FIG. 408C is a perspective view of the cap and the paddle shown in FIG. 408A;

FIG. 408D is a cross-sectional view that shows deflection of the paddle caused by various degrees of retraction of the paddle into the cap;

FIG. 408E is a perspective view that shows the degrees of deflection of FIG. 408D;

FIG. 408F is a schematic illustration that shows a configuration where the paddles are simultaneously deflected by coupled retraction into a cap;

FIG. 408G is a perspective view of a cap and the paddle assembly;

FIG. 409A is a top perspective view of an assembly of a cap and two independently adjustable the paddles;

FIG. 409B is a bottom perspective view of an assembly of FIG. 409A;

FIG. 409C is a cross-sectional view that illustrates independent control of the paddles FIGS. 409A and 409B;

FIG. 410A is a partial cross-sectional view of an adjustable the paddle assembly;

FIG. 410B is a perspective view of the adjustable the paddle assembly of FIG. 410A;

FIG. 410C is a sectional view of the adjustable the paddle assembly of FIG. 410B;

FIG. 410D is a sectional view of the adjustable the paddle assembly of FIG. 410B;

FIG. 410E is a side view of the adjustable the paddle assembly of FIG. 410B;

FIG. 410F is a side view of an adjustable the paddle assembly showing the paddles in a first actuation position.

FIG. 410G is a side view of an adjustable the paddle assembly showing the paddles in a second actuation position.

FIG. 410H is a side view of an adjustable the paddle assembly showing the paddles in a third actuation position.

FIG. 411A is a side perspective view of an adjustable the paddle assembly;

FIG. 411B is a side view of the adjustable the paddle assembly of FIG. 411A;

FIG. 411C is a front view of the adjustable the paddle assembly of FIG. 411A;

FIGS. 411D and 411E show use of the adjustable the paddle assembly of FIG. 411A in a valve repair device or implant;

FIG. 412A shows an example of a paddle structure made from sheet material;

FIG. 412B is a side view of the paddle structure of FIG. 412A;

FIG. 412C is a top view the paddle structure of FIG. 412A;

FIG. 412D is a bottom the paddle structure of FIG. 412A;

FIG. 412E is another side view the paddle structure of FIG. 412A;

FIG. 412F shows detail of an example of eyelets of the structure the paddle structure of FIG. 412A;

FIG. 412G is a top view of the flat material used to make the paddle structure of FIG. 412A;

FIG. 412H shows an example of a valve repair device or implant that includes the paddle structure of FIG. 412A in a fully retracted position.

FIG. 412I shows the valve repair device or implant of FIG. 412H with the paddle structure in a partially open position;

FIG. 412J shows the valve repair device or implant of FIG. 412H with the paddle structure in a laterally extended or open position.

FIG. 412K is a perspective view of a die that can be used to make the paddle structure of FIG. 412A;

FIG. 412L is a perspective view of the die illustrated by FIG. 412K;

FIGS. 413A and 413B shows an example of a valve repair device or implant with compressible outer the paddle portions;

FIG. 414A is a perspective view example of a valve repair device or implant with compressible outer the paddle portions;

FIG. 414B is a perspective view showing a paddle of the valve repair device or implant illustrated by FIG. 414A;

FIG. 415A is a side view of an example of a valve repair device or implant in an open condition with a gap filling material;

FIG. 415B is a view of the valve repair device or implant of FIG. 415A attached to the leaflets of a native valve as seen from a ventricular side of the native valve;

FIG. 415C is a side view of the valve repair device or implant of FIG. 415A in a closed condition;

FIG. 415D is a front view of the valve repair device or implant of FIG. 415A in a closed condition;

FIG. 416A is a side view of an example of a valve repair device or implant in an open condition with a gap filling material;

FIG. 416B is a view of the valve repair device or implant of FIG. 416A attached to the leaflets of a native valve as seen from a ventricular side of the native valve;

FIG. 416C is a side view of the valve repair device or implant of FIG. 416A in a closed condition; and

FIG. 416D is a front view of the valve repair device or implant of FIG. 416A in a closed condition;

FIG. 417 is a perspective view of an example coupling assembly for an example device in a coupled condition;

FIG. 418 is a front view of the coupling assembly of FIG. 417;

FIG. 419 is a perspective cross-section view of the coupling assembly of FIGS. 417-418 taken along the line 418-418 in FIG. 418;

FIG. 420 is a cross-section view of the coupling assembly of FIG. 418 taken along the line 418-418 in FIG. 418;

FIG. 421 is a perspective view of the example coupling assembly of FIG. 418 in an uncoupled condition;

FIG. 422 is a front view of the coupling assembly of FIG. 421;

FIG. 423 is a perspective cross-section view of the coupling assembly of FIGS. 423-424 taken along the line 422-422 in FIG. 422;

FIG. 424 is a cross-section view of the coupling assembly of FIG. 418 taken along the line 422-422 in FIG. 422;

FIG. 425 is a perspective view of an example coupling assembly for an example device in a coupled condition;

FIG. 426 is a front view of the coupling assembly of FIG. 425;

FIG. 427 is a perspective cross-section view of the coupling assembly of FIGS. 425-426 taken along the line 426-426 in FIG. 426;

FIG. 428 is a cross-section view of the coupling assembly of FIGS. 425-426 taken along the line 426-426 in FIG. 426;

FIG. 429 is a perspective view of the example coupling assembly of FIG. 425 in an uncoupled condition;

FIG. 430 is a front view of the coupling assembly of FIG. 429;

FIG. 431 is a perspective cross-section view of the coupling assembly of FIGS. 429-430 taken along the line 430-430 in FIG. 430;

FIG. 432 is a cross-section view of the coupling assembly of FIGS. 429-430 taken along the line 430-430 in FIG. 430;

FIG. 433 is a perspective view of an example coupling assembly for an example device in a coupled condition;

FIG. 434 is a front view of the coupling assembly of FIG. 433;

FIG. 435 is a perspective cross-section view of the coupling assembly of FIGS. 433-434 taken along the line 434-434 in FIG. 434;

FIG. 436 is a cross-section view of the coupling assembly of FIGS. 433-434 taken along the line 434-434 in FIG. 434;

FIG. 437 is a perspective view of the example coupling assembly of FIG. 433 in an uncoupled condition;

FIG. 438 is a front view of the coupling assembly of FIG. 437;

FIG. 439 is a perspective cross-section view of the coupling assembly of FIGS. 437-438 taken along the line 438-438 in FIG. 438;

FIG. 440 is a cross-section view of the coupling assembly of FIGS. 437-438 taken along the line 438-438 in FIG. 438;

FIG. 441 is a perspective view of an example coupling assembly for an example device in a coupled condition;

FIG. 442 is a front view of the coupling assembly of FIG. 441;

FIG. 443 is a perspective cross-section view of the coupling assembly of FIGS. 441-442 taken along the line 442-442 in FIG. 442;

FIG. 444 is a cross-section view of the coupling assembly of FIGS. 441-442 taken along the line 442-442 in FIG. 442;

FIG. 445 is a perspective view of the example coupling assembly of FIG. 441 in an uncoupled condition;

FIG. 446 is a front view of the coupling assembly of FIG. 445;

FIG. 447 is a perspective cross-section view of the coupling assembly of FIGS. 445-446 taken along the line 446-446 in FIG. 446;

FIG. 448 is a cross-section view of the coupling assembly of FIGS. 445-446 taken along the line 446-446 in FIG. 446;

FIG. 449 is a schematic view of an example coupling assembly for an example device in a coupled condition;

FIG. 450 is a schematic view of the example coupling assembly of FIG. 449 in an uncoupled condition;

FIG. 451 is a perspective view of an example coupling assembly for an example device in a coupled condition;

FIG. 452 is a front view of the coupling assembly of FIG. 451;

FIG. 453 is a perspective cross-section view of the coupling assembly of FIGS. 451-452 taken along the line 452-452 in FIG. 452;

FIG. 454 is a cross-section view of the coupling assembly of FIGS. 451-452 taken along the line 452-452 in FIG. 452;

FIG. 455 is a perspective view of the example coupling assembly of FIG. 451 in an uncoupled condition;

FIG. 456 is a front view of the coupling assembly of FIG. 455;

FIG. 457 is a perspective cross-section view of the coupling assembly of FIGS. 455-456 taken along the line 456-456 in FIG. 456;

FIG. 458 is a cross-section view of the coupling assembly of FIGS. 455-456 taken along the line 456-456 in FIG. 456;

FIG. 459 is a perspective view of an example coupling assembly for an example device in a coupled condition;

FIG. 460 is a front view of the coupling assembly of FIG. 459;

FIG. 461 is a perspective cross-section view of the coupling assembly of FIGS. 459-460 taken along the line 460-460 in FIG. 460;

FIG. 462 is a cross-section view of the coupling assembly of FIGS. 459-460 taken along the line 460-460 in FIG. 460;

FIG. 463 is a perspective view of the example coupling assembly of FIG. 459 in an uncoupled condition;

FIG. 464 is a front view of the coupling assembly of FIG. 463;

FIG. 465 is a perspective cross-section view of the coupling assembly of FIGS. 463-464 taken along the line 464-464 in FIG. 464;

FIG. 466 is a cross-section view of the coupling assembly of FIGS. 463-464 taken along the line 464-464 in FIG. 464;

FIG. 467 is a perspective view of an example coupling assembly for an example device in a coupled condition;

FIG. 468 is a front view of the coupling assembly of FIG. 467;

FIG. 469 is a perspective cross-section view of the coupling assembly of FIGS. 467-468 taken along the line 468-468 in FIG. 468;

FIG. 470 is a cross-section view of the coupling assembly of FIGS. 467-468 taken along the line 468-468 in FIG. 468;

FIG. 471 is a perspective view of the example coupling assembly of FIG. 467 in an uncoupled condition;

FIG. 472 is a front view of the coupling assembly of FIG. 471;

FIG. 473 is a perspective cross-section view of the coupling assembly of FIGS. 471-472 taken along the line 472-472 in FIG. 472;

FIG. 474 is a cross-section view of the coupling assembly of FIGS. 471-472 taken along the line 472-472 in FIG. 472;

FIG. 475 is a perspective view of an example coupling assembly for an example device in a coupled condition;

FIG. 476 is a front view of the coupling assembly of FIG. 475;

FIG. 477 is a perspective cross-section view of the coupling assembly of FIGS. 475-476 taken along the line 476-476 in FIG. 476;

FIG. 478 is a cross-section view of the coupling assembly of FIGS. 475-476 taken along the line 476-476 in FIG. 476;

FIG. 479 is a perspective view of the example coupling assembly of FIG. 475 in an uncoupled condition;

FIG. 480 is a front view of the coupling assembly of FIG. 479;

FIG. 481 is a perspective cross-section view of the coupling assembly of FIGS. 479-480 taken along the line 480-480 in FIG. 480;

FIG. 482 is a cross-section view of the coupling assembly of FIGS. 479-480 taken along the line 480-480 in FIG. 480;

FIG. 483 is a perspective view of an example device;

FIG. 484 is a front view of the device of FIG. 483;

FIG. 485 is a perspective cross-section view of the device of FIGS. 483-484 taken along the line 484-484 in FIG. 484 and showing an example actuation mechanism in a moveable condition;

FIG. 486 is a cross-section view of the device of FIGS. 483-484 taken along the line 484-484 in FIG. 484;

FIG. 487 is an enlarged detail view of the area 485 of FIG. 485;

FIG. 488 is an enlarged detail view of the area 486 of FIG. 486;

FIG. 489 is a perspective cross-section view of the device of FIGS. 483-484 taken along the line 484-484 in FIG. 484 and showing the actuation mechanism in a latched condition;

FIG. 490 is a cross-section view of the device of FIGS. 483-484 taken along the line 484-484 in FIG. 484;

FIG. 491 is an enlarged detail view of the area 489 of FIG. 489;

FIG. 492 is an enlarged detail view of the area 490 of FIG. 490;

FIG. 493 is a perspective view of an example actuation mechanism of the device of FIGS. 483-484;

FIG. 494 is a front view of the mechanism of FIG. 494;

FIG. 495 is a perspective cross-section view of the mechanism of FIG. 493 taken along the line 494-494 of FIG. 494;

FIG. 496 is an enlarged detail view of the area 495 of FIG. 495;

FIG. 497 is a perspective view of a latch tube of the device of FIGS. 483-484;

FIG. 498 is a perspective view of an example device;

FIG. 499 is a front view of the device of FIG. 499;

FIG. 500 is a perspective cross-section view of the device of FIGS. 498-499 taken along the line 499-499 in FIG. 499 and showing an example actuation mechanism in a moveable condition;

FIG. 501 is a cross-section view of the device of FIGS. 498-499 taken along the line 499-499 in FIG. 499;

FIG. 502 is an enlarged detail view of the area 500 of FIG. 500;

FIG. 503 is an enlarged detail view of the area 501 of FIG. 501;

FIG. 504 is a perspective cross-section view of the device of FIGS. 498-499 taken along the line 499-499 in FIG. 499 and showing the actuation mechanism in a latched condition;

FIG. 505 is a cross-section view of the device of FIGS. 498-499 taken along the line 499-499 in FIG. 499;

FIG. 506 is an enlarged detail view of the area 504 of FIG. 504;

FIG. 507 is an enlarged detail view of the area 505 of FIG. 505;

FIG. 508 is a perspective view of an example actuation mechanism of the device of FIGS. 498-499;

FIG. 509 is a front view of the mechanism of FIG. 508;

FIG. 510 is a perspective cross-section view of the mechanism of FIG. 508 taken along the line 509-509 of FIG. 509;

FIG. 511 is an enlarged detail view of the area 510 of FIG. 510;

FIG. 512 is a perspective view of a latch tube of the device of FIGS. 498-499;

FIG. 513 is a front view of an attachment portion of the actuation mechanism of FIG. 508 in a coupled condition;

FIG. 514 is a front view of the attachment portion of FIG. 513 in a decoupled condition;

FIG. 515 is a cross-section view of the attachment portion of FIG. 513;

FIG. 516 is a cross-section view of the attachment portion of FIG. 514;

FIG. 517 is a perspective view of an example device;

FIG. 518 is a front view of the device of FIG. 517;

FIG. 519 is a perspective cross-section view of the device of FIGS. 517-518 taken along the line 518-518 in FIG. 518 and showing an example actuation mechanism in a moveable condition;

FIG. 520 is a cross-section view of the device of FIGS. 517-518 taken along the line 518-518 in FIG. 518;

FIG. 521 is an enlarged detail view of the area 519 of FIG. 519;

FIG. 522 is an enlarged detail view of the area 520 of FIG. 520;

FIG. 523 is a perspective cross-section view of the device of FIGS. 517-518 taken along the line 518-518 in FIG. 518 and showing the actuation mechanism in a latched condition;

FIG. 524 is a cross-section view of the device of FIGS. 517-518 taken along the line 518-518 in FIG. 518;

FIG. 525 is an enlarged detail view of the area 523 of FIG. 523;

FIG. 526 is an enlarged detail view of the area 524 of FIG. 524;

FIG. 527 is a perspective view of an example actuation mechanism of the device of FIGS. 517-518 in a moveable condition;

FIG. 528 is a front view of the actuation mechanism of FIG. 527;

FIG. 529 is a cross-section view of the actuation mechanism of FIG. 527 taken along the line 528-528 of FIG. 528;

FIG. 530 is an enlarged detail view of the area 529 of FIG. 529;

FIG. 531 is a perspective view of an example actuation mechanism of the device of FIGS. 517-518 in a latched condition;

FIG. 532 is a front view of the actuation mechanism of FIG. 531;

FIG. 533 is a cross-section view of the actuation mechanism of FIG. 531 taken along the line 532-532 of FIG. 532;

FIG. 534 is an enlarged detail view of the area 533 of FIG. 533;

FIG. 535 is a perspective view of an example handle release mechanism of an example delivery system for an example device;

FIG. 536 is a side view of the handle release mechanism of FIG. 535;

FIG. 537 is a front view of the handle release mechanism of FIG. 535;

FIG. 538 is a perspective cross-section view of the handle release mechanism of FIG. 535 taken along the line 537-537 of FIG. 537;

FIG. 539 is a cross-section view of the handle release mechanism of FIG. 535 taken along the line 537-537 of FIG. 537;

FIG. 540 is a perspective view of the example handle release mechanism of FIG. 535 with a slide in a retracted condition;

FIG. 541 is a side view of the handle release mechanism of FIG. 540;

FIG. 542 is a front view of the handle release mechanism of FIG. 540;

FIG. 543 is a perspective cross-section view of the handle release mechanism of FIG. 540 taken along the line 542-542 of FIG. 542;

FIG. 544 is a cross-section view of the handle release mechanism of FIG. 540 taken along the line 542-542 of FIG. 542;

FIG. 545 is a perspective view of the example handle release mechanism of FIG. 535 with the slide retracted and a latch member of an outer body disengaged from an inner body;

FIG. 546 is a side view of the handle release mechanism of FIG. 545;

FIG. 547 is a front view of the handle release mechanism of FIG. 545;

FIG. 548 is a perspective cross-section view of the handle release mechanism of FIG. 545 taken along the line 547-547 of FIG. 547;

FIG. 549 is a cross-section view of the handle release mechanism of FIG. 545 taken along the line 547-547 of FIG. 547;

FIG. 550 is a perspective view of the example handle release mechanism of FIG. 535 with the slide retracted and finger members of the inner body disengaged from the connector body;

FIG. 551 is a side view of the handle release mechanism of FIG. 550;

FIG. 552 is a front view of the handle release mechanism of FIG. 550;

FIG. 553 is a perspective cross-section view of the handle release mechanism of FIG. 550 taken along the line 552-552 of FIG. 552;

FIG. 554 is a cross-section view of the handle release mechanism of FIG. 550 taken along the line 552-552 of FIG. 552;

FIG. 555 is a perspective view of the example handle release mechanism of FIG. 535 with the inner and outer body disengaged from the connector body;

FIG. 556 is a side view of the handle release mechanism of FIG. 555;

FIG. 557 is a front view of the handle release mechanism of FIG. 555;

FIG. 558 is a perspective cross-section view of the handle release mechanism of FIG. 555 taken along the line 557-557 of FIG. 557;

FIG. 559 is a cross-section view of the handle release mechanism of FIG. 555 taken along the line 557-557 of FIG. 557;

FIG. 560 is a perspective view of an example handle release mechanism of an example delivery system for an example device;

FIG. 561 is a side view of the handle release mechanism of FIG. 560;

FIG. 562 is a front view of the handle release mechanism of FIG. 560;

FIG. 563 is a perspective cross-section view of the handle release mechanism of FIG. 560 taken along the line 562-562 of FIG. 562;

FIG. 564 is a cross-section view of the handle release mechanism of FIG. 560 taken along the line 562-562 of FIG. 562;

FIG. 565 is a perspective view of the example handle release mechanism of FIG. 560 with a slide in a retracted condition;

FIG. 566 is a side view of the handle release mechanism of FIG. 565;

FIG. 567 is a front view of the handle release mechanism of FIG. 565;

FIG. 568 is a perspective cross-section view of the handle release mechanism of FIG. 565 taken along the line 567-567 of FIG. 567;

FIG. 569 is a cross-section view of the handle release mechanism of FIG. 565 taken along the line 567-567 of FIG. 567;

FIG. 570 is a perspective view of the example handle release mechanism of FIG. 560 with the slide in a retracted condition and an outer body disengaged from a connector body;

FIG. 571 is a side view of the handle release mechanism of FIG. 570;

FIG. 572 is a front view of the handle release mechanism of FIG. 570;

FIG. 573 is a perspective cross-section view of the handle release mechanism of FIG. 570 taken along the line 572-572 of FIG. 572;

FIG. 574 is a cross-section view of the handle release mechanism of FIG. 570 taken along the line 572-572 of FIG. 572;

FIG. 575 is a schematic illustration of an example clasp for a device;

FIG. 576 is a schematic illustration of the clasp of FIG. 575 including an optional retention surface;

FIG. 577 is a schematic illustration of an example clasp for a device;

FIG. 578 is a schematic illustration of the clasp of FIG. 577 including an optional retention surface;

FIG. 579 is a schematic illustration of an example clasp for a device;

FIG. 580 is a schematic illustration of the clasp of FIG. 579 including an optional retention surface;

FIG. 581 is a schematic illustration of an example clasp for a device;

FIG. 582 is a schematic illustration of the clasp of FIG. 581 including an optional retention surface;

FIG. 583 is a schematic illustration of an example braided spacer in an unexpanded condition;

FIG. 584 is a schematic illustration of an example braided spacer in an expanded condition;

FIG. 585 shows a perspective view of an example expandable coaptation element in an expanded condition;

FIG. 586 shows a front view of the example expandable coaptation element of FIG. 585;

FIG. 587 shows a perspective cross-section view of the example expandable coaptation element of FIG. 585;

FIG. 588 shows a cross-section view of the example expandable coaptation element of FIG. 585;

FIG. 589 shows a perspective view of an example expandable coaptation element in an unexpanded condition;

FIG. 590 shows a front view of the expandable coaptation element of FIG. 589;

FIG. 591 shows a perspective cross-section view of the example expandable coaptation element of FIG. 589 taken along the line 590-590 of FIG. 590;

FIG. 592 shows a cross-section view of the example expandable coaptation element of FIG. 589 taken along the line 590-590 of FIG. 590;

FIG. 593 shows a perspective view of the example expandable coaptation element of FIG. 585 in an expanded condition;

FIG. 594 shows a front view of the expandable coaptation element of FIG. 593;

FIG. 595 shows a perspective cross-section view of the example expandable coaptation element of FIG. 593 taken along the line 594-594 of FIG. 594;

FIG. 596 shows a cross-section view of the example expandable coaptation element of FIG. 593 taken along the line 594-594 of FIG. 594;

FIG. 597 shows a perspective view of an example expandable coaptation element in an expanded condition;

FIG. 598 shows a front view of the expandable coaptation element of FIG. 597;

FIG. 599 shows a perspective cross-section view of the example expandable coaptation element of FIG. 597 taken along the line 598-598 of FIG. 598;

FIG. 600 shows a cross-section view of the example expandable coaptation element of FIG. 597 taken along the line 598-598 of FIG. 598;

FIG. 601 shows a perspective view of an example expandable coaptation element in an expanded condition;

FIG. 602 shows a front view of the expandable coaptation element of FIG. 601;

FIG. 603 shows a perspective cross-section view of the example expandable coaptation element of FIG. 601 taken along the line 602-602 of FIG. 602;

FIG. 604 shows a cross-section view of the example expandable coaptation element of FIG. 601 taken along the line 602-602 of FIG. 602;

FIG. 605 shows a perspective view of an example expandable coaptation element in an unexpanded condition with a latch tube in an unlatched condition;

FIG. 606 shows a front view of the expandable coaptation element of FIG. 605;

FIG. 607 shows a perspective cross-section view of the example expandable coaptation element of FIG. 605 taken along the line 606-606 of FIG. 606;

FIG. 608 shows a cross-section view of the example expandable coaptation element of FIG. 605 taken along the line 606-606 of FIG. 606;

FIG. 609 shows a perspective view of the example expandable coaptation element of FIG. 605 with the latch tube in a latched condition;

FIG. 610 shows a front view of the expandable coaptation element of FIG. 609;

FIG. 611 shows a perspective cross-section view of the example expandable coaptation element of FIG. 609 taken along the line 610-610 of FIG. 610;

FIG. 612 shows a cross-section view of the example expandable coaptation element of FIG. 609 taken along the line 610-610 of FIG. 610;

FIG. 613 shows an enlarged detailed view of the example expandable coaptation element of FIG. 605 taken in area 607 of FIG. 607;

FIG. 614 shows an enlarged detailed view of the example expandable coaptation element of FIG. 609 taken in area 611 of FIG. 611;

FIG. 615 shows a perspective view of an example expandable coaptation element in an unexpanded condition with a latch tube in an unlatched condition;

FIG. 616 shows a front view of the expandable coaptation element of FIG. 615;

FIG. 617 shows a perspective cross-section view of the example expandable coaptation element of FIG. 615 taken along the line 616-616 of FIG. 616;

FIG. 618 shows a cross-section view of the example expandable coaptation element of FIG. 615 taken along the line 616-616 of FIG. 616;

FIG. 619 shows a perspective view of the example expandable coaptation element of FIG. 615 with the latch tube in a latched condition;

FIG. 620 shows a front view of the expandable coaptation element of FIG. 619;

FIG. 621 shows a perspective cross-section view of the example expandable coaptation element of FIG. 619 taken along the line 620-620 of FIG. 620;

FIG. 622 shows a cross-section view of the example expandable coaptation element of FIG. 619 taken along the line 620-620 of FIG. 620;

FIG. 623 shows an enlarged detailed view of the example expandable coaptation element of FIG. 615 taken in area 617 of FIG. 617;

FIG. 624 shows an enlarged detailed view of the example expandable coaptation element of FIG. 619 taken in area 621 of FIG. 621;

FIG. 625 shows a perspective view of an example expandable coaptation element in an unexpanded condition;

FIG. 626 shows a top view of the expandable coaptation element of FIG. 625;

FIG. 627 shows a side view of the expandable coaptation element of FIG. 625;

FIG. 628 shows a front view of the expandable coaptation element of FIG. 625;

FIG. 629 shows a perspective view of the example expandable coaptation element of FIG. 625 in an expanded condition;

FIG. 630 shows a top view of the expandable coaptation element of FIG. 629;

FIG. 631 shows a side view of the expandable coaptation element of FIG. 629;

FIG. 632 shows a front view of the expandable coaptation element of FIG. 629;

FIG. 633 shows a perspective view of an example expandable coaptation element in an unexpanded condition;

FIG. 634 shows a top view of the expandable coaptation element of FIG. 633;

FIG. 635 shows a side view of the expandable coaptation element of FIG. 633;

FIG. 636 shows a front view of the expandable coaptation element of FIG. 633;

FIG. 637 shows a perspective view of the example expandable coaptation element of FIG. 633 in an expanded condition;

FIG. 638 shows a top view of the expandable coaptation element of FIG. 637;

FIG. 639 shows a side view of the expandable coaptation element of FIG. 637;

FIG. 640 shows a front view of the expandable coaptation element of FIG. 637;

FIG. 641 shows a perspective view of an example coupler for an example device with the coupler in a coupled condition;

FIG. 642 shows a front view of the example coupler of FIG. 641;

FIG. 643 shows a perspective cross-section view of the example coupler of FIG. 641 taken along the line 642-642 of FIG. 642;

FIG. 644 shows a cross-section view of the example expandable coaptation element of FIG. 641 taken along the line 642-642 of FIG. 642;

FIG. 645 shows a perspective view of the example coupler of FIG. 641 with a retention member retracted;

FIG. 646 shows a front view of the example coupler of FIG. 645;

FIG. 647 shows a perspective cross-section view of the example coupler of FIG. 645 taken along the line 646-646 of FIG. 646;

FIG. 648 shows a cross-section view of the example expandable coaptation element of FIG. 645 taken along the line 646-646 of FIG. 646;

FIG. 649 shows a perspective view of the example coupler of FIG. 641 with the retention member retracted and the coupler in an uncoupled condition;

FIG. 650 shows a front view of the example coupler of FIG. 649;

FIG. 651 shows a perspective cross-section view of the example coupler of FIG. 649 taken along the line 650-650 of FIG. 650;

FIG. 652 shows a cross-section view of the example expandable coaptation element of FIG. 649 taken along the line 650-650 of FIG. 650;

FIG. 653 shows a cross-section view of an expandable coaptation element in an unexpanded condition;

FIG. 654 shows a cross-section view of the expandable coaptation element of FIG. 653 in an expanded condition;

FIG. 655 shows a schematic view of an expandable coaptation element with a single expandable member;

FIG. 656 shows a schematic view of an expandable coaptation element with three expandable members;

FIG. 657 shows a schematic view of an expandable coaptation element with four expandable members;

FIG. 658 shows a schematic view of an expandable coaptation element with six expandable members arranged in two sets of three expandable members that are longitudinally spaced apart from each other;

FIG. 659 shows a perspective view of an implantation of an expandable coaptation element with and inner tube and an outer tube and an expandable coaptation member in an unexpanded condition;

FIG. 660 shows a top view thereof;

FIG. 661 shows a perspective view of the expandable coaptation element of FIG. 659 in an expanded condition;

FIG. 662 shows a top view thereof;

FIG. 663 shows an exploded view of the expandable coaptation element of FIG. 659;

FIG. 664 shows a flattened view of the portion of the outer tube that is cut to form an expandable component.

FIG. 665 shows a schematic view of a device with an expandable coaptation element being implanted into a patient's heart;

FIG. 666 shows a schematic view of a device with an expandable coaptation element being implanted into a patient's heart;

FIG. 667 shows an expandable coaptation element in the unexpanded condition;

FIG. 668 shows the expandable coaptation element of FIG. 667 in an expanded condition;

FIG. 669 shows the expandable coaptation element of FIG. 667 being expanded by the introduction of an elongated filling element;

FIG. 670 shows the elongated filling element filling the expandable coaptation element; and

FIG. 671 shows the expandable coaptation element of FIG. 670 with a second elongated filling element; and

FIG. 672 shows the expandable coaptation element of FIG. 670 with a plurality of pieces of filling material.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc. As used herein the term “simulation” encompasses simulations performed on a cadaver, a computer simulator, an imaginary person, in open space etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

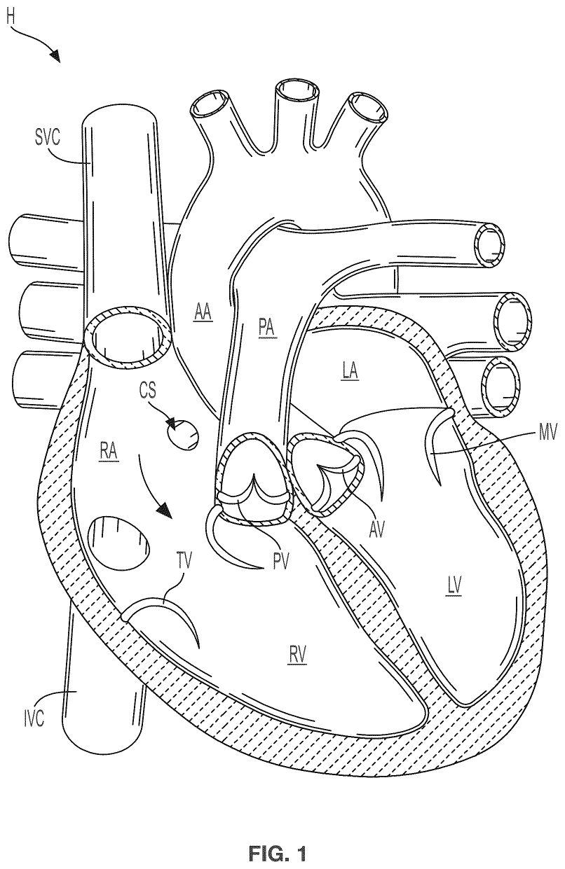

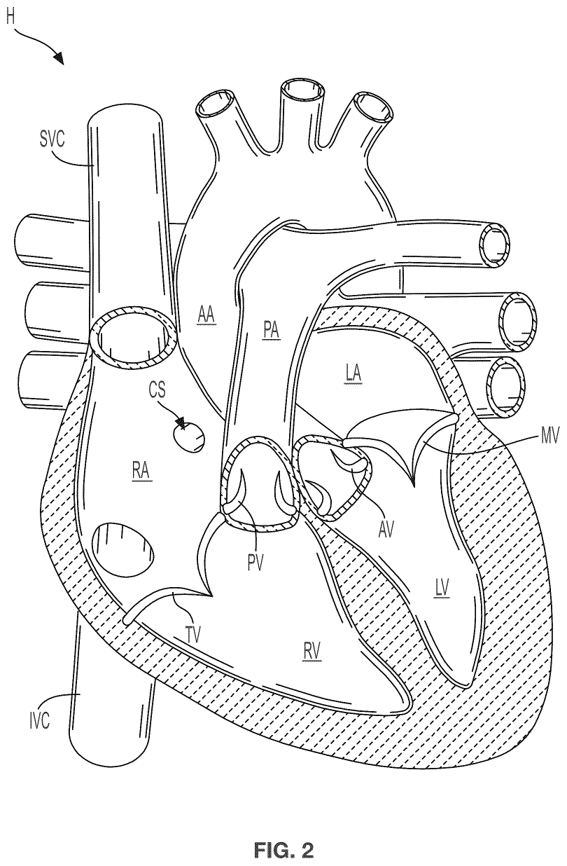

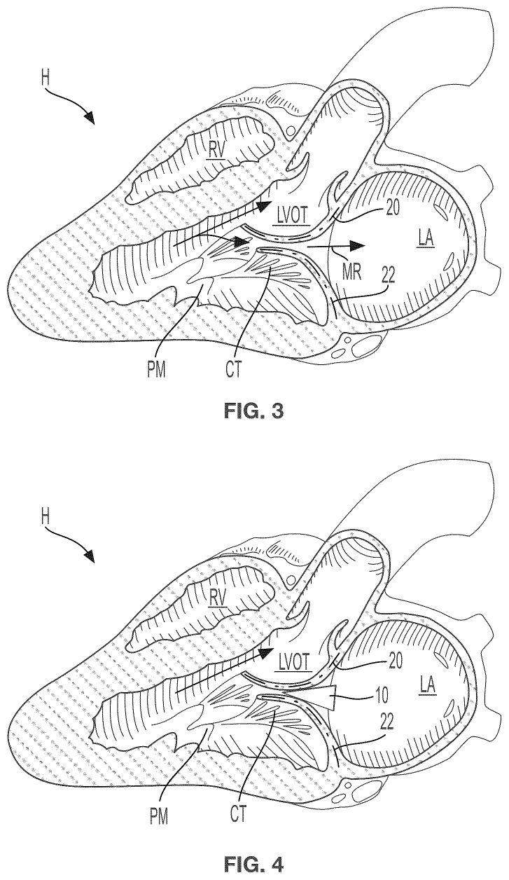

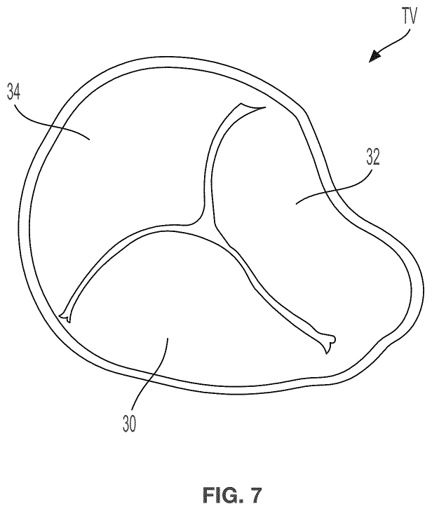

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit, or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

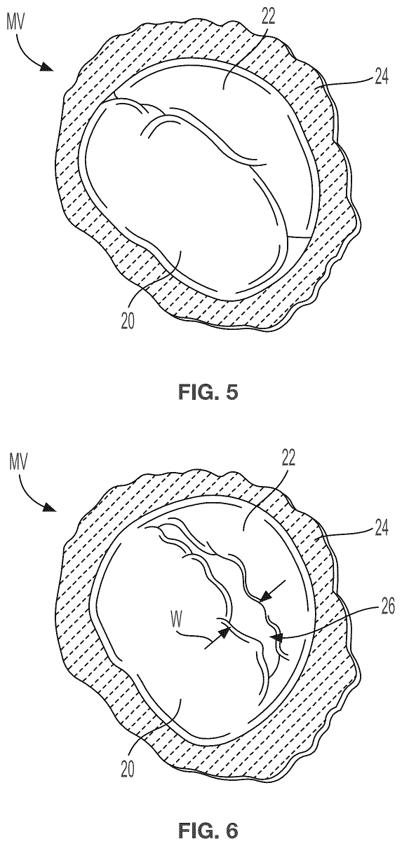

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, and balloon etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaptation element—e.g., spacer, coaption element, gap filler, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example device or implant can optionally have a coaptation element (e.g., spacer, coaption element, gap filler, etc.) and at least one anchor (e.g., one, two, three, or more). In some implementations, a device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing, preventing, or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The optional coaptation element (e.g., spacer, coaption element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft, rod, tube, wire, etc., to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

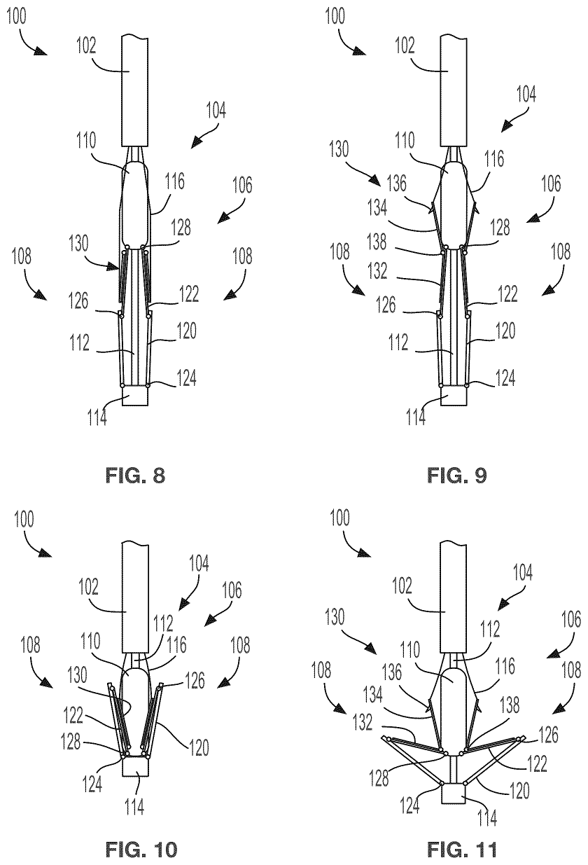

Referring now to FIGS. 8-15, a schematically illustrated device or implant 100 (e.g., an implantable prosthetic device, a prosthetic spacer device, a valve repair device, an implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features of devices or implants discussed elsewhere in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion/coaptation region 104 and an anchor portion/anchor region 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a movable arm 134, optional friction-enhancing elements, or other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate a coaptation element 110. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional friction-enhancing elements, or securing structures 136 (e.g., barbs, ridges, points, etc.).

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs, friction-enhancing elements, or securing structures 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, gap filler, etc.). The clasps 130 can be used to grasp and/or further secure the native leaflets by engaging the leaflets with optional barbs, friction-enhancing elements, or securing structures 136 and pinching the leaflets between the movable and fixed arms 134, 132. The optional barbs, friction-enhancing elements, or other securing structures 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system 102, so that the optional barbs, friction-enhancing elements, or other securing structures 136 (FIG. 9) do not catch or damage the delivery system 102. The actuation lines 116 can extend and attach to the movable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and movable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. To move the device 100 from the elongated condition to the shortened condition, the actuation element 112 is retracted to pull the cap 114 towards the coaptation element 110 (e.g., towards a spacer). The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

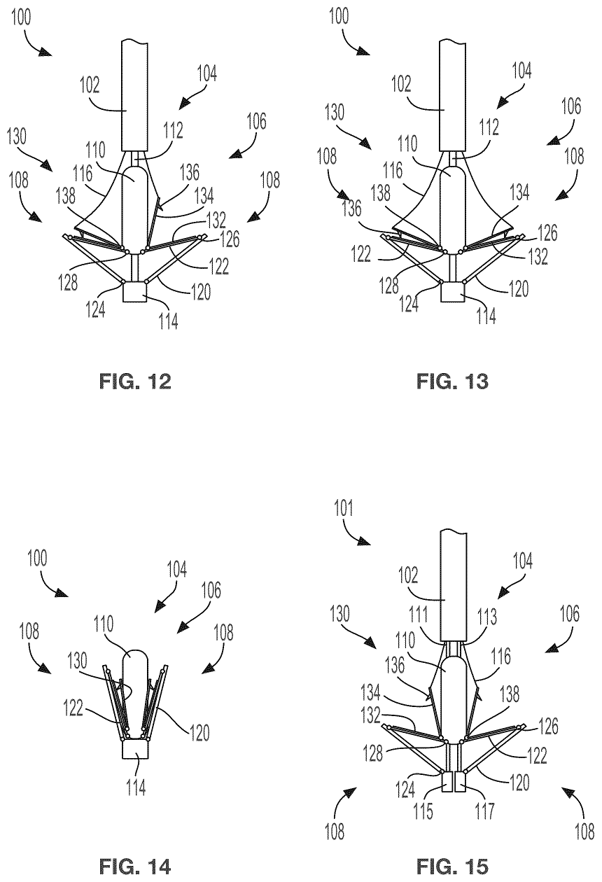

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder—and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements (e.g., actuation shafts, actuation rods, actuation tubes, actuation wires, etc.) 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

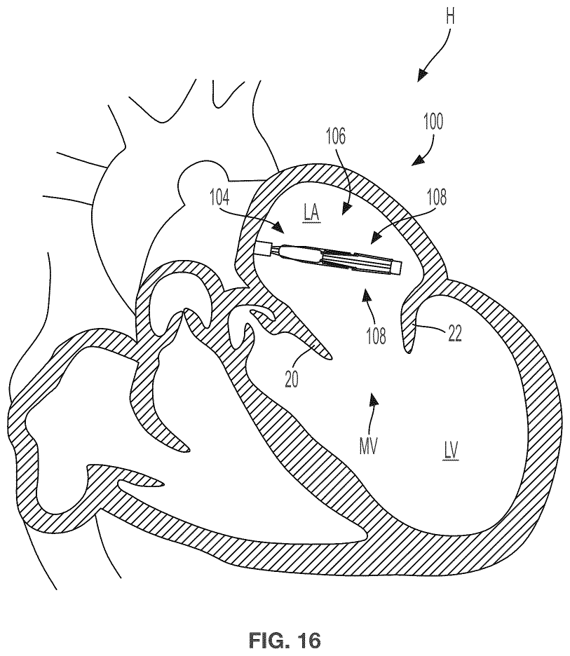

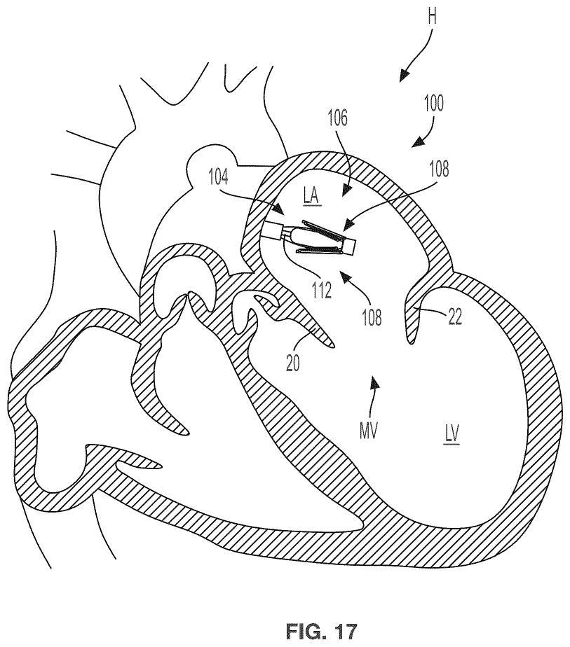

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

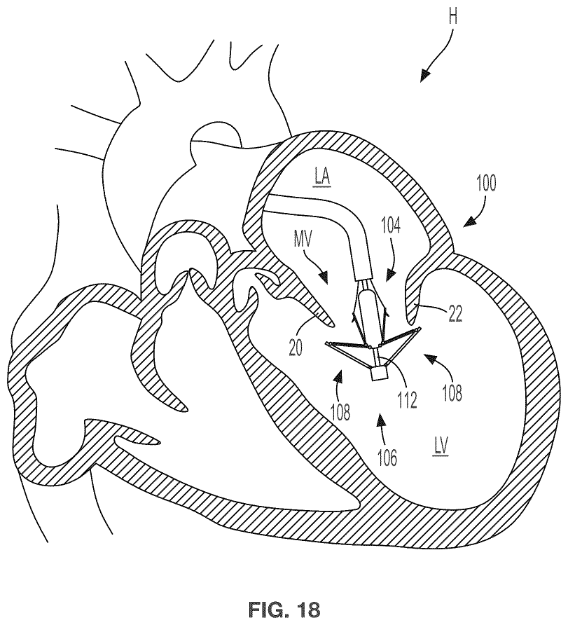

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

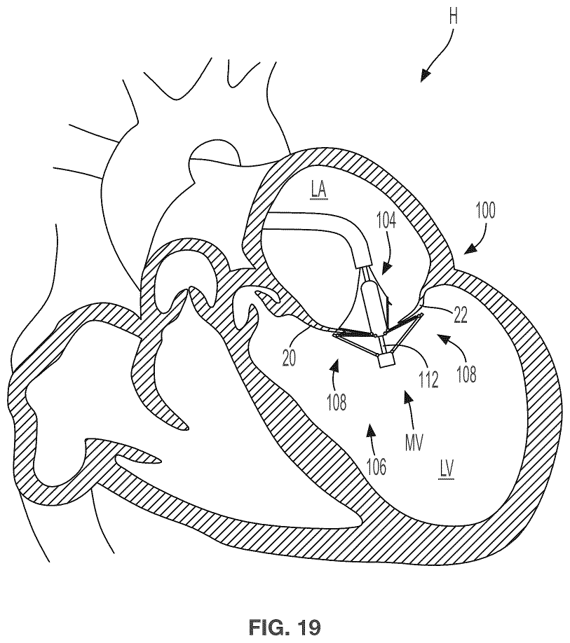

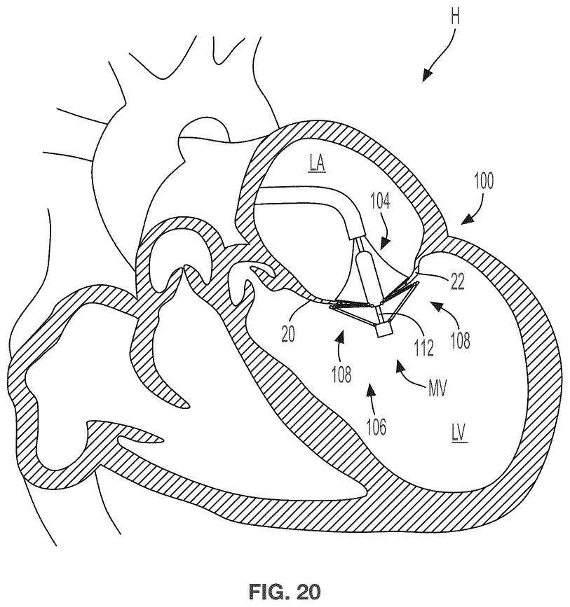

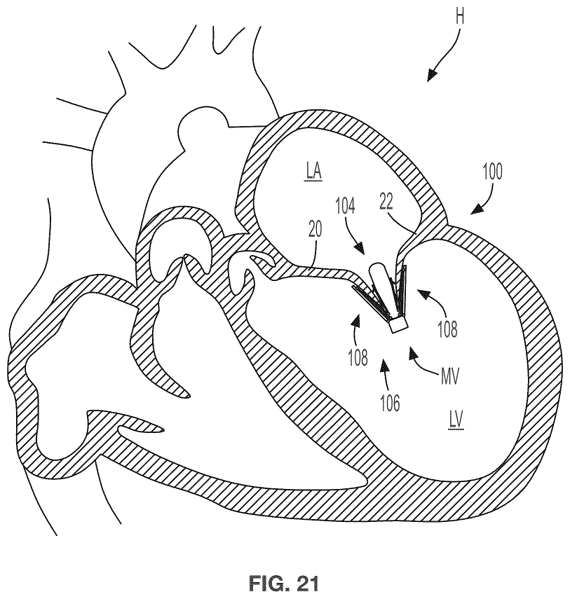

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

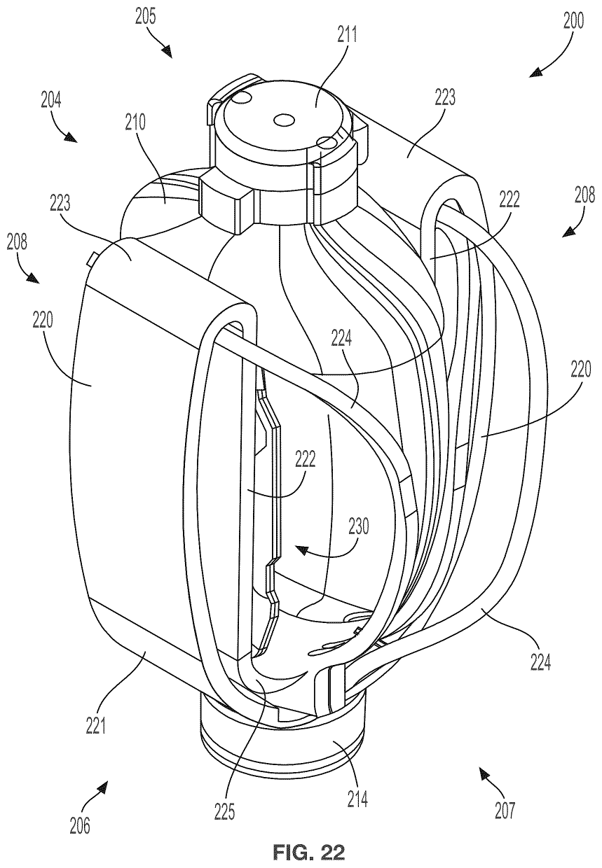

Any of the features disclosed by the present application can be used in a wide variety of different valve repair devices. FIGS. 22-27 and 56A-56H illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 22-27 and 56A-56H.

Referring now to FIG. 22, an example of a device or implant 200 is shown. The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable valve repair system (e.g., any valve repair system disclosed in the present application). The device/implant 200 can be a valve repair device, implantable device, or another type of implant that attaches to leaflets of a native valve.

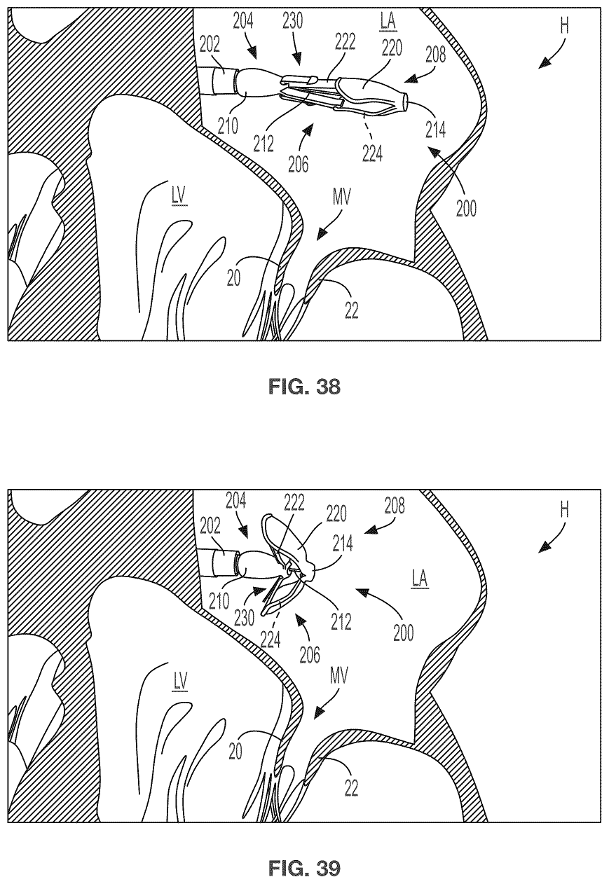

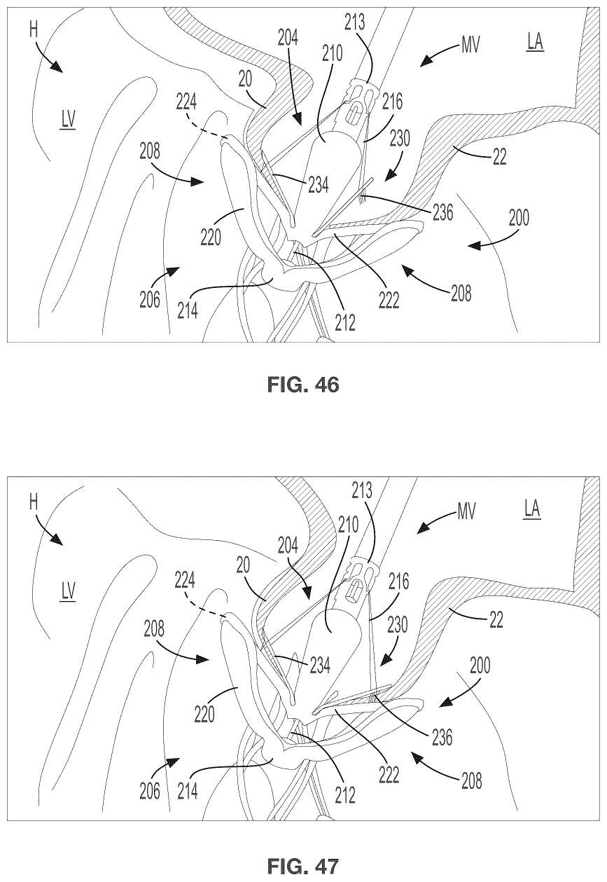

In some implementations, the device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism 213 (see e.g., FIGS. 43-49) of a delivery system 202 (see e.g., FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

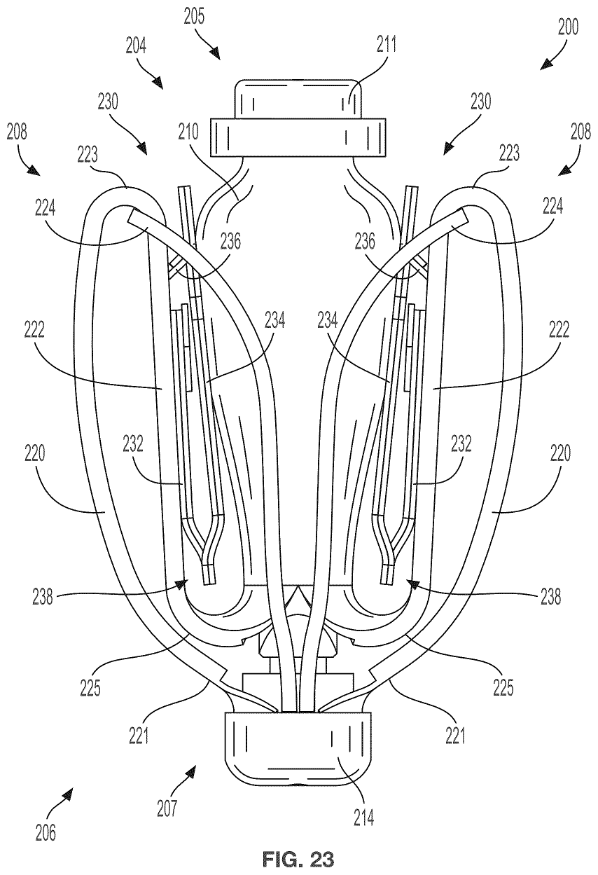

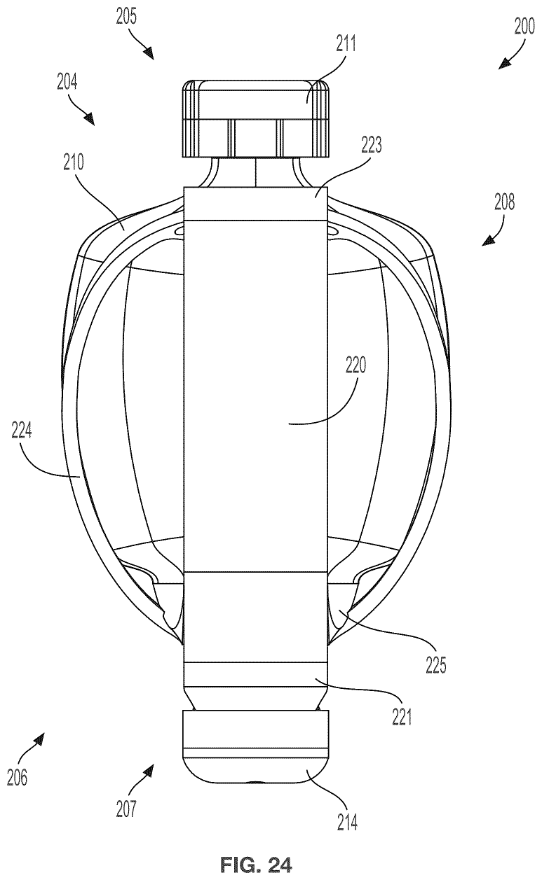

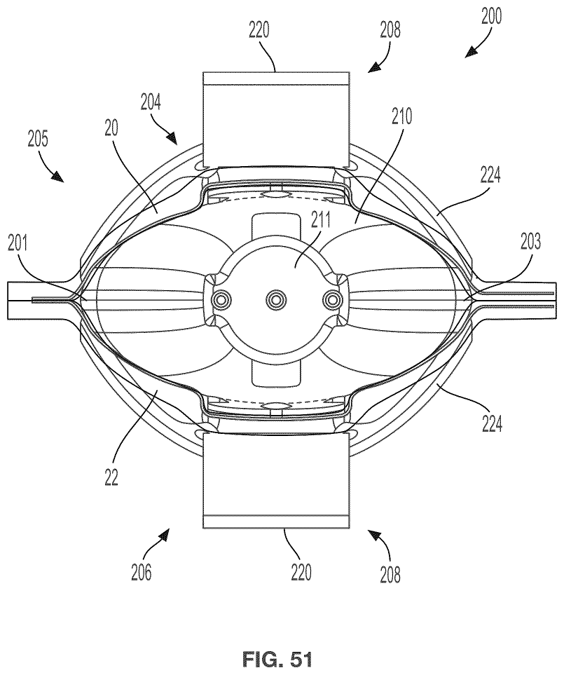

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., rod, plate, etc.) or a fixed portion 232 of the clasps 230. The stiffening of the inner paddle allows the device to move to the various different positions shown and described herein. The inner paddle 222, the outer paddle 220, and the coaptation element can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the device or implant 200, such that the native leaflets coapt entirely around the coaptation member or coaptation element 210. This can, for example, improve sealing of the native leaflets 20, 22 and thus prevent, inhibit, or further reduce mitral regurgitation.

In some implementations the clasps comprise a movable arm coupled to the anchors. In some implementations, the clasps 230 include a base or fixed arm 232, a movable arm 234, with optional barbs, friction-enhancing elements, or securing structures 236, and a joint portion 238. The fixed arms 232 are attached to the inner paddles 222, with the joint portion 238 disposed proximate the coaptation element 210. The joint portion 238 is spring-loaded so that the fixed and movable arms 232, 234 are biased toward each other when the clasp 230 is in a closed condition. In some implementations, the clasps 230 include friction-enhancing elements or means for securing, such as barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the movable arms 234 are opened to open the clasps 230 and expose the optional barbs, friction-enhancing elements, or securing structures 236. The clasps 230 are opened by applying tension to actuation lines 216 (e.g., as shown in FIGS. 43-48) attached to holes 235 in the movable arms 234, thereby causing the movable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the movable and fixed arms 232, 234 of the clasp 230. The tissue of the leaflet 20, 22 is not pierced by the optional barbs, friction-enhancing elements, or securing structures 236, though in some implementations the optional barbs 236 can partially or fully pierce through the leaflet 20, 22. The angle and height of the optional barbs, friction-enhancing elements or securing structures 236 relative to the movable arm 234 helps to secure the leaflet 20, 22 within the clasp 230. In particular, a force pulling the implant off of the native leaflet 20, 22 will encourage the optional barbs, friction-enhancing elements, or securing structures 236 to further engage the tissue, thereby ensuring better retention. Retention of the leaflet 20, 22 in the clasp 230 is further improved by the position of fixed arm 232 near the optional barbs, friction-enhancing elements, or securing structures 236 when the clasp 230 is closed. In this arrangement, the tissue is formed by the fixed arms 232 and the movable arms 234 and the optional barbs, friction-enhancing elements, or securing structures 236 into an S-shaped torturous path. Thus, forces pulling the leaflet 20, 22 away from the clasp 230 will encourage the tissue to further engage the optional barbs, friction-enhancing elements, or securing structures 236 before the leaflets 20, 22 can escape. For example, leaflet tension during diastole can encourage the optional barbs, friction-enhancing elements, or securing structures 236 to pull toward the end portion of the leaflet 20, 22. Thus, the S-shaped path can utilize the leaflet tension during diastole to engage the leaflets 20, 22 more tightly with the optional barbs, friction-enhancing elements or securing structures 236.

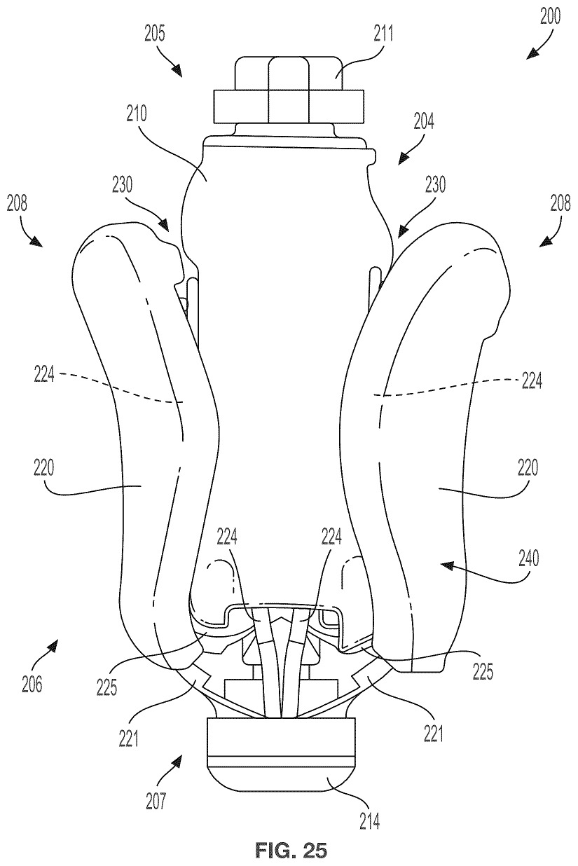

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent, inhibit or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the device or implant 200.

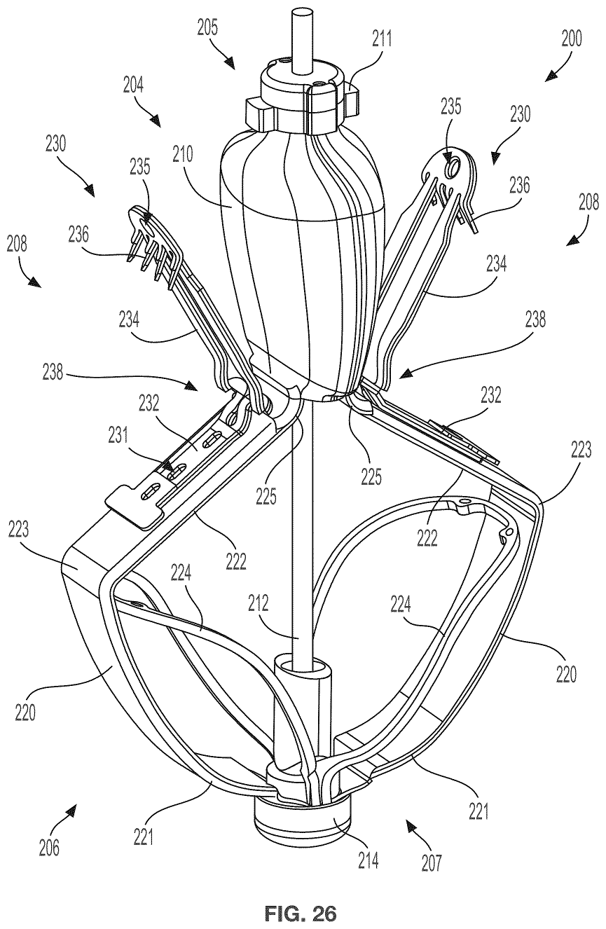

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 222 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 100 illustrated in FIG. 15).

In some implementations, the clasps 230 further secure the native leaflets 20, 22 by engaging the leaflets 20, 22 with optional barbs, friction-enhancing elements, or securing structures 236 and/or pinching the leaflets 20, 22 between the movable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to grip the leaflets 20, 22 more securely when the device 200 is closed (e.g., as can be seen in FIG. 51). The curved shape and rounded edges of the outer paddle 220 also prohibits or inhibits tearing of the leaflet tissue.

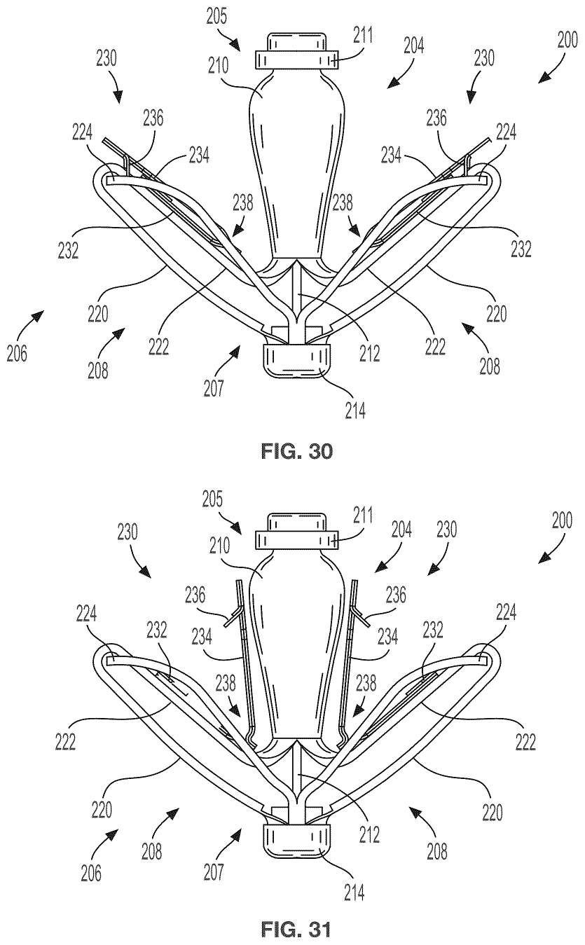

Referring now to FIGS. 30-37, the device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and movable arms 232, 234 of the clasp 230 is limited to the extent that the inner paddle 222 has spread away from the coaptation element 210.

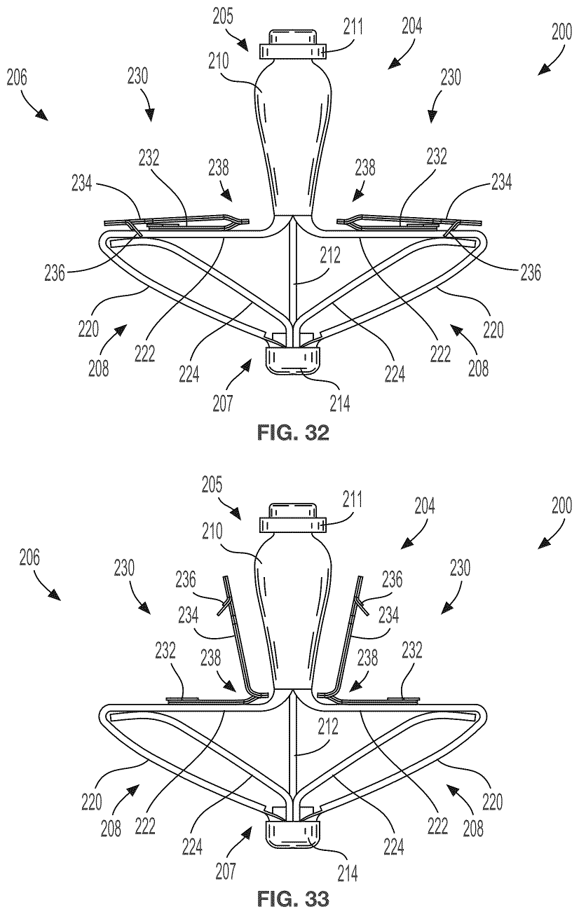

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and movable arms 232, 234.

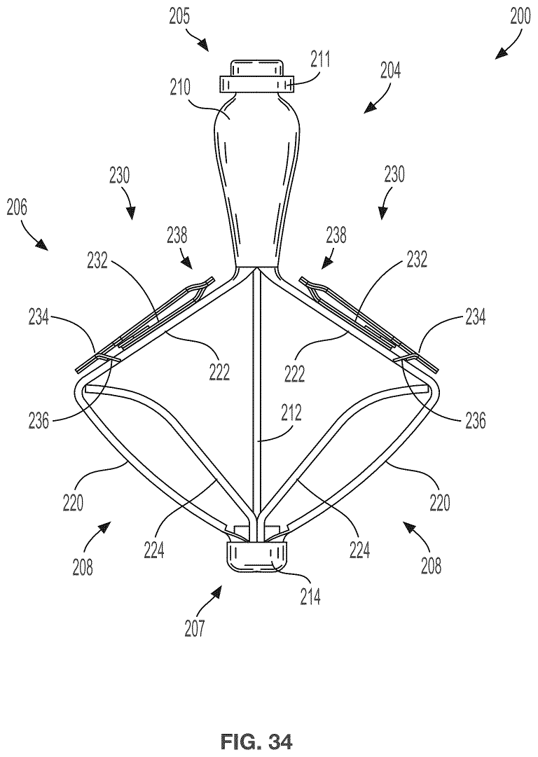

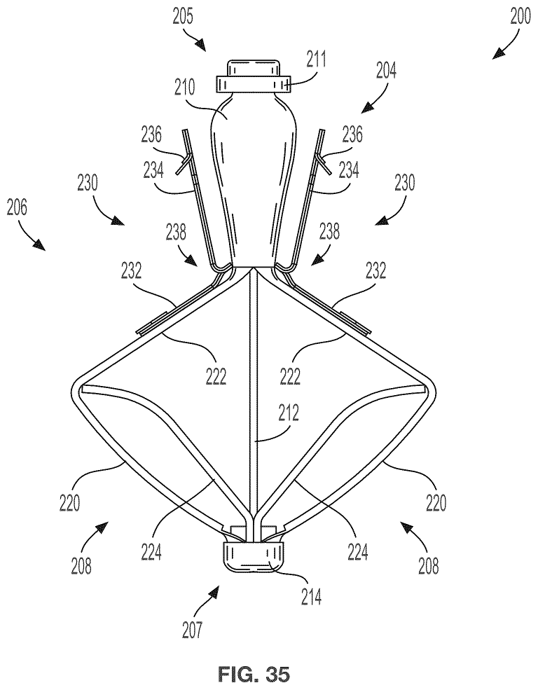

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and movable arms 232, 234.

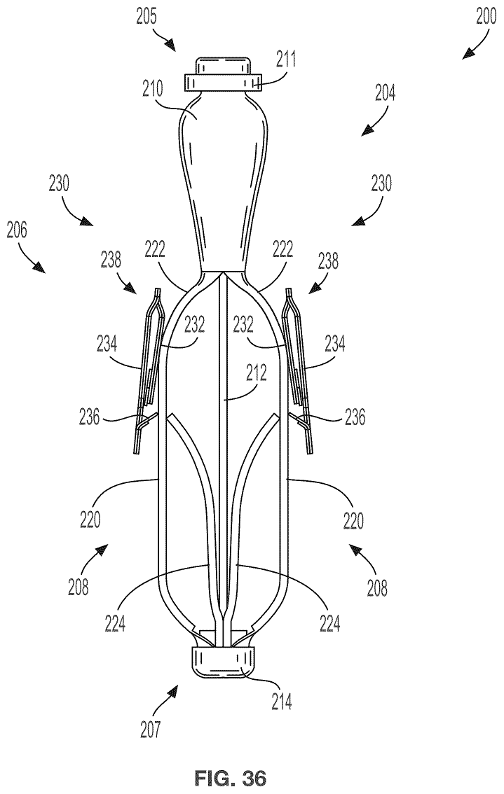

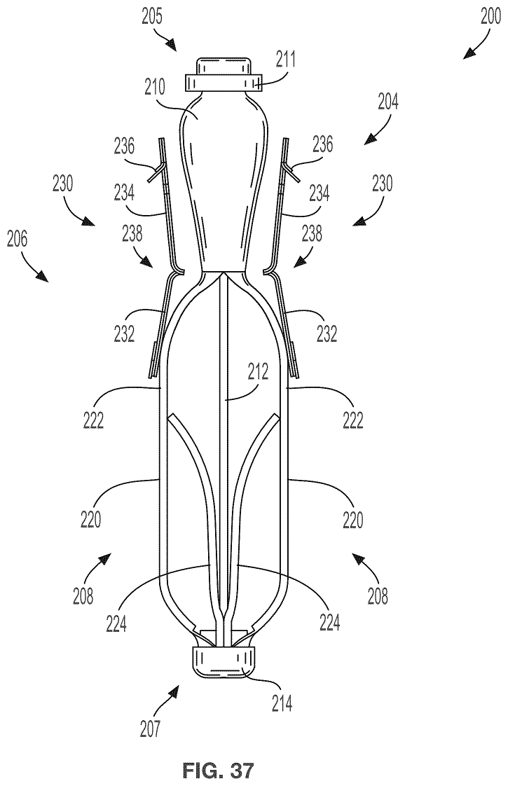

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and movable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent, inhibit, or reduce the likelihood that the device or implant 200 will become entangled in native anatomy (e.g., chordae tendineae CT shown in FIGS. 3 and 4) when positioning and/or retrieving the device or implant 200 into the delivery system 202.

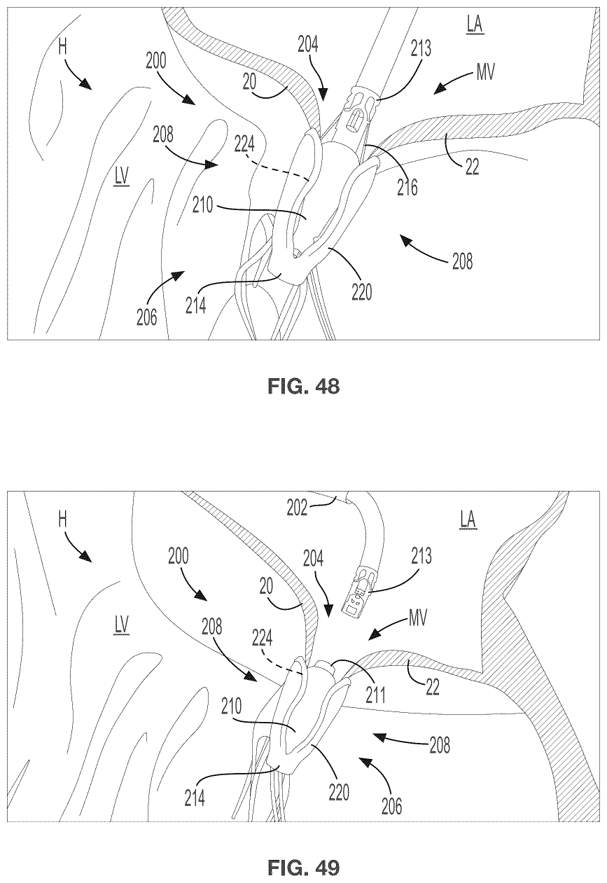

Referring now to FIGS. 38-49, an example device 200 is shown being delivered and deployed within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

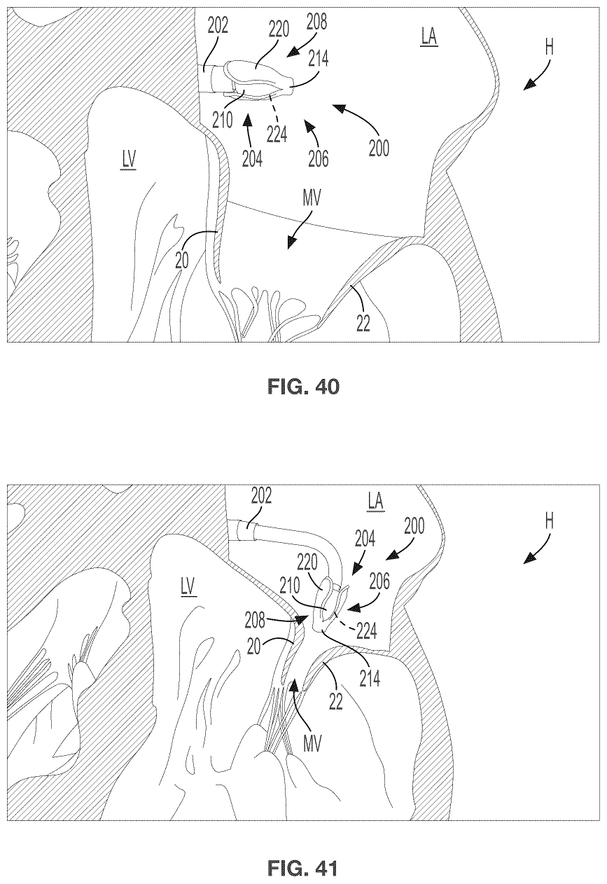

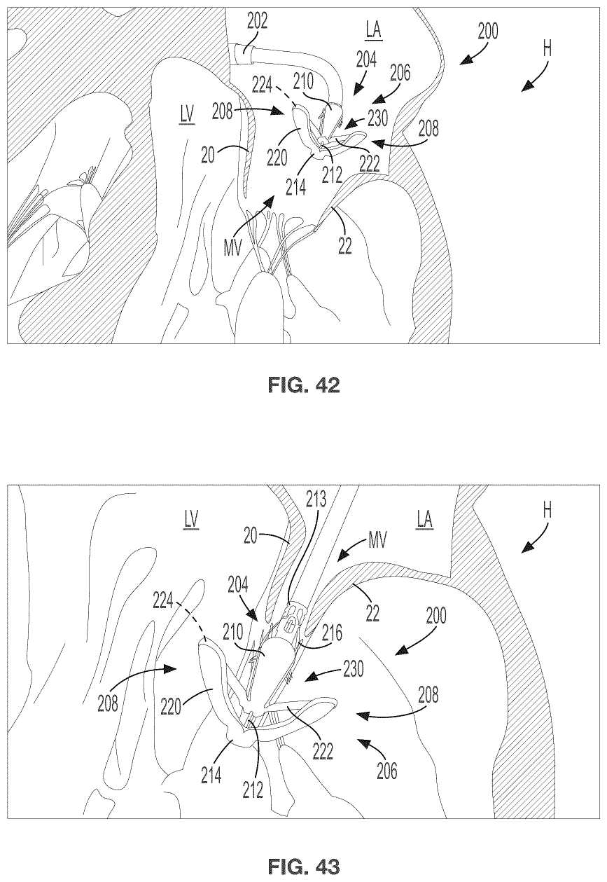

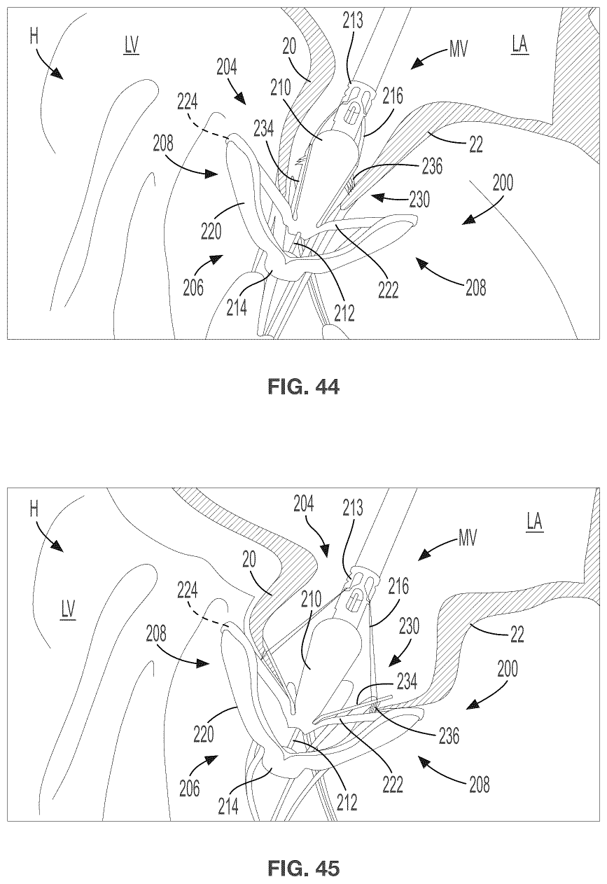

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs, friction-enhancing elements, or securing structures 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy-produced in a wire, sheet, tubing, or laser sintered powder—and are biased to hold the outer paddles 220 closed around the inner paddles 222, coaptation element 210, and/or the clasps 230 pinched around native leaflets 20, 22.

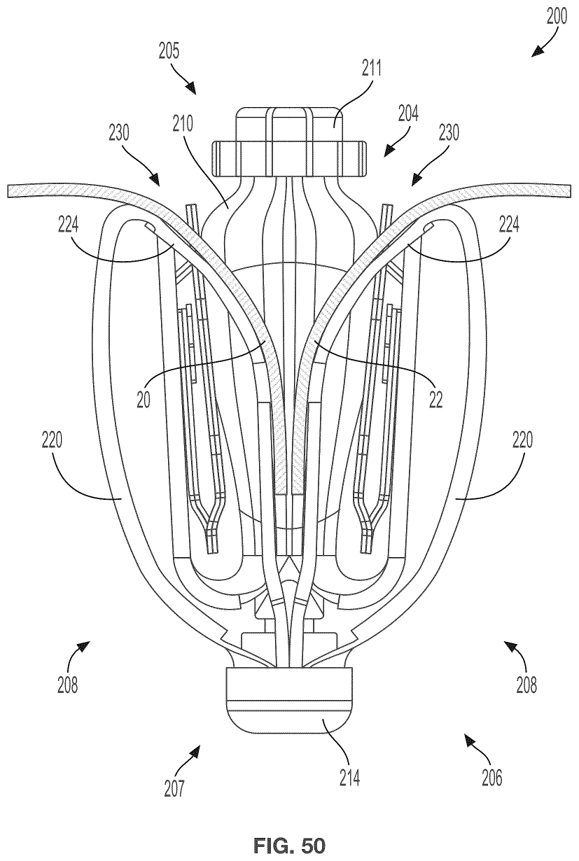

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20, 22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

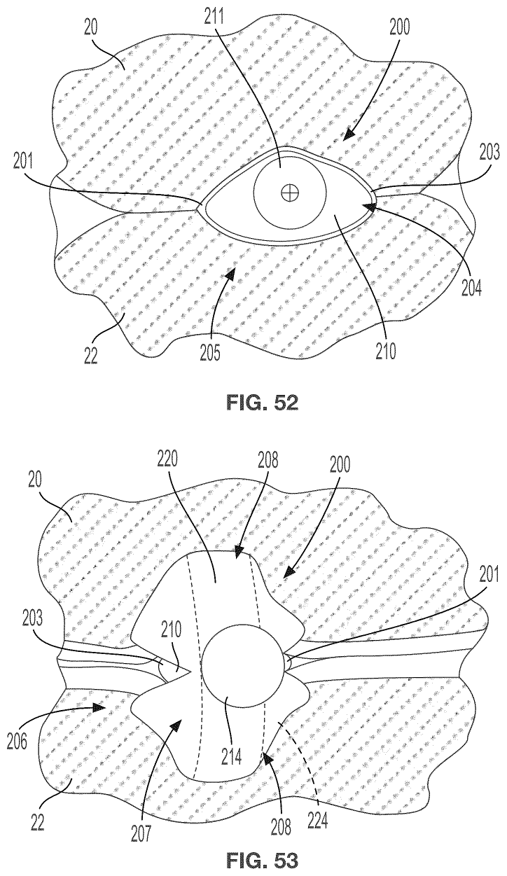

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or inhibited. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial-lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

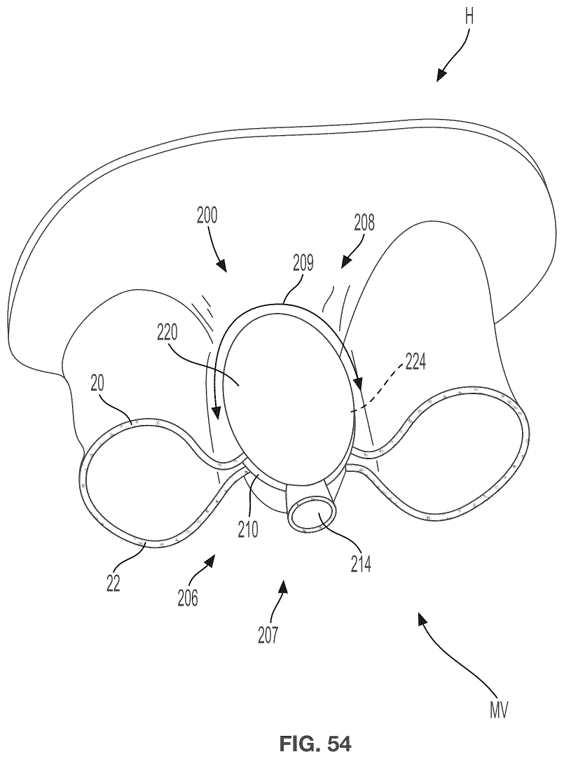

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 209. For example, in FIG. 54, the force on the leaflets 20, 22 by the paddle frames is spread along the entire rounded length of the paddle frame 224, as the leaflets 20 try to open during the diastole cycle.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and U.S. Provisional Patent App. No. 63/217,622, filed on Jul. 1, 2021. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or U.S. Provisional Patent App. No. 63/217,622. Patent Cooperation Treaty International Application No. PCT/US2018/028189 (International Publication No. WO 2018/195215) and U.S. Provisional Patent App. No. 63/217,622 are incorporated herein by reference in their entirety for all purposes.

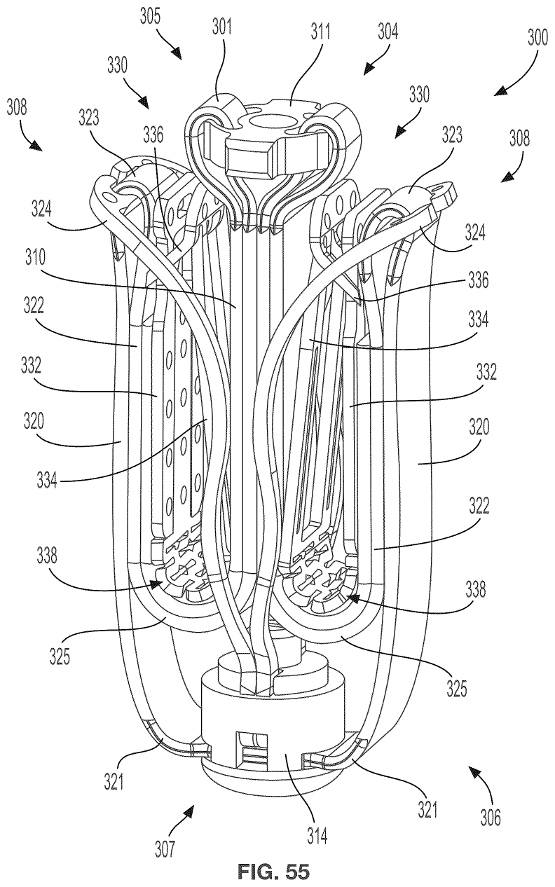

Referring now to FIG. 55, an example of a device or implant 300 (e.g., an implantable prosthetic device, a valve repair device, a valve repair device, etc.) is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable valve repair system (e.g., any valve repair system disclosed in the present application).

The device or implant 300 includes a proximal or attachment portion 305, an anchor portion 306, and a distal portion 307. In some implementations, the device/implant 300 includes a coaptation portion/region 304, and the coaptation portion/region 304 can optionally include a coaptation element 310 (e.g., spacer, plug, membrane, sheet, gap filler, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49 or another capture mechanism described herein or otherwise known) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation member or coaptation element 310, the coaptation member or coaptation element 310 and the anchors 308 can be coupled together in various ways. For example, as shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

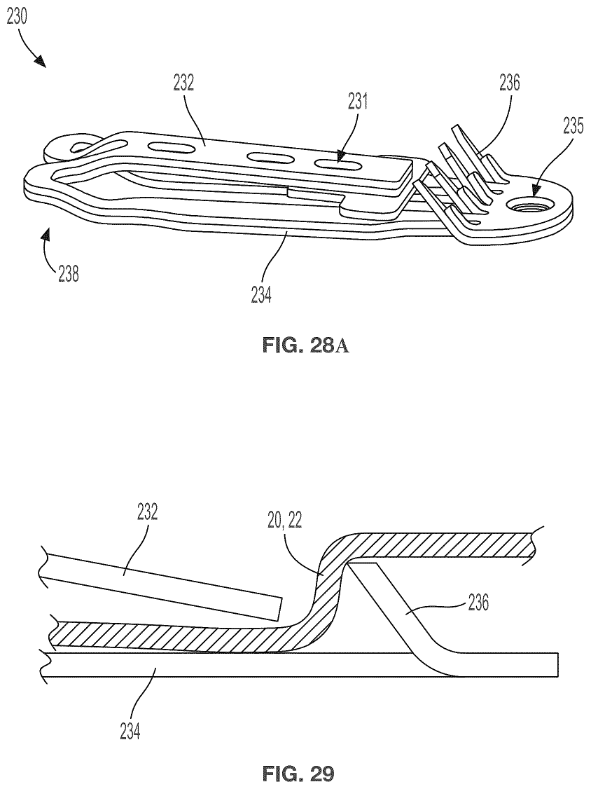

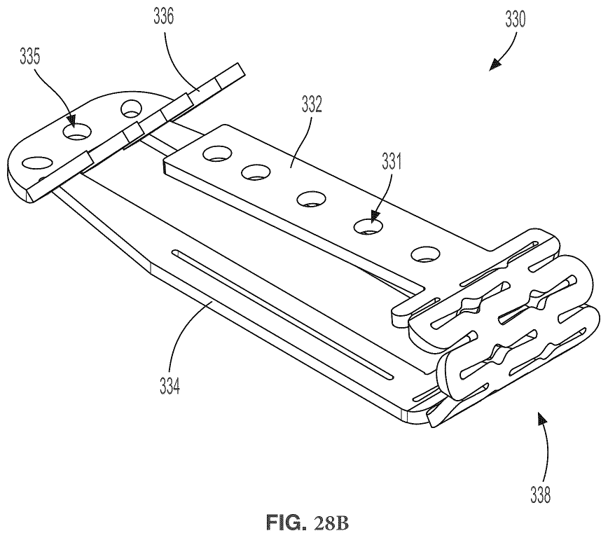

In some implementations, the clasps comprise a movable arm coupled to an anchor. In some implementations, the clasps 330 (as shown in detail in FIG. 28B) include a base or fixed arm 332, a movable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and movable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the movable arms 334 are opened to open the clasps 330 and expose the optional barbs, friction-enhancing elements, or securing structures. The clasps 330 are opened by applying tension to actuation lines (e.g., the actuation lines 216 shown in FIGS. 43-48) attached to holes 335 in the movable arms 334, thereby causing the movable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which can be wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or U.S. Provisional Patent App. No. 63/217,622. Patent Cooperation Treaty International Application No. PCT/US2019/055320 (International Publication No. WO 2020/076898) and U.S. Provisional Patent App. No. 63/217,622 are incorporated herein by reference in their entireties for all purposes. The treatment method(s) described can be performed on a living animal or on a simulation, such as on a cadaver, cadaver heart, simulator (e.g., with the body parts, heart, tissue, etc. being simulated), etc. with the concepts herein mutatis mutandis.

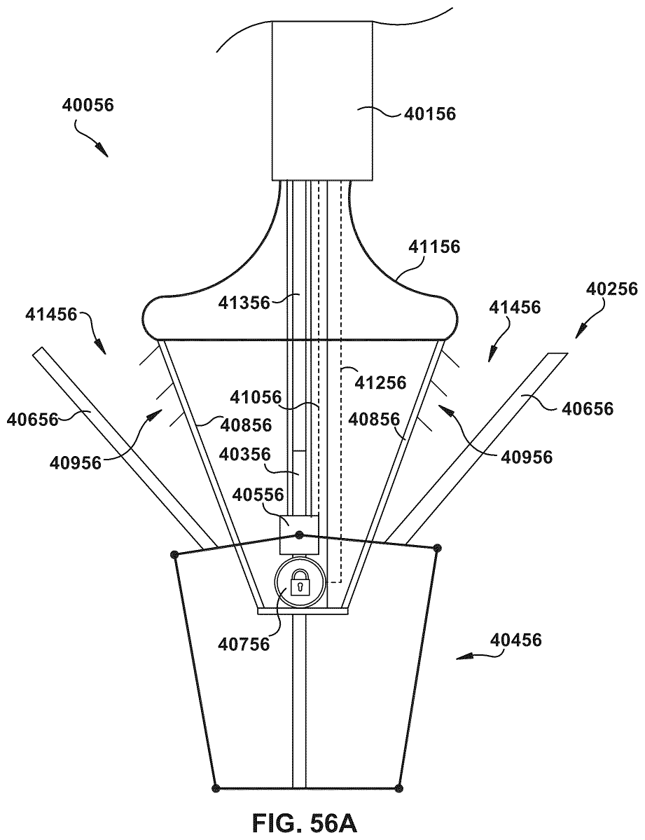

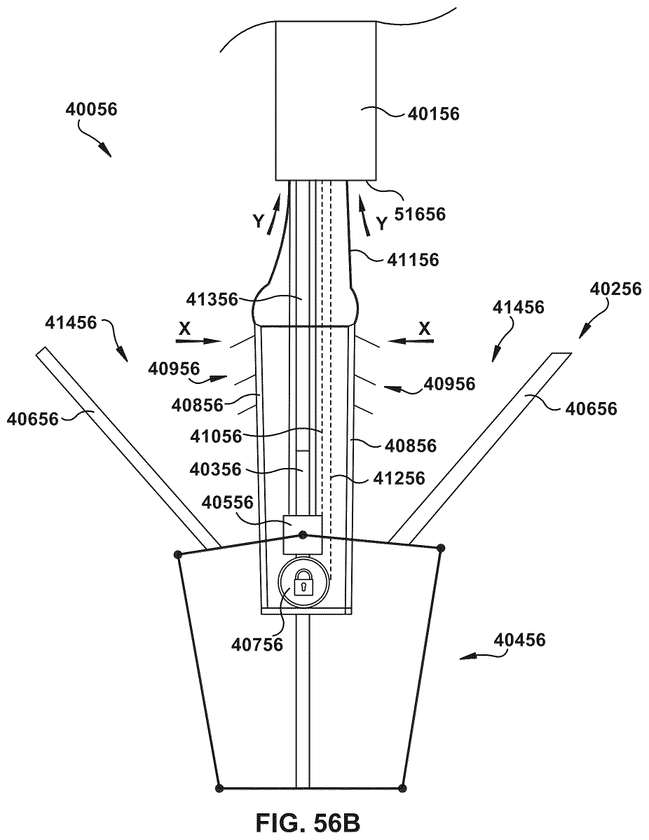

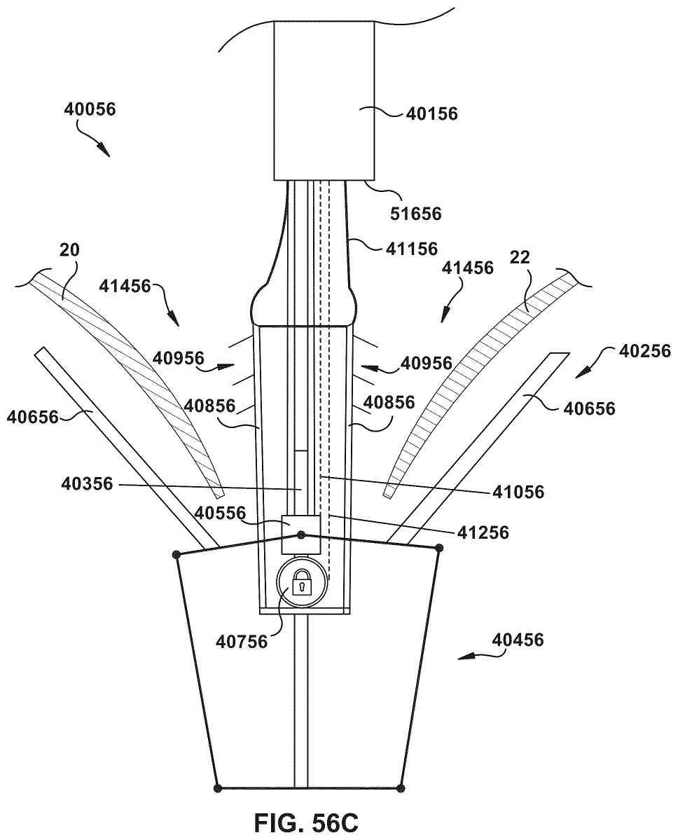

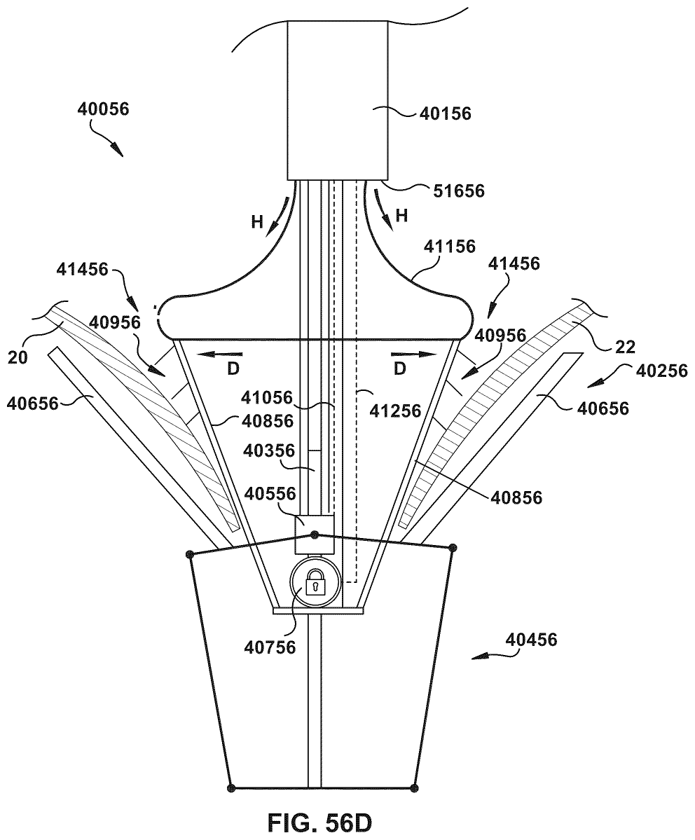

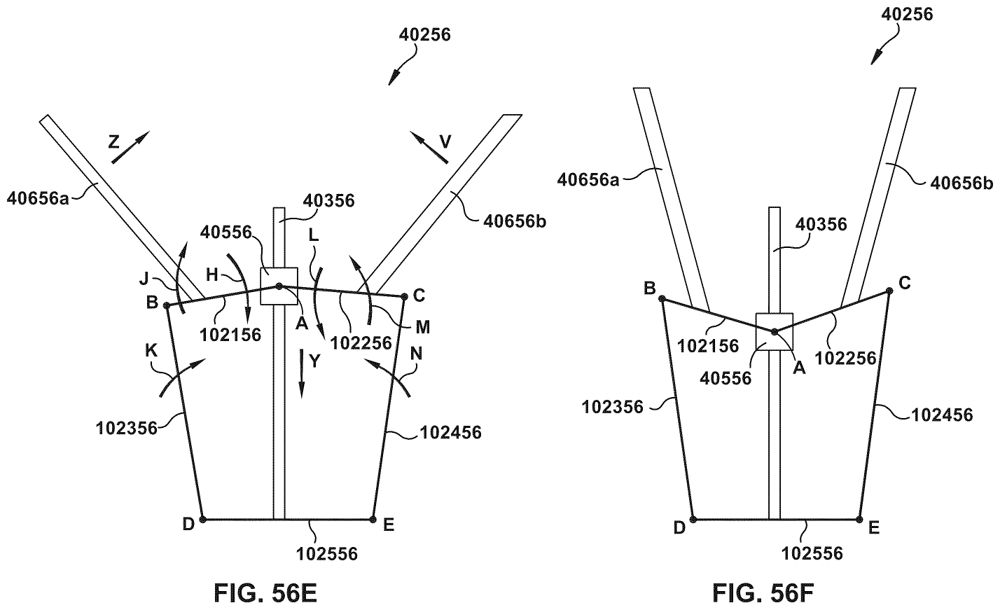

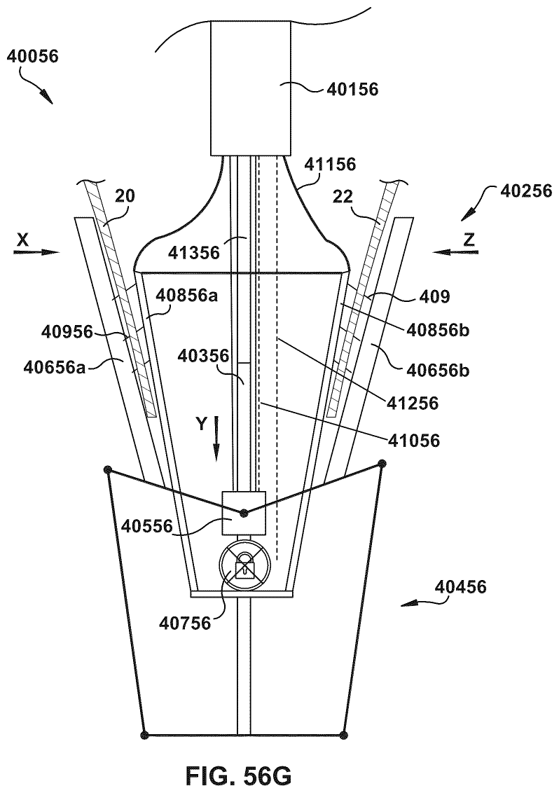

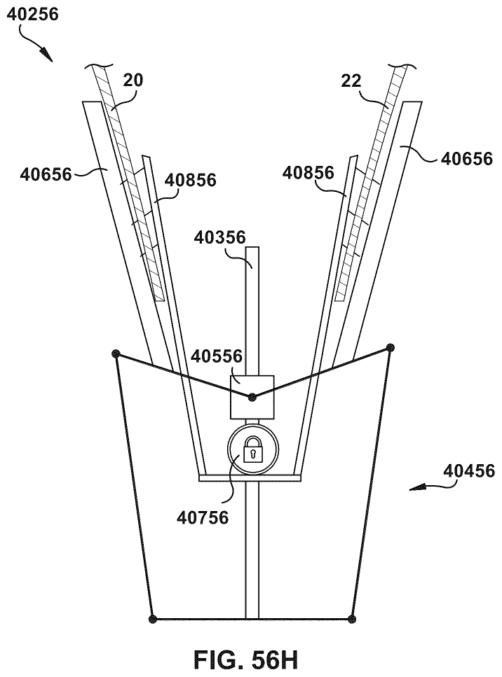

The concepts disclosed by the present application can be used with a wide variety of different valve repair devices. FIGS. 56A-56H illustrate another example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856 (e.g., clasps, arms, pincers, etc.). In some implementations, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include a gripping portion 40956 (e.g., barbs, protrusions, ridges, grooves, textured surfaces, adhesive, etc.) for attaching the gripping members to valve tissue when the valve repair device 40256 is attached to the valve tissue. The gripping member 40856 forms a means for gripping the valve tissue (in particular tissue of the valve leaflets) with a sticking means or portion such as the barbed portion 40956. When the paddles 40656 are in the closed position, the paddles engage the gripping members 40856, such that, when valve tissue is attached to the gripping portion 40956 of the gripping members, the paddles act as holding or securing means to hold the valve tissue at the gripping members and to secure the valve repair device 40256 to the valve tissue. In some implementations, the gripping members 40856 are configured to engage the paddles 40656 such that the gripping portion 40956 engages the valve tissue member and the paddles 40656 to secure the valve repair device 40256 to the valve tissue member. For example, in certain situations, it can be advantageous to have the paddles 40656 maintain an open position and have the gripping members 40856 move outward toward the paddles 40656 to engage valve tissue and the paddles 40656.

While the implementations shown in FIGS. 56A-56H illustrate a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056, a gripper control mechanism 41156, and a lock control mechanism 41256. The paddle control mechanism 41056 is mechanically attached to the coupler 40556 to move the coupler along the shaft, which causes the paddles 40656 to move between the open and closed positions. The paddle control mechanism 41056 can take any suitable form, and can comprise, for example, a shaft, wire tube, hypotube, rod, suture, line, etc. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture, a wire, a rod, a catheter, a tube, a hypotube, etc.

The lock control mechanism 41256 is configured to lock and unlock the lock. The lock 40756 serves as a locking means for locking the coupler 40556 in a stationary position with respect to the shaft 40356 and can take a wide variety of different forms and the type of lock control mechanism 41256 can be dictated by the type of lock used. In some implementations, the lock 40756 includes a pivotable plate having a hole, in which the shaft 40356 of the valve repair device 40256 is disposed within the hole of the pivotable plate. In this implementation, when the pivotable plate is in the tilted position, the pivotable plate engages the shaft 40356 to maintain a position on the shaft 40356, but, when the pivotable plate is in a substantially non-tilted position, the pivotable plate can be moved along the shaft (which allows the coupler 40556 to move along the shaft 40356). In other words, the coupler 40556 is prevented or inhibited from moving in the direction Y (as shown in FIG. 56E) along the shaft 40356 when the pivotable plate of the lock 40756 is in a tilted (or locked) position, and the coupler is allowed to move in the direction Y along the shaft 40356 when the pivotable plate is in a substantially non-tilted (or unlocked) position. In implementations in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position. In some implementations, the pivotable plate of the lock 40756 is biased in the tilted (or locked) position, and the lock control mechanism 41256 is used to move the plate from the tilted position to the substantially non-tilted (or unlocked) position. In some implementations, the pivotable plate of the lock 40756 is biased in the substantially non-tilted (or unlocked) position, and the lock control mechanism 41256 is used to move the plate from the substantially non-tilted position to the tilted (or locked) position.

FIGS. 56E-56F illustrate the valve repair device 40256 moving from an open position (as shown in FIG. 56E) to a closed position (as shown in FIG. 56F). The base assembly 40456 includes a first link 102156 extending from point A to point B, a second link 102256 extending from point A to point C, a third link 102356 extending from point B to point D, a fourth link 102456 extending from point C to point E, and a fifth link 102556 extending from point D to point E. The coupler 40556 is movably attached to the shaft 40356, and the shaft 40356 is fixed to the fifth link 102556. The first link 102156 and the second link 102256 are pivotally attached to the coupler 40556 at point A, such that movement of the coupler 40556 along the shaft 40356 moves the location of point A and, consequently, moves the first link 102156 and the second link 102256. The first link 102156 and the third link 102356 are pivotally attached to each other at point B, and the second link 102256 and the fourth link 102456 are pivotally attached to each other at point C. One paddle 40656a is attached to first link 102156 such that movement of first link 102156 causes the paddle 40656a to move, and the other paddle 40656b is attached to the second link 102256 such that movement of the second link 102256 causes the paddle 40656b to move. In some implementations, the paddles 40656a, 40656b can be connected to links 102356, 102456 or be extensions of links 102356, 102456.

In order to move the valve repair device from the open position (as shown in FIG. 56E) to the closed position (as shown in FIG. 56F), the coupler 40556 is moved along the shaft 40356 in the direction Y, which moves the pivot point A for the first link 102156 and the second link 102256 to a new position. Movement of the coupler 40556 (and pivot point A) in the direction Y causes a portion of the first link 102156 near point A to move in the direction H, and the portion of the first link 102156 near point B to move in the direction J. The paddle 40656a is attached to the first link 102156 such that movement of the coupler 40556 in the direction Y causes the paddle 40656a to move in the direction Z. In addition, the third link 102356 is pivotally attached to the first link 102156 at point B such that movement of the coupler 40556 in the direction Y causes the third link 102356 to move in the direction K. Similarly, movement of the coupler 40556 (and pivot point A) in the direction Y causes a portion of the second link 102256 near point A to move in the direction L, and the portion of the second link 102256 near point C to move in the direction M. The paddle 40656b is attached to the second link 102256 such that movement of the coupler 40556 in the direction Y causes the paddle 40656b to move in the direction V. In addition, the fourth link 102456 is pivotally attached to the second link 102256 at point C such that movement of the coupler 40556 in the direction Y causes the fourth link 102456 to move in the direction N. FIG. 56F illustrates the final position of the valve repair device 40256 after the coupler 40556 is moved as shown in FIG. 56E.

Referring to FIG. 56B, the valve repair device 40256 is shown in the open position (similar to the position shown in FIG. 56E), and the gripper control mechanism 41156 is shown moving the gripping members 40856 to provide a wider gap at the opening 41456 between the gripping members and the paddles 40656. In the illustrated example, the gripper control mechanism 41156 includes a line, such as a suture, a wire, etc. that is threaded through an opening in an end of the gripping members 40856. Both ends of the line extend through the delivery opening 51656 of the delivery device 40156. When the line is pulled through the delivery opening 51656 in the direction Y, the gripping members 40856 move inward in the direction X, which causes the opening 41456 between the gripping members and the paddles 40656 to become wider.

Referring to FIG. 56C, the valve repair device 40256 is shown such that valve tissue 20, 22 is disposed in the opening 41456 between the gripping members 40856 and the paddles 40656. Referring to FIG. 56D, after the valve tissue 20, 22 is disposed between the gripping members 40856 and the paddles 40656, the gripper control mechanism 41156 is used to lessen the width of the opening 41456 between the gripping members and the paddles. That is, in the illustrated example, the line of the gripper control mechanism 41156 is released from or pushed out of the opening 51656 of the delivery member (e.g., tube, shaft, conduit, etc.) in the direction H, which allows the gripping members 40856 to move in the direction D to lessen the width of the opening 41456. While the gripper control mechanism 41156 is shown moving the gripping members 40856 to increase the width of the opening 41456 between the gripping members and the paddles 40656 (FIG. 56C), it should be understood that the gripping members may not need to be moved in order to position valve tissue in the opening 41456. In certain circumstances, however, the opening 41456 between the paddles 40656 and the gripping members 40856 may need to be wider in order to receive the valve tissue.

Referring to FIG. 56G, the valve repair device 40256 is in the closed position and secured to valve tissue 20, 22. The valve repair device 40256 is secured to the valve tissue 20 by the paddles 40656a, 40656b and the gripping members 40856a, 40856b. In particular, the valve tissue 20, 22 is attached to the valve repair device 40256 by the gripping portion 40956 of the gripping members 40856a, 40856b, and the paddles 40656a, 40656b engage the gripping members 40856 to secure the valve repair device 40256 to the valve tissue 20, 22.

In order to move the valve repair device 40256 from the open position to the closed position, the lock 40756 is moved to an unlocked condition (as shown in FIG. 56G) by the lock control mechanism 41256. Once the lock 40756 is in the unlocked condition, the coupler 40556 can be moved along the shaft 40356 by the paddle control mechanism 41056. In the illustrated example, the paddle control mechanism 41056 moves the coupler 40556 in a direction Y along the shaft, which causes one paddle 40656a to move in a direction X and the other paddle 40656b to move in a direction Z. The movement of the paddles 40656a, 40656b in the direction X and the direction Z, causes the paddles to engage the gripping members 40856a, 40856b and secure the valve repair device 40256 to the valve tissue 20, 22.

Referring to FIG. 56H, after the paddles 40656 are moved to the closed position to secure the valve repair device 40256 to the valve tissue 20, 22 (as shown in FIG. 56G), the lock 40756 is moved to the locked condition by the lock control mechanism 41256 (FIG. 56G) to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356 (FIG. 56G). In addition, the valve repair device 40256 is disengaged from the paddle control mechanism 41056 (FIG. 56G), the gripper control mechanism 41156 (FIG. 56G), and the lock control mechanism 41256. Removal of the valve repair device 40256 from the delivery device 40156 allows the valve repair device to remain secured to valve tissue 20, 22 while the delivery device 40156 is removed from a patient.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or U.S. Provisional Patent App. No. 63/217,622. Patent Cooperation Treaty International Application No. PCT/US2019/012707 (International Publication No. WO 2019139904) and U.S. Provisional Patent App. No. 63/217,622 are incorporated herein by reference in their entirety for all purposes.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

During implantation of a device or implant in the native heart valve, movement of the device to the implanted position may be impeded or obstructed by the native heart structures. For example, articulable portions of a device or implant (such as paddle portions of anchors used to secure the device to the native heart valve tissue) may rub against, become temporarily caught, or be temporarily blocked by the chordae tendineae CT (shown in FIGS. 3 and 4) that extend to the valve leaflets. An example device or implant can be configured to reduce the likelihood of the device or implant getting temporarily caught or blocked by the CT. For example, the device or implant can take a wide variety of different configurations that are configured to be actively or passively narrowed to reduce the width of a paddle frame of an anchor portion of the device and, consequently, reduce the surface area of the device, which will make it easier to move the device/implant past and/or through the CT.

The valve repair device 40256 can include any other features of devices discussed elsewhere in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). The treatment method(s) described can be performed on a living animal or on a simulation, such as on a cadaver, cadaver heart, simulator (e.g., with the body parts, heart, tissue, etc. being simulated), etc. with the concepts herein mutatis mutandis.

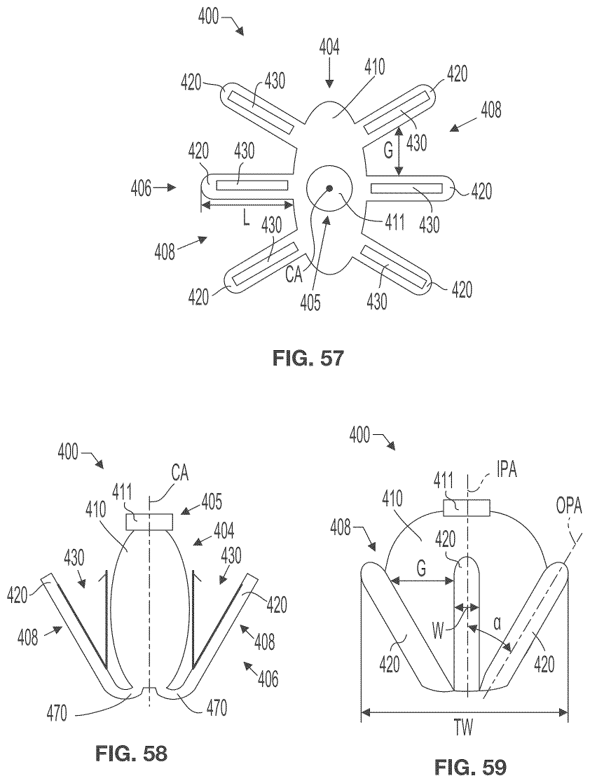

Referring to FIGS. 57-68, various configurations of an example of a device or implant 400 are shown. The device/implant 400 is configured to maneuver more easily into position for implantation in the heart by reducing the contact and/or friction between the native structures of the heart—e.g., chordae—and the device/implant 400. The device/implant 400 can include any other features for a device or implant discussed in the present application or in the applications and patents incorporated by reference herein, and the device 400 can be positioned to engage valve tissue 20, 22 as part of any suitable valve repair system (e.g., any valve repair system disclosed in the present application). In addition, any of the devices or implants described herein can incorporate the features of the device/implant 400.

The device/implant 400 can include a coaptation portion or coaptation portion 404 and an anchor portion 406. The anchor portion can include two or more anchors 408. In some implementations, the coaptation portion 404 optionally includes one or more coaptation elements 410 (e.g., spacers, coaptation elements, gap fillers, etc.). A spacer, coaptation element, coaptation element, etc. 410 can take any suitable form, such as, for example, any form described in the present application.

Each of the anchors 408 include a plurality of paddles 420 (e.g., three in each of the illustrated examples) and one or more clasps 430 (e.g., three in the illustrated example shown in FIGS. 57-59). The clasps 430 can take any suitable form, such as, for example, any form described in the present application.

While the illustrated example shows the anchors 408 each including three paddles 420, it should be understood that the anchors 408 can include any suitable number of paddles 420, such as, for example, two or more paddles, three or more paddles, four or more paddles, five or more paddles, etc.

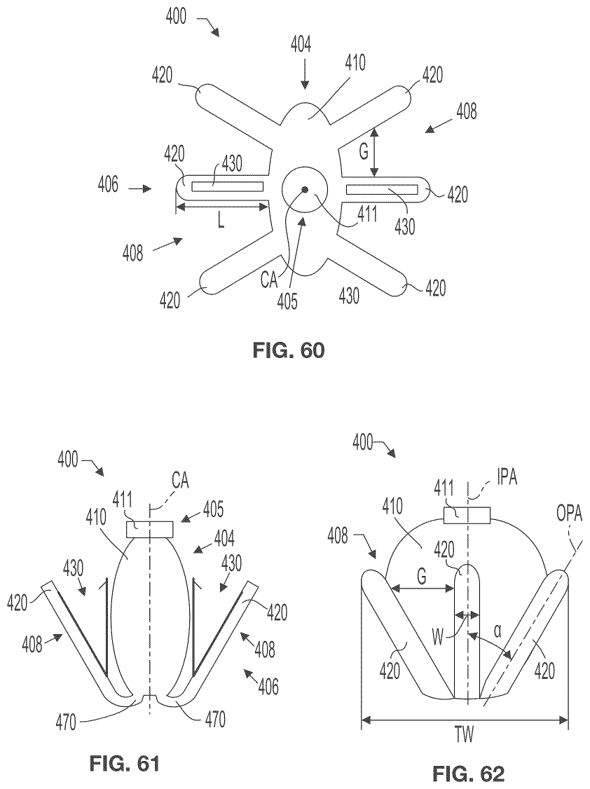

In some implementations, each of the anchors 408 can include a clasp 430 that corresponds to each of the paddles 420 (as shown in FIGS. 57-59), or each anchor 408 can only include a single clasp 430 (e.g., as shown in FIGS. 60-68) that only corresponds to a single paddle of the plurality of paddles 420. It should be understood, however, that each anchor 408 can include any number of paddles 420 that include a corresponding clasp 430 and any number of paddles 420 that do not include a corresponding clasp 430.

The coaptation element 410 and the anchors 408 can be coupled in various ways. For example, as shown in the illustrated examples, the coaptation element 410 and the anchors 408 can optionally be coupled together by integrally forming the coaptation element 410 and the anchors 408 as a single, unitary component. This can be accomplished, for example, by forming the coaptation element 410 and the anchors 408 from a continuous strip of braided or woven material, such as braided or woven nitinol wire. In some implementations, the components are separately formed and are attached together.

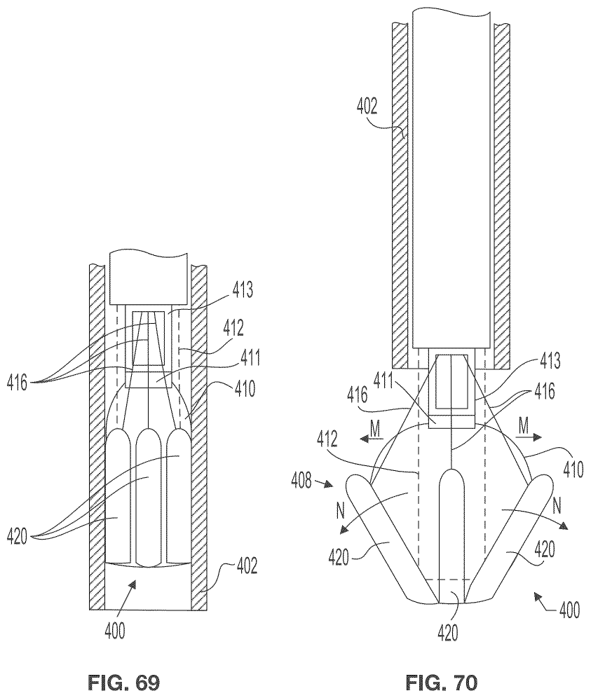

The device or implant 400 can also include an attachment portion 405 for attaching the device 400 to a delivery system 402 (FIGS. 69-73). The delivery system 402 can be the same as or similar to other delivery systems described herein, e.g., 102, 202, and can comprise one or more of a catheter, a sheath, a guide catheter/sheath, a delivery catheter/sheath, a steerable catheter, an implant catheter, a tube, a channel, a pathway, combinations of these, etc. The attachment portion 405 can include a proximal collar 411 for engaging with the delivery system 402 (e.g., with an implant catheter of the delivery system). For example, the proximal collar 411 can be configured to engage with a capture mechanism (e.g., capture mechanism 213 shown in FIGS. 43-49) of the delivery system 402 (e.g., a capture mechanism of an implant catheter).

The anchors 408 are configured to allow the device or implant 400 to more easily maneuver into position for implantation in the heart by reducing the contact and/or friction between the native structures of the heart—e.g., chordae—and the anchors 408. The anchors 408 include a plurality of paddles 420 such that one or more gaps G are formed between the paddles 420. The contact between the native structures of the heart and the anchors 408 is reduced, because the native structures of the heart can extend into the gaps G as the device 400 is moving through the heart. This can allow the device or implant 400 to more easily maneuver within the heart. In addition, the gaps G allow the paddles to flex toward one another during contact with the native structures of the heart—e.g., chordae—and the anchors 408. This flexing can also allow the device/implant 400 to more easily maneuver through the heart. The device/implant can also be configured such that opening or closing the paddles 420 moves the paddles toward one another. This movement of the paddles toward one another can also allow the device/implant 400 to more easily maneuver through the heart.

The anchors 408 can have a total width TW of between 4 mm and 20 mm, such as between 6 mm and 15 mm, such as between 8 mm and 12 mm, such as about 10 mm. Each of the paddles 420 can have a width W of between 0.2 mm and 2 mm, such as between 0.3 and 1.5 mm, such as between 0.5 mm and 1 mm. While each of the paddles 420 is shown as having the same width W, it should be understood that the width W of any of the paddles 420 are not equal to the width W of the other paddles 420. The ratio of the total width TW to the width W can be between 5/1 and 20/1, such as between 7/1 and 15/1, such as about 10/1. The ratio of the total width to the sum of the widths W of the paddles 420 can be between about 2/1 and 15/1, such as between 3/1 and 10/1, such as about 4/1.

In the illustrated example, an inner paddle axis IPA of the inner paddle 420 of the plurality of paddles 420 is substantially aligned with a central axis CA of the device 400, and an outer paddle axis OPA of one or more of the outer paddles 420 extend at an angle α away from the inner paddle axis IPA of the inner paddle 420. The angle α can be between 5 and 60 degrees, such as between 15 and 45 degrees, such as between 20 and 35 degrees.

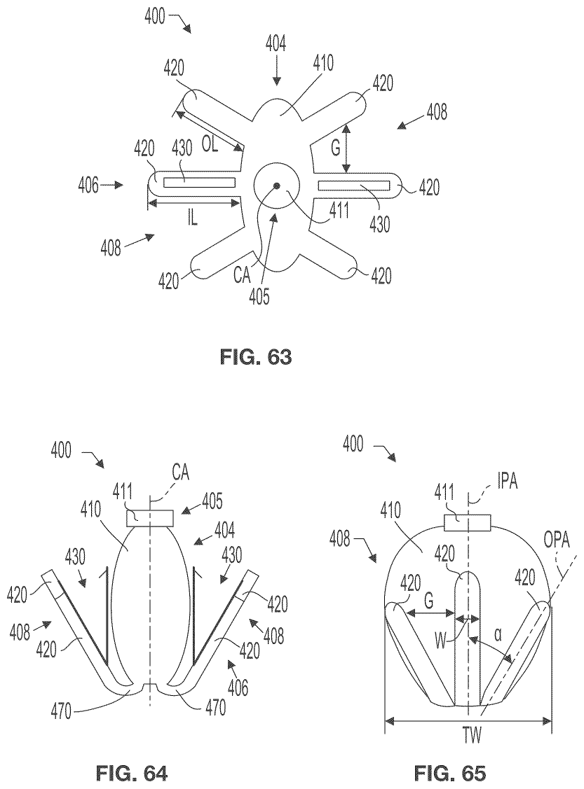

Referring to FIGS. 57-62, each of the paddles 420 has a length L of between 6 mm and 18 mm, such as between 8 mm and 16 mm, such as between 10 mm and 14 mm, such as about 12 mm. While each of the paddles 420 is shown as having the same length L, it should be understood that the length L of any of the paddles 420 not equal to the length L of the other paddles (e.g., see FIGS. 63-68).

FIGS. 60-62 illustrate an example implementation of the device or implant 400 shown in FIGS. 57-59. In this example, the device or implant 400 is identical to the example shown in FIGS. 57-59 except that each anchor 408 only includes a single clasp 430 connected to one paddle of the plurality of paddles 420. In the illustrated example, the clasp 430 is connected to the inner paddle 420 of each anchor 408, and the outer paddles of each anchor 408 do not include a corresponding clasp. In some implementations, each of the outer paddles 420 can include a corresponding clasp 430, and the inner paddle 420 do not include a corresponding clasp. It should be understood that any number of paddles 420 can include a corresponding clasp 430 and any number of paddles 420 do not include a corresponding clasp 430.

FIGS. 63-65 illustrate an example implementation of the device or implant 400 shown in FIGS. 60-62. In this example, the device 400 is identical to the example shown in FIGS. 60-62 except that the inner paddle 420 of each anchor 408 have a length IL that is greater than a length OL of the outer paddles 420. The length IL can be between 6 mm and 18 mm, such as between 8 mm and 16 mm, such as between 10 mm and 14 mm, such as about 12 mm. The length OL can be between 4 mm and 16 mm, such as between 6 mm and 14 mm, such as between 8 mm and 12 mm, such as about 10 mm. A ratio of the length IL to the length OL can be between 10/9 and 2/1, such as between 8/7 and 3/2, such as about 6/5.

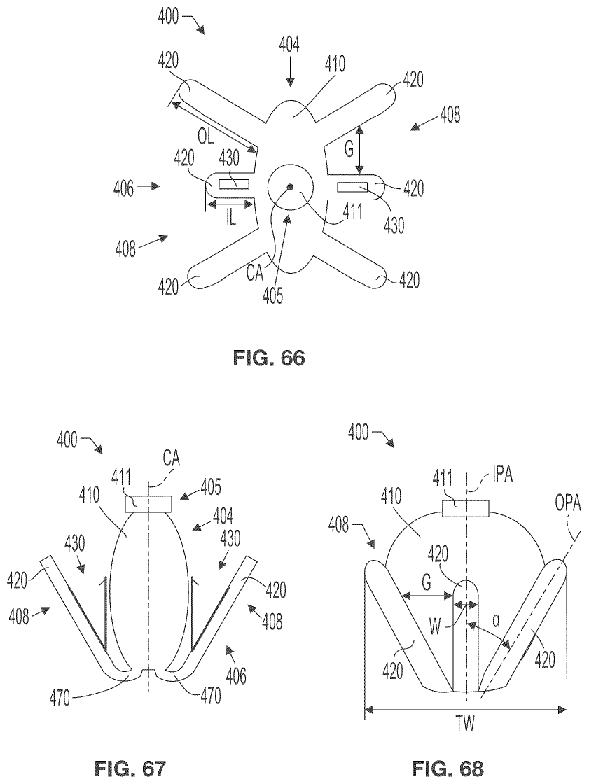

FIGS. 66-68 illustrate an example implementation of the device or implant 400 shown in FIGS. 60-62. In this example, the device 400 is identical to the example shown in FIGS. 60-62 except that the inner paddle 420 of each anchor 408 have a length IL that is less than a length OL of the outer paddles 420. The length OL can be between 6 mm and 18 mm, such as between 8 mm and 16 mm, such as between 10 mm and 14 mm, such as about 12 mm. The length IL can be between 4 mm and 16 mm, such as between 6 mm and 14 mm, such as between 8 mm and 12 mm, such as about 10 mm. A ratio of the length OL to the length IL can be between 10/9 and 2/1, such as between 8/7 and 3/2, such as about 6/5.

While the examples shown in FIGS. 63-68 illustrate each anchor 408 having a single clasp 430 that corresponds to the inner paddle 420, it should be understood that each paddle 420 of the anchors 408 can include a corresponding clasp 430 (e.g., as shown in FIGS. 57-59), or any number of paddles 420 can include a corresponding clasp 430 and any number of paddles 420 do not include a corresponding clasp 430.

Referring to FIGS. 69-73, the device 400 is shown during various stages of deployment from a delivery system 402. The delivery system 402 can take any suitable form, such as, for example, any form described in the present application. While the example of the device/implant 400 illustrated in FIGS. 57-59 is shown with reference to FIGS. 69-73, it should be understood that the deployment of the device/implant 400 from the delivery system 402 also applies to the examples of the device/implant 400 shown in FIGS. 60-68.

Referring to FIG. 69, the device/implant 400 is shown in a compressed position within the delivery system 402. The coaptation element 410 and the paddles 420 are made of a compressible material that allows the device 400 to be in the compressed position as the device 400 is moved into a desired position within the patient's heart. The capture mechanism 413 is connected to the collar 411 of the device/implant 400 while the device/implant 400 is in the delivery system 402 and after deployment of the device/implant 400 from the delivery system 402 until the device/implant 400 is implanted on the native heart valve (e.g., the native mitral valve, tricuspid valve, etc.).

FIG. 70 shows the device or implant 400 in a deployed and closed position. Upon deployment of the device/implant 400 from the delivery system 402, the coaptation element 410 expands in the outward direction M, and the outer paddles 420 of each anchor 408 pivot or articulate outward in the direction N to their normal position such that the gap G (FIGS. 57 and 59) exists between the inner paddle 420 and each of the outer paddles 420.

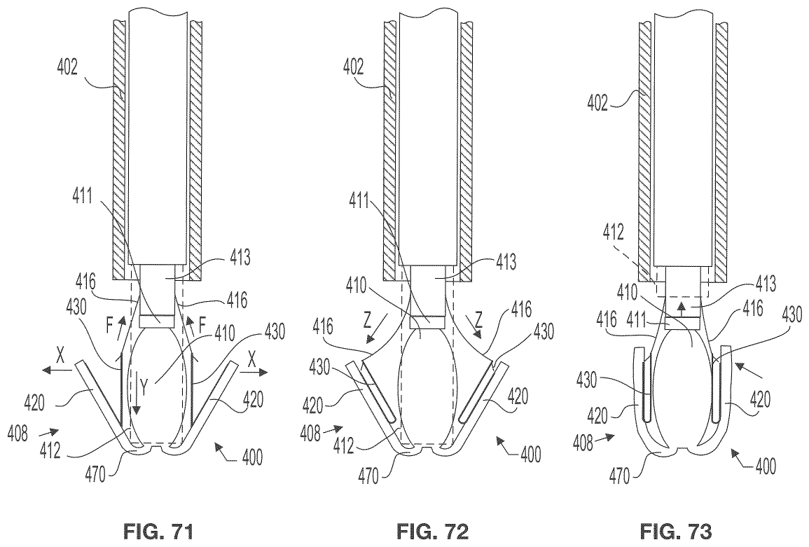

An actuation shaft 412 extends from the delivery system 402 to engage the paddles 420 and move the paddles 420 from the closed position to the open position. Referring to FIG. 71, movement of the actuation shaft 412 in the direction Y to engage and provide a force to the paddles 420 causes the paddles 420 to move in the outward direction X to the open position. That is, the paddles 420 can be pivotally or flexibly connected to the coaptation element 410 at connection point 470 such that the paddles 420 can pivot, flex, and/or articulate outward relative to the coaptation element 410 when a force is provided to the paddles 420. Referring again to FIG. 71, the clasps 430 are maintained in an open position relative to the paddles 420 by a tensioning force F on the clasps 430 by corresponding actuation lines 416 such that a tissue capture area exists between the paddles 420 and the clasps 430.

Referring to FIG. 72, after leaflet tissue is positioned in the tissue capture area between the clasps 430 and the paddles 420, the clasps 430 are moved in the direction Z to capture and secure the device 400 to the tissue. The clasps 430 can be biased in the closed position such that the clasps 430 move to the closed position by releasing the tension force F (FIG. 71) from the actuation lines 416, or the actuation lines 416 can be actively controlled by a user to move the clasps 430 to the closed position.

Referring to FIG. 73, after the device 400 is secured to the leaflet tissue by the paddles 420 and clasps 430, the actuation shaft 412 is disengaged from the paddles 420 and moved back into the delivery system 402 such that the paddles 420 move back to their normally closed positions. After the device 400 is secured to the tissue and the anchors 408 are in the closed position, the capture mechanism 413 is removed from the collar 411 such that the device 400 is no longer attached to the delivery system 402, and the delivery system 402 can be removed from the patient.

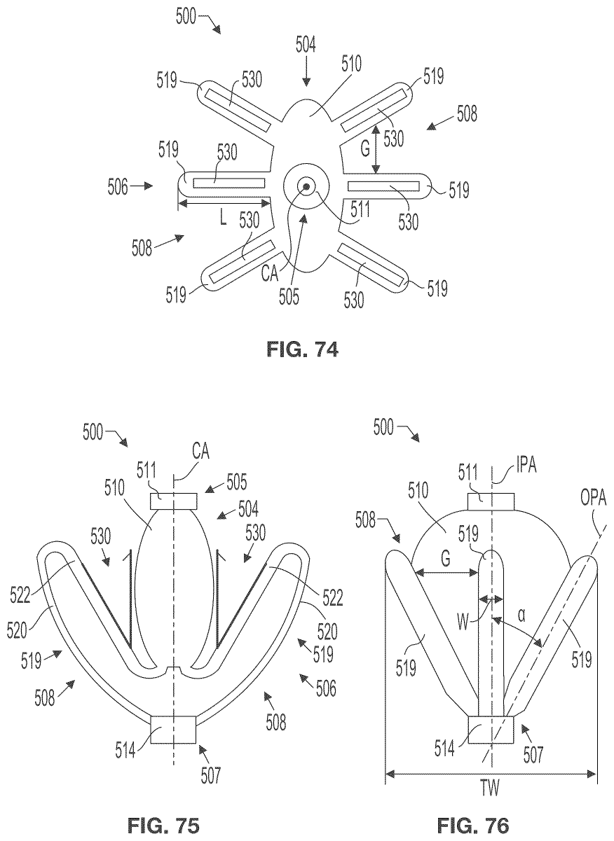

Referring to FIGS. 74-85, various configurations of an example of a device or implant 500 are shown. The device or implant 500 is configured to maneuver more easily into position for implantation in the heart by reducing the contact and/or friction between the native structures of the heart—e.g., chordae—and the device or implant 500. The device or implant 500 can include any other features for a device or implant discussed in the present application or in the applications and patents incorporated by reference herein, and the device 500 can be positioned to engage valve tissue 20, 22 as part of any suitable valve repair system (e.g., any valve repair system disclosed in the present application). In addition, any of the devices/implants described herein can incorporate the features of the device or implant 500.

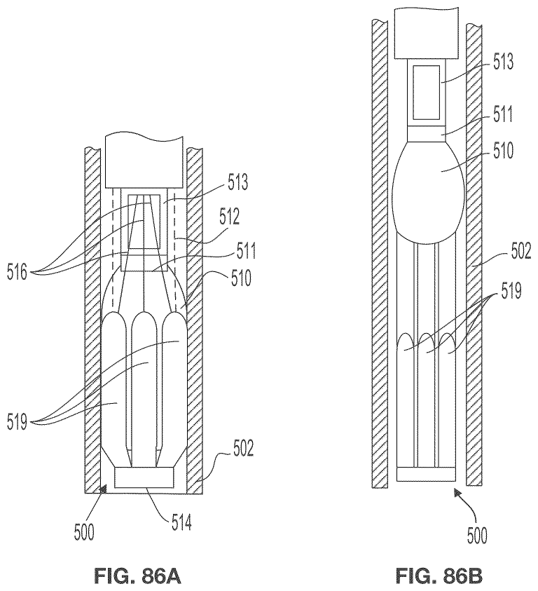

The device or implant 500 includes a coaptation portion 504 (e.g., spacer, coaption element, gap filler, etc.), a proximal or attachment portion 505, an anchor portion 506, and a distal portion 507. In some implementations, the coaptation portion 504 includes a coaptation element 510 (e.g., a spacer, coaption element, gap filler, etc.) that can be used, for example, for implantation between the leaflets 20, 22 of the native mitral valve MV. The coaptation element 510 can take any suitable form, such as, for example, any form described in the present application. The attachment portion 205 includes a first or proximal collar 511 for engaging with a capture mechanism 513 of a delivery sheath or system 202 (See FIGS. 86A, 86B, 87A, 87B, 88, and 89). The proximal collar 511 can take any suitable form, such as, for example, any form described in the present application.

The anchor portion 506 can include two or more anchors 508, where each anchor 508 includes a plurality of paddle members 519 (e.g., three in each of the illustrated examples) and one or more clasps 530 (e.g., three in the illustrated example shown in FIGS. 74-76). The clasps 530 can take any suitable form, such as, for example, any form described in the present application. The distal portion 507 includes a cap 514 that is attached to the paddle portions 519 such that movement of the cap 514 causes the paddle portions 519 to move between open and closed positions. The cap 514 can take any suitable form, such as, for example, any form described in the present application.

The paddle members 519 can each include an outer paddle 520 and an inner paddle 522. The paddle members 519 can be made of, for example, a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body. In some implementations, the paddle members 519 further include a paddle frame (not shown) that supports the inner paddle 522 and the outer paddle 520. The paddle frame can take any suitable form, such as, for example, any form of a paddle frame described in the present application.

The coaptation element 510 is optional. In the illustrated example, the coaptation element 510 and paddle members 519 are formed from a continuous strip of material. The material can be, for example, any of the materials described in the present application for the paddle members 519. In some implementations, the components are separately formed and are attached together. The coaptation element 510 extends from the proximal collar 511 to the inner paddles 522.

The coaptation element 510 has a generally elongated and round shape. In particular, the coaptation element 510 has an elliptical shape or cross-section when viewed from above (e.g., as shown in FIG. 74) and has a tapered shape or cross-section when seen from a front view (as shown in FIG. 75) and a rounded shape or cross-section when seen from a side view (e.g., as shown in FIG. 76). A blend of these three geometries can result in the three-dimensional shape of the illustrated coaptation element 510 that achieves the benefits described herein.

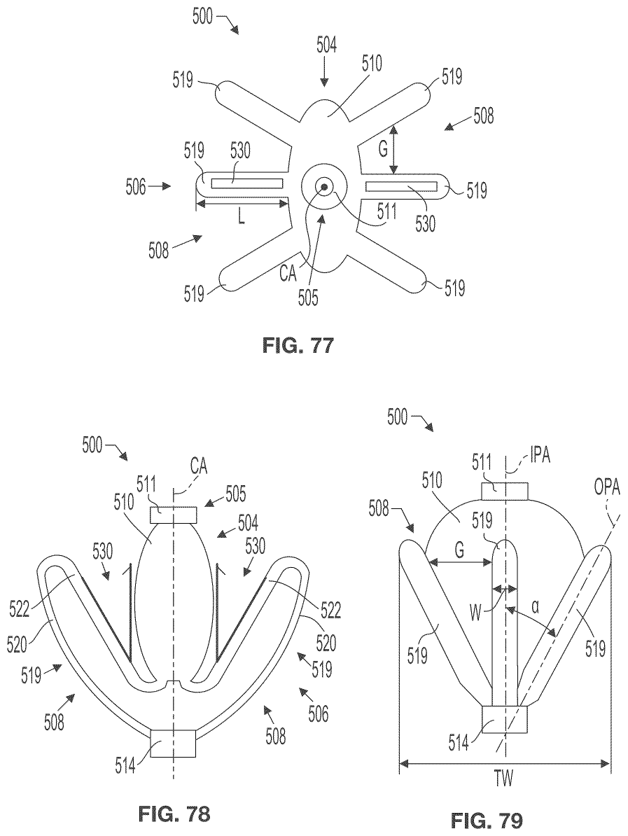

While the illustrated example shows the anchors 508 each including three paddle members 519, it should be understood that the anchors 508 can include any suitable number of paddle members 519, such as, for example, two or more paddle members, three or more paddle members, four or more paddle members, five or more paddle members, etc. In addition, each of the anchors 508 can include clasps 530 that corresponds to each of the paddle members 519 (as shown in FIGS. 74-76), or each anchor 508 can only include a single clasp 530 (e.g., as shown in FIGS. 77-79) that only corresponds to a single paddle member of the plurality of paddle members 519. It should be understood, however, that each anchor 508 can include any number of paddle members 519 that include a corresponding clasp 530 and any number of paddle members 519 that do not include a corresponding clasp 530.

The anchors 508 are configured to allow the device 500 to more easily maneuver into position for implantation in the heart by reducing the contact and/or friction between the native structures of the heart—e.g., chordae—and the anchors 508. The anchors 508 include a plurality of paddles 520 such that one or more gaps G are formed between the paddles 520. The contact between the native structures of the heart and the anchors 508 is reduced, because the native structures of the heart can extend into the gaps G as the device 500 is moving through the heart. This can allow the device 500 to more easily maneuver within the heart. In addition, the gaps G allow the paddles to flex toward one another during contact with the native structures of the heart—e.g., chordae—and the anchors 508. This flexing can also allow the device 500 to more easily maneuver through the heart. The device can also be configured such that opening or closing the paddles 520, 522 moves the paddles toward one another. This movement of the paddles toward one another can also allow the device 500 to more easily maneuver through the heart.

The anchors 508 can have a total width TW of between 4 mm and 20 mm, such as between 6 mm and 15 mm, such as between 8 mm and 12 mm, such as about 10 mm. Each of the paddles 519 can have a width W of between 0.2 mm and 2 mm, such as between 0.3 and 1.5 mm, such as between 0.5 mm and 1 mm. While each of the paddles 519 is shown as having the same width W, it should be understood that the width W of any of the paddles 519 are not equal to the width W of the other paddles 519. The ratio of the total width TW to the width W can be between 5/1 and 20/1, such as between 7/1 and 15/1, such as about 10/1. The ratio of the total width to the sum of the widths W of the paddles 519 can be between about 2/1 and 15/1, such as between 3/1 and 10/1, such as about 4/1.

Referring to FIGS. 74-79, each of the inner paddles 522 has a length L of between 6 mm and 18 mm, such as between 8 mm and 16 mm, such as between 10 mm and 14 mm, such as about 12 mm. While each of the inner paddles 522 is shown as having the same length L, it should be understood that the length L of any of the inner paddles 522 are not equal to the length L of the other inner paddles (e.g., see FIGS. 63-68).

FIGS. 77-79 illustrate an example of the device or implant 500 shown in FIGS. 74-76. In this example, the device 500 is identical to the example shown in FIGS. 74-76 except that each anchor 508 only includes a single clasp 530 attached to one paddle member of the plurality of paddle members 519. In the illustrated example, the clasp 530 is aligned with a middle one of the inner paddle members 522 of each anchor 508, and the outer ones of the inner paddle members 522 do not include a corresponding clasp. In some implementations, each of the outer ones of the paddle members 519 can include a corresponding clasp 530, and the inner ones of the paddle member 519 do not include a corresponding clasp. It should be understood that any number of paddle members 519 can include a corresponding clasp 530 and any number of paddle members 519 do not include a corresponding clasp 530.

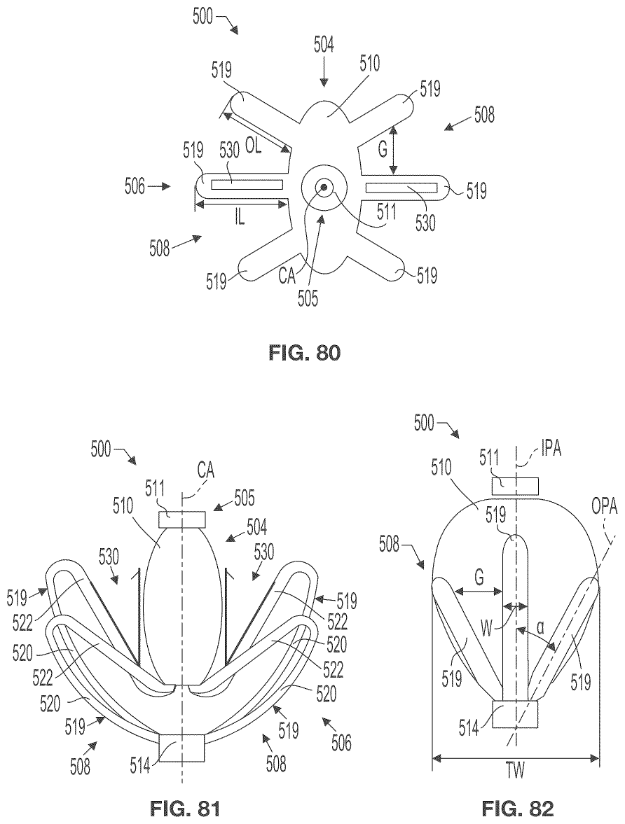

FIGS. 80-82 illustrate an example implementation of the device or implant 500 shown in FIGS. 77-79. In this example, the device/implant 500 is identical to the example shown in FIGS. 77-79 except that the inner ones of the paddles 519 of the anchors 508 have a length IL that is greater than a length OL of the outer ones of the paddles 519. The length IL can be between 6 mm and 18 mm, such as between 8 mm and 16 mm, such as between 10 mm and 14 mm, such as about 12 mm. The length OL can be between 4 mm and 16 mm, such as between 6 mm and 14 mm, such as between 8 mm and 12 mm, such as about 10 mm. A ratio of the length IL to the length OL can be between 10/9 and 2/1, such as between 8/7 and 3/2, such as about 6/5.

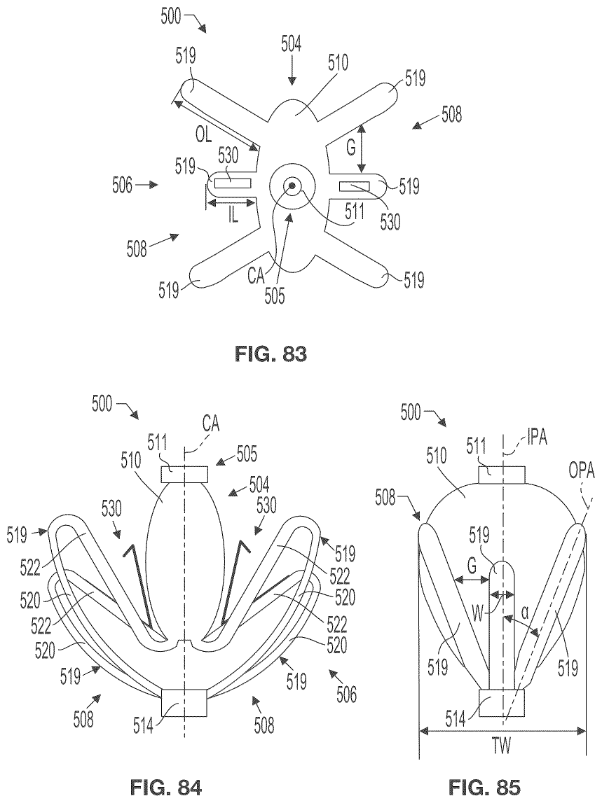

FIGS. 83-85 illustrate an example implementation of the device or implant 500 shown in FIGS. 77-79. In this example, the device 500 is identical to the example shown in FIGS. 77-79 except that the inner paddle member 519 (e.g., strap, link, etc.) of each anchor 508 have a length IL that is less than a length OL of the outer paddle members 519 (e.g., strap, link, etc.). The length OL can be between 6 mm and 18 mm, such as between 8 mm and 16 mm, such as between 10 mm and 14 mm, such as about 12 mm. The length IL can be between 4 mm and 16 mm, such as between 6 mm and 14 mm, such as between 8 mm and 12 mm, such as about 10 mm. A ratio of the length OL to the length IL can be between 10/9 and 2/1, such as between 8/7 and 3/2, such as about 6/5.

While the examples shown in FIGS. 80-85 illustrate each anchor 508 having a single clasp 530 that corresponds to the inner paddle member 519, it should be understood that each paddle member 519 of the anchors 508 can include a corresponding clasp 530 (e.g., as shown in FIGS. 74-76), or any number of paddle members 519 can include a corresponding clasp 530 and any number of paddle members 519 do not include a corresponding clasp 530.

Referring to FIGS. 86A, 87A, and 88-90, the device or implant 500 is shown during various stages of deployment from a delivery system 502. The delivery system 502 can take any suitable form, such as, for example, it can be the same as or similar to other delivery systems herein, e.g., 102, 202, 402, etc., and can comprise one or more of a catheter, a sheath, a guide catheter/sheath, a delivery catheter/sheath, a steerable catheter, an implant catheter, a tube, a channel, a pathway, combinations of these, etc. While the example of the device or implant 500 illustrated in FIGS. 74-76 is shown with reference to FIGS. 86A, 87A, and 88-90, it should be understood that the deployment of the device/implant 500 from the delivery system 502 also applies to the examples of the device/implant 500 shown in FIGS. 77-85.

Referring to FIG. 86A, the device or implant 500 is shown in a compressed position within the delivery system 502. The coaptation element 510 and the paddle members 519 are made of a compressible material that allows the device 500 to be in the compressed position as the device 500 is moved into a desired position within the patient's heart. The capture mechanism 513 is connected to the collar 511 of the device 500 while the device 500 is in the delivery system 502 and after deployment of the device 500 from the delivery system 502 until the device 500 is implanted on the native mitral valve MV (or other native heart valve).

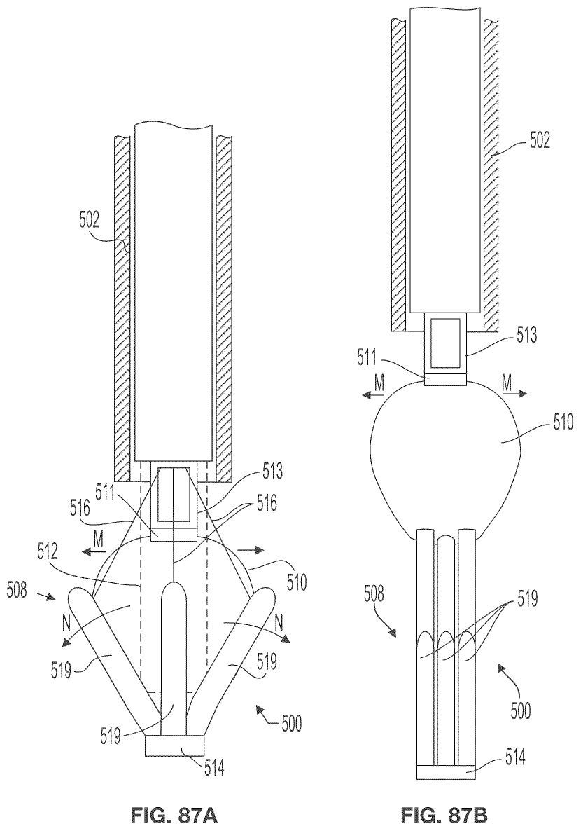

FIG. 87A shows the device 500 in a deployed and closed position. position. Upon deployment of the device 500 from the delivery system 502, the coaptation element 510 expands in the outward direction M, and the outer members 519 of each anchor 508 pivot, outward in the direction N to their normal position such that the gap G (FIGS. 74 and 76) exists between the inner paddle member 519 and each of the outer paddle members 519.

FIG. 86B shows an example similar to the example of FIG. 86A where the paddle members 519 are in an extended position inside of the delivery system 502. This allows the device/implant 500 to be compressed to a smaller size as compared to the example of FIG. 86A, because the paddles are not disposed around the outside of the coaptation element 510. As a result, a smaller delivery system 502 can be used to deliver the same sized device in the example illustrated by FIG. 86B (as compared to the delivery system used in the example illustrated by FIG. 86A).

FIG. 87B shows the device or implant 500 in the FIG. 86B configuration moved out of the delivery system 502. Upon deployment of the device/implant 500 from the delivery system 502, the coaptation element 510 expands in the outward direction M, and the paddle members 519 remain in the extended condition. Once out of the delivery system, the paddle members 519 can be closed (e.g., moved to the positions illustrated by FIG. 87A).

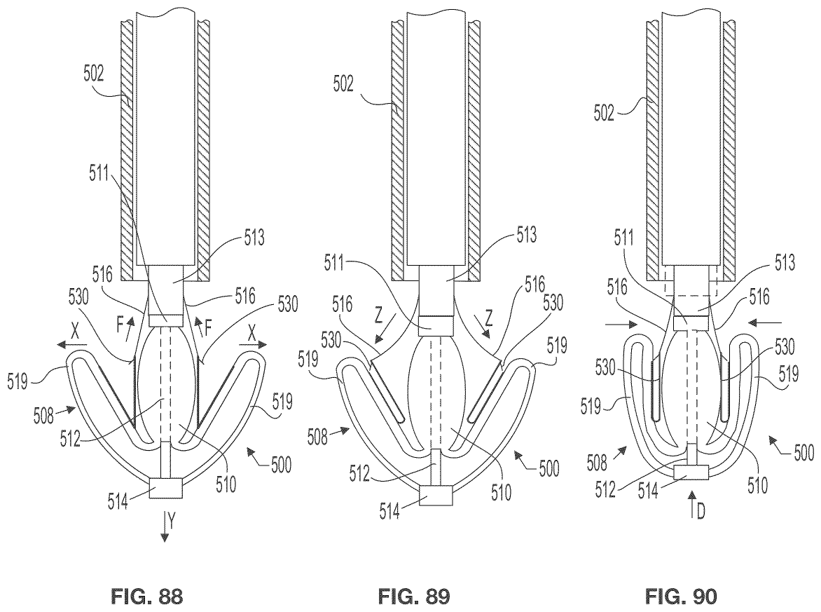

An actuation element 512 (e.g., actuation wire, actuation shaft, etc.) extends from the delivery system 502 to engage the cap 514 and move the paddle members 519 from the closed position to the open position. Referring to FIG. 88, movement of the actuation element 512 to engage the cap 514 to move the cap 514 in the direction Y causes the paddle members 519 to move in the outward direction X to the open position (e.g., similar to the engagement between the actuation element 212 and the cap 214 to move the anchors 208 shown in FIGS. 22-37). The clasps 530 are maintained in an open position relative to the paddle members 519 by a tensioning force F on the clasps 530 by corresponding actuation lines 516 such that a tissue capture area exists between the paddle members 519 and the clasps 530.

Referring to FIG. 89, after leaflet tissue is positioned in the tissue capture area between the clasps 530 and the paddle members 519, the clasps 530 are moved in the direction Z to capture and secure the device/implant 500 to the tissue. The clasps 530 can be biased in the closed position such that the clasps 530 move to the closed position by releasing the tension force F (FIG. 88) from the actuation lines 516, or the actuation lines 516 can be actively controlled by a user to move the clasps 530 to the closed position.

Referring to FIG. 90, after the device or implant 500 is secured to the leaflet tissue by the paddle members 519 and clasps 530, the actuation element 512 moves the cap 514 back to its normal position in the direction D such that the paddle members 519 move to the closed position, and the actuation element 512 is disengaged from the cap 514 and moved back into the delivery system 502. After the device 500 is secured to the tissue and the anchors 508 are in the closed position, the capture mechanism 513 is removed from the collar 511 such that the device 500 is no longer attached to the delivery system 502, and the delivery system 502 can be removed from the patient.

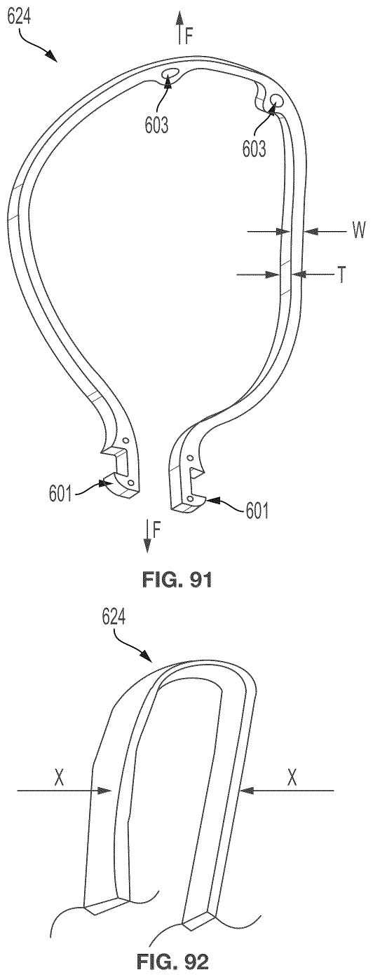

Referring to FIGS. 91-95, an example implementation of a device or implant 600 (FIG. 94) includes an anchor portion 606 having one or more paddle frames 624. The paddle frames 624 are configured to allow the device or implant 600 to maneuver more easily into position for implantation in the heart by reducing the contact and/or friction between the native structures of the heart e.g., chordae—and the device 600. That is, the paddle frames 624 are configured to move between an expanded position (when the device 600 is in a closed position) and a narrowed position (when the device 600 is in an open position), and when the paddle frames 624 are in the narrowed position, the contact between the native structures of the heart and the device 600 is reduced. The device or implant 600 can include any other features for a device or implant discussed in the present application or in the applications and patents incorporated by reference herein, and the device 600 can be positioned to engage valve tissue 20, 22 as part of any suitable valve repair system (e.g., any valve repair system disclosed in the present application). In addition, any of the devices/implants described herein can incorporate the features of the device or implant 600.

Referring to FIG. 94, the device or implant 600 includes a coaptation portion 604, a proximal or attachment portion 605, an anchor portion 606, and a distal portion 607. The coaptation portion 604, attachment portion 605, and distal portion can take any suitable form, such as, for example, the form for these portions of the device 200 shown in FIGS. 22-37, or any other form described in the present application. In some implementations, the coaptation portion 604 optionally includes a coaptation element 610 (e.g., a spacer, coaption element, gap filler, etc.) that can be used, for example, for implantation between the leaflets 20, 22 of the native mitral valve MV. The coaptation element, etc. 610 can take any suitable form, such as, for example, any form described in the present application.

The attachment portion 605 includes a first or proximal collar 611 for engaging with a capture mechanism (e.g., the capture mechanism 213 shown in FIGS. 44-49) of a delivery sheath or system (e.g., the delivery system 202 shown in FIGS. 38-49). The proximal collar 611 can take any suitable form, such as, for example, any form described in the present application.

The distal portion 607 includes a cap 614 that is attached to anchors 608 of the anchor portion 606 such that movement of the cap 614 causes the anchors 608 to move between open and closed positions. The cap 614 can take any suitable form, such as, for example, any form described in the present application. The cap 614 can be moved by extending and retracting an actuation element 612, such as an actuation wire, actuation shaft, etc. (e.g., as described in the present application with respect to device 200 and actuation element 212 shown in FIGS. 22-37).

The anchor portion 606 of the device 600 can take any suitable form, such as, for example, the form of the anchor portion 206 of the device 200 shown in FIGS. 22-37 (except that the paddle frame 224 is replaced with the paddle frame 624 shown in FIGS. 91-95 and described in more detail below), or any other form described in the present application that can incorporate paddle frame 624. The anchor portion 606 can include a plurality of anchors 608, each anchor 608 including outer paddles 620, inner paddles 622, paddle extension members or paddle frames 624, and clasps (e.g., the clasps 230 shown in FIGS. 22-37).

The outer paddles 620 are jointably attached to the inner paddles 622 by connection portions 623 and to the cap 614 of the distal portion 607, and the inner paddles 622 are jointably attached to the coaptation element 610. In this manner, the anchors 608 are configured similar to legs in that the inner paddles 622 are like upper portions of the legs, the outer paddles 620 are like lower portions of the legs, and the connection portions 623 are like knee portions of the legs.

The paddle frames 624 have first connection members 601 (mating surfaces, connectors, cutouts, fasteners, etc.) (FIGS. 91 and 95) for attaching the paddle frames 624 to the cap 614 at the distal portion 607 such that the paddle frames 624 are fixedly connected to the cap 614. The connection members 601 can be, for example, cutouts that mate with corresponding cutouts in the cap. The paddle frames 624 have one or more second connection members 603 (FIGS. 91 and 95) that connect to the connection portions 623 between the inner and outer paddles 622, 620 such that the paddle frames 624 are fixedly connected to the anchors 608. The connection members 603 can be, for example, eyelets that allow the paddle frames 624 to be sewn to a cover that is also sewn to the inner and outer paddles 622, 620. In some implementations, the paddle frames 624 are formed of a material that is more rigid and stiff than the material forming the paddles 622, 620 so that the paddle frames 624 provide support for the paddles 622, 620.

The paddle frames 624 provide additional pinching force between the inner paddles 622 and the coaptation element 610. The paddle frames assist in wrapping the leaflets around the sides of the coaptation element 610 for a better seal between the coaptation element 610 and the leaflets. That is, the paddle frames 624 can be configured with a round three-dimensional shape extending from the cap 614 to the connection portions 623 of the anchors 608. The connections between the paddle frames 624, the outer and inner paddles 620, 622, the cap 614, and the coaptation element 610 can constrain the movement of each of these parts (e.g., to the movements and positions described with reference to FIGS. 22-37). In particular the connection portion 623 is constrained by its connection between the outer and inner paddles 620, 622 and by its connection to the paddle frame 624. Similarly, the paddle frame 624 is constrained by its attachment to the connection portion 623 (and thus the inner and outer paddles 622, 620) and to the cap 614.

Configuring the paddle frames 624 in this manner provides increased surface area compared to the inner paddles 622 alone. This can, for example, make it easier to grasp and secure the native leaflets. The increased surface area can also distribute the clamping force of the paddles 620 and paddle frames 624 against the native leaflets over a relatively larger surface of the native leaflets in order to further protect the native leaflet tissue. In some implementations, the increased surface area of the paddle frames 624 can also allow the native leaflets to be clamped to the device or implant 200, such that the native leaflets coapt entirely around the coaptation element 610. This can, for example, improve sealing of the native leaflets and thus prevent, inhibit, or further reduce valvular regurgitation.

The paddle frames 624 are configured to move between an expanded position (e.g., as shown in FIG. 91) and a narrowed position (e.g., as shown in FIGS. 92 and 95). When in the expanded position, the paddle frames 624 have the increased surface area that provides the above-mentioned advantages for securing the device 600 to a native valve of the heart. When in the narrowed position, the paddle frames 624 have a reduced width relative to the paddle frames in the expanded position, which allows the device 600 to more easily maneuver into position for implantation in the heart by reducing the contact and/or friction between the native structures of the heart—e.g., chordae—and the device 600. Movement of the anchors 608 between the open and closed positions cause the paddle frames 624 to move between the expanded and narrowed positions.

In the illustrated example, an actuation element 612 (e.g., an actuation wire, actuation shaft, etc.) extends from a delivery system (e.g., any delivery system described in the present application) and engages the cap 614 to move the cap 614 in the directions Y relative to the coaptation element or spacer 610 to enable actuations of the device 600. The actuation element 612 can engage and move the cap by any suitable means, such as, for example, any means provided in the present application. Movement of the cap 614 away from the coaptation element 610 causes the anchors 608 to move to the opened position (as shown in FIG. 94), and movement of the coaptation element 610 toward the coaptation element 610 causes the anchors to move to the closed position.

The configuration of the paddle frames 624 and the connections of the paddle frames 624 with the cap 614 and the connection portions 623 of the anchors 608 causes the paddle frames 624 to be in the expanded position when the anchors 608 are in the closed position and in the narrowed position when the anchors 608 are in the open position. That is, referring to FIG. 91, movement of the anchors 608 to the open position causes a tension force F on the paddle frames 624 because the cap 614 is moving away from the coaptation element 610 in the direction Y (FIG. 94) and the paddle frames 624 are fixedly connected to the cap 614 and the connection portions 623 of the anchors 608.

Referring to FIGS. 91 and 92, the paddle frames 624 have a width W and a thickness T that is greater than the width W. The thickness T being greater than the width W increases the extent to which the paddle frame 624 compresses in the direction X when the tension force F is applied to the paddle frame 624. This is because the stiffness of the paddle frame in the direction of the width W is less than the stiffness in the direction of the thickness T. In some implementations, a ratio of the thickness T to the width W is between 10/9 and 3/1, such as between 5/4 and 2/1, such as between 4/3 and 3/2.

Referring to FIG. 95, the paddle frame 624 have a length L2 and a total width W2 when in the narrowed position. The Length L2 can be between 9 mm and 21 mm, such between 12 mm and 18 mm, such as about 15 mm. The width W2 can be between 3 mm and 12 mm, such as between 5 mm and 10 mm, such as between 7 mm and 9 mm, such as about 8 mm. A ratio of a total width (not shown) of the paddle frame 624 in the expanded position to the total width W2 can be is between 10/9 and 3/1, such as between 5/4 and 2/1, such as between 4/3 and 3/2. A ratio of a length (not shown) of the paddle frame 624 in the expanded position to the length L2 of the paddle frame 624 can be between 10/9 and 3/1, such as between 5/4 and 2/1, such as between 4/3 and 3/2.

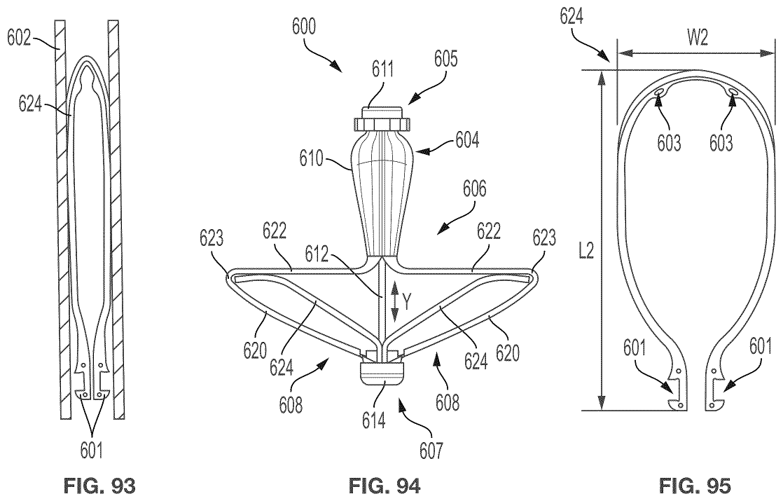

Referring to FIG. 93, the paddle frame 624 is shown in a compressed position within the delivery system 602. The delivery system 602 can take any suitable form, such as, for example, it can be the same as or similar to other delivery systems herein, e.g., 102, 202, 402, 502, etc., and can comprise one or more of a catheter, a sheath, a guide catheter/sheath, a delivery catheter/sheath, a steerable catheter, an implant catheter, a tube, a channel, a pathway, combinations of these, etc. . . . The configuration of the paddle frame 624 allows the paddle frame to more easily maintain the compressed position within the delivery system 602. That is, the paddle frame 624 having a thickness T (FIG. 91) that is greater than its width W (FIG. 91) allows the paddle frame 624 to more easily compress because the stiffness of the paddle frame in the direction of the width W is less than the stiffness in the direction of the thickness T.

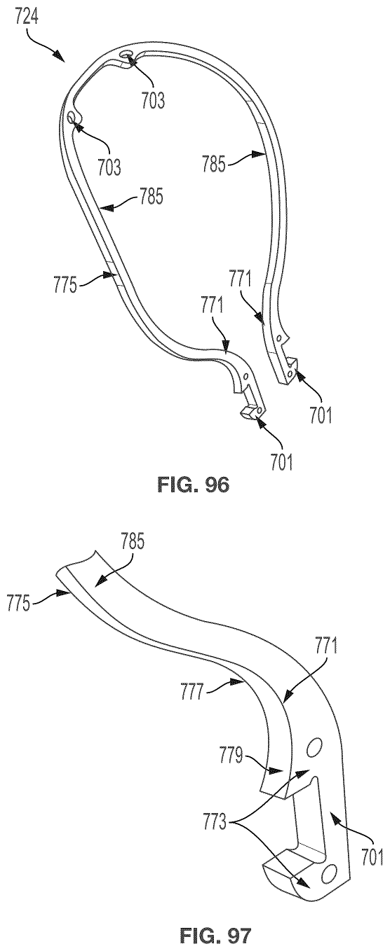

Referring to FIGS. 96-98, 101, and 104, an example of a paddle frame 724 for a device or implant (e.g., device 200 shown in FIGS. 22-37, device 600 shown in FIG. 94, or any other suitable device) includes a main support section 785, first connection members 701 (e.g., cutouts, complementary surfaces, connectors, fasteners, etc.) for attaching to a cap of the device or implant, second connection members 703 for attaching to anchors of the device, and a transition portion 771 that extends between the first connection members 701 and the main support section 785. The paddle frame 724 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

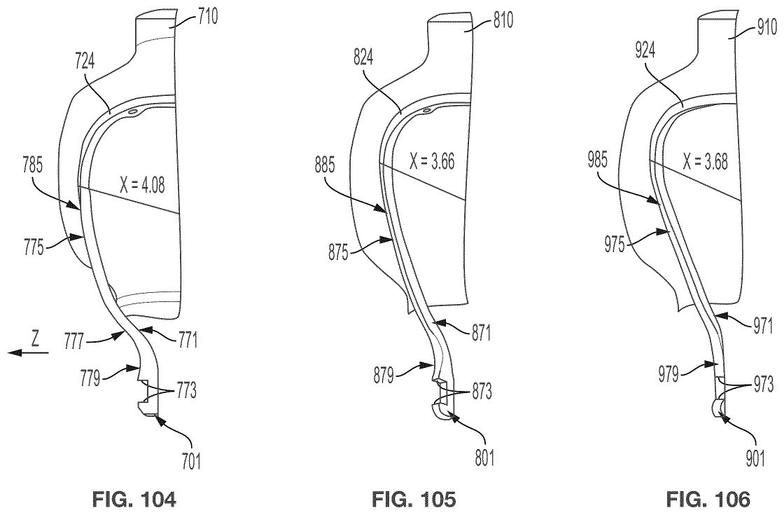

The connection members 701 of the paddle frame 724 include extension portions 773 that are configured to extend into the cap of the device or implant to connect the paddle frame 724 to the cap. In this example, an outer surface 775 of the main support section 785, an outer surface 777 of the transition portion 771, and an outer surface 779 of the connection member 701 are substantially aligned such that each of these outer surfaces are facing the same direction Z (FIG. 104).

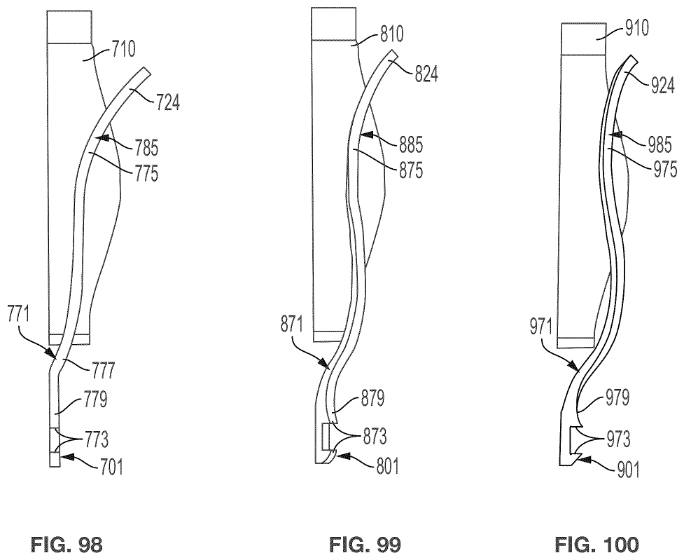

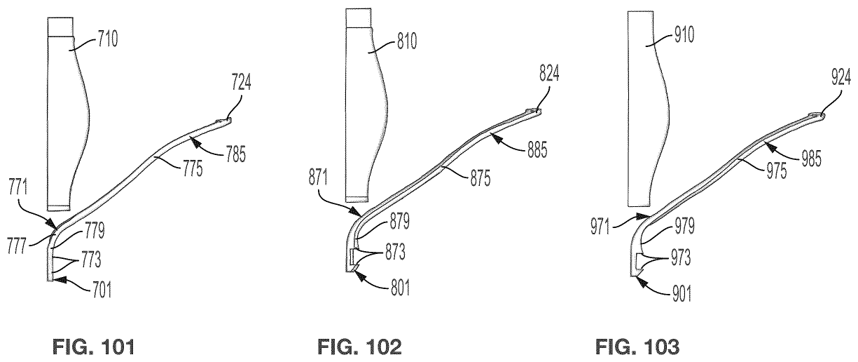

Referring to FIG. 98, the paddle frame 724 is shown in a closed position relative to a coaptation element or spacer 710 of a device or implant. Referring to FIGS. 101 and 104, the paddle frame is shown in an open position relative to the coaptation element 710. The coaptation element 710 can take any suitable form, such as, for example any form described in the present application.

Referring to FIGS. 99 and 102 and 105, an example of a paddle frame 824 for a device or implant (e.g., device 200 shown in FIGS. 22-37, device 600 shown in FIG. 94, or any other suitable device) includes a main support section 885, first connection members 801 (e.g., cutouts, complementary surfaces, connectors, fasteners, etc.) for attaching to a cap of the device or implant, second connection members (e.g., connection members 603 shown in FIG. 91) for attaching to anchors of the device, and a transition portion 871 that extends between the first connection members 801 and the main support section 885. The paddle frame 824 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The connection members 801 of the paddle frame 824 include extension portions 873 that are configured to extend into the cap of the device or implant to connect the paddle frame 824 to the cap. In this example, an outer surface 879 of the connection member 801 is disposed at an angle of about 45 degrees from an outer surface 875 of the main support section 885 such that the transition portion 871 is twisted about its axis.

The paddle frame can be shape set with the twist illustrated by FIGS. 99 and 102. In some implementations, the paddle frame can be shape set with the shape shown in FIGS. 96, 98, and 101, the connection members 801 can be twisted to the position illustrated by FIGS. 99 and 102, and held in the twisted orientation by the attachment to the cap. In some implementations, paddle frame 824 can be shape set with the connection members 801 set in the position illustrated by FIGS. 99 and 102, but twisted back to the position illustrated by FIGS. 96, 98, and 101 by the connection to the cap.

Referring to FIG. 99, the paddle frame 724 is shown in a closed position relative to a coaptation element or spacer 810 of a device or implant. Referring to FIGS. 102 and 105, the paddle frame is shown in an open position relative to the coaptation element 810. The coaptation element 810 can take any suitable form, such as, for example any form described in the present application.

The angle between the outer surface 879 of the connection member 801 and the outer surface 875 of the main support section 885 (and the corresponding twisted transition portion 871) results in an increased torque and resulting stress in the material of the paddle frame 824 as the paddle frames are moved from the closed position to the open position. This increased torque and resulting stress in the material of the paddle frame is due to the paddle frames being fixedly connected to both the inner and outer paddles (at the transition between the two) and the cap of the device or implant. When the cap pulls on the outer paddles, the twist of the transition portion 871 is spread along the length of the paddle frame. As a result, the paddle frame 824 narrows more than a paddle frame that does not include a twisted translation portion 871 when the cap pulls the paddles to the open position. This additional reduction of the width of the paddle frame allows the device or implant to more easily maneuver into position for implantation in the heart by reducing the contact and/or friction between the native structures of the heart—e.g., chordae—and the device.

While the illustrated example, shows the outer surface 879 being disposed at an angle of about 45 degrees from the outer surface 875, it should be understood that the outer surface 879 can be disposed at any other suitable angle relative to the outer surface 875 such that the paddle frame torques and moves to a more narrowed position (as compared to a paddle frame that does not have a twisted translation portion) when the paddle frame moves from a closed position to an opened position.

In general, a greater amount of twist results in more resulting torque, stress, and paddle narrowing. For example, in FIGS. 100, 103 and 106, an example of a paddle frame 924 with a twist of 90 degrees. In the example illustrated by FIGS. 100, 103 and 106, the device or implant (e.g., device 200 shown in FIGS. 22-37, device 600 shown in FIG. 94, or any other suitable device) includes a main support section 985, first connection members 901 (e.g., cutouts, complementary surfaces, connectors, fasteners, etc.) for attaching to a cap of the device or implant, second connection members (e.g., connection members 603 shown in FIG. 91) for attaching to anchors of the device, and a transition portion 971 that extends between the first connection members 901 and the main support section 985. The paddle frame 924 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The connection members 901 of the paddle frame 924 include extension portions 973 that are configured to extend into the cap of the device or implant to connect the paddle frame 924 to the cap. In this example, an outer surface 979 of the connection member 901 is disposed at an angle of about 90 degrees from an outer surface 975 of the main support section 985 such that the transition portion 971 is twisted about its axis.

The paddle frame can be shape set with the twist illustrated by FIGS. 100 and 103. In some implementations, the paddle frame can be shape set with the shape shown in FIGS. 96, 98, and 101, the connection members 901 can be twisted to the position illustrated by FIGS. 100 and 103, and held in the twisted orientation by the attachment to the cap. In some implementations, paddle frame 924 can be shape set with the connection members 901 set in the position illustrated by FIGS. 100 and 103, but twisted back to the position illustrated by FIGS. 96, 98, and 101 by the connection to the cap.

Referring to FIG. 100, the paddle frame 924 is shown in a closed position relative to a coaptation element or spacer 910 of a device or implant. Referring to FIGS. 103 and 106, the paddle frame is shown in an open position relative to the coaptation element 910. The coaptation element 910 can take any suitable form, such as, for example any form described in the present application.

The angle between the outer surface 979 of the connection member 901 and the outer surface 975 of the main support section 985 (and the corresponding twisted transition portion 971) results in an increased torque and resulting stress in the material of the paddle frame 924 as the paddle frames are moved from the closed position to the open position. This increased torque and resulting stress in the material of the paddle frame is due to the paddle frames being fixedly connected to both the inner and outer paddles (at the transition between the two) and the cap of the device or implant. When the cap pulls on the outer paddles, the twist of the transition portion 971 is spread along the length of the paddle frame. As a result, the paddle frame 924 narrows more than a paddle frame that does not include a twisted translation portion 971 when the cap pulls the paddles to the open position. This additional reduction of the width of the paddle frame allows the device or implant to more easily maneuver into position for implantation in the heart by reducing the contact and/or friction between the native structures of the heart—e.g., chordae—and the device.

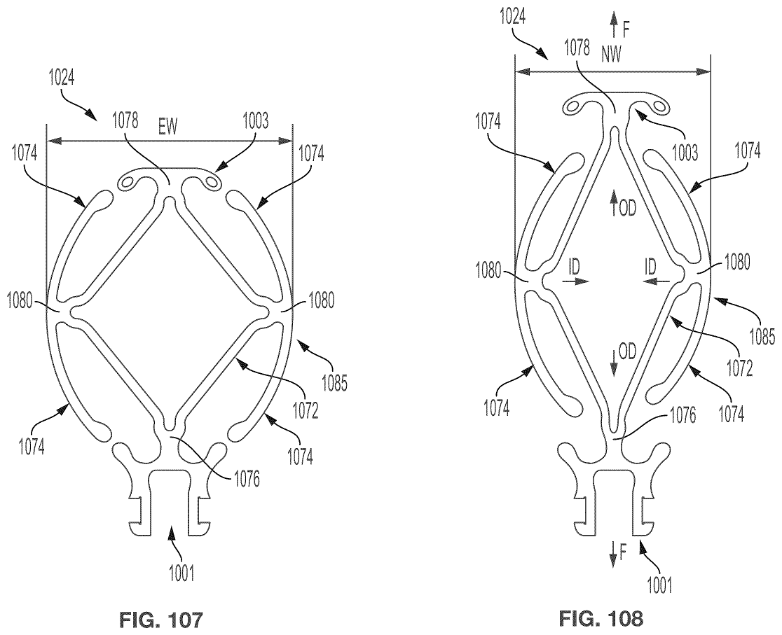

Referring to FIGS. 107 and 108, an example of a paddle frame 1024 for a device or implant (e.g., device 200 shown in FIGS. 22-37, device 600 shown in FIG. 94, or any other suitable device) includes a main support section 1085, first connection members 1001 (e.g., cutouts, complementary surfaces, connectors, fasteners, etc.) for attaching to a cap of the device or implant, and second connection members 1003 for attaching to anchors of the device. The paddle frame 1024 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1085 includes an inner frame portion 1072 and an outer frame portion 1074. The inner frame portion 1072 is connected to the connection members 1001, 1003 at connection points 1076, 1078, respectively. The inner frame portion 1072 is configured to cause the paddle frame 1024 to move from a normal, expanded position (FIG. 107) when the anchors of the device or implant are in a closed position to a narrowed position (FIG. 108) when the anchors of the device move to an open position. The outer frame portion 1074 is connected to the inner frame portion at connection points 1080, and the outer frame portion 1074 defines the total width (e.g., the expanded width EW shown in FIG. 107 and the narrowed width NW shown in FIG. 108) of the paddle frame 1024.

In the illustrated example, the inner frame portion 1072 of the main support section 1085 is a diamond shape. Referring to FIG. 108, when the anchors of the device or implant move to the open position, the paddle frames 1024 experience a tension force F because the paddle frames 1024 are fixedly connected to the cap and the transition portion between the inner and outer paddles of the device. This tension force F on the paddle frames 1024 cause the connection points 1076, 1078 to move in an outward direction OD, which causes the connection points 1080 to move in an inward direction ID. The movement of the connection points 1080 in the inward direction ID cause the outer frame portion 1074 to move in the inward direction ID such that the total width of the paddle frame 1024 moves from the expanded width EW (FIG. 107) to the narrowed width NW (FIG. 108). The movement of the paddle frame 1024 to the narrowed position allows the device or implant to more easily maneuver into position for implantation in the heart by reducing the contact and/or friction between the native structures of the heart—e.g., chordae—and the device.

The expanded width EW of the paddle frame 1024 can be between 5 mm and 15 mm, such as between 7 mm and 12 mm, such as between 9 mm and 11 mm, such as about 10 mm. The narrowed width NW of the paddle frame 1024 can be between 3 mm and 12 mm, such as between 5 mm and 10 mm, such as between 7 mm and 9 mm, such as about 8 mm. A ratio of the expanded width EW to the narrowed width NW can be between 10/9 and 3/1, such as between 5/4 and 2/1, such as between 4/3 and 3/2.

While the illustrated example, shows inner frame portion 1072 of the main support section 1085 being a diamond shape, it should be understood that the inner frame portion 1072 can take any form that allows the paddle frame 1024 to move to the narrowed position when the tension force F is applied to the paddle frame 1024 such that the paddle frame can allow the device or implant to more easily maneuver into position for implantation in the heart.

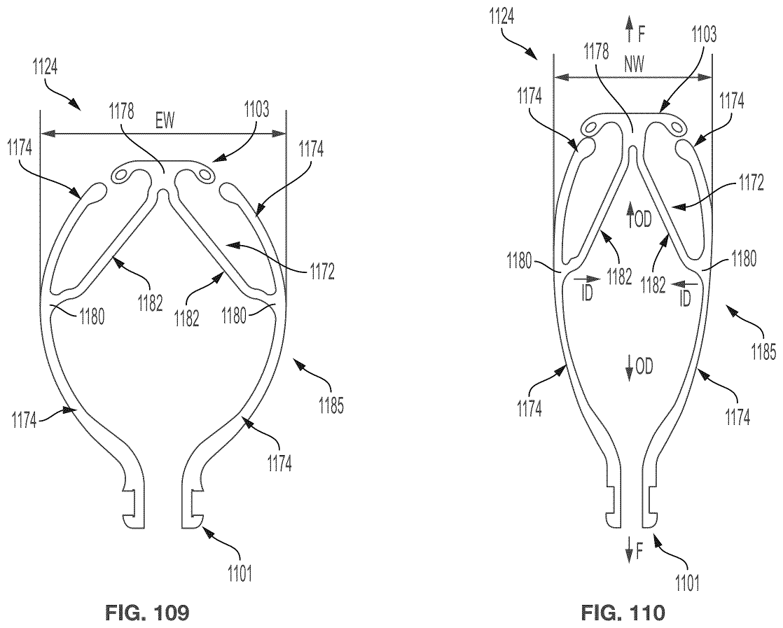

Referring to FIGS. 109 and 110, an example of a paddle frame 1124 for a device or implant (e.g., device 200 shown in FIGS. 22-37, device 600 shown in FIG. 94, or any other suitable device) includes a main support section 1185, first connection members 1101 (e.g., cutouts, complementary surfaces, connectors, fasteners, etc.) for attaching to a cap of the device or implant, and second connection members 1103 for attaching to anchors of the device. The paddle frame 1124 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1185 includes an inner frame portion 1172 and an outer frame portion 1174. The inner frame portion 1172 is connected to the connection members 1103 at connection point 1178. The outer frame portion 1174 is connected to the inner frame portion 1172 at connection points 1180, and the outer frame portion 1174 extends to the connection member 1101. The outer frame portion 1174 defines the total width (e.g., the expanded width EW shown in FIG. 109 and the narrowed width NW shown in FIG. 110) of the paddle frame 1124. The inner frame portion 1172 is configured to cause the paddle frame 1124 to move from a normal, expanded position (FIG. 109) when the anchors of the device or implant are in a closed position to a narrowed position (FIG. 110) when the anchors of the device move to an open position.

In the illustrated example, the inner frame portion 1172 of the main support section 1185 includes arms 1182 that extend inward from the connection points 1180 and meet at the connection point 1178 such that the inner frame portion 1172 has a triangular shape. Referring to FIG. 110, when the anchors of the device or implant move to the open position, the paddle frames 1124 experience a tension force F because the paddle frames 1124 are fixedly connected to the cap and the transition between the inner and outer paddles of the device. This tension force F on the paddle frames 1124 cause the connection point 1178 and the connection member 1101 to move in an outward direction OD, which causes the connection points 1080 to move in an inward direction ID. The movement of the connection points 1080 in the inward direction ID causes the outer frame portion 1174 to move in the inward direction ID such that the total width of the paddle frame 1124 moves from the expanded width EW (FIG. 109) to the narrowed width NW (FIG. 110). The movement of the paddle frame 1124 to the narrowed position allows the device or implant to more easily maneuver into position for implantation in the heart by reducing the contact and/or friction between the native structures of the heart—e.g., chordae—and the device.

The expanded width EW of the paddle frame 1124 paddle frame 1024 can be between 5 mm and 15 mm, such as between 7 mm and 12 mm, such as between 9 mm and 11 mm, such as about 10 mm. The narrowed width NW of the paddle frame 1124 can be between 3 mm and 12 mm, such as between 5 mm and 10 mm, such as between 7 mm and 9 mm, such as about 8 mm. A ratio of the expanded width EW to the narrowed width NW can be between 10/9 and 3/1, such as between 5/4 and 2/1, such as between 4/3 and 3/2.

While the illustrated example, shows the inner frame portion 1172 of the main support section 1185 having arms 1182 that extend inward from the connection points 1180 and meet at the connection point 1178 such that the inner frame portion 1172 has a triangular shape, it should be understood that the inner frame portion 1072 can take any form that allows the paddle frame 1124 to move to the narrowed position when the tension force F is applied to the paddle frame 1124 such that the paddle frame can allow the device or implant to more easily maneuver into position for implantation in the heart.

In the illustrated example, the inner frame portion 1172 of the main support section 1185 includes arms 1182 that extend inward from the connection points 1180 and meet at the connection point 1178 such that the inner frame portion 1172 has a triangular shape. Referring to FIG. 110, when the anchors of the device or implant move to the open position, the paddle frames 1124 experience a tension force F because the paddle frames 1124 are fixedly connected to the cap and anchors of the device. This tension force F on the paddle frames 1124 cause the connection point 1178 and the connection member 1101 to move in an outward direction OD, which causes the connection points 1180 to move in an inward direction ID. The movement of the connection points 1180 in the inward direction ID cause the outer frame portion 1174 to move in the inward direction ID such that the total width of the paddle frame 1124 moves from the expanded width EW (FIG. 109) to the narrowed width NW (FIG. 110). The movement of the paddle frame 1124 to the narrowed position allows the device or implant to more easily maneuver into position for implantation in the heart by reducing the contact and/or friction between the native structures of the heart—e.g., chordae—and the device.

While the illustrated example, shows the inner frame portion 1172 of the main support section 1185 having arms 1182 that extend inward from the connection points 1180 and meet at the connection point 1178 such that the inner frame portion 1172 has a triangular shape, it should be understood that the inner frame portion 1172 can take any form that allows the paddle frame 1124 to move to the narrowed position when the tension force F is applied to the paddle frame 1124 such that the paddle frame can allow the device or implant to more easily maneuver into position for implantation in the heart.

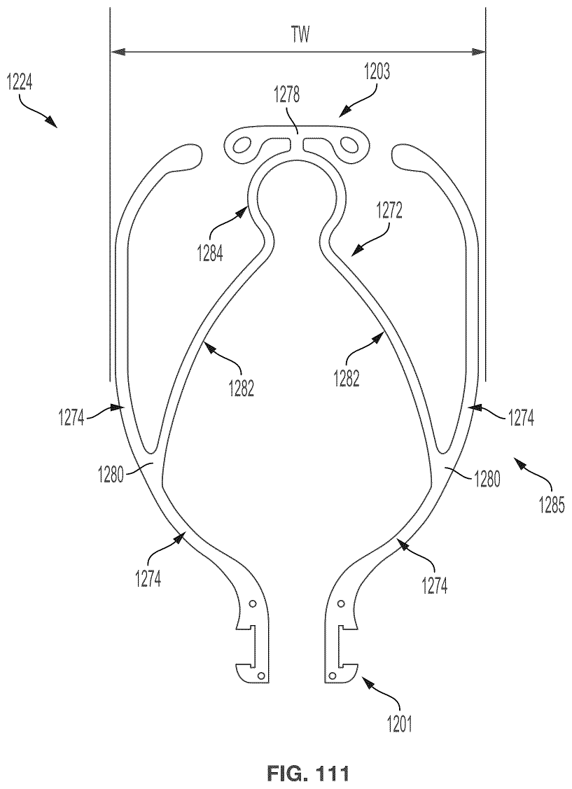

Referring to FIG. 111, an example of a paddle frame 1224 for a device or implant (e.g., device 200 shown in FIGS. 22-37, device 600 shown in FIG. 94, or any other suitable device) includes a main support section 1285, first connection members 1201 (e.g., cutouts, complementary surfaces, connectors, fasteners, etc.) for attaching to a cap of the device or implant, and second connection members 1203 (e.g., cutouts, complementary surfaces, connectors, fasteners, etc.) for attaching to anchors of the device. The paddle frame 1224 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1285 includes an inner frame portion 1272 and an outer frame portion 1274. The inner frame portion 1272 is connected to the connection members 1203 at connection point 1278. The outer frame portion 1274 is connected to the inner frame portion 1272 at connection points 1280, and the outer frame portion 1274 extends to the connection member 1201. The outer frame portion 1274 defines the total width TW of the paddle frame 1224. The inner frame portion 1272 is configured to cause the paddle frame 1224 to move from a normal, expanded position (FIG. 111) when the anchors of the device or implant are in a closed position to a narrowed position when the anchors of the device move to an open position.

In the illustrated example, the inner frame portion 1272 of the main support section 1185 includes arms 1282 and rounded member 1284 (e.g., rounded metal portion, rounded metal wire, etc. . . . ). The arms 1282 extend inward from the connection points 1280, and the rounded member 1284 connects to each of the arms 1282 and connects to the connection point 1278. When the anchors of the device or implant move to the open position, the paddle frames 1224 experience a tension force (e.g., tension force F shown in FIGS. 108 and 110) because the paddle frames 1224 are fixedly connected to the cap and anchors of the device. This tension force F on the paddle frames 1224 cause the connection point 1278 and the connection member 1201 (e.g., cutouts, complementary surfaces, connectors, fasteners, etc.) to move in an outward direction, which causes the connection points 1280 to move in an inward direction. The movement of the connection points 1280 in the inward direction cause the outer frame portion 1274 to move in the inward direction such that the total width TW of the paddle frame 1224 moves to the narrowed position, which allows the device or implant to more easily maneuver into position for implantation in the heart by reducing the contact and/or friction between the native structures of the heart—e.g., chordae—and the device.

The total width TW of the paddle frame 1224 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the total width TW to the narrowed width can be between 10/9 and 3/1, such as between 5/4 and 2/1, such as between 4/3 and 3/2.

While the illustrated example, shows the inner frame portion 1272 of the main support section 1285 having arms 1282 that extend inward from the connection points 1280 and connect to a rounded member 1284 that connects to the connection point 1278, it should be understood that the inner frame portion 1272 can take any form that allows the paddle frame 1224 to move to the narrowed position when the tension force F is applied to the paddle frame 1224 such that the paddle frame can allow the device or implant to more easily maneuver into position for implantation in the heart.

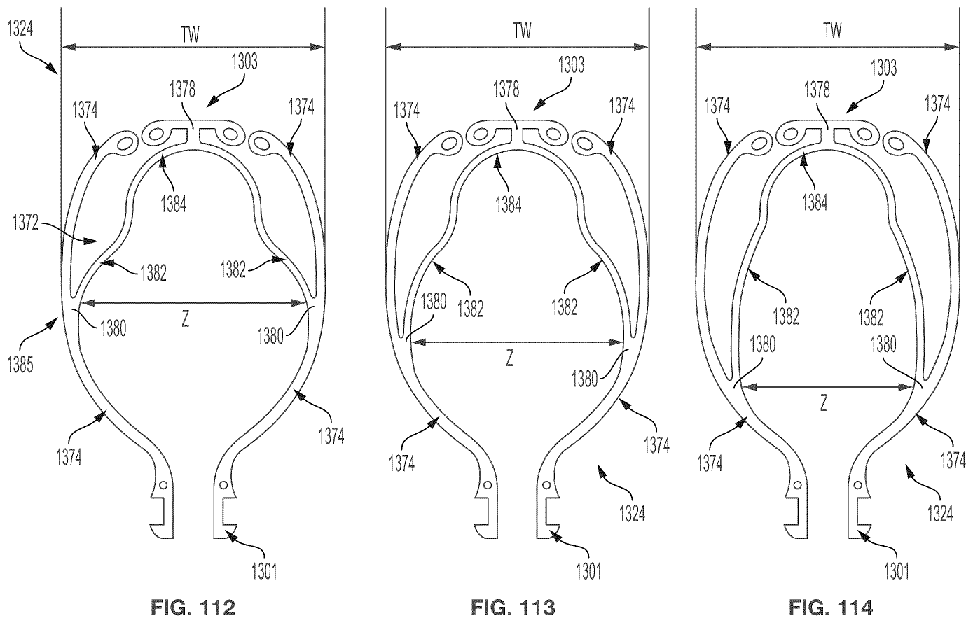

Referring to FIGS. 112-114, an example of a paddle frame 1324 for a device or implant (e.g., device 200 shown in FIGS. 22-37, device 600 shown in FIG. 94, or any other suitable device) includes a main support section 1385, first connection members 1301 (e.g., cutouts, complementary surfaces, connectors, fasteners, etc.) for attaching to a cap of the device or implant, and second connection members 1303 (e.g., projections, cutouts, complementary surfaces, connectors, fasteners, etc.) for attaching to anchors of the device. The paddle frame 1324 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1385 includes an inner frame portion 1372 and an outer frame portion 1374. The inner frame portion 1372 is connected to the connection members 1303 at connection point 1378. The outer frame portion 1374 is connected to the inner frame portion 1372 at connection points 1380, and the outer frame portion 1374 extends to the connection member 1301. The outer frame portion 1374 defines the total width TW of the paddle frame 1324. The inner frame portion 1372 is configured to cause the paddle frame 1324 to move from a normal, expanded position (FIGS. 112-114) when the anchors of the device or implant are in a closed position to a narrowed position when the anchors of the device move to an open position.

In the illustrated example, the inner frame portion 1372 of the main support section 1385 includes arms 1382 and rounded member 1384. The arms 1382 extend inward from the connection points 1380, and the rounded member 1384 connects to each of the arms 1382 and connects to the connection point 1378. The configurations of the paddle frame 1324 shown in FIGS. 112-114 are similar except that connection points 1380 between the inner frame portions 1372 and the outer frame portions 1374 of the paddle frame 1324 are disposed at different locations from the connection member 1301 for each of these configurations. For example, the connection point 1380 for the configuration of the paddle frame 1324 shown in FIG. 112 is further from the connection member 1301 than the configuration of the paddle frame shown in FIG. 113, and the connection point 1380 for the configuration of the paddle frame 1324 shown in FIG. 113 is further from the connection member 1301 than the configuration of the paddle frame shown in FIG. 114. These different configurations cause a width Z between the connection point 1380 to be different for each of these configurations. For example, the width Z for the configuration of the paddle 1324 shown in FIG. 112 is larger than the width Z for the configuration of the paddle 1324 shown in FIG. 113, and the width Z for the configuration of the paddle 1324 shown in FIG. 113 is larger than the width Z for the configuration of the paddle 1324 shown in FIG. 114.

When the anchors of the device or implant move to the open position, the paddle frames 1324 experience a tension force (e.g., tension force F shown in FIGS. 108 and 110) because the paddle frames 1324 are fixedly connected to the cap and the transition portion between the inner and outer paddles of the device. This tension force F on the paddle frames 1324 causes the connection point 1378 and the connection member 1301 to move in an outward direction, which causes the connection points 1380 to move in an inward direction. The movement of the connection points 1380 in the inward direction cause the outer frame portion 1374 to move in the inward direction such that the total width TW of the paddle frame 1324 moves to the narrowed position, which allows the device or implant to more easily maneuver into position for implantation in the heart by reducing the contact and/or friction between the native structures of the heart—e.g., chordae—and the device.

The total width TW of the paddle frame 1324 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the total width TW to the narrowed width can be between 10/9 and 3/1, such as between 5/4 and 2/1, such as between 4/3 and 3/2.

While the illustrated example, shows the inner frame portion 1372 of the main support section 1385 having arms 1382 that extend inward from the connection points 1380 and connect to a rounded member 1384 that connects to the connection point 1378, it should be understood that the inner frame portion 1372 can take any form that allows the paddle frame 1324 to move to the narrowed position when the tension force F is applied to the paddle frame 1324 such that the paddle frame can allow the device or implant to more easily maneuver into position for implantation in the heart.

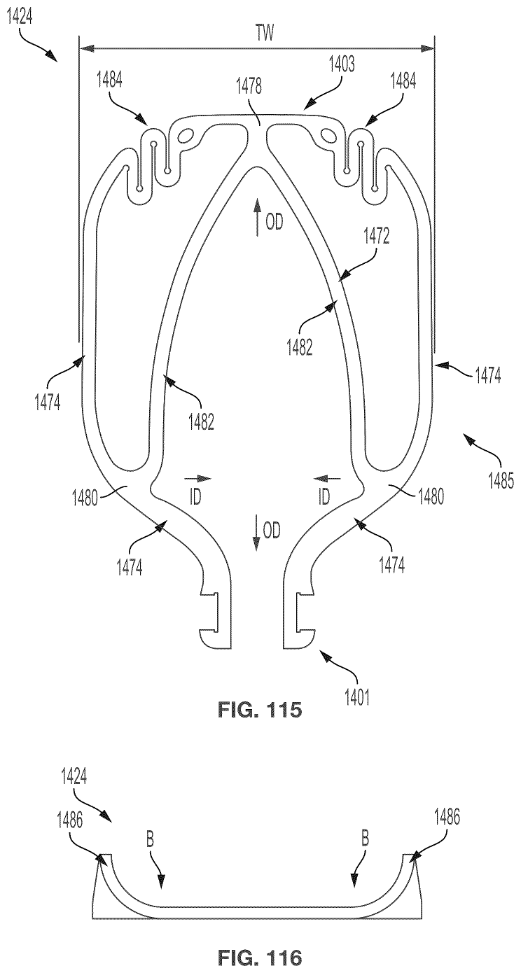

Referring to FIGS. 115-116, an example of a paddle frame 1424 for a device or implant (e.g., device 200 shown in FIGS. 22-37, device 600 shown in FIG. 94, or any other suitable device) includes a main support section 1485, first connection members 1401 (e.g., projections, cutouts, complementary surfaces, connectors, fasteners, etc.) for attaching to a cap of the device or implant, and second connection members 1403 (e.g., projections, cutouts, complementary surfaces, connectors, fasteners, etc.) for attaching to anchors of the device. The paddle frame 1424 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1485 includes an inner frame portion 1472 and an outer frame portion 1474. The inner frame portion 1472 of the main support section 1485 includes arms 1482 that extend inward from the connection points 1480 and connect to the connection point 1478. The outer frame portion 1474 is connected to the inner frame portion 1472 at connection points 1480 and to the connection point 1478 by biasing members 1484 (e.g., springs), and the outer frame portion 1474 extends to the connection member 1401 (e.g., projections, cutouts, complementary surfaces, connectors, fasteners, etc.). The biasing members 1484 cause the at least a portion of the outer frame portion 1474 to bend such that the paddle has curved side edges 1486 (FIG. 16). The curved side edges 1486 can be configured to form against an outer shape of a spacer or coaptation element (e.g., any coaptation element described in the present application) of the device or implant. The biasing members 1484 can be, for example, spring members, or any other member that is capable of causing the paddle frame 1474 to have curved side edges 1486.

The outer frame portion 1474 defines the total width TW of the paddle frame 1424. The inner frame portion 1472 and the biasing members 1484 are configured to cause the paddle frame 1424 to move from a normal, expanded position (FIGS. 115-116) when the anchors of the device or implant are in a closed position to a narrowed position when the anchors of the device move to an open position.

When the anchors of the device or implant move to the open position, the paddle frames 1424 experience a tension force (e.g., tension force F shown in FIGS. 108 and 110) because the paddle frames 1424 are fixedly connected to the cap and the transitions between the inner and outer paddles of the device. This tension force F on the paddle frames 1424 cause the connection point 1478 and the connection member 1401 to move in an outward direction, which causes the connection points 1480 to move in an inward direction. The movement of the connection points 1380 in the inward direction cause the outer frame portion 1474 to move in the inward direction such that the total width TW of the paddle frame 1424 moves to the narrowed position. In addition, the movement of the connection point 1478 in the outward direction causes the biasing members 1484 to cause the curved sided edges 1486 to bend in the direction B (FIG. 116) such that the total width TW of the paddle frame 1424 moves to the narrowed position. The movement of the paddle frame 1424 to the narrowed position allows the device or implant to more easily maneuver into position for implantation in the heart by reducing the contact and/or friction between the native structures of the heart—e.g., chordae and the device.

The total width TW of the paddle frame 1424 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the expanded width TW to the narrowed width can be between 10/9 and 3/1, such as between 5/4 and 2/1, such as between 4/3 and 3/2.

In some implementations, the biasing members 1484 can be passive to allow the anchors of the device or implant to open more when needed, as well as work with the movement of the leaflet and assist in coaptation, when the device or implant is attached to the native leaflets of the heart.

While the illustrated example shows the inner frame portion 1472 of the main support section 1485 having arms 1482 that extend inward from the connection points 1480 and connect to the connection point 1478, it should be understood that the inner frame portion 1472 can take any form that allows the paddle frame 1424 to move to the narrowed position when the tension force F is applied to the paddle frame 1424 such that the paddle frame can allow the device or implant to more easily maneuver into position for implantation in the heart.

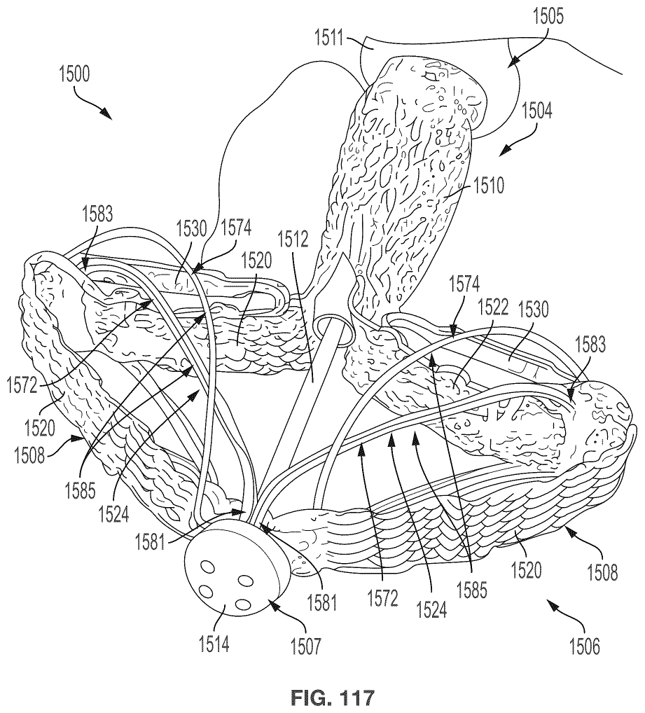

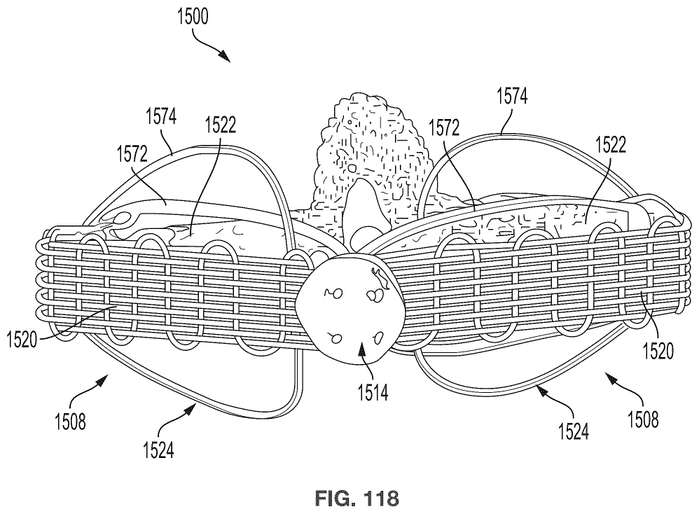

Referring to FIGS. 117-121, an example of a device or implant 1500 includes an anchor portion 1506 having one or more paddle frames 1524. The paddle frames 1524 are configured to allow the device 1500 to maneuver more easily into position for implantation in the heart by reducing the contact and/or friction between the native structures of the heart—e.g., chordae—and the device 1500. That is, the paddle frames 1524 are configured to move between an expanded position (when the device 1500 is in a closed position) and a narrowed position (when the device 1500 is in an open position) and/or the paddle frames can include a flexible outer frame portion that flexes inward to reduce the width of the paddles when the flexible outer frame portion contacts a native heart structure—e.g., chordae.

When the paddle frames 1524 are in the narrowed position, the friction between the native structures of the heart and the device 1500 is reduced. The device 1500 can include any other features for a device or implant discussed in the present application or in the applications and patents incorporated by reference herein, and the device 1500 can be positioned to engage valve tissue 20, 22 as part of any suitable valve repair system (e.g., any valve repair system disclosed in the present application). In addition, any of the devices described herein can incorporate the features of the device 1500.

The device or implant 1500 includes coaptation portion 1504 (e.g., spacer, coaption element, gap filler, etc.), a proximal or attachment portion 1505, an anchor portion 1506, and a distal portion 1507. The coaptation portion 1504, attachment portion 1505, and distal portion 1507 can take any suitable form, such as, for example, the form for these portions of the device 200 shown in FIGS. 22-37, or any other form described in the present application. In some implementations, the coaptation portion 1504 optionally includes a coaptation element 1510 (e.g., a spacer, coaption element, gap filler, etc.) that can be used, for example, for implantation between the leaflets 20, 22 of the native mitral valve MV. The coaptation element, etc. 1510 can take any suitable form, such as, for example, any form described in the present application. In the illustrated example, the coaptation element is made from woven wires.

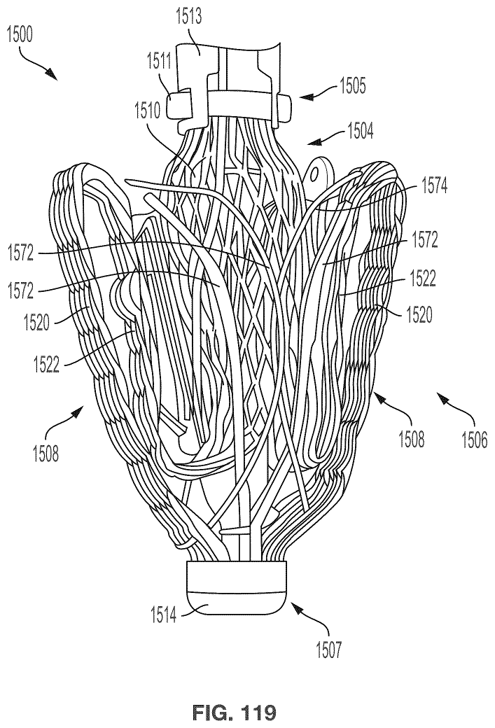

The attachment portion 1505 includes a first or proximal collar 1511 for engaging with a capture mechanism 1513 (FIG. 119) of a delivery system (e.g., the delivery system 502 shown in FIGS. 86A, 87A, 88, and 89). The proximal collar 1511 can take any suitable form, such as, for example, any form described in the present application. The capture mechanism 1513 can take any suitable form, such as, for example, any form described in the present application.

The distal portion 1507 includes a cap 1514 that is attached to anchors 1508 of the anchor portion 1506 such that movement of the cap 1514 causes the anchors 1508 to move between open and closed positions. The cap 1514 can take any suitable form, such as, for example, any form described in the present application. In the illustrated example, an actuation element 1512 (e.g., an actuation wire, actuation shaft, etc.) extends from a delivery system (e.g., any delivery system described in the present application) and engages the cap 1514 to move the cap 1514 relative to the coaptation element or spacer 1510 to enable actuations of the device 1500. The actuation element 1512 can engage and move the cap by any suitable means, such as, for example, any means provided in the present application.

The anchor portion 1506 of the device 1500 can take any suitable form, such as, for example, the form of the anchor portion 206 of the device 200 shown in FIGS. 22-37 (except that the paddle frame 224 is replaced with the paddle frame 1524 shown in FIGS. 91-95 and described in more detail below), or any other form described in the present application that can incorporate paddle frame 1524. The anchor portion 1506 can include a plurality of anchors 1508, each anchor 1508 including outer paddles 1520, inner paddles 1522, paddle extension members or paddle frames 1524, and clasps 1530.

The paddle frame 1524 includes a main support section 1585, first connection members (e.g., connection members 601 shown in FIGS. 91-95 or any other connection members described in the present application) for attaching to a cap of the device or implant, and second connection members (connection members 603 shown in FIGS. 91-95 or any other connection members described in the present application) for attaching to anchors of the device. The paddle frame 1524 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

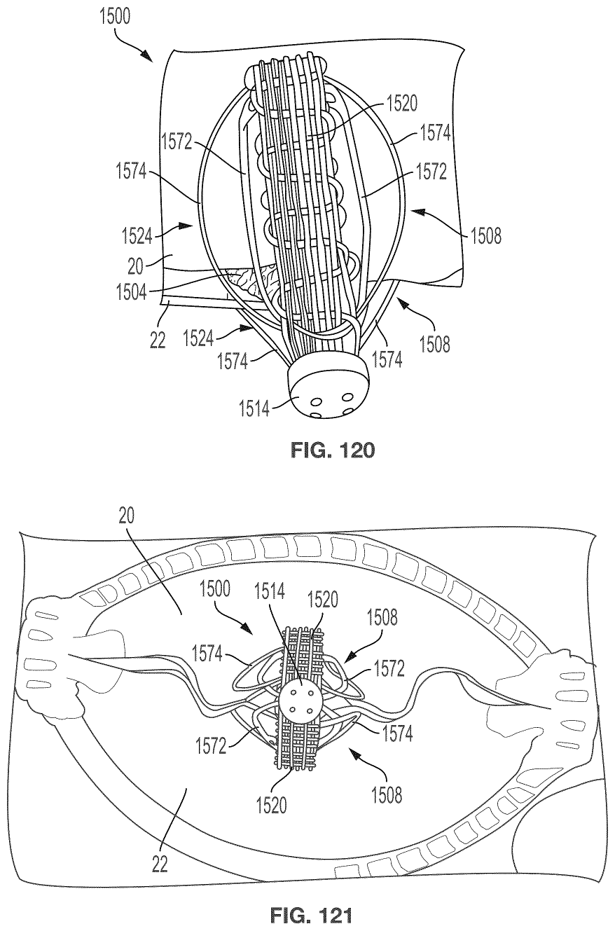

The main support section 1585 includes a rigid inner frame portion 1572 and a flexible outer frame portion 1574. The rigid inner frame portion 1572 has a first end 1581 that connects to the cap 1514 and a second end 1583 that connects to the anchors 1508. Referring to FIGS. 120 and 121, the rigid inner frame portion is configured to support the paddles 1520, 1522 of the anchors and provide a sufficient force to facilitate coaptation of the native leaflets 20, 22 against the coaptation element 1510 when the anchors 1508 are in the closed position. The rigid inner frame portion 1572 can be made of, for example, metals, plastics, etc.

Referring again to FIGS. 117-121, the flexible outer frame portion 1574 is connected to the rigid inner frame portion and defines the total width of the paddle frame 1524. That is, the flexible outer frame portion 1574 has a greater total width than the rigid inner frame portion 1572. The flexible outer frame portion 1574 is configured such that forces (e.g., forces from the flexible outer frame portion 1574 contacting the chordae during implantation of the device 1500) cause the flexible outer frame portion 1574 to flex and allow the device 1500 to maneuver more easily into position for implantation in the heart. Referring to FIGS. 120 and 121, when the anchors 1508 are in the closed position and causing leaflets to coapt against the coaptation element 1510, the flexible outer frame portion 1574 maintains its normal total width to provide for a larger surface area (relative to the rigid inner frame portion 1572) contacting the leaflets to hold the leaflets against the coaptation element 1510. The flexible outer frame portion 1574 can be made of, for example, metals, and plastics.

The total width of the flexible outer frame portion 1574 can be 5 mm and 15 mm, such as between 7 mm and 12 mm, such as between 9 mm and 11 mm, such as about 10 mm. The width of the inner frame portion 1572 can be between 2 mm and 8 mm, such as between 4 mm and 6 mm, such as about 5 mm.

In some implementations, the flexible outer frame portion 1574 are shaped set inward such that the total width of the outer frame portion 1574 narrows when the anchors 1508 are in the open position, and such that the outer frame portion moves back to its normal total width when the anchors 1508 are moved to the closed position.

While the illustrated example, shows rigid inner frame portion 1572 and the flexible outer frame portion 1574 having rounded shapes, it should be understood that the inner and outer frame portions 1572, 1574 can take any form that allows the device 1500 to more easily maneuver into position for implantation in the heart while providing sufficient support for facilitating coaptation of the leaflets of a native heart valve against the coaptation element 1510.

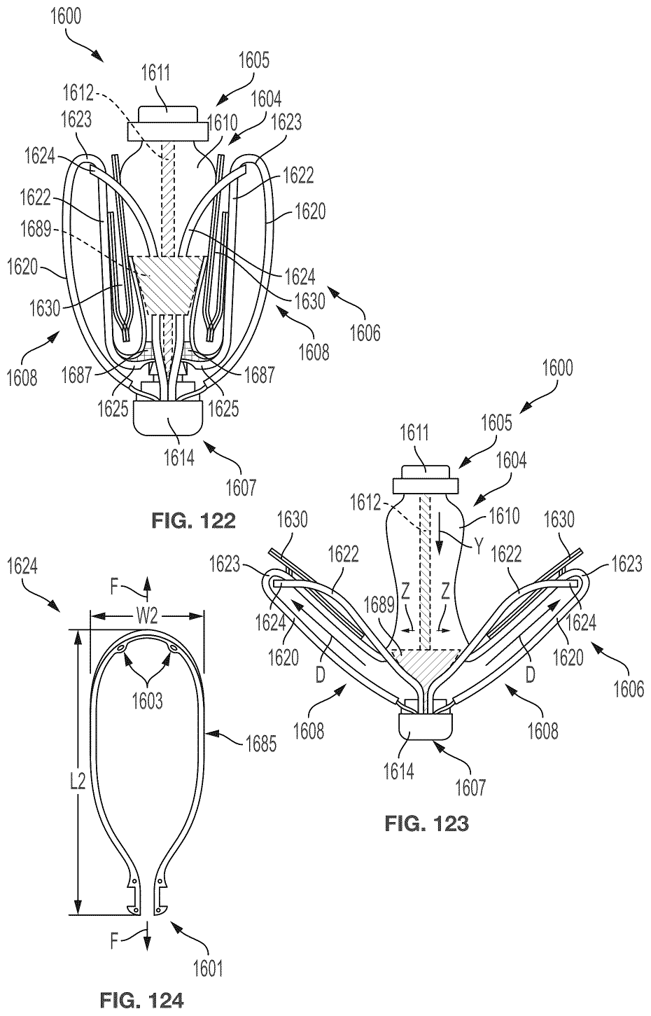

Referring to FIGS. 122-124, an example implementation of a device or implant 1600 includes a coaptation portion 1604, a proximal or attachment portion 1605, an anchor portion 1606, and a distal portion 1607. The device or implant 1600 is configured such that a paddle frame 1624 of the anchor portion 1606 is moved to a narrowed position (FIG. 124) to allow the device 1600 to maneuver more easily into position for implantation in the heart by reducing the contact and/or friction between the native structures of the heart—e.g., chordae and the device 1600. That is, the paddle frames 1624 are configured to move between an expanded position when the device 1600 is in a closed position (FIG. 122) and a narrowed position when the device 1600 is in an open position (FIG. 123)), when the paddle frames 1624 are in the narrowed position, the contact between the native structures of the heart and the device 1600 is reduced. The device 1600 can include any other features of devices or implants discussed elsewhere in the present application or in the applications and patents incorporated by reference herein, and the device 1600 can be positioned to engage valve tissue 20, 22 as part of any suitable valve repair system (e.g., any valve repair system disclosed in the present application). In addition, any of the devices described herein can incorporate the features of the device 1600.

The attachment portion 1605 includes a first or proximal collar 1611 for engaging with a capture mechanism (e.g., the capture mechanism 213 shown in FIGS. 44-49) of a delivery system (e.g., the delivery system 202 shown in FIGS. 38-49). The proximal collar 1611 can take any suitable form, such as, for example, any form described in the present application. The distal portion 1607 includes a cap 1614 that is attached to outer paddles 1620 of the anchors 1608 such that movement of the cap 1614 causes the anchors 1608 to move between open and closed positions. The cap 1614 can take any suitable form, such as, for example, any form described in the present application.

The anchor portion 1606 can take any suitable form, such as, for example, the form of the anchor portion 206 of the device 200 shown in FIGS. 22-37, or any other form described in the present application that can incorporate paddle frame 1524. The anchor portion 1606 can include a plurality of anchors 1608, each anchor 1608 including outer paddles 1620, inner paddles 1622, paddle extension members or paddle frames 1624, and clasps 1630. In the illustrated example, the inner paddles 1622 are made from a material that has a greater stiffness than the material of the outer paddles 1620.

Referring to FIG. 124, the paddle frame 1624 includes a main support section 1685, first connection members 1601 (e.g., projections, cutouts, complementary surfaces, connectors, fasteners, etc.) for attaching to a cap 1614 of the device 1600, and second connection members 1603 (e.g., projections, cutouts, complementary surfaces, connectors, fasteners, etc.) for attaching to the connection portion 1623 between the inner paddle 1622 and the outer paddle 1620 of the anchors 1608. The paddle frame 1624 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness. The paddle frames 1624 can, however, take any other suitable form, such as, for example, any form described in the present application.

In some implementations, the coaptation portion 1604 includes a coaptation element 1610 (e.g., a coaptation element 1610 that can be used, for example, for implantation between the leaflets 20, 22 of the native mitral valve MV). The coaptation element 1610 (e.g., spacer, coaption element, gap filler, etc.) can take any suitable form, such as, for example, any form described in the present application. The coaptation element 1610 is connected to the connection portions 1623 of the inner paddles 1622 of the anchors 1608. In the illustrated example, the coaptation element 1610 includes one or more flexible portions 1687 that connect to the inner paddles 1622. The flexible portions 1687 can be formed from a woven material having a looser weave than the inner paddle portion and/or the remainder of the coaptation element 1610, can be formed from an elastic material that is more elastic than the inner paddle portion and/or the remainder of the coaptation element 1610, or can be formed in any other way that makes the flexible portions 1687 more flexible and/or stretchable than the inner paddle portion and/or the remainder of the coaptation element 1610.

During implantation, the paddles 1620, 1622 of the anchors 1608 are opened and closed to grasp the native valve leaflets between the paddles 1620, 1622 and the coaptation element 1610. The anchors 1608 are moved between a closed position (FIG. 122) to various open positions (e.g., the position shown in FIG. 123) by extending and retracting an actuation element 1612 (e.g., an actuation wire, actuation shaft, etc.). Extending and retracting the actuation element 1612 increases and decreases the spacing between the coaptation element 1610 and the cap 1614, respectively. The proximal collar 1611 and the coaptation element 1610 slide along the actuation element 1612 during actuation so that changing of the spacing between the coaptation element 1610 and the cap 1614 causes the paddles 1620, 1622 to move between different positions to grasp the native valve leaflets during implantation.

In the illustrated example the actuation element 1612 includes a wide portion 1689 for facilitating movement of the paddle frame 1624 to the narrowed position. Referring to FIG. 123, as the actuation element 612 is moved downward through the coaptation element 610 in the direction Y to move the anchors 1608 from the closed position to the open position, the wide portion 1689 of the actuation element 1612 engages the flexible portion 1687 of the coaptation element 1610, which causes the flexible portion 1687 to move in an outward direction Z. This movement of the flexible portion 1687 in the outward direction Z causes the connection portions of the inner paddles 1622 to move in the outward direction D relative to the cap 1614, which causes the connection portion 1623 (which the paddle frame 1624 is connected to) of the anchors 1608 to also move in the direction D. The rigid stiffness of the inner paddle 1622 helps facilitate movement of the connection portion 1623. Because the paddle frame 1624 is connected to the cap 1614 and the connection portion 1623 of the anchor 1608, this movement of the connection portion 1623 in the direction D causes a tensioning force F (FIG. 124) on the paddle frame 1624, which causes the paddle frame 1624 to move to the narrowed position (FIG. 124). In the illustrated example, the wide portion 1689 of the actuation element 1612 has a tapered shape for engaging the flexible portions 1687 of the coaptation element 1610 to facilitate movement of the paddle frame 1624 to the narrowed position. In some implementations, the wide portion 1689 can have a spherical shape, or any other suitable shape.

In some implementations, the wide portion 1689 is configured to only widen the transition portion 1625 for a small portion of the travel of the actuation element. For example, the path of travel of the actuation element can have a path of travel a beginning that corresponds to the device being fully closed and an end that corresponds to the device being fully closed. The wide portion 1689 can be configured to leave the transition portion 1625 at its original width along a beginning of the path of travel, move the transition portion 1625 to a wider width during a middle of the path of travel, and allow the transition portion 1625 to move back to its original width along an end portion of the path of travel.

Referring to FIG. 124, the paddle frames 1624 have a length L2 and a total width W2 when in the narrowed position. The width of the paddle frame 1424 when in the normal, expanded position can be between 5 mm and 15 mm, such as between 7 mm and 12 mm, such as between 9 mm and 11 mm, such as about 10 mm. The narrowed width W2 of the paddle frame 1124 can be between 3 mm and 12 mm, such as between 5 mm and 10 mm, such as between 7 mm and 9 mm, such as about 8 mm. A ratio of the normal width to the narrowed width W2 can be between 10/9 and 3/1, such as between 5/4 and 2/1, such as between 4/3 and 3/2.

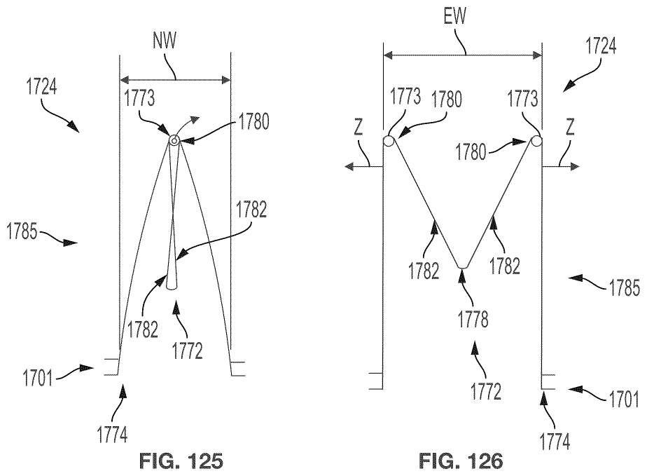

Referring to FIGS. 125 and 126, an example of a paddle frame 1724 for a device or implant (e.g., device 200 shown in FIGS. 22-37, device 600 shown in FIG. 94, or any other suitable device) includes a main support section 1785, first connection members 1701 (e.g., projections, cutouts, complementary surfaces, connectors, fasteners, etc.) for attaching to a cap of the device or implant, and second connection members (e.g., connection members 603 shown in FIGS. 91-95 or any other connection members described in the present application) for attaching to anchors of the device. The paddle frame 1724 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

The main support section 1785 includes an inner frame portion 1772 and an outer frame portion 1774. The inner frame portion 1772 is connected to the outer frame portion 1774 at connection points 1780, and the outer frame portion 1774 extends to the connection member 1701 (e.g., projections, cutouts, complementary surfaces, connectors, fasteners, etc.). In the illustrated example, the inner frame portion 1772 includes arms 1782 that extend inward from each connection point 1780 and meet at connection portion 1778 such that the arms 1782 have a V-shape when the paddle frame 1724 is in the expanded position (FIG. 126). The connection portions 1780 between the inner and outer frame portions 1772, 1774 can include an opening 1773 for receiving a holding device (e.g., a suture, pin, or other suitable device) for connecting the openings 1773 together to maintain the paddle frame 1724 in the narrow position (FIG. 125). When the holding device is removed from the openings 1773 such that the openings 1773 are no longer connected, the paddle frame 1724 is configured to move outward in the direction Z to its normal, expanded position. That is, the paddle frame 1724 can be made of a material that is preformed into the expanded position (as shown in FIG. 126). A holding device can be used to connect the openings 1773 at the connection points 1780 such that the paddle frame 1724 is maintained in a folded, narrow position (as shown in FIG. 125). Removal of the holding device then causes the paddle frame 1724 to spring back to the normal, expanded position.

During movement of the device or implant to a position to be implanted on native valve leaflets (e.g., mitral valve leaflets 20, 22, tricuspid leaflets 30, 32, 34, or other valve leaflets) of a patient, the paddle frame 1724 is maintained in the narrowed position to allow the device or implant to more easily maneuver into position for implantation in the heart by reducing the contact and/or friction between the native structures of the heart—e.g., chordae—and the device. Once the device is positioned for implantation, the holding device is removed from the openings 1773 such that the paddle frame 1724 moves to the expanded position such that the anchors of the device have a larger surface area for capturing the leaflets of the native valve.

The outer frame portion 1774 defines the total width (e.g., the expanded width EW shown in FIG. 126 and the narrowed width NW shown in FIG. 125) of the paddle frame 1724. The expanded width EW of the paddle frame 1424 when in the normal, expanded position can be between 5 mm and 15 mm, such as between 7 mm and 12 mm, such as between 9 mm and 11 mm, such as about 10 mm. The narrowed width NW of the paddle frame 1124 can be between 3 mm and 12 mm, such as between 5 mm and 10 mm, such as between 7 mm and 9 mm, such as about 8 mm. A ratio of the normal width to the narrowed width W2 can be between 10/9 and 3/1, such as between 5/4 and 2/1, such as between 4/3 and 3/2.

While the illustrated example, shows the inner frame portion 1772 of the main support section 1785 having arms 1782 that make a V-shape, it should be understood that the inner frame portion 1772 can take any form that allows the paddle frame 1724 to be folded into and maintained in a narrow position when engaged by a holding device, and also allows the paddle frame 1724 to move to the expanded position upon disengaging the holding device from the paddle frame 1724.

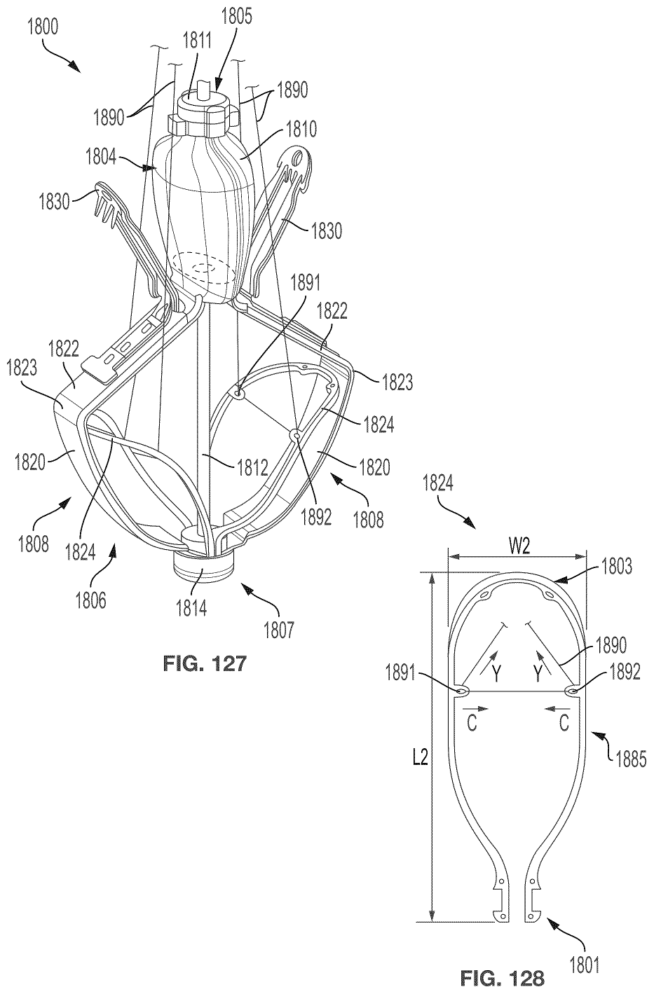

Referring to FIGS. 127-130, an example implementation of a device or implant 1800 includes an anchor portion 1806 having one or more paddle frames 1824 that are movable to a narrowed position to allow the device 1800 to maneuver more easily into position for implantation in the heart by reducing the contact and/or friction between the native structures of the heart—e.g., chordae—and the device 1800. That is, width adjust adjustment elements (e.g., width adjustment wire, width adjustment shaft, width adjustment tube, width adjustment line, width adjustment cord, width adjustment suture, width adjustment screw or bolt etc.), such as the illustrated lines 1890 are controlled by a user to create a compression force C (FIG. 128) on the paddle frames 1824 to move the paddle frames 1824 to a narrowed position as the device 1800 is being positioned for implantation on the native leaflets of a native valve such that the contact and/or friction between the native structures of the heart and the device 1800 is reduced. The device 1800 can include any other features of devices or implants discussed elsewhere in the present application or in the applications and patents incorporated by reference herein, and the device 1800 can be positioned to engage valve tissue 20, 22 as part of any suitable valve repair system (e.g., any valve repair system disclosed in the present application). In addition, any of the devices described herein can incorporate the features of the device 1800.

The device or implant 1800 includes a coaptation portion 1804, a proximal or attachment portion 1805, an anchor portion 1806, and a distal portion 1807. The coaptation portion 1804, attachment portion 1805, and distal portion 1807 can take any suitable form, such as, for example, the form for these portions of the device 200 shown in FIGS. 22-37, or any other form described in the present application. In some implementations, the coaptation portion 1804 includes coaptation element 1810 (e.g., a spacer, coaption element, gap filler, etc.) that can be used, for example, for implantation between the leaflets 20, 22 of the native mitral valve MV. The coaptation element 1810 can take any suitable form, such as, for example, any form described in the present application.

The attachment portion 1805 includes a first or proximal collar 1811 for engaging with a capture mechanism (e.g., the capture mechanism 213 shown in FIGS. 44-49) of a delivery system (e.g., the delivery system 202 shown in FIGS. 38-49). The proximal collar 1811 can take any suitable form, such as, for example, any form described in the present application.

The distal portion 1807 includes a cap 1814 that is attached to anchors 1808 of the anchor portion 1806 such that movement of the cap 1814 causes the anchors 1508 to move between open and closed positions. The cap 1814 can take any suitable form, such as, for example, any form described in the present application. In the illustrated example, an actuation element 1812 (e.g., an actuation wire, an actuation shaft, etc.) extends from a delivery system (e.g., any delivery system described in the present application) and engages the cap 1814 to move the cap 1814 relative to the coaptation element or spacer 1810 to enable actuations of the device 1800. The actuation element 1812 can engage and move the cap by any suitable means, such as, for example, any means provided in the present application.

The anchor portion 1806 can take any suitable form, such as, for example, the form of the anchor portion 206 of the device 200 shown in FIGS. 22-37 or any other form described in the present application. The anchor portion 1806 can include a plurality of anchors 1808, each anchor 1808 including outer paddles 1820, inner paddles 1822, paddle extension members or paddle frames 1824, and clasps 1830. The paddle frames 1824 can include a main support section 1885, first connection members (e.g., projections, cutouts, complementary surfaces, connectors, fasteners, etc.) for attaching to the cap 1814, and second connection members (e.g., projections, cutouts, complementary surfaces, connectors, fasteners, etc.) for attaching to a connection portion 1823 of the anchors 1808. The paddle frame 1824 can attach to the connection portion of the anchors and the cap by any suitable means, such as, for example, any means described in the present application. The thickness and width of the paddle frame 1824 can take any suitable form, such as, for example, the thickness can be substantially identical to the width, the thickness can be greater than the width (as shown in FIGS. 91-95), or the width can be greater than the thickness.

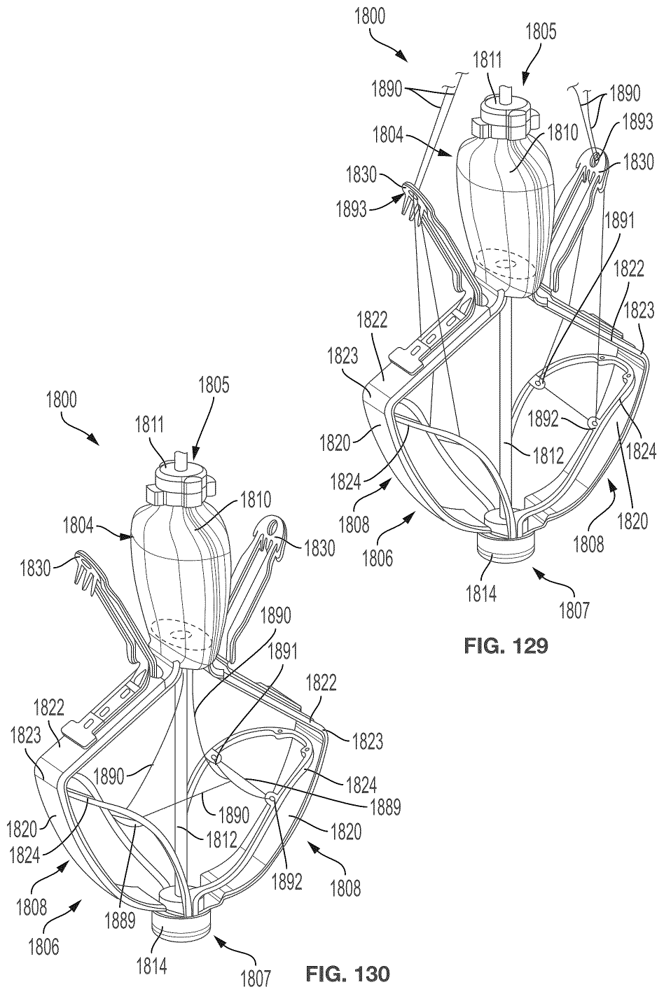

The paddle frame 1824 includes an end 1801 that is configured to be attached to the cap 1814 and a free end 1803. The paddle frame 1824 includes a first opening 1891 and a second opening 1892 for receiving one or more lines 1890 of the delivery system. Referring to FIGS. 127-129, in some examples, a single line 1890 extends through the first and second openings 1891, 1892 of each paddle frame 1824 and into the delivery system such that a user can pull the lines 1890 to cause the paddle frame 1824 to move to the narrowed position. Referring to FIG. 129, the lines 1890 can also extend through an opening 1893 of the clasp 1830 of each anchor 1808 before extending into the delivery system. Referring to FIG. 128, when a user pulls the line 1890, a force is created on each end of the line 1890 in the direction Y, which causes a compression force C on the paddle frame 1824 due to the line extending through the openings 1891, 1892. The compression force C causes the paddle frame 1824 to move to the narrowed position.

Referring to FIG. 129, the lines 1890 can also extend through an opening 1893 of the clasp 1830 of each paddle before extending into the delivery system. When a user pulls the line 1890, the clasps 1830 are opened and the paddle frame 1824 to moves to the narrowed position.

Referring to FIG. 130, in some examples, a connection line 1889 extends in a closed loop between the first and second openings 1891, 1892 of each paddle frame 1824, and a single line 1890 extends through the closed loop of each connection line 1889 such that a user only needs to pull the single line 1890 to move both paddle frames 1824 to the narrowed position. That is, pulling the single line 1890 creates a compression force (e.g., similar to compression force C shown in FIG. 128) on each of the paddle frames 1824 simultaneously that causes the paddle frames 1824 to move to the narrowed position. In the illustrated example, the single line 1890 extends through the coaptation element 1810 prior to extending into the delivery system. Referring to FIGS. 127-130, the lines 1889, 1890 can be, for example, sutures.

Referring to FIG. 128, the paddle frames 1824 have a length L2 and a total width W2 when in the narrowed position. The width of the paddle frame 1424 when in the normal, expanded position can be between 5 mm and 15 mm, such as between 7 mm and 12 mm, such as between 9 mm and 11 mm, such as about 10 mm. The narrowed width W2 of the paddle frame 1124 can be between 3 mm and 12 mm, such as between 5 mm and 10 mm, such as between 7 mm and 9 mm, such as about 8 mm. A ratio of the normal width to the narrowed width W2 can be between 10/9 and 3/1, such as between 5/4 and 2/1, such as between 4/3 and 3/2. While the dimensions described above for the paddle frame 1824 in the narrowed and expanded position are made with reference to the examples shown in FIGS. 127-129, it should be understood that the same dimensions can apply to the example shown in FIG. 130.

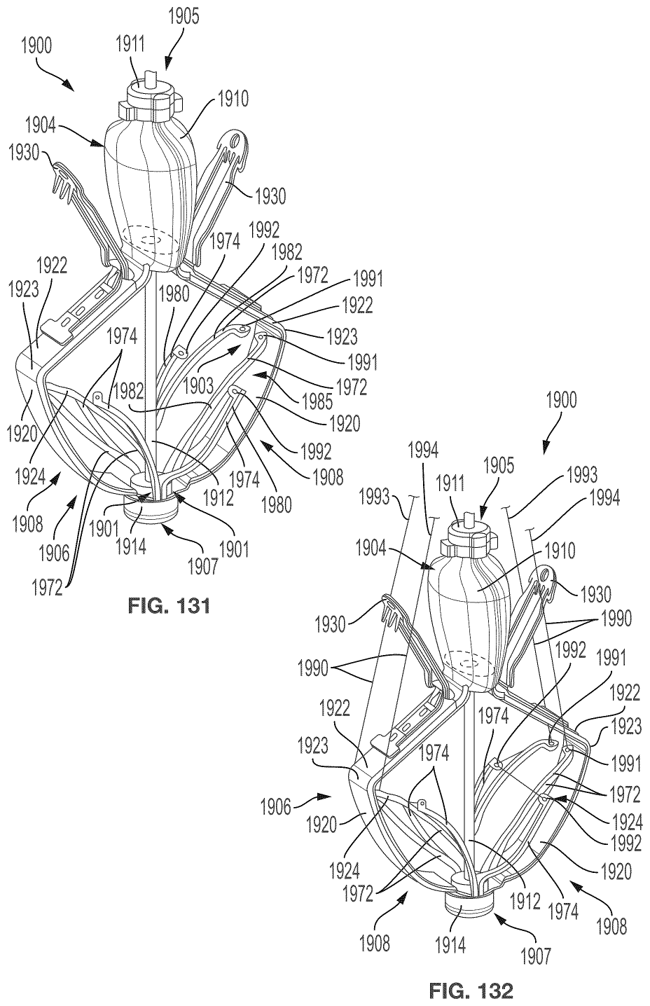

Referring to FIGS. 131-136, an example implementation of a device or implant 1900 includes an anchor portion 1906 having one or more paddle frames 1924 that are movable to a narrowed position to allow the device 1900 to maneuver more easily into position for implantation in the heart by reducing the contact and/or friction between the native structures of the heart—e.g., chordae—and the device 1900. That is, lines 1990 are controlled by a user to create a compression force (e.g., similar to compression force C in FIG. 128) on the paddle frames 1924 to move the paddle frames 1924 to a narrowed position as the device 1900 is being positioned for implantation on the leaflets of a native valve such that the contact between the native structures of the heart and the device 1900 is reduced. The device 1900 can include any other features of devices or implants discussed elsewhere in the present application or in the applications and patents incorporated by reference herein, and the device 1900 can be positioned to engage valve tissue (e.g., leaflets 20, 22, etc.) as part of any suitable valve repair system (e.g., any valve repair system disclosed in the present application). In addition, any of the devices described herein can incorporate the features of the device 1900.

The device or implant 1900 includes a coaptation portion 1904, a proximal or attachment portion 1905, an anchor portion 1906, and a distal portion 1907. The coaptation portion 1904, attachment portion 1905, and distal portion 1907 can take any suitable form, such as, for example, the form for these portions of the device 200 shown in FIGS. 22-37, or any other form described in the present application. In some implementations, the coaptation portion 1904 includes a coaptation element 1910 (e.g., spacer, coaption element, gap filler, etc.) that can be used, for example, for implantation between the leaflets 20, 22 of the native mitral valve MV. The coaptation element 1910 can take any suitable form, such as, for example, any form described in the present application.

The attachment portion 1905 includes a first or proximal collar 1911 for engaging with a capture mechanism (e.g., the capture mechanism 213 shown in FIGS. 44-49) of a delivery system (e.g., the delivery system 202 shown in FIGS. 38-49). The proximal collar 1911 can take any suitable form, such as, for example, any form described in the present application.

The distal portion 1907 includes a cap 1914 that is attached to anchors 1908 of the anchor portion 1906 such that movement of the cap 1914 causes the anchors 1908 to move between open and closed positions. The cap 1914 can take any suitable form, such as, for example, any form described in the present application. In the illustrated example, an actuation element 1912 (e.g., an actuation wire, actuation shaft, etc.) extends from a delivery system (e.g., any delivery system described in the present application) and engages the cap 1914 to move the cap 1914 relative to the coaptation element 1910 to enable actuations of the device 1900. The actuation element 1912 can engage and move the cap by any suitable means, such as, for example, any means provided in the present application.

The anchor portion 1906 can take any suitable form, such as, for example, the form of the anchor portion 206 of the device 200 shown in FIGS. 22-37 or any other form described in the present application. The anchor portion 1906 can include a plurality of anchors 1908, each anchor 1908 including outer paddles 1920, inner paddles 1922, paddle extension members or paddle frames 1924, and clasps 1930. The paddle frames 1924 can include a main support section 1985, first connection members 1901 (e.g., projections, cutouts, complementary surfaces, connectors, fasteners, etc.) for attaching to the cap 1914, and second connection members 1903 (e.g., projections, cutouts, complementary surfaces, connectors, fasteners, etc.) for attaching to a connection portion 1923 of the anchors 1908. The paddle frame 1924 can attach to the connection portion of the anchors and the cap by any suitable means, such as, for example, any means described in the present application. The thickness and width of the paddle frame 1924 can take any suitable form, such as, for example, the thickness can be substantially identical to the width, the thickness can be greater than the width (as shown in FIGS. 91-95), or the width can be greater than the thickness.

The main support section 1985 includes an inner frame portion 1972 and an outer frame portion 1974 that are connected to the connection members 1901. In the illustrated example, the inner frame portion 1972 has a pair of arms 1982 that extend from the connection members 1901 to the connection members 1903, where the arms 1982 provide support for the anchors 1908. The outer frame portion 1974 has a pair of arms 1980 that extend from the connection members 1901 in an outward direction relative to the arms 1982 of the inner frame portion 1972 such that the arms 1980 define the total width (e.g., the total width W2 shown in FIG. 128) of the paddle frame 1924. Referring to FIG. 131, the arms 1980 each include an opening 1992 for receiving one or more lines 1990 (FIGS. 132-136) of the delivery system. The arms 1982 can also include openings 1991 for receiving the one or more lines 1990. The openings 1991 can be the openings of the connection member 1903 for connecting to the connection portion 1923 of the anchors 1908 (as shown in the illustrated example), or the openings 1991 can be separate from the connection member 1903.

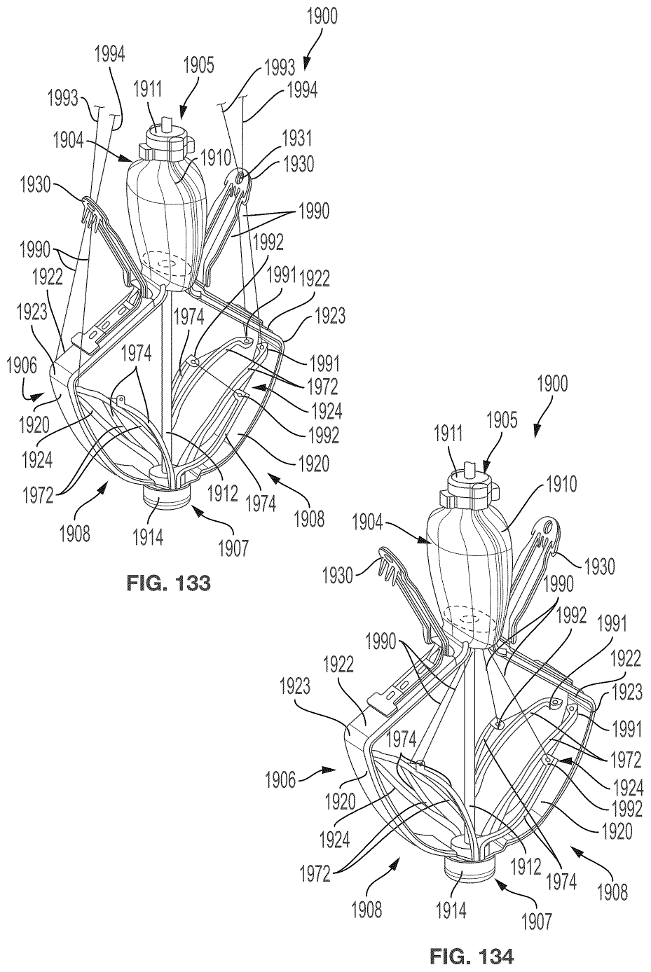

Referring to FIGS. 132 and 133, in some examples, a single line 1990 corresponds to each paddle frame 1924 to move the paddle frame 1924 to the narrowed position. In the illustrated example, the lines 1990 extend through the openings 1991, 1992 of the paddle frames 1924 such that a user can cause a pulling force on the lines 1990 to move the paddle frames 1924 to the narrowed position. Each line 1990 has a first end 1993 and a second end 1994. The first end 1993 extends from the delivery system, through the opening 1991 of one arm 1982 of the inner frame portion 1972, through the opening 1992 of one arm 1980 of the outer frame portion 1974, through the opening 1992 of the other arm 1980 of the outer frame portion 1974, through the opening 1991 of the other arm 1982 of the inner frame portion 1972, and the second end 1994 of the line 1990 extends into the delivery system. In the example shown in FIG. 132, a pulling force on both ends 1993, 1994 of the line 1990 causes the paddle frame to move to the narrowed position by creating a compression force on the arms 1980 (FIG. 131) of the outer frame portion 1974 causing the arms 1980 to move toward the inner frame portion 1972 of the paddle frame 1924.

Referring to FIG. 133, the lines 1990 can also extend through an opening 1931 of the clasp 1930 of each paddle before extending into the delivery system. In examples shown in FIG. 132, a pulling force on both ends 1993, 1994 of the line 1990 causes the paddle frame to move to the narrowed position and opens the clasp.

Referring to FIG. 134, a single line 1990 corresponds to each paddle frame 1924 to move the paddle frame 1924 to the narrowed position. Each line 1990 has a first end (not shown) that extends from the delivery system, through the coaptation element 1910, through the opening 1992 of one arm 1980 of the outer frame portion 1974, through the opening 1991 of one arm 1982 of the inner frame portion 1972, through the opening 1991 of the other arm 1982 of the inner frame portion 1972, through the opening 1992 of the other arm 1980 of the outer frame portion 1974, and the second end (not shown) of the line 1990 extends through the coaptation element 1910 and into the delivery system. A pulling force on both ends of the line 1990 causes the paddle frame 1924 to move to the narrowed position by creating a compression force on the arms 1980 (FIG. 131) of the outer frame portion 1974 causing the arms 1980 to move toward the inner frame portion 1972 of the paddle frame 1924.

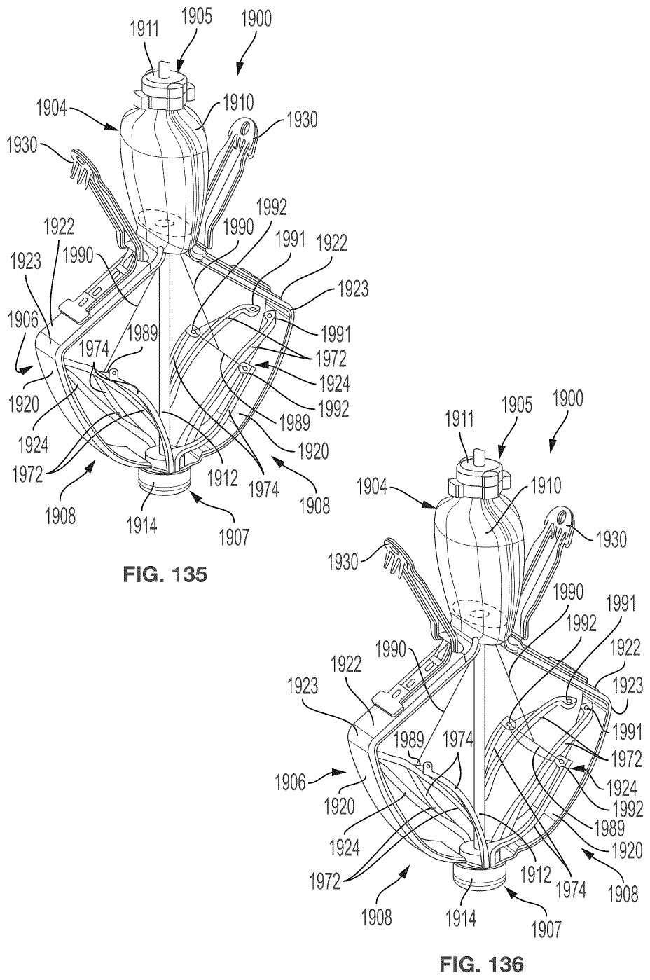

Referring to FIGS. 135 and 136, in some implementations, a connection line 1989 is connected to each paddle frame 1924, and a line 1990 is connected to the connection line 1989 such that a user can pull the line 1990 to move the paddle frame 1924 to the narrowed position. Referring to FIG. 135, in some implementations, the connection line 1989 extends in a closed loop between the openings 1991, 1992 of the inner frame portion 1972 and the outer frame portion 1974 of the paddle frame 1924. Referring to FIG. 136, in some implementations, the connection line 1989 extends in a closed loop between the openings 1992 of the outer frame portion 1974 of the paddle frame 1924, but the connection line 1989 does not extend through the openings 1991 of the inner frame portion 1972.

In both of the examples shown in FIGS. 135 and 136, a pulling force on the lines 1990 causes the paddle frame to move to the narrowed position by creating a compression force on the arms 1980 (FIG. 131) of the outer frame portion 1974 causing the arms 1980 to move toward the inner frame portion 1972 of the paddle frame 1924. While the examples shown in FIGS. 135 and 136 show a separate line 1990 being attached to the actuation connection line 1989 of each paddle frame 1924, in some implementations, a single line (e.g., similar to the line 1890 shown in FIG. 130) can be attached to the connection line 1989 of each paddle frame 1924 such that pulling the ends of the single line causes both paddle frames 1924 to move to the narrowed position. Referring to FIGS. 131-136, the lines 1989, 1990 can be, for example, sutures.

Still referring to FIGS. 131-136, the total width of the paddle frame 1924 (defined by the outer frame portion 1974 of the paddle frame 1924) when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the normal width to the narrowed width can be between 10/9 and 3/1, such as between 5/4 and 2/1, such as between 4/3 and 3/2.

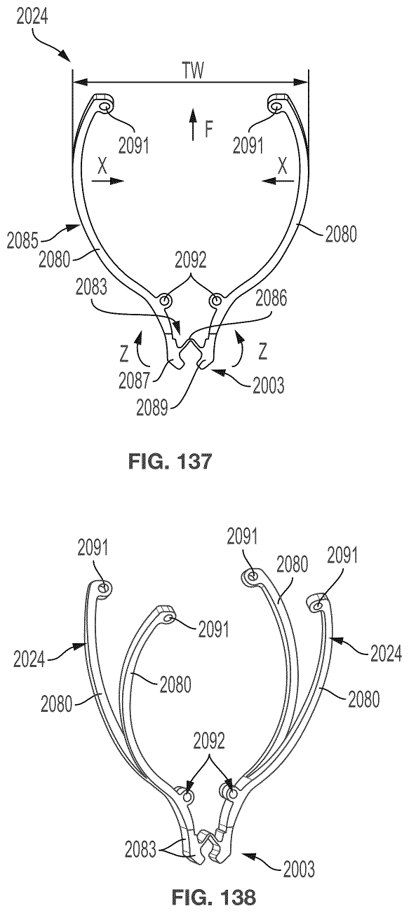

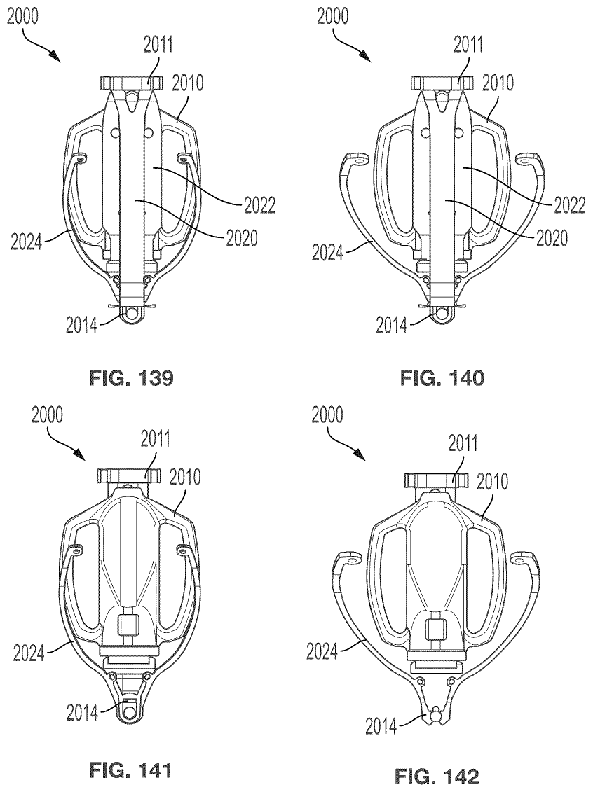

Referring to FIGS. 137-148, an example implementation of a device or implant 2000 (FIGS. 139-144) includes an anchor portion 2006 having one or more paddle frames 2024. The paddle frames 2024 are configured to allow the device 2000 to maneuver more easily into position for implantation in the heart by reducing the contact and/or friction between the native structures of the heart—e.g., chordae—and the device 2000. For example, lines are controlled by a user to create a compression force (e.g., compression force C shown in FIG. 128) on the paddle frames 2024 to move the paddle frames 2024 from a normal, expanded position (FIGS. 140 and 142) to a narrowed position (FIGS. 139 and 141) as the device 2000 is being positioned for implantation on the leaflets of a native valve such that the contact between the native structures of the heart and the device 2000 is reduced. The device 2000 can include any other features of devices or implants discussed elsewhere in the present application or in the applications and patents incorporated by reference herein, and the device 2000 can be positioned to engage valve tissue 20, 22 as part of any suitable valve repair system (e.g., any valve repair system disclosed in the present application). In addition, any of the devices described herein can incorporate the features of the device 2000.

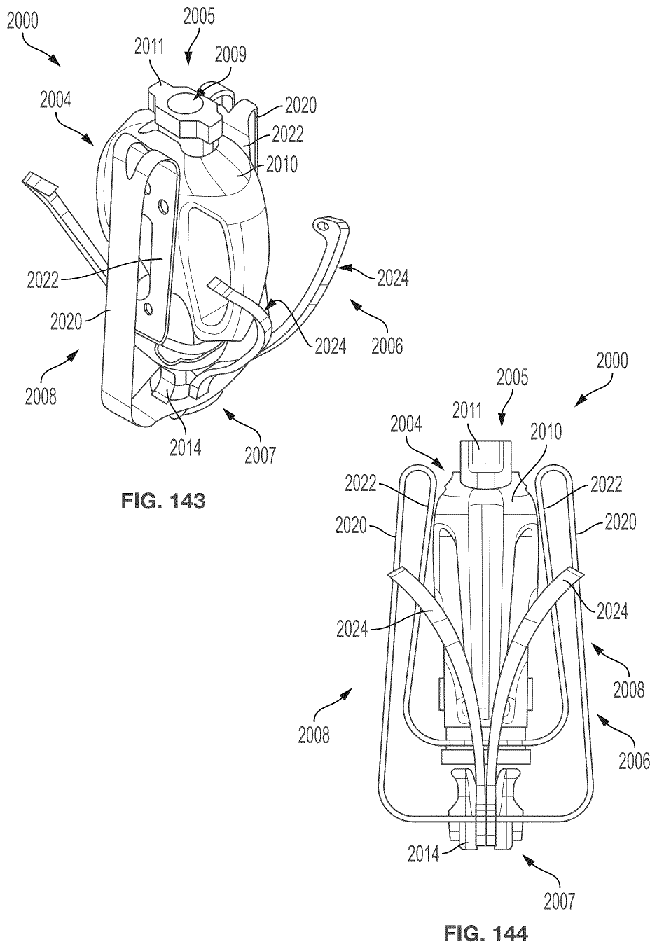

Referring to FIGS. 143-144, the device or implant 2000 includes a coaptation portion 2004, a proximal or attachment portion 2005, an anchor portion 2006, and a distal portion 2007. The coaptation portion 2004, attachment portion 2005, and distal portion 2007 can take any suitable form, such as, for example, the form for these portions of the device 200 shown in FIGS. 22-37, or any other form described in the present application. In some implementations, the coaptation portion 2004 optionally includes a coaptation element 2010 (e.g., a spacer, coaption element, gap filler, etc.) that can be used, for example, for implantation between the leaflets 20, 22 of the native mitral valve MV. The coaptation element 2010 can take any suitable form, such as, for example, any form described in the present application.

The attachment portion 2005 includes a first or proximal collar 2011 for engaging with a capture mechanism (e.g., the capture mechanism 213 shown in FIGS. 44-49) of a delivery system (e.g., the delivery system 202 shown in FIGS. 38-49). The proximal collar 2011 can take any suitable form, such as, for example, any form described in the present application.

The distal portion 2007 includes a cap 2014 that is attached to anchors 2008 of the anchor portion 2006 such that movement of the cap 2014 causes the anchors 2008 to move between open and closed positions. The cap 2014 can take any suitable form, such as, for example, any form described in the present application. An actuation element (e.g., the same as or similar to actuation element 212 shown in FIGS. 22-37) extends from a delivery system (e.g., any delivery system described in the present application), through the coaptation element 2010 via opening 2009 (FIG. 143), and engages the cap 2014 to move the cap 2014 relative to the coaptation element 2010 to enable actuations of the device 2000. The actuation element can engage and move the cap by any suitable means, such as, for example, any means provided in the present application.

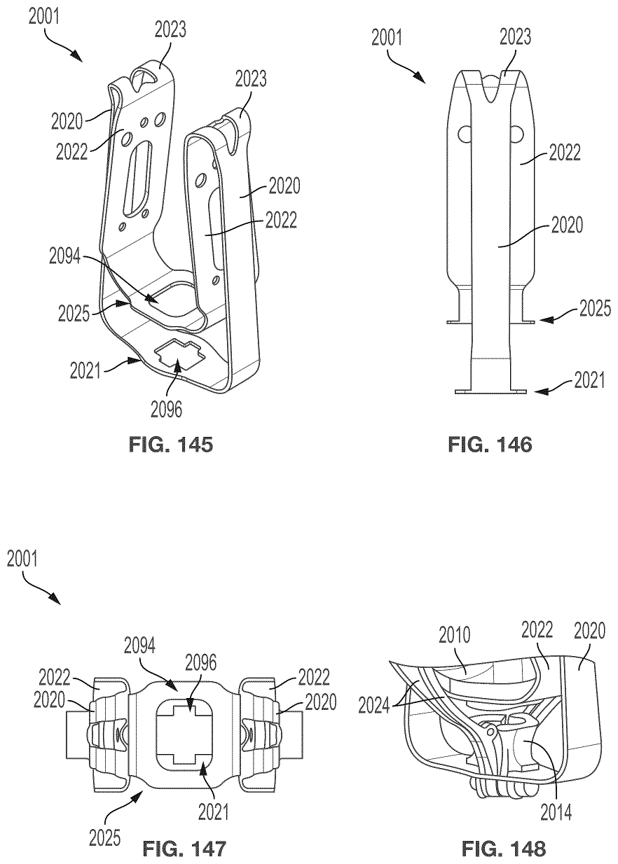

The anchor portion 2006 can take any suitable form, such as, for example, the form of the anchor portion 206 of the device 200 shown in FIGS. 22-37 or any other form described in the present application. The anchor portion 2006 can include a plurality of anchors 2008, each anchor 2008 including outer paddles 2020, inner paddles 2022, paddle extension members or paddle frames 2024, and clasps (e.g., clasps 230 shown in FIGS. 22-37). Referring to FIGS. 137 and 138, the paddle frames 2024 can include a main support section 2085 and connection members 2003 (e.g., projections, cutouts, complementary surfaces, connectors, fasteners, etc.) for attaching to the cap 2014. The paddle frame 2024 can attach to the cap 2014 by any suitable means, such as, for example, any means described in the present application. Referring to FIGS. 145-148, in the illustrated example both of the anchors 2008 include are defined by a paddle ribbon 2001 that includes the inner paddle 2022 and the outer paddle 2020 of each anchor 2008. The inner paddles 2022 of each anchor 2008 are attached by a connection portion 2025 that is configured to connect the inner paddles 2022 to the coaptation element 2010 (as shown in FIG. 148). In the illustrated example, the connection portion 2025 includes an opening 2094 for receiving a distal portion of the coaptation element 2010. The outer paddles 2020 of each anchor 2008 are attached by a connection portion 2021 that is configured to connect the outer paddles 2020 to the cap 214 (as shown in FIG. 148). In the illustrated example, the connection portion 2021 includes an opening 2096 for receiving a portion of the cap 2014. Each inner paddle 2022 is attached to the corresponding outer paddle 2020 by connection portion 2023.

Referring to FIGS. 137 and 138, the paddle frame 2024 includes two or more arms 2080 that define the total width TW of the anchors 2008, in which the at least some of the arms 2080 are connected at a distal portion of the paddle frame 2024 (e.g., a portion of the paddle frame 2024 proximate the connection members 2003). Each of the arms 2080 includes one or more openings 2091, 2092 for receiving one or more lines (e.g., lines 1890 shown in FIGS. 127-130) such that a user can pull on the lines to cause the paddle frame 2024 to move to the narrowed position. The illustrated example includes two arms 2080 that each include a proximal opening 2091 and a distal opening 2092. In some implementations, a single line can extend through each opening 2091, 2092 such that the single line can cause the paddle frame 2024 to move to the narrowed position. It should be understood, however, that any suitable number of lines can extend through the openings 2091, 2092 to cause the paddle frame 2024 to move to the narrowed position.

Referring to FIG. 137, the arms 2080 are connected to each other at the distal portion of the paddle frame 2024 by a connection link 2083. This connection between the two arms 2080 causes the arms 2080 to pivot, flex, and/or articulate about the connection link 2083 in an inward direction Z when a user causes a tensioning force F on the paddle frame 2024 by pulling the one or more lines that extend through the openings 2091, 2092. This pivoting, flexing, and/or articulating of the arms 2080 causes the main support section 2085 of the arms 2080 to move in the inward direction X such that the paddle frame 2024 is in the narrowed position. In the illustrated example, the connection link 2083 has a first member 2087 (e.g., metal portion, leg, etc.) attached to one arm 2080, a second member 2089 (e.g., metal portion, leg, etc.) attached to the other arm 2080, and a thin arched member 2086 (e.g., metal portion, strut, etc.) that connects the first member 2087 to the second member 2089. The connection link 2083 can, however, take any suitable form that allows the arms to pivot, flex, and/or articulate in the inward direction Z when a tensioning force F is applied to the paddle frame 2024. In some implementations, the connection link 2083 is integral to the arms 2080 of the paddle frame 2024.

Still referring to FIGS. 137, the total width TW of the paddle frame 1924 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the normal width to the narrowed width W2 can be between 10/9 and 3/1, such as between 5/4 and 2/1, such as between 4/3 and 3/2.

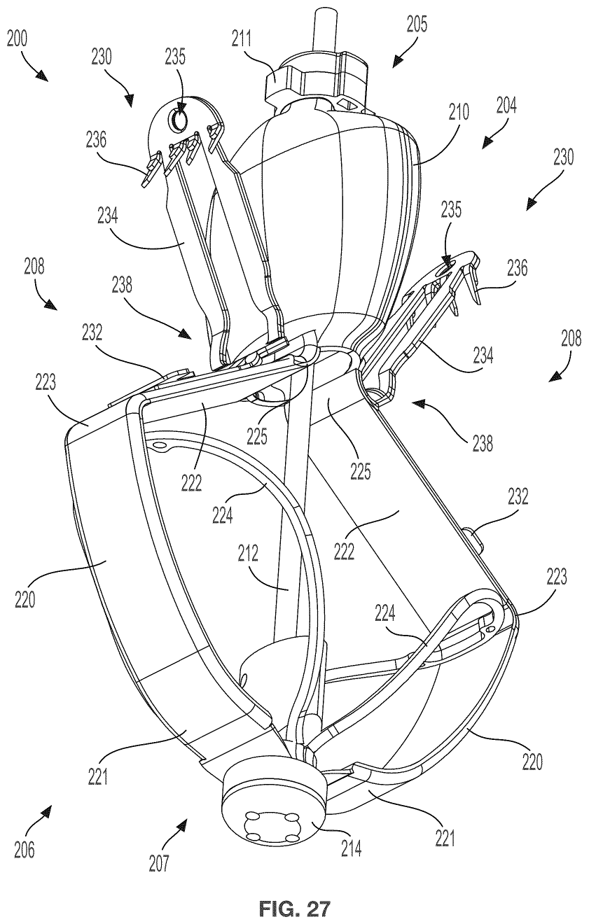

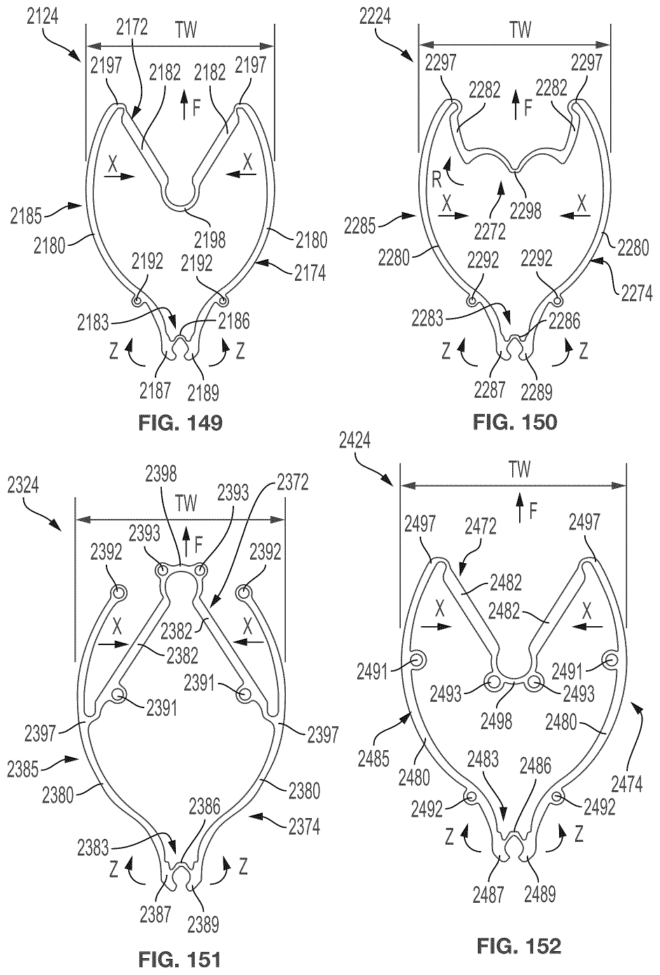

FIGS. 149-156 show some implementations of the paddle frame 2024 that can be used with the device or implant 2000 shown in FIGS. 139-144. Referring to FIG. 149, the device 2000 can include a paddle frame 2124 having an inner frame portion 2172 and an outer frame portion 2174. The outer frame portion 2174 has two arms 2180 that each include an opening 2192 for receiving one or more lines (e.g., line 1890 shown in FIGS. 27-30) such that a user can pull the line to cause the paddle frame to move to the narrowed position. The arms 2180 define the total width TW of the anchors of the device 2000.

The arms 2180 are connected to each other at the distal portion of the paddle frame 2124 by a connection link 2183. This connection between the two arms 2180 causes the arms 2180 to pivot, flex, and/or articulate about the connection link 2183 in an inward direction Z when a user causes a tensioning force F on the paddle frame 2024 by pulling the one or more lines that extend through the openings 2192. This pivoting, flexing, and/or articulating of the arms 2180 causes the main support section 2185 of the arms 2180 to move in the inward direction X such that the paddle frame 2124 is in the narrowed position. In the illustrated example, the connection link 2183 has a first member 2187 (e.g., metal portion, leg, etc.) attached to one arm 2180, a second member 2189 (e.g., metal portion, leg, etc.) attached to the other arm 2180, and a thin arched member 2186 (e.g., metal portion, strut, etc.) that connects the first member 2187 to the second member 2189. The connection link 2183 can take any suitable form, such as, for example, any form described for the connection link 2083 shown in FIG. 137.

The inner frame portion 2172 of the paddle frame 2124 has two arms 2182 that extend inward and downward from the proximal portion of the arms 2180 and help facilitate movement of the paddle frame 2124 to the narrowed position. The arms 2182 are connected to the arms 2180 at connection points 2197. In some implementations, the connection points 2197 include a thin arched portion that helps facilitate movement of the arms 2180, 2182 in the inward direction X. The arms 2182 are connected to each other at connection point 2198. The connection point 2198 can include a thin rounded portion that further helps facilitate movement of the arms 2180, 2182 in the inward direction X.

Referring to FIG. 150, in some implementations, the device 2000 can include a paddle frame 2224 having an inner frame portion 2272 and an outer frame portion 2274. The outer frame portion 2274 has two arms 2280 that each include an opening 2292 for receiving one or more lines (e.g., line 1890 shown in FIGS. 27-30) such that a user can pull the line to cause the paddle frame to move to the narrowed position. The arms 2280 define the total width TW of the anchors of the device 2000.

The arms 2280 are connected to each other at the distal portion of the paddle frame 2224 by a connection link 2283. This connection between the two arms 2280 causes the arms 2280 to pivot, flex, and/or articulate about the connection link 2283 in an inward direction Z when a user causes a tensioning force F on the paddle frame 2224 by pulling the one or more lines that extend through the openings 2292. This pivoting, flexing, and/or articulating of the arms 2280 causes the main support section 2285 of the arms 2280 to move in the inward direction X such that the paddle frame 2224 is in the narrowed position. In the illustrated example, the connection link 2283 has a first member 2287 (e.g., metal portion, leg, etc.) attached to one arm 2280, a second member 2289 (e.g., metal portion, leg, etc.) attached to the other arm 2280, and a thin arched member 2286 (e.g., metal portion, strut, etc.) that connects the first member 2287 to the second member 2289. The connection link 2283 can take any suitable form, such as, for example, any form described for the connection link 2083 shown in FIG. 137.

The inner frame portion 2272 of the paddle frame 224 has two arms 2282 that extend inward and downward from the proximal portion of the arms 2280 and help facilitate movement of the paddle frame 2224 to the narrowed position. The arms 2282 are connected to the arms 2280 at connection points 2297. In some implementations, the connection points 2297 include a thin arched portion that helps facilitate movement of the arms 2280, 2282 in the inward direction X. The arms 2282 are connected to each other at connection point 2298. The connection point 2298 can include a thin arched portion that further helps facilitate movement of the arms 2280, 2282 in the inward direction X. In the illustrated example, each of the arms 2282 have a concave portion that attach to each other at the thin arched portion of the connection point 2298 to help facilitate flexing of the arms 2282 in the inward direction X.

Referring to FIG. 151, in some implementations, the device 2000 can include a paddle frame 2324 having an inner frame portion 2372 and an outer frame portion 2374. The outer frame portion 2374 has two arms 2380 that each include an opening 2392 for receiving one or more lines (e.g., line 1890 shown in FIGS. 27-30) such that a user can pull the line to cause the paddle frame to move to the narrowed position. The arms 2380 define the total width TW of the anchors of the device 2000.

The arms 2380 are connected to each other at a proximal portion of the paddle frame 2324 by a connection link 2383. This connection between the two arms 2380 causes the arms 2380 to pivot, flex, and/or articulate about the connection link 2383 in an inward direction Z when a user causes a tensioning force F on the paddle frame 2324 by pulling the one or more lines that extend through the openings 2392. This pivoting, flexing, and/or articulating of the arms 2380 causes the main support section 2385 of the arms 2280 to move in the inward direction X such that the paddle frame 2324 is in the narrowed position. In the illustrated example, the connection link 2283 has a first member 2387 (e.g., metal portion, leg, etc.) attached to one arm 2380, a second member 2389 (e.g., metal portion, leg, etc.) attached to the other arm 2380, and a thin arched member 2386 (e.g., metal portion, strut, etc.) that connects the first member 2387 to the second member 2389. The connection link 2383 can take any suitable form, such as, for example, any form described for the connection link 2083 shown in FIG. 137.

The inner frame portion 2372 of the paddle frame 2324 has two arms 2382 that extend inward and upward from the arms 2380 and help facilitate movement of the paddle frame 2324 to the narrowed position. The arms 2382 are connected to the arms 2380 at connection points 2397. In some implementations, the connection points 2397 include a thin arched portion that helps facilitate movement of the arms 2380, 2382 in the inward direction X. The arms 2282 are connected to each other at connection point 2398. The connection point 2398 can include a thin rounded portion that further helps facilitate movement of the arms 2380, 2382 in the inward direction X. The arms 2382 each include a distal opening 2391 and a proximal opening 2393 that can receive the one or more lines. In some implementations, the openings 2393 can be connected to the connection portion 2023 (FIG. 145) of the anchors 2008 such that movement of the anchors to the open position provides a further tension force F on the paddle frame 2324 to facilitate movement of the paddle frame 2324 to the narrowed position.

Referring to FIG. 152, the device 2000 can include a paddle frame 2424 having an inner frame portion 2472 and an outer frame portion 2474. The outer frame portion 2474 has two arms 2480 that each include a distal opening 2492 and a proximal opening 2491 for receiving one or more lines (e.g., line 1890 shown in FIGS. 27-30) such that a user can pull the line to cause the paddle frame to move to the narrowed position. The arms 2480 define the total width TW of the anchors of the device 2000.

The arms 2480 are connected to each other at the distal portion of the paddle frame 2424 by a connection link 2483. This connection between the two arms 2480 causes the arms 2480 to pivot, flex, and/or articulate about the connection link 2483 in an inward direction Z when a user causes a tensioning force F on the paddle frame 2424 by pulling the one or more lines that extend through the openings 2492. This pivoting, flexing, and/or articulating of the arms 2480 causes the main support section 2485 of the arms 2480 to move in the inward direction X such that the paddle frame 2424 is in the narrowed position. In the illustrated example, the connection link 2483 has a first member 2487 (e.g., metal portion, leg, etc.) attached to one arm 2480, a second member 2489 (e.g., metal portion, leg, etc.) attached to the other arm 2480, and a thin arched member 2486 (e.g., metal portion, strut, etc.) that connects the first member 2487 to the second member 2489. The connection link 2483 can take any suitable form, such as, for example, any form described for the connection link 2083 shown in FIG. 137.

The inner frame portion 2472 of the paddle frame 2424 has two arms 2482 that extend inward and downward from the proximal portion of the arms 2480 and help facilitate movement of the paddle frame 2424 to the narrowed position. The arms 2482 are connected to the arms 2480 at connection points 2497. In some implementations, the connection points 2497 include a thin arched portion that helps facilitate movement of the arms 2480, 2482 in the inward direction X. The arms 2482 are connected to each other at connection point 2498. The connection point 2498 can include a thin rounded portion that further helps facilitate movement of the arms 2480, 2482 in the inward direction X. The arms 2482 each include an opening 2493 that can receive the one or more lines.

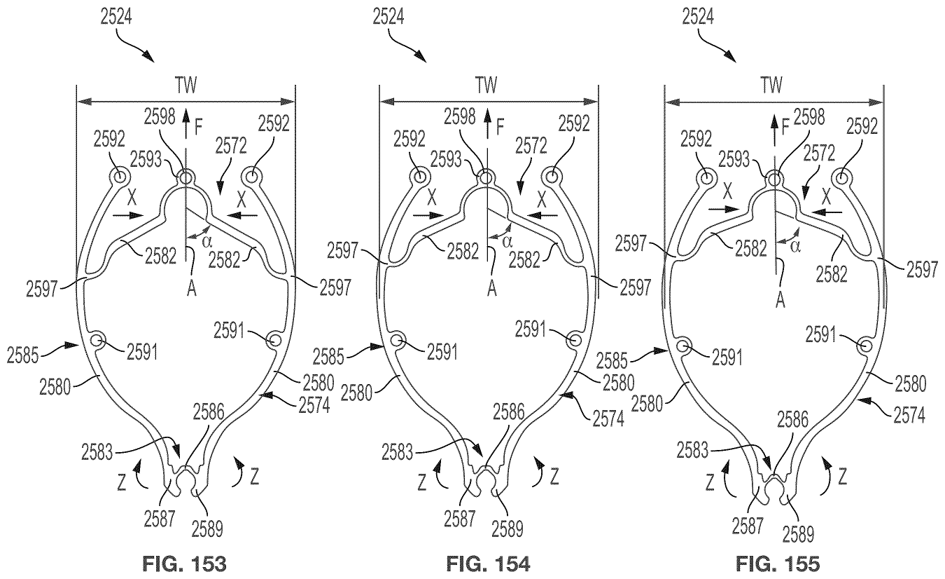

Referring to FIGS. 153-155, in some implementations, the device 2000 can include a paddle frame 2524 having an inner frame portion 2572 and an outer frame portion 2574. The outer frame portion 2574 has two arms 2580 that each include a proximal opening 2592 and a distal opening 2591 for receiving one or more lines (e.g., line 1890 shown in FIGS. 27-30) such that a user can pull the line to cause the paddle frame to move to the narrowed position. The arms 2580 define the total width TW of the anchors of the device 2000.

The arms 2580 are connected to each other at a proximal portion of the paddle frame 2524 by a connection link 2583. This connection between the two arms 2580 causes the arms 2580 to pivot, flex, and/or articulate about the connection link 2583 in an inward direction Z when a user causes a tensioning force F on the paddle frame 2524 by pulling the one or more lines that extend through the openings 2591, 2592. This pivoting, flexing, and/or articulating of the arms 2580 causes the main support section 2585 of the arms 2580 to move in the inward direction X such that the paddle frame 2524 is in the narrowed position. In the illustrated example, the connection link 2583 has a first member 2587 (e.g., metal portion, leg, etc.) attached to one arm 2580, a second member 2589 (e.g., metal portion, leg, etc.) attached to the other arm 2580, and a thin arched member 2586 (e.g., metal portion, strut, etc.) that connects the first member 2587 to the second member 2589. The connection link 2583 can take any suitable form, such as, for example, any form described for the connection link 2083 shown in FIG. 137.

The inner frame portion 2572 of the paddle frame 2524 has two arms 2582 that extend inward and upward from the arms 2580 and help facilitate movement of the paddle frame 2524 to the narrowed position. The arms 2582 are connected to the arms 2580 at connection points 2597. In some implementations, the connection points 2597 include a thin arched portion that helps facilitate movement of the arms 2580, 2582 in the inward direction X. The arms 2582 are connected to each other at connection point 2598. The connection point 2598 can include a thin rounded portion that further helps facilitate movement of the arms 2580, 2582 in the inward direction X. The connection point 2598 includes an opening 2593 that can receive the one or more lines. In some implementations, the opening 2593 can be connected to the connection portion 2023 (FIG. 145) of the anchors 2008 such that movement of the anchors to the open position provides a further tension force F on the paddle frame 2524 to facilitate movement of the paddle frame 2524 to the narrowed position.

In some implementations, an angle α exists between each arm 2582 of the inner frame portion 2372 of the paddle frame 2524 and an axis A that bisects the paddle frame 2524. Referring to FIG. 153, the angle α can be about 60 degrees. Referring to FIG. 154, the angle α can be about 65 degrees. Referring to FIG. 155, the angle α can be about 70 degrees. While the angle is shown as being 60, 65, or 70 degrees, it should be understood that the angle α can take any suitable form that allows the paddle frame 2524 to move to the narrowed position when the force F is applied to the paddle frame, such as between 50 degrees and about 80 degrees.

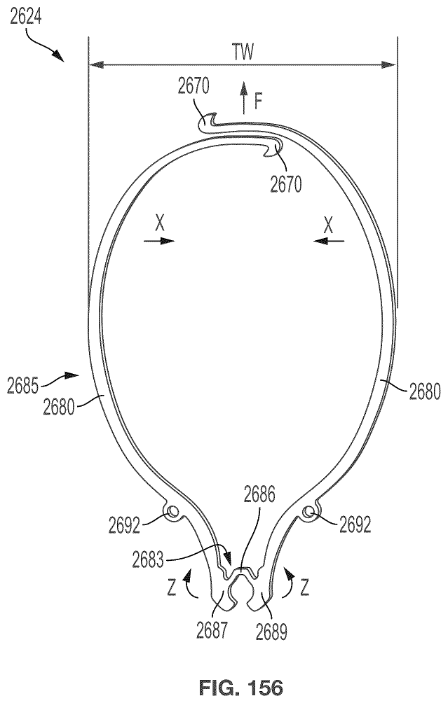

Referring to FIG. 156, the device 2000 can include a paddle frame 2624 with two arms 2680 that each include an opening 2692 for receiving one or more lines (e.g., line 1890 shown in FIGS. 27-30) such that a user can pull the line to cause the paddle frame to move to the narrowed position. The arms 2680 define the total width TW of the anchors of the device 2000. The proximal portions 2670 of the arms 2680 are offset from each other such that one of the arms 2680 can extend over the other arm 2680 when the arms 2680 move in the inward direction X, and the proximal portions 2670 of the arms 2680 can move away from each other to move back to the normal, expanded positions.

The arms 2680 are connected to each other at the distal portion of the paddle frame 2124 by a connection link 2683. This connection between the two arms 2680 causes the arms 2680 to pivot, flex, and/or articulate about the connection link 2683 in an inward direction Z when a user causes a tensioning force F on the paddle frame 2624 by pulling the one or more lines that extend through the openings 2692. This pivoting, flexing, and/or articulating of the arms 2680 causes the main support section 2685 of the arms 2680 to move in the inward direction X such that the paddle frame 2624 is in the narrowed position. In the illustrated example, the connection link 2683 has a first member 2687 (e.g., metal portion, leg, etc.) attached to one arm 2680, a second member 2689 (e.g., metal portion, leg, etc.) attached to the other arm 2680, and a thin arched member 2686 (e.g., metal portion, strut, etc.) that connects the first member 2687 to the second member 2689. The connection link 2683 can take any suitable form, such as, for example, any form described for the connection link 2083 shown in FIG. 137.

Referring FIGS. 149-156, the total width TW of the paddle frame 2024-2524 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the normal width TW to the narrowed width can be between 10/9 and 3/1, such as between 5/4 and 2/1, such as between 4/3 and 3/2.

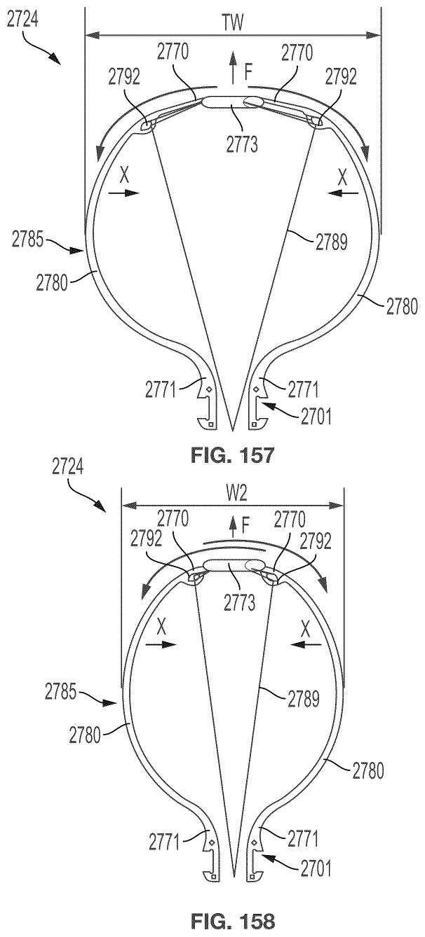

Referring to FIGS. 157 and 158, an example of a paddle frame 2724 for a device or implant (e.g., device 200 shown in FIGS. 22-37, device 600 shown in FIG. 94, or any other suitable device described in the present application) includes a main support section 2785 and connection members 2701 (e.g., cutout, protrusion, complementary surface, connector, fastener, etc.) for attaching to a cap of the device or implant. The paddle frame 2724 is configured to allow the device to maneuver more easily into position for implantation in the heart by reducing the contact and/or friction between the native structures of the heart—e.g., chordae and the device. For example, lines (e.g., lines 1890 shown in FIGS. 127-130) are controlled by a user to create a compression force on the paddle frames 2724 to move the paddle frames 2724 from a normal, expanded position (FIG. 157) to a narrowed position (FIG. 158) as the device is being positioned for implantation on the leaflets of a native valve such that the contact between the native structures of the heart and the device is reduced.

The connection members 2701 can take any suitable form, such as, for example, any form described in the present application. The paddle frame 2724 can attach to the cap by any suitable means, such as, for example, any means described in the present application. The thickness and width of the paddle frame 2724 can take any suitable form, such as, for example, the thickness can be substantially identical to the width, the thickness can be greater than the width (as shown in FIGS. 91-95), or the width can be greater than the thickness.

The paddle frame 2724 includes arms 2780 that each extend from a distal portion 2771 to a proximal portion 2770. The arms 2780 define the total width TW of the anchors of the device or implant. The proximal portions 2770 of the arms 2780 include openings 2792 for receiving a width adjustment line 2789 that is connected to the device or implant (e.g., connected to the cap of the device) such that the width adjustment line 2789 controls the total width of the paddle frame 2724.

The proximal portions 2770 of the arms 2780 are loosely connected by a sleeve member 2773 (tube, sleeve, straw, conduit, etc.) that allows the proximal portions 2770 of the arms 2780 to move relative to each other. That is, the proximal portions 2770 can be offset from each other (e.g., similar to the paddle frame 2624 shown in FIG. 156) such that one of the arms 2780 can extend over the other arm 2780 when the arms 2780 move in the inward direction X, and the proximal portions 2770 of the arms 2680 can move away from each other to move back to the normal, expanded position. When the paddle frame 2724 is in the narrowed position W2, at least a portion of the proximal portions 2770 of the arms 2780 are disposed within the sleeve 2773.

A user can move the paddle frame between the normal, expanded position (FIG. 157) and the narrowed position (FIG. 158) by pulling a line to create a tension force F on the paddle frame 2724. The line can be connected to the paddle frame (e.g., to the openings 2792) or to the width adjustment line 2789 such that a pulling force to the line causes the tension force F on the paddle frame 2724. Referring to FIG. 158, when the tension force F is applied to the paddle frame, the proximal portions 2770 of the arms 2780 move toward each other in the direction X such that the paddle frame 2724 moves to the narrowed position. The movement of the paddle frame 2724 to the narrowed position allows the device or implant to more easily maneuver into position for implantation in the heart by reducing the contact and/or friction between the native structures of the heart—e.g., chordae—and the device.

The total width TW of the paddle frame 2724 when in the normal, expanded position can be between 5 mm and 15 mm, such as between 7 mm and 12 mm, such as between 9 mm and 11 mm, such as about 10 mm. The narrowed width W2 of the paddle frame 1124 can be between 3 mm and 12 mm, such as between 5 mm and 10 mm, such as between 7 mm and 9 mm, such as about 8 mm. A ratio of the normal width to the narrowed width W2 can be between 10/9 and 3/1, such as between 5/4 and 2/1, such as between 4/3 and 3/2.

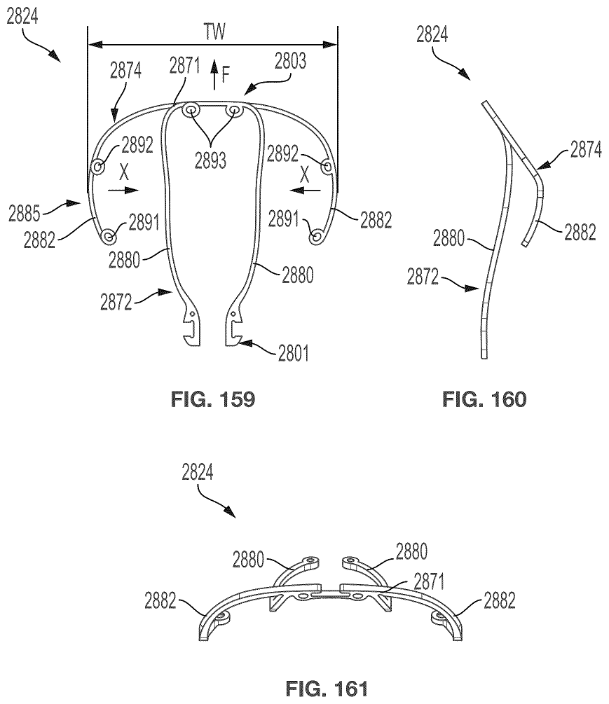

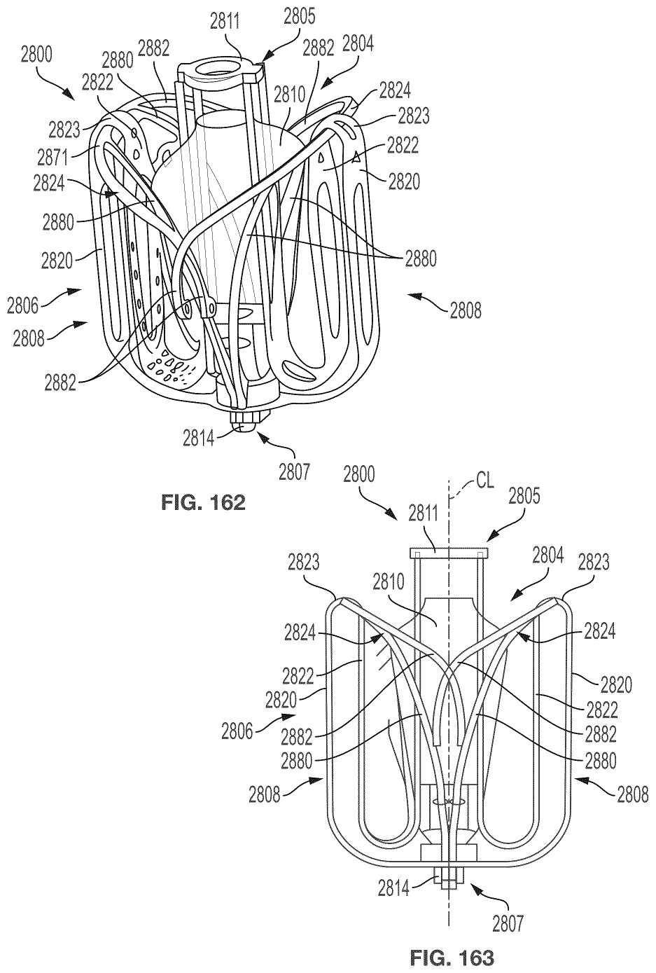

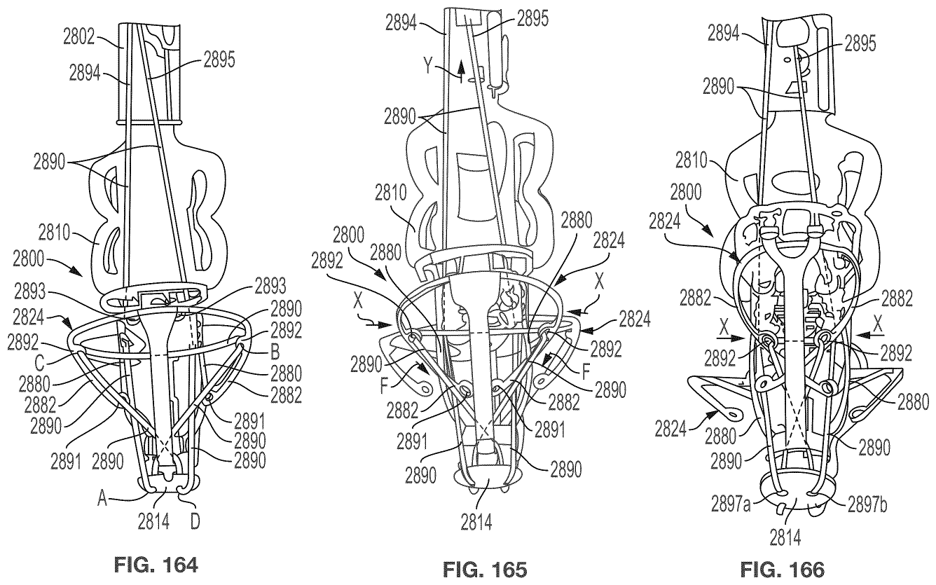

Referring to FIGS. 159-168, an example implementation of a device or implant 2800 (FIGS. 162-168) includes an anchor portion 2806 having one or more paddle frames 2824 that are movable to a narrowed position to allow the device 2800 to maneuver more easily into position for implantation in the heart by reducing the contact and/or friction between the native structures of the heart—e.g., chordae—and the device 2800. That is, one or more width adjustment elements (e.g., line, suture, wire, metal component, etc.), such as the illustrated lines 2890 (FIGS. 164-168) are controlled by a user to create a compression force on the paddle frames 2824 to move the paddle frames 2824 to a narrowed position as the device 2800 is being positioned for implantation on the leaflets of a native valve such that the contact between the native structures of the heart and the device 1800 is reduced. The device 2800 can include any other features of devices or implants discussed elsewhere in the present application or in the applications and patents incorporated by reference herein, and the device 2800 can be positioned to engage valve tissue 20, 22 as part of any suitable valve repair system (e.g., any valve repair system disclosed in the present application). In addition, any of the devices described herein can incorporate the features of the device 2800.

Referring to FIGS. 162-163, the device or implant 2800 includes a coaptation portion 2804, a proximal or attachment portion 2805, an anchor portion 2806, and a distal portion 2807. The coaptation portion 2804, attachment portion 2805, and distal portion 2807 can take any suitable form, such as, for example, the form for these portions of the device 200 shown in FIGS. 22-37, or any other form described in the present application. In some implementations, the coaptation portion 2804 optionally includes a coaptation element 2810 (e.g., a spacer, coaption element, gap filler, etc.) that can be used, for example, for implantation between the leaflets 20, 22 of the native mitral valve MV. The coaptation element 2810 can take any suitable form, such as, for example, any form described in the present application.

The attachment portion 2805 includes a first or proximal collar 2811 for engaging with a capture mechanism (e.g., capture mechanism 213 shown in FIGS. 44-49) of a delivery s system 2802. The capture mechanism and the delivery system 2802 can take any suitable form, such as, for example, any form described in the present application. The delivery system 2802 can be the same as or similar to other delivery systems herein, e.g., 102, 202, 402, 502, etc. and can comprise one or more of a catheter, a sheath, a guide catheter/sheath, a delivery catheter/sheath, a steerable catheter, an implant catheter, a tube, a channel, a pathway, combinations of these, etc. The proximal collar 2811 can take any suitable form, such as, for example, any form described in the present application.

The distal portion 2807 includes a cap 2814 that is attached to anchors 2808 of the anchor portion 1806 such that movement of the cap 2814 causes the anchors 2808 to move between open and closed positions. The cap 2814 can take any suitable form, such as, for example, any form described in the present application. In the illustrated example, an actuation element (e.g., the same as or similar to actuation element 212 shown in FIGS. 22-37) extends from a delivery system (e.g., any delivery system described in the present application) and engages the cap 2814 to move the cap 2814 relative to the coaptation element 2810 to enable actuations of the device 2800. The actuation element can engage and move the cap by any suitable means, such as, for example, any means provided in the present application.

The anchor portion 2806 can take any suitable form, such as, for example, the form of the anchor portion 206 of the device 200 shown in FIGS. 22-37 or any other form described in the present application. The anchor portion 2806 can include a plurality of anchors 2808, each anchor 2808 including outer paddles 2820, inner paddles 2822, paddle extension members or paddle frames 2824, and clasps (e.g., clasps 230 shown in FIGS. 22-37). Referring to FIGS. 159-161, the paddle frames 2824 can include a main support section 2885, first connection members 2801 for attaching to the cap 1814, and second connection members 2803 for attaching to a connection portion 2823 of the anchors 2808. The paddle frame 2824 can attach to the connection portion of the anchors and the cap by any suitable means, such as, for example, any means described in the present application. The thickness and width of the paddle frame 2824 can take any suitable form, such as, for example, the thickness can be substantially identical to the width, the thickness can be greater than the width (as shown in FIGS. 91-95), or the width can be greater than the thickness.

The paddle frame 2824 includes an inner frame portion 2872 and an outer frame portion 2874. The inner frame portion 2872 has arms 2880 that extend from the connection members 2801 to a proximal portion of the paddle frame 2824. The outer frame portion 2874 includes arms 2882 that are connected to arms 2880 at connection point 2871 and extend outward from arms 2880. The arms 2882 define a total width TW of the anchors 2808. The arms 2882 can have one or more openings for receiving one or more lines 2890 such that the lines 2890 can be engaged by a user to move the paddle frame 2824 to the narrowed position by moving the arms 2882 in the inward direction X. In the illustrated example, each of the arms 2882 have a first opening 2892 and a second opening 2891 that is positioned distally from the first opening 2892. The inner frame portion 2872 can include one or more openings 2893 that can be used for connecting to the connection portion 2823 of the anchors 2808 and/or for receiving one or more lines 2890.

Referring to FIGS. 160-164, the arms 2882 of the outer frame portion 2874 can be biased in the direction X (FIGS. 160-161) such that the arms 2882 are configured to extend beyond a center line CL (FIG. 163) of the device 2800 when the anchors 2808 are in the closed position. Referring to FIGS. 162-163, for illustrative purposes, the arms 2882 of the paddle frames 2824 are shown crossing each other to show that the arms 2882 are configured to extend beyond the center line CL of the device 2800. It should be understood, however, that the arms 2882 can be positioned to engage the arms 2882 of the other paddle frame 2824 (rather than cross each other) to create a pinching force between the two anchors 2808. In these examples, when the anchors 2808 have captured the leaflets 20, 22 of the mitral valve MV the biased arms 2882 of each paddle frame 2824 pinch the leaflet tissue between them to better secure the device 2800 to the mitral valve MV.

Referring to FIG. 161, the paddle frame 2824 can have a rounded shape that corresponds to the shape of the coaptation element 2810 such that the anchors 2808 conform around the coaptation element to better secure the leaflet tissue between the anchors 2808 and the coaptation element 2810. The paddle frames 2824 can be formed by shape setting a material such that the arms 2882 are biased away from the arms 2880. For example, the paddle frames 2824 can be made of metals, such as steel, nitinol, etc., plastics, etc.

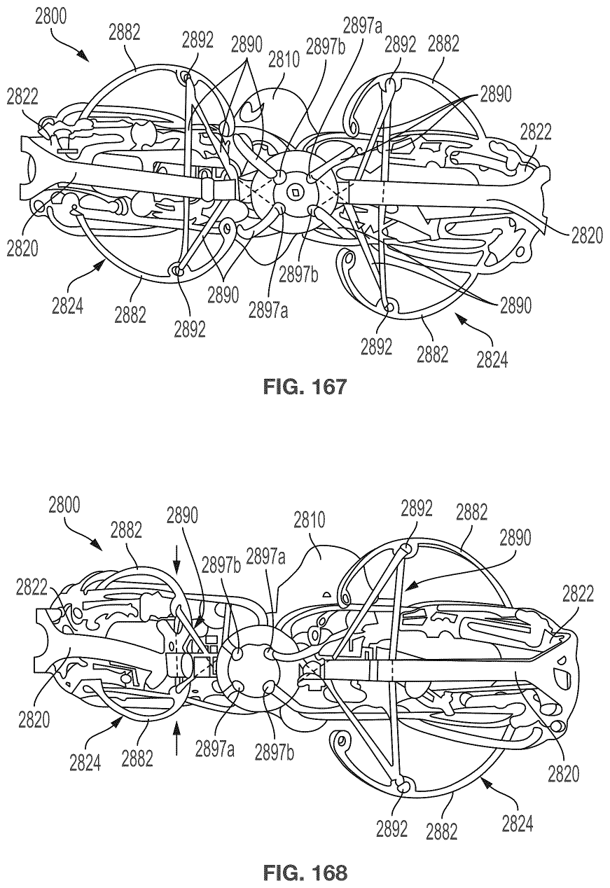

Referring to FIGS. 164-168, in some implementations, each paddle frame 2824 has a corresponding line 2890 that is used to move the paddle frame 2824 from the normal, expanded position (FIGS. 164 and 167) to a narrowed position (FIGS. 165-166 and 168). Each line 2890 can include two ends 2894, 2895 that extend from the delivery system 2802 such that a user can engage the ends 2894, 2895 to cause the paddle frame 2824 to move to the narrowed position. The line 2890 can extend through the cap 2814 before extending through one or more openings (e.g., openings 2891, 2892, 2893) of the paddle frame 2824 and then extending back into the delivery system 2802.

Referring to FIG. 164, in the illustrated example, a first end 2894 of the line 2890 extends from the delivery system 2802 and through an opening 2897a (FIGS. 166-168) of the cap 2814 at point A. Then the line 2890 extends through the opening 2892 of one arm 2882 at point B and then through the opening 2892 of the other arm 2882 at point C. The line 2890 extends back through an opening 2897b (FIGS. 166-168) the cap at point D, and then the second end 2895 of the line 2890 extends back through the delivery system 2802.

Referring to FIG. 165, when a user pulls the ends 2894, 2895, of the line 2890 in the direction Y, the line 2890 causes a tensioning force F on the arms 2882 due to the line extending through the openings 2892. This tensioning force F then causes the arms 2882 to move in the inward direction X such that the paddle frame 2824 is in a narrowed position. Referring to FIG. 166, if the user provides additional force to the ends 2894, 2895 of the line 2890 in the direction Y, the tensioning force F (FIG. 165) continues on the arms 2882 such that the arms 2882 continue to move in the direction X, which can cause the arms 2882 to cross each other (as shown in FIG. 166) such that the paddle frame 2824 is in a more narrowed position. Referring to FIGS. 167 and 168, the paddle frames 2824 can be independently controllable between the normal and narrowed positions. For example, FIG. 167 shows both paddle frames 2824 in the normal position, and FIG. 168 shows one paddle frame 2824 moved to the narrowed position and the other paddle frame 2824 in the normal position.

Referring to FIG. 159, the total width TW of the paddle frame 2824 when in the normal, expanded position can be between 5 mm and 15 mm, such as between 7 mm and 12 mm, such as between 9 mm and 11 mm, such as about 10 mm. The narrowed width of the paddle frame 1124 can be between 3 mm and 12 mm, such as between 5 mm and 10 mm, such as between 7 mm and 9 mm, such as about 8 mm. A ratio of the normal width TW to the narrowed width can be between 10/9 and 3/1, such as between 5/4 and 2/1, such as between 4/3 and 3/2.

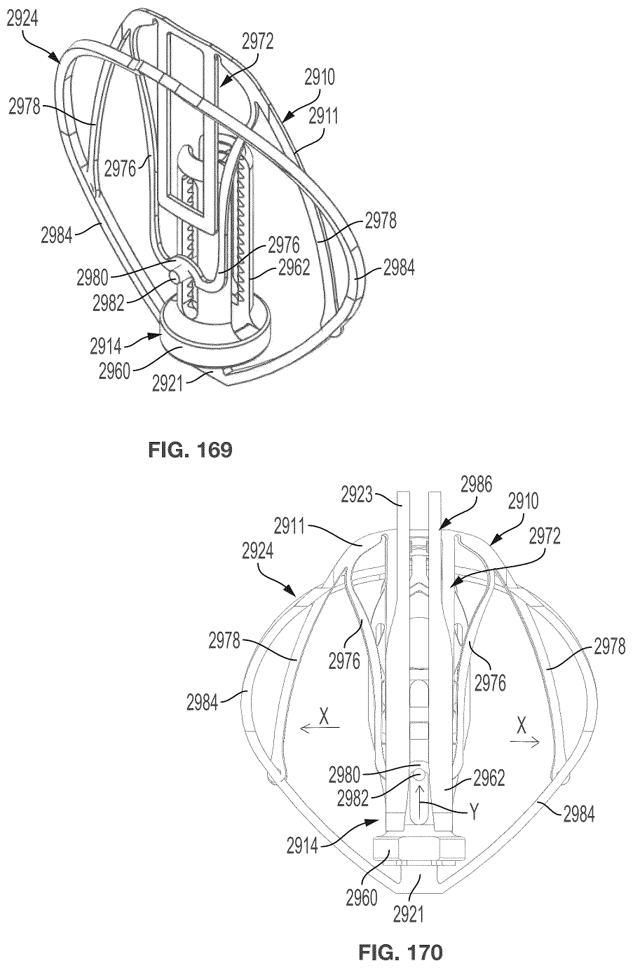

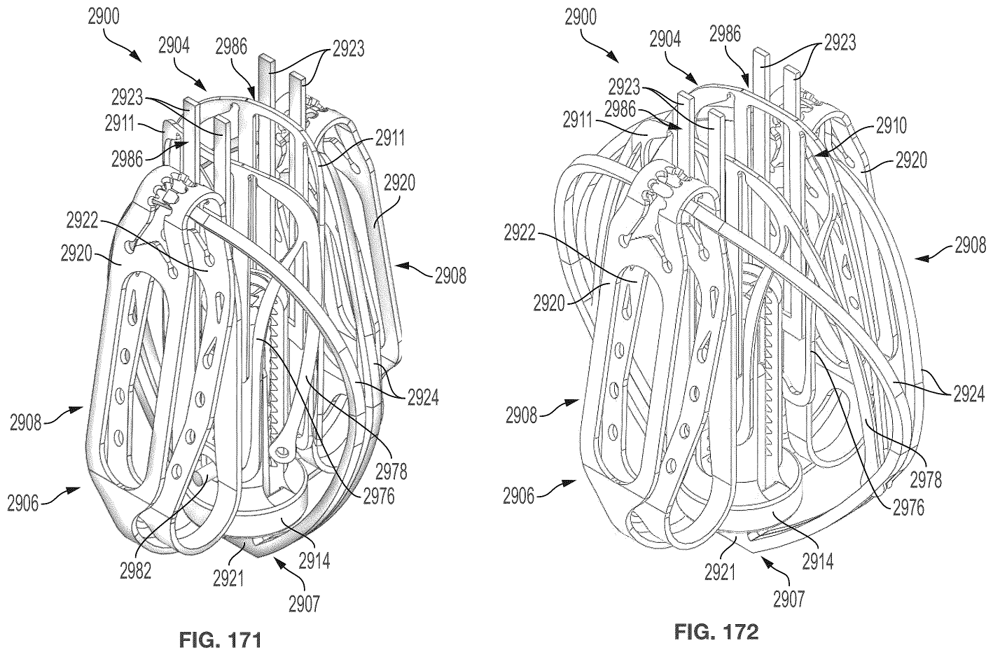

Referring to FIGS. 169-188, an example implementation of a device or implant 2900 (FIGS. 171-172) includes a spacer, a coaptation portion 2904 having a coaptation element 2910 (e.g., a spacer, coaption element, gap filler, etc.) that is movable between a narrowed position (FIG. 171) and an expanded position (FIG. 172). The coaptation element 2910 includes one or more coaptation element frames 2911 that are configured to be expanded such that the coaptation element 2910 has a larger surface area for implantation between the leaflets of a native valve to prevent, inhibit, or reduce regurgitation of blood into the atrium from the ventricle during the systole phase.

The device 2900 can also include an anchor portion 2906 having one or more paddle frames 2924 that are configured to allow the device 2900 to maneuver more easily into position for implantation in the heart by reducing the contact and/or friction between the native structures of the heart—e.g., chordae—and the device 2900. That is, the paddle frames 2924 are configured to move between an expanded position and a narrowed position. When the paddle frames 2924 are in the narrowed position, the contact and/or friction between the native structures of the heart and the device 2900 can be reduced.

The device 2900 can include any other features of devices or implants discussed elsewhere in the present application or in the applications and patents incorporated by reference herein, and the device 2900 can be positioned to engage valve tissue 20, 22 as part of any suitable valve repair system (e.g., any valve repair system disclosed in the present application). In addition, any of the devices described herein can incorporate the features of the device 2900.

Referring to FIGS. 171-172, the device or implant 2900 includes a coaptation portion 2904, a proximal or attachment portion (not shown), an anchor portion 2906, and a distal portion 2907. The attachment portion and distal portion 2907 can take any suitable form, such as, for example, the form for these portions of the device 200 shown in FIGS. 22-37, or any other form described in the present application. The attachment portion can include a first or proximal collar for engaging with a capture mechanism of a delivery sheath or system. The proximal collar, capture mechanism, and delivery system can take any suitable form, such as, for example, any form described in the present application.

The distal portion 2907 includes a cap 2914 that is attached to anchors 2908 of the anchor portion 2906 via an inner end portion 2921 of the paddle frames such that an actuation shaft or wire can be used to engage the cap 2914 and move the anchors 2908 between open and closed positions. The cap 2914 can take any suitable form, such as, for example, any form described in the present application. In the illustrated example, the cap 2914 includes a distal portion 2960 for receiving the inner end portion 2921 of the anchors 2908, a receiver, such as the illustrated threaded portion 2962 fixedly attached to the distal portion, and a threaded member (e.g., threaded shaft, screw, bolt, etc.) disposed within the threaded portion.

The actuation element can engage the threaded member to axially move the entire cap 2914 by providing an axial force to the threaded member. The actuation element can also rotate the threaded member to move the threaded member and, consequently, the inner end portion 2921 of the anchors 2908 relative to the cap 2914. Movement of the entire cap 2914 by providing an axial force to the cap 2914 with an actuation element causes the anchors 2908 to move to the open position.

The anchor portion 2906 of the device can take any suitable form, such as, for example, the form of the anchor portion 206 of the device 200 shown in FIGS. 22-37 or any other form described in the present application. The anchor portion 2906 can include a plurality of anchors 2908, each anchor 2908 including outer paddles 2920, inner paddles 2922, paddle extension members or paddle frames 2924, and clasps (e.g., the clasps 230 shown in FIGS. 22-37). The outer paddles 2920 are jointably attached to the inner paddles 2922 by connection portions. The outer paddles 2920 are attached to an inner end portion 2921 that is positioned within and movable relative to the distal portion 2960 and threaded portion 2962 of the cap 2914. The inner paddles 2922 include a connection portion for connecting to a proximal portion of the coaptation element 2910 (e.g., the proximal portion of the coaptation element frames 2911).

The coaptation portion 2904 includes a coaptation element 2910 that can be used, for example, for implantation between the leaflets 20, 22 of the native mitral valve MV. In the illustrated example, the coaptation element 2910 includes a coaptation element frame 2911 corresponding to each of the anchors 2908 such that the combination of the coaptation element frames 2911 define the outer boundary of the coaptation element 2910.

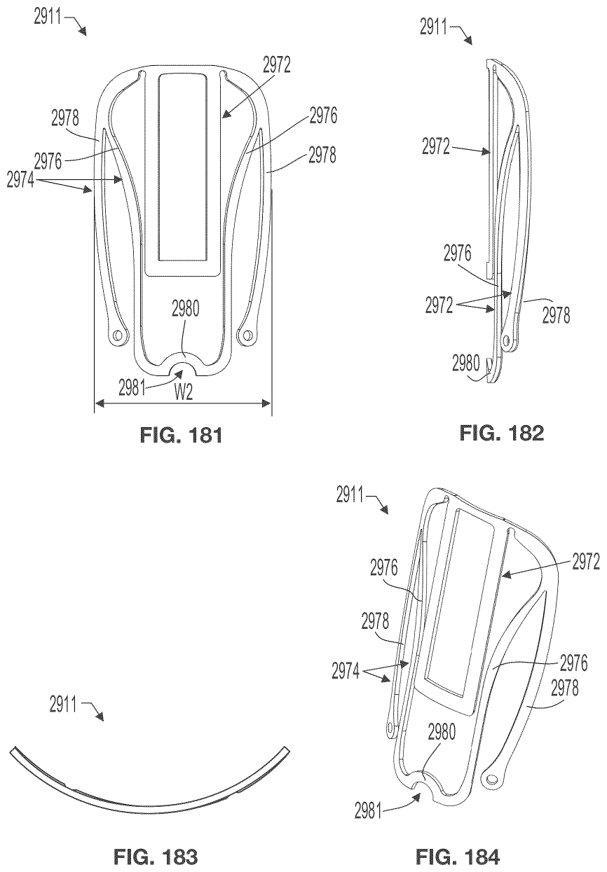

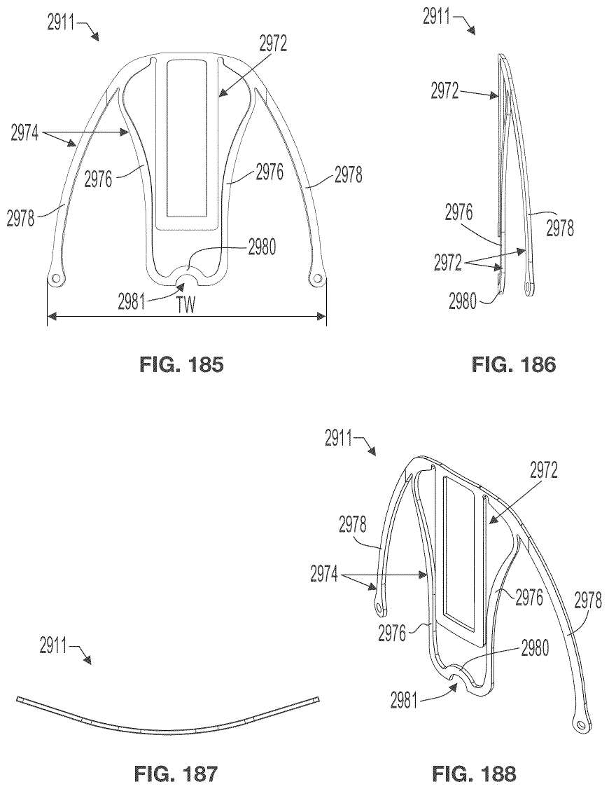

Referring to FIGS. 181-184, an example implementation of the coaptation element frame 2911 is shown in the narrowed position. Referring to FIGS. 185-188, the coaptation element frame 2911 is shown in the expanded position. Referring to FIGS. 181-188, the coaptation element frame 2911 includes a connection portion 2972 for fixedly connecting to the inner paddle 2922 of the anchor 2908. The coaptation element frame 2911 also includes a flexible portion 2974 that includes inner arms 2976 and outer arms 2978. The outer arms 2976 define a total width TW of the coaptation element frame 2911. The inner arms 2976 extend inward and downward from the outer arms 2976 and connect to each other at a connection point 2980. Referring to FIGS. 183 and 187, the coaptation element frame 2911 can have a rounded shape such that the coaptation element 2910 has an elongated rounded shape by a combination of the various coaptation element frames 2911. The combination of the coaptation element frames 2911 can create a coaptation element 2910 that has any suitable shape, such as, for example, the shape of any coaptation element described in the present application.

Referring to FIGS. 169-170, an adjustment member 2982 (e.g., post, shaft, bolt, screw, etc.) is attached to the inner end portion 2921 of the paddle frame 2924, and the adjustment member 2982 is configured to engage the flexible portion 2974 of the coaptation element frame 2911 at connection point 2980 to move the coaptation element frame 2911 to the expanded position. In some examples, the connection point 2980 of the coaptation element frame 2911 includes a notch 2981 for receiving the adjustment member 2982. The notch 2981 can be configured to distribute the force provided by the adjustment member 2982 to the connection point 2980 evenly across the coaptation element frame 2911 such that the force provided by the adjustment member 2982 causes the arms 2978 to expand substantially the same amount in the corresponding directions. For example, in the illustrated example, the notch 2981 is a rounded shape positioned at a central portion of the connection point 2980 between the arms 2976. The notch 2981 can, however, take any other suitable form that causes the force provided by the adjustment member 2982 to be evenly distributed across the coaptation element frame 2911.

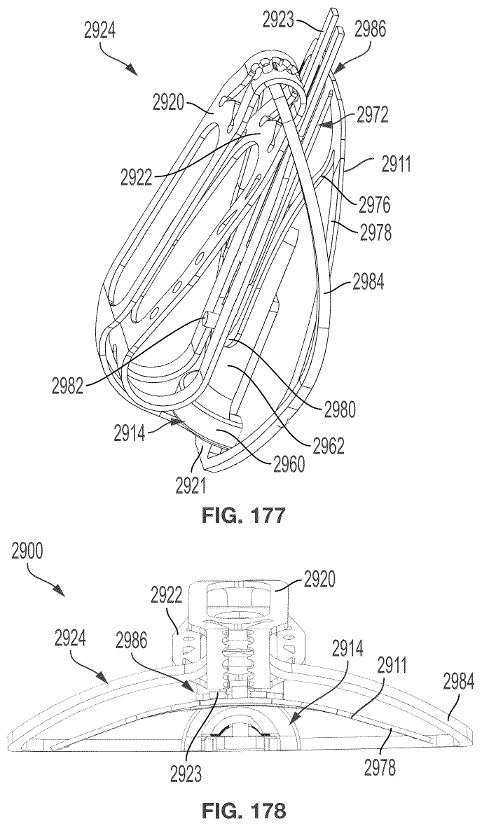

Referring to FIG. 170, movement of the adjustment member 2982 in the direction Y (e.g., by rotating the threaded member within the threaded portion 2962 of the cap 2914 or by removing the axial force to the threaded member from the actuation element such that the anchors 2908 move to the closed position) causes the outer arms 2978 to move in an outward direction X such that the coaptation element frame 2911 is in the expanded position. That is, engagement of the adjustment member 2982 with the connection point 2980 of the coaptation element frame 2911 in the direction Y causes the inner arms 2976 to compress. This compression is due to a connection portion or post 2972 that is fixedly connected to an extension or pair of posts 2923 that extend from the inner paddle 2922. The connection portion or post 2972 and the extension or posts 2923 are connected at connection point 2986. Movement of the pin 2982 pushes the inner arms 2976 up, which causes the outer arms 2978 to move in the outward direction X. The connection between the frame 2911 and the extension or pair of posts 2923 that extends from the inner paddle 2922 at connection point 2986 is shown in FIGS. 177 and 180.

The connection portion 2972 can be connected to the extension 2923 in a wide variety of different ways. For example, the connection portion 2972 and the extension 2923 can be welded, connected with an adhesive, can be integrally formed, and/or can be connected with fasteners. The extension 2923 can take a wide variety of different forms. Any configuration that allows for attachment between the extension 2923 and the connection portion 2972 can be used. The illustrated extension 2923 extends past the connection portion. However, in some implementations, the extension member 2923 need only be long enough to provide a connection between the inner paddles 2922 and the connection portion 2972.

Still referring to FIG. 170, when the adjustment member 2982 is disengaged from the coaptation element frame 2911, the coaptation element frame is maintained in the normal, narrowed position (FIGS. 181-184). The adjustment member 2982 can be disengaged from the coaptation element frame 2911 by, for example, providing an axial force to the cap 2914 with the actuation element such that the entire cap 2914 moves away from the coaptation element 2910, or rotating the threaded member within the threaded portion 2962 of the cap 2914 such that the threaded member engages the inner end portion 2921 to move the inner end portion 2921 and, consequently, the adjustment member 2982 to a disengaged position relative to the coaptation element frame 2911.

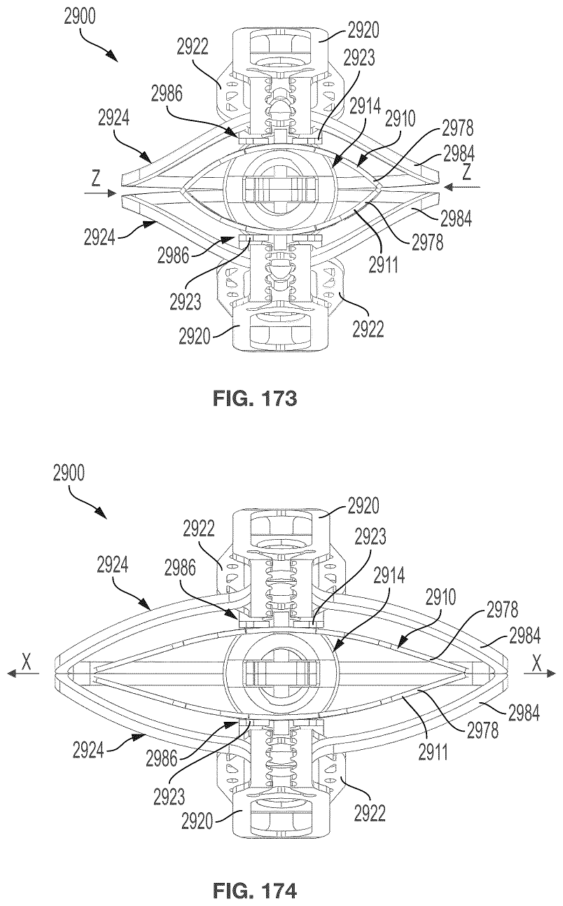

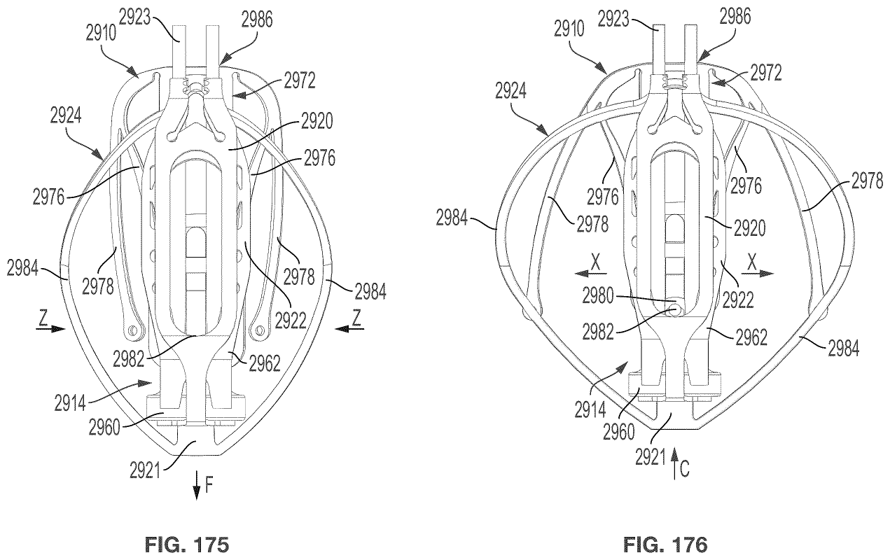

Referring to FIGS. 171-176, the paddle frame 2924 and the coaptation element frame 2911 are movable between an expanded position (FIGS. 172, 174, and 176) and a narrowed position (FIGS. 171, 173, and 175) when the anchors 2908 are in the closed position. For example, referring to FIGS. 173 and 175, movement of the inner end portion 2921 and the adjustment member 2982 (FIG. 176) distally relative to the cap 2914 (by rotating the threaded member within the threaded portion 2962 of the cap 2914 such that the threaded member moves in a distal direction) creates a tension force F (FIG. 175) on the paddle frame 2924 that causes the arms 2984 of the paddle frame 2924 to move in the inward direction Z. In addition, movement of the adjustment member 2982 distally relative to the cap 2914 causes the adjustment member 2982 to disengage the coaptation element frame 2911 and move in the inward direction Z to its normal, narrowed position.

Referring to FIGS. 174 and 176, movement of the inner end portion 2921 and the adjustment member 2982 proximally relative to the cap 2914 (by rotating the threaded member within the threaded portion 2962 of the cap 2914 such that the threaded member moves in a proximal direction) causes a compression force C on the paddle frame 2924 that causes the arms 2984 to move in the outward direction X. In addition, movement of the adjustment member 2982 proximally relative to the cap 2914 causes the adjustment member 2982 to engage the connection point 2980 of the coaptation element frame 2911 and move the arms 2976 in the outward direction X.

Referring to FIGS. 181 and 185, the total width TW of the coaptation element frame 2911 when in the normal, narrowed (FIG. 181) position can be between about be between 4 mm and 8 mm, such as between 5 mm and 7 mm, such as about 6 mm. The total width TW of the coaptation element frame 2911 when in the expanded position (FIG. 185) can be 5 mm and 15 mm, such as between 7 mm and 12 mm, such as between 9 mm and 11 mm, such as about 10 mm. A ratio of a width frame 2911 in the expanded position to the width of the frame 2911 in the narrowed position can be between 10/9 and 3/1, such as between 5/4 and 2/1, such as between 4/3 and 3/2.

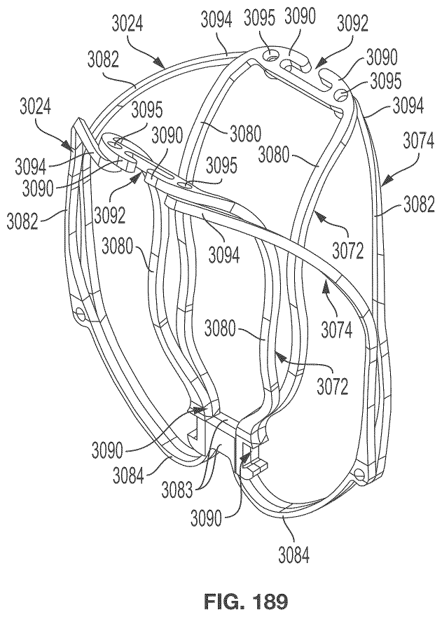

FIGS. 189-192 illustrate an example implementation of paddle frames 3024 for a device or implant, such as any of the device or implants disclosed herein. The paddle frames 3024 are configured to allow the device to maneuver more easily into position for implantation in the heart by reducing the contact and/or friction between the native structures of the heart—e.g., chordae—and the device.

Referring to FIGS. 189-192, each of the paddle frames 3024 can include an inner frame portion 3072 and an outer frame portion 3074. The inner frame portion 3072 includes one or more arms 3080 having a proximal end 3090 and a distal end 3091. The proximal ends 3090 can be connected and have an opening 3092 for receiving the paddles (e.g., the inner and outer paddles) of the anchors 3008. The distal end 3091 can include connection members for attaching to the cap 3014 (FIG. 95) of the distal portion 3007. The illustrated example shows that the inner frame portion 3072 have two arms 3080, but it should be understood that the inner frame portion 3072 can have any suitable number of arms.

The outer frame portion 3074 of each of the paddle frames 3024 has a pair of arms 3082 having a proximal end 3093 and a distal end 3094. The proximal ends 3093 can be configured to attach to the proximal ends 3090 of the inner frame portion 3072. For example, the proximal ends 3090, 3093 of both the inner and outer frame portions 3072, 3074 can include openings 3095, 3096 for receiving a fastener that connects the inner and outer frame portions 3072, 3074 together. The distal ends 3094 are connected together at inner end 3083. The arms 3082 can be curved such that the distal ends 3094 extend above at least a portion of the remainder of the arms 3082. For example, in the illustrated example, the arms 3082 include curved portions 3084. The inner end portion 3083 of the distal ends of the arms 3082 are connected to the distal ends 3091 of the arms 3080 of the inner frame portion 3072 such that the distal ends 3091, 3094 can move together in the proximal direction PD or the distal direction DD.

In some implementations, the arms 3082 are more flexible than the arms 3080. This increased flexibility allows the arms 3082 to flex when the connection portion 3083 is pulled into the arms 3080. This flexing allows the arms 3082 to narrow. The stiffer arms 3080 allow the paddles of the device to open and closed in the same or a similar manner to that shown in FIGS. 23, 27, and 30-37.

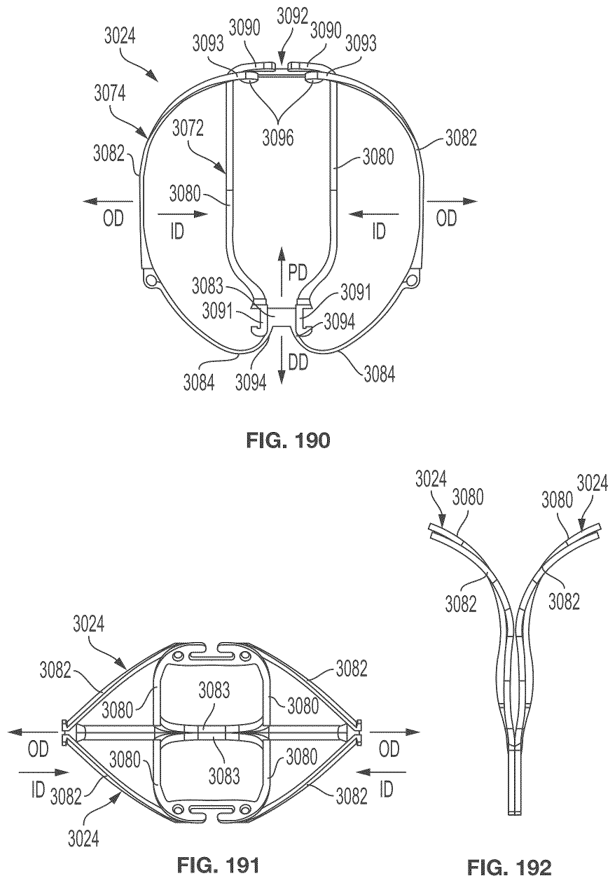

Referring to FIG. 190, movement of the inner end portion 3083 that connects the distal ends 3091, 3094 of the arms 3080, 3082 in the distal direction DD causes the arms 3082 to move in the outward direction OD (FIG. 190) such that the paddle frame 3024 is in an expanded position. That is, referring to FIG. 190, movement of the inner end portion 3083 in the distal direction DD causes the curved portions 3084 of the arms 3082 to flex outward, which causes the arms 3082 to move in the outward direction OD.

Referring to FIG. 190, movement of the inner end portion 3083 that connects the distal ends 3091, 3094 of the arms 3080, 3082 in the proximal direction PD causes the arms 3082 to move in the inward direction ID such that the paddle frame 3024 moves to a narrowed position. That is, referring to FIG. 190, movement of the inner end portion 3083 in the proximal direction PD causes the curved portions 3084 of the arms 3082 to flex inward, which causes the arms 3082 to move in the inward direction ID.

The inner end portion 3083 can be moved in the distal direction DD or the proximal direction PD by a user with an actuation element (e.g., actuation shaft, wire, etc. 212 shown in FIGS. 22-37). For example, the inner end portion 3083 can be connected to the coupled to the actuation element, such that the actuation element can move the connection wire in the proximal direction PD and the distal direction DD. A wide variety of mechanisms can be used to move the inner end portion 3083 in the proximal and distal directions to adjust the width of the paddle frames. Several examples of mechanisms that can be used to move the inner end portion 3083 in the proximal and distal directions to adjust the width of the paddle frames are disclosed below.

In some implementations, the paddle frames 3024 illustrated by FIGS. 189-192 can have a normal, expanded width between 5 mm and 15 mm, such as between 7 mm and 12 mm, such as between 9 mm and 11 mm, such as about 10 mm. The narrowed width of the paddle frame 3024 can be between 3 mm and 12 mm, such as between 5 mm and 10 mm, such as between 7 mm and 9 mm, such as about 8 mm. A ratio of the normal, expanded width to the narrowed width can be between 10/9 and 3/1, such as between 5/4 and 2/1, such as between 4/3 and 3/2.

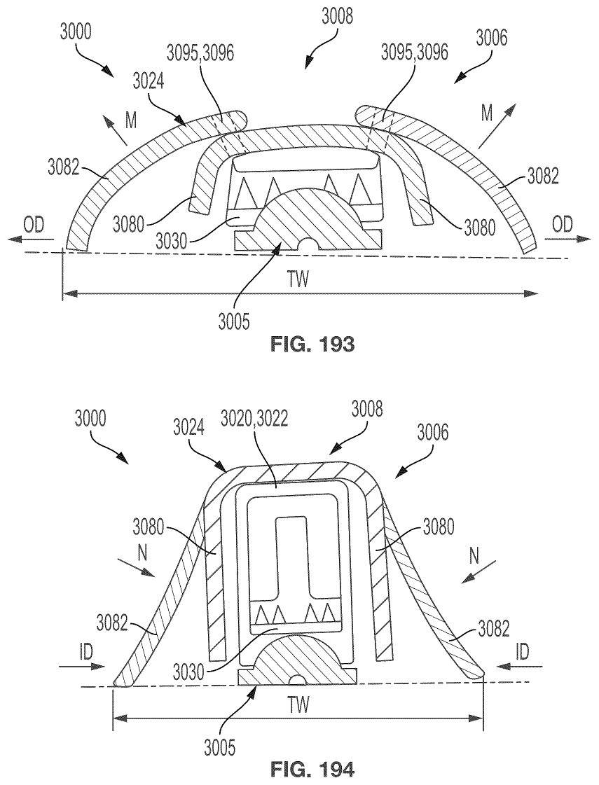

FIGS. 193-195 illustrate an example implementation of a device or implant 3000 where the paddle frames 3024 have a wide configuration (FIG. 193) when the device or implant is closed and the paddle frames 3024 have a narrow configuration (FIG. 194) when the device or implant is closed. In the example illustrated by FIGS. 193-195, the device 3000 includes the paddle frames 3024 of FIGS. 189-192. However, the device 3000 can use a wide variety of different paddle frames that automatically (e.g., due only to the opening and closing of the paddles of the device) move from a narrow configuration when the device is open to a wide configuration when the device is closed. In some implementations, the paddle frames 3024 of the device 3000 are similar to the example illustrated by FIGS. 190-192, except the ends of the arms 3082 are fixed relative to the ends of the arms 3080. For example, ends of the arms 3082 and ends of arms 3080 can both be fixed to a distal cap of the device.

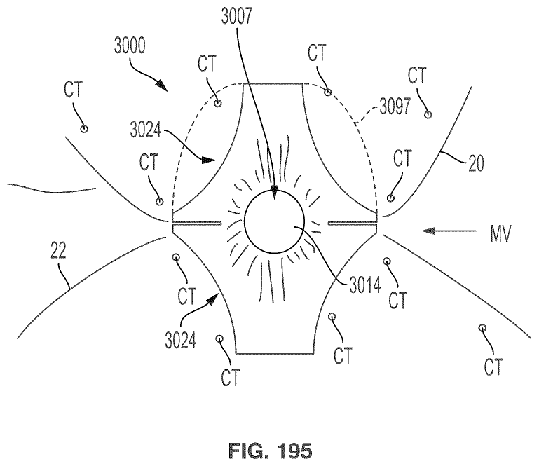

The device or implant 3000 can include a coaptation portion (not shown), a proximal or attachment portion 3005 (FIGS. 193-194), an anchor portion 3006, and a distal portion 3007 (FIG. 195). The coaptation portion, attachment portion 3005, and distal portion 3007 can take any suitable form, such as, for example, the form for these portions of the device 200 shown in FIGS. 22-37, or any other form described in the present application. The device 3000 can include any other features of devices or implants discussed elsewhere in the present application or in the applications and patents incorporated by reference herein, and the device 3000 can be positioned to engage valve tissue 20, 22 as part of any suitable valve repair system (e.g., any valve repair system disclosed in the present application). In addition, any of the devices described herein can incorporate the features of the device 3000.

The anchor portion 3006 of the device 3000 can take any suitable form, such as, for example, the form of the anchor portion 206 of the device 200 shown in FIGS. 22-37 (except that the paddle frame 224 is replaced with the paddle frame 3024 shown in FIGS. 189-195), or any other form described in the present application that can incorporate paddle frame 3024. The anchor portion 3006 can include a plurality of anchors 3008, each anchor 1508 including outer paddles (e.g., outer paddles 220 shown in FIGS. 22-37), inner paddles (e.g., inner paddles 222 shown in FIGS. 22-37), paddle extension members or paddle frames 3024, and clasps 3030 (e.g., clasps 230 shown in FIGS. 22-37).

Referring to FIG. 193, when the device is in the closed position, a stretching or pulling force on the outer paddle arms 3082 is at a minimum. As a result, an outward biasing force M of the more flexible arms is sufficient to flex the arms in the outward direction OD and form a concave shape.

Referring to FIG. 194, when the device is moved toward the open condition, a stretching or pulling force on the outer paddle arms 3082 increases. As a result, width of the arms 3082 decreases in the inward direction ID. The connections between the arms 3082 and the stiffer arms 3080 creates a force N on the arms 3082 that causes the arms 3082 to flex inward and form the convex shape shown in FIG. 194.

Referring to FIG. 195, the device or implant 3000 is shown attached to a native valve (in the illustrated example, to the leaflets 20, 22 of a mitral valve MV). The device 2900 is shown with the paddle frames 3024 in the narrowed position such that the paddle frames 3024 convex shapes which allow the device 3000 to maneuver more easily into position for implantation on the native valve. That is, various chordae tendinea CT are shown surrounding the device 3000, and the convex shape of the paddle frames 3024 allow the device to move within the ventricle with minimized contact with the chordae tendinea CT.

Still referring to FIG. 195, a dashed line 3097 shows the shape of the paddle frame 3024 when in the expanded position with a concave shape. As shown, when the paddle frame 3024 has the concave shape, the device 3000 can have more contact with the chordae tendineae CT. Once the device 3000 is positioned for implantation on the native valve, the paddle frames 3024 can be moved to the expanded position to better secure the anchors 3008 of the device 3000 to the leaflets 20, 22. In addition, as the paddle frames 3024 take the concave shape (shown by dashed line 3097) when being moved to the expanded position, the outer surface of the anchors 3008 may contact some of the chordae tendinea CT.

Referring to FIGS. 193 and 194, the total width TW of the paddle frame 3024 when in the closed/wider position (FIG. 193) can be between 5 mm and 15 mm, such as between 7 mm and 12 mm, such as between 9 mm and 11 mm, such as about 10 mm. The narrowed total width of the paddle frame 3024 when in the open/narrower position can be between 3 mm and 12 mm, such as between 5 mm and 10 mm, such as between 7 mm and 9 mm, such as about 8 mm. A ratio of the normal, expanded width to the narrowed width can be between 10/9 and 3/1, such as between 5/4 and 2/1, such as between 4/3 and 3/2.

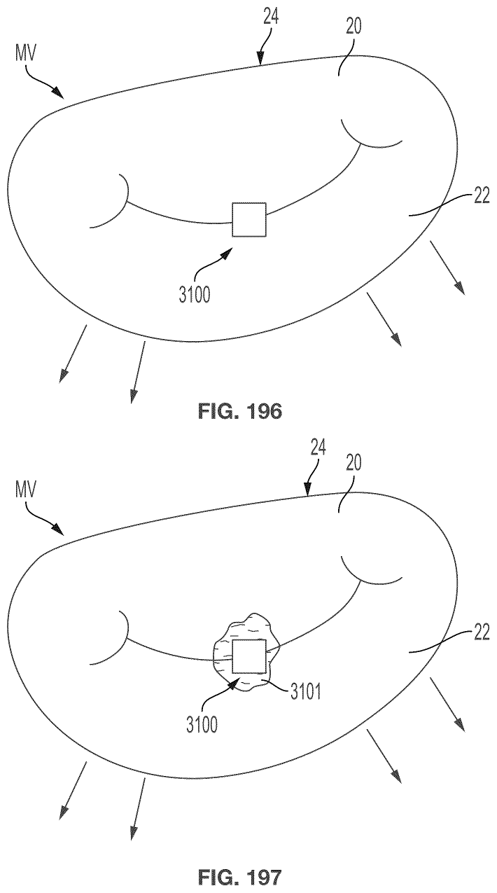

Referring to FIGS. 196-198, an example implementation of a device or implant 3100 on a native valve is shown. The device or implant 3100 can take any suitable form, such as, for example, any form described in the present application or in the applications and patents incorporated by reference herein. In some implementations, the implant or device 3100 includes a coaptation element and in some implementations, the device or implant does not include a coaptation element.

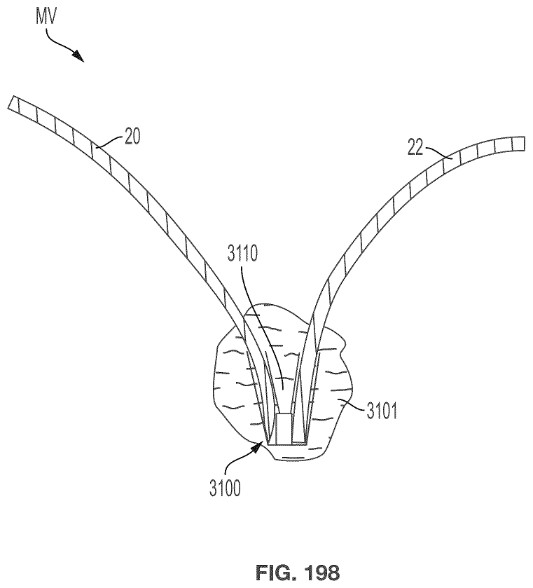

The device 3100 is shown attached, as an example, to the leaflets 20, 22 of the mitral valve MV. After implantation of the device 3100, over time, the annulus 24 of the mitral valve may expand in the outward direction. In particular, the expansion may occur proximate the posterior leaflet 22 of the mitral valve MV. This expansion of the annulus 24 may allow regurgitation of blood from the left ventricle through the mitral valve MV even when the device 3100 is attached to the mitral valve MV. That is, the expansion of the annulus 24 may cause openings proximate the device 3100 that allows blood to regurgitate through the mitral valve MV. Also, over time, tissue ingrowth 3101 (FIGS. 197-198) from the leaflets can cover the device 3100, which provides additional support to secure the device 3100 to the mitral valve leaflets.

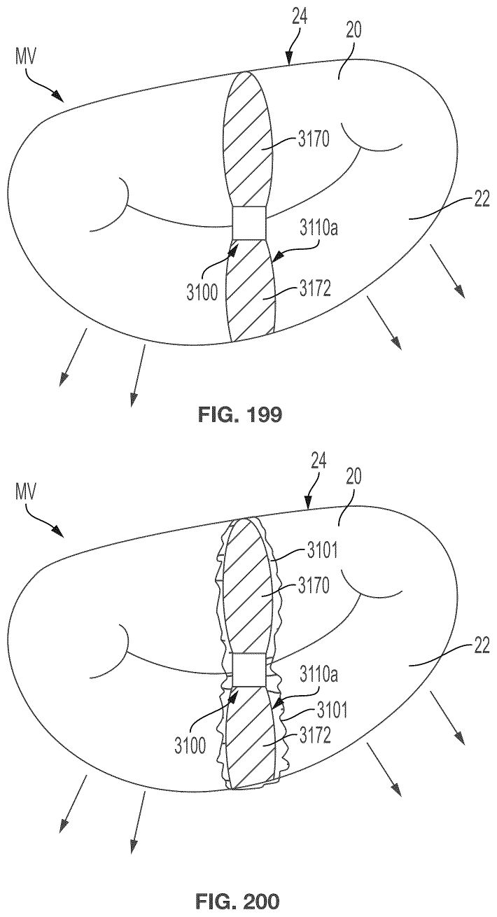

Referring to FIGS. 199-209, in some implementations, the device 3100 can include a tissue bridge member 3110a (e.g., fabric, mesh, tissue, etc.). The tissue bridge member 3110a can be a separate component that is attached to any other portion of the device 3100, a component that is integral to the device 3100, or a component that is attached to any portion of the device 3100 after implantation of the device 3100 on the native valve. The tissue bridge member 3110a can be configured to prevent or inhibit expansion of the annulus 24 when tissue ingrowth 3101 covers the device 3100 and the tissue bridge member 3110a. The tissue bridge member 3110a prevents or inhibits expansion of the annulus 24 because the tissue bridge member 3110a and/or tissue ingrowth 3101 extends to the annulus 24 of the native valve. That is, the tissue ingrowth 3101 spans or bridges from one side of the annulus 24 to the other side of the annulus to prevent or inhibit the bridged sides of the annulus 24 from pulling apart.

Referring to FIGS. 199-200, in some implementations, the tissue bridge member 3110a can include a first extension portion 3170 that extends to across the anterior leaflet 20 of the mitral valve MV to the annulus 24 and a second extension portion 3172 that extends across the posterior leaflet 22 to the annulus 24. Each extension portion 3170, 3172 can be equally sized or can be sized to fit or extend along each of the native valve leaflets. For example, the leaflets 20, 22 are typically different sizes and the extension portions 3170, 3172 can have different sizes that correspond to the leaflets 20, 22. The tissue ingrowth 3101 connects the extension portions 3170, 3172 to the annulus 24 to prevent or inhibit the expansion of the annulus 24. The extension portions 3170, 3172 can be separate components or can be a single component.

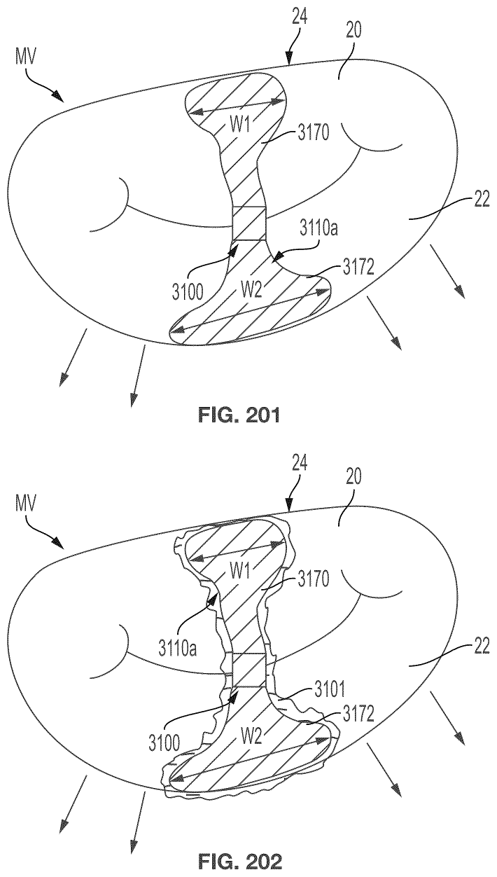

Referring to FIGS. 201-202, in some implementations, the tissue bridge member 3110a can include a first extension portion 3170 that extends to across the anterior leaflet 20 of the mitral valve MV to the annulus 24 and a second extension portion 3172 that extends across the posterior leaflet 22 to the annulus 24. The first extension portion 3170 can have a first width W1, and the second extension portion can 3172 have a second width W2 that is greater than the first width W1. The widths W1, W2 can be selected to minimize or control expansion of the annulus. For example, if it is determined that the annulus 24 is likely to expand or expand more proximate the posterior leaflet 22, the second extension portion 3172 have a greater width than the first extension portion 3170. This wider width provides additional support to the annulus 24 in the area that is more likely to expand when the tissue ingrowth 3101 covers the device 3100. As with the example of FIGS. 199 and 200, the extension portions 3170, 3172 can be equally sized or can be sized to fit or extend along each of the native valve leaflets. For example, the extension portions 3170, 3172 can have different sizes that correspond to the leaflets 20, 22. The tissue ingrowth 3101 connects the extension portions 3170, 3172 to the annulus 24 to prevent or inhibit the expansion of the annulus 24. The extension portions 3170, 3172 can be separate components or can be a single component.

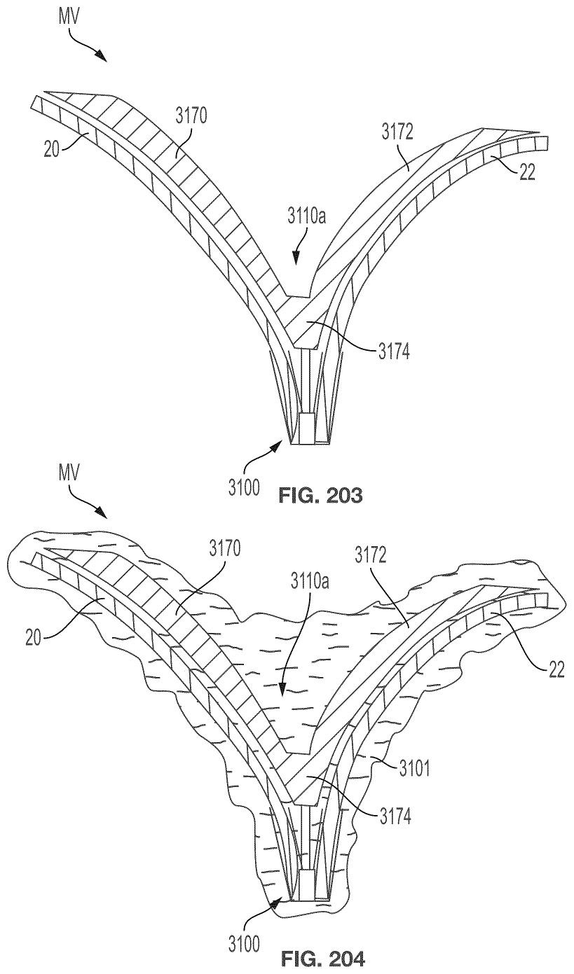

Referring to FIGS. 203-204, in some implementations, the tissue bridge member 3110a has a V-shape such that the first extension portion 3170 and the second extension portion 3172 are connected at a connection portion 3174. The connection portion 3174 is attached to the device or implant 3100. In this example, the first and second extension portions 3170, 3172 can be equally sized or the first and second extension portions 3170, 3172 can be sized differently. The tissue bridge 3101 forms over the first and second extension portions 3170, 3172 and fills in a portion of the “V.”

Referring to FIGS. 205-206, in some implementations, the tissue bridge member 3110a has a triangular shape. The first extension portion 3170 and the second extension portion 3172 are connected at a connection point 3174 at the vertex of the triangular shape. The connection portion 3174 is attached to the device 3100. In this example, the first and second extension portions 3170, 3172 can be equally sized or the first and second extension portions 3170, 3172 can be sized differently. The tissue bridge 3101 forms over the bridge member 3110a.

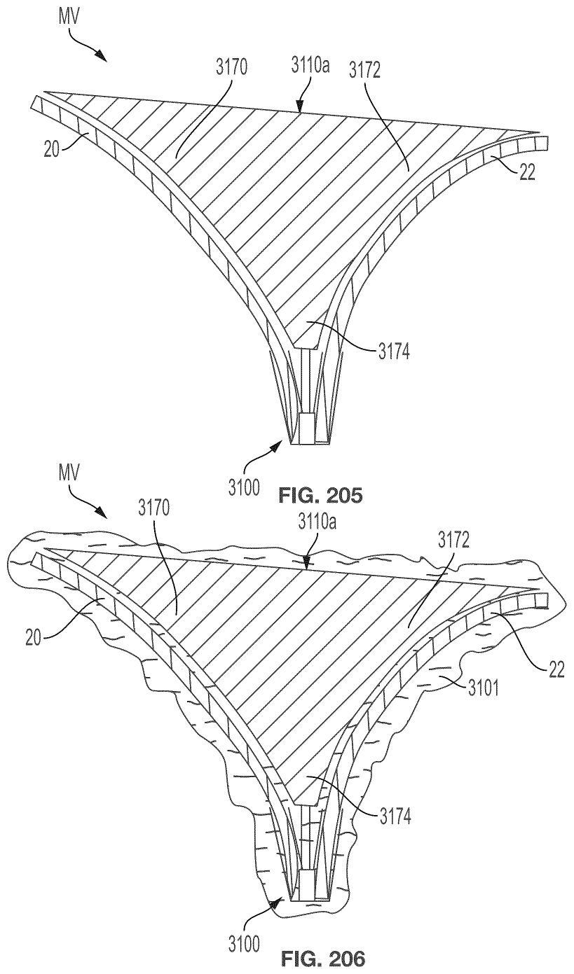

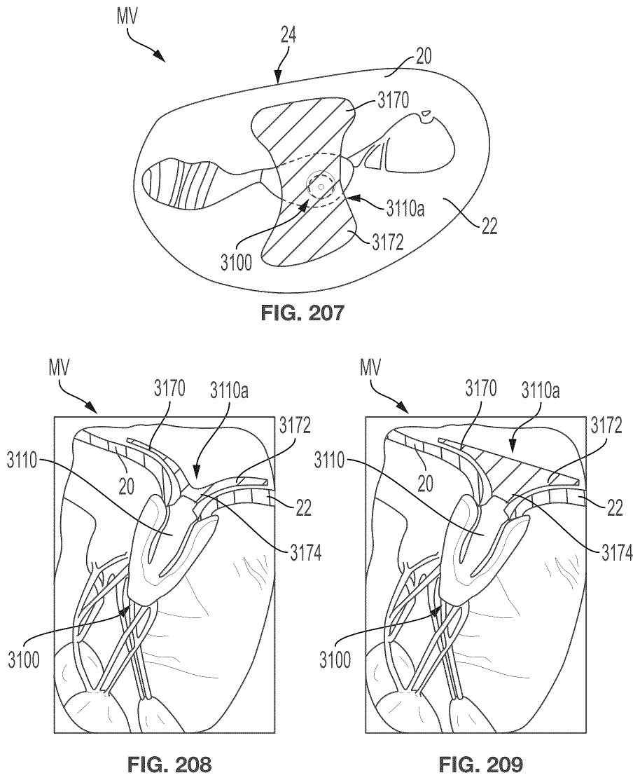

Referring to FIGS. 207-209, in some implementations, the tissue bridge member 3110a extends from an optional coaptation element or spacer 3110 of the device or implant 3100. In the illustrated example, the bridge member does not extend entirely to the annulus 24. However, the tissue bridge member 3110a can take any of the forms of FIGS. 199-204 and can extend all the way to the annulus 24 on one or both sides. As shown in FIG. 207, the extension portions 3170, 3172 of the tissue bridge member 3110a can be equally sized. Alternatively, the extension portion 3172 extending over the posterior leaflet 22 can have a greater width than the extension portion extending over the anterior leaflet 20. In addition, referring to FIG. 208, tissue bridge member 3110a can have a V-shape (e.g., similar to the example shown in FIG. 203-204), or, referring to FIG. 209, tissue bridge member 3110a can have a triangular shape (e.g., similar to the example shown in FIGS. 205-206).

Referring to FIGS. 199-209, tissue bridge member 3110a can be made of any suitable material that promotes tissue ingrowth. For example, tissue bridge member 3110a can be made of a biocompatible material. The tissue bridge member 3110a can be made of a loosely knit or woven cloth material.

While the disclosed examples show that the extension portions 3170, 3172 can be equally sized or the extension portion 3172 can have a greater width than the extension member 3170, in some implementations, the extension portion 3170 can have a greater width than the extension member 3172. Also, while the disclosed examples show the coaptation extension member 3110a having a V-shape or a triangular shape, it should be understood that the coaptation extension member 3110a can have any suitable shape that prevents or inhibits the annulus 24 from expanding when tissue ingrowth 3101 covers the device 3100.

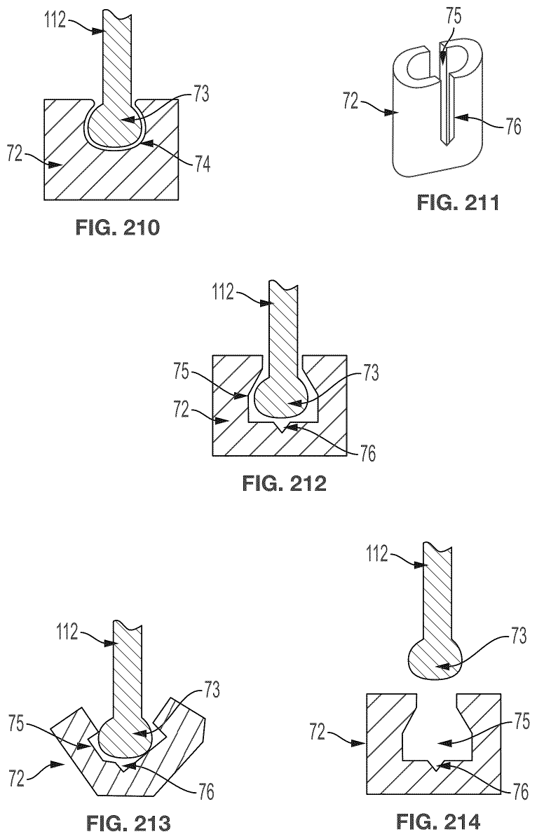

FIGS. 210-275 show various configurations for engaging and disengaging the retention end 73 of the actuation element 112 to a retention feature 72 on the cap 214 or collar 211 of the device 200 (See, e.g., FIG. 23) or another component of the device. However, the cap, collar, and/or device 200 can have any of the configurations disclosed in the present patent application. A wide variety of different configurations can be used. The retention end and/or the retention feature can be tapered, have one or more features that can flex inward and spring back outward, have cutting or impaling surfaces or features, and/or have guide surfaces. The retention features can be provided on the cap 114, 214 (See, e.g., FIG. 23), the collar 211, and/or another recapturing component. The retention features can be made from a variety of different materials. As shown in FIGS. 210-214, the retention end 73 of the actuation element 112 can engage with a retention feature 72 on the cap 214 or collar 211 of the device 200 using a ball and socket connection. In some implementations, the retention end 73 of the actuation element 112 is spherical and made of somewhat elastic material. The retention end 73 of the actuation element 112 fits within a socket 74 in the retention feature 72 of the cap 214 or collar 211. The socket 74 is spherical with an opening slightly smaller than the diameter of the retention end 73 of the actuation element 112, such that the socket 74 must deform slightly in order to receive the retention end 73 of the actuation element 112. In some implementations, the retention feature 72 is made of somewhat elastic material.

FIGS. 211-214 show an example implementation of the retention feature 72 having an orifice 75 for receiving an actuation element 112, and at least one longitudinal slit 76 extending downward from the orifice 75. In some implementations, there are two slits 76 on either side of the orifice. FIG. 212 shows the retention end 73 of the actuation element 112 within the orifice 75 of the retention feature 72. In some implementations, the diameter of the top of the orifice 75 is smaller than the diameter of the spherical portion of the retention end 73 of the actuation element 112, such that the orifice 75 must bend open slightly to receive the retention end 73 of the actuation element 112. FIG. 213 shows the retention feature 72 in an open configuration, wherein the slits 76 on either side of the orifice 75 allow for the expansion of the retention feature 72 when the actuation element 112 is acted upon with an upward force greater than the operating force. FIG. 214 shows the actuation element 112 completely disengaged from the retention feature 72.

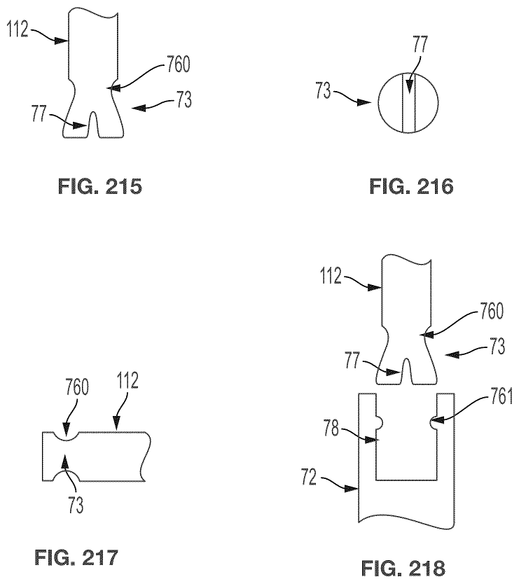

The retention end 73 of the actuation element 112 can have one or more features which allow it to flex inward to engage with the retention feature 72. FIGS. 215-222 show an example of the retention end 73 of the actuation element 112 and the retention feature 72, wherein the retention end 73 has at least one relief cut 77 allowing for the compression and expansion of the retention end 73 within the retention feature 72. FIG. 215 shows the retention end 73 of the actuation element 112 having a single relief cut 77 through the bottom surface. In some implementations, the retention end 73 can be any shape, such as spherical, bulbous, or tapered. The retention end 73 can have a tapered portion 760 having a lesser diameter than the remainder of the retention end 73. FIG. 216 shows a bottom view of the retention end 73 of the actuation element 112 having a single relief cut 77. FIG. 217 shows a side view of the retention end 73 of the actuation element 112, wherein the tapered portion 760 of the retention end 73 has a lesser diameter than the remainder of the retention end 73. FIG. 218 shows the actuation element 112 and the retention feature 72 in a disengaged configuration. The retention feature 72 has an orifice 78 for receiving the retention end 73 of the actuation element 112. In some implementations, the inside of the orifice 78 has a ridge 761. The retention end 73 of the actuation element 112 can be advanced into the retention feature 72 and the ridge 761 can force the retention end 73 of the actuation element 112 to compress inward at the relief cut 77. This allows the retention end 73 of the actuation element 112 to enter the lower portion of the orifice 78. At which point, the retention end 73 can expand outward such that the ridge 761 of the retention feature 72 rests within the tapered portion 760 of the retention end 73. The actuation element 112 is held within the retention feature 72 by the outward force of the retention end 73 against the walls of the orifice 78 until the actuation element 112 is pulled upward with a force greater than the operating force, causing the retention end 73 to compress against the ridge 761 and disengage from the retention feature 72.

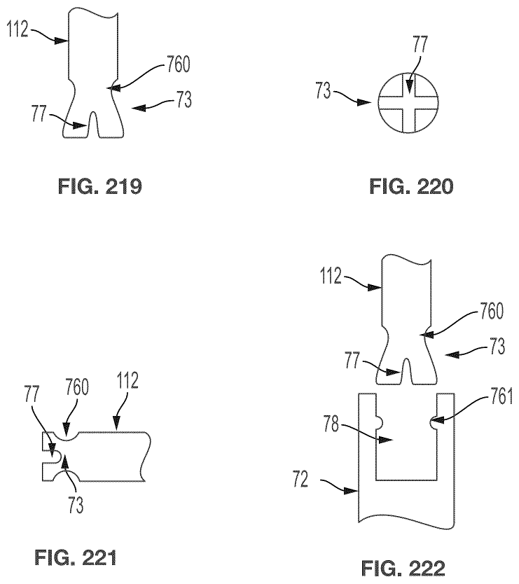

FIGS. 219-222 depict an example of the actuation element 112 and retention feature 72, wherein the retention end 73 of the actuation element 112 has two perpendicular relief cuts 77 through the bottom surface. The retention end 73 of the actuation element 112 can have any number of relief cuts 77 and configurations thereof.

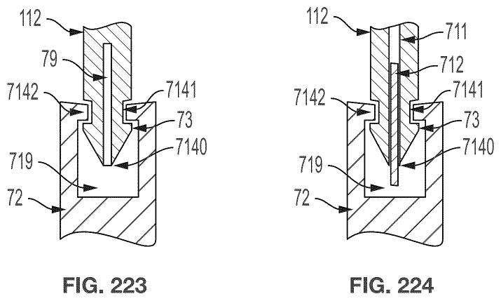

FIGS. 223 and 224 show an example of the actuation element 112 engaging with the retention feature 72, wherein the retention end 73 of the actuation element 112 has a tapered tip 7140. FIG. 223 shows the actuation element 112 having a tapered tip 7140, a longitudinal relief passage and/or cut 79, and a shelf 7141. The retention feature 72 has an orifice 719 and a lip 7142 at the top of the orifice 719. In this implementation, the tapered tip 7140 can be advanced through the orifice 719 of the retention feature. The lip 7142 will cause the retention end 73 to compress inwards towards the longitudinal relief passage and/or cut 79, allowing the tapered tip 7140 of the retention end 73 to enter the orifice 719.

When the lip 7142 becomes proximate to the shelf 7141 at the end of the tapered tip 7140, the retention end 73 will expand within the orifice 719. The expanded retention end 73 will hold the actuation element 112 in place within the retention feature 72 until the actuation element is pulled upward with a force greater than the operating force as to compress the retention end 73 against the lip 7142, allowing the actuation element to disengage from the retention feature 72.

FIG. 224 shows the actuation element 112 having a tapered tip 7140, a shelf 7141, and a passage 711 through which a rod 712 can be inserted. In this implementation, the tapered tip 7140 can be advanced through the orifice 719 of the retention feature. The lip 7142 will cause the retention end 73 to compress inwards towards the longitudinal passage 711, allowing the tapered tip 7140 of the retention end 73 to enter the orifice 719. When the lip 7142 becomes proximate to the shelf 7141 at the end of the tapered tip 7140, the retention end 73 will expand within the orifice 719.

A rod 712 can be advanced through the passage 711 to the retention end 73 of the actuation element 112. The rod 712 can further expands the tapered tip 7140 within the orifice 719 or hold the tapered tip in the expanded position. The expanded retention end 73 and the rod 712 hold the actuation element 112 in place within the retention feature 72 until the rod 712 is retracted from the retention end 73 of the actuation element 112, and the actuation element 112 is pulled upward with a force, greater than the operating force, to compress the retention end 73 against the lip 7142, and allow the actuation element 112 to disengage from the retention feature 72.

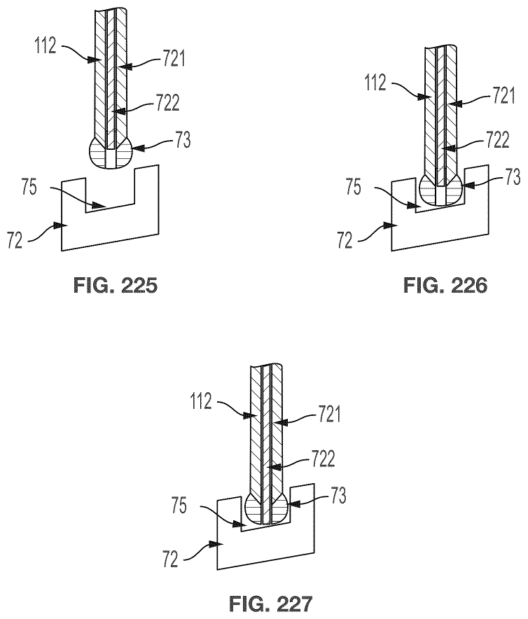

The actuation element 112 can have one or more features, such as a rods or wires, which can be advanced through a passage into the tip of the actuation element 112 while the actuation element 112 is engaged with the retention feature 72 of the cap 214 or collar 211, in order to secure the tip within the retention feature 72. FIGS. 225-227 show an example implementation of the actuation element 112 and a retention feature 72, wherein the actuation element 112 has a passage 711, a rod 712 extending through said passage 711, and a retention end 73. In some implementations, the retention end 73 is spherical and made of elastomeric material. The retention end 73 can be made of a variety of different materials and shapes, such as tapered, pointed, or spherical. As shown in FIG. 225, the actuation element 112 can be advanced into an orifice 75 of the retention feature 72, with the rod 712 not yet advanced into the retention end 73. The orifice 75 can be a variety of shapes, such as rectangular, cylindrical, or tapered. As shown in FIG. 226, the retention end 73 of the actuation element 112 engages with the retention feature 72. In some implementations, the diameter of the retention end 73 can be substantially similar, or slightly less than, the diameter of the orifice 75. The retention end 73 can be made of an elastomeric material such that it compresses slightly to engage with the orifice 75 of the retention feature 72. As shown in FIG. 227, the rod 712 can be advanced through the passage 711 into the retention end 73 to provide structure to the retention end 73, such that the actuation element 112 is held in place within the retention feature 72 until the rod 712 is retracted from the retention end 73 of the actuation element 112, and the actuation element 112 is pulled upward with such a force, greater than the operating force, as to compress the retention end 73.

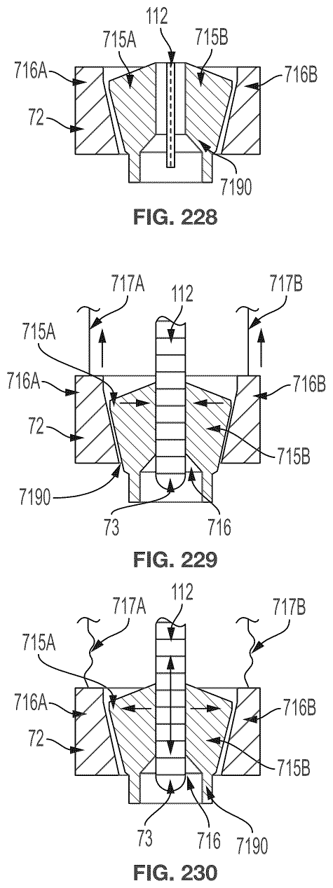

As shown in FIGS. 228-230, the actuation element 112 can also engage with the retention feature 72 using a collet connection. FIG. 228 shows an example implementation of the retention feature 72 comprising a first and second tapered outer portion 716A, 716B. A locking mechanism 7190, comprising first and second inner tapered portions 715A, 715B and a central passage 716, rests within a recess formed by the first and second tapered outer portions 716A, 716B. As shown in FIG. 229, an actuation element 112 having a retention end 73 can be advanced through the central passage 716 of the locking mechanism 7190. The first and second tapered outer portion 716A, 716B can be pulled upwards by increasing tension on first and second tethers 717A, 717B. When the first and second tapered outer portions 716A, 716B move upwards, their tapered surfaces engage the first and second inner tapered portions 715A, 715B of the locking mechanism 7190, which compresses the actuation element 112 therein and thereby connect the actuation element to the locking mechanism. As shown in FIG. 230, when the tension on the first and second tether 717A, 717B decreases, the first and second tapered outer portions 716A, 716B move downward and away from the surface of the first and second inner tapered portions 715A, 715B, which allows the locking mechanism to splay open and release the actuation element 112.

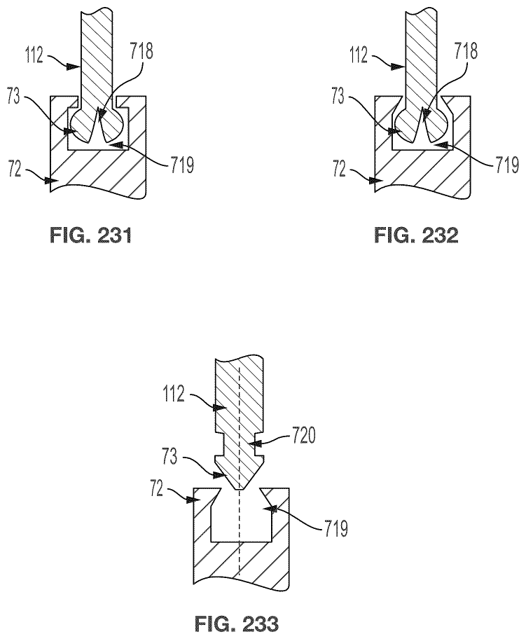

The orifice or orifices in the retention feature 72 can be tapered, have one or more features such as lips or bevels, and/or have guide surfaces which can hold the retention end 73 of the actuation element 112 in place within the retention feature 72 until a sufficient upward force greater than the operating force is applied to the actuation element 112. As shown in FIGS. 231-233, any combination of examples of the retention end 73 and the retention feature 72 can be used. As shown in FIG. 231, the orifice 719 can be cylindrical having a lip with of a lesser diameter at the opening of the orifice 719. The retention end 73 of the actuation element 112 can have a longitudinal cut 718 through the center of the retention end 73, such that the retention end 73 splays open slightly. The actuation element 112 can be advanced into the orifice 719 of the retention feature 72, wherein the lips of the orifice 719 force the retention end 73 to compress, closing the gap caused by the cut 718, and allowing the retention end 73 to fit through the lip of the orifice 719 having a lesser diameter than the remainder of the orifice 719. Once the retention end 73 is engaged within the orifice 719 of the retention feature 72, the retention end expands from the cut 718 such that the diameter of the retention end 73 is larger than the diameter of the lip of the orifice 719. The actuation element 112 will be held in place within the retention feature 72 until a sufficient upward force greater than the operating force is placed on the actuation element 112 to cause the lip of the orifice 719 to compress the retention end 73 of the actuation element 112. As shown in FIG. 232, the lip of the orifice 719 can be tapered or beveled, such that the diameter of the orifice 719 gradually decreases towards its opening.

FIG. 233 shows an example implementation of the retention feature 72 and the retention end 73 of the actuation element 112. The lip of the orifice 719 is tapered, or beveled, such that the diameter of the orifice 719 gradually decreases towards its opening. The retention end 73 of the actuation element 112 is a tapered tip, having a shelf 720 of a lesser diameter than the remainder of the retention end 73. The retention end 73 can be engaged with the retention feature 72, such that the lip of the orifice 719 rests within the shelf 720 of the actuation element 112, holding the retention end 73 in place within the orifice 719 until a sufficient upward force greater than the operating force is placed on the actuation element 112.

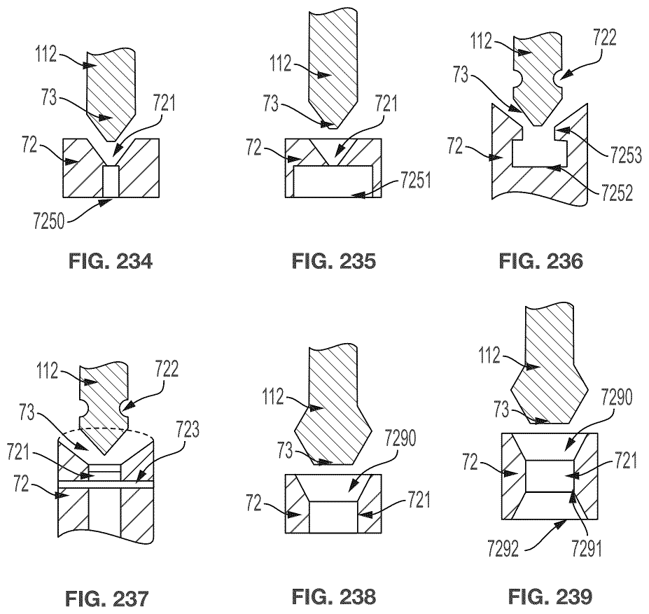

The retention feature 72 can engage with the retention end 73 of the actuation element 112 via a friction fit connection. The retention end 73 can be conical, tapered, beveled, or otherwise shaped to fit within a similarly shaped orifice of the retention feature 72. The retention feature 72 and the retention end 73 can be made of such materials as to enhance the friction fit, such as elastomeric materials or materials having a threaded or knurled surface. FIGS. 234-244 show some implementations of the retention end 73 of the actuation element 112 and the retention feature 72, wherein the retention end 73 engages the retention feature 72 via a friction fit connection.

FIG. 234 shows an actuation element 112 having a retention end 73 that is beveled with a tip having a lesser diameter than the actuation element 112. The retention feature 72 has an orifice 721 which is beveled such that the retention end 73 fits tightly within the orifice 721. In some implementations, the retention feature 72 can have a channel 7250 which allows for the slight expansion and compression of the retention feature 72 when engaged with the retention end 73, such as to further hold the retention end 73 in place within the retention feature 72. FIG. 235 shows a wide channel 7251 below the beveled orifice 721.

FIG. 236 shows an example implementation of the retention end 73 and the retention feature 72, wherein the retention feature 72 comprises an orifice 7252 having a beveled entrance and a lip 7253 of lesser diameter than the remainder of the orifice 7252. The retention end 73 of the actuation element 112 has a tapered tip, having a shelf 722 of lesser diameter than the remainder of the retention end 73 and actuation element 112. The retention end 73 can engage with the retention feature 72, such that the retention end 73 is held via friction fit inside the orifice 7252. The lip 7253 allows the retention features 72 to expand slightly around the tip of the retention end 73.

FIG. 237 shows an example implementation of the retention end 73 and the retention feature 72, wherein the retention feature 72 comprises an orifice 721 with a beveled entrance and a band 723, such as an elastic band, surrounding the exterior of the retention feature 72. The retention end 73 of the actuation element 112 has a tapered tip, having a shelf 722 of lesser diameter than the remainder of the retention end 73 and actuation element 112. The retention end 73 of the end of the actuation element 112 can be advanced into the orifice 721 of the retention feature 72, such that the band 723 expands to allow the engagement of the retention end 73 within the orifice 721, and then contracts to hold the retention end 73 in place.

The retention end 73 of the actuation element 112 can be of a variety of shapes, such as beveled, tapered, spherical, and the like. As shown in FIGS. 238 and 239, the retention end 73 can be rectangular with beveled ends, such that the top and bottom portions of the retention end 73 are of a lesser diameter than the remainder of the retention end 73. FIG. 238 shows a retention feature 72 having an orifice 721 with a beveled entrance 7290, such that the retention end 73 can fit tightly within the beveled entrance 7290 of the orifice 721. The orifice 721 allows the retention feature 72 to expand slightly, and then compress around the retention end 73 when it engages with the entrance 7290.

FIG. 239 shows a retention feature 72 having an orifice 721 with a beveled entrance 7290, a middle portion 7291, and a beveled bottom portion 7292. The retention end 73 of the actuation element 112 can be advanced into the orifice 721 such that when the beveled portion of the retention end 73 contacts to the beveled entrance 7290 of the orifice 721, the retention end 73 causes the middle portion 7291 of the orifice 721 to expand to accept the retention end 73. The retention end 73 of the actuation element 112 advances through the expanded middle portion of the orifice 721 into the beveled bottom portion 7292. The beveled top of the retention end 73 fits tightly within the beveled bottom portion 7292 of the orifice 721, holding the actuation element 112 within the retention feature 72.

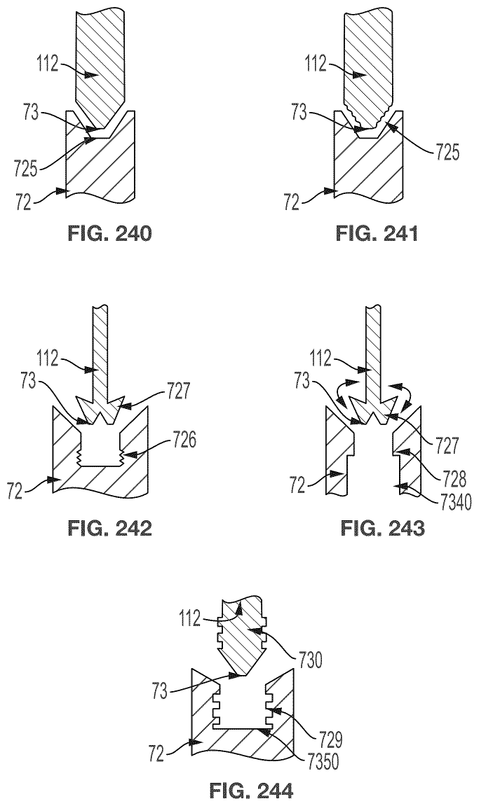

In some implementations, the retention end 73 and/or the retention feature 72 can optionally be made of an elastomer material with a knurled or threaded surface to enhance the friction fit between the retention end 73 and the retention feature 72. The retention end 73 and retention feature 72 can form various male/female connections, such as beveled, tapered, spherical, or pointed ends and similarly shaped orifices. FIG. 240 shows the retention end 73 of an actuation element 112 wherein the retention end 73 is tapered. The retention feature 72 has an orifice 725 tapered to fit the retention end 73 tightly. The retention end 73 and retention feature 72 can optionally be made of an elastomer material to enhance the friction fit therebetween. FIG. 241 shows the retention end 73 having a knurled or threaded surface to further enhance the friction fit between the retention end 73 and the orifice 725 of the retention features 72. The orifice 725 can also have a knurled or threaded surface.

FIG. 242 shows an example implementation of the retention end 73 of the actuation element 112 and the retention feature 72, wherein the retention end 73 comprises two flexible wings having a notch therebetween. The retention feature 72 has a tapered entrance and an orifice 726 having a threaded surface. The retention end 73 can be inserted into the orifice 726, wherein the beveled entrance forces the wings 727 of the retention end 73 to flex upward such that the retention end 73 can fit inside the orifice 726. Once the retention end 73 is within the orifice 726, the wings 727 expand against the threaded surface of the orifice 726. The friction between the threaded surface of the orifice 726 and the expansion of the wings 727 against the orifice 726 holds the retention end 73 in place within the retention feature 72. When a sufficient upward force greater than the operating force is applied to the actuation element 112, the wings 727 of the retention end 73 flex downward, releasing the friction fit of the retention end 73 against the orifice 726, and allowing the retention end 73 to be removed from the retention feature 72.

FIG. 243 shows the retention end 73 of FIG. 242 and a retention feature 72 having a beveled entrance, an orifice 728 having a lower portion 7340 with a greater diameter than the remainder of the orifice 728. The actuation element 112 can be advanced into the orifice 728 of the retention feature 72, wherein the beveled entrance causes the wings 727 of the retention end 73 to flex upward allowing the retention end 73 to enter the orifice 728. Once the retention end 73 reaches the lower portion 7340 of the orifice 728, the wings 727 can expand out within the lower portion 7340 of the orifice 728 holding the actuation element 112 in place within the retention feature 72. When a sufficient upward force greater than the operating force is applied to the actuation element 112, the wings 727 of the retention end 73 flex downward, releasing the friction fit of the retention end 73 against the lower portion 7340 of the orifice 728, and allowing the retention end 73 to be removed from the retention feature 72.

In some implementations, the surface of the retention feature 72 and/or the surface of the retention end 73 have a plurality of threads or knurls to enhance the friction fit therebetween. FIG. 244 shows an example implementation of a retention end 73 and a retention feature 72, wherein the surface of the retention end 73 has a plurality of threads 730 and the surface of the orifice 729 within the retention feature 72 has a plurality of threads 7350. The retention end 73 can be tapered or pointed. The retention feature 72 can have a beveled entrance to guide the retention end 73 into the orifice 729. The retention end 73 can be advanced into the orifice 729, such that the threaded surface 730 of the retention end 73 comes in contact with the threaded surface 7350 of the orifice 729. The contact between the threaded surfaces 7350, 730 create a friction fit between the retention end 73 and the retention feature 72, holding the retention end 73 within the orifice 729 until a sufficient upward force greater than the operating force is placed on the actuation element 112.

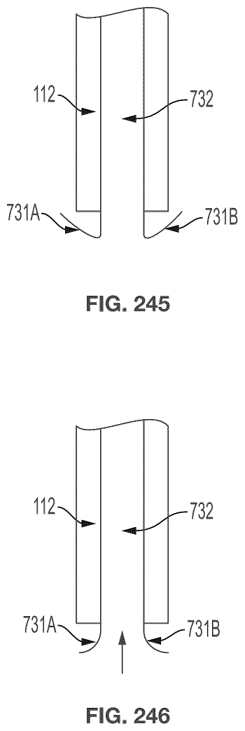

The actuation element 112 can comprise one or more features, such as rods, hooks, or lumens, made of shape-memory alloys, such as Nitinol. These features can be advanced into a retention feature 72 in order to hold the actuation element 112 in place. The features can be retracted or otherwise removed from the retention feature 72 to allow for the removal of the actuation element 112 therefrom. FIGS. 245-250 show an example implementation of an actuation element 112 and a retention feature 72, wherein the actuation element 112 has an inner passage 732 and at least one retention extension 731A, 731B. The retention extension(s) 731A, 731B can take a wide variety of different forms. For example, the retention extensions 731A, 731B can be formed by cutting and shaping an end of a tube, by shaping a wire, etc. The actuation element 112 can have first and second retention extensions 731A, 731B, as shown in FIG. 245. However, any number of retention extensions can be included. The first and second retention extensions 731A, 731B can be made of a shape-memory alloy, such as Nitinol.

When the ends of the first and second retention extensions 731A, 731B are extended beyond the end of the actuation element 112, the ends are biased to bend upward in a hook-like configuration. As shown in FIG. 246, when the first and second retention extensions 731A, 731B are completely retracted into the passage 732 they return to a substantially straight configuration.

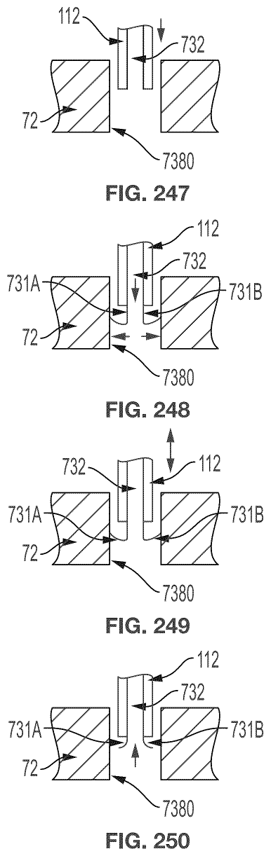

FIGS. 247-250 depict the process of securing the actuation element 112 of FIGS. 245 and 246 to a retention feature 72. In this example, the actuation element 112 has a passage 732, and first and second retention extensions 731A, 731B. The retention feature 72 has an orifice 7380 through which the actuation element 112 can be advanced. FIG. 247 shows the actuation element 112 entering the orifice 7380 of the retention feature 72. FIG. 248 shows the first and second retention extensions 731A, 731B advancing through the passage 732 in the actuation element 112. As the first and second retention extension 731A, 731B extend out of the actuation element 112, they change from a substantially straight configuration to a hooked configuration. The hooked portions of the retention extensions 731A, 731B extend outward to the surface of the orifice 7380.

As shown in FIG. 249, the force of the hooked retention extensions 731A, 731B against the orifice 7380 holds the actuation element 112 in place within the retention feature 72. As shown in FIG. 250, the actuation element 112 can be disengaged from the retention feature 72 by retracting the retention extensions 731A, 731B into the passage 732 of the actuation element 112, where the retention extensions 731A, 731B are returned to their substantially straight configuration.

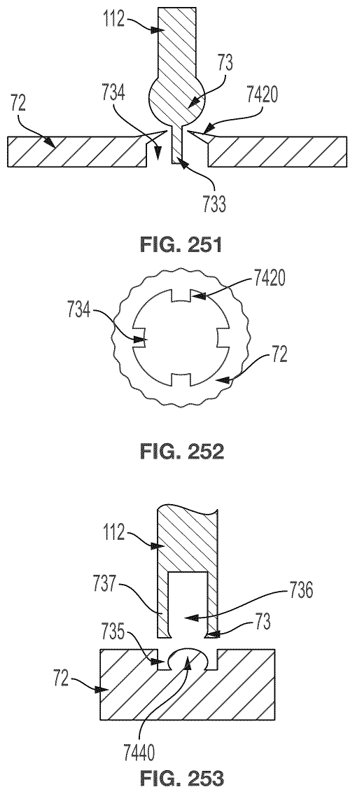

The actuation element 112 can engage with the retention feature through various male/female connections such as snaps, prongs, and the like. FIGS. 251-253 some implementations of snap fit connections between a retention end 73 of the actuation element 112 and the retention feature 72. As shown in FIG. 251, the actuation element 112 has a retention end 73 comprising a cylindrical tip 733 of a substantially smaller diameter than the actuation element 112. The retention feature 72 can comprise an orifice 734 having a plurality of prongs 7420 at its opening. The prongs extend towards the center of the orifice 734, such that when the actuation element 112 is advanced into the orifice 734 the ends of the prongs 7420 contact the surface of the retention end 73. The friction fit between the prongs 7420 and the retention end 73 hold the actuation element 112 in place within the retention feature 72, until the actuation element 112 is acted on with sufficient upward force to overcome the friction forces therebetween. FIG. 252 shows a top view of the retention feature 72. In some implementations, the orifice 734 has four prongs 7420. However, any number of prongs can be used.

FIG. 253 shows an example implementation of the actuation element 112 and the retention feature 72. The retention feature can comprise a recessed portion 735 with a raised button 7440 therein. The actuation element 112 can have a retention end 73 having a recessed portion 736 and at least one prong 737. The actuation element 112 can engage the retention feature 72 by advancing the prongs 737 into the recessed portion 735 of the retention feature 72, such that the prongs 737 snap into place around the raised button 7440. The snap fit between the prongs 737 and the raised button 7440 hold the actuation element 112 to the retention feature 72. The actuation element 112 can be disengaged from the retention feature 72 by pulling upward on the actuation element 112 with sufficient force greater than the operating force to release the prongs 737 from the raised button 7440. In some implementations, the actuation element 112 can comprise the recessed portion and the button, whereas the retention feature 72 can comprise the prongs.

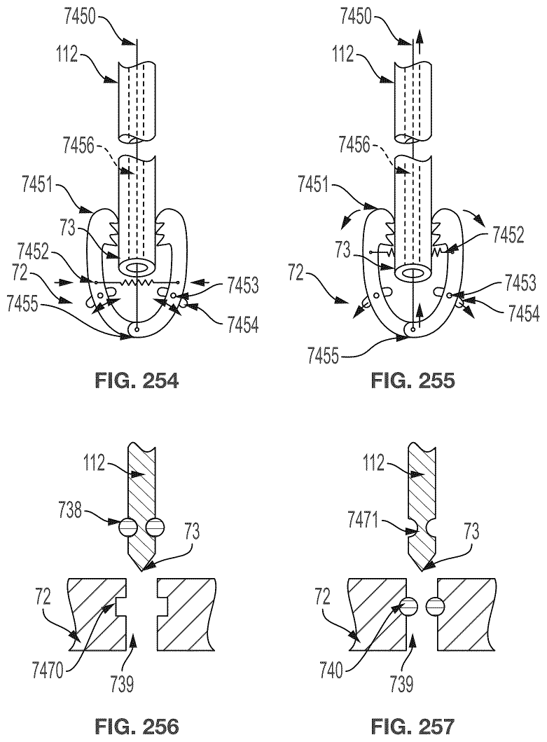

The retention feature 72 and actuation element 112 can engage each other using a clasp mechanism. FIGS. 254-255 show an example implementation of the actuation element 112 and a retention feature 72 comprising a clasp 7451 operated by a clasp line 7450. As shown in FIG. 254, the retention feature 72 has a clasp 7451 biased to a closed position via a spring 7452. The clasp 7451 is attached to the retention feature 72 via at least one pin 7453 within at least one slot 7454, such that when the clasp 7451 moves between an open and closed position, the pins 7453 slide along the slots 7454 allowing the clasp 7451 to open and close without decoupling from the retention feature 72. The actuation element 112 comprises a retention end 73 and a passage 7456 through which a clasp line 7450 extends. The clasp line 7450 extends through the actuation element 112 and is releasably connected, such as by looping, knotting, or flossing, to the clasp hinge 7455. The clasp 7451 is biased via the spring 7452 to remain closed against the sides of the actuation element 112. As shown in FIG. 255, when tension is applied to the clasp line 7450, the clasp line 7450 pulls upward on the clasp hinge 7455. The upward movement of the clasp hinge 7455 causes the pins 7453 to slide along the slots 7454 and allows the clasp 7451 to open away from the actuation element 112. The clasp line 7450 can be released from the clasp hinge 7455 such as by removing the loop, untying the knot, unflossing the line from around the clasp hinge, or any similar means. The actuation element 112 can then be disengaged from the retention feature 72.

The retention feature 72 and/or the actuation element 112 can comprise features such as O-rings, gaskets, elastic surfaces, or various other means to increase the friction fit therebetween. FIGS. 256-261 show some implementations of the retention end 73 of the actuation element 112 and the retention feature 72, where at least one of the retention end 73 and the retention feature 72 has a recessed O-ring 738, 740. FIG. 256 shows an actuation element, a retention end 73, and a recessed O-ring 738 proximate to the retention end 73. The retention feature 72 has an orifice 739 having a radial notch 7470 of a greater diameter than the remainder of the orifice 739. The actuation element 112 can advance into the orifice 739, and the recessed O-ring 739 is compressed between the surface of the actuation element 112 and the surface of the orifice 739. When the recessed O-ring 738 reaches the notch 7470 it expands therein, securing the retention end 73 of the actuation element 112 inside the orifice 739.

FIG. 257 shows an example implementation of the actuation element 112 and the retention feature 72, wherein the retention feature 72 has a recessed O-ring 740 inside its orifice 739, and the actuation element 112 has a retention end 73 having a notch 7471 with a slightly lesser diameter. The retention end 73 of the actuation element 112 can be inserted into the orifice 739 of the retention feature 72 such that once the notch 7471 is proximate to the recessed O-ring 740 within the orifice 739, the actuation element 112 is held with the retention feature 72 due to the friction fit between the recessed O-ring 740 and the actuation element 112.

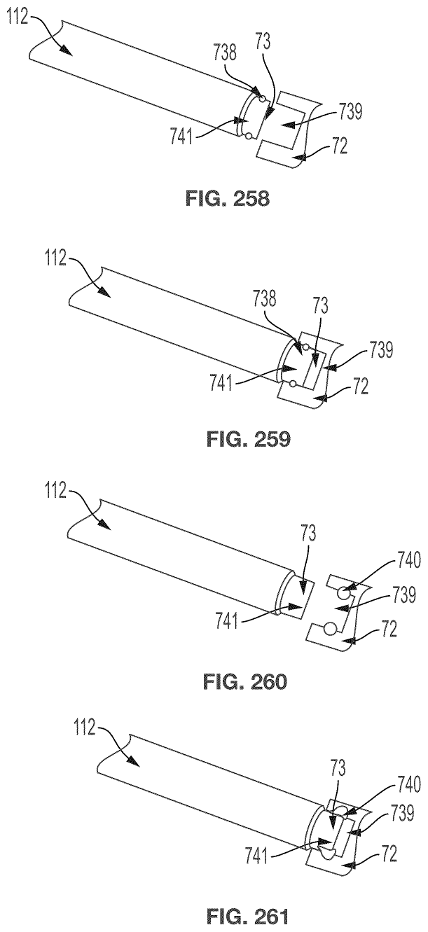

FIGS. 258 and 259 show an example implementation of an actuation element 112 and a retention feature 72, wherein the actuation element 112 has a retention end 73 and a recessed O-ring 738 around a peg portion 741 of the retention end 73 having a lesser diameter than the remainder of the actuation element 112. In some implementations, the retention feature 72 has an orifice 739 with a knurled or threaded surface. As shown in FIG. 259, the peg portion 741 of the retention end 73 can be advanced into the orifice 739 of the retention feature 72. The o-ring 738 around the peg portion 741 will compress to allow the peg portion 741 to enter the orifice 739. A combination of the friction force between the compressed recessed O-ring 738 and the knurled or threaded surface of the orifice 739 will hold the retention end 73 of the actuation element 112 within the retention feature 72 until an upward force greater than the operating force is applied to the actuation element 112, causing the retention end 73 to disengage from the retention feature 72.

Alternatively, the recessed O-ring 740 can be inside the orifice 739 of the retention feature 72, and the retention end 73 can have a knurled or threaded surface. FIGS. 260 and 261 show an example implementation wherein the actuation element 112 has a retention end 73 having a peg portion 741 of a lesser diameter than the remainder of the actuation element 112. The peg portion 741 has a knurled or threaded outer surface. The retention feature 72 has an orifice 739 having a recessed O-ring 740 therein. As shown in FIG. 261, the actuation element 112 can engage with the retention feature 72, by advancing the peg portion 741 of the retention end 73 into the orifice 739 such that the peg portion 741 compresses the recessed O-ring 740. The friction forces between the knurled or threaded outer surface of the peg portion 741 and the compressed, recessed O-ring 740 hold the retention end 73 of the actuation element 112 in place within the retention feature 72 until an upward force greater than the operating force is applied to the actuation element 112, causing the retention end 73 to disengage from the retention feature 72.

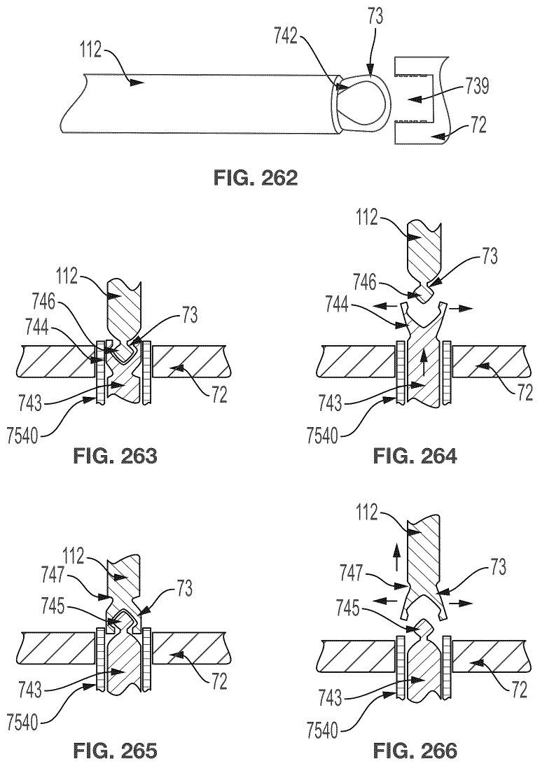

The retention end 73 can have various features, such as tapered tips, beveled edges, bulbs, and rings, which can advance into an orifice within the retention feature 72. FIG. 262 shows a retention end 73 having a ringed portion 742. In some implementations, the ringed portion 742 can be made of a firm, yet flexible elastomer material. The retention feature 72 has an orifice 739 with a knurled or threaded surface. The ringed portion 742 of the retention end 73 can be advanced into the orifice 739. The ringed portion 742 must deform slightly to fit therein, such that the friction forces between the orifice 739 and expanding ringed portion 742 hold the retention end 73 of the actuation element 112 in place within the retention feature 72.

The actuation element and the retention feature can engage one another via a coupling connection between a flexible clasp and a bulb. The flexible clasp can be made of a shape-setting material, such as Nitinol, which can be biased in an open or closed position. The bulb can be part of the retention end 73 of the actuation element 112 or the retention feature 72. The flexible clasp can be part of the retention end 73 or the retention feature 72. FIGS. 263 and 264 show an example implementation of a retention end 73 of an actuation element 112 and a retention feature 72. The retention feature 72 comprises a sleeve 7540 and a coupler 743 housed therein. The coupler 743 has a flexible clasp 744. The coupler 743 is allowed limited longitudinal movement but is biased towards a recessed position within the sleeve 7540, such as by a spring or other flexible material. As shown in FIG. 263, an actuation element 112 having a retention end 73 with a bulb tip 746 can be inserted into the flexible clasp 744 of the coupler 743. As shown in FIG. 264, when the actuation element 112 is acted on in an upward force greater than the operating force, the actuation element 112 pulls the coupler 743 out of the sleeve 7540. The coupler 743 can be made of a shape-setting material, such as Nitinol, which can bias the flexible clasps 744 in an open position wider than the diameter of the sleeve 7540. When the end of the coupler 743 is beyond the opening of the sleeve 7540, the flexible clasps 744 expand outward releasing the bulb tip 746 of the retention end 73.

Alternately, as shown in FIGS. 265 and 266, the bulb portion of the engagement mechanism can be part of the retention feature 72, and the flexible clasps can be part of the actuation element 112. FIG. 265 shows a retention feature 72 comprising a sleeve 7540 and a coupler 743 housed therein. The coupler 743 has a bulb tip 745. The coupler 743 is allowed limited longitudinal movement but is biased towards a recessed position within the sleeve SS540, such as by a spring or other flexible material. An actuation element 112 having a retention end 73 with flexible clasps 747 can be inserted onto the bulb tip 745 of the coupler 743. The retention end 73 can be made of a shape-setting material, such as Nitinol, which can bias the flexible clasps 747 in an open position wider than the diameter of the sleeve 7540. As shown in FIG. 266, when an upward force greater than the operating force is placed on the actuation element 112, the flexible clasps 747 expand outward releasing the bulb tip 746 of the coupler 743.

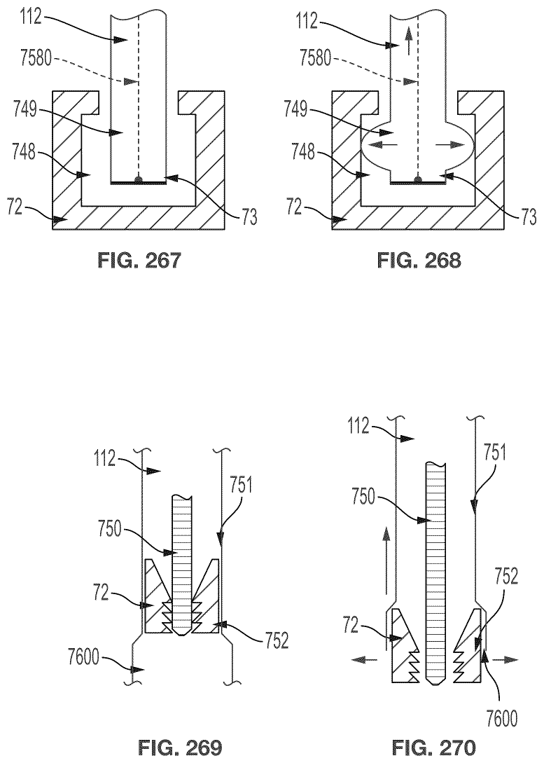

The retention end 73 of the actuation element 112 can include a portion that can be expanded by the user, such as by inflation, compression, collapsing of material, or by control element (e.g., wire, etc.) operation. FIGS. 267 and 268 show an example implementation of the actuation element 112 having a control wire 7580 and a retention end 73 with an expanding portion 749. The retention feature 72 has an orifice 748 to engage with the retention end 73. The orifice 748 can be beveled, lipped, or tapered, such that the diameter of the opening of the orifice 748 is smaller than that of the remainder of the orifice 748. As shown in FIG. 267, the retention end 73 of the actuation element 112 can be advanced into the orifice 748. As shown in FIG. 268, control wire 7580 is secured to the tip of the retention end 73 of the actuation element 112. Increasing tension on the control wire 7580 causes the expanding portion 749 to collapse longitudinally and extend outward towards the sides of the orifice 748. This effect can be achieved by creating an area of increased flexibility, such as by using relief cuts or thin-walled material. The increased diameter of the collapsed, expanding portion 749 of the retention end 73 within the orifice 748 prevents or inhibits removal of the retention end 73 from the retention feature 72. When tension is removed from the control wire 7580, the expanding portion 749 can return to a substantially straight configuration, such that the actuation element 112 can disengage from the retention feature 72.

FIGS. 269 and 270 show an example implementation of a retention end 73 and a retention feature 72, wherein retention feature 72 has at least one toothed clamp 752. The actuation element 112 comprises a central rod 750 within a sheath 751 having a beveled end portion 7600 of a larger diameter than the remainder of the sheath 751. As shown in FIG. 269, the actuation element 112 can engage with the retention feature 72 such that the central rod 750 is captured by toothed clamp(s) 752 within the sheath 751. The diameter of the sheath 751 is such that it compresses the toothed clamps 752 against the central rod 750, securing the actuation element 112 to the retention feature 72. As shown in FIG. 270, the actuation element 112 can be disengaged from the retention feature 72 by pulling upwards on the sheath 751 independent from the central rod 750. Once the sheath 751 is positioned such that the beveled end portion 7600 is proximate clasp, the toothed clamp(s) 752 can expand outward away from the central rod 750. The actuation element 112 is then free to disengage from the retention feature 72.

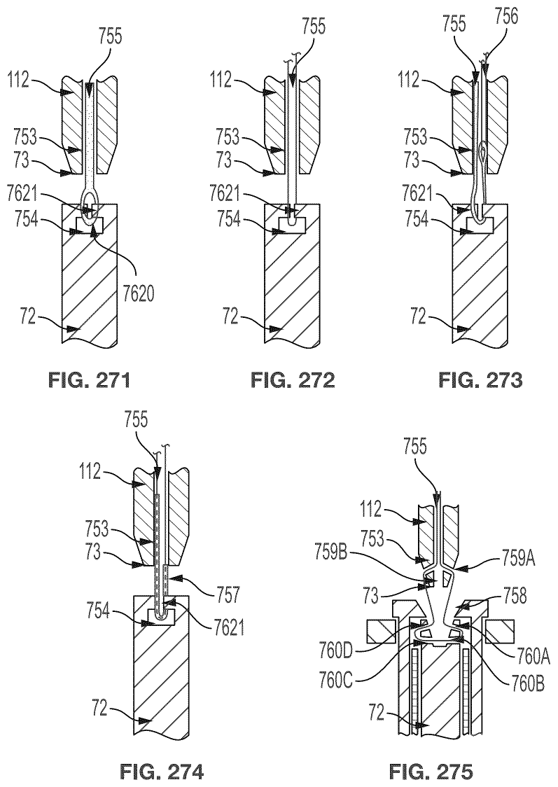

In some implementations, the actuation element 112 can engage with the retention feature 72 using loops, hooks, pulls, tethers, control wires, or other means of manual engagement. In some implementations, the use of loops, hooks, or the like, provide a primary and secondary method of disengagement. As shown in FIG. 271-275, the actuation element 112 can comprise a control element (e.g., wire, etc.) which can be threaded, looped, or otherwise inserted into a retention feature 72. FIG. 271 shows an actuation element 112 having a retention end 73 and a passage 753 containing a control wire 755. The retention feature 72 has an orifice 754 having a closure 7621 at its entrance. The closure 7621 almost entirely covers the orifice 754 except for a gap slightly smaller than the diameter of the control wire 755. The end of the control wire 755 has a loop 7620 such that the loop 7620 can deform slightly to advance through the closure 7621 and into the orifice 754. When sufficient upward force is placed upon the control wire 755, the loop 7620 can be removed through the closure 7621, and the actuation element 112 can disengage from the retention feature 72.

As shown in FIGS. 272-274, the control wire 755 can be threaded through the orifice 754. FIG. 272 shows an actuation element 112 having a retention end 73 and a passage 753 containing a looped control wire 755. The retention feature 72 has an orifice 754 having a closure 7621 at its entrance. The closure 7621 almost entirely covers the orifice 754 except for a gap slightly smaller than the diameter of the control wire 755. The control wire 755 extends down through the passage 753 and then upwards through the passage 753 forming a loop. The looped control wire 755 can be threaded through the orifice 754 or the loop can deform slightly to advance through the closure 7621 of the orifice 754. When sufficient upward force is placed upon the control wire 755, the loop can be removed through the closure 7621, and the actuation element 112 can disengage from the retention feature 72. Additionally, the control wire 755 can be removed by increasing tension to one side of the control wire 755 in order to pull the control wire 755 through the orifice 754 and through the passage 753.

Similarly, as shown in FIG. 273, the actuation element 112 can comprise a hooked portion 756 within the passage 753. One end of the control wire 755 can be secured to the hooked portion 756, such as by looping or knotting. When sufficient upward force is placed upon the control wire 755, the loop can be removed through the closure 7621, and the actuation element 112 can disengage from the retention feature 72. Additionally, the control wire 755 can be removed by decoupling the end of the control wire 755 from the hooked portion 756 in order to pull the control wire 755 through the orifice 754 and through the passage 753.

As shown in FIG. 274, the control wire 755 can be fortified by a flexible lumen 757 proximate to orifice 754 and closure 7621 of the retention feature 72. The flexible lumen 757 can provide strength to the control wire 755 to allow it to deform and snap into place through the closure 7621 and into the orifice 754.

FIG. 275 shows an example implementation of a retention end 73 and a retention feature 72, wherein the control wire 755 is threaded through a plurality of orifices within the retention feature 72. The actuation element 112 comprises a retention end 73, and a passage 753 through which a control wire 755 extends. The retention end 73 has a first and second orifice 759A, 759B. The retention feature 72 has a beveled recess 758 having a first, second, third, and fourth orifice 760A, 760B, 760C, 760D. The actuation element 112 can engage with the retention feature 72 such that the retention end 73 fits tightly within the beveled recess 758. The control wire 755 extends through the passage 753, out the first orifice 759A, through the first orifice 760A of the retention feature, through the second orifice 760B of the retention feature, through the third orifice 760C of the retention feature, through the fourth orifice 760D of the retention feature, through the second orifice 759B of the retention end 73, and through the passage 753. A combination of the control wire 755 and the friction fit between the retention end 73 and the beveled recess 758 secure the actuation element 112 to the retention feature 72.

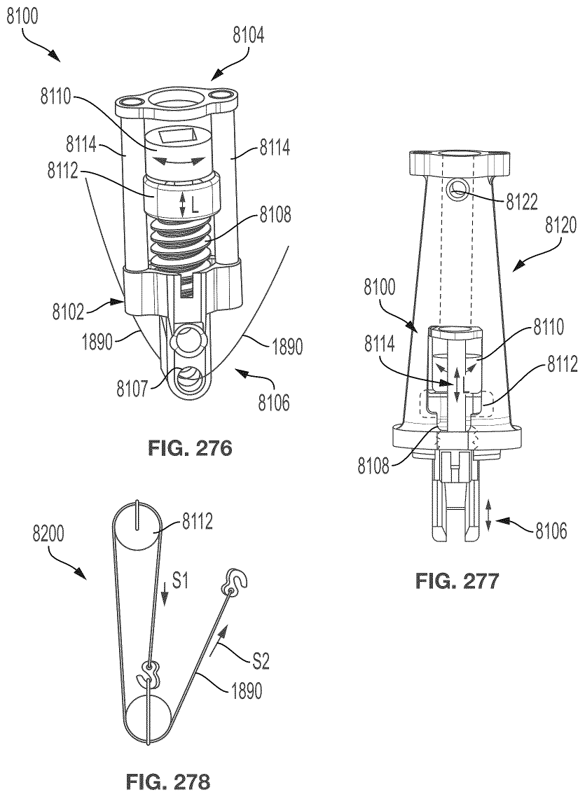

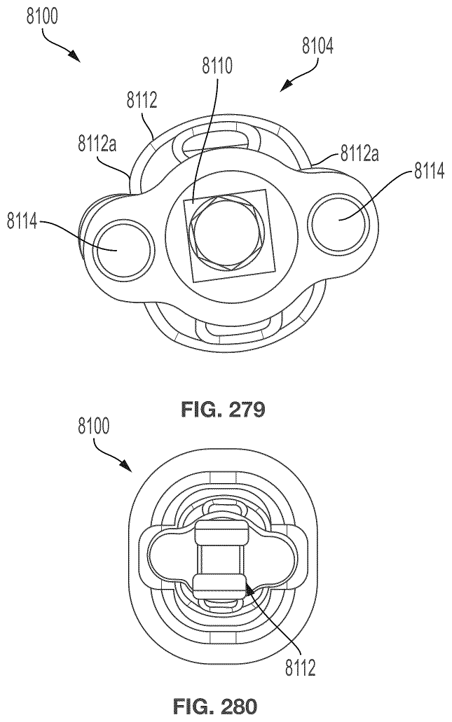

Referring now to FIGS. 276, 279, and 280, an example implementation of a width adjustment device 8100 is shown. FIG. 276 is a perspective view of the width adjustment device 8100, FIG. 279 is a top view of the device, and FIG. 280 is a bottom view of the device. The width adjustment device 8100 is configured to expand and contract in length to expand and contract the paddle frames of a device or implant. For example, any of the device or implants described herein can incorporate features of the width adjustment device 8100. In some implementations, the width adjustment device 8100 can be mechanically coupled to the paddle frames, the distal cap, or to any other suitable attachment point described herein. In this way, it is appreciated that a wide variety of arrangements can be used to adjust the width of the paddle frames. In some implementations, the width adjustment device 8100 is configured to cause the paddle frames to contract inwards to narrow the width of the paddle frames (i.e., contracted position), or expand outwards to increase the width of the paddle frames (i.e., expanded position). The width adjustment device 8100 is particularly suitable for narrowing the width of the paddle frames and paddles of a device or implant (e.g., any of the devices described herein) when navigating through the native structure of the heart—e.g., chordae tendineae.

Referring to FIG. 276, the width adjustment device 8100 can include a receiver, such as the illustrated support body 8102 having a proximal end 8104 and a distal end 8106. In some implementations, the support body 8102 can be an integral part of a device or implant. For example, the support body 8102 can be integrally formed with the distal cap or any other suitable member described in the present application.

Still referring to FIG. 276, a coupler, such as the illustrated externally threaded shaft 8108, is interposed between the proximal and distal ends 8104 and 8106 and is rotatably coupled with the support body 8102. The externally threaded shaft 8108 can take any suitable form, such as, for example, a screw, a bolt, a fastener, or the like. The shaft 8108 can be formed to include a driver head 8110 that is configured to enable rotation of the shaft 8108 by a variety of tools (e.g., various drive types). In the illustrated example, the driver head 8110 is integrally formed with the shaft 8108 as a single, unitary component. However, it is appreciated that the driver head 8110 can be removably attached to the shaft 8108, such as, for example, when the driver head is an independent fastener (e.g., a threaded nut). In the illustrated example, the driver head 8110 is shown as having a square-shaped drive type. However, it is appreciated that a wide variety of drive types can be used (e.g., Torx, slotted, Philips, etc.). An end of the shaft 8108 that is opposite to the head 8110 can be configured to couple the shaft 8108 to the body 8102 to allow the shaft to rotate relative the body without longitudinally moving the shaft relative to the body (e.g., the shaft only spins relative to the body).

Still referring to FIG. 276, the width adjustment device 8100 can include a follower 8112 that has internal threads that mate with external threads of the shaft 8108. Referring to FIG. 279, the follower 8112 can be formed to include an oblong-shaped body having rotation prevention faces 8112a (see FIG. 279). The torque prevention faces 8112a are configured to slide along the columns 8114 of the support body 8102 such that the follower 8112 cannot rotate or rotation is impeded. As such, the follower is constrained to an upward or downward motion along a longitudinal axis L of the shaft 8108 (i.e., axial direction of the shaft 8108). For example, when the driver head 8110 is rotated clockwise (right-handed thread configuration), the threads of the shaft 8108 will cause the internally threaded follower 8112 to move downwards along the shaft 8108. Similarly, when the driver head 8110 is rotated counterclockwise, the follower 8112 will move upwards along the shaft 8108.

In some implementations, the follower 8112 can be mechanically coupled to the distal cap (e.g., 214), a distal portion of the paddle frames, the paddles, or to any other suitable attachment point described in the present application. In this way, the paddle frames can be connected to the follower 8112 such that the paddle frames will either expand or contract as the follower 8112 moves along the shaft 8108. For example, moving the follower 8112 in a downward direction along the shaft 8108 can cause the paddle frames to contract, thereby decreasing the width of the paddle frames. However, it is appreciated that a wide variety of configurations are contemplated. For example, in an alternative configuration, it is appreciated that the paddle frames can expand when the follower is moved in a downward direction along the shaft 8108.

In some implementations, the width adjustment device 8100 can include a line 1890 that is configured to pull or provide slack on the paddle frames (e.g., via paddle frame attachment points; see, e.g., 1892 in FIG. 130) for causing the paddle frames to contract when tension is applied to the line 1890. The line 1890 can take a wide variety of forms, such as, for example, a line, a suture, a wire, a rod, a catheter, or the like. While the examples described herein refer to a single line 1890 for adjusting both paddle frames simultaneously, it is appreciated that each paddle frame could be independently adjusted, such as, for example, when two width adjustment devices 8100 are operated independently relative to each other.

In some implementations, the line 1890 can be coupled to the follower 8112 by an attachment means, such as, for example, a hook, a loop, or any other suitable attachment means described in the present application. Still referring to FIG. 276, tension can be applied to the line 1890 when the follower is moved along the longitudinal axis L of the shaft 8108. In this respect, the position of the follower 8112 along the longitudinal axis L could correspond to a magnitude of tension that is applied to the line 1890, and a corresponding width of the paddle frames. For example, when the driver head 8110 is rotated clockwise, the follower 8112 will move down the shaft 8108 and increase tension that is applied to the line 1890 (e.g., by the pulling of the line). However, it is appreciated that in some implementations, the width adjustment device 8100 can be configured to apply tension to the line 1890 when the driver head 8110 is turned counterclockwise and the follower 8112 is moved up the shaft 8108.

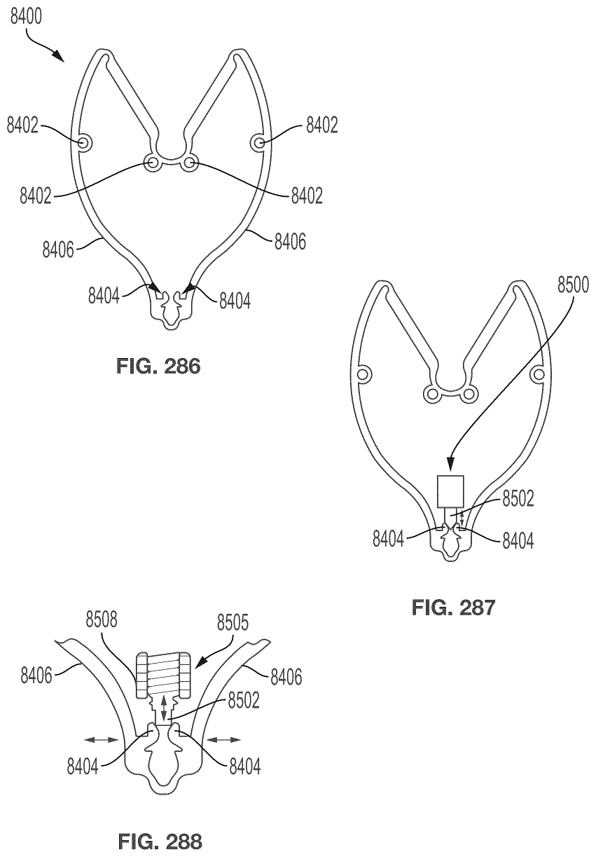

Still referring to FIG. 276, the line can be extended through an aperture 8107 formed in the distal end 8106 of the support body 8102. Opposite ends of the line 1890 can be secured to various attachment points (e.g., 8402 of FIG. 286) on the paddle frames, the paddles, the distal cap, or to any other suitable attachment point described in the present application. While the illustrated example shows the line 1890 extending through the distal end 8106 of the width adjustment device 8100, it should be understood that a wide variety of arrangements are contemplated. For example, the line 1890 can be coupled to a proximal end of the width adjustment device 8100.

The linear expanding and contracting of the device 8100 can be translated to expansion and contraction of the paddle frames in a wide variety of different ways. In FIG. 276, the line 1890 is simply passed through the opening or aperture. In FIG. 277, the line 1890 or other control member can be extended through an opening 8122 of a housing 8120 and the opening can be connected to another part, such as the cap, of the device or implant. The line can be configured to multiply or reduce the movement of the width adjustment device 8100 applied to expand or contract the paddle frames. For example, in FIG. 278 a generated translational motion S1 of the follower 8112 of the device can be doubled to an adjusted motion S2 when a two-pulley system 8200 is utilized. In this manner, the corresponding rate at which the paddle frames open or close could be doubled. While the illustrated example shows a two-pulley arrangement, it is appreciated that any suitable type of arrangement can be used, such as, for example, a three, a four, or a five-pulley arrangement that multiplies or divides the movement of the device.

In some implementations, tension can be applied to the line 1890 by moving the support body 8102 relative to the follower 8112 when the follower 8112 is fixed in place. For example, the follower 8112 can be an integral part of the device or implant (e.g., a part of the distal cap, etc.), and the support body 8102 can be configured to move (e.g., slidably) relative to the follower 8112. In this manner, tension can be applied to the line 1890 when the distal end 8106 of the support body 8102 is moved relative to the follower 8112.

Referring to FIG. 277, an example where the follower 8112 is fixed to the device or implant is shown. In the illustrated example, the width adjustment device 8100 is identical to the example shown in FIG. 276, except that the width adjustment device 8100 is disposed in a separate housing 8120. The housing 8120 can take any suitable form that facilitates attachment of the width adjustment device 8100 to the device or implant.

In the example illustrated by FIG. 277, the follower 8112 is shown as being entrapped/fixed within the housing 8120. In this way, the housing 8120 is harnessed to the follower 8112. Thus, when the driver head 8110 is rotatably adjusted to move the shaft 8108 upwards or downwards, the corresponding movement of the follower 8112 will cause the housing 8120 to also move along the longitudinal axis L of the shaft 8108.

Opposite ends of the line 1890 can be connected to any of the suitable paddle frame attachment points described herein. In such implementations, when the driver head 8110 is rotatably driven, it will cause the follower 8112 and the housing 8120 to move along the longitudinal axis L of the shaft 8108. For example, when rotating the driver head 8110 clockwise (right-handed thread configuration), the follower 8112 and housing 8120 will move downwards along the shaft 8108. As the line 1890 is pulled, the paddle frames will contract inwards toward a closed position. However, it is appreciated that in other configurations, tension can be applied to the line 1890 when the housing 8120 is moved up the longitudinal axis L of the shaft 8108. It is also contemplated that in other configurations, the paddle frames can expand when tension is applied to the line 1890. Therefore, it is appreciated that a wide variety of configurations are contemplated for expanding or contracting the paddle frames.

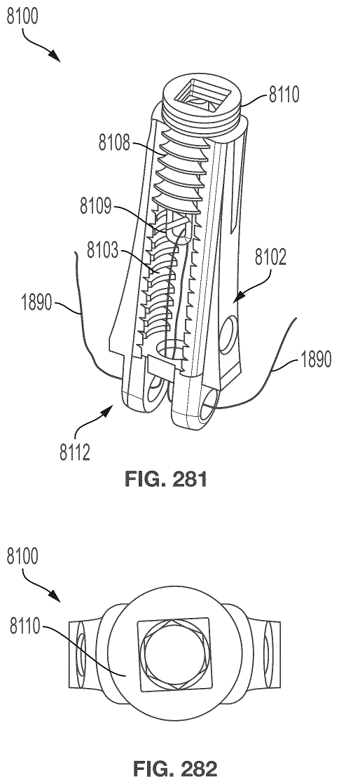

Referring to FIGS. 281 and 282, an example implementation of a width adjustment device 8100. FIG. 281 is a perspective view of the width adjustment device 8100 and FIG. 282 is a top view of the width adjustment device. In this example, the external threads of the shaft 8108 engage internal threads of the body 8102 or a threaded element of the device (e.g., a nut, threaded column, threaded lumen, threaded shaft, threaded pathway, etc.). As such, the shaft both translates and rotates along the body or threaded element. The shaft 8108 can include a connecting portion 8109 for coupling the line 1890 to the shaft 8108. The connecting portion 8109 can take any suitable form, such as, for example, the illustrated ring. In this example, applying torque to the driver head 8110 will cause the threaded shaft 8108 to rotate and longitudinally move in the internally threaded element or column 8103 of the support body 8102. In this manner, the line 1890 is both pulled and twisted by the connecting portion 8109. The tension that is applied to the line 1890 will cause the paddle frames to contract. However, it is appreciated that a wide variety of configurations are possible.

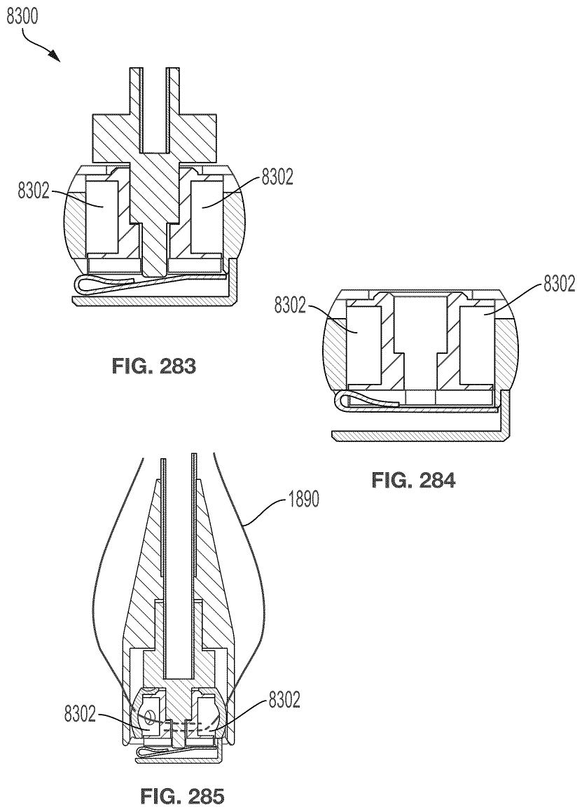

FIGS. 283-285 illustrate an example implementation of a width adjustment device 8300 that is configured to expand or contract the paddle frames of a device or implant. The width adjustment device 8300 can take any suitable form, such as, for example, any form described in the instant application. Moreover, any of the device or implants and width adjustment devices described herein can incorporate features of the width adjustment device 8300. In some implementations, the width adjustment device 8300 can be mechanically coupled to a distal cap, or to any other suitable attachment point described herein. In this way, it is appreciated that a wide variety of arrangements are contemplated.

In the illustrated example (FIGS. 283-285), the width adjustment device 8300 can include a spool mechanism 8302 for adjusting the width of the paddle frames. For example, a line can be secured to the paddle frames such that when the line is drawn (e.g., wound up) in by the spool mechanism (e.g., via a torque delivering tool), the line will pull on the paddle frames, thereby contracting the paddle frames.

Additional information regarding a spool mechanism and delivery method can be found in U.S. Patent Application Publication No. 2020/0113685 which is incorporated herein by reference in its entirety for all purposes. In addition, any of the width adjustment devices described herein can incorporate features of the spool mechanism 8302 and corresponding delivery method that is incorporated by reference herein. Moreover, it is appreciated that a variety of spool mechanisms can be used to expand or contract the paddle frames.

FIGS. 286-288 illustrate some implementations of a width adjustment device 8500 that is configured to expand or contract the paddle frames of a device or implant. The width adjustment device 8500 can take any suitable form, such as, for example, any form described in the present application. Moreover, any of the device or implants and width adjustment devices described herein can incorporate features of the width adjustment device 8500. The width adjustment device can include a line, such as a suture, wire, etc. that extends through eyelets 8402. In the illustrated example, paddle frames 8400 can include cam members 8404 (e.g., angled surfaces, tapered surfaces, rounded surfaces, etc.) that are configured to cooperate with the width adjustment device 8500 for biasing each arm 8406 of the paddle frame 8400 apart relative to each other. Referring to FIG. 287, when the width adjustment device 8500 is extended downwards, a protrusion, such as the illustrated wedge 8502 of the width adjustment device 8500 engages sloped surfaces of the cam members 8404 to push the cam members 8404 apart. Referring to FIG. 288, as the cam members 8404 are pushed apart as indicated by the arrows, each paddle frame arm 8406 pivots, flexes, and/or articulates outwards thereby increasing the width of the paddle frames 8400.

The width adjustment device 8500 can take a variety of different forms. In the example illustrated by FIG. 288, the width adjustment device includes a protrusion 8502 that is integrally formed with an externally threaded shaft 8505. The protrusion 8502 can take any suitable form. In the illustrated example, the threaded shaft 8505 is disposed in an internally threaded element or column 8508. When the shaft 8505 is driven into the cam members 8404, the protrusion 8502 pushes the cam members 8404 apart, thereby expanding the paddle frames 8400.

While the illustrated example depicts a threaded shaft 8505 for conveying the protrusion 8502, it should be understood that a wide variety of arrangements are contemplated. For example, a sliding or ratchet mechanism could be used to deploy the protrusion or wedge 8502.

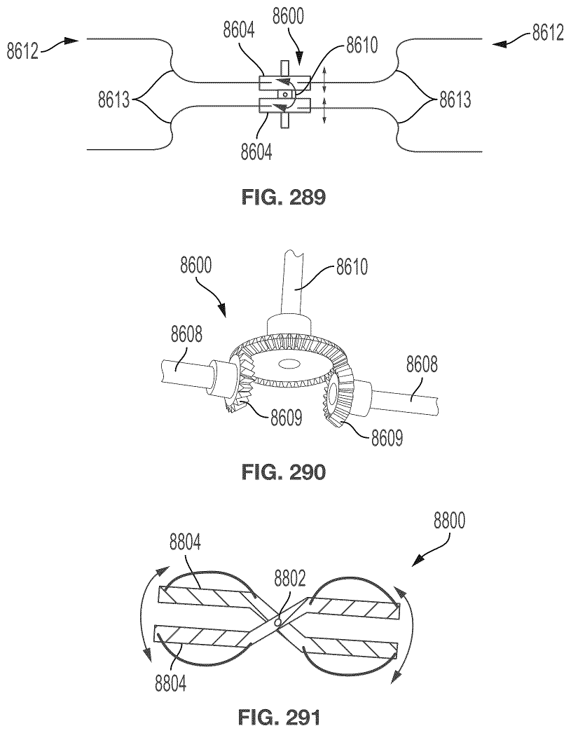

FIGS. 289 and 290 illustrate an example of a width adjustment device 8600 that is configured to expand or contract the paddle frames of a device or implant. The width adjustment device 8600 can take any suitable form, such as, for example, any form described in the present application. Moreover, any of the device or implants and width adjustment devices described herein can incorporate features of the width adjustment device 8600. In the illustrated example, the width adjustment device 8600 can include members 8604 (e.g., shafts, connectors, couplers, etc.) that are coupled to threaded shafts 8608, the shafts 8608 being connected to bevel gears 8609 (FIG. 290). A drive bevel gear 8610 could rotatably engage the driven bevel gears 8609. When the driven gears 8609 rotate, the members 8604 that are threadedly coupled to the threaded shafts 8608 move apart relative to each other. While the illustrated example depicts a right-angle bevel gear drive, is appreciated that a wide variety of mechanisms can be used to push the members 8604 and paddle frames 8612 apart relative to each other (e.g., a rack and pinion gear, a slider-crank mechanism, etc.).

Referring to FIG. 289, each member 8604 can be connected directly to two parallel paddle frames 8612 or be coupled to the two parallel paddle frames. In this manner, as the drive gear 8610 is rotated, it will cause the members 8604 that are coupled to the shafts 8608 to also move apart forcing struts 8613 of the paddle frames 8612 to move apart relative to each other. As the struts are moved apart, the paddle frames 8612 will begin to expand such that the width of the paddle frames 8612 is increased. Conversely, as the drive gear 8610 is rotated in the opposite direction, it will cause the members 8604 to move inwards relative to each other for contracting the paddle frames 8612, respectively.

Referring to FIG. 291, an example implementation of a width adjustment device, such as the illustrated scissor mechanism 8800 is shown. In the illustrated example, the scissor mechanism 8800 is configured to expand or contract paddle frames 8804 that are pivotally attached to a center axis or shaft 8802 of the scissor mechanism 8800. The scissor mechanism 8800 can take any suitable form. Any of the device or implants described herein can incorporate features of the scissor mechanism 8800. A width adjustment device (e.g., any suitable device described herein) can be coupled to the paddle frames 8804, such that when the width adjustment device is activated, it will cause the paddle frames 8804 to pivot upon the shaft 8802 and move outwards relative to each other thereby increasing the width of the paddle frames 8804. The width adjustment device is also be configured to cause the paddle frames 8804 to contract.

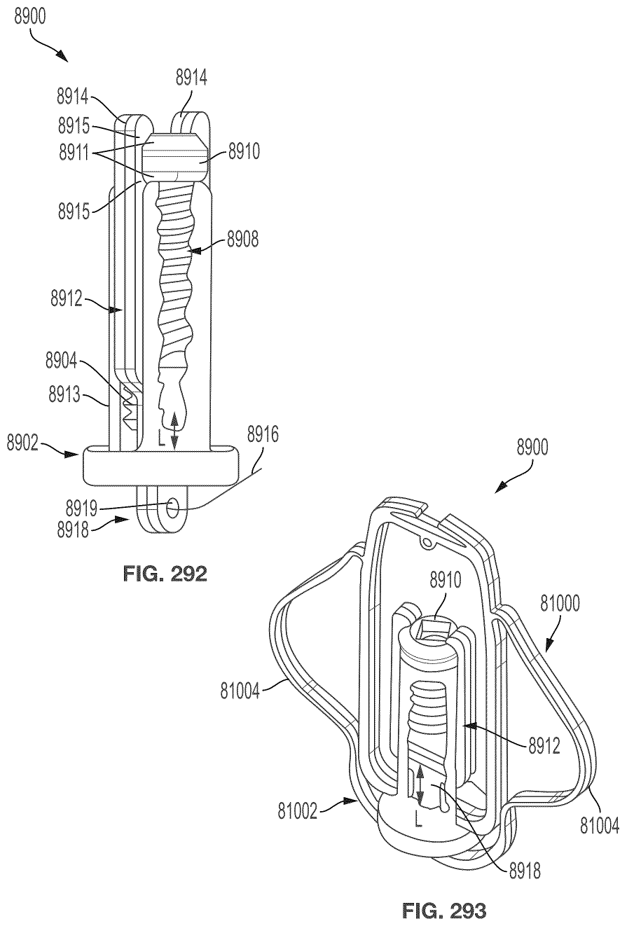

FIG. 292 illustrates an example of a width adjustment and actuation device 8900 that is configured to expand or contract the paddle frames of a device or implant and to open and close the paddle frames. The width adjustment and actuation device 8900 can take any suitable form, such as, for example, any form described in the present application. Moreover, any of the device or implants and width adjustment devices described herein can incorporate features of the width adjustment and actuation device 8900. In the illustrated example, the width adjustment and actuation device 8900 includes a coupler, such as the illustrated shaft 8908, and a receiver (e.g., an internally threaded element, a notched receiving portion, a column, a lumen, a tube, a shaft, a sleeve, a post, a housing, tracks, a cylinder, etc.), such as the illustrated housing 8902. In some implementations, the housing 8902 can be an integral part of a device or implant. For example, the housing 8902 can be integrally formed with the distal cap or any other suitable member described herein. The shaft 8908 includes an external thread pattern that is configured to threadedly engage a female thread pattern 8904 formed in the housing 8902. A driver head 8910 is integrally formed at a proximal end of the shaft 8908 and is configured to enable rotation of the shaft 8908 by a variety of tools or drive types (e.g., Torx, slotted, Philips, etc.).

Still referring to FIG. 292, a receiver, such as the illustrated fork-shaped carriage 8912, is disposed around the shaft 8908 and the driver head 8910. In the illustrated example, the carriage 8912 features proximal tines 8914 and a distal end 8918 which are formed as a single, unitary component. However, it is understood that the carriage 8912 can take any suitable form, such as, for example, any form described in the present application.

Still referring to FIG. 292, the driver head 8910 features mating surfaces 8911 that are configured to be complementary with surfaces 8915 of the proximal tines 8914, respectfully, for harnessing the carriage 8912 to the driver head 8910. Torque prevention cutaways 8913 formed in the housing 8902 are configured to receive and constrain the carriage 8912 to longitudinal movement in the direction L and prevent or inhibit the carriage 8912 from rotating when torque is applied to the driver head 8910. Therefore, when the driver head 8910 is rotated, the driver head 8910 will pull the carriage 8912 such that the carriage 8912 is confined to move in an upward or downward direction along a longitudinal axis of the shaft 8108 as indicated by arrows L.

Still referring to FIG. 292, the distal end 8918 of the carriage 8912 is formed with an aperture 8919 that is configured to permit a line 1890 to pass therethrough. Opposite ends of the line 1890 can be secured to various attachment points on the paddle frames, the paddles, the distal cap, or to any other suitable attachment point described herein. When the driver head 8910 is rotatably driven to move the carriage 8912, the distal end 8918 of the carriage 8912 will pull on the line 8916, thereby causing the paddle frames (not shown) to contract. In the illustrated example, rotating the driver head 8910 clockwise (right-handed thread configuration) will cause the carriage 8912 to move downwards along the longitudinal axis of the shaft 8908 in the direction illustrated by arrows L. In this way, the distal end 8918 of the carriage 8912 will pull on the line 1890 causing the paddle frames to contract. However, it is understood that other configurations are also contemplated. For example, rotating the driver head 8910 in a counterclockwise direction could apply tension to the line 1890 for causing the paddle frames to contract. Moreover, it is appreciated that in other configurations, applying tension to the line 1890 could cause the paddle frames to expand, rather than contract.

Referring to FIG. 293, an example of a width adjustment and actuation device 8900 and a retractable/expandable paddle frame is shown. In the illustrated example, the width adjustment and actuation device 8900 of FIG. 293 is substantially the same as that of the example shown in FIG. 292, except that the distal end 8918 of the carriage 8912 is integrally formed with a distal end 81002 of paddle frames 81000 of a device or implant. However, it should be understood that in some implementations, the carriage 8912 can be integrally formed with the distal cap, or with any other suitable member described herein.

Still referring to FIG. 293, when the driver head 8910 is rotatably driven to convey the carriage 8912, the carriage 8912 will cause a distal portion 81002 to move upward or downward. Upward movement of the distal portion 81002 causes the paddle frames 81000 to flex outward or expand and downward movement of the distal portion 81002 causes the paddle frames 81000 of the paddle frames 81000 to flex inward or retract. For example, when rotating the driver head 8910 clockwise (right-handed thread configuration), the carriage 8912 will move in a downward direction along the longitudinal axis causing the distal portion 81002 of the paddle frames 81000 to move downward, and lateral portions 81004 of the paddle frames 81000 to contract inward, thereby reducing the overall width of the paddle frames 81000. When rotating the driver head 8910 counterclockwise (right-handed thread configuration), the carriage 8912 will move in an upward direction along the longitudinal axis causing the distal portion 81002 of the paddle frames 81000 to move upward, and lateral portions 81004 of the paddle frames 81000 to expand outward, thereby increasing the overall width of the paddle frames 81000.

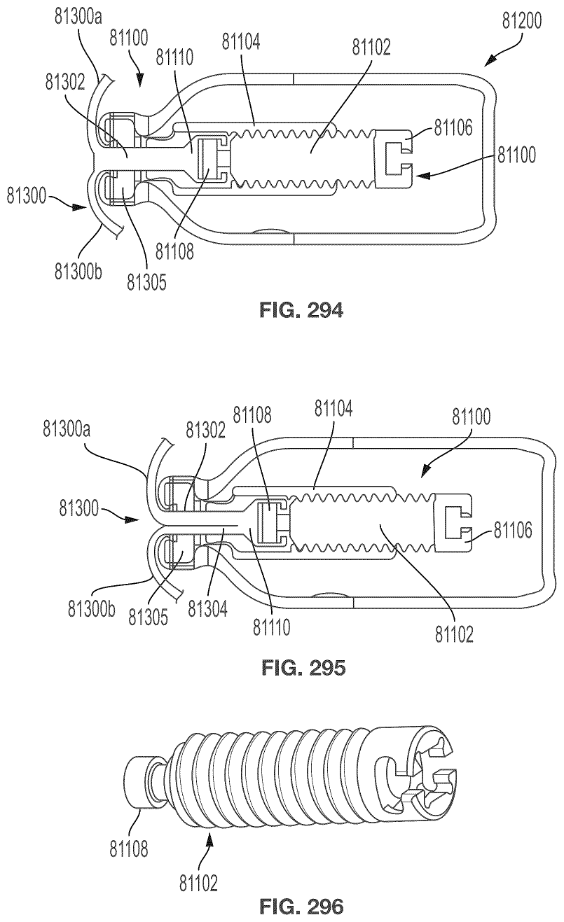

FIG. 294 illustrates an example of a width adjustment and actuation device 81100 that is configured to expand or contract the paddles of a device or implant 81200. The width adjustment and actuation device 81100 can take any suitable form, such as, for example, any form described in the present application. Moreover, any of the device or implants and width adjustment devices described herein can incorporate features of the width adjustment and actuation device 81100. In the illustrated example, the width adjustment and actuation device 81100 includes a coupler, such as the illustrated externally threaded shaft 81102 (FIG. 296) that is rotatably engaged with a receiver (e.g., an internally threaded element, a notched receiving portion, a column, a lumen, a tube, a shaft, a sleeve, a post, a housing, tracks, a cylinder, etc.), such as the internally threaded element 81104 (illustrated and often referred to as a “column” herein, but can be or comprise other types of threaded elements and have a variety of different sizes and shapes as well) that is integrally formed with a distal portion of a device or implant. For example, the threaded element or column 81104 can be integrally formed with the distal cap, a distal portion of the paddle assembly, or with any other suitable member described in the present application.

A driver head 81106 is disposed at a proximal end of the shaft 81102 and is configured to rotatably drive the shaft 81102 into or out of the threaded element or column 81104. The driver head 81106 can take any form, such as for example, any form described in the present application. Referring to FIG. 296, a coupler 81108 is attached to a distal end of the shaft 81102 and is configured to be retained by a receiver 81110 (FIG. 294) that is formed on an inner end or post member 81302 (e.g., post, shaft, etc.). The inner end or post member 81302 is configured to mechanically couple the expandable/retractable paddle frames 81300 to the coupler 81108. In this way, when the driver head 81106 is driven to rotate the shaft 81102 counterclockwise (e.g., right-handed thread configuration), the shaft 81102 will rotate and move toward a proximal end of the width adjustment and actuation device 81100 causing the coupler 81108 to pull on the receiver 81110 of the paddle frames 81300. As the receiver 81110 is pulled by the coupler 81108, the paddle frames 81300 will begin to contract inward and reduce the overall width of the paddle frames 81300. Conversely, rotating the shaft 81102 clockwise (e.g., into the threaded element or column 81104) will cause the paddle frames 81300 to expand outwards. However, it should be understood that other configurations are also contemplated. For instance, in some implementations, rotating the shaft 81102 clockwise will cause the paddle frames 81300 to contract inwards. Therefore, it is appreciated that a wide variety of configurations are contemplated for expanding or contracting the paddle frames.

FIG. 295 illustrates an example of a width adjustment and actuation device 81100 that is configured to expand or contract the paddles of a device or implant is shown. The width adjustment and actuation device 81100 can take any suitable form, such as, for example, any form described in the present application. Moreover, any of the device or implants and width adjustment devices described herein can incorporate features of the width adjustment and actuation device 81100. In the illustrated example, the width adjustment and actuation device 81100 of FIG. 295 is substantially the same as the example shown in FIG. 294, except that the inner end or post 81302 is partially split along partition line 81304. The split inner end or post 81302 connects the coupler 81108 to the paddle frames 81300. The paddle frames 81300 are partially retractable into a distal portion 81305 (e.g., distal cap) of a device or implant. In particular, sheathable portions 81300a and 81300b of the paddle frames 81300 can be drawn in and through the distal portion 81305 and into the cavity that is formed by the internally threaded column 81104. In this way, when the driver head 81106 causes the coupler to pull on the receiver 81110 for contracting the paddle frames 81300 and drawing the sheathable portions 81300a 81300b in through the distal portion 81305. The contracting paddle frames are particularly advantageous when having to navigate a device or implant through tight spaces, such as through the chordae tendineae (e.g., such as, when deploying the device).

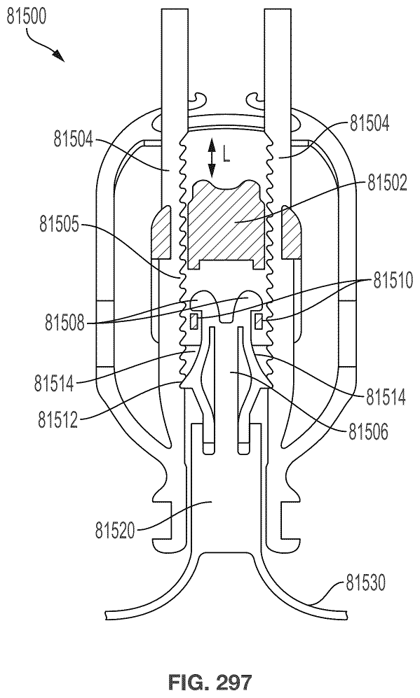

Referring to FIG. 297, an example implementation of a width adjustment device is shown. Any of the device or implants and width adjustment devices described herein can incorporate features of a width adjustment and actuation device 81500. In the illustrated example, the width adjustment and actuation device 81500 includes a coupler, such as the illustrated push/pull member 81502 (e.g., coupler, shuttle, slider, etc.), a receiver (e.g., an internally threaded element, a notched receiving portion, a column, a lumen, a tube, a shaft, a sleeve, a post, a housing, tracks, a cylinder, etc.), such as the illustrated parallel racks 81504, and a coupling member 81506 (e.g., flexible arms, etc.). Each rack 81504 includes teeth 81505 that are configured to limit the motion of the coupling member 81506 to a single direction (e.g., a ratchet mechanism) when the coupling member is in an engaged state. In the illustrated example, the coupling member 81506 is coupled to paddle frames 81530 by an inner end or connection portion 81520. In some implementations, the coupling member 81506, the inner end or connection portion 81520, and the paddle frames 81530 can be formed as a single, unitary component.

Still referring to FIG. 297, arms 81508 are formed on the coupling member 81506 and are configured to engage projections 81510 of the actuator or push/pull member 81502 (e.g., see sectional view of FIG. 297). Resilient fingers 81512 are also formed on the coupling member 81506 and are configured to engage the teeth 81505 of the rack 81504 for preventing or inhibiting the coupling member 81506 from moving along the path L in a downward or distal direction of the racks 81504.

Still referring to FIG. 297, the actuator or push/pull member 81502 can be driven in the directions indicated by arrows L. When the actuator or push/pull member 81502 is driven upwards, the projections 81510 of the actuator or push/pull member 81502 will pull the coupling member 81506 via the arms 81508 of the coupling member 81506. As a result, the resilient fingers 81512 will ratchet along the teeth 81505 of the rack 81504, thereby permitting the coupling member 81506 to move upwards when the actuator or push/pull member 81502 moves upwards. Simultaneously, the coupling member 81506 will cause the inner end or connection portion 81520 to pull on the paddle frames 81530 and cause the paddle frames 81530 to contract. In such implementations, the position of the resilient fingers 81512 relative to each of the plurality of discrete positions (i.e., the teeth) on the rack 81504 can correspond to a particular width of the paddle frames, respectively.

Conversely, when the actuator or push/pull member 81502 is driven downwards, the projections 81510 of the actuator or push/pull member 81502 push against resilient, sloped surfaces 81514 of the coupling member 81506. As such, the projections 81510 cause the resilient fingers 81512 to disengage from the rack 81504. As such, the coupling member 81506 is disengaged from the rack 81504 when the actuator is moved in a downward or distal direction to expand the paddle frames 81530.

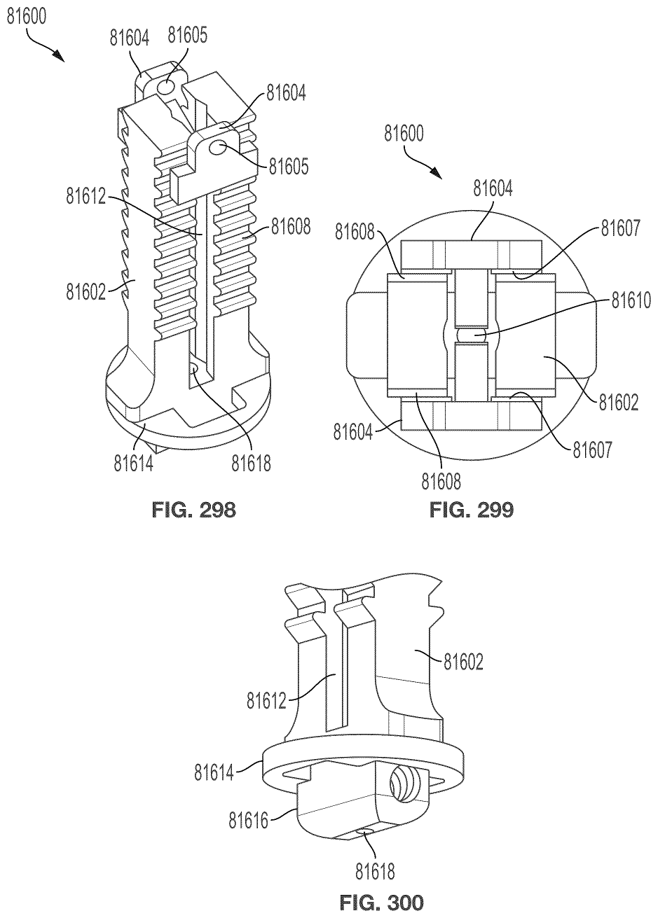

FIG. 298 illustrates an example of a width adjustment device 81600 that is configured to expand or contract the paddles of a device or implant. Any of the device or implants and width adjustment devices described herein can incorporate features of the width adjustment device 81600. Referring to FIGS. 298 and 299, the width adjustment device 81600 includes a central post 81602 and two coupling members 81604 that are releasably coupled to the central post 81602. In the illustrated example, the coupling members 81604 are biased towards the central post 81602 by way of a biasing means, such as, for example, a spring (e.g., coil, leaf, torsion) or spring-like material, such as steel and/or shape-memory alloys such as Nitinol, and the like. However, the biasing means can take any suitable form, such as, for example, any form described in the present application.

Still referring to FIG. 298, the coupling members 81604 have gripping pawls 81607 that are configured to latch onto teeth 81608 formed on the central post 81602. The biasing means forces the gripping pawls of each coupling member 81604 to latch onto the teeth 81608 formed on the central post 81602 for securing the coupling members 81604 to the central post 81602.

To disengage the coupling members 81604 from the central post 81602, a driver element (e.g., wire, shaft, etc., not shown) can be inserted into a receiving bore 81610 (FIG. 299) formed in the central post 81602. As the driver wire is inserted into the bore 81610, the driver wire will push both coupling members 81604 outwards relative to each other against the force of the biasing means. In this manner, the disengaged coupling members 81604 become slidable along a track 81612 that is formed in the central post 81602.

Still referring to FIG. 298, an optional flange 81614 can be formed at a distal end of the central post 81602. In some implementations, the distal flange 81614 is integrally formed with a distal portion a device or implant. For example, the distal flange 81614 can be formed with the distal cap, a distal portion of the paddle frames, or with any other suitable member described in the present application.

Referring to FIG. 300, the flange 81614 can optionally include a downwardly extending boss 81616. In the illustrated example, a cavity 81618 is formed in the boss 81616 and that can serve as an entry point for actuation element (not shown) that is used to decouple the coupling members 81604 (e.g., pawls, etc.) to the paddle frames and/or to open and close the device. Control elements, (e.g., wires, shafts, lines, etc.) which can be separate from the actuation element, are releasably attached to the apertures 81605 of the coupling members 81604. When the driver wire is inserted into the receiving bore 81610 to decouple the coupling members 81604 from the central posts, the control wires can move the coupling members along the central post 81602, either independently or in unison. The resulting movement of the coupling members 81604 can impose a tension on a width adjustment line. For example, when the coupling members 81604 are moved up the central post 81602, tension will be applied to lines causing the paddle frames to contract. Conversely, moving the coupling members 81604 down the central post 81602 will decrease tension applied to the line and allow the paddle frames to expand outwards. However, it should be understood that in other configurations, moving the coupling members 81604 up the central post 81602 will cause the paddle frames to expand and moving the coupling members 81604 down the central post 81602 will cause the paddle frames to contract.

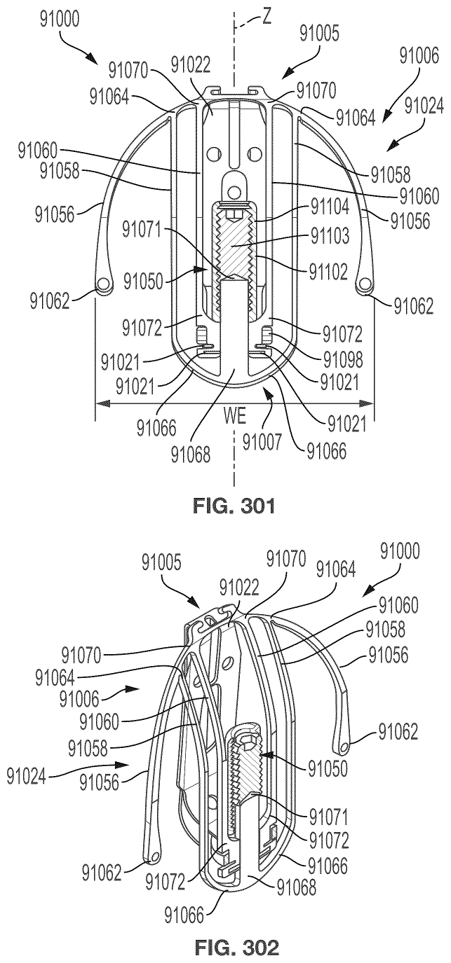

Referring to FIGS. 301-302, an example implementation of a device or implant 91000 is shown. The device or implant 91000 includes a proximal or attachment portion 91005, anchor portions 91006 that include paddle frames 91024, a width adjustment and actuation device 91050, and a distal portion 91007. The paddle frames 91024 have a height H (FIG. 304) between the proximal portion 91005 and the distal portion 91007. The anchor portion 91006 includes inner paddles 91022 and outer paddles 91020. The attachment portion 91005, the distal portion 91007, the anchor portion 91006, and the width adjustment and actuation device 91050 can be configured in a variety of ways.

The paddle frames 91024 are configured to allow the device 91000 to maneuver more easily into position for implantation in the heart by reducing the contact and/or friction between the native structures of the heart—e.g., chordae—and the device. That is, the paddle frames 91024 are configured to move between an expanded condition and a narrowed condition. When the paddle frames 91024 are in the narrowed condition, the contact between the native structures of the heart and the device 91000 is reduced. The device 91000 can include any other features of devices or implants discussed elsewhere in the present application or in the applications and patents incorporated by reference herein, and the device 91000 can be positioned to engage valve tissue 20, 22 as part of any suitable valve repair system (e.g., any valve repair system disclosed in the present application). In addition, any of the devices described herein can incorporate the features of the device 91000.

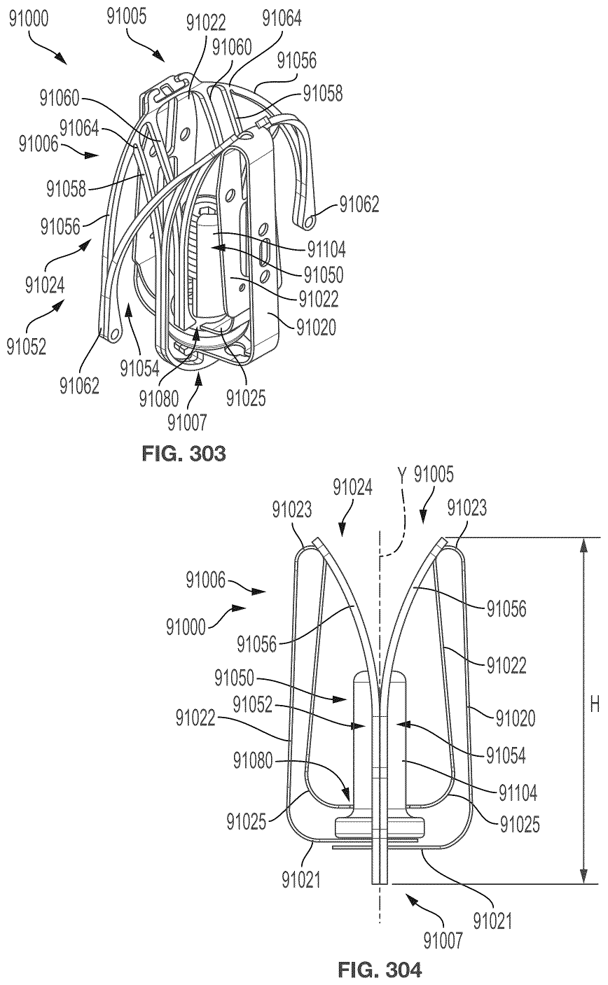

In the illustrated example of FIGS. 301-317, the paddle frames 91024 are symmetric along longitudinal plane Y (FIG. 304) and are symmetric along longitudinal plane Z (FIG. 301). In some implementations of the device or implant 91000, however, the paddle frames 91024 is not symmetric about one or both of the planes Y and Z. The paddle frames 91024 include a first frame side 91052 and a second frame side 91054 that is a mirror image of the first frame side 91052 (FIGS. 303-317).

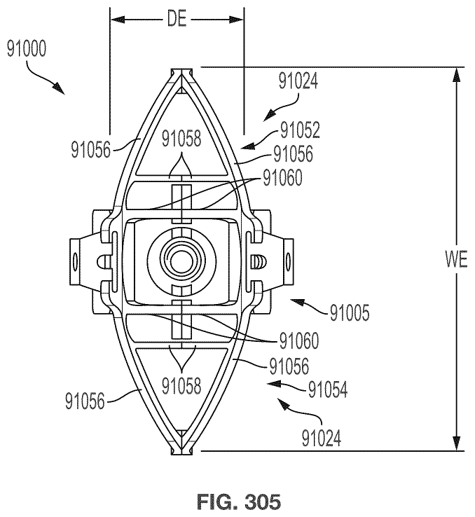

In the illustrated example in FIGS. 301-305, the paddle frames 91024 includes outer frame members 91056 (e.g., arms, etc.), intermediate frame members 91058 (e.g., struts, etc.), and inner frame members 91060 (e.g., struts, etc.). In FIG. 301, the outer frame members 91056 are shown in an expanded state such that the outer frame members 91056 define a paddle frame width WE in the expanded state and a paddle frame depth DE in the expanded state (FIG. 305). The outer frame members 91056 are attached to the intermediate frame members 91058 at the proximal portion 91005 and include terminal distal ends 91062. The outer frame members 91056 are curved and can form a semicircle or U-shape. In some implementations, however, the outer frame members 91056 can be otherwise shaped.

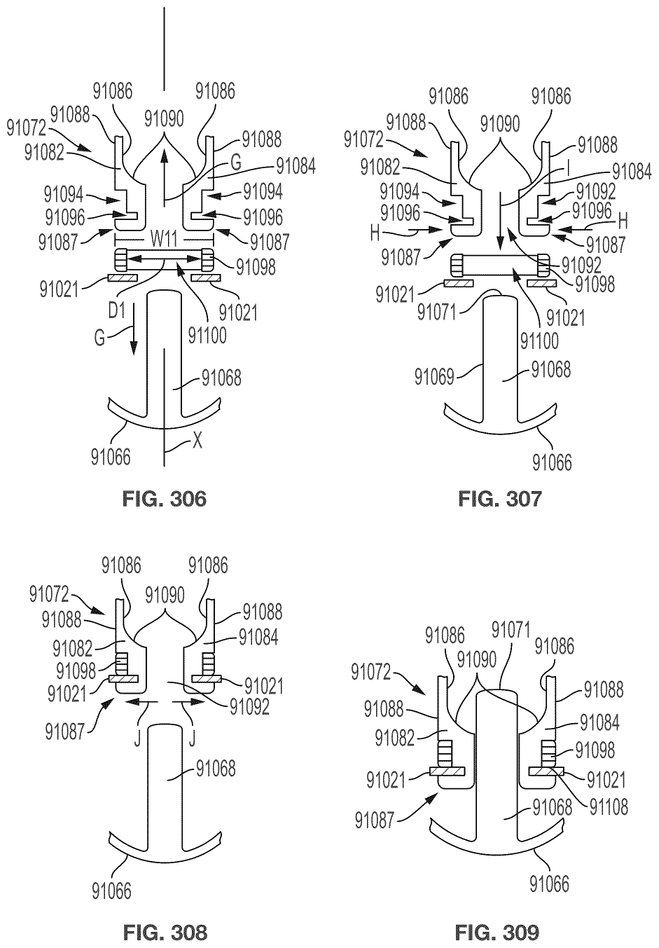

The intermediate frame members 91058 extend from a connection portion 91064 with the outer frame members 91056 near or at the proximal portion 91005 and are attached at the distal portion 91007 via connection portions 91066. The intermediate frame members 91058 also include an inner end, which in the illustrated example is configured as a projection or post 91068 (FIGS. 301 and 302) extending axially along axis Z from the distal portion 91007 toward the proximal portion 91005. The inner end or post 91068 can be configured in a variety of ways. In the illustrated example, the inner end or post 91068 has a cylindrical outer side surface 91069 and an end surface 91071 perpendicular to the outer side surface (see FIG. 306).

The inner frame members 91060 extend from a connection portion 91070 with the outer frame members 91056 near or at the proximal portion 91005 and include retaining portions 91072 near or adjacent the distal portion 91007 for engaging the inner end or post 91068. The retaining portions 91072 are described below in more detail with regard to FIGS. 306-311.

The first frame side 91052 and a second frame side 91054 can optionally be in contact with each other along axis Y toward the distal end 91007 and are separated toward the proximal end 91005 to form a V-shape, as shown, for example, in FIG. 303.

Referring to FIGS. 302-304, the outer paddles 91020 are connected to the retaining portions 91072 at the distal end 91007 via connection portions 91021 and to the inner paddles 91022 by connection portions 91023. The inner paddles 91022 are connected to a coaptation portion or inner member (not shown) by connection portions 91025. Referring to FIGS. 302-304, the inner paddles 91022 are not connected to the retaining portions 91072. Instead, the inner paddles 91022 form an aperture or gap 91080 through which the retaining portions 91072 extend.

Referring to FIGS. 306-311, the method of assembling the retaining portions 91072 to the inner end or post 91068 is illustrated. The retaining portions 91072 include a first retaining portion 91082 and a second retaining portion 91084 spaced apart from, and a mirror image of, the first retaining portion 91082. Each of the inner frame members 91060 and retaining portions 91072 includes an inner side surface 91086, an outer side surface 91088 opposite the inner side surface 91086, and a distal end 91087.

The inner side surfaces 91086 of the inner frame members 91060 include inward transition portions 91090 that form a seat. In the illustrated example, the inward transition portions 91090 are formed as inward curved surfaces. In some implementations, however, the inward transition portions 91090 can be formed in any suitable manner, such as for example, as angled or tapered surfaces, stepped surfaces, or any other suitable inward transition. The inner side surfaces 91086 extend axially from the inward transition portion 91090 toward the distal end 91007 to form a gap 91092 configured to receive the inner end or post 91068.

Each of the outer side surfaces 91088 of the inner frame members 91060 includes a first recessed portion 91094. In the illustrated example, the first recessed portion 91094 is formed axially nearer to the distal end 91007 than the inward transition portion 91090 is located. Each of the first recessed portions 91094 include a second recessed portion 91096 that is recessed relative to the first recessed portion 91094. In the illustrated example, the second recessed portion 91096 is located at a portion of the first recessed portion 91094 that is closest to the distal portion 91007. The second recessed portions 91096 are configured to receive the connecting portions 91021 of the outer paddles 91020.

The first recessed portions 91094 are configured to receive an annular retainer 91098. The annular retainer 91098 can be a ring, a washer, a nut, or the like. The annular retainer 91098 includes an inner passage 91100 configured to receive the inner end or post 91068 therethrough. The inner passage 91100 has a diameter D1 which is less than the combined width W11 of the distal ends 91087 and gap 91092 in an uncompressed state as shown in FIG. 306.

FIG. 301 illustrates an assembled state for the device 91000 in which the inner end or post 91068 is received through the gap 91092 in the retaining portion 91072. To assemble the device 91000, as shown in FIG. 306, the inner end or post 91068 and connecting portion 91066 of the intermediate frame members 91058 and the retaining portion 91072 of the inner frame members 91060 are pulled away from each other along the plane X, as shown by arrow G.

The paddle frames 91024 can be made from a material that allows the inner end or post 91068 and connecting portion 91066 of the intermediate frame members 91058 and the retaining portion 91072 of the inner frame members 91060 to be pulled away from each other. For example, the paddle frames 91024, or a portion thereof, can be made of a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, some portions of the paddle frames 91024 can be stiffer or more rigid than other portions. For example, in the illustrated example of the paddle frames 91024, the inner frame members 91060 can be configured to be stiffer than the outer frame members 91056. The inner frame members 91060 can be configured in a variety of ways to be stiffer or more rigid. For example, the thickness of the inner frame members 91060 and/or the material used for the inner frame members 91060 can provide more rigidity. In some implementations, the thickness of the inner frame members 91060 can be greater than the outer frame members 91056 to provide more rigidity. Further, in some implementations, the material used in the inner frame members 91060 can be a more rigid material to provide more rigidity.

Once the inner end or post 91068 and the connecting portion 91066 are separated from the retaining portion 91072, the annular retainer 91098 and the connecting portions 91021 of the outer paddles 91020 can be placed therebetween. As shown by arrows H in FIG. 307, the distal ends 91087 of the first retaining portion 91082 and a second retaining portion 91084 can be compressed toward each other such that the gap 91092 is reduced or closed. The distal ends 91087 can be compressed such that the combined width of the distal ends 91087 and the gap 91092 is less than the diameter D1 of the passage 91100 of the annular retainer 91098. As such, the distal ends 91087 can be received through the passage 91100 and between the connection portions 91021 of the outer paddles 91020, as shown by arrow I in FIG. 307

As shown in FIGS. 308, once the distal ends 91087 are received through the passage 91100 and between the connection portions 91021 of the outer paddles 91020, the annular retainer 91098 can be aligned with the first recessed portions 91094 and the connecting portion 91021 of the outer paddles 91020 can be aligned with the second recessed portions 91096. The distal ends 91087 can then be released to return toward the uncompressed state, as shown by arrows J, while the annular retainer 91098 is received in the first recessed portions 91094 and the connecting portions 91021 of the outer paddles 91020 are received with the second recessed portions 91096.

The distal ends 91087 can be configured to provide an outward bias on the annular retainer and/or the connecting portions 91021 of the outer paddles 91020 to provide a secure attachment between the annular retainer and/or the connecting portions 91021 of the outer paddles 91020. As shown in FIG. 309, once the annular retainer 91098 is received in the first recessed portions 91094 and the connecting portions 91021 of the outer paddles 91020 are received with the second recessed portions 91096, the inner end or post 91068 and the connecting portions 91066 of the intermediate frame members 91058 can be released, which allows the inner end or post 91068 to be received through the gap 91092 and the end surface 91071 extends past the inward transition portions 91090.

FIGS. 306-311 illustrate two connecting portions 91021 of the outer paddles 91020. For example, the outer paddles 91020 can be jointably attached at a distal portion 91007, such as for example, similar to the outer paddles 9320 of FIGS. 380-381. FIGS. 301-302 and 927, however, shown the connecting portions 91021 of the outer paddles 91020 not be jointly attached and being offset. Thus, the retaining portions 91072 can include an additional recessed portion (not shown) to receive one of the offset retaining portions 91072. The additional recessed portion (not shown) can be provided on the inner surface 91086 or on the outer surface 91088 of the retaining portion 91072.

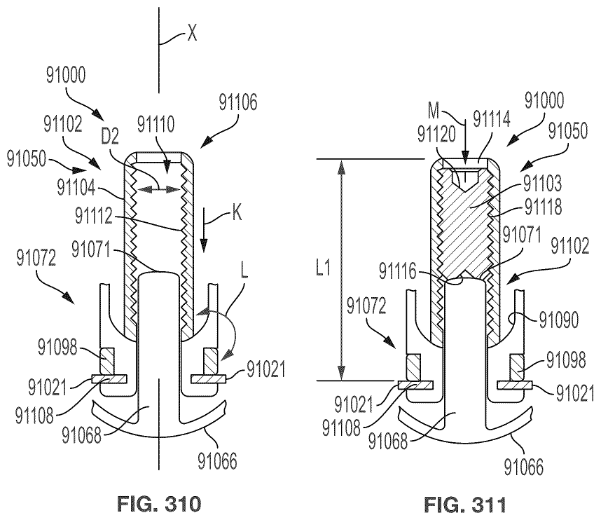

Referring to FIG. 310, the width adjustment and actuation device 91050 of device or implant 91000 is configured to both facilitate moving the paddle frames 91024 between an expanded position and a narrowed position and move the paddles of the device 91000 between a closed position and an open position. The width adjustment and actuation device 91050 can be configured in a variety of ways. Any structure capable of selectively moving the paddle frames 91024 between an expanded position and a narrowed position and moving the paddles of the device between an open position and a closed position can be used. In some implementations, the width adjustment and actuation device are configured such that advancing and retracting the width adjustment and actuation device itself opens and closes the device and advancing and retracting the post inside the width adjustment and actuation device narrows and widens the paddles. For example, the stiffer inner paddle frame portions are moved by the width adjustment and actuation device to open and close the paddles in the same or a similar manner to that shown in FIGS. 23, 27, and 30-37.

In the illustrated example, the width adjustment and actuation device 91050 includes a receiver (e.g., an internally threaded element, a notched receiving portion, a column, a lumen, a tube, a shaft, a sleeve, a post, a housing, tracks, a cylinder, etc.) such as the illustrated sleeve 91102 configured to receive a portion of the inner end or post 91068 and a coupler, such as the illustrated plug 91103, configured to move the post axially within the sleeve 91102. During assembly, the sleeve 91102 can be received onto the inner end or post 91068 as shown by arrow K.

The sleeve 91102 can be configured in a variety of ways. In the illustrated example, the sleeve 91102 includes a cylindrical sidewall 91104 extending between a proximal end 91106 to a distal end 91108 of the annular retainer 91098. The sleeve 91102 can optionally be integrally formed with the annular retainer 91098. The sleeve 91102 defines an internal passage 91110.

The sleeve 91102 has a length L1 and the internal passage 91110 extends through the entire length L1 of the sleeve 91102 from the proximal end 91106 and a distal end 91108. The passage 91110 has a diameter D2 that is sufficient to allow the inner end or post 91068 to be received into the passage 91110 and includes an internal threaded portion 91112.

As shown by arrow L in FIG. 310, the distal end 91108 of the sleeve 91102 is fixedly attached to the retaining portion 91072. The sleeve 91102 can be attached to the retaining portion 91072 in any suitable manner. In the illustrated example, annular retainer 91098 at the distal end 91108 is attached to the recess 91096 of the retaining portion 91072. The passage 91110 is aligned with the gap 91092 such that the inner end or post 91068 extends through the gap 91092 and into the passage 91110.

As shown FIG. 311, the plug 91103 is received within the passage 91110. The plug 91103 is configured to move axially within the sleeve 91102, as shown by arrow M. In the illustrated example, the plug 91103 is cylindrical and includes a proximal end 91114, a distal end 91116 opposite the proximal end, and an external threaded portion 91118. The external threaded portion 91118 is configured to threadedly engage with the internal threaded portion 91112 of the sleeve 91102.

The proximal end 91114 includes a drive interface 91120 configured to engage a drive member capable of rotating the plug 91103 to axially move the plug 91103 relative to the sleeve 91102. The drive interface 91120 can be any suitable interface. For example, the drive interface 91120 can be a drive recess, such as a slotted, hexagonal, Torx, Frearson, Phillips, square, or other suitable interface. The distal end 91116 forms an engagement surface configured to engage the proximal end 91071 of the inner end or post 91068.

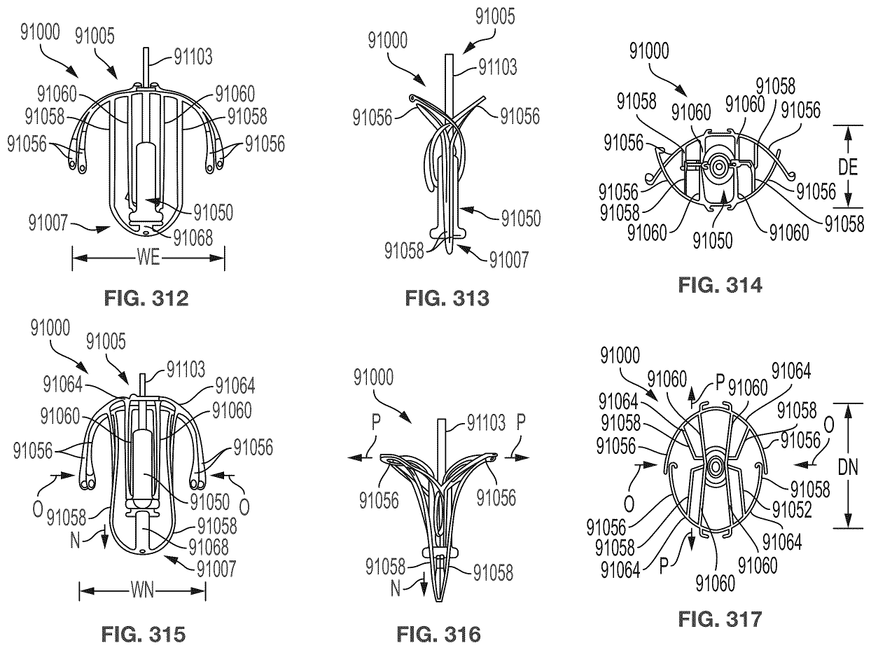

As shown in FIGS. 312-314, in the expanded state, the majority of, or most of, the inner end or post 91068 is received within the width adjustment and actuation device 91050 and the device 91000 has the width WE defined by the positions of the outer frame members 91056 and the depth DE. The plug 91103 is illustrated as extending out of the width adjustment and actuation device 91050 toward the proximal portion 91005 of the device 91000. However, in some implementations, the plug does not extend past the proximal end of the width adjustment and actuation device 91050 and an actuation rod is coupled to the plug 91103 in the width adjustment and actuation device 91050 or at the end of the receiver.

As shown in FIG. 314, for the illustrated example, in the expanded state, the top view of the device 91000 has the shape of a lens (i.e., a convex region bounded by two circular arcs that intersect at, or near, their endpoints).

Referring to FIGS. 310-311, in operation, to move the device 91000 from the expanded position to the narrowed position, the plug 91103 can be rotated via the drive interface 91120 to move the plug 91103 axially relative to the sleeve 91102. Movement of the plug toward the distal end 91108 of the sleeve 91102 causes the distal end 91116 of the plug 91103 to engage the proximal end 91071 of the inner end or post 91068 and move the inner end or post 91068 in the same direction (i.e., away from the proximal portion 91005 of the device).

As shown in FIGS. 315-317, movement of the inner end or post 91068 away from the proximal portion 91005 pulls the intermediate frame members 91058 in the same direction, as shown by arrow N in FIGS. 315 and 316. Due to the connection of the intermediate frame members 91058 to the outer frame members 91056 at the connection portions 91064, movement of the intermediate frame members 91058 away from the proximal portion 91005 pulls the outer frame members 91056 inward (i.e., to the narrowed position), as shown by arrows O in FIGS. 315 and 317, such that the device 91000 has a width WN in the narrowed position that is narrower than the width WE in the expanded position.

When the device 91000 narrows in width to the narrowed position, the device 91000 can also widen in the depth dimension, as shown by arrows P in FIGS. 316 and 317. As shown in FIG. 317, in the narrowed position, the device 91000 has a depth DN which is greater than the depth DE in the expanded position. In addition, the top view of the device 91000 changes from a lens shape, in the expanded position, to a circular or oval shape in the narrowed position, as shown in FIG. 317. Thus, the paddle frames 91024 can be moved between an expanded position and a narrowed position by rotating the plug 91103 within the sleeve 91102.

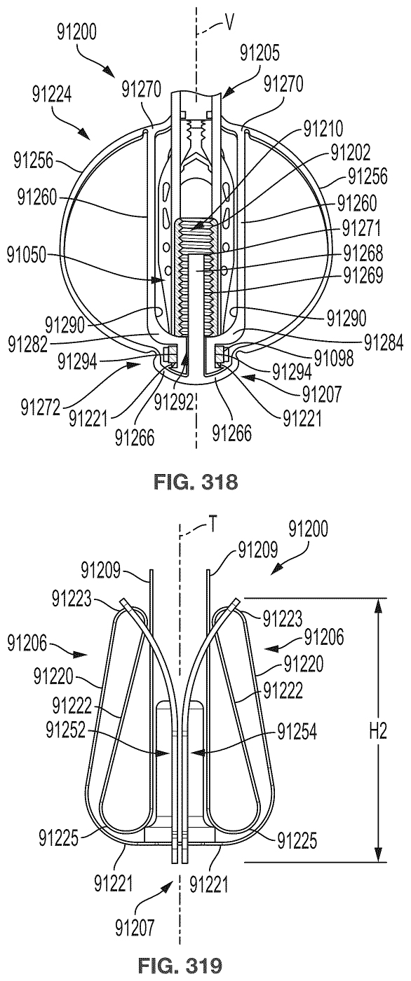

Referring to FIGS. 318-323, an example implementation of a device or implant 91200 is shown. The device or implant 91200 includes a proximal or attachment portion 91205, an anchor portion 91206 (FIG. 319), paddle frames 91224, a width adjustment and actuation device 91050, and a distal portion 91207. The paddle frames 91224 have a height H2 (FIG. 319) between the proximal portion 91205 and the distal portion 91207. Referring to FIG. 319, the anchor portion 91206 includes inner members 91209 (e.g., posts, elongated sheet material portions, etc.), inner paddles 91222, and outer paddles 91220. The attachment portion 91205, the distal portion 91207, the anchor portion 91206, the width adjustment and actuation device 91050, and the paddle frames 91224 can be configured in a variety of ways.

In the illustrated example of FIGS. 318-319, the paddle frames 91224 are symmetric along longitudinal axis T (FIG. 319) and are symmetric along longitudinal axis V (FIG. 318). In some implementations of the device or implant 91200, however, the paddle frames 91224 are not symmetric about one or both of the axes T and V. The paddle frames 91224 include a first frame side 91252 and a second frame side 91254 that is a mirror image of the first side 91252 (FIG. 319).

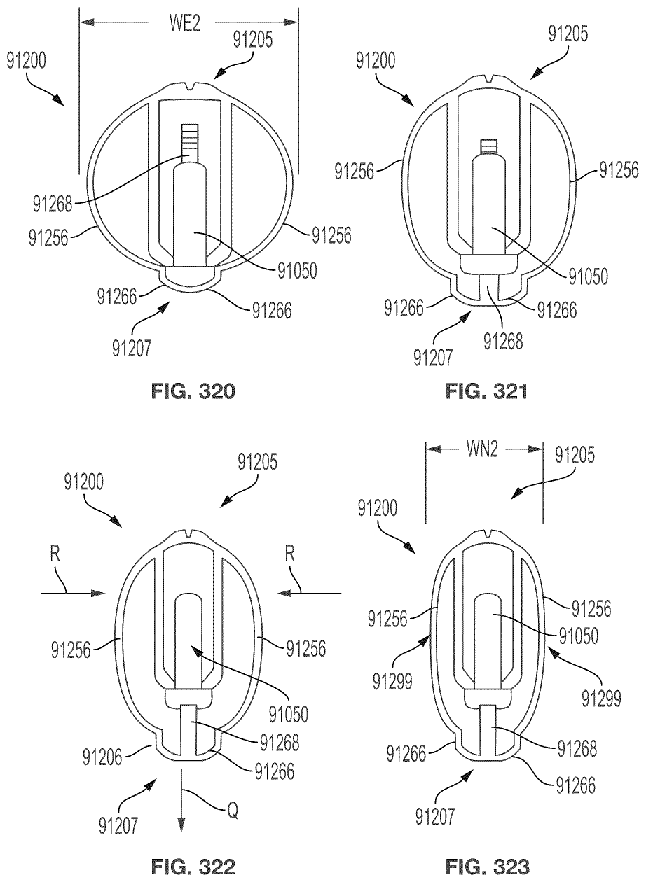

In the illustrated example, the paddle frames 91224 include outer frame members 91256 (e.g., curved struts, etc.) and inner frame members 91260 (e.g., struts, etc.). In FIG. 318, the outer frame members 91256 are shown in an expanded state such that the outer frame members 91256 define a paddle frame expanded width WE2 (FIG. 320).

The outer frame members 91256 are flexibly attached at the proximal portion 91205 and are flexibly attached at the distal portion 91207. The outer frame members 91256 are attached at the distal portion 91207 by connecting portions 91266. The outer frame members 91256 are curved and form a generally circular or oval shape. In some implementations, however, the outer frame members 91256 can be otherwise shaped.

The outer frame members 91256 also include an inner end or post 91268 extending axially along axis V from the distal portion 91207 toward the proximal portion 91205. The inner end or post 91268 can be configured in a variety of ways. In the illustrated example, the inner end or post 91268 has an outer surface 91269 that can be formed by a plurality of side walls forming a polygonal cross section or the outer surface 91269 can have one flat side and a-half cylindrical surface. The inner end or post 91268 can have an end surface 91271 that is perpendicular to the side walls.

The inner frame members 91260 extend from first connection portions 91270 with the outer frame members 91256 near or at the proximal portion 91205 and include second connection portions 91272 near or adjacent the distal portion 91207 that connect to the inner end or post 91268. The first frame side 91252 and a second frame side 91254 can be in contact with each other along axis T toward the distal end 91207 and are separated toward the proximal end 91205 to form a V-shape, as shown, for example, in FIG. 319.

The inner members 91209 can be a portion of a coaptation element, such as coaptation element 210 of FIGS. 22-27, or be attached to a coaptation element by any suitable means. As shown in FIG. 319, the outer paddles 91220 are jointably attached at the distal portion 91207 by connection portions 91221 and to the inner paddles 91222 by connection portions 91223. The inner paddles 91222 are flexibly attached to the inner members 91209 by connection portions 91225. The inner paddles 91222 and the inner members 91209 are not connected to the connection portions 91272, as shown in FIG. 319.

In this manner, the anchors are configured similar to legs in that the inner paddles 91222 are like upper portions of the legs, the outer paddles 91220 are like lower portions of the legs, and the connection portions 91223 are like knee portions of the legs

Referring to FIG. 318, the connection portions 91272 include a first retaining portion 91282 and a second retaining portion 91284, spaced apart from, and a mirror image of, the first retaining portion 91282. The inner frame members 91260 include inward transition portions 91290. In the illustrated example, the inward transition portions 91290 are formed as inward curved surfaces. In some implementations, however, the inward transition portions 91290 can be formed in any suitable manner, such as for example, as angled or tapered surfaces, stepped surfaces, or any other suitable inward transition.

The retaining portions 91282, 91284 extend axially from the inward transition portions 91290 toward the distal end 91207 to form a gap 91292 configured to receive the inner end or post 91268. Each of the retaining portions 91282, 91284 includes an outer recessed portion 91294. In the illustrated example, the recessed portions 91294 are formed axially nearer to the distal end 91207 than the inward transition portion 91290 is located.

The recessed portions 91294 are configured to receive an annular retainer 91098 of the inner end or post 91268 and the connecting portions 91221 of the outer paddles 91220. The annular retainer 91098 can be configured similar to the annular retainer 91098; thus, the description of the annular retainer 91098 applies equally to the annular retainer 91098. The annular retainer 91098 can be a ring, a washer, a nut, or that like that is connected to the inner end or post 91268. In the illustrated example, the annular retainer 91098 is integrally formed with the inner end or post 91268.

FIG. 318 illustrates an assembled state for the device 91200 in which the inner end or post 91268 is received through the gap 91292 in the connection portion 91272 and the retainer 91098 and the connecting portions 91221 of the outer paddles 91220 are received in the recessed portions 91294. The device 91200 is assembled in the same manner as the device 91000. For example, the inner end or post 91268 and connecting portion 91266 of the outer frame members 91256 and the connection portion 91272 of the inner frame members 91260 are pulled away from each other along the axis V.

The paddle frames 91224 can be made from a material that allows the inner end or post 91268 and connecting portion 91266 of the outer frame members 91256 and the connection portion 91272 of the inner frame members 91260 to be pulled away from each other. For example, the paddle frames 91224, or a portion thereof, can be made of a laser cut or otherwise formed flexible material, such as metal, plastic, etc.

In the illustrated example of FIGS. 318-323, the connecting portions 91266 are more rigid, such that the outer frame members 91256 and will retain their general shape more when the inner end or post 91268 is extended to narrow the outer frame members. The connecting portions 91266 can be configured in a variety of ways to be more rigid. For example, the thickness of the connecting portions 91266 and/or the material used in the connecting portions can provide more rigidity. In some implementations, the thickness of the connecting portion 91266 can be greater than the outer frame members 91256 to provide more rigidity. Further, in some implementations, the material used in the connecting portions 91266 can be a more rigid material to provide more rigidity.

Once the inner end or post 91268 and the connecting portion 91266 are separated from the connection portion 91272, the annular retainer 91098 and the connecting portions 91221 of the outer paddles 91220 can be placed therebetween and the distal ends of the first retaining portion 91282 and a second retaining portion 91284 can be compressed toward each other. In the compressed state, the annular retainer 91098 can be received over the first retaining portion 91282 and a second retaining portion 91284.

The annular retainer 91098 and the connecting portion 91221 of the outer paddles 91220 can be aligned with the recessed portions 91294 and the retaining portions 91282, 91284 can then be released to return toward the uncompressed state to capture the annular retainer 91098 and the connecting portion 91221 of the outer paddles 91220 in the recessed portions 91294.

Once the annular retainer 91098 and the connecting portions 91221 of the outer paddles 91220 are received in the recessed portions 91294, the inner end or post 91268 and the connecting portions 91266 of the outer frame members 91256 can be released, which allows the inner end or post 91268 to be received through the gap 91292 and the end surface 91271 extends past the inward transition portions 91290.

The width adjustment and actuation device 91050 of the device or implant 91200 is configured to both move the paddle frames 91224 between an expanded position and a narrowed position and to move the paddles between the closed position and the open position. The width adjustment and actuation device 91050 can be configured in a variety of ways. Any structure capable of selectively moving the paddle frames 91224 between an expanded position and a narrowed position and opening and closing the device can be used, such as for example, the width adjustment and actuation device 91050 of FIGS. 310-311. In some implementations, the width adjustment and actuation device are configured such that advancing and retracting the width adjustment and actuation device itself opens and closes the device and advancing and retracting a post inside the width adjustment and actuation device narrows and widens the paddles. For example, the inner paddle frame portions are moved by the width adjustment and actuation device to open and close the paddles in the same or a similar manner to that shown in FIGS. 23, 27, and 30-37.

Referring to FIG. 318, in the illustrated example the width adjustment and actuation device 91050 includes a receiver (e.g., an internally threaded element, a notched receiving portion, a column, a lumen, a tube, a shaft, a sleeve, a post, a housing, tracks, a cylinder, etc.), such as the illustrated sleeve 91202 configured to receive a portion of the inner end or post 91268 and a plug (not shown) configured to move the post axially within the sleeve 91202 to narrow and widen the paddles. The sleeve 91202 and the plug (not shown) can be configured the same or similar to the sleeve 91102 and the plug 91103 of the device 91000 of FIGS. 310-311, thus the description of the sleeve 91102 and the plug 91103 applies equally to the sleeve 91202 and the plug (not shown) of the example of FIGS. 318-323.

As shown in FIG. 318, the sleeve 91202 is fixedly attached to the connection portion 91272 such that the inner end or post 91268 can be received within a passage 91210 that extends through the sleeve 91202.

Referring to FIGS. 320-323, in operation, to move the device 91200 from the expanded position to the narrowed position, the plug (not shown) can be moved axially relative to the sleeve 91202. Movement of the plug toward the distal end 91207 causes the plug to engage the distal end 91271 of the inner end or post 91268 and move the inner end or post 91268 in the same direction (i.e., away from the proximal portion 91205 of the device).

Movement of the inner end or post 91268 away from the proximal portion 91205, as shown by arrow Q in FIG. 322, pulls the distal end portions of the outer frame members downward while the more rigid inner frame members maintain the positions of the proximal end portions of the outer frame members. As a result, the outer frame members 91256 are drawn inward (i.e., to the narrowed position), as shown by arrows R in FIG. 322. The device 91200 has a width WN2 (FIG. 323) in the narrowed position that is narrower than the width WE2 in the expanded position.

As shown in FIGS. 320-323, when the device 91200 moves between the expanded position and the narrowed position, the more rigid connecting portions 91266 tend to retain the shape, or deform only slightly, while the outer frame members 91256 move inward. As shown in FIG. 323, in the narrowed position, each of the outer frame members 91256 can optionally be configured to form a recessed or concave portion 91299 proximate the mid-point between the proximal portion 91205 and the distal portion 91207 when the outer frame members are contracted.

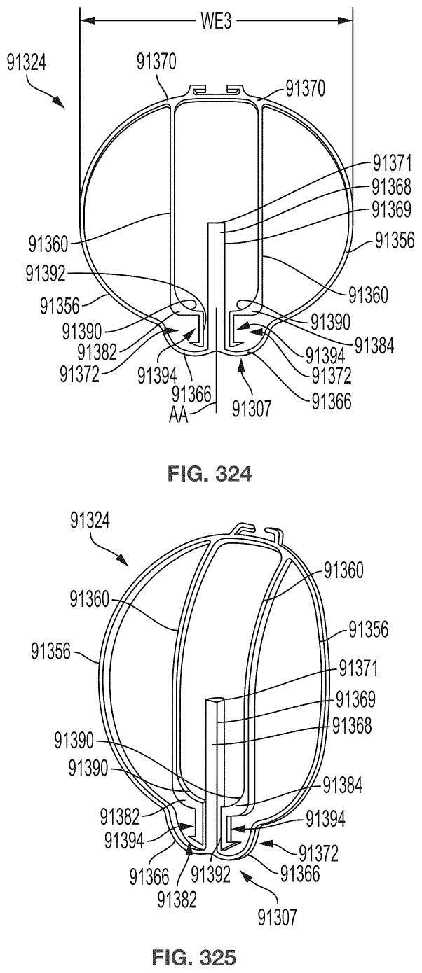

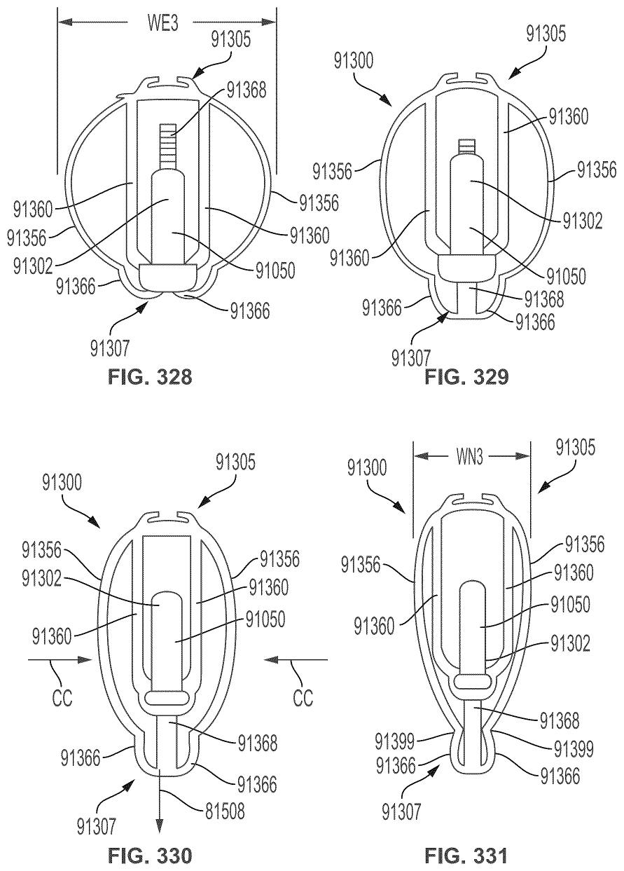

Referring to FIGS. 324-331, an example implementation of retractable/expandable paddle frames 91324 and a device or implant 91300 with the retractable/expandable paddle frames is shown. The device 91300 is similar to the device 91200 of FIGS. 318-323. Referring to FIGS. 328-331, the device or implant 91300 includes a proximal or attachment portion 91305, paddle frames 91324, a width adjustment and actuation device 91050, and a distal portion 91307. The width adjustment and actuation device 91050, and the paddle frames 91324 can be configured in a variety of ways. In some implementations, the width adjustment and actuation device are configured such that advancing and retracting the width adjustment and actuation device itself opens and closes the device and advancing and retracting a post inside the width adjustment and actuation device narrows and widens the paddles.

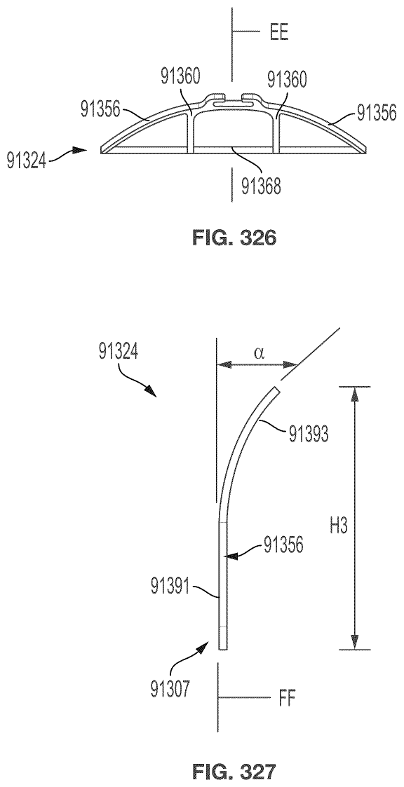

In the illustrated example of FIGS. 324-331, the paddle frames 91324 are symmetric along longitudinal axis AA (FIG. 324) and axis EE (FIG. 326). In some implementations of the device 91300, however, the paddle frames 91324 is not symmetric about the axes AA and EE.

In the illustrated example, the paddle frames 91324 include outer frame members 91356 (e.g., curved struts, etc.) and inner frame members 91360 (e.g., struts, etc.). In FIGS. 324 and 325, the outer frame members 91356 are shown in an expanded state such that the outer frame members 91356 define a paddle frame expanded width WE3 (FIG. 324). The outer frame members 91356 are flexibly attached to proximal ends of the inner frame members 91360 and are flexibly attached at the distal portion 91307. The outer frame members 91356 are attached at the distal portion 91307 by connecting portions 91366. The outer frame members 91356 are curved and form a generally circular or oval shape. In some implementations, however, the outer frame members 91356 can be otherwise shaped.

The outer frame members 91356 also include an inner end or post 91368 extending axially along axis AA from the distal portion 91307 toward the proximal portion 91305. The inner end or post 91368 can be configured in a variety of ways. In the illustrated example, the inner end or post 91368 includes an outer surface 91369 that can include a plurality of side walls forming a polygonal cross section or the outer surface 91369 can be semi-cylindrical with one flat surface. An end surface 91371 can be perpendicular to the outer surface 91369.

The inner frame members 91360 extend linearly from a connection portion 91370 with the outer frame members 91356 near or at the proximal portion 91305 and include retaining portions 91372 near or adjacent the distal portion 91307 for engaging the inner end or post 91368.

As shown in FIG. 327, the outer frame members 91356 and inner frame members 91360 at the proximal portion 91305 are angled or curved relative to the outer frame members 91356 and the inner frame members 91360 at the distal portion 91307 (see angle α). For example, the outer frame members 91356 and the inner frame members 91360 can extend linearly, or generally linearly, along a vertical axis FF from the distal portion 91307 toward the proximal portion 91305 defining a linear portion 91391 of the frame 91324.

Prior to a proximal end of the paddle frames 91324, the outer frame members 91356 and the inner frame members 91360 can begin to divert from the axis FF. In the illustrated example, the outer frame members 91356 and the inner frame members 91360 curve away from the axis FF along a curved portion 91393 of the frame 91324 such that the proximal portion 91305 of the outer frame members 91356 and the inner frame members 91360 are at an angle α relative to the axis FF. In the illustrated example, frame 91324 has a height H3 and the linear portion 91391 transitions to the curved portion 91393 in the range of 40-60% of the height H3. Further, in the illustrated example, the curvature of the curved portion 91393 of the outer frame members 91356 and the inner frame members 91360 is the same. In some implementations, however, the inner frame members 91360 can curve more or less than the outer frame members 91356.

The retaining portions 91372 include a first retaining portion 91382 and a second retaining portion 91384, spaced apart from, and a mirror image of, the first retaining portion 91382. Similar to the retaining portions 91282 of FIGS. 318-323, the inner frame members 91360 include inward transition portions 91390. In the illustrated example, the inward transition portions 91390 are formed as inward curved surfaces. In some implementations, however, the inward transition portions 91390 can be formed in any suitable manner, such as for example, as angled or tapered surfaces, stepped surfaces, or any other suitable inward transition.

The retaining portions 91382, 91384 extend distally from the inward transition portions 91390 to form a gap 91392 configured to receive the inner end or post 91368. Each of the retaining portions 91382, 91384 includes an outer recessed portion 91394. In the illustrated example, the recessed portions 91394 are formed axially nearer to the distal end 91307 than the inward transition portion 91390 is located.

The recessed portions 91394 are configured to receive an annular retainer (not shown) and outer paddles (not shown) in the same or similar manner as the recessed portions 91294 receive the annular retainer 91098 and the connecting portions 91221 of the outer paddles 91220 of the example of FIGS. 318-323.

The device 91300 is assembled in the same manner as the device 91200 of FIG. 318-323. For example, the inner end or post 91368 and connecting portion 91366 of the outer frame members 91356 and the retaining portion 91372 of the inner frame members 91360 are pulled away from each other along the axis AA.

The paddle frames 91324 can be made from a material that allows the inner end or post 91368 and connecting portion 91366 of the outer frame members 91356 and the retaining portion 91372 of the inner frame members 91360 to be pulled away from each other. For example, the paddle frames 91324, or a portion thereof, can be made from a flexible metal, plastic, etc. The material can be shape-memory alloy wire—such as Nitinol—to provide shape-setting capability, or any other flexible material suitable for implantation in the human body.

In the illustrated example of FIGS. 324-329, unlike the more rigid connecting portions 91266 of the example of FIGS. 318-323, the connecting portions 91366 are not configured to retain their shape during movement between the expanded position and the narrowed position. That is, the connecting portions 91366 are configured to flex substantially during movement between the expanded position and the narrowed position.

The width adjustment and actuation device 91050 of device or implant 91300 is configured to facilitate both moving the paddle frames 91324 between an expanded position and a narrowed position and moving the paddle frames of the device between the closed position and the open position. The width adjustment and actuation device 91050 can be configured in a variety of ways. Any structure capable of selectively moving the paddle frames 91324 between an expanded position and a narrowed position and cable of moving the device between the closed configuration and the open configuration can be used, such as for example, the width adjustment and actuation device 91050 of FIGS. 310-311. In some implementations, the width adjustment and actuation device are configured such that advancing and retracting the width adjustment and actuation device itself opens and closes the device and advancing and retracting a post inside the width adjustment and actuation device narrows and widens the paddles. For example, the inner paddle frame portions are moved by the width adjustment and actuation device to open and close the paddles in the same or a similar manner to that shown in FIGS. 23, 27, and 30-37.

Referring to FIGS. 328-331, in operation, to move the device 91300 from the expanded position to the narrowed position, the plug (not shown) is moved axially relative to a receiver (e.g., an internally threaded element, a notched receiving portion, a column, a lumen, a tube, a shaft, a sleeve, a post, a housing, tracks, a cylinder, etc.), such as the illustrated sleeve 91302 to engage the distal end 91371 of the inner end or post 91368 and move the inner end or post 91368 in the same direction (i.e., away from the proximal portion 91305 of the device).

Movement of the inner end or post 91368 away from the proximal portion 91305 pulls the distal end portions of the outer frame members downward while the inner frame members 91560 (e.g., struts, etc.) maintain the positions of the proximal end portions of the outer frame members (e.g., struts, etc.). As a result, the outer frame members 91356 are drawn inward (i.e., to the narrowed position), as shown by arrows CC in FIGS. 330, such that the device 91300 has a width WN3 (FIG. 331) in the narrowed position that is narrower than the width WE3 in the expanded position (FIG. 328).

As shown in FIGS. 328-331, when the device 91300 moves between the expanded position and the narrowed position, the connecting portions 91366 are pulled inward in a similar manner to the outer frame members 91356. As shown in FIG. 331, in the narrowed position, the connecting portions 91366 can form a recessed or concave portion 91399 proximate the distal portion 91307.

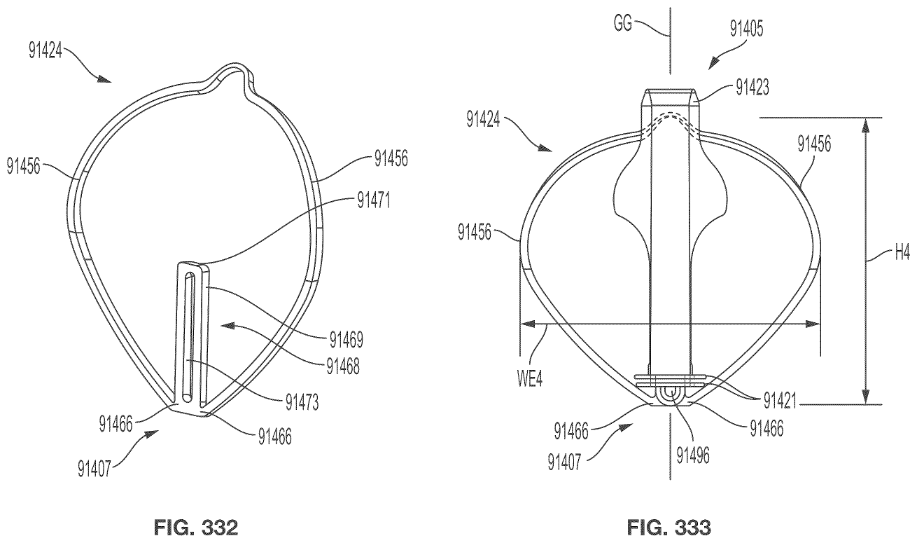

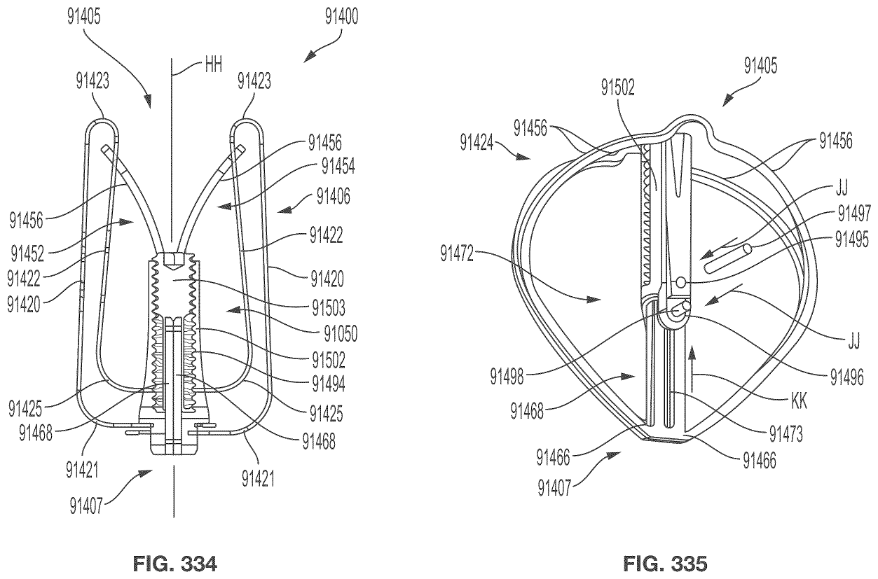

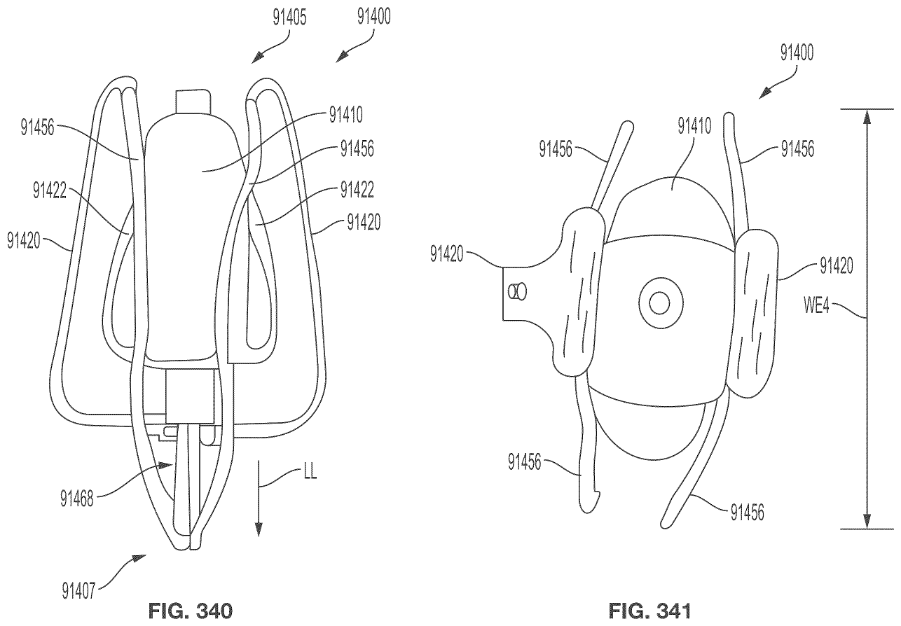

FIGS. 332-342 illustrate an example implementation of paddle frames 91424 and a device or implant 91400 that includes the paddle frames. Referring to FIG. 334, the device or implant 91400 includes a proximal or attachment portion 91405, an anchor portion 91406 (FIG. 334), the paddle frames 91424, a width adjustment and actuation device 91050, an optional coaptation element 91410 (FIG. 339) and a distal portion 91407. The paddle frames 91424 have a height H4 (FIG. 333) between the proximal portion 91405 and the distal portion 91407. Referring to FIG. 334, the anchor portion 91406 includes inner paddles 91422, and outer paddles 91420. The proximal portion 91405, the distal portion 91407, the anchor portion 91406, the width adjustment and actuation device 91050, and the paddle frames 91424 can be configured in a variety of ways.

In the illustrated example, the paddle frames 91424 are symmetric along longitudinal axis GG (FIG. 333) and are symmetric along longitudinal axis HH (FIG. 334). In some implementations of the device or implant 91400, however, the paddle frames 91424 are not symmetric about one or both of the axes GG and HH. Referring to FIG. 334, the paddle frames 91424 include a first frame side 91452 and a second frame side 91454 that is a mirror image of the first side 91452.

Referring to FIG. 332, the paddle frames 91424 include outer frame members 91456 (e.g., struts, etc.) with no inner rigid frame members. In FIG. 333, the outer frame members 91456 are shown in an expanded state such that the outer frame members 91256 define a paddle frame expanded width WE4 (FIG. 333). The outer frame members 91456 are flexibly attached at the proximal portion 91405 and are flexibly attached at the distal portion 91407. The outer frame members 91456 are attached at the distal portion 91407 by connecting portions 91466. The outer frame members 91456 are curved and form a generally oval, inverted teardrop, or circular shape. In some implementations, however, the outer frame members 91456 can be otherwise shaped.

The outer frame members 91456 also include an inner end or post 91468 extending axially along axis GG from the distal portion 91407 toward the proximal portion 91405. The inner end or post 91468 can be configured in a variety of ways. In the illustrated example, the inner end or post 91468 includes an outer surface 91469 that can include a plurality of side walls forming a polygonal cross section or the outer surface 91469 can be semi-cylindrical with one flat surface. An end surface 91471 can be perpendicular to the outer surface 91369. The illustrated inner end or post 91468 includes a longitudinally extending, closed-ended slot 91473.

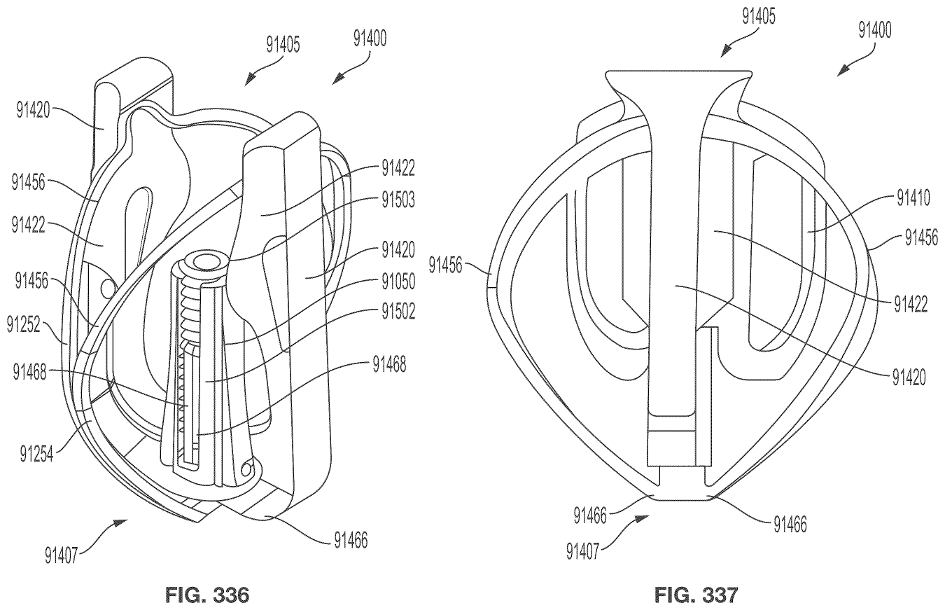

Referring to FIGS. 334 and 336, the first frame side 91252 and a second frame side 91254 are in contact with each other along axis HH toward the distal end 91407 and are separated toward the proximal end 91405 to form a V-shape.

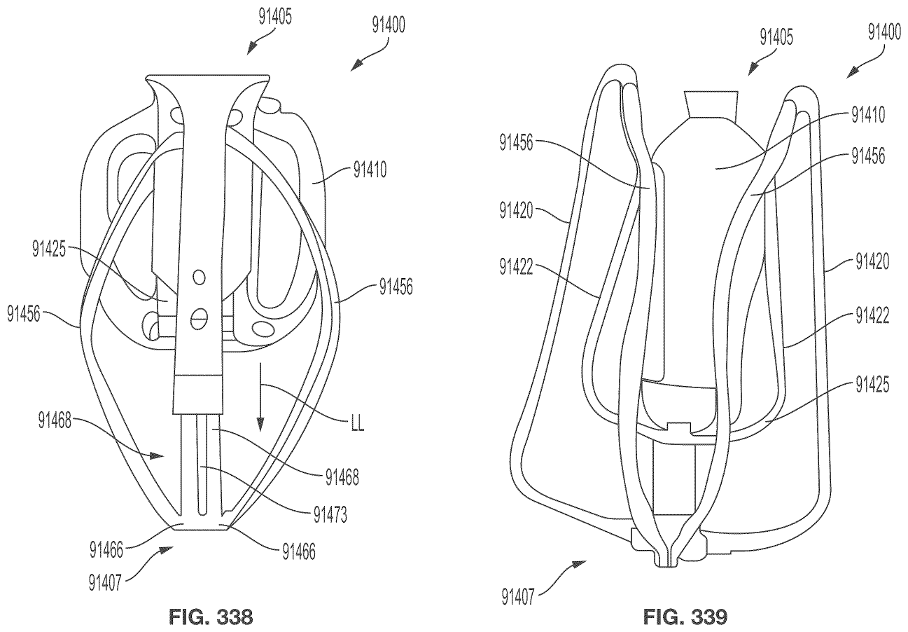

As shown in FIG. 334, the outer paddles 91420 are connected to the width adjustment and actuation device 91050 at the distal end 91407 via connection portions 91421 and to the inner paddles 91422 by connection portions 91423. The inner paddles 91422 are flexibly attached to a distal end of the coaptation element 91410 (See FIGS. 338 and 339) by connection portions 91425.

As shown in FIGS. 333 and 334, the connecting portions 91421 of the outer paddles 91420 is not jointly attached at the distal end 91407 but are offset similar to the connecting portions 91021 of FIGS. 301-302. In some implementations, however, the connecting portions 91421 of the outer paddles 91420 can be attached to one another at the distal end 91407.

In the illustrated example, the width adjustment and actuation device 91050 can be configured similar to the width adjustment and actuation device 91050 of FIGS. 301-310. For example, the width adjustment and actuation device 91050 can include a receiver (e.g., an internally threaded element, a notched receiving portion, a column, a lumen, a tube, a shaft, a sleeve, a post, a housing, tracks, a cylinder, etc.), such as the illustrated sleeve 91502 configured to receive a portion of the two posts 91468 and a coupler, such as the illustrated plug 91503 receivable within the sleeve 91502 and configured to move the posts 91468 axially within the sleeve 91502. The sleeve 91502 can be formed as part of the width adjustment and actuation device 91050.

The sleeve 91502 and the plug 91503 can be configured the same or similar to the sleeve 91102 and the plug 91103 of the device 91000 of FIGS. 310-311, thus the description of the sleeve 91102 and the plug 91103 applies equally to the sleeve 91502 and the plug 91503. As shown in FIGS. 335 and 336, the sleeve 91502 includes a passage 91494 that extends through the sleeve 91202 and is configured to receive the plug 91103.

Referring to FIG. 335, the device 91400 can include retaining portions 91472 configured to retain the inner end or post 91468 within the passage 91494. The retaining portions 91472 can be configured in a variety of ways. In the illustrated example, the retaining portions 91472 include a first pin bore 91495 and a second pin bore 91496 extending through the sleeve 91202 proximate the distal end 91407.

The first mounting pin 91497 is received through the first pin bore 91495, the second mounting pin 91498 is received through the second pin bore 91496, and both mounting pins 91497, 91498 are received through the slot 91473 in the inner end or post 91468, as shown by arrows JJ in FIG. 335.

Since the first and second pin bores 91495, 91496 are fixed relative to the sleeve 91502 and the first and the second mounting pins 91497, 91498 are received through the closed-ended slot 91473, the first and second mounting pins 91497, 91498 act as stops to prevent or inhibit the inner end or post 91468 from being fully retracted from the passage 91494 in the sleeve 91502 while allowing the inner end or post 91468 to move within the passage 91494, as shown by arrow KK in FIG. 335. Since there are two pins, the posts 91468 are prevented or inhibited from pivoting in the passage 91494. In addition, the connecting portions 91421 of the outer paddles 91420 are held between the first and second mounting pins 91497, 91498 to secure the outer paddles 91420 to the width adjustment and actuation device 91050. In an example implementation, only a single pin is included.

Referring to FIGS. 337-342, in operation, to move the device 91400 from the expanded position (as shown in FIGS. 337, 339, and 341) to the narrowed position (as shown in FIGS. 338, 340, and 342, the plug 91503 can be moved axially relative to the sleeve 91502 to engage the posts 91468 and move the posts in the same direction (i.e., away from the proximal portion 91405 of the device).

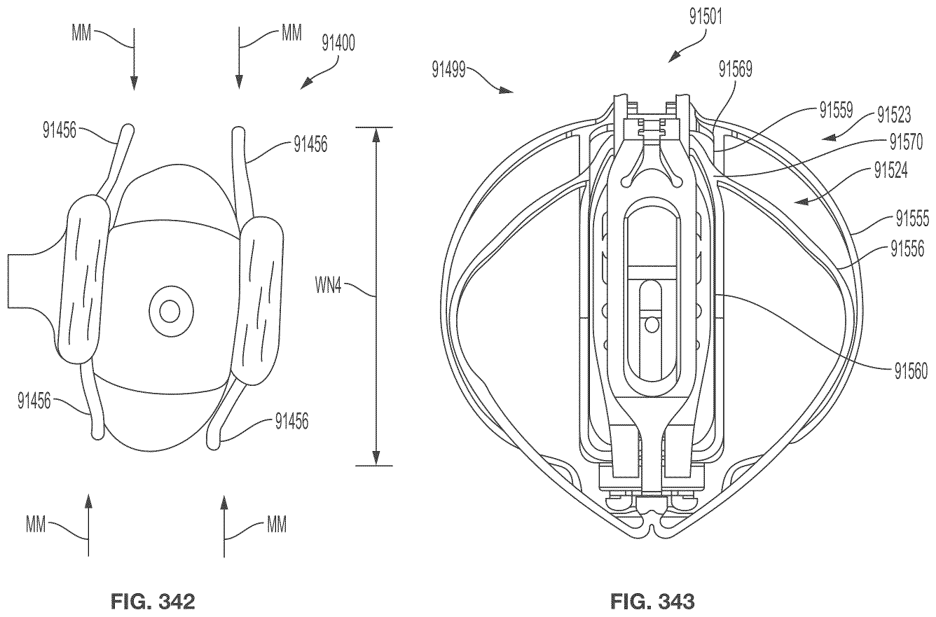

Movement of the inner end or post 91468 away from the proximal portion 91405, as shown by arrow LL in FIG. 338, pulls the distal end portions of the outer frame members downward. In this example, the inner paddle 91422, the outer paddle 91420, and/or another component that is attached to the inner or outer paddle portion(s) maintains or resists downward movement of the positions of the proximal end portions of the outer frame members. As a result, the outer frame members the outer frame members 91456 are drawn inward (i.e., to the narrowed position), as shown by arrows MM in FIG. 342, such that the device 91400 has a width WN4 (FIG. 342) in the narrowed position that is narrower than the width 9WE4 in the expanded position (FIG. 341).

Referring to FIG. 343, an illustration of a device or implant 91499 is shown illustrating that the device 91499 can utilize paddle frames of different configurations. In the illustrated example, the device 91499 is illustrated with a first paddle frame 91523 and with a second paddle frame 91524. The device 91499 would not utilize both paddle frame configurations at the same time. FIG. 343 is merely illustrative of the ability of devices or implants according to the present disclosure to utilize a variety of paddle frame configurations.

The first paddle frame 91523 is configured in a similar manner to the paddle frame 91224 of FIG. 324 in that the outer frame members 91555 (e.g., struts, etc.) has a circular or oval shape when in an expanded position and join inner frame members 91559 (e.g., struts, etc.) at a connection portion 91569 near or at the proximal portion 91501 of the frame 91523.

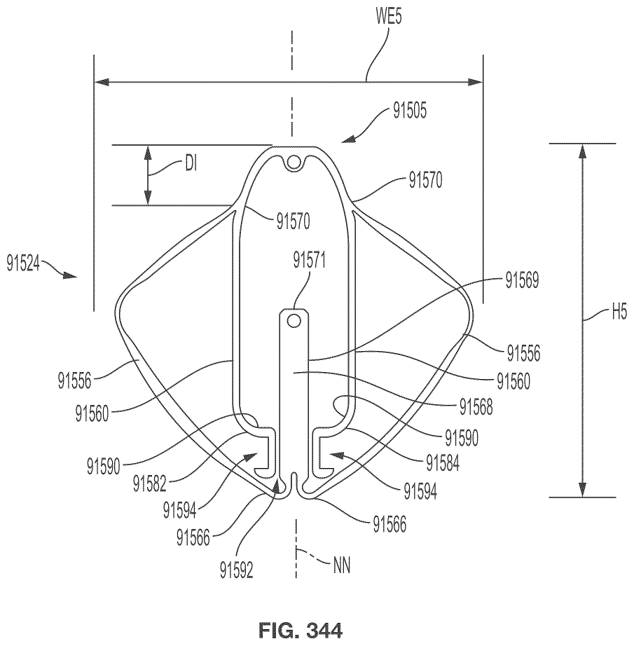

The second paddle frame 91524, which is illustrated in more detail in FIG. 344, has outer frame members 91556 that form a diamond, rhombus, or kite-like shape when in an expanded position and join inner frame members 91560 at a connection portion 91570 along the inner frame member 91560 spaced away from the proximal portion 91501 a distance D1 (FIG. 344).

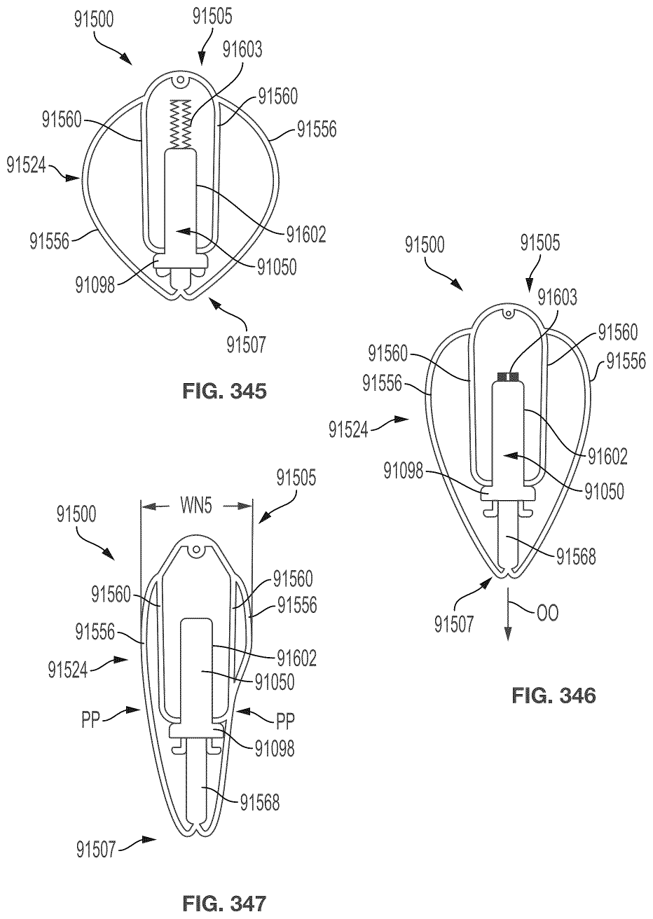

Referring to FIGS. 344-347, an example implementation of paddle frames 91524 and a device or implant 91500 utilizing the paddle frames 91524 is shown. Referring to FIGS. 345-347, the device or implant 91500 includes a proximal or attachment portion 91505, the paddle frames 91524, a width adjustment and actuation device 91050, and a distal portion 91507. The paddle frames 91524 have a height H5 (FIG. 344) between the proximal portion 91505 and the distal portion 91507. The device 91500 can include an anchor portion (not shown). The anchor portion (not shown) can include any of the features of the anchor portions described in this application. The proximal portion 91505, the distal portion 91507, the width adjustment and actuation device 91050, and the paddle frames 91524 can be configured in a variety of ways.

In the illustrated example of FIGS. 344-347, the paddle frames 91524 are symmetric along longitudinal axis NN (FIG. 344). In some implementations of the device or implant 91500, however, the paddle frames 91524 is not symmetric about the axis NN.

In FIG. 344, the outer frame members 91556 are shown in an expanded state such that the outer frame members 91556 define a paddle frame expanded width WE5 (FIG. 344). The outer frame members 91556 are flexibly attached at a proximal portion of the inner frame members and are flexibly attached at distal ends by connecting portions 91566.

The outer frame members 91556 also include an inner end or post 91568 extending axially along axis NN from the distal portion 91507 toward the proximal portion 91505. The inner end or post 91568 can be configured in a variety of ways. In some implementations, each post can include a flat surface faces the flat surface of the post of the second paddle frame.

The inner frame members 91560 extend from the connection portion 91570 with the outer frame members 91556 that is located at the distance D1 below the proximal portion 91505 along the inner frame members 91560.

Referring to FIG. 344, the inner frame members 91560 include a first retaining portion 91582 and a second retaining portion 91584, spaced apart from, and a mirror image of, the first retaining portion 91582. Similar to the retaining portions 91082 of FIGS. 29-34, the inner frame members 91560 include inward transition portions 91590. In the illustrated example, the inward transition portions 91590 are formed as inward curved surfaces. In some implementations, however, the inward transition portions 91590 can be formed in any suitable manner, such as for example, as angled or tapered surfaces, stepped surfaces, or any other suitable inward transition.

The retaining portions 91582, 91584 extend axially from the inward transition portions 91590 toward the distal end 91507 to form a gap 91592 configured to receive the inner end or post 91568.

Each of the retaining portions 91582, 91584 include an outer recessed portion 91594. In the illustrated example, the recessed portions 91594 are formed axially nearer to the distal end 91507 than the inward transition portion 91590 is located.

The recessed portions 91594 are configured to receive an annular retainer (not shown), such as for example, the annular retainer 91098 of the device 91000, and/or engage the width adjustment and actuation device 91050 to attach the inner frame members 91560 to the width adjustment and actuation device 91050. FIG. 345 illustrates an assembled state for the device 91500. In the assembled state, the inner end or post 91568 is received through the gap 91592 (see FIG. 345). The device 91500 can be assembled in the same manner as described regarding the device 91000.

The paddle frames 91524 can be made from a material that allows the inner end or post 91568 and connecting portion 91566 of the outer frame members 91556 and the retaining portion 91582 of the inner frame members 91560 to be pulled away from each other. For example, the paddle frames 91524, or a portion thereof, can be formed using any suitable processes, such as cutting, molding, casting, shape-setting, etc.

The width adjustment and actuation device 91050 of the device or implant 91500 is configured to facilitate both moving the paddle frames 91524 between an expanded position and a narrowed position and to move the paddles of the device between the closed position and the open position. The width adjustment and actuation device 91050 can be configured in a variety of ways. Any structure capable of selectively moving the paddle frames 91524 between an expanded position and a narrowed position and also opening and closing the device paddles can be used. For example, the inner paddle frame portions are moved by the width adjustment and actuation device to open and close the paddles in the same or a similar manner to that shown in FIGS. 23, 27, and 30-37. The width adjustment and actuation device 91050 can utilize any of the features of any width adjustment and actuation device described in this application, such as a coupler (e.g., the illustrated plug 91603) in a receiver (e.g., an internally threaded element, a notched receiving portion, a column, a lumen, a tube, a shaft, a sleeve, a post, a housing, tracks, a cylinder, etc.), such as the illustrated sleeve 91602 (FIG. 345).

Referring to FIGS. 345-347, in operation, to move the outer paddles of the device 91500 from the expanded position to the narrowed position, the plug 91603 is moved axially relative to the sleeve 91602. Movement of the plug 91603 toward the distal end 91507 causes the plug 91603 to engage the end 91571 of the inner end or post 91568 and move the inner end or post 91568 in the same direction (i.e., away from the proximal portion 91505 of the device).

Movement of the inner end or post 91568 away from the proximal portion 91505, as shown by arrow OO in FIG. 346, pulls the distal end portions of the outer frame members 91556 downward while the inner frame members 91560 maintain the positions of the proximal end portions of the outer frame members. As a result, the outer frame members 91556 are drawn inward (i.e., to the narrowed position), as shown by arrows PP in FIG. 347, such that the device 91500 has a width WN5 (FIG. 347) in the narrowed position that is narrower than the width WE5 in the expanded position.

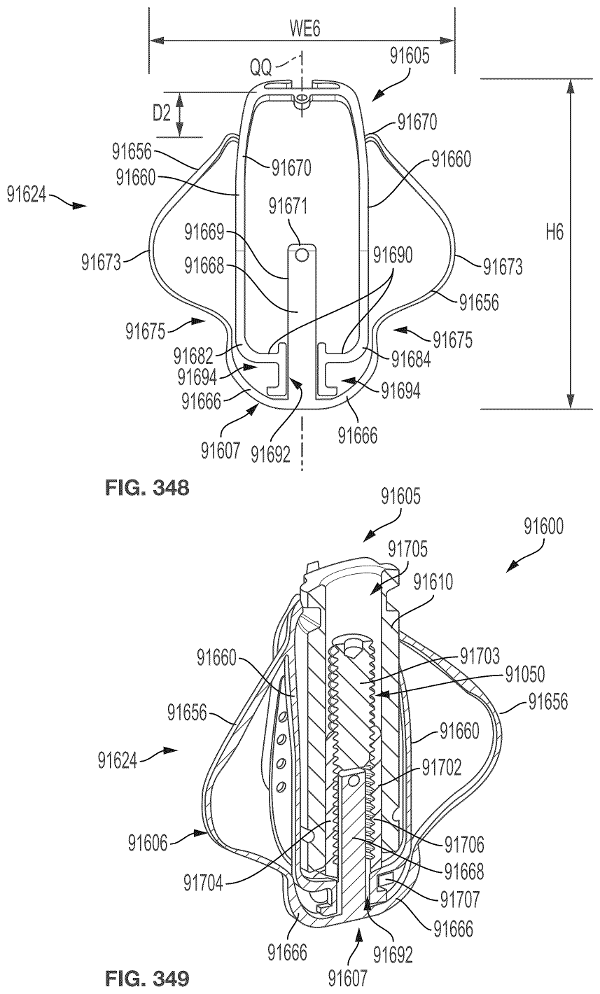

Referring to FIGS. 348-349, an example implementation of paddle frames 91624 and a device or implant 91600 that uses the paddle frames is shown. Referring to FIG. 349, the device or implant 91600 includes a proximal or attachment portion 91605, paddle frames 91624, a width adjustment and actuation device 91050, a central post, spacer, coaption element, coaptation element, etc. 91610, and a distal portion 91607. The paddle frames 91624 have a height H6 (FIG. 348) between the proximal portion 91605 and the distal portion 91607. The device 91600 can include an anchor portion 91606. The anchor portion 91606 can include any of the features of the anchor portions described in this application. The proximal portion 91605, the distal portion 91607, the width adjustment and actuation device 91050, and the paddle frames 91624 can be configured in a variety of ways.

In the illustrated example, the paddle frames 91624 are symmetric along longitudinal axis QQ (FIG. 344). In some implementations of the device or implant 91600, however, the paddle frames 91624 is not symmetric about the axis QQ.

In the illustrated example, the paddle frames 91624 include outer frame members 91656 (e.g., struts, etc.) and inner frame members 91660 (e.g., struts, etc.). In FIG. 348, the outer frame members 91656 are shown in an expanded state such that the outer frame members 91656 define a paddle frame expanded width WE6.

The outer frame members 91656 are flexibly attached at the distal portion 91607 by connecting portions 91666 and are joined to the inner frame members 91660 by a connection portion 91670 a distance D2 from the proximal portion 91605 in the direction of the distal portion 91607. From the connection portion 91670, the outer frame members 91656 form a curved, convex portion 91673 that transitions to a curved concave portion 91675 prior to transitioning to the connection portion 91666.

The outer frame members 91656 also include an inner end or post 91668 extending axially along axis QQ from the distal portion 91607 toward the proximal portion 91605. The inner end or post 91668 can be configured in a variety of ways. can be configured in a variety of ways. In some implementations, each post can include a flat surface that faces the flat surface of the post of the second paddle frame. In the illustrated example, the inner end or post 91668 includes an outer elongated surface 91669 and an end surface 91671 perpendicular to the outer elongated surface.

The inner frame members 91660 extend from the proximal portion 91605 toward the distal portion 91607. The inner frame members 91660 include a first retaining portion 91682 and a second retaining portion 91684, spaced apart from, and a mirror image of, the first retaining portion 91682. The inner frame members 91660 include inward transition portions 91690. In the illustrated example, the inward transition portions 91690 are formed as inward curved surfaces. In some implementations, however, the inward transition portions 91690 can be formed in any suitable manner, such as for example, as angled or tapered surfaces, stepped surfaces, or any other suitable inward transition.

The retaining portions 91682, 91684 extend axially from the inward transition portions 91690 toward the distal end 91607 to form a gap 91692 configured to receive the inner end or post 91668.

Each of the retaining portions 91682, 91684 includes an outer recessed portion 91694. In the illustrated example, the recessed portions 91694 are formed axially nearer to the distal end 91607 than the inward transition portion 91690 is located. The recessed portions 91694 are configured to engage and attach the inner frame members to a portion of the width adjustment and actuation device 91050, as described below.

The paddle frames 91624 can be made from a flexible material that allows the inner end or post 91668 and connecting portion 91666 of the outer frame members 91656 moved away from proximal portion 91605. For example, the paddle frames 91624, or a portion thereof, can be made of a metal, plastic, etc. and can formed in any suitable way, such as by cutting, molding, casting, and/or shape setting, etc.

The width adjustment and actuation device 91050 of the device or implant 91600 is configured to facilitate both moving the paddle frames 91624 between an expanded position and a narrowed position and to move the paddles of the device 91600 between a closed position and an open position. The width adjustment and actuation device 91050 can be configured in a variety of ways. Any structure capable of selectively moving the paddle frames 91624 between an expanded position and a narrowed position and moving the paddles between the open and closed positions can be used. In the example illustrated by FIG. 349, the width adjustment and actuation device 91050 pushes the inner end or post 91668 out of the receiver, such as the illustrated sleeve 91702, to narrow the paddles and the sleeve is slidable/pushable out of the central post, spacer, coaption element, coaptation element, etc. 91610 to open the paddles of the device. For example, the inner paddle frame portions are moved by the width adjustment and actuation device to open and close the paddles in the same or a similar manner to that shown in FIGS. 23, 27, and 30-37. The width adjustment and actuation device 91050 can utilize any of the features of any width adjustment and actuation device described in this application.

In the illustrated example, the width adjustment and actuation device 91050 includes a sleeve 91702 configured to receive a portion of the inner end or post 91668 and a coupler, such as the illustrated plug 91703, configured to move the post axially within the sleeve 91702. The sleeve 91702 can be configured in a variety of ways. In the illustrated example, the sleeve 91702 includes a cylindrical sidewall 91704 configured to be received within a passage 91705 in the post, spacer, coaption element, coaptation element, etc. 91610. The sidewall 91704 forms an internal threaded passage 91706 that extends through the entire length of the sleeve 91702. The internal threaded passage 91706 is configured to receive the inner end or post 91668 and the plug 91703.

The sleeve can 91702 include an attachment portion 91707 configured to engage and be received in the outer recessed portion 91694 in a similar manner as the annular retainer 91098 is received in the recessed portion 91094 of the device 91000. As a result, the sleeve 91702 is fixed to the inner frame portion 91660.

As shown in FIG. 349, the plug 91703 is received within the passage 91706. The plug 91703 is configured to move axially (e.g., due to rotating the threaded plug) within the sleeve 91702 to engage the distal end 91671 of the inner end or post 91668. The inner end or post 91668 is received in the passage 91706 of the sleeve 91702. Movement of the device 91600 from the expanded position to the narrowed position, can be accomplished by moving the plug 91703 axially relative to the sleeve 91602 toward the distal end 91607 to move the inner end or post 91668 in the same direction (i.e., away from the proximal portion 91605 of the device). Movement of the inner end or post 91668 away from the proximal portion 91605 pulls the distal ends of the outer frame members 91656 while the positions of the proximal ends of outer frame members are maintained by their connection to the inner frame members 91660. As a result, the outer frame members 91656 are drawn inward (i.e., to the narrowed position). To open and close the paddles of the device relative to the central post, spacer, coaption element, coaptation element, etc. 91610, the entire width adjustment device is advanced (to open) out of or away from the central post, spacer, coaption element, coaptation element, etc. 91610 or retracted (to close) toward or into the central post, spacer, coaption element, coaptation element, etc. 91610.

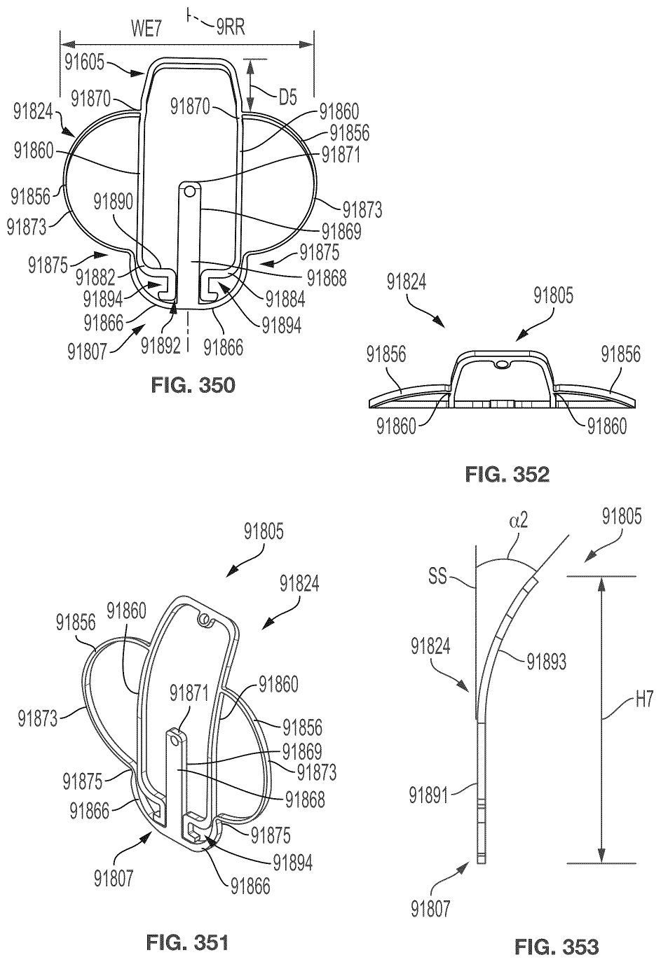

Referring to FIGS. 350-353, an example implementation of a paddle frame 91824 for a device or implant is shown. The paddle frame 91824 can be configured in a variety of ways and can be used with any of the devices or implants described in this application.

In the illustrated example of FIGS. 350-353, the paddle frames 91824 are symmetric along longitudinal axis RR (FIG. 350). In some implementations, however, the paddle frames 91824 is not symmetric about the axis RR.

In the illustrated example, the paddle frame 91824 includes outer frame members 91856 (e.g., struts, etc.) and inner frame members 91860 (e.g., struts, etc.). In FIG. 350, the outer frame members 91856 are shown in an expanded state such that the outer frame members 91856 define a paddle frame expanded width WE7. The outer frame members 91856 can expand and contract in the same or a similar manner to any of the other examples described herein and can be used in any of the devices described herein.

The outer frame members 91856 are flexibly attached at the distal portion 91807 by connecting portions 91866 and are joined to the inner frame members 91860 by a connection portion 91870 a distance D5 from the proximal portion 91805 in the direction of the distal portion 91807. From the connection portion 91870, the outer frame members 91856 form a curved, convex portion 91873, such as a semi-circular portion, that transitions to a curved, concave portion 91875 prior to transitioning to the connection portion 91866.

The outer frame members 91856 also include an inner end or projection 91868 extending axially along axis RR from the distal portion 91807 toward the proximal portion 91805. The inner end or projection 91868 can be configured in a variety of ways. In the illustrated example, the inner end or projection 91868 includes a plurality of side walls 91869 forming a rectangular cross section and an end surface 91871 perpendicular to the side walls.

The inner frame members 91860 extend from the proximal portion 91805 toward the distal portion 91807. The inner frame members 91860 include a first retaining portion 91882 and a second retaining portion 91884, spaced apart from, and a mirror image of, the first retaining portion 91882. The inner frame members 91860 include inward transition portions 91890. In the illustrated example, the inward transition portions 91890 are formed as inward curved surfaces. In some implementations, however, the inward transition portions 91890 can be formed in any suitable manner, such as for example, as angled or tapered surfaces, stepped surfaces, or any other suitable inward transition.

The retaining portions 91882, 91884 extend axially from the inward transition portions 91890 toward the distal end 91807 to form a gap 91892 configured to receive the inner end or projection 91868. Each of the retaining portions 91882, 91884 includes an outer recessed portion 91894. In the illustrated example, the recessed portions 91894 are formed axially nearer to the distal end 91807 than the inward transition portion 91890 is located. The recessed portions 91894 are configured to engage and attach the inner frame members to a portion of the width adjustment and actuation device in a similar manner as described herein with other recessed portions of the paddle frames.

The paddle frames 91824 can be made from a material that allows the inner end or projection 91868 and connecting portion 91866 of the outer frame members 91856 to be moved away from proximal portion 91805. For example, the paddle frames 91824, or a portion thereof, can be made of a metal, plastic, etc. The paddle frames can be formed in any suitable way, such as by cutting, such as laser cutting, molding, casting, shape setting, etc.

As shown in FIGS. 351-353, the portions of the outer frame members 91856 and the inner frame members 91860 at the proximal portion 91805 are angled or curved relative to the portions of the outer frame members 91856 and the inner frame members 91860 at the distal portion 91807. For example, the outer frame members 91856 and the inner frame members 91860 can extend linearly, or generally linearly, along a vertical axis SS from the distal portion 91807 toward the proximal portion 91805 defining a linear portion 91891 of the frame 91824 (FIG. 353). Prior to reaching the proximal portion 91805, the outer frame members 91856 and the inner frame members 91860 can begin to diverge from the axis SS. In the illustrated example, the outer frame members 91856 and the inner frame members 91860 curve away from the axis SS along a curved portion 91893 of the frame 91324 such that the proximal portion 91305 of the outer frame members 91856 and the inner frame members 91860 are at an angle α2 relative to the axis SS.

In the illustrated example, frame 91824 has a height H7 and the linear portion 91891 transitions to the curved portion 91893 in the range of 40-60% of the height H7. Further, in the illustrated example, the curvature of the curved portion 91893 of the outer frame members 91856 and the inner frame members 91860 is the same. In some implementations, however, the inner frame members 91860 can curve more or less than the outer frame members 91856.

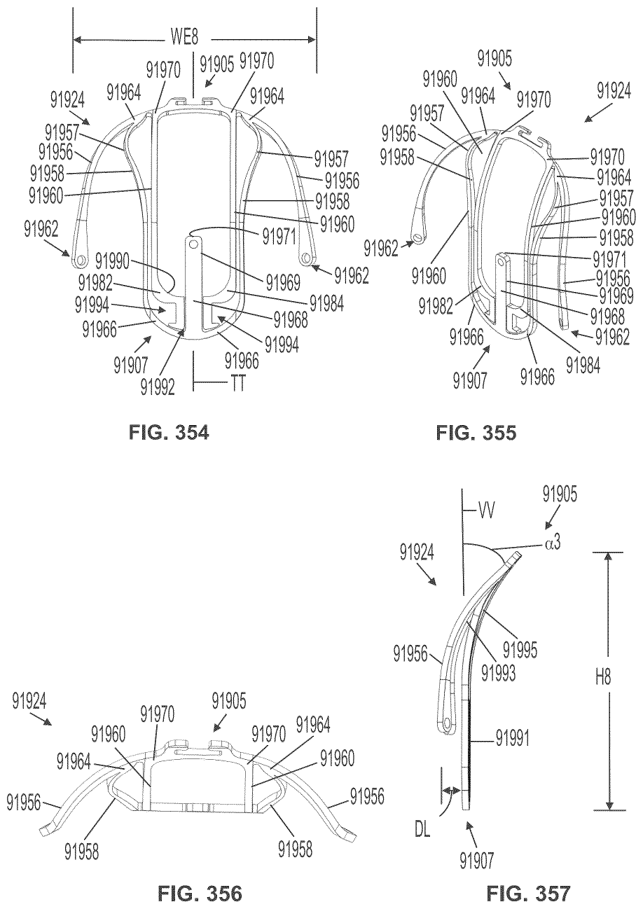

Referring to FIGS. 354-357, an example implementation of a paddle frame 91924 for a device or implant is shown. The paddle frame 91924 can be configured in a variety of ways and can be used with any of the devices or implants described in this application.

In the illustrated example of FIGS. 354-357, the paddle frames 91924 are symmetric along longitudinal axis TT (FIG. 354). In some implementations, however, the paddle frames 91924 is not symmetric about the axis TT.

In the illustrated example, the paddle frames 91924 includes outer frame members 91956 (e.g., arms, etc.), intermediate frame members 91958 (e.g., struts, etc.), and inner frame members 91960 (e.g., struts, etc.). In FIG. 354, the outer frame members 91956 are shown in an expanded state such that the outer frame members 91956 define a paddle frame width WE8 in the expanded state. The outer frame members 91856 can expand and contract in the same or a similar manner to any of the other examples described herein and can be used in any of the devices described herein.

The outer frame members 91956 are attached to the intermediate frame members 91958 at the proximal portion 91905 and include a free terminal distal end 91962. The outer frame members 91956 are curved and form a semicircle or U-shape. In some implementations, however, the outer frame members 91956 can be otherwise shaped.

The intermediate frame members 91958 extend from a connection portion 91964 with the outer frame members 91956 near or at the proximal portion 91905 and are attached at the distal portion 91907 via connection portions 91966. In the illustrated example, the intermediate frame members 91958 include a convex curved portion 91957 proximal to the connection portion 91964.

The intermediate frame members 91958 also include a projection or post 91968 extending axially along axis TT from the distal portion 91907 toward the proximal portion 91905. The post 91968 can be configured in a variety of ways. In the illustrated example, the post 91968 has a plurality of side walls 91969 and an end surface 91971 perpendicular to the outer side surface.

The inner frame members 91960 extend from a connection portion 91970 with the outer frame members 91956 near or at the proximal portion 91905 and include a first retaining portion 91982 and a second retaining portion 91984, spaced apart from, and a mirror image of, the first retaining portion 91982. Similar to the retaining portions of FIGS. 29-34, the inner frame members 91960 include inward transition portions 91990 that form a seat. In the illustrated example, the inward transition portions 91990 are formed as inward curved surfaces. In some implementations, however, the inward transition portions 91990 can be formed in any suitable manner, such as for example, as angled or tapered surfaces, stepped surfaces, or any other suitable inward transition.

The retaining portions 91982, 91984 extend axially from the inward transition portions 91990 toward the distal end 91907 to form a gap 91992 configured to receive the post 91968.

Each of the retaining portions 91982, 91984 includes an outer recessed portion 91994. In the illustrated example, the recessed portions 91994 are formed axially nearer to the distal end 91907 than the inward transition portion 91990 is located.

The paddle frames 91924 can be made from a material that allows the post 91968 and connecting portion 91966 of the outer frame members 91956 to be moved away from one another to accept an actuating portion, such as any of the actuating portions described above. For example, the paddle frames 91924, or a portion thereof, can be made of a flexible metal, plastic, etc. The paddle frames 91924 can be cut from a flat sheet of material, such as by laser cutting. The paddles can be made from a shape-memory material—such as Nitinol—and shape set to any desired shape.

As shown in FIG. 355-357, proximal portions of the outer frame members 91956, the intermediate frame members 91958, and the inner frame members 91960, are angled relative to distal portions of the outer frame members 91956, the intermediate frame members 91958, and the inner frame members 91960. For example, the outer frame members 91956, the intermediate frame members 91958, and the inner frame members 91960 can extend linearly, or generally linearly, along, or parallel to a vertical axis VV from the distal portion 91907 toward the proximal portion 91905 defining a linear portion 91991 of the frame 91824 (FIG. 357). As shown in FIG. 357, in the illustrated example, the outer frame members 91956 can be laterally offset from the intermediate frame members 91958 and the inner frame members 91960 a distance DL.

Prior to reaching the proximal portion 91905, the outer frame members 91956, the intermediate frame members 91958, and the inner frame members 91960 can begin to diverge from extending parallel to the axis VV. In the illustrated example, the outer frame members 91956, the intermediate frame members 91958, and the inner frame members 91960 curve away from being parallel to the axis VV along a curved portion 91993 for the outer frame member 91956 and a curved portion 91995 for the intermediate frame members 91958 and the inner frame members 91960. As shown in FIG. 357, at the proximal portion 91905 the outer frame members 91956, the intermediate frame members 91958, and the inner frame members 91960 are at an angle α3 relative to the axis VV. In the illustrated example, frame 91924 has a height H8 and the linear portion 91991 transitions to the curved portions 91993, 91995 in the range of 40-80% of the height H8. Further, due to the offset of the outer frame members 91956, the curvature of the curved portion 91993 is greater than the curvature of the curved portion 91995.

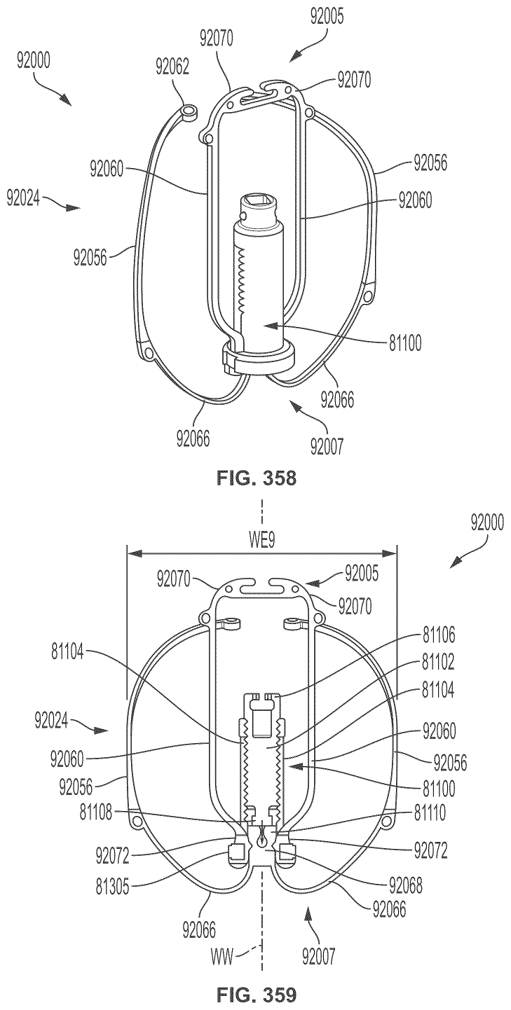

Referring to FIGS. 358-359, an example implementation of a device or implant 92000 is shown. The device or implant 92000 includes a proximal or attachment portion 92005, paddle frames 92024, a width adjustment and actuation device 81100, and a distal portion 92007. The attachment portion 92005, the distal portion 92007, the width adjustment and actuation device 81100, and the paddle frames 92024 can be configured in a variety of ways.

In the illustrated example of FIGS. 358-359, the paddle frames 92024 are symmetric along longitudinal axis WW (FIG. 359). In some implementations of the device or implant 92000, however, the paddle frames 92024 is not symmetric about the axis WW.

In the illustrated example, the paddle frames 92024 include outer frame members 92056 (e.g., arms, etc.) and inner frame members 92060 (e.g., struts, etc.). In FIGS. 358-359, the outer frame members 92056 are shown in an expanded state such that the outer frame members 92056 define a paddle frame expanded width WE9 (FIG. 359).

The outer frame members 92056 are flexibly attached at the distal portion 92007 via connection portions 92066. The outer frame members 92056 extend from the connection portions 92066 to terminal distal end 92062 that is at or adjacent the proximal portion 92005. These ends 92062 can optionally be connected to the inner frame members. For example, the ends 92062 can be connected to the inner frame members 92060 by hinge connections, by lines or sutures, by a covering that covers the outer frame members 92056 and the inner frame members 92060, etc.

Between the connection portions 92066 and the terminal distal ends 92062, the outer frame members 92056 form a curved, convex shape (e.g., circle or oval). In some implementations, however, the outer frame members 92056 can be otherwise shaped. As shown in FIG. 359, the terminal distal ends 92062 are positioned behind the inner frame members 92060.

The outer frame members 92056 also include a moveable member, which in the illustrated example, acts as an inner end or attachment portion 92068 configured to attach to the width adjustment and actuation device 81100. The inner end or attachment portion 92068 can be configured in a variety of ways. Any configuration that can suitably attach the outer frame members 92056 to the width adjustment and actuation device 81100 to allow the width adjustment and actuation device 81100 to move the outer frame members 92056 between a narrowed position and an expanded position can be used.

The width adjustment and actuation device 81100 that is configured to expand or contract the outer paddle frames 92056 of the device or implant 92000. In the illustrated example, the width adjustment and actuation device 81100 includes a coupler, such as the illustrated externally threaded shaft 81102 that is rotatably engaged with a receiver (e.g., an internally threaded element, a notched receiving portion, a column, a lumen, a tube, a shaft, a sleeve, a post, a housing, tracks, a cylinder, etc.), such as the illustrated internally threaded element 81104. The threaded element 81104 can be integrally formed with the distal cap, or retainer 81305.

A driver head 81106 is disposed at a proximal end of the shaft 81102 and is configured to rotatably drive the shaft 81102 into or out of the threaded element or column 81104. A coupler 81108 is attached to a distal end of the shaft 81102 and is configured to be retained by a receiver 81110 that is connected to the outer paddle frames 92056. In this way, when the driver head 81106 is driven to rotate the shaft 81102 counterclockwise (e.g., right-handed thread configuration), the shaft 81102 will rotate and move toward a proximal end of the width adjustment and actuation device 81100 causing the coupler 81108 to pull on the receiver 81110 and the distal ends of the outer paddle frames 92056. As the receiver 81110 is pulled by the coupler 81108, the outer paddle frames 92056 will pull into (or move in a first direction relative to) threaded element or column 81104. Conversely, rotating the shaft 81102 clockwise (e.g., distally into the threaded element or column 81104) will push the outer paddle portions out of (or in a second direction relative to) the threaded element or column 81104. However, it should be understood that other configurations are also contemplated.

The inner frame members 92060 are attached at the proximal portion 92005 via connection portions 92070 and extend from the connection portions 92070 to the distal portion 92007. The inner frame members 92060 include retaining portions 92072 near or adjacent the distal portion 92007 for attaching to the width adjustment and actuation device 81100. The retaining portions 92072 and the width adjustment and actuation device 81100 can be configured to attach in any suitable manner, such as for example, similar to how the retaining portions 91682, 91684 attach to the attachment portion 91707 of the sleeve 91702 of the device 91600.

The width adjustment and actuation device 81100 is configured to move the outer frame members 92056 from the expanded position to the narrowed position by pulling the inner end or attachment portion 92068 and portions of the connecting portions 92066 into the width adjustment and actuation device 81100. The inner paddle frame portions 92060 are moved by the width adjustment and actuation device to open and close the paddles in the same or a similar manner to that shown in FIGS. 23, 27, and 30-37.

The paddle frames 92024 can be made from a material that allows the inner end or attachment portion 92068 and portions of the connecting portions 92066 to be pulled into the width adjustment and actuation device 81100. For example, the paddle frames 92024, or a portion thereof, can be made of any flexible material, including but not limited to, metal, plastic, fabric, suture, etc. The paddle frames can be made using a variety of processes, including, but not limited to, cutting, such as laser cutting, stamping, casting, molding, heat treating, shape setting, etc. The paddle frames can be made from a shape memory material, such as—such as Nitinol—to provide shape-setting capability.

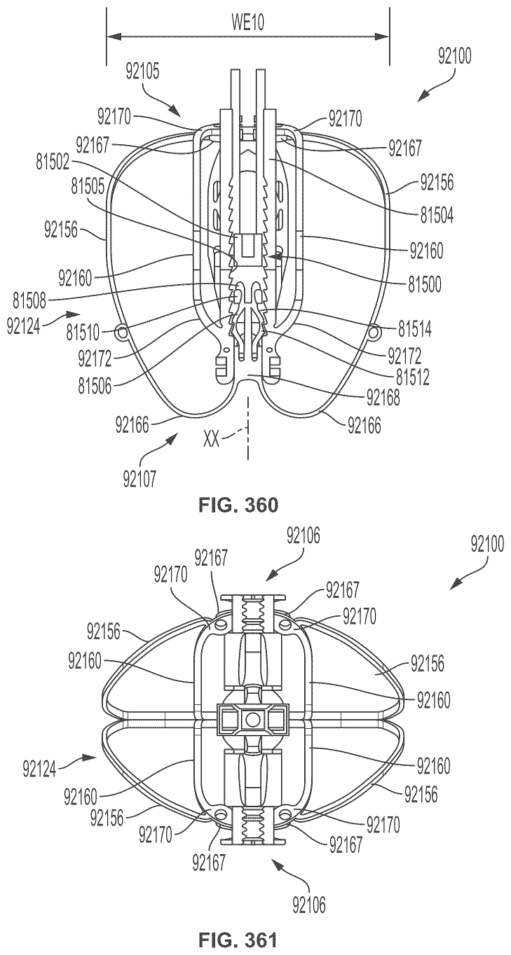

Referring to FIGS. 360-361, an example implementation of a device or implant 92100 is shown. The device or implant 92100 includes a proximal or attachment portion 92105, paddle frames 92124, an anchor portion 92106 attached to the paddle frames 92124, a width adjustment and actuation device 81500, and a distal portion 92107. The proximal portion 92105, the distal portion 92107, the width adjustment and actuation device 81500, and the paddle frames 92124 can be configured in a variety of ways.

In the illustrated example of FIGS. 360-361, the paddle frames 92124 are symmetric along longitudinal axis XX (FIG. 361). In some implementations of the device or implant 92100, however, the paddle frames 92124 is not be symmetric about the axis WW.

In the illustrated example, the paddle frames 92124 include outer frame members 92156 (e.g., struts, etc.) and inner frame members 92160 (e.g., struts, etc.). In FIGS. 360-361, the outer frame members 92156 are shown in an expanded state such that the outer frame members 92156 define a paddle frame expanded width WE10 (FIG. 360).

The outer frame members 92156 are flexibly attached to an inner end 92168 at the distal portion 92107 via connection portions 92166 and are coupled to the inner frame members 92160 at the proximal portion 92105 via connection portions 92167. Between the connection portions 92166 and the connection portions 92167, the outer frame members 92156 form a curved, convex shape. For example, in the illustrated example, the shape of the outer frame members 92156 resembles an apple shape in which the outer frame members are wider toward the proximal portion 92105 and narrower toward the distal portion 92107. In some implementations, however, the outer frame members 92156 can be otherwise shaped.

The attachment portion 92168 is configured to attach to the width adjustment and actuation device 81500 to the outer frame members 92156. The inner end 92168 can be configured in a variety of ways. Any configuration that can suitably attach the outer frame members 92156 to the width adjustment and actuation device 81500 to allow the width adjustment and actuation device 81500 to move the outer frame members 92156 between a narrowed position and an expanded position can be used.

The inner frame members 92160 are jointly attached to the outer frame members 92156 at the proximal portion 92105 via connection portions 92170 and extend from the connection portions 92170 to the distal portion 92107. The inner frame members 92160 include retaining portions 92172 near or adjacent the distal portion 92107 for attaching to the width adjustment and actuation device 81500. The retaining portions 92172 and the width adjustment and actuation device 81500 can be configured to attach in any suitable manner, such as for example, similar to how the retaining portions 91682, 91684 attach to the attachment portion 91707 of the sleeve 91702 of the device 91600.

The width adjustment and actuation device 81500 is configured to move the outer frame members 92156 from the expanded position to the narrowed position by pulling the inner end 92168 and portions of the connecting portions 92166 into the width adjustment and actuation device 81500. The width adjustment and actuation device 81500 is configured to move the inner paddle frame portions 92060 to open and close the paddles in the same or a similar manner to that shown in FIGS. 23, 27, and 30-37.

The width adjustment and actuation device 81500 includes an actuator or push/pull member 81502 (e.g., plug, slider, etc.), a receiver, such as the illustrated parallel racks 81504, and a coupling member 81506 (e.g., flexible arms, etc.). Each rack 81504 includes teeth 81505 that are configured to limit the motion of the coupling member 81506 to a single direction (e.g., a ratchet mechanism) when the coupling member is in an engaged state. In the illustrated example, the coupling member 81506 is coupled to outer paddle frames 92156 by an inner end 92168.

Still referring to FIG. 360, arms 81508 are formed on the coupling member 81506 and are configured to engage projections 81510 of the actuator or push/pull member 81502. Resilient fingers 81512 are also formed on the coupling member 81506 and are configured to engage the teeth 81505 of the rack 81504 for preventing or inhibiting the coupling member 81506 from moving along the path L in a downward or distal direction of the racks 81504.

Still referring to FIG. 360, the actuator or push/pull member 81502 can be driven in either direction along the axis XX. When the actuator or push/pull member 81502 is driven upwards, the projections 81510 of the actuator or push/pull member 81502 will pull the coupling member 81506 via the arms 81508 of the coupling member 81506. As a result, the resilient fingers 81512 will ratchet along the teeth 81505 of the rack 81504, thereby permitting the coupling member 81506 to move upwards when the actuator or push/pull member 81502 moves upwards. Simultaneously, the coupling member 81506 will cause the inner end 92168 to pull on the outer paddle frame portions 92156 and cause the outer frame portions 92156 to contract. In such examples, the position of the resilient fingers 81512 relative to each of the plurality of discrete positions (i.e., the teeth) on the rack 81504 can correspond to a particular width of the outer paddle frame portions.

Conversely, when the actuator or push/pull member 81502 is driven downwards, the projections 81510 of the actuator or push/pull member 81502 push against resilient, sloped surfaces 81514 of the coupling member 81506. As such, the projections 81510 cause the resilient fingers 81512 to disengage from the rack 81504. As such, the coupling member 81506 is disengaged from the rack 81504 when the actuator is moved in a downward or distal direction to expand the outer paddle frame portions 92156.

The paddle frames 92124 can be made from a material that allows the inner end 92168 and portions of the connecting portions 92166 to be pulled into the width adjustment and actuation device 81500. For example, the paddle frames 92124, or a portion thereof, can be made of a flexible metal, plastic, fabric, suture, etc. The paddle frames can be formed using a variety of different manufacturing processes, such as cutting, such as laser cutting, molding, forging, stamping, casting, bending, heat treating, shape setting, etc.

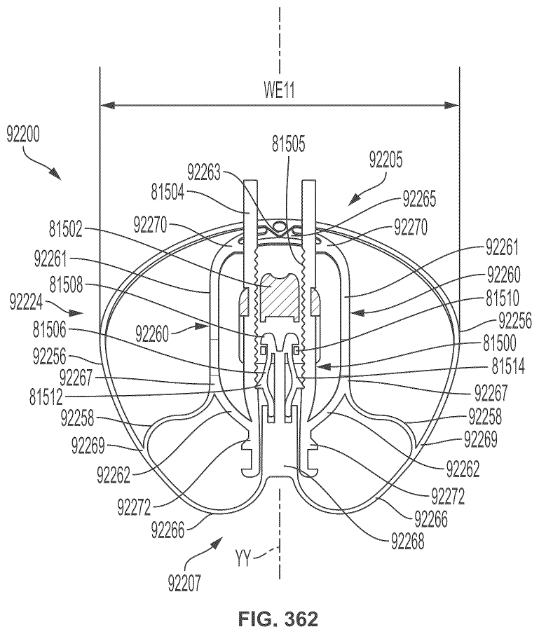

Referring to FIG. 362, an example implementation of a device or implant 92200 is shown. The device or implant 92200 includes a proximal or attachment portion 92205, paddle frames 92224, a width adjustment and actuation device 81500, and a distal portion 92207. The proximal portion 92205, the distal portion 92207, the width adjustment and actuation device 81500, and the paddle frames 92224 can be configured in a variety of ways.

In the illustrated example of FIGS. 362, the paddle frames 92224 are symmetric along longitudinal axis YY (FIG. 360). In some implementations of the device or implant 92200, however, the paddle frames 92224 are not symmetric about the axis YY.

In the illustrated example, the paddle frames 92224 includes outer frame members 92256 (e.g., struts, etc.), intermediate frame members 92258 (e.g., struts, etc.), and inner frame members 92260 (e.g., struts, etc.). In FIGS. 362, the outer frame members 92256 are shown in an expanded state such that the outer frame members 92256 define a paddle frame expanded width WE11.

The outer frame members 92256 are flexibly attached to an inner end 92268 at the distal portion 92207 via connection portions 92266 and are attached to one another at the proximal portion 92205. Between the connection portions 92266 and the proximal portion 92205, the outer frame members 92256 form a curved, convex shape. For example, in the illustrated example, the shape of the outer frame members 92256 resembles an apple shape in which the outer frame members 92256 are wider toward the proximal portion 92205 and narrower toward the distal portion 92207. In some implementations, however, the outer frame members 92256 can be otherwise shaped.

The inner end 92268 can be configured in a variety of ways. Any configuration that can suitably attach the outer frame members 92256 to the width adjustment and actuation device 81500 to allow the width adjustment and actuation device 81500 to move the outer frame members 92256 between a narrowed position and an expanded position can be used.

The inner frame members 92260 are attached to one another at the proximal portion 92205 via connection portions 92270. The outer frame members 92256 can optionally be coupled to the inner frame members 92260 at the proximal portion 92205. In the illustrated example, an inward projection 92263 of the outer frame member 92256 is disposed in a recess 92265 of the inner frame member 92260 at the proximal portion. For example, inward projection 92263 can be coupled in the recess 92265 by hinge connections, by lines or sutures, by a covering that covers the outer frame members 92056 and the inner frame members 92060, etc. The inner frame members 92260 extend along a first portion 92261 from the connection portions 92270 toward the distal portion 92207. The inner frame members 92260 then extend inward along a second portion 92262 proximate the distal portion 92207 to form retaining portions 92272 that are attached to the width adjustment and actuation device 81500. The retaining portions 92272 and the width adjustment and actuation device 81500 can be configured to attach in any suitable manner, such as for example, similar to how the retaining portions 91682, 91684 attach to the attachment portion 91707 of the sleeve 91702 of the device 91600.

The actuation and width adjustment device 81500 is configured to move the outer frame members 92256 from the expanded position to the narrowed position by pulling the inner end 92268 and portions of the connecting portions 92266 into the width adjustment and actuation device 81500. The width adjustment and actuation device 81500 is configured to move the inner paddle frame portions 92260 to open and close the paddles in the same or a similar manner to that shown in FIGS. 23, 27, and 30-37.

The width adjustment and actuation device 81500 includes an actuator or push/pull member 81502, parallel racks 81504, and a coupling member 81506. Each rack 81504 includes teeth 81505 that are configured to limit the motion of the coupling member 81506 to a single direction (e.g., a ratchet mechanism) when the coupling member is in an engaged state. In the illustrated example, the coupling member 81506 is coupled to outer paddle frames 92256 by an inner end 92268.

Still referring to FIG. 362, arms 81508 are formed on the coupling member 81506 and are configured to engage projections 81510 of the actuator or push/pull member 81502. Resilient fingers 81512 are also formed on the coupling member 81506 and are configured to engage the teeth 81505 of the rack 81504 for preventing or inhibiting the coupling member 81506 from moving along the path L in a downward or distal direction of the racks 81504.

Still referring to FIG. 362, the actuator or push/pull member 81502 can be driven in either direction along the axis YY. When the actuator or push/pull member 81502 is driven upwards, the projections 81510 of the actuator or push/pull member 81502 will pull the coupling member 81506 via the arms 81508 of the coupling member 81506. As a result, the resilient fingers 81512 will ratchet along the teeth 81505 of the rack 81504, thereby permitting the coupling member 81506 to move upwards when the actuator or push/pull member 81502 moves upwards. Simultaneously, the coupling member 81506 will cause the inner end 92268 to pull on the outer paddle frame portions 92256 and cause the outer frame portions 92256 to contract. In such examples, the position of the resilient fingers 81512 relative to each of the plurality of discrete positions (i.e., the teeth) on the rack 81504 can correspond to a particular width of the outer paddle frame portions.

Conversely, when the actuator or push/pull member 81502 is driven downwards, the projections 81510 of the actuator or push/pull member 81502 push against resilient, sloped surfaces 81514 of the coupling member 81506. As such, the projections 81510 cause the resilient fingers 81512 to disengage from the rack 81504. As such, the coupling member 81506 is disengaged from the rack 81504 when the actuator is moved in a downward or distal direction to expand the outer paddle frame portions 92260.

The intermediate frame members 92258 extend from connection portions 92267 with the inner frame members 92260 to a connection portion 92269 with the outer frame members 92256. Thus, the intermediate frame members 92258 connect the inner frame members 92260 to the outer frame members 92256 at a location between the distal portion 92207 and the proximal portion 92205. The intermediate frame members 92258 can act as strengthening struts for the device 92200 and can be configured in a variety of ways. In the illustrated example, the intermediate frame members 92258 have a wavy or S-shape. In some implementations, however, the intermediate frame members 92258 can be any suitable shape. The shape of the intermediate frame members 92258 can be selected to control the shape of the outer frame members 92256 as the outer frame members are moved between the expanded configuration and the contracted configuration.

The paddle frames 92224 can be made from a material that allows the inner end 92268 and portions of the connecting portions 92266 to be pulled into the width adjustment and actuation device 81500. For example, the paddle frames 92124, or a portion thereof, can be made of a flexible, resilient material that allows the inner end 92268 and portions of the connecting portions 92266 to be pulled repeatedly into and out of the width adjustment and actuation device 81500 without plastically deforming the inner end 92268 or the connecting portions 92266.

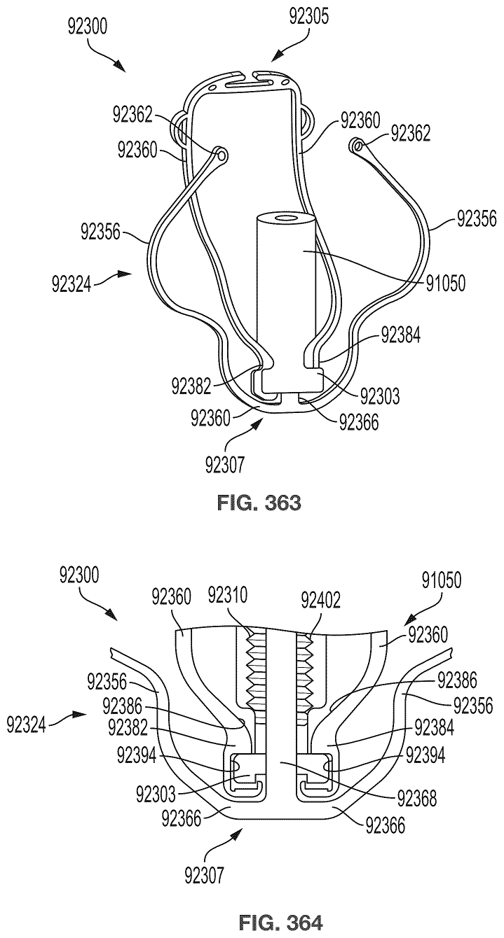

Referring to FIGS. 363-364, an example implementation of a device or implant 92300 is shown. In particular, FIGS. 363-364 illustrate an example configuration of the connection at a distal portion 92307 of the device 92300 between a width adjustment and actuation device 91050 and paddle frames 92324 of the device 92300. The distal portion 92207, the width adjustment and actuation device 91050, and the paddle frames 92324 can be configured in a variety of ways.

In the illustrated example, the paddle frames 92324 includes outer frame members 92356 and inner frame members 92360. The outer frame members 92356 are jointly attached at the distal portion 92307 by connecting portions 92366 and extend toward a proximal portion (not shown) to a terminal distal end 92362. From the connection portion 92366 to the terminal distal end 92362, the outer frame members 92356 form a curved, convex shape similar to the shape of the outer frame members 91656 of FIG. 348. In some implementations, however, the outer frame members 92356 can be shaped otherwise.

The outer frame members 92356 also include an inner end or post 92368. The post 92368 can be configured in a variety of ways, such as for example, any of the posts describe in the present application.

The inner frame members 91660 extend from a proximal portion 92305 toward the distal portion 92307. The inner frame members 92360 include a first retaining portion 92382 and a second retaining portion 92384, spaced apart from, and a mirror image of, the first retaining portion 92382. Each of the retaining portions 92382, 92384 includes an inner side surface 92386 and a recessed portion 92394 formed in the inner side surface 92386. The recessed portions 92394 are configured to engage and attach the inner frame members 92360 to a portion of the width adjustment and actuation device 91050.

The width adjustment and actuation device 91050 can be configured in a variety of ways. In the illustrated example, the width adjustment and actuation device 91050 includes a receiver, such as the illustrated internally threaded sleeve 92402, defining a through passage 92310 configured to receive the inner end or post 92368. The sleeve 92402 includes an external annular flange 92303 proximate the distal end 92307. The flange is configured to be received within the recessed portions 92394 such that the first and second retaining portions 92382, 92384 secure the inner frame members 92360 to the sleeve 92402.

In the example illustrated by FIGS. 363 and 364, the outer frame members 92356 and the inner frame members 92360 are two separate pieces. As a result, the inner end or post 92368 can be placed inside the retaining portions 92382, 92384 without having to flex the outer frame member 92356.

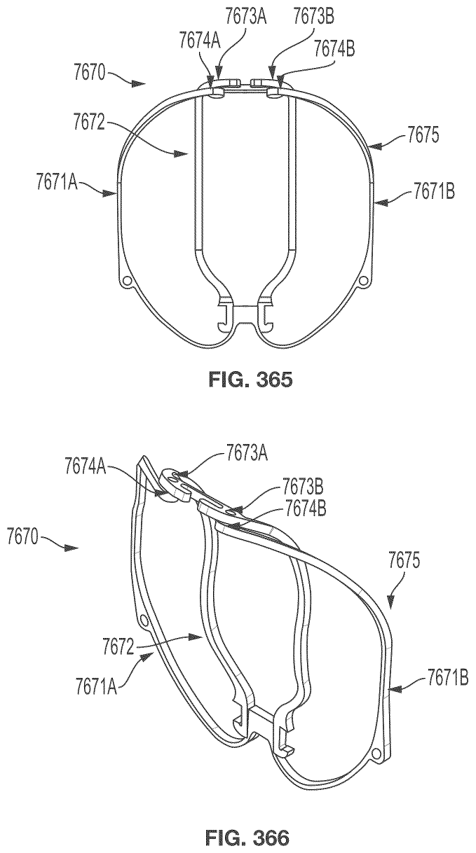

Referring to FIGS. 365 through 369, an example implementation of a connection mechanism between a rigid inner frame portion 7672 and a flexible outer frame portion 7675 of a paddle frame 7670 is shown. As shown in FIGS. 365 and 366, the proximal end of the flexible outer frame portion 7675 connects to the proximal end of the rigid inner frame portion 7672 via a pivot connection. The pivot connection can be achieved via a pin, a stitch, adhesives, or any other similar means for allowing the flexible outer frame portion 7675 to move relative to the rigid inner frame portion 7672. The proximal ends of the rigid inner frame portion 7672 and flexible outer frame portion 7675 can connect via at least one pin connection. In the example implementation, the proximal end of the rigid inner frame portion 7672 has first and second orifices 7673A, 7674B for receiving pins, stitches, or pin portions of the flexible outer frame 7675.

The flexible outer frame 7675 comprises a first portion 7671A and a second portion 7671B. The proximal end 7674A of the first portion 7671A is configured to connect to the rigid inner frame portion 7672 via the first orifice 7673A. The proximal end 7674B of the second portion 7671B is configured to connect to the rigid inner frame portion 7672 via the second orifice 7673B. These connections can comprise pin connections such that a pin (not shown) extends through the first orifice 7673A and the proximal end 7674A of the first portion 7671A of the flexible outer frame portion 7675, and a pin extends through the second orifice 7673B and the proximal end 7674B of the second portion 7671B of the flexible outer frame portion 7675. The pins can also be stitches, connector elements, or integral extensions of the proximal ends 7674A, 7674B of the first and second portions 7671A, 7671B of the flexible outer frame 7675.

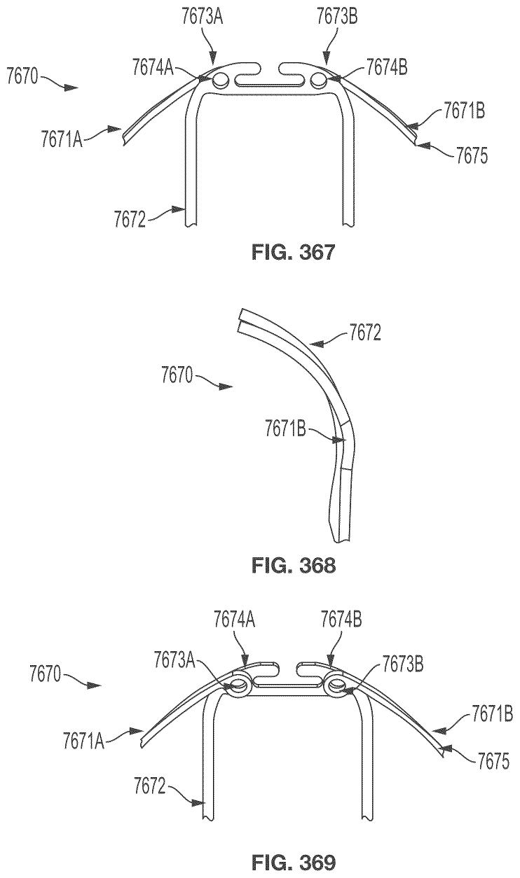

FIG. 367 shows the rigid inner frame portion 7672 having a first orifice 7673A and a second orifice 7673B. The first portion 7671A of the flexible outer frame portion 7675 connects to the rigid inner frame portion 7672 by affixing the proximal end 7674A of the flexible outer frame portion 7675 to the first orifice 7673A of the rigid inner frame portion 7672. The second portion 7671B of the flexible outer frame portion 7675 connects to the rigid inner frame portion 7672 by affixing the proximal end 7674B of the flexible outer frame portion 7675 to the second orifice 7673B of the rigid inner frame portion 7672. The proximal ends 7674A, 7674B can be affixed to the first and second portions 7671A, 7671B of the flexible outer frame portion 7675 via a pin connection, pivot connection, lamination, or any other means. The flexible outer frame portion 7675 of the paddle frame 7670 can be on top of the rigid inner frame portion 7672, as shown in FIG. 367. Alternatively, as shown in FIG. 369, the flexible outer frame portion 7675 can be below the rigid inner frame portion 7672. FIG. 368 shows a partial side view of this example of the paddle frame 7670.

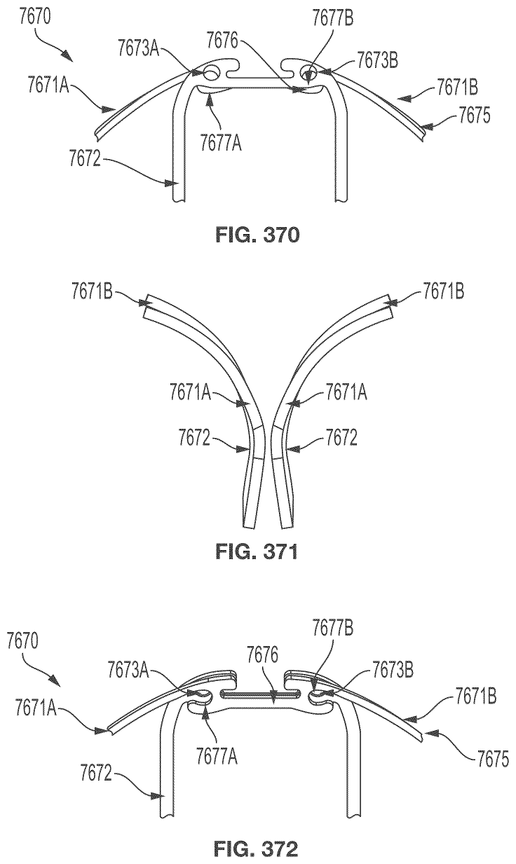

Referring to FIGS. 370 through 372, an example of a connection mechanism between a rigid inner frame portion 7672 and a flexible outer frame portion 7675 of a paddle frame 7670 is shown. As shown in FIGS. 370 and 372, the proximal end 7676 of the flexible outer frame portion 7675 can be laminated, or otherwise affixed, to the proximal end of the rigid inner frame portion 7672. The inner and outer frame portions 7672, 7675 can also or instead be pivotably connected via a first and second pivot point 7677A, 7677B. The pivot connection allows the flexible outer frame portion 7675 to bend relative to the rigid inner frame portion 7672. The pivot connection can be achieved via a pin, a stitch, connection element, adhesives, or any other similar means for allowing the flexible outer frame portion 7675 to move relative to the rigid inner frame portion 7672. In this example, the pivot connection is such that the flexible outer frame portion 7675 is on the outside of the rigid inner frame portion 7672. However, as shown in FIG. 372, the flexible outer frame portion 7675 could also be on the inside of the rigid inner frame portion 7672.

The proximal ends of the rigid inner frame portion 7672 and flexible outer frame portion 7675 can connect via at least one pivot. In some implementations, the proximal end of the rigid inner frame portion 7672 has a first and second orifice 7673A, 7673B for receiving a pin, a stitch, connection element, or pin portions of the flexible outer frame 7675. The flexible outer frame 7675 comprises a first portion 7671A and a second portion 7671B. A first pivot point 7677A of the first portion 7671A is configured to connect to the rigid inner frame portion 7672 via the first orifice 7673A. The second pivot point 7677B of the second portion 7671B is configured to connect to the rigid inner frame portion 7672 via the second orifice 7673B. These connections can comprise pin connections such that a pin (not shown) extends through the first orifice 7673A and the proximal end 7674A of the first portion 7671A of the flexible outer frame portion 7675, and a pin extends through the second orifice 7673B and the proximal end 7674B of the second portion 7671B of the flexible outer frame portion 7675. The pins can also be integral extensions of the proximal ends 7674A, 7674B of the first and second portions 7671A, 7671B of the flexible outer frame 7675. FIG. 371 shows a partial side view of this example of the paddle frame 7670.

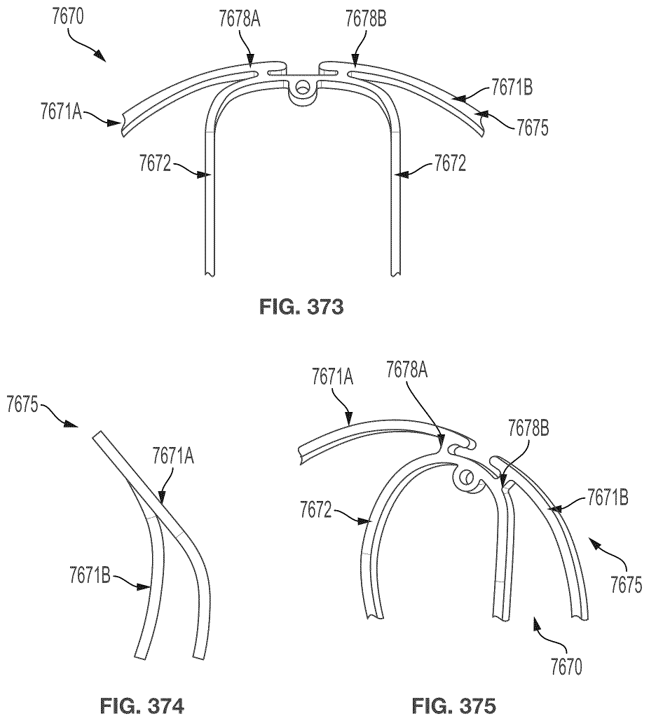

Referring to FIGS. 373-375, an example of a connection mechanism between a rigid inner frame portion 7672 and a flexible outer frame portion 7675 of a paddle frame 7670 is shown. The proximal ends of the inner and outer frame portions 7672, 7675 of the paddle frame 7670 are integrally joined together. The connection can be formed from one single piece having a first pivot point 7678A and a second pivot point 7678B. The first pivot point 7678A is located at the integration point between a first portion 7671A of the flexible outer frame portion 7675 with the rigid inner frame portion 7672. The second pivot point 7678B is formed from the integration point between the second portion 7671B of the flexible outer frame portion 7675 and the rigid inner frame portion 7672. Although the flexible outer frame portion 7675 and rigid inner frame portion 7672 are formed with one piece, the flexible outer frame portion 7675 can flex relative to the rigid inner frame portion 7672 via the first and second pivot points 7678A, 7678B.

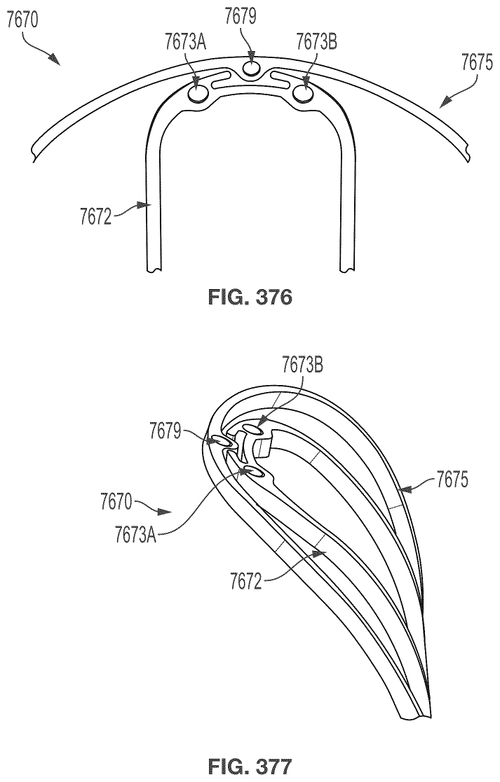

Referring to FIGS. 376 and 377, an example of a connection mechanism between a rigid inner frame portion 7672 and a flexible outer frame portion 7675 of a paddle frame 7670 is shown. In this example, the proximal ends of the outer and inner frame portions 7672, 7675 of the paddle frame 7670 are nested together. The flexible outer frame portion 7675 can have an orifice 7679 proximate to a first and second orifice 7673A, 7673B within the proximal end of the rigid inner frame portion 7672. The inner and outer frame portions 7672, 7675 of the paddle frame 7670 can be connected by any means, including but not limited to: nesting together within the cover 240, stitched together via the orifices 7679, 7673A, 7673B, or connected via connectors (not shown) that extend between the first and second orifices 7673A, 7673B and the orifice 7679 on the flexible outer frame portion 7675.

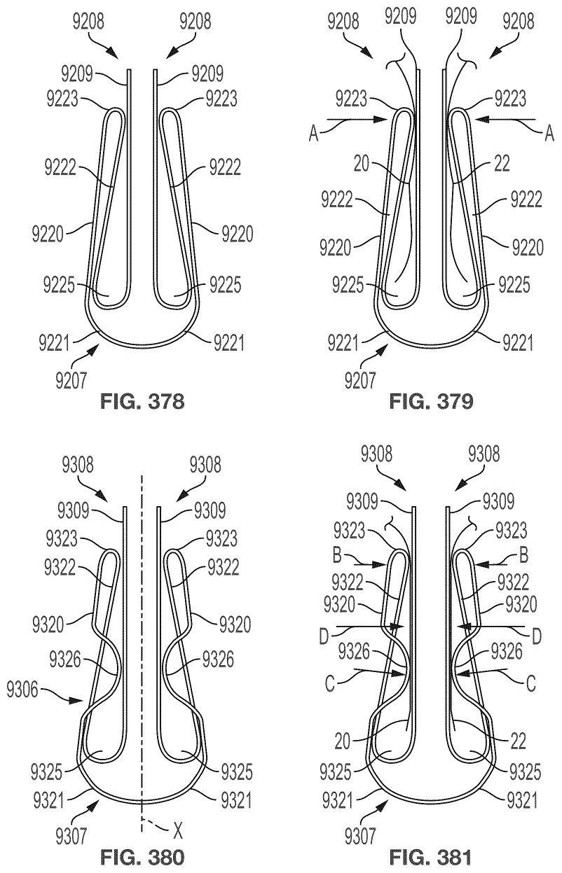

Referring to FIGS. 378-379, anchors 9208 for a device or implant are schematically illustrated, such as for example, the anchors 208 for the example device or implant 200 of FIGS. 22-27. The anchors 9208 are illustrated in a closed position. The anchors 9208 include inner members 9209, inner paddles 9222, and outer paddles 9220.

The inner members 9209 can be a portion of a coaptation element, such as coaptation element 210 of FIGS. 22-27, or attached to a coaptation element by any suitable means. The outer paddles 9220 are flexibly attached at a distal portion 9207 by connection portions 9221 and to the inner paddles 9222 by connection portions 9223. The inner paddles 9222 are flexibly attached to the inner members 9209 by connection portions 9225. In this manner, the anchors 9208 are configured similar to legs in that the inner paddles 9222 are like upper portions of the legs, the outer paddles 9220 are like lower portions of the legs, and the connection portions 9223 are like knee portions of the legs.

As shown in FIG. 379, due to the configuration of the anchors 9208 and the coaptation element, such as coaptation element 210 of FIGS. 22-27, when the anchors 9208 are closed onto native valve leaflets 20, 22, each of the leaflets 20, 22 is secured between a corresponding one of the inner paddles 9222 and one of the inner members 9209 at a single position, or engagement region, near or adjacent the connection portion 9223, as shown by arrows A.

Referring to FIGS. 380-383, an example implementation of anchors 9308 for a device or implant is schematically illustrated in the closed position. The anchors 9308 are configured such that when the anchors 9308 are closed onto the native valve leaflets 20, 22, the anchors 9308 secure each of the native valve leaflets 20, 22 at more than a single position or single engagement region. To achieve this, the anchors 9308 can be configured in a variety of ways.

In the illustrated example, the anchors 9308 include inner members 9309 (e.g., flat metal posts, etc.), inner paddles 9322 (e.g., flat metal strip, etc.), and outer paddles 9320 (e.g., flat metal strip, etc.). The inner members 9309 can be formed integrally with a coaptation element, such as the coaptation element 210 of FIGS. 22-27, or can be attached to a coaptation element by any suitable means. The outer paddles 9320 are flexibly attached at a distal portion 9307 by connection portions 9321 and to the inner paddles 9322 by connection portions 9323. The inner paddles 9322 are flexibly attached to the inner members 9309 by connection portions 9325. In this manner, the anchors 9308 are configured similar to legs in that the inner paddles 9322 are like upper portions of the legs, the outer paddles 9320 are like lower portions of the legs, and the connection portions 9323 are like knee portions of the legs.

In the illustrated example, the anchors 9308 are integrally formed together as a single anchor portion 9306 that is symmetric about a longitudinal axis X. In some implementations, however, the anchors 9308 are not formed as a single integrated structure and/or the structure is not symmetric.

Unlike the example of FIGS. 378-379, the outer paddles 9320 of the anchors 9308 include one or more inward biasing portions 9326. The one or more inward biasing portions 9326 can be configured in a variety of ways. Any portion that can act to secure a native valve leaflet in the anchor 9308 at a position between the connection portion 9323 and the connection portion 9325, in addition to, or as an alternative to, the point adjacent the connection portion 9323, can be used. In some implementations, the entire anchor 9308 or portions of the anchor 9308, such as the inward biasing portions 9326 can be made from a shape-memory material-such as Nitinol—to provide shape-setting capability.

In the illustrated example, each of the outer paddles 9320 includes an inward biasing portion 9326 in the form of a concave or inwardly curved portion positioned along the outer paddles 9320. In some implementations, however, the inward biasing portions 9326 can have shapes other than inwardly curved.

As shown in FIGS. 380-381, the inward biasing portions 9326 can extend inward beyond the inner paddles 9322. For example, each of the inward biasing portions 9326 can be configured to be received through a corresponding aperture 9328 in the inner paddles 9322 (see FIG. 384) or can be configured to extend around the inner paddles 9322 such that the inward biasing portions 9326 can act to secure the native valve leaflets 20, 22 in the anchors 9308.

As shown by arrows B and C in FIG. 381, the anchors 9308 are configured to secure each of the native valve leaflets 20, 22 within the anchors 9308 at two separate and longitudinally spaced apart locations, or engagement regions, between the inner member 9309, or coaptation element, and the inner paddles 9322. The first location or engagement region B is near or adjacent the connection portion 9323 while the second location or engagement region C is between the first location B and the connection portion 9325. As shown in FIG. 381, the first location or engagement region B can be separated from the second location or engagement region C by a non-engagement region D (FIG. 381) where the leaflet is not being pinched or secured.

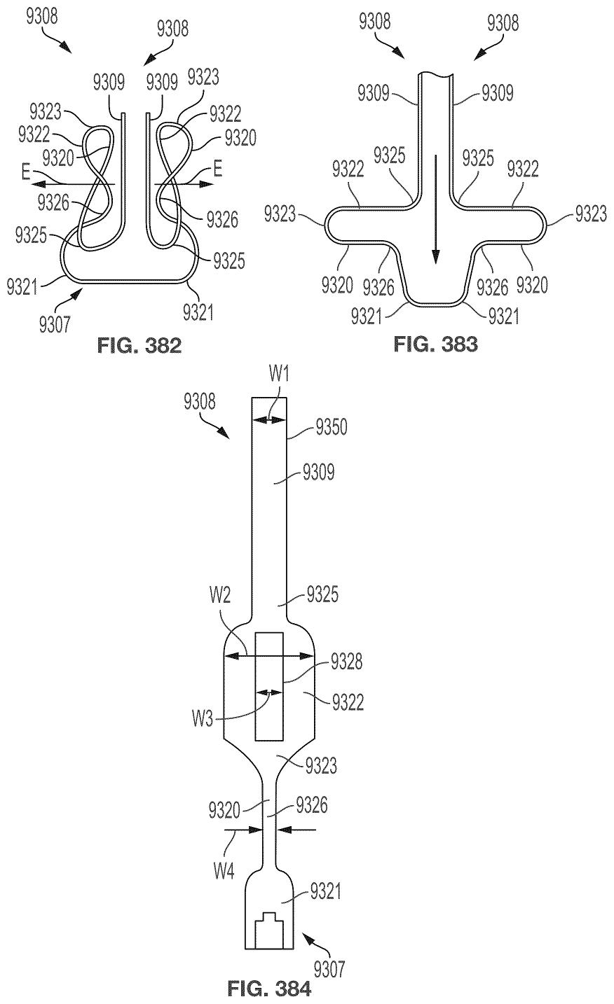

Referring to FIGS. 382 and 383, a schematic representation of the anchors 9308 in a partially open position and in a fully open position are shown, respectively. As shown in FIG. 382, as the anchors 9308 move from the closed position toward an open position, the inner paddles 9322 pivot, flex, and/or articulate outward at the connection portions 9325, as shown by arrows E in FIG. 382. In other words, the angle between the inner paddles 9322 and the inner members 9309 increase. As the inner paddles 9322 pivot, flex, and/or articulate outward, the outer paddles 9320 follow.

As shown in FIG. 383, in the open position, the inner paddles 9322 extend outward from the inner members 9309. In the illustrated example, the inner paddles 9322 are at, or near, perpendicular to the inner members 9309 (i.e., 90 degrees relative to the inner member). In some implementations, however, in the open position, the inner paddles 9322 can extend relative to the inner members 9309 at an angle greater than or less than 90 degrees.

In the open position, the inward biasing portions 9326 of the outer paddles 9320 form a concave shape between the connection portions 9323 and the connection portions 9321.

Referring to FIG. 384, one half of an example implementation of the anchor portion 9306 is shown (i.e., one inner member 9309, one inner paddle 9322, and one outer paddle 9320). In the illustrated example, the inner member 9309 is a generally rectangular strip having generally parallel side edges 9350 and a width W1.

In the illustrated example, the inner paddle 9322 has a width W2 that is greater than the width W1 of the inner member 9309. In some implementations, however, the width W2 of the inner paddle 9322 can be equal to or less than the width W1 of the inner member 9309. The width W2 of the inner paddle 9322 is wide enough to allow a fixed arm of the clasp to be mounted thereto, as described below. In the illustrated example, the inner paddle 9322 includes an aperture 9328, such as a slot, having a width W3 and being configured to receive the inward biasing portion 9326 of the outer paddle 9320 therethrough.

The outer paddle 9320, in the illustrated example, is a relatively thin strip, as compared to the inner paddle 9322, and has a width W4. The width W4 is smaller than the width W3 of the aperture 9328 so that at least the inward biasing portion 9326 of the outer paddle 9320 can be received through the aperture 9328. In some implementations, the inward biasing portion 9326 of the outer paddle 9320 has a width W4 that is less than the width W3 of the aperture 9328 while other portions of the outer paddle 9320 can have widths greater than or less than the width W4.

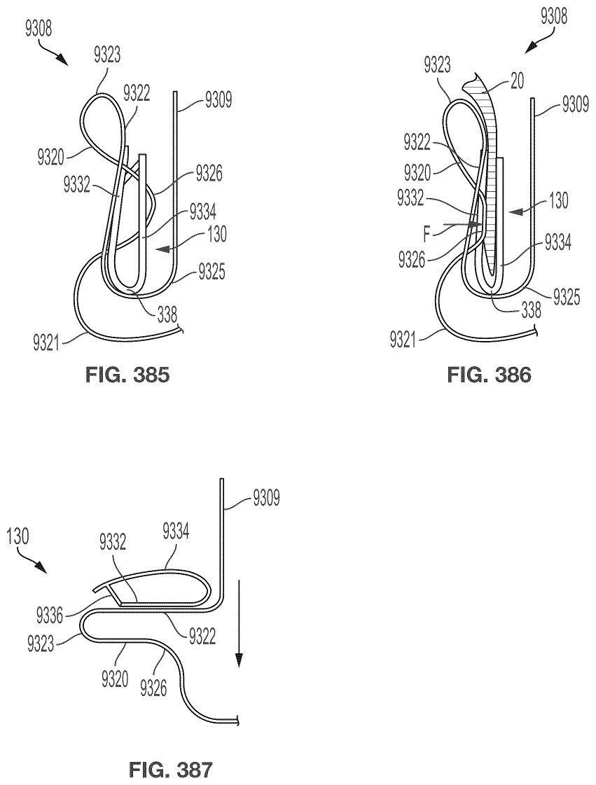

Referring to FIGS. 385-386, one of the anchors 9308 is schematically illustrated in the closed position with an attachment portion or gripping member installed. The gripping member is illustrated as a clasp 130 that includes a base or fixed arm 9332, a moveable arm 9334, and a joint portion 338. The clasp 130 is shown in a closed position in which the moveable arm 9334 and the fixed arm 9332 are adjacent, or near each other, such that the clasp 130 resembles a U-shape.

As shown in FIG. 385, in the closed position, the anchor 9308 is configured such that the inward biasing portion 9326, in a free state in which a native valve leaflet is not captured in the clasp 130, extends inward past the inner paddle 9322 and past the fixed arm 9332 and optionally past the moveable arm 9334 of the clasp 130. The clasp 130, therefore, is configured to allow inward biasing portion 9326 to extend through, or around, the fixed arm 9332 and the moveable arm 9334. For example, as in more detail discussion below, the fixed arm 9332 and the moveable arm 9334 can include apertures that allow the inward biasing portion 9326 of the outer paddle 9320 to extend therethrough.

Referring to FIG. 386, the anchor 9308 of FIG. 385 is illustrated with a native valve leaflet 20 captured within the clasp 130. The leaflet 20 is received between the fixed arm 9332 and the moveable arm 9334 of the clasp 130. The inward biasing portion 9326 of the outer paddle 9320 extends past or through the inner paddle 9322 and the fixed arm 9332 to engage the leaflet 20 and bias the leaflet 20 inward toward, the movable arm 9334 of the clasp. The position of the inward biasing portion 9326 in the free state (FIG. 385) illustrates that when the valve leaflet 20 is received in the clasp 130, as shown in FIG. 386, the inward biasing portion 9326 is forced outward against its bias. As a result, the inward biasing portion 9326 is exerting an inward force, as shown by arrow F in FIGS. 386, against the valve leaflet 20.

Referring to FIG. 387, the anchor 9308 is schematically illustrated in the open position with the clasp 130 attached to the anchor 9308 and in a closed position. The clasp 130 of FIG. 387 optionally includes one or more securing elements 9336 (e.g., barbs, ridges, points, rough surfaces, etc.). The securing elements 9336 can be configured in a variety of ways. For example, the securing elements 9336 can include elements that pierce or indent into the leaflet of a native valve, such as for example barbs or other projections, or can include friction-enhancing elements that increase the friction between the clasp 130 and the leaflet to secure the leaflet in place. The friction-enhancing elements can be configured to secure the leaflet in place without piercing the leaflet.

As shown in FIG. 387, the fixed arm 9332 of the clasp 130 is attached to the inner paddle 9322 such that the fixed arm 9332 moves with the inner paddle 9322 while the moveable arm 9334 remains adjacent, or near the fixed arm 9332 such that the clasp 130 resemble a U-shape. The fixed arm 9332 can be attached to the inner paddle 9322 in any suitable manner.

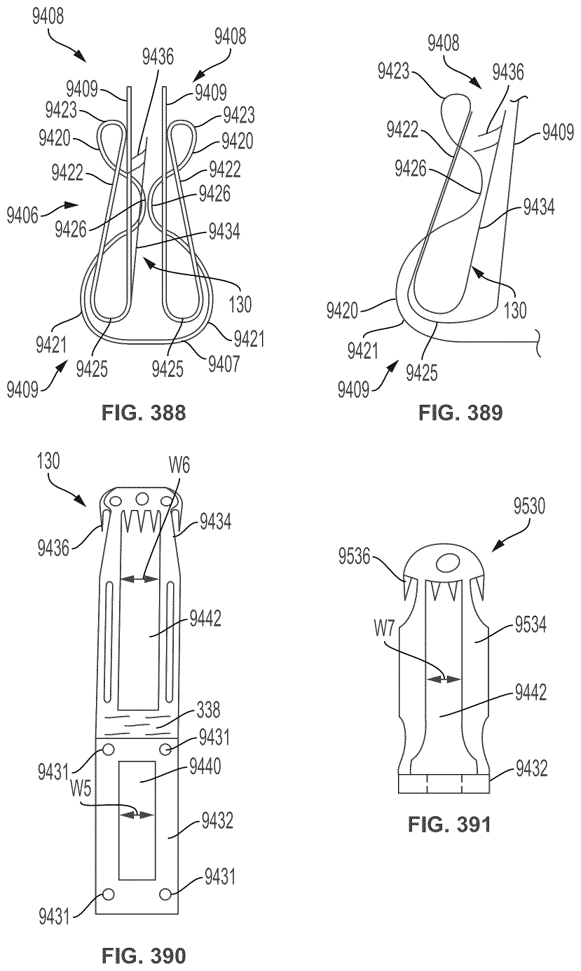

Referring to FIGS. 388-389, a schematic illustration of an example implementation of an anchor portion 9406 having two anchors 9408 for a device or implant are schematically illustrated in the closed position. The anchors 9408 include inner members 9409, inner paddles 9422, and outer paddles 9420. The inner members 9409 can be a portion of a coaptation element, such as a coaptation element 210 of FIGS. 22-27, or attached to a coaptation element by any suitable means. The outer paddles 9420 are flexibly attached at a distal portion 9407 by connection portions 9421 and to the inner paddles 9422 by connection portions 9423. The inner paddles 9422 are flexibly attached to the inner members 9409 by connection portions 9425. In this manner, the anchors 9408 are configured similar to legs in that the inner paddles 9422 are like upper portions of the legs, the outer paddles 9420 are like lower portions of the legs, and the connection portions 9423 are like knee portions of the legs.

The outer paddles 9420 of the anchors 9408 include one or more inward biasing portions 9426, such as for example, similar to the inward biasing portions 9326 described regarding the example of FIGS. 380-382.

In the illustrated example, each of the outer paddles 9420 includes an inward biasing portions 9426 in the form of a concave or inwardly curved portion positioned along the outer paddles 9420 at a location closer to the connection portion 9423 than to the connection portion 9421. In some implementations, however, the inward biasing portions 9426 can have shapes other than inwardly curved and can be positioned at a mid-point between the connection portion 9423 and the connection portion 9421 or closer to the connection portion 9421.

As shown in FIGS. 388-389, the inward biasing portions 9426 can extend inward beyond the inner paddles 9422 and beyond the inner members 9409. For example, each of the inward biasing portions 9426 can be configured to be received through a corresponding aperture (not shown) in the inner paddles 9422 or can be configured to extend around the inner paddles 9422. The aperture (not shown) in inner paddles 9422 can be similar, for example, as described regarding aperture 9328 of the example of FIG. 384.

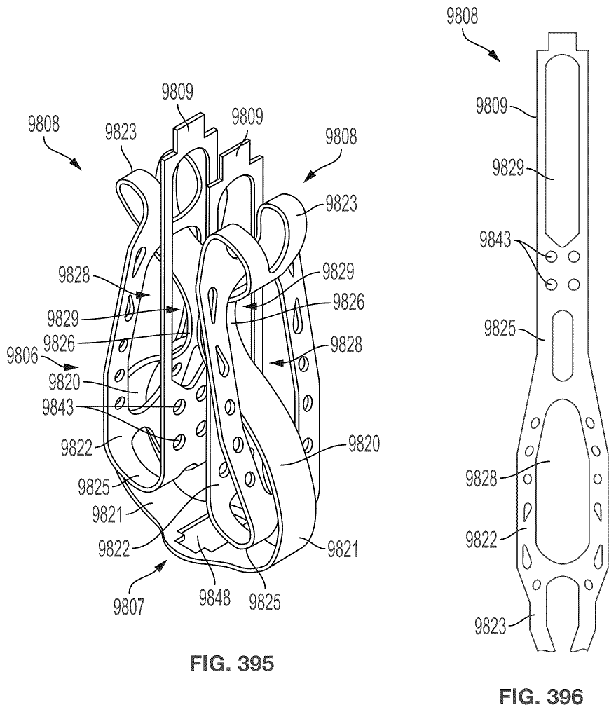

Similarly, the inward biasing portions 9426 and the inner members 9409 can be configured such that the inward biasing portions 9426 can be received through a corresponding aperture, such as aperture 9828 of FIG. 395, in the inner member 9409 or can be configured to extend around the inner member 9409.

A clasp 130 having a movable arm 9434 can be attached to the anchors 9408. The moveable arm 9434 of clasp 130 can be similar as described regarding the moveable arm 9334 of clasp 130 of FIGS. 385-387. FIG. 388 illustrates the inward biasing portion 9426 can extend through, or past, the inner member 9409 and the moveable arm 9434, while the moveable arm 9434 can also extend through, or past, the inner member 9409. Thus, the clasp 130 can be configured to allow the inward biasing portion 9426 to extend through or around it and the inner member 9409 can be configured to allow both the inward biasing portion 9426 and the moveable arm 9434 to extend through it.

The configuration of FIGS. 388-389 allows the anchors 9408 to be configured such that the inward biasing portions 9426 provide a larger inward biasing force than would be achievable with the inward biasing portions 9426 not extending through the inner members 9409 in their free states. It should be appreciated, however, that the inner members 9409 are typically attached to a coaptation element, such as for example, the coaptation element 210 of FIGS. 22-27. Thus, for the assembled device or implant, the moveable arm 9434 of the clasp 130 and the inward biasing portion 9426 will be blocked by the coaptation element 210 from extending inward as far as illustrated in FIG. 388. FIG. 388 illustrates a degree of inward bias the inward biasing portion 9426 can be initially shape-set to.

FIG. 389 shows a similar example to that of FIG. 388 with the anchor 9408 with inward biasing portion 9426 not extended through the moveable arm 9434 of the clasp or the inner member 9409.

Referring to FIG. 390, a plan view of an example implementation of the clasp 130 for a device or implant is shown with the clasp 130 in a laid-open position. The clasp 130 can be configured and operate similar to the clasp 130 described above. For example, the clasp 130 includes a base or fixed arm 9432, the moveable arm 9434, securing elements 9436, and a joint portion 338. The fixed arms 9432 are configured to be attached to the inner paddles 9422 of the anchors 9408 through holes or slots 9431 with sutures (not shown). The fixed arms 9432 remain stationary relative to the inner paddles 9422 when the moveable arms 9434 are opened to expose the securing elements 9436.

The joint portion 338 connects the fixed arm 9432 to the moveable arm 9434. The joint portion 338 provides a spring force between the fixed and moveable arms 9432, 9434. The joint portion 338 can be any suitable joint, such as a flexible joint, a spring joint, a pivot joint, or the like. In some implementations, the joint portion 338 is a flexible piece of material integrally formed with the fixed and moveable arms 9432, 9434.

As shown in FIG. 390, in the illustrated example, the fixed arm 9432 has an aperture 9440 and the moveable arm 9434 has an aperture 9442 for receiving the inward biasing portion 9426 therethrough. The aperture 9440 of the fixed arm 9432 has a width W5 and the aperture 9442 of the moveable arm 9434 has a width W6. The width W5 and the width W6 are wider than the width of the inward biasing portion 9426.

Referring to FIG. 391, a view of an example implementation of an open clasp 9530 shows the moveable arm 9534 of the clasp 9530. The moveable arm 9534 includes securing elements 9536 and an aperture 9442 for receiving the inward biasing portion 9426 therethrough. The aperture 9442 of the moveable arm 9534 has a width W7 wider than the width of the inward biasing portion 9426.

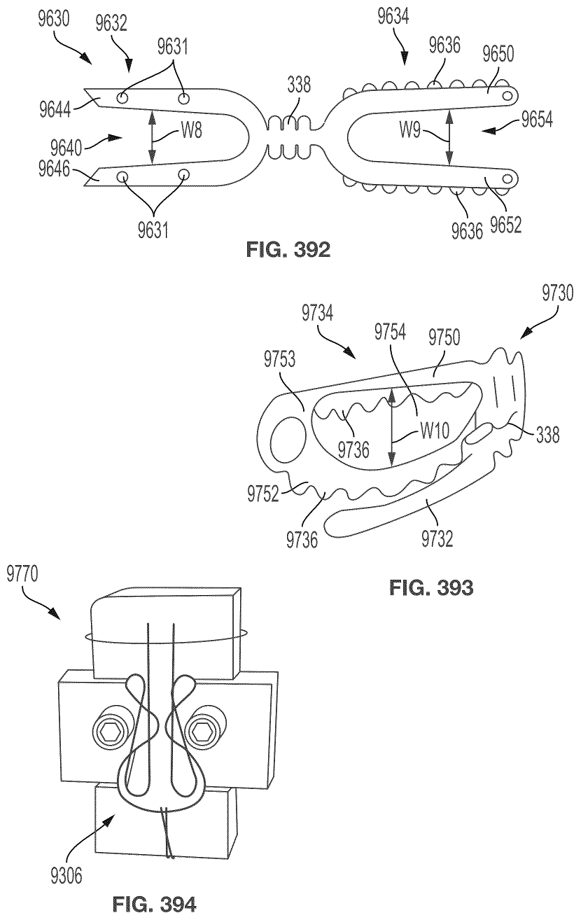

Referring to FIG. 392, a plan view of an example implementation of a clasp 9630 for a device or implant is shown with the clasp 9630 laid open. The clasp 9630 can be configured and operate similar to the clasp 130 described above. For example, the clasp 9630 includes a base or fixed arm 9632, a moveable arm 9634, securing elements 9636, and a joint portion 338.

In the illustrated example, the fixed arm 9632 and the moveable arm 9634 are bifurcated and open-ended. In particular, the fixed arm 9632 includes a first fixed arm portion 9644 and a second fixed arm portion 9646 separated by an open area 9640 such that the fixed arm 9632 resembles a U-shape. In one example, the first fixed arm portion 9644 is parallel to the second fixed arm portion 9646. In some implementations, however, the first fixed arm portion 9644 is not parallel to the second fixed arm portion 9646. The open area 9640 has a width W8 that is wider than the width of an inward biasing portion of an anchor, such as for example, the width of the inward biasing portion 9426 of the anchor 9408, to allow the inward biasing portion to be received through the open area 9640.

The fixed arm 9632 is configured to be attached to an inner paddle of an anchor, such as for example, the inner paddle 9422 of the anchor 9408, through holes or slots 9631 with sutures (not shown).

Similar to the fixed arm 9632, the moveable arm 9634 includes a first moveable arm portion 9650 and a second moveable arm portion 9652 separated by an open area 9654 such that the moveable arm 9634 resembles a U-shape. In some implementations, the first moveable arm portion 9650 is parallel to the second moveable arm portion 9652. In some implementations, however, the first moveable arm portion 9650 is not parallel to the second moveable arm portion 9652. The open area 9654 has a width W9 that is wider than the width of an inward biasing portion of an anchor, such as for example, the width of the inward biasing portion 9426 of the anchor 9408, to allow the inward biasing portion to be received through the open area 9654.

The joint portion 338 connects the fixed arm 9632 to the moveable arm 9634. The joint portion 338 provides a spring force between the fixed and moveable arms 9632, 9634. The joint portion 338 can be any suitable joint, such as a flexible joint, a spring joint, a pivot joint, or the like. In some implementations, the joint portion 338 is a flexible piece of material integrally formed with the fixed and moveable arms 9632, 9634.

The securing elements 9636 can be configured in a variety of ways. In the illustrated example, the securing elements 9636 are friction-enhancing elements that provide a secure grip on the leaflet of a native valve when the leaflet is between the fixed arm 9632 and the moveable arm 9634 when the clasp 9630 is in a closed position. The friction-enhancing elements can be configured to secure the leaflet in place without piercing the leaflet. The friction-enhancing elements 9636 can be any suitable elements that facilitate or provide for a secure grip on the leaflet. For example, the friction-enhancing elements 9636 can provide a rough surface area, such as a knurled, bumpy, or coarse portion. The friction-enhancing elements can include one or more materials that enhance friction. The number of, the size and shape of, and location of the friction-enhancing elements 9636 can vary in some implementations. Any suitable number, size, shape, and location that facilitates securely gripping a native valve leaflet within the closed clasp can be used.

Referring to FIG. 393, a side plan view of an example implementation of a clasp 9730 for a device or implant with the clasp 9730 in a closed position is shown. The clasp 9730 can be configured and operate similar to the clasp 9630 described above. For example, the clasp 9730 includes a base or fixed arm 9732, a moveable arm 9734, friction-enhancing elements 9736 or other securing elements, and a joint portion 338.

Unlike the bifurcated and open-ended moveable arm 9634 of the example of FIG. 392, the moveable arm 9734 of the clasp 9730 is closed-ended. In particular, the moveable arm 9734 includes a first moveable arm portion 9750 and a second moveable arm portion 9752. In one example, the first moveable arm portion 9750 is parallel to the second moveable arm portion 9752. In some implementations, however, the first moveable arm portion 9750 is not parallel to the second moveable arm portion 9752.

The first moveable arm portion 9750 and a second moveable arm portion 9752 are connected via an optional bridge portion 9753 on the moveable arm 9734 distal from the joint portion 338. Thus, the first moveable arm portion 9750, the second moveable arm portion 9752, and the bridge portion 9753 form a closed aperture 9754 having a width W10 that is wider than the width of an inward biasing portion of an anchor, such as for example, the width of the inward biasing portion 9426 of the anchor 9408, to allow the inward biasing portion to be received through the aperture 9754.

Referring to FIG. 394, the anchor portion 9306 is shown in a shape memory alloy shape-setting jig 9770. As discussed above, the anchor portion 9306, or select portions thereof, can be made from shape-memory alloy material—such as Nitinol—to provide shape-setting capability. Thus, the anchor 9308 can be secured in a shape memory alloy shape-setting jig 9770, such as shown in FIG. 394. Methods for shape-setting shape memory alloy materials are well known in the art and will not be discussed in detail in this application. The anchor portion 9308 can be shape-set in a shape memory alloy shape-setting jig 9770 in any suitable methods, such as conventional shape setting methods.

Referring to FIG. 395-396, an example implementation of an anchor portion 9806 for a device or implant is shown in a closed position. FIG. 395 shows the shaped anchor portion and FIG. 396 shows a portion of the anchor portion in the flat (e.g., before it is shaped). The anchor portion 9806 includes a pair of anchors 9808 configured such that when the anchors 9808 are closed onto the native valve leaflets, the anchors 9808 secure each of the native valve leaflets in more than a single location.

In the illustrated example, the anchors 9808 includes inner members 9809 (e.g., flat metal posts, etc.), inner paddles 9822 (e.g., strips, etc.), and outer paddles 9820 (e.g., strips, etc.). The inner members 9809 can be a portion of a coaptation element, such as a coaptation element 210 of FIGS. 22-27, or attached to a coaptation element by any suitable means. The outer paddles 9820 are jointably attached at a distal portion 9807 by connection portions 9821 and to the inner paddles 9822 by connection portions 9823. The inner paddles 9822 are flexibly attached to the inner members 9809 by connection portions 9825. In this manner, the anchors 9808 are configured similar to legs in that the inner paddles 9822 are like upper portions of the legs, the outer paddles 9820 are like lower portions of the legs, and the connection portions 9823 are like knee portions of the legs.

In the illustrated example, the anchors 9808 are integrally formed together as a single anchor portion 9806 that symmetric about a longitudinal axis. In some implementations, however, the anchors 9808 is not formed as a single integrated structure and/or the structure is not symmetric.

The outer paddles 9820 include one or more inward biasing portions 9826. The one or more inward biasing portions 9826 can be configured in a variety of ways. Any portion that can act to secure a native valve leaflet in the anchor at a position between the connection portions 9823 and the connection portions 9825 can be used. In some implementations, the anchors 9808 in their entirety, or select portions of the anchors 9808, such as the inward biasing portions 9826, can be made from shape-memory alloy material—such as Nitinol—to provide shape-setting capability.

In the illustrated example, the inward biasing portions 9826 are concave or inwardly curved portions. In some implementations, however, the inward biasing portions 9826 can have shapes other than inwardly curved. As shown in FIG. 395, the inward biasing portions 9826 extend inward beyond the inner paddles 9822 and the inner members 9809. For example, each of the inward biasing portions 9826 can be configured to be received through a corresponding aperture 9828 in the inner paddles 9822 and through a corresponding aperture 9829 in the inner members 9809.

The anchor portion 9806 includes a mounting aperture 9848 for attaching the anchor portion 9806 to a cap (not shown) at the distal portion 9807, such as for example, the cap 214 of the examples of the device of FIG. 22-27. The inner member 9809 can include one or more holes or slots 9843 for attaching the inner member 9809 to a coaptation element, such as for example, the coaptation element 210 of FIGS. 22-27.

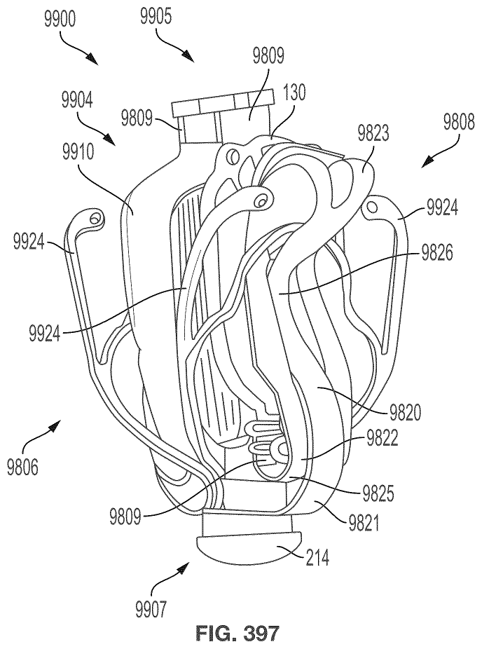

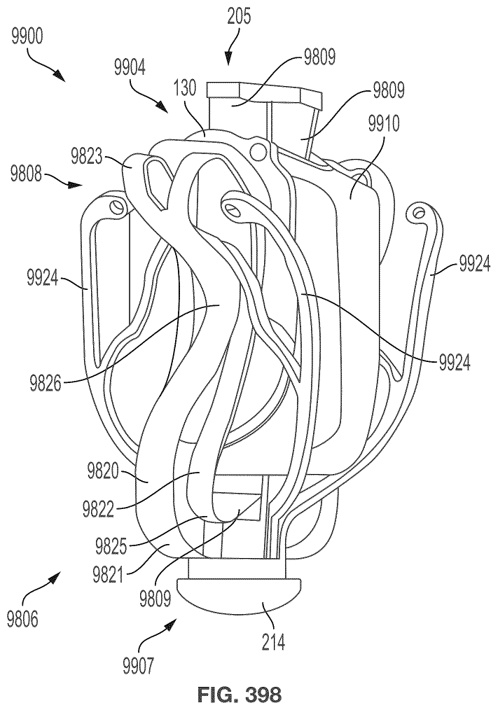

Referring to FIGS. 397-399, an example implementation of the device or implant 9900 is shown. The device 9900 is one of the many different configurations that the device 9900 can take. The device 9900 can include any features of devices or implants discussed elsewhere in the present application, and the device 9900 can be positioned to engage valve tissue as part of any suitable valve repair system (e.g., any valve repair system disclosed in the present application).

The device or implant 9900 includes a coaptation portion or coaptation portion 9904, a proximal or attachment portion 9905, an anchor portion, such as anchor portion 9806 of FIG. 395, and a distal portion 9907. In some implementations, the coaptation portion 9904 of the device 9900 includes a coaptation element 9910 for implantation between the leaflets of the native valve.

As discussed with respect to FIG. 395, the anchor portion 9806 includes anchors 9808 having inner members 9809 (FIGS. 397-398), inner paddles 9822, and outer paddles 9820. The inner members 9809 are attached to a coaptation element 9910. The outer paddles 9820 are flexibly attached at the distal portion 9907 by connection portions 9821 and to the inner paddles 9822 by connection portions 9823. The inner paddles 9822 are flexibly attached to the inner members 9809 by connection portions 9825.

The device also includes clasps 130 and paddle extension members or paddle frames 9924. The clasps 130 can include any of the features of the clasps discussed in the present application, such as for example, the clasp 130 of FIG. 390. The paddle frames 9924 can include any of the features of the paddle frames discussed in the present application, such as for example, paddle frames 224 of FIGS. 22-27. The paddle frames 9924 are attached to a cap 214 at the distal portion 9907 and extend to the connection portions 9823 between the inner and outer paddles 9822, 9820.

The outer paddles 9820 include the inward biasing portions 9826 in the form of concave or inwardly curved portions positioned along the outer paddles 9820 between the connection portion 9823 and the connection portion 9821.

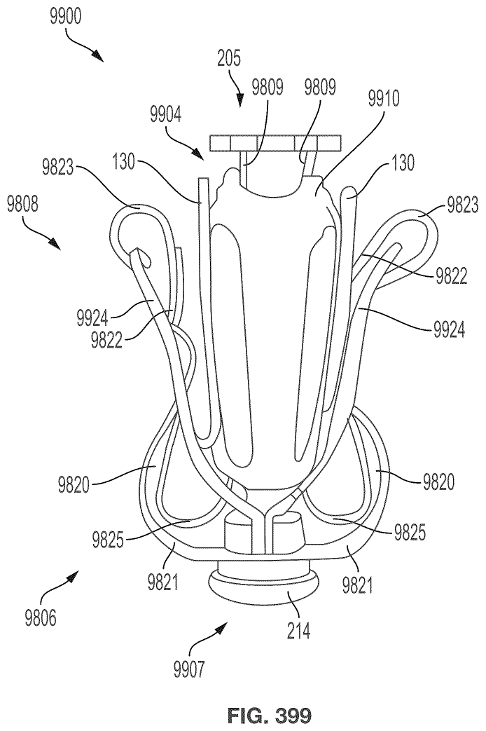

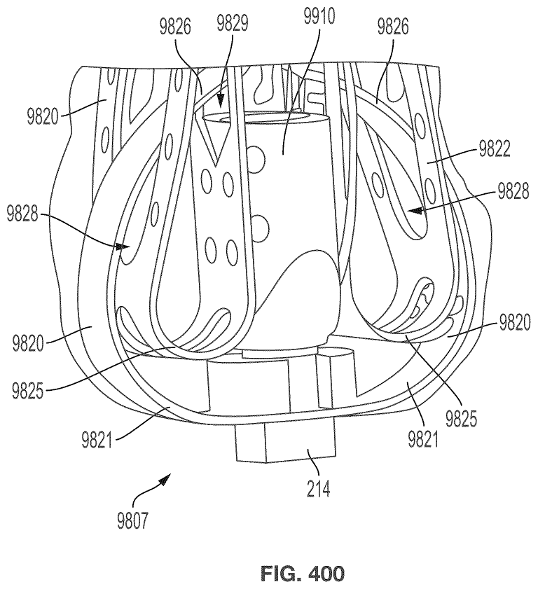

Referring to FIG. 400, a partial perspective view of the distal portion 9807 of the anchor portion 9806 of FIGS. 398-399 attached to the cap 214 and sealing plug that fits within a distal end of the device/implant 9900 (not shown in FIG. 400) is illustrated. The anchor portion 9806 includes an aperture 9848 (FIG. 395) where the outer paddles 9820 are attached at a distal portion 9807.

The aperture 9848 is configured to facilitate attachment of the cap 214 cap to the anchor portion 9806. The aperture 9848 can be configured in any suitable manner. For example, the aperture 9848 can be complementary shaped to a portion of the cap 214. To attach the cap 214 can receive a portion of the cap 214 therethrough.

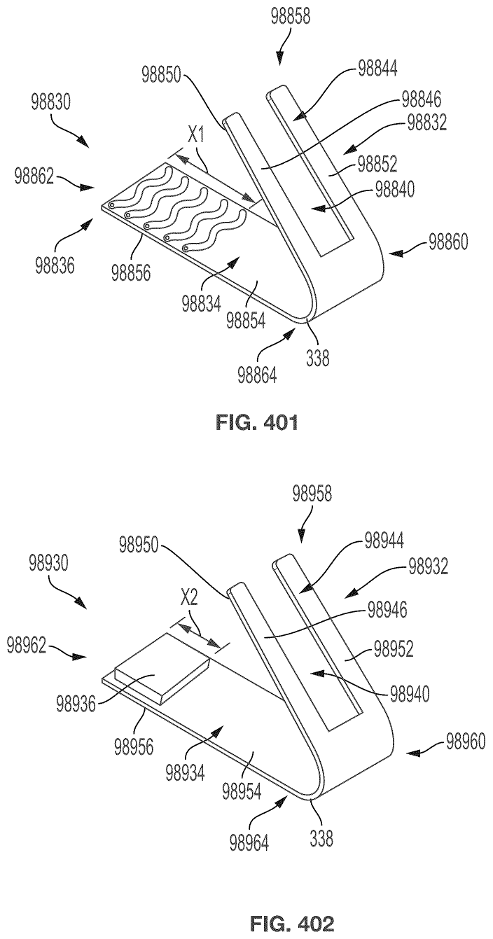

Referring to FIG. 401, a perspective view of an example implementation of a clasp 98830 for a device or implant is shown. The clasp 98830 can be configured and operate similar to the clasp 9630 described above. For example, the clasp 98830 includes a base or fixed arm 98832, a moveable arm 98834, securing elements 98836, and a joint portion 338.

The fixed arm 98832, the moveable arm 98834, and the joint portion 338 can be configured in a variety of ways. In the illustrated example, the fixed arm 98832 includes an inner surface 98850 and an outer surface 98852 parallel and opposite the inner surface 98850. In some implementations, however, the inner surface 98850 is not parallel to the outer surface 98852.

In the illustrated example, the fixed arm 98832 is bifurcated and open-ended. In particular, the fixed arm 98832 includes a first fixed arm portion 98844 and a second fixed arm portion 98846 separated by an open area 98840 such that the fixed arm 98832 resembles a U-shape. In some implementations, the first fixed arm portion 98844 is parallel to the second fixed arm portion 98846. In some implementations, however, the first fixed arm portion 98844 is not parallel to the second fixed arm portion 98846. In some implementations, the fixed arm 98832 can be closed ended while still including the open area 98840 or can be a solid arm without being bifurcated or including an open area.

In the illustrated example, the moveable arm 98834 is generally rectangular with an inner surface 98854 and an outer surface 98856 parallel and opposite the inner surface 98854. In some implementations, however, the inner surface 98854 not parallel to the outer surface 98856. In some implementations, the moveable arm 98834 can be bifurcated and open ended defining an open area or can be closed ended while still including the open area.

The fixed arm 98832 includes a distal end portion 98858 and a proximal portion 98860 opposite the distal end portion 98858. The moveable arm 98834 includes a distal end portion 98862 and a proximal portion 98864 opposite the distal end portion 98862. The proximal portion 98860 of the fixed arm 98832 is joined to the proximal portion 98864 of the moveable arm 98834 by the joint portion 338.

The joint portion 338 connects the fixed arm 98832 to the moveable arm 98834. The joint portion 338 provides a spring force between the fixed and moveable arms 98832, 98834 that biases the clasp to the closed position. The joint portion 338 can be any suitable joint, such as a flexible joint, a spring joint, a pivot joint, or the like. In some implementations, the joint portion 338 is a flexible piece of material integrally formed with the fixed and moveable arms 98832, 98834.

The securing elements 98836 can be configured in a variety of ways. In the illustrated example, the securing elements 98836 are friction-enhancing elements that provide a secure grip on the leaflet of a native valve when the leaflet is between the fixed arm 98832 and the moveable arm 98834 when the clasp 98830 is in a closed position. The friction-enhancing elements can be configured to secure the leaflet in place without piercing the leaflet. The friction-enhancing elements 98836 can be any suitable elements that facilitate or provide for a secure grip on the leaflet. For example, the friction-enhancing elements 98836 can include a rough surface area, such as a knurled, bumpy, or coarse portion. The friction-enhancing elements can include one or more materials that enhance friction. The material can be applied on the clasp 98830 at a variety of locations and in a variety of patterns.

In the illustrated example, the friction-enhancing elements 98836 comprise a rough surface area on the inner surface 98854 of the moveable arm 98834. The area on the inner surface 98854 that includes friction-enhancing elements 98836 extends from the distal end portion 98862 of the moveable arm 98834 toward the proximal portion 98864 a distance X1 which can be 20%, 30%, 40%, 50%, or greater than 50% of a length of the moveable arm 98834.

Referring to FIG. 402, a perspective view of an example implementation of a clasp 98930 for a device or implant is shown. The clasp 98930 can be configured and operate similar to the clasp 9630 described above. For example, the clasp 98930 includes a base or fixed arm 98932, a moveable arm 98934, securing element(s) 98936, and a joint portion 338.

The fixed arm 98932, the moveable arm 98934, and the joint portion 338 can be configured in a variety of ways. In the illustrated example, the fixed arm 98932 includes an inner surface 98950 and an outer surface 98952 parallel and opposite the inner surface 98950. In some implementations, however, the inner surface 98950 is not parallel to the outer surface 98952.

In the illustrated example, the fixed arm 98932 is bifurcated and open-ended. In particular, the fixed arm 98932 includes a first fixed arm portion 98944 and a second fixed arm portion 98946 separated by an open area 98940 such that the fixed arm 98932 resembles a U-shape. In some implementations, the first fixed arm portion 98944 is parallel to the second fixed arm portion 98946. In some implementations, however, the first fixed arm portion 98944 is not parallel to the second fixed arm portion 98946. In some implementations, the fixed arm 98932 can be closed ended while still including the open area 98940 or can be a solid arm without being bifurcated or including an open area.

In the illustrated example, the moveable arm 98934 is generally rectangular with an inner surface 98954 and an outer surface 98956 parallel and opposite the inner surface 98954. In some implementations, however, the inner surface 98954 is not parallel to the outer surface 98956. In some implementations, the moveable arm 98934 can be bifurcated and open ended defining an open area or can be closed ended while still including the open area.

The fixed arm 98932 includes a distal end portion 98958 and a proximal portion 98960 opposite the distal end portion 98958. The moveable arm 98934 includes a distal end portion 98962 and a proximal portion 98964 opposite the distal end portion 98962. The proximal portion 98960 of the fixed arm 98932 is joined to the proximal portion 98964 of the moveable arm 98934 by the joint portion 338.

The joint portion 338 connects the fixed arm 98932 to the moveable arm 98934. The joint portion 338 provides a spring force between the fixed and moveable arms 98932, 98934 that biases them toward one another. The joint portion 338 can be any suitable joint, such as a flexible joint, a spring joint, a pivot joint, or the like. In some implementations, the joint portion 338 is a flexible piece of material integrally formed with the fixed and moveable arms 98932, 98934.

The securing elements 98936 can be configured in a variety of ways. In the illustrated example, the securing elements 98936 are friction-enhancing elements that provide a secure grip on the leaflet of a native valve when the leaflet is between the fixed arm 98932 and the moveable arm 98934 when the clasp 98930 is in a closed position. The friction-enhancing elements can be configured to secure the leaflet in place without piercing the leaflet. The friction-enhancing elements 98936 can be any suitable elements that facilitate or provide for a secure grip on the leaflet. For example, the friction-enhancing elements 98936 can include a rough surface area, such as a knurled, bumpy, or coarse portion. The friction-enhancing elements can include one or more materials that enhance friction. The material can be applied on the clasp 98930 at a variety of locations and in a variety of patterns.

In the illustrated example, the friction-enhancing elements 98936 comprise a friction-enhancing material adhered to the inner surface 98954 of the moveable arm 98934. The friction-enhancing material can be applied to the inner surface 98954 by any suitable manner, such as by a coating or an adhesive suitable for a device or implant. The area on the inner surface 98954 that includes friction-enhancing elements 98936 extends from the distal end portion 98962 of the moveable arm 98934 toward the proximal portion 98964 a distance X2 which can be 30%, 40%, 50%, or greater than 50% of a length of the moveable arm 98934.

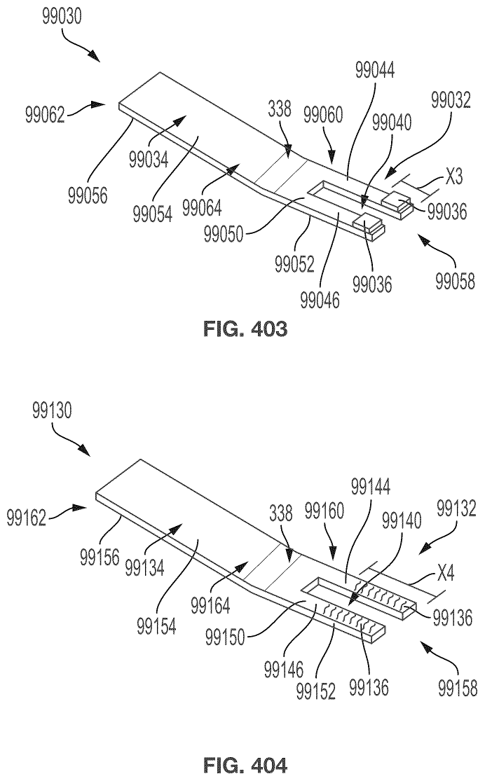

Referring to FIG. 403, a perspective view of an example implementation of a clasp 99030 for a device or implant is shown. The clasp 99030 can be configured and operate similar to the clasp 99030 described above. For example, the clasp 99030 includes a base or fixed arm 99032, a moveable arm 99034, securing elements 99036, and a joint portion 338.

The fixed arm 99032, the moveable arm 99034, and the joint portion 338 can be configured in a variety of ways. In the illustrated example, the fixed arm 99032 includes an inner surface 99050 and an outer surface 99052 parallel and opposite the inner surface 99050. In some implementations, however, the inner surface 99050 is not parallel to the outer surface 99052.

In the illustrated example, the fixed arm 99032 is bifurcated and open-ended. In particular, the fixed arm 99032 includes a first fixed arm portion 99044 and a second fixed arm portion 99046 separated by an open area 99040 such that the fixed arm 99032 resembles a U-shape. In some implementations, the first fixed arm portion 99044 is parallel to the second fixed arm portion 99046. In some implementations, however, the first fixed arm portion 99044 is not parallel to the second fixed arm portion 99046. In some implementations, the fixed arm 99032 can be closed ended while still including the open area 99040 or can be a solid arm without being bifurcated or including an open area.

In the illustrated example, the moveable arm 99034 is generally rectangular with an inner surface 99054 and an outer surface 99056 parallel and opposite the inner surface 99054. In some implementations, however, the inner surface 99054 is not parallel to the outer surface 99056. In some implementations, the moveable arm 99034 can be bifurcated and open ended defining an open area or can be closed ended while still including the open area.

The fixed arm 99032 includes a distal end portion 99058 and a proximal portion 99060 opposite the distal end portion 99058. The moveable arm 99034 includes a distal end portion 99062 and a proximal portion 99064 opposite the distal end portion 99062. The proximal portion 99060 of the fixed arm 99032 is joined to the proximal portion 99064 of the moveable arm 99034 by the joint portion 338.

The joint portion 338 connects the fixed arm 99032 to the moveable arm 99034. The joint portion 338 provides a closing spring force between the fixed and moveable arms 99032, 99034. The joint portion 338 can be any suitable joint, such as a flexible joint, a spring joint, a pivot joint, or the like. In some implementations, the joint portion 338 is a flexible piece of material integrally formed with the fixed and moveable arms 99032, 99034.

The securing elements 99036 can be configured in a variety of ways. In the illustrated example, the securing elements 99036 are friction-enhancing elements that provide a secure grip on the leaflet of a native valve when the leaflet is between the fixed arm 99032 and the moveable arm 99034 when the clasp 99030 is in a closed position. The friction-enhancing elements can be configured to secure the leaflet in place without piercing the leaflet. The friction-enhancing elements 99036 can be any suitable elements that facilitate or provide for a secure grip on the leaflet. For example, the friction-enhancing elements 99036 can include a rough surface area, such as a knurled, bumpy, or coarse portion. The friction-enhancing elements can include one or more materials that enhance friction. The material can be applied on the clasp 99030 in a variety of locations and in a variety of patterns.

In the illustrated example, the friction-enhancing elements 99036 comprise a friction-enhancing material adhered to the inner surface 99050 of the first fixed arm portion 99044 and a second fixed arm portion 99046. The friction-enhancing material can be applied to the inner surface 99050 by any suitable manner, such as a coating or an adhesive suitable for a device or implant. The area on the inner surface 99050 that includes friction-enhancing elements 99036 extends from the distal end portion 99058 of each of the first fixed arm portion 99044 and a second fixed arm portion 99046 toward the proximal portion a distance X3 which can be 20%, 30%, 40%, 50%, or greater than 50% of a length of the fixed arm 99032.

Referring to FIG. 404, a perspective view of an example implementation of a clasp 99130 for a device or implant is shown. The clasp 99130 can be configured and operate similar to the clasp 99130 described above. For example, the clasp 99130 includes a base or fixed arm 99132, a moveable arm 99134, securing elements 99136, and a joint portion 338.

The fixed arm 99132, the moveable arm 99134, and the joint portion 338 can be configured in a variety of ways. In the illustrated example, the fixed arm 99132 includes an inner surface 99150 and an outer surface 99152 parallel and opposite the inner surface 99150. In some implementations, however, the inner surface 99150 is not parallel to the outer surface 99152.

In the illustrated example, the fixed arm 99132 is bifurcated and open-ended. In particular, the fixed arm 99132 includes a first fixed arm portion 99144 and a second fixed arm portion 99146 separated by an open area 99140 such that the fixed arm 99132 resembles a U-shape. In some implementations, the first fixed arm portion 99144 is parallel to the second fixed arm portion 99146. In some implementations, however, the first fixed arm portion 99144 is not parallel to the second fixed arm portion 99146. In some implementations, the fixed arm 99132 can be closed ended while still including the open area 99140 or can be a solid arm without being bifurcated or including an open area.

In the illustrated example, the moveable arm 99134 is generally rectangular with an inner surface 99154 and an outer surface 99156 that is parallel and opposite the inner surface 99154. In some implementations, however, the inner surface 99154 is not parallel to the outer surface 99156. In some implementations, the moveable arm 99134 can be bifurcated and open ended defining an open area or can be closed ended while still including the open area.

The fixed arm 99132 includes a distal end portion 99158 and a proximal portion 99160 opposite the distal end portion 99158. The moveable arm 99134 includes a distal end portion 99162 and a proximal portion 99164 opposite the distal end portion 99162. The proximal portion 99160 of the fixed arm 99132 is joined to the proximal portion 99164 of the moveable arm 99134 by the joint portion 338.

The joint portion 338 connects the fixed arm 99132 to the moveable arm 99134. The joint portion 338 provides a spring force between the fixed and moveable arms 99132, 99134. The joint portion 338 can be any suitable joint, such as a flexible joint, a spring joint, a pivot joint, or the like. In some implementations, the joint portion 338 is a flexible piece of material integrally formed with the fixed and moveable arms 99132, 99134.

The securing elements 99136 can be configured in a variety of ways. In the illustrated example, the securing elements 99136 are friction-enhancing elements that provide a secure grip on the leaflet of a native valve when the leaflet is between the fixed arm 99132 and the moveable arm 99134 when the clasp 99130 is in a closed position. The friction-enhancing elements can be configured to secure the leaflet in place without piercing the leaflet. The friction-enhancing elements 99136 can be any suitable elements that facilitate or provide for a secure grip on the leaflet. For example, the friction-enhancing elements 99136 can include a rough surface area, such as a knurled, bumpy, or coarse portion. The friction-enhancing elements can include one or more materials that enhance friction. The material can be applied on the clasp 99130 in a variety of locations and in a variety of patterns.

In the illustrated example, the friction-enhancing elements 99136 comprise a rough surface area on the inner surface 99150 of the first fixed arm portion 99144 and a second fixed arm portion 99146. The area on the inner surface 99154 that includes friction-enhancing elements 98836 extends from the distal end portion 99158 of the moveable arm 99134 toward the proximal portion 99160 a distance X4 which can be 20%, 30%, 40%, 50%, or greater than 50% of a length of the moveable arm 99134.

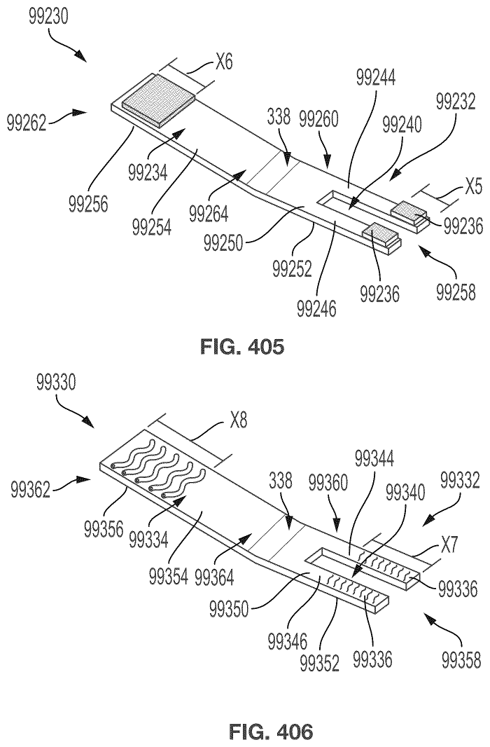

Referring to FIG. 405, a perspective view of an example implementation of a clasp 99230 for a device or implant is shown. The clasp 99230 can be configured and operate similar to the clasp 99230 described above. For example, the clasp 99230 includes a base or fixed arm 99232, a moveable arm 99234, securing elements 99236, and a joint portion 338.

The fixed arm 99232, the moveable arm 99234, and the joint portion 338 can be configured in a variety of ways. In the illustrated example, the fixed arm 99232 includes an inner surface 99250 and an outer surface 99252 parallel and opposite the inner surface 99250. In some implementations, however, the inner surface 99250 is not parallel to the outer surface 99252.

In the illustrated example, the fixed arm 99232 is bifurcated and open-ended. In particular, the fixed arm 99232 includes a first fixed arm portion 99244 and a second fixed arm portion 99246 separated by an open area 99240 such that the fixed arm 99232 resembles a U-shape. In some implementations, the first fixed arm portion 99244 is parallel to the second fixed arm portion 99246. In some implementations, however, the first fixed arm portion 99244 is not parallel to the second fixed arm portion 99246. In some implementations, the fixed arm 99232 can be closed ended while still including the open area 99240 or can be a solid arm without being bifurcated or including an open area.

In the illustrated example, the moveable arm 99234 is generally rectangular with an inner surface 99254 and an outer surface 99256 parallel and opposite the inner surface 99254. In some implementations, however, the inner surface 99254 is not parallel to the outer surface 99256. In some implementations, the moveable arm 99234 can be bifurcated and open ended defining an open area or can be closed ended while still including the open area.

The fixed arm 99232 includes a distal end portion 99258 and a proximal portion 99260 opposite the distal end portion 99258. The moveable arm 99234 includes a distal end portion 99262 and a proximal portion 99264 opposite the distal end portion 99262. The proximal portion 99260 of the fixed arm 99232 is joined to the proximal portion 99264 of the moveable arm 99234 by the joint portion 338.

The joint portion 338 connects the fixed arm 99232 to the moveable arm 99234. The joint portion 338 provides a closing spring force between the fixed and moveable arms 99232, 99234. The joint portion 338 can be any suitable joint, such as a flexible joint, a spring joint, a pivot joint, or the like. In some implementations, the joint portion 338 is a flexible piece of material integrally formed with the fixed and moveable arms 99232, 99234.

The securing elements 99236 can be configured in a variety of ways. In the illustrated example, the securing elements 99236 are friction-enhancing elements that provide a secure grip on the leaflet of a native valve when the leaflet is between the fixed arm 99232 and the moveable arm 99234 when the clasp 99230 is in a closed position. The friction-enhancing elements can be configured to secure the leaflet in place without piercing the leaflet. The friction-enhancing elements 99236 can be any suitable elements that facilitate or provide for a secure grip on the leaflet. For example, the friction-enhancing elements 99236 can include a rough surface area, such as a knurled, bumpy, or coarse portion. The friction-enhancing elements can include one or more materials that enhance friction. The material can be applied on the clasp 99230 in a variety of locations and in a variety of patterns.

In the illustrated example, the friction-enhancing elements 99236 comprise a friction-enhancing material adhered to the inner surface 99250 of the first fixed arm portion 99244 and a second fixed arm portion 99246 and on the inner surface 99254 of the moveable arm portion 99234. The friction-enhancing material can be applied to the inner surfaces 99250, 99254 by any suitable manner, such as a coating or an adhesive suitable for a device or implant. The area on the inner surface 99250 of the fixed arm portion 99232 that includes friction-enhancing elements 99236 extends from the distal end portion 99258 of each of the first fixed arm portion 99244 and a second fixed arm portion 99246 toward the proximal portion 99260 a distance X5 which can be 20%, 30%, 40%, 50%, or greater than 50% of a length of the fixed arm 99232. The area on the inner surface 99254 of the moveable arm 99234 that includes friction-enhancing elements 99236 extends from the distal end portion 99262 of the moveable arm 99234 toward the proximal portion 99264 a distance X6 which can be 20%, 30%, 40%, 50%, or greater than 50% of a length of the moveable arm 99234.

Referring to FIG. 406, a perspective view of an example implementation of a clasp 99330 for a device or implant is shown. The clasp 99330 can be configured and operate similar to the clasp 99330 described above. For example, the clasp 99330 includes a base or fixed arm 99332, a moveable arm 99334, securing elements 99336, and a joint portion 338.

The fixed arm 99332, the moveable arm 99334, and the joint portion 338 can be configured in a variety of ways. In the illustrated example, the fixed arm 99332 includes an inner surface 99350 and an outer surface 99352 parallel and opposite the inner surface 99350. In some implementations, however, the inner surface 99350 is not parallel to the outer surface 99352.

In the illustrated example, the fixed arm 99332 is bifurcated and open-ended. In particular, the fixed arm 99332 includes a first fixed arm portion 99344 and a second fixed arm portion 99346 separated by an open area 99340 such that the fixed arm 99332 resembles a U-shape. In some implementations, the first fixed arm portion 99344 is parallel to the second fixed arm portion 99346. In some implementations, however, the first fixed arm portion 99344 is not parallel to the second fixed arm portion 99346. In some implementations, the fixed arm 99332 can be closed ended while still including the open area 99340 or can be a solid arm without being bifurcated or including an open area.

In the illustrated example, the moveable arm 99334 is generally rectangular with an inner surface 99354 and an outer surface 99356 parallel and opposite the inner surface 99354. In some implementations, however, the inner surface 99354 is not parallel to the outer surface 99356. In some implementations, the moveable arm 99334 can be bifurcated and open ended defining an open area or can be closed ended while still including the open area.

The fixed arm 99332 includes a distal end portion 99358 and a proximal portion 99360 opposite the distal end portion 99358. The moveable arm 99334 includes a distal end portion 99362 and a proximal portion 99364 opposite the distal end portion 99362. The proximal portion 99360 of the fixed arm 99332 is joined to the proximal portion 99364 of the moveable arm 99334 by the joint portion 338.

The joint portion 338 connects the fixed arm 99332 to the moveable arm 99334. The joint portion 338 provides a closing spring force between the fixed and moveable arms 99332, 99334. The joint portion 338 can be any suitable joint, such as a flexible joint, a spring joint, a pivot joint, or the like. In some implementations, the joint portion 338 is a flexible piece of material integrally formed with the fixed and moveable arms 99332, 99334.

The securing elements 99336 can be configured in a variety of ways. In the illustrated example, the securing elements 99336 are friction-enhancing elements that provide a secure grip on the leaflet of a native valve when the leaflet is between the fixed arm 99332 and the moveable arm 99334 when the clasp 99330 is in a closed position. The friction-enhancing elements can be configured to secure the leaflet in place without piercing the leaflet. The friction-enhancing elements 99336 can be any suitable elements that facilitate or provide for a secure grip on the leaflet. For example, the friction-enhancing elements 99336 can include a rough surface area, such as a knurled, bumpy, or coarse portion. The friction-enhancing elements can include one or more materials that enhance friction. The material can be applied on the clasp 99330 in a variety of locations and in a variety of patterns.

In the illustrated example, the friction-enhancing elements 99336 comprise a rough surface area on the inner surface 99350 of the first fixed arm portion 99344 and a second fixed arm portion 99346 and on the inner surface 99354 of the moveable arm portion 99334. The area on the inner surface 99350 of the fixed arm portion 99332 that includes friction-enhancing elements 99336 extends from the distal end portion 99358 of each of the first fixed arm portion 99344 and a second fixed arm portion 99346 toward the proximal portion 99360 a distance X7 which can be 20%, 30%, 40%, 50%, or greater than 50% of a length of the fixed arm 99332. The area on the inner surface 99354 of the moveable arm 99334 that includes friction-enhancing elements 99336 extends from the distal end portion 99362 of the moveable arm 99334 toward the proximal portion 99364 a distance X8 which can be 20%, 30%, 40%, 50%, or greater than 50% of a length of the moveable arm 99334.

It is often desirable to change the paddle width in the devices (e.g., implantable device/implant 200) while maneuvering, seating, and deploying the device. Many the device designs use a tensile activation system for this purpose. For example, tension can be applied to one or more lines or sutures to narrow portions of a paddle. During such procedures the paddle frames 224 can be stopped, locked and held in wide, narrow, and/or intermediate positions. Locking can also be helpful prior to leaflet capture to keep the paddle frames 224 narrow enough to avoid potential obstructions, such as interactions with the chordae tendineae (CT, FIGS. 3 and 5). Therefore, it would be advantageous to be able to lock a tensile system in place so that a paddle width could be maintained without active tensioning by the user. Such a locking system would free the user to concentrate on tasks other than maintaining the paddle width, e.g., maneuvering the device into place and opening and closing the device to capture the leaflets of the native heart valve.

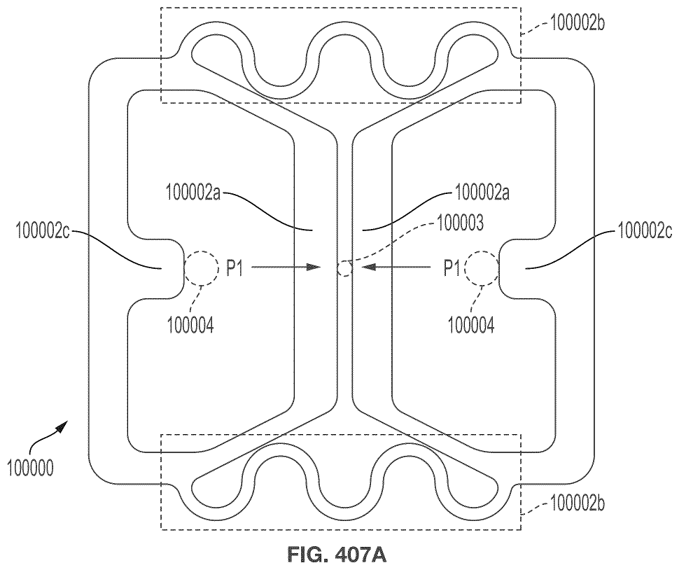

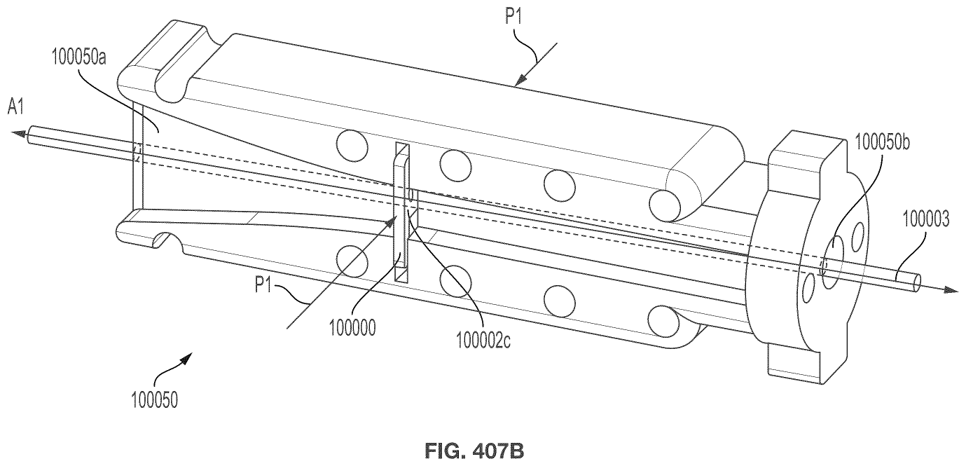

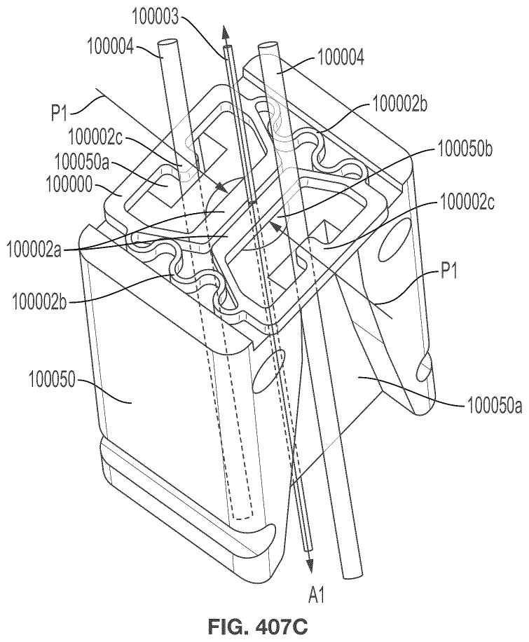

A wide variety of different locking devices can be used to lock the positions width adjustment elements (e.g., width adjustment wire, width adjustment shaft, width adjustment tube, width adjustment line, width adjustment cord, width adjustment suture, etc.), such as the illustrated one or more control lines 100003 or sutures. FIG. 407A illustrates an example holding or locking mechanism 100000 that can hold or lock an actuation line(s) 100003, such as a suture, wire or shaft at a user-defined position. FIG. 407B shows the example holding or locking mechanism 100000 positioned in a receiver, such as the illustrated housing 100050, such as a housing of a valve repair device, such as any of the valve repair devices disclosed herein. FIG. 407C is a cut-away view of the mechanism 100000 positioned in the housing 100050. In FIG. 407A, the line(s) 100003 is represented in cross-section. One line is shown. However, the mechanism can be used to lock the positions of any number of lines. For example, one, two, three, four, etc. lines 100003 can be positioned in the area where one line(s) 100003 is illustrated in FIG. 407A. In FIGS. 407B and 407C line(s) 100003 is shown in profile. In both FIGS. 407B and 407C, line(s) 100003 is partially transparent to more clearly illustrate its interactions with the mechanism 100000. The line(s) 100003 adjust the width of the paddle frames 224 by moving in and out of the page in FIG. 407A and along axis A1 in FIGS. 407B and 407C.

The housing 100050 can include a slot 100050a, shown in FIG. 407B. The line(s) 100003 can traverse the housing 100050 via a hole 100050b. In FIG. 407B, the hole 100050b is shown at the distal end of the housing where line(s) 100003 exits to connect to the paddle frames 224. It is to be understood that this arrangement is merely example and that other suitable arrangements are contemplated within the context of this disclosure. For example, the preceding descriptions of the ends of the housing 100050 with respect to the user and the paddle frames 224 can be reversed. Moreover, although the mechanism 100000 is shown in FIGS. 407B and 407C as deployed in the housing 100050, there are other ways of deploying the mechanism 100000 with or without the housing 100050. Any manner of deploying the mechanism 100000 are within the context of this disclosure.

The mechanism 100000 functions as a brake on the motion of line(s) 100003 along axis A1 by applying a friction load to the outer walls of line(s) 100003. The friction load is applied via friction bearing members 100002a (e.g., clamp surfaces, clamp bars, etc.) Specifically, the friction bearing members 100002a undergo a pinching motion P1 to provide friction to line(s) 100003. The pinching motion Pa is actuated by the resilient members 100002b (e.g., springs, etc.) on either side of the mechanism 100000. The resilient members 100002b are biased such that they provide a resilient force that resists motion of the friction bearing members 100002a in the opposite direction of P1. This creates a default configuration for the mechanism 100000 in which it applies a friction load on line(s) 100003 unless another force opposes the resilient force. The friction load applied by the friction bearing members 100002a can lock the line(s) 100003 in place or otherwise retard its motion along axis A1.

The mechanism 100000 can be used in a wide variety of different ways. For example, the mechanism 100000 can be used to lock the position of the lines used to widen and narrow the paddles of any of the devices shown in FIGS. 127-136, 157-158, and 164-168 and/or to lock the devices shown in FIGS. 30-33 in an open position, a closed position, or any position in between. In FIGS. 127-136, 157-158, and 164-168 the tension is applied to the lines to narrow the paddles and tension is released to widen the paddles. The mechanism 100000 can be used to set the position(s) of the lines that are used to control the width of the paddle frames.

In some implementations, the actuation element 212 of the device illustrated by FIGS. 30-33 can be stopped/secured by the mechanism shown in FIGS. 407A-407C instead of the illustrated one or more lines 100003. As discussed above in the context of FIGS. 30-33, the actuation element 212 is in mechanical communication with the outer paddles 220 and the paddle frames 224. Extending the actuation element 212 pulls down on the outer paddles 220 and the paddle frames 224 and causes them to flex from the closed position shown in FIG. 23 to the partially open position shown in FIGS. 30-31 or the extended open positions shown in FIGS. 32-36. If the outer paddles 220 and the paddle frames 224 are in the open positions shown in FIGS. 30-36, retracting the actuation element 212 pulls up on the outer paddles 220 and the paddle frames 224, causing them to move back to the closed position shown in FIG. 23. The mechanism 100000 can be employed to lock the paddle frames 224 in any of these positions, as well as any position intermediate between them. In some implementations, the mechanism 100000 is used to lock both lines or sutures that control the width of the paddles and the wire or shaft that controls how open or closed the paddles of the device are.

Referring again to FIGS. 407A-407C, regardless of the use of the mechanism 100000, an opposing force to the resilient force provided by the resilient members 100002b can release line(s) 100003 from a locked position. The opposing force can be applied by the control members 100004 (e.g., lines, rods, tubes, bars, etc.) shown in cross-section in FIG. 407A and in profile in FIG. 407C. In FIG. 407C, the control members 100004 are partially transparent to more clearly illustrate its interactions with the mechanism 100000 and the housing 100050. Although the control members 100004 are not shown in FIG. 407B, they would appear beside line(s) 100003 so that they can bear on the control nodules 100002c. The control members 100004 can be rods or cylindrical structures actuated by the user. Such rods are shown in FIG. 407C. The control members 100004 can be accommodated by slots 100050a (FIGS. 407B and 407C.

The control members 100004 can oppose the resilient force provided by the resilient members 100002b by pressing on the control nodules 100002c in a direction opposite to P1. The user can actuate this opposing force by manually moving or flexing the control members 100004. For example, when the control members 100004 are tapered, advancing (or retracting, depending on the direction of taper) will move the control nodules apart. Doing so with sufficient force to overcome the resilient force provided by the resilient members 100002b will prevent or inhibit the friction bearing members 100002a from pinching the outer walls of line(s) 100003. This will allow line(s) 100003 to move in the direction in and out of the page in FIG. 407A and along the axis A1 in FIGS. 407B and 407C.

There are other ways to actuate the opposing force depending on the construction of the control members 100004 and/or the housing 100050. For example, as mentioned above the control member rods 100004 can be constructed with a tapered or sloping outer surface (e.g., the control members 100004 can be cylindrical, with a glancing slice taken out of their volume at the side (not shown)). The resulting, sloping outer surface would then bear on the control nodules 100002c. In this way, the position of the control members 100004 along axis A1 could control the amount of force applied on the control nodules 100002c by effectively controlling the cross-section of the control members 100004 (see, e.g., FIG. 407A) presented to the mechanism 100000. This is because displacing the control members 100004 along axis A1 would control which portion of the sloping surface is in contact with the control nodules 100002c, thus changing the force applied in the opposite direction of P1. In this configuration, the user could simply push or pull on the control members 100004 along axis A1 to control braking of, and therefore motion of, line(s) 100003. Still another way of activating the control nodules 100002c would be to provide a hinged wall to the housing 100050 (not shown) that could bear on the control nodules 100002c in a similar manner described above with respect to the control members 100004. Still other ways of controlling the friction on line(s) 100003 are also within the scope of the present disclosure.

The mechanism 100000 can be fashioned from a single piece of material (e.g., laser cut from a metal such as a shape memory alloy, e.g., nitinol, or aluminum). Alternatively, the mechanism 100000 can comprise sections or portions that are fashioned separately. For example, the resilient members 100002b can be fabricated separately and added to the mechanism 100000.

Although FIGS. 407A and 407C show the mechanism 100000 including the resilient members 100002b as springs, it is to be understood that other configurations are possible. For example, the resilient members 100002b can include a coil, spring, elastic member, and or other energy storage device.

Other configurations of the resilient members 100002b can include gears or active components configured to retard, resist, or generate motion. In some of these cases, the control members 100004 are not needed to unlock line(s) 100003. Simply reversing the direction of the gear or active component can be sufficient. In one such configuration, the resilient members 100002b can include a linear worm-gear drive (not shown) that interfaces with the friction bearing members 100002a. The worm gear could be operated in forward and reverse directions to push the friction bearing members 100002a along P1 or withdraw them, respectively. Similarly, the resilient members 100002b can include rachet mechanisms (not shown) with mechanical teeth to interlock the friction bearing members 100002a. Such mechanisms could also be operated in forward and reverse to open and close the friction bearing members 100002a.

As discussed above, it is often necessary to change the paddle width in the devices (e.g., device/implant 200) while maneuvering, seating, and deploying the device. In some implementations, the width of the paddle frames 224 is reduced by pulling a portion of the paddle frames into a cap of the device. The portion of the paddle frames 224 that are pulled into the cap can be sharply bent, introducing a large amount of stress into the bent portion of the paddle frame.

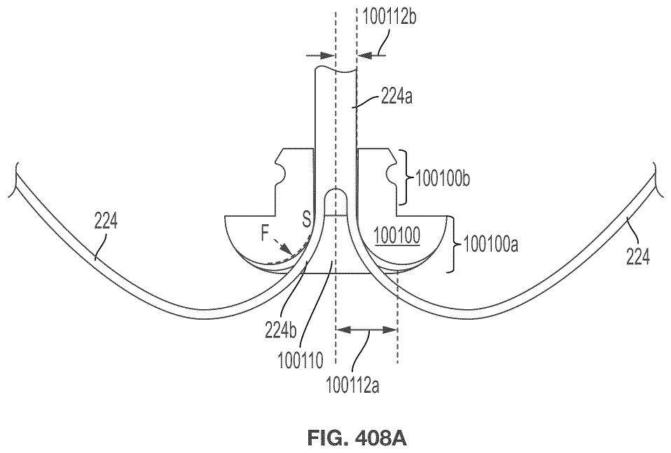

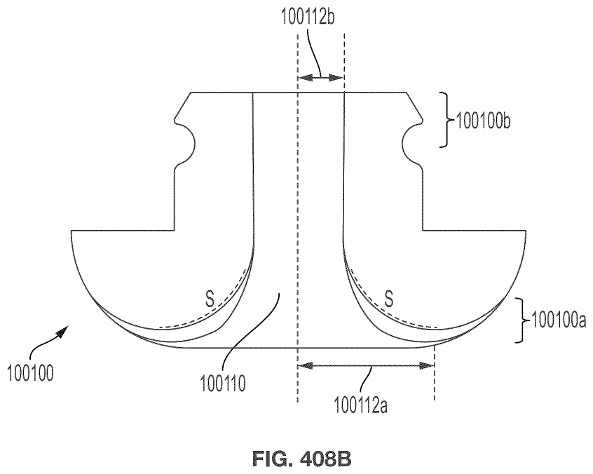

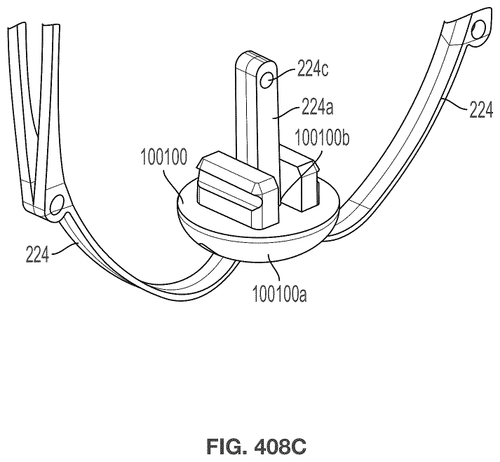

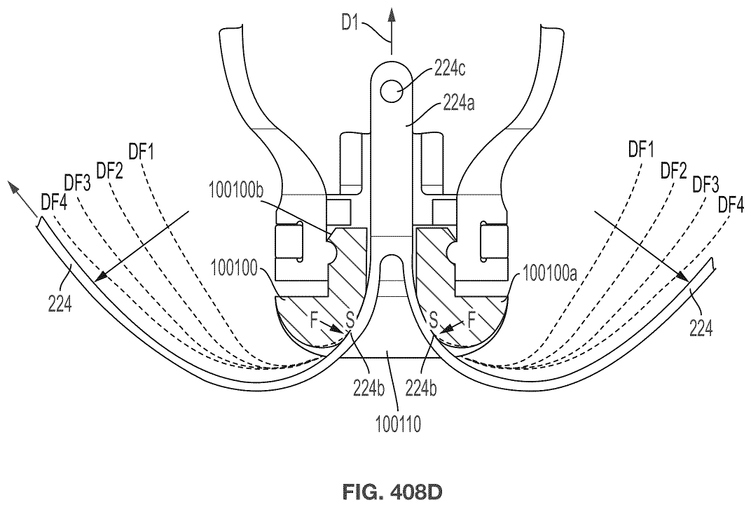

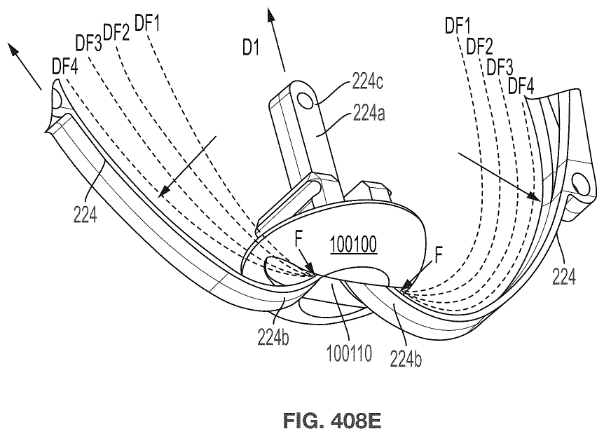

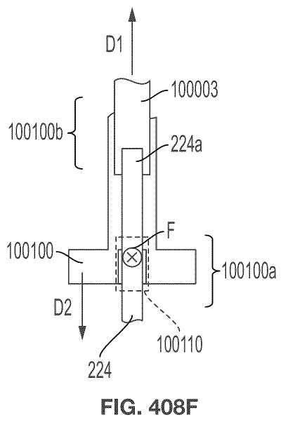

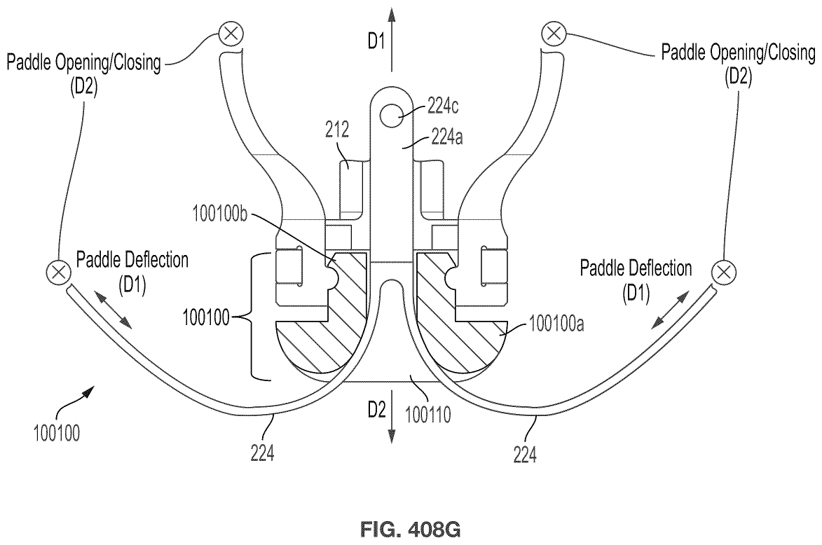

FIGS. 408A-408G illustrate an example the cap 100100 that reduces stress applied to portions of a paddle frame 224 that are pulled through the cap by providing a radiused entry point or hole into the cap 100100. This radiused entry point increases the radius of curvature of the portion of the paddle frame that is pulled into the cap and therefore reduces the stress that is introduced into the paddle frame. The radiused hole or entry point 100110 in the cap 100100 can guide the paddle frames 224 through a series of deflections.

FIG. 408A shows the cap 100100 engaged with the paddle frame 224. FIG. 408B shows a close-up of the cap 100100 without the paddle frame 224 for clarity. Both FIGS. 408A and 408B show the cap 100100 in cross-section. FIG. 408C shows an external side view of the cap 100100 and the paddle frame 224 arrangement shown in FIG. 408A. As shown in FIGS. 408A and 408B, the cap 100100 includes a radiused hole or entry point 100110 that accommodates an inner end or portion 224a of the paddle frame 224.

The radiused hole or entry point 100110 provides a mechanism via which the cap 100100 can control the paddle frame 224 deflection in a manner that introduces less stress to the paddle frame 224. In particular, the radiused hole 100110 has a radius 100112a at the distal end 100100a of the cap 100100 that is larger than radius 100112b at the proximal end 100100b (FIGS. 408A and 408B). (Note-“Proximal” and “distal” are herein used to refer to relative distances with respect to the user.) The difference in radius between the radius 100112b and the radius 100112a is bridged by a slope S of the portion of the hole 100110 in contact with the paddle frame portion 224b. Because of slope S, relative motion of the cap 100100 with respect to the paddle frame 224 can impart force F (see, e.g., FIG. 408A) on portion 224b. This force F is significantly less than would be the case if the hole 100110 were cylindrical and an inside surface of the hole and a distal end of the cap form a right angle. Since the paddle frame 224 is generally made of material that can substantially hold its shape without plastically deforming, force F tends to deflect the paddle frame 224 upward/downward throughout its length.

The paddle frame 224 can include an attachment portion 224c that allows the paddle frame 224 to attach directly to another portion of the device for mechanical communication. For example, attachment portion 224c can be attached to a mechanism for pulling of the paddle frames 224 into the cap to reduce the width of the paddle frames and pushing the paddle frames 224 out of the cap to increase the width of the paddle frames.

Deflection of the paddle frame 224 via cap 100100 is shown in more detail in FIGS. 408D and 408E. FIG. 408D shows the motion in cross-section, while FIG. 408E shows the same motion from a perspective exterior, side view. FIGS. 408D and 408E show a range of deflection DF1-DF4 facilitated by moving the paddle frame 224 along direction D1 into the cap 100100 (e.g., as the paddle frames 224 are pulled into the cap). Direction D1 extends from the distal end 100100a to the proximal end 100100b of the cap 100100. The hole 100110 extends from the proximal end 100100b of the cap 100100 to the distal end 100100a in order to accommodate the inner end or portion 224a of the paddle frame 224. In this way, moving the paddle frame 224 into the cap 100100 in direction D1 causes the paddle frame 224 to deflect toward the cap 100100 (e.g., deflecting the paddle frame 224 from DF4 to DF1). Pushing the paddle frame 224 out of the cap 100100 opposite the direction D1 causes the paddle frame 224 to deflect in the other outward (e.g., deflecting the paddle frame 224 from DF1 to DF4).

FIG. 408F shows how the cap 100100 can be used to both (simultaneously or separately) deflect (expand and contract) the paddle frames 224 (as shown in FIGS. 408D and 408E) and open and close the paddle frames 224 via actuation element 212 (as shown in FIGS. 30-36). FIG. 408F is a cross section of the cap 100100 that is perpendicular to the cross-sectional views shown in FIGS. 408A and 408D. The difference in view is revealed by comparing the relative orientation of force F imparted by the cap 100100 to deflect the paddle frames 224 in FIG. 408F with the orientation of force Fin FIGS. 408A and 408D. In FIG. 408F, force F points into the page. In contrast, force F is parallel to the page in FIGS. 408A and 408D.

In FIG. 408F, motion along D1 represents the same motion of the paddle frames 224 into the cap 100100 discussed in the context of FIGS. 408D and 408E above. On the other hand, motion along D2 represents an actuation or relative motion of actuation element 212 and accompanying movement of the cap 100100 to open and close the paddle frames 224. FIG. 408F shows how motion of the paddle frames into the cap in the direction D1 can occur independently of moving the cap 100100 in direction D1, and vice versa. Motion along of the paddle frames 224 into the cap in direction D1 and movement of the cap in direction D2 can also occur concurrently. That is, motion of actuation element 212 in the direction D2 can open the paddle frames 224 (as shown in FIGS. 30-36) at the same time that motion of the paddle frames into the cap 100100 in direction D1 to deflect the paddles inward (as shown in FIGS. 408D and 408E). FIG. 408G shows the difference in the paddle motion between 1) the deflection caused by pulling the paddle into the cap 100100 in direction D1 and 2) the opening/closing of both the rigid and flexible portions of the paddle frames (into and out of the page) caused by moving the cap with the actuation element 212 in the direction D2.

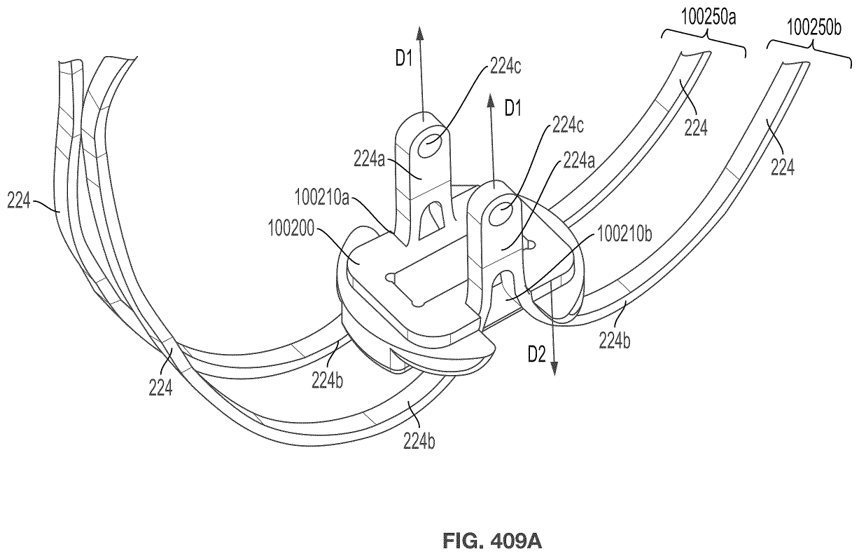

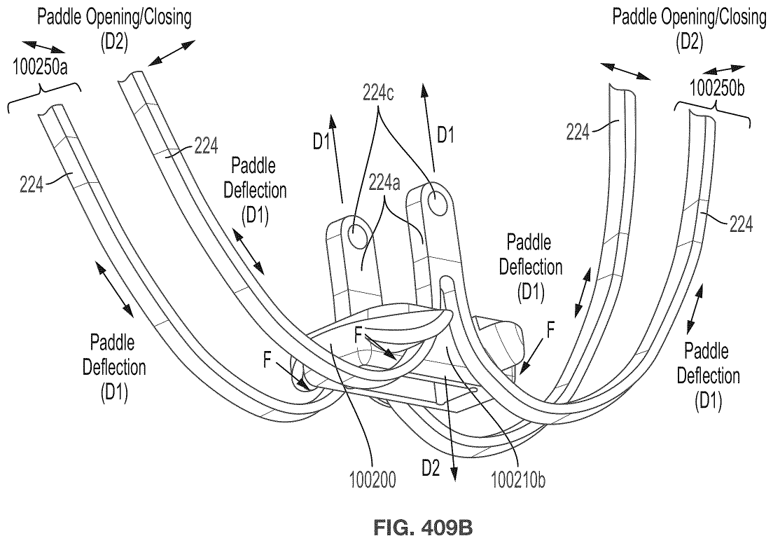

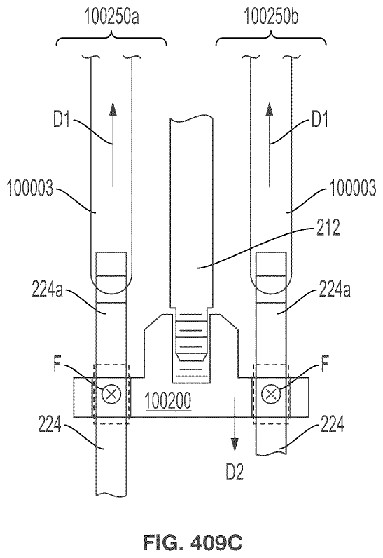

FIGS. 409A-409C show an alternative variation of a cap 100200 that spaces two paddle frames 224 apart in the cap 100200. Many of the devices disclosed herein include two spaced apart paddle frames where the distal ends of the paddle frames are adjacent to one another in the cap (see FIG. 23). In the example of FIGS. 409A-409C, the cap 100200 spaces the distal ends of the paddle frames 224 apart, allows portions of the paddle frames to be pulled into the cap, and is shaped to reduce stress on the paddle frames 224 as the paddle frames are drawn into the cap.

FIG. 409A shows the cap 100200 accommodating two paddle arrangements 100250a and 100250b. The paddle arrangements 100250a and 100250b comprise flexible portions of the paddle frames 224 themselves, along with portions 224a, 224b and 224c discussed in more detail above. Unlike the cap 100100, the cap 100200 has two strain relieving holes or openings 100210a and 100210b. Both of the paddle sets 100250a and 100250b are mated with the cap 100200 such that respective inner ends or portions 224a extend out of the holes 100210a and 100210b, respectively. Both the holes 100210a and 100210b can be substantially similar in construction as hole 100110 shown in FIG. 408B. In particular, the holes 100210a and 100210b can have a larger radius in the distal portion than in the proximal portion and a slope S in between (e.g., as shown for the cap 100100 in FIG. 408B). That is, the holes 100210a and 100210b can be constructed to reduce the deflecting force F (e.g., as shown for the cap 100100 in FIG. 408B) on the paddle sets 100250a and 100250b. The slope S causes the paddle sets 100250a and 100250b to undergo a similar deflecting motion DF1-DF4 as shown in FIGS. 408D and 408E when the paddle frames 224 are pulled into the cap 100200 in the direction D1.

FIG. 409B is a lower side view of the cap 100200 and the paddle sets 100250a and 100250b showing the reduction of force F and resulting stress in the paddle frames due to the radiused openings in the cap 100200. FIG. 409B also shows directions of the paddle deflection when the paddle frames 224 are pulled into the cap 100200 in the direction D1. FIG. 409B also roughly shows the direction of paddle opening and closing when the cap 100200 is moved in the direction D2. These are the same or similar motions to those shown for the paddle frames 224 in response to similar motions of the cap 100100 and the paddle frame inner ends or portions 224a, as shown in FIG. 408G.

FIG. 409C shows a cross section of the cap 100200 that has the same orientation as the cross section for the cap 100100 shown in FIG. 408F. As in FIG. 408F, force F points into the page. In FIG. 409C, motion of the paddle frames into the cap 100200 in the direction D1 represents the same motion of the paddle frames 224 discussed in the context of FIGS. 409A and 409B above, resulting in the paddle narrowing and widening. Motion of the actuation element 212 and attached cap 100200 in the direction D2 (and accompanying the paddle frame inner end or portion 224a) results in opening of the paddle frames 224. As shown in FIG. 409C, the paddle set 100250a and 100250b widening and narrowing can be independent of the opening and closing caused by the movement of the cap 100200 in the direction D2. FIG. 409C also shows how the paddle set 100250a and 100250b can be independent in the direction D1 and independent of the motion of the cap 100200. Therefore, narrowing and widening of the paddle sets 100250a and 100250b, as shown in FIG. 409B, can be accomplished independently of one another. The narrowing and widening of the paddle sets 100250a and 100250b, as shown in FIG. 409B, can also be accomplished simultaneously by pulling simultaneously pulling both of the paddle frames 224 into the cap in the direction D1.

Referring to FIG. 409B, narrowing and widening of the paddles 224 can also be accomplished simultaneously with the opening and closing of the paddle frames. In other words, motion of actuation element 212 in the direction D2 can be used to open and close the paddle frames 224 as shown in FIGS. 30-36 at the same time that motion of lines in the direction D1 can be used to deflect (narrow) the paddles as shown in FIG. 409B. Narrowing and widening of the paddles 224 can also be accomplished independently with the opening and closing of the paddle frames. In other words, motion of actuation element 212 in the direction D2 can be used to open and close the paddle frames 224 as shown in FIGS. 30-36 at a different time than motion of lines in the direction D1 to deflect (narrow) the paddles.

FIG. 409A shows the cap 100200 accommodating two independently narrowed/widened the paddle sets 100250a and 100250b that are spaced apart. The paddle sets 100250a and 100250b comprise the paddle frames 224 themselves, along with inner ends or portions 224a, 224b and 224c discussed in more detail above. Unlike the cap 100100, the cap 100200 has two the holes 100210a and 100210b. Both the paddle sets 100250a and 100250b are mated with the cap 100200 such that respective inner ends or portions 224a extend out of the holes 100210a and 100210b. Both the holes 100210a and 100210b can be substantially similar in construction as hole 100110 shown in FIG. 408B. In particular, the holes 100210a and 100210b can have a larger radius in the distal portion than in the proximal portion and a slope S in between (e.g., as shown for the cap 100100 in FIG. 408B). That is, the holes 100210a and 100210b can be constructed to reduce the deflecting force F (e.g., as shown for the cap 100100 in FIG. 408B) and resulting strain on the paddle sets 100250a and 100250b. This force F causes the paddle sets 100250a and 100250b to undergo a similar deflecting motion DF1-DF4 as shown in FIGS. 408D and 408E when the paddle frames 224 are pulled into the cap 100200 in the direction D1.

FIG. 409B is a lower side view of the cap 100200 and the paddle sets 100250a and 100250b showing the imposition of force F. FIG. 409B also shows directions of the paddle deflection (narrowing/widening) when the paddle frames 224 are pulled into the cap 100200 in the direction D1. FIG. 409B also shows the direction of the paddle opening and closing when the cap pulls the paddle assemblies in the direction D2. These are similar motions to those shown for the paddle frames 224 in response to similar motions of the cap 100100 and the paddle frame inner ends or portions 224a, as shown in FIG. 408G.

FIG. 409C shows a cross section of the cap 100200 that has the same orientation as the cross section for the cap 100100 shown in FIG. 408F. As in FIG. 408F, Force F points into the page. In FIG. 409C, motion of the paddle frames 224 into the cap in the direction D1 represents the same motion of the paddle frame into the cap 100200 discussed in the context of FIGS. 409A and 409B above resulting in the paddle deflection (narrowing). Motion of the actuation element 212 and attached cap (and accompanying the paddle frame inner end or portion 224a) results in opening the paddle frames 224. As shown in FIG. 409C, the paddle set 100250a and 100250b narrowing and widening can occur independently via the two lines being moveable in the direction D1. Therefore, widening and narrowing of the paddle sets 100250a and 100250b, as shown in FIG. 409B, can be accomplished independently of one another.

FIG. 409C also shows how the motion of each of the paddle set 100250a and 100250b in the direction D1 can be independent of the motion of the cap 1001200 to open and close the paddles. Widening and narrowing can also be accomplished simultaneously with opening and closing. In other words, motion of actuation element 212 in the direction D2 can be used to open the paddle frames 224 as shown in FIGS. 30-36 at the same time that pulling the paddle frames 224 into the cap 100200 in the direction D1 can be used to narrow the paddles as shown in FIG. 409B.

As discussed above, in some implementations, the paddle frames 224 can be narrowed/widened, stopped, locked and held in fully expanded, fully narrowed, and in intermediate positions. Locking can also be particularly helpful prior to leaflet the capture in keeping the paddles 224 narrow to traverse potential obstructions, such as the chordae tendineae (CT, FIGS. 3 and 5).

In some implementations, the paddle narrowing and widening adjustment mechanism automatically holds the paddle frames 224 in the adjusted position when the mechanism is released. A wide variety of different mechanisms can be used to narrow and widen the paddle frames and automatically hold the paddle frames 224 in any adjusted position. For example, a screw mechanism, a ratchet mechanism, a cam mechanism, etc. can be used. Such mechanisms allow the user to set and maintain the paddle frames 224 at any width so that a particular the paddle width is maintained without active tensioning action by the user. Additionally, such mechanisms can facilitate more precise control of narrowing/widening.

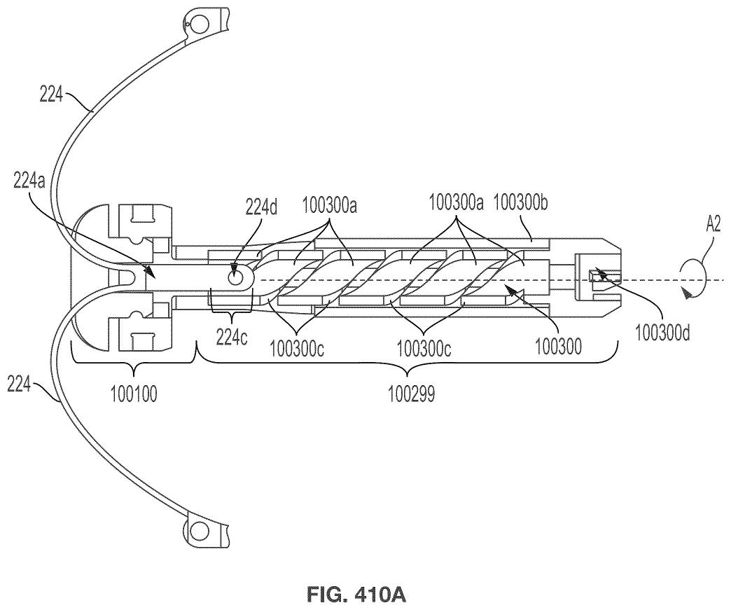

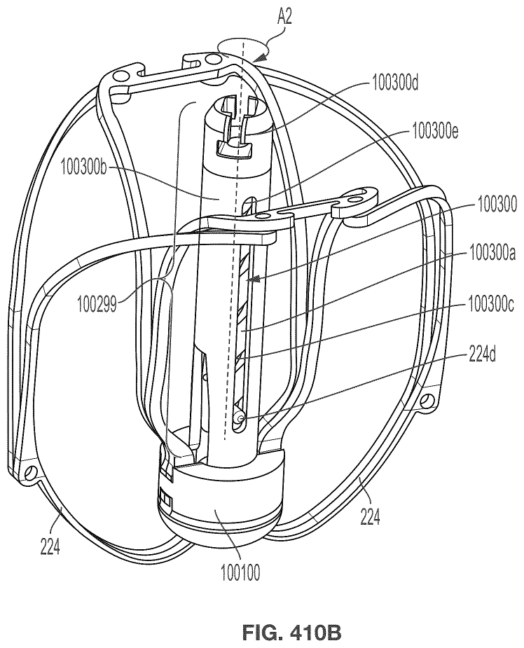

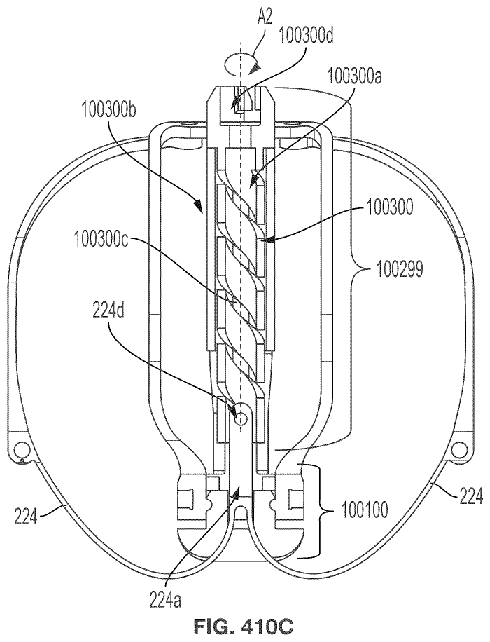

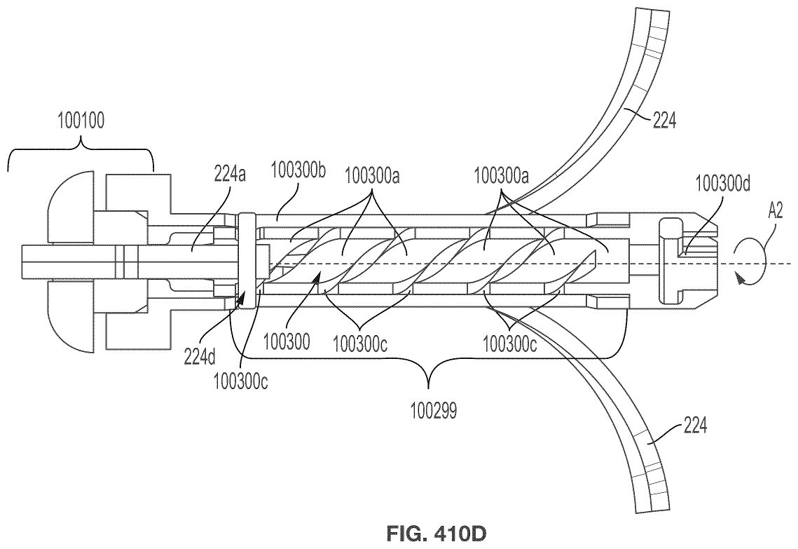

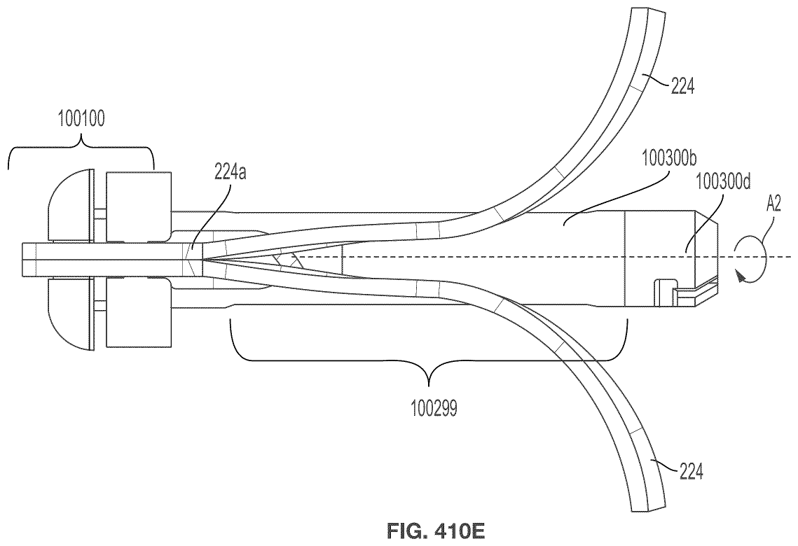

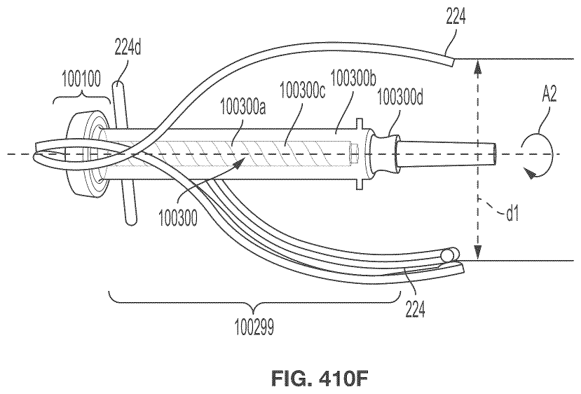

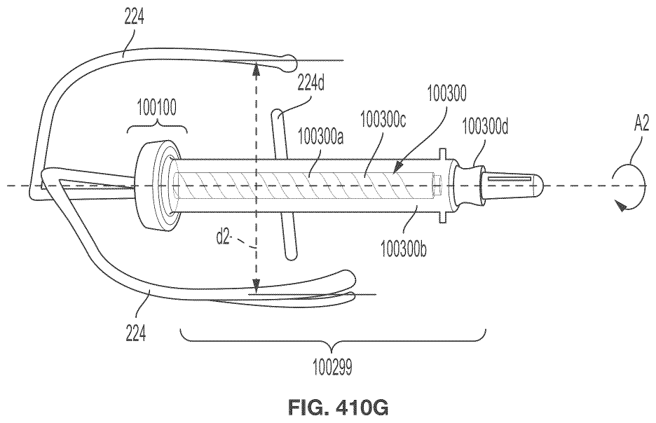

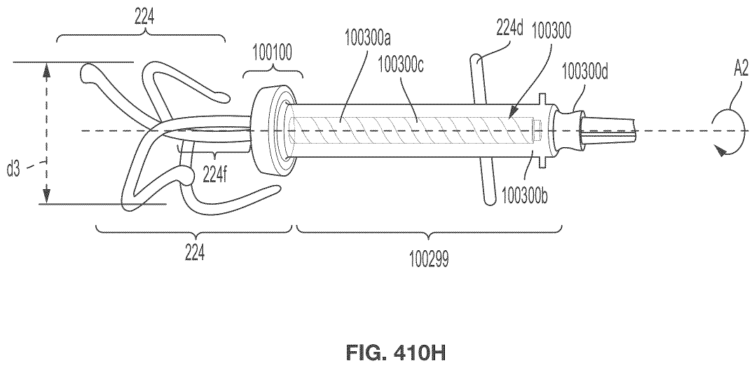

FIGS. 410A-410E illustrate an example implementation of a paddle narrowing and widening adjustment mechanism 100299 that automatically holds the paddle frames 224 in the adjusted position when the mechanism is released. The mechanism includes a coupler, such as the illustrated rotational member 100300 (e.g., spiral cut tube, etc.). In addition to adjusting the width of the paddles, linear movement of the entire mechanism 100299 can be used to open and close the paddle frames with an actuation element 212, as shown in FIGS. 30-36. One advantage of the rotational member 100300, is that the member can hold a paddle width without the use of a separate locking mechanism. Another advantage is that the rotational member 100300 can allow precise control of the paddle frame 224 width.

The mechanism 100299 is a helical screw system that extends the paddle frames 224 by moving the paddle frame inner ends or portions 224a along the axis A2. The details of this mechanism are discussed below. The motion of rotational member 100300 can be driven by components located in the proximal portion 100300d of the device. It is to be understood that the configuration shown in FIGS. 410A-410E are merely examples of some implementations. Variations in locations of components and specific construction are possible and within the scope of this disclosure. In addition, the cap 100100 is shown in FIGS. 410A-410E for illustrative purposes. It is also to be understood that other variations can use different the caps and/or other components. For example, the cap 100200 can alternatively be used in place of the cap 100100.

FIGS. 410A and 410C are cutaway, cross-sectional views of the mechanism 100299 to illustrate how it moves the paddle frames 224. As shown in FIGS. 410A and 410C, member 100300 includes a helical portion 100300a. The cutaway views of the helical portion 100300a in FIGS. 410A and 410C show six sections (the helical portion 100300a can have any number of sections). This is an artifact of the cutaway view. In fact, each of the six sections of helical portion 100300a are joined as a solid, continuous ribbon of material that makes a helical shape. The formation of the helix creates a slot 100300c in between each of the six sections. The helical portion 100300a is surrounded by a receiver, such as the illustrated case 100300b. The helical portion 100300a is rotatable about axis A2 as shown in FIGS. 410A-410C, while the case 100300b remains fixed. The helical portion 100300a, a slot 100300c, and the case 100300b are shown in in an exterior (as opposed to in cutaway) view in FIG. 410B.

FIG. 410A shows that the inner end or portion 224a of the paddle frame 224 interacts with the helical portion 100300a via a protrusion 224d, such as a post. In FIGS. 410A and 410C, the protrusion 224d points from portion 224c into/out of the page. FIG. 410B shows the protrusion 224d extending away from the inner end or portion 224a of the paddle frame 224 to which it is attached. As shown in FIG. 410B, the protrusion 224d fits into the helical slot 100300c in the helical portion 100300a and a linear slot 100300e in the case 100300b. The case 100300b is fixed with respect to rotation of the helical portion 100300a. Therefore, rotation of the helical portion 100300a about the axis A2 causes the protrusion 224d to move in a way that is guided by both slot 100300c and slot 100300e. In particular, when the helical portion 100300a is rotated around the axis A2, the slot 100300c pushes the protrusion 224d (and therefore the paddle frame inner end or portion 224a) along the 100300e and therefore along axis A2. The direction in which the protrusion 224d moves along the axis A2 (i.e., either towards the cap 100100 or in an opposite direction towards the proximal portion 100300d) depends on the direction of rotation of helical portion 100300a. As discussed above, motion of protrusion 224d causes the paddle frames 224 to either be drawn into or pushed out of the cap 100100 depending on the direction of rotation. In some implementations, whenever rotation of the helical portion 100300a stops, the mechanism 100299 holds the paddle frames 224 at the corresponding width.

Mechanisms for actuating the rotation of helical portion 100300a can include, for example, a user rotated rod, handle, or other fixture. The rotation can be manually activated, electronically activated, and/or controlled via software/computer interface. Other implements can include using a stepper motor, remotely or locally controlled, and/or any other suitable actuator. Such mechanisms can be coupled to the proximal portion 100300d in a wide variety of different ways, such as by any of the coupling arrangements disclosed in the present application. In some implementations, the coupling to the proximal portion 100300d facilitated both rotation of the helical portion 100300a to adjust the width of the paddle frames and linear extension of the entire mechanism 100299 relative to a delivery catheter and/or a coaptation element to open and close the paddles of the device.

FIGS. 410D and 410E show the rotation of helical portion 100300a from a side of the device. In both FIGS. 410D and 410E, the paddle frames 224 are in a widest position. FIG. 410D shows a cross-sectional view of helical portion 100300a. FIG. 410E shows this the paddle frame configuration, but with an exterior view of housing 100300b. As is shown in FIG. 410D, the protrusion 224d extends through the paddle frame inner ends or portions 224a, through the helical slots 100300c of the helical portion 100300c, and through the elongated slot 100300e of the housing 100300b.

FIGS. 410F, 410G, and 410H show portions of the paddle frames 224 in three different width positions (e.g., three different amounts of the paddle frames drawn into the mechanism 100299). The different positions are created in succession by rotating the helical portion 100300a about axis A2 in order to move protrusion 224d from distal (FIG. 410F) to more proximal (FIG. 410H) positions.

FIG. 410F shows the beginning of the movement with the protrusion 224d close to the cap 100100 at the distal end of member 100300. As shown in FIG. 410F, in this position both sets of the paddle frames 224 are in the widest position. The two paddle frame portions 224 are a distance d1 from one another. As the helical portion 100300a is rotated around axis A2, the slot 100300c pushes the protrusion 224d toward the proximal end of rotating member 100300 (e.g., toward end 100300d).

Continuing this rotation results in the reduced amount of the paddle frames 224 extending from the cap 100100 shown in FIG. 410G. In FIG. 410G, the protrusion 224d has now been moved to a middle position between proximal and distal portions of the rotating member 100300. Correspondingly, the paddle frames 224 now have partially narrowed. In this partially extended position, the two paddle frames 224 have a distance d2 between each other. The paddle frames 224 can be configured such that the distance d2 is greater than the distance d1 (e.g., the paddle frame portions move apart as they are narrowed by retraction into the mechanism) or such that the distance d2 is less than the distance d1 (i.e. the paddle frame portions move toward one another as they are narrowed by retraction into the mechanism).

Continuing the rotation of helical portion 100300a around axis A2 further pushes protrusion 224d towards the proximal end of member 100300 (i.e., toward 100300d). The result is shown in FIG. 410H. The paddle frames 224 are further narrowed by retraction into the mechanism 100299. In this position, the two paddle frames 224 have a distance d3 between each other. The paddle frames 224 can be configured such that the distance d3 is greater than the distance d2 (e.g., the paddle frame portions move apart as they are narrowed by further retraction into the mechanism) or such that the distance d3 is less than the distance d2 (i.e. the paddle frame portions move toward one another as they are narrowed by further retraction into the mechanism).

In some implementations, the paddle frames 224 are actively narrowed and passively expanded. As such, the expanded condition can be the natural or substantially unstressed shape of the paddle frames. To move the paddle frames to the narrowed condition the paddle frames are stressed to flex the paddle frames from the expanded state to the narrowed state. This stress can be concentrated in certain areas of the paddle frames 224, such as at the area where the paddles enter the cap. As described above, one way of reducing this stress is to provide a radiused or tapered entry for the paddles into the cap.

In some implementations, the paddle frames are structurally modified to reduce the stress in the area where the paddle frames enter the cap. The paddle frames can be structurally modified to reduce the stress in the area where the paddle frames enter the cap in a variety of different ways. For example, the area where the paddle frames enter the cap can be moveably connected to the remainder of the paddle frame, the area where the paddle frames enter the cap can be decoupled from the remainder of the paddle frame, the area where the paddle frames enter the cap connected to the remainder of the paddle frame by a flexible component, etc.

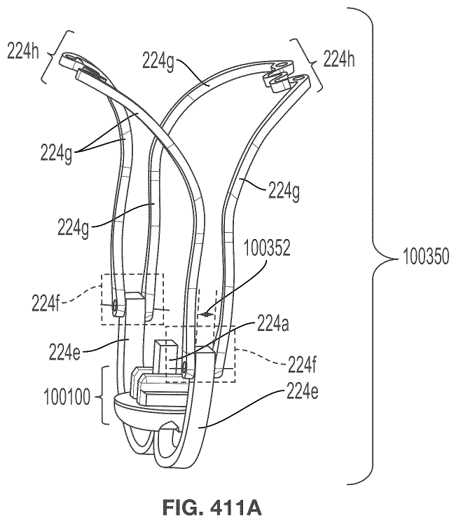

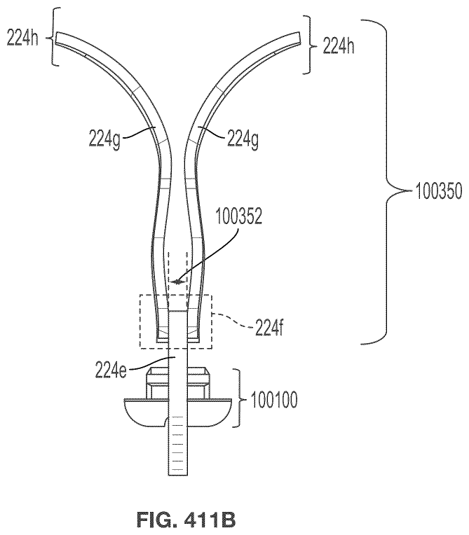

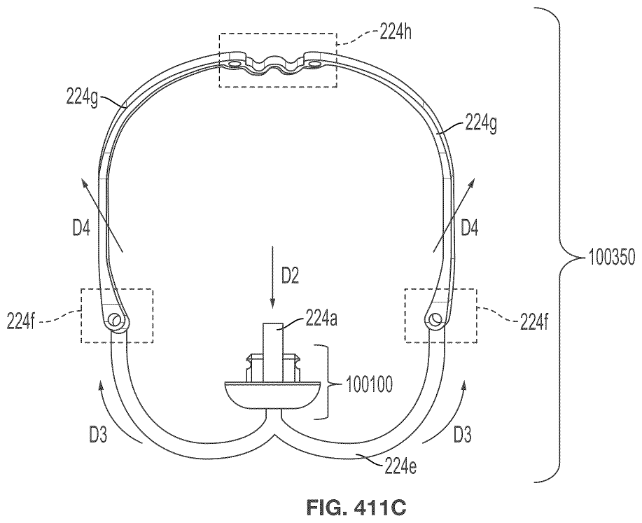

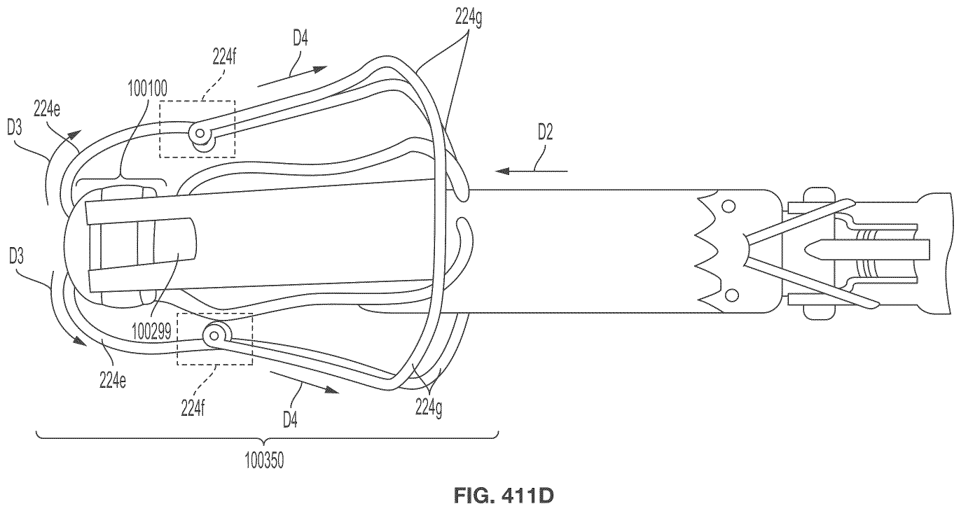

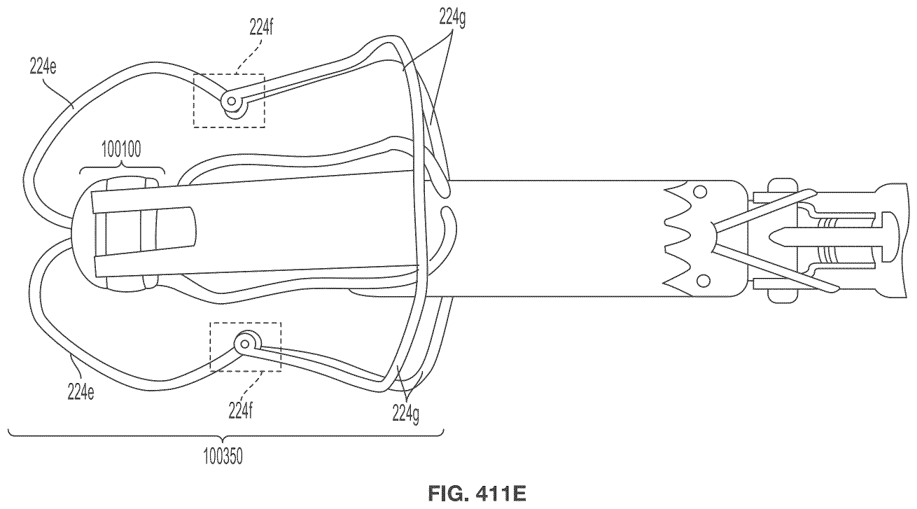

FIGS. 411A-411E show a paddle system 100350 that addresses the problem of stress concentration at the portions of the paddle frames 224 that are pulled into the cap, in part, by segmenting the paddle construction. The paddle system 100350 comprises a connector 224e (e.g., shaped metal component, shaped plastic component, tether, wire, strut, line, cord, suture, etc.) and an upper portion comprising outer paddle frame portions 224g joined by a pivot point 224f. Although FIGS. 411A-411E show the system 100350 being used in conjunction with the cap 100100, it is to be understood that this is merely by way of example. System 100350 can be used in conjunction with other components disclosed and/or implied herein, including for example the cap 100200.

FIGS. 411A-411C show different views of the system 100350. FIGS. 411A-411C show the pivot point 224f in the form of a hinge. A hinge would allow the user to actively increase the paddle frame 224 width by pushing the inner end or portion 224a in direction D2 (FIG. 411C) and to decrease the paddle width by pulling the inner end or portion 224a in the opposite direction. More specifically, pushing the inner end or portion 224a in the direction (e.g., moving protrusion 224d (FIG. 410F)) pushes connector 224e along direction D3, while the connector 224e and the outer paddle frame portions 224g are free to pivot relative to one another about the pivot point 224f. In turn, this causes the outer paddle frame portions 224g to bow along direction D4, thus expanding the paddle frame 224. Pulling the inner end or portion 224a in the opposite direction pulls the connector 224e into the cap 100100, while the connector 224e and the outer paddle frame portions 224g are free to pivot relative to one another about the pivot point 224f. In turn, this causes the outer paddle frame portions 224g to bow inward (opposite direction D4), thus narrowing the paddle frame 224.

In some implementations, the bowing of the outer paddle frame portions 224g is resisted and/or provided with a countervailing restoring force by resilient element or restoring member 224h (e.g., spring, etc.). Resilient element or restoring member 224h can allow the active narrowing or widening of the paddle frame 224 width to be automatically or passively reversed. The resilient element or restoring member 224h can include a spring, as shown in FIGS. 411A-411C. However, it is to be understood that other variations can include other types of biasing mechanisms (e.g., leaf springs, coil springs, etc.), or any other restoring mechanism described herein can be used.

As shown in FIGS. 411A and 411B, the positioning of the connector 224e between the outer paddle frame portions 224g at the pivot points 224f of the system 100350 creates a spacing 100352 between adjacent outer paddle frame portions 224g of the paddle frame 224. The spacing 100352 can prevent, inhibit, or reduce the outer paddle frame portions 224g from pinching or restricting the valve leaflets of the valve being repaired. That is, the spacing 100352 can reduce pinching of the free edge of the leaflet between outer paddle frame portions 224g. The spacing 100352 can be adjusted based on the selection and fabrication of connector 224e (e.g., their thickness) at the pivot point 224f.

Using the pivot point 224f to connect a segmented upper frame portion 224g of the paddle frame 224 and the connector 224e can facilitate certain manufacturing advantages. Segmenting the upper paddle frame portion 224g and the connector 224e allows these components to be fabricated from different materials and/or via different methods. For example, the connector 224e can be fabricated by stamping or laser cutting a ribbon of more flexible material and/or a stronger material that can withstand the application of higher strains, while outer paddle frame portions 224g can be fabricated using a less flexible and/or weaker materials. The outer paddle frame portions 224g can be made of a less expensive material, such as a bent wire.

FIGS. 411D and 411E show an example of a device or valve repair device or implant that includes the hinged paddle system 100350. The valve repair device or implant can include any of the features of any of the other devices or implants disclosed in the present application. By comparing FIGS. 411D and 411E, the user moves ends of the paddle portions in the direction D2, for example out of a cap 100100 with a paddle width control mechanism 100299. This pushes connector 224e along direction D3. That motion of connector 224e, then actuates outer paddle frame portions 224g, via pivot point 224f, to move along D4.

FIG. 411E shows a result of this motion. As shown in FIG. 411E, both connector 224e and outer paddle frame portions 224g have been extended to widen the paddle frames. As discussed above, this motion extends the paddles 224 with diminished stress concentration on the system 100350. Note that, although optional restoring member 224h is not shown in FIGS. 411D and 411E, it is to be understood that restoring member 224h can be included. If so, restoring member 224h can create a mechanical bias toward returning the paddle frame 224 extent to its original fully widened or fully narrowed position.

As discussed above, there are advantages to fabricating portions of the disclosed the devices (e.g., device/implant 200) out of bulk materials, such a sheet material, such as a sheet of metal or plastic, rather than from a braided or woven networks of wire. Braided or woven networks can facilitate flexibility of design by which the device can elongate and compress into a tracking condition to enable delivery through a delivery system and intraprocedural maneuvering and expand into the shape of the device or implant. However, devices made from a braided or woven network of wires can be expensive to manufacture. In some implementations, portions of the valve repair device or implant can be made from a flat sheet of material. For example, coaptation element supports, inner paddle, and/or a paddle frame connection portion can be made from a flat sheet of material.

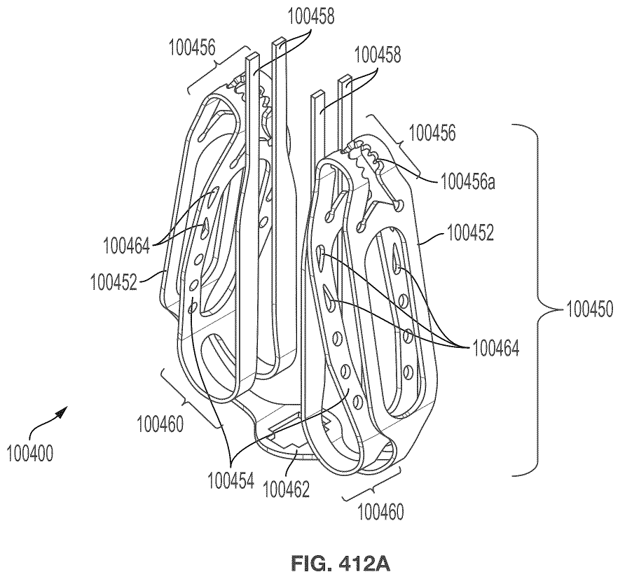

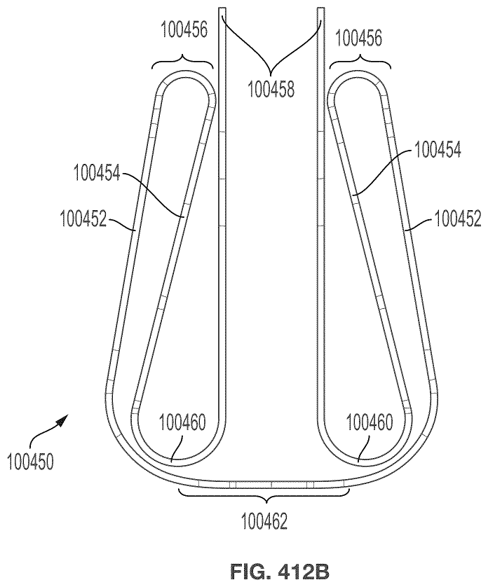

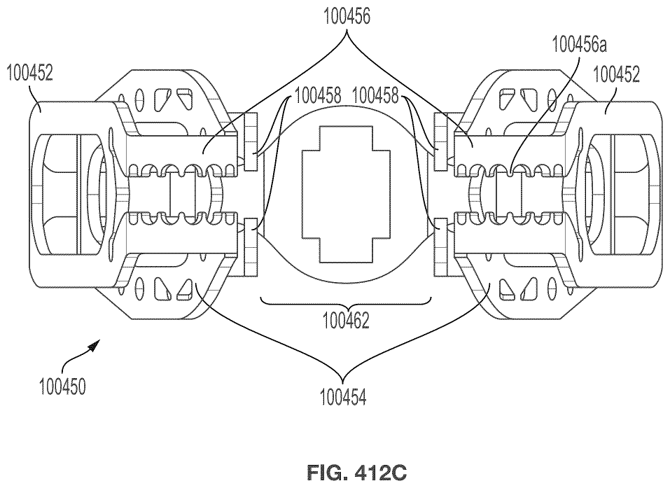

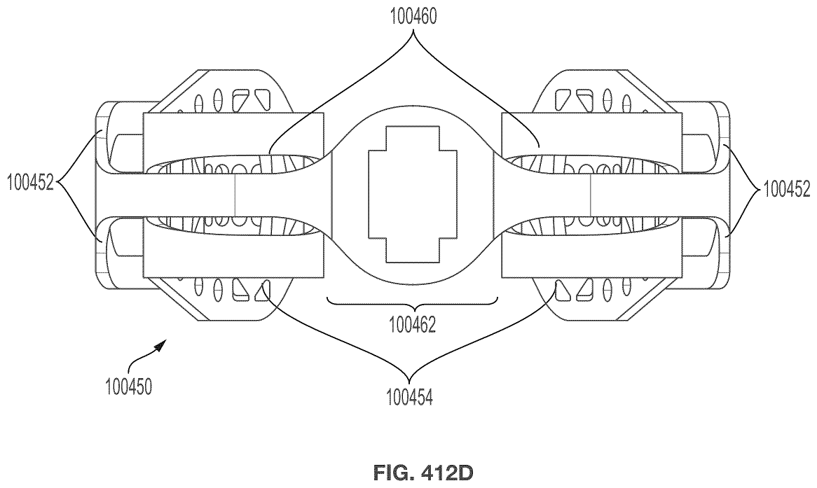

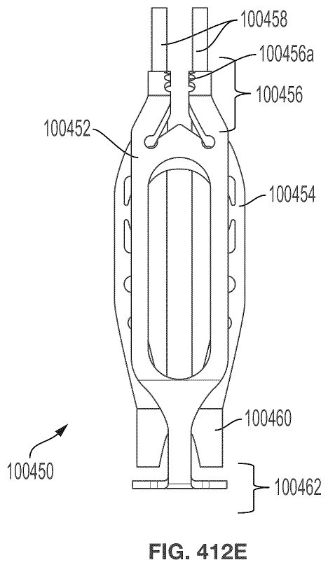

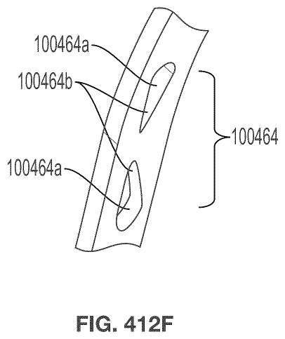

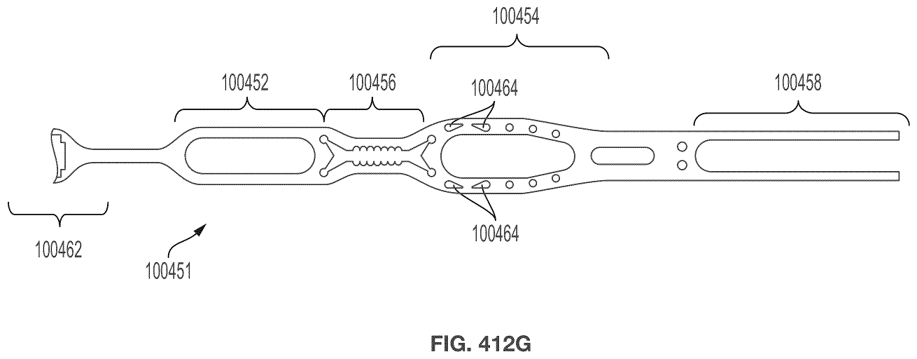

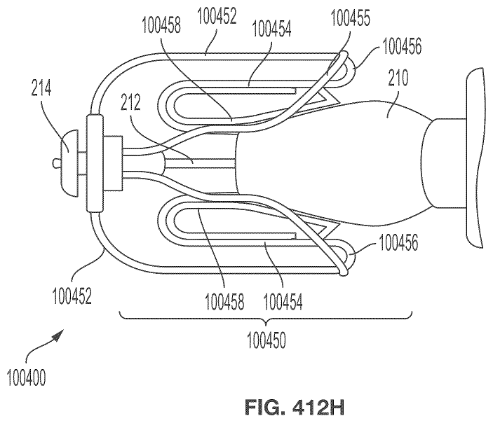

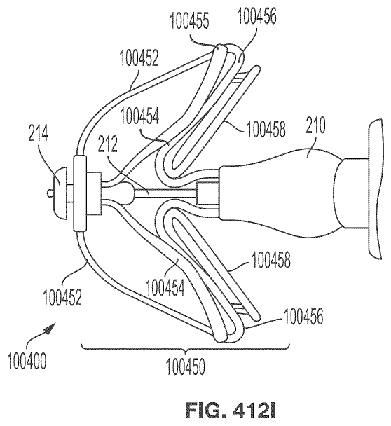

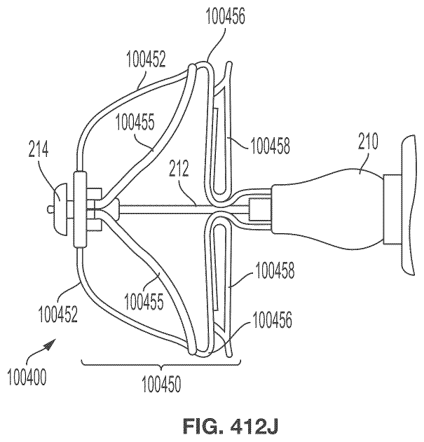

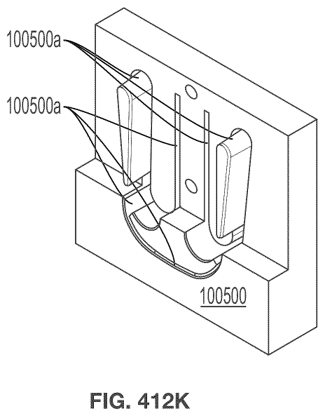

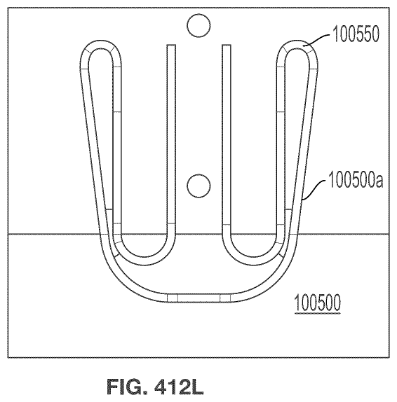

FIGS. 412A-412L show a paddle structure 100450 in which a braided or wire the paddle structure is replaced by a structure 100450 fabricated from a sheet of material. In the illustrated example, the paddle structure 100450 is made from a single, contiguous piece of material (e.g., a nitinol flat sheet or strip of material that can be laser cut, photo etched, or stamped as a flat part and subsequently shaped). The precise material and manufacturing method can vary.

Comparison of FIG. 412A with FIG. 23 shows that the paddle structure 100450 takes a similar form as the paddle structure of FIG. 23. Table 1 below compares components in the paddle structure 100450 with functionally similar components in braided or woven variation shown in FIG. 23. The components in the paddle structure 100450 are shown in a perspective view in FIG. 412A, side view in FIG. 412B, top view in FIG. 412C, bottom view in FIG. 412D, and another side view in FIG. 412E.

TABLE 1
Correspondence between components in FIGS. 23 and 412A-412E.

| | The paddle structure | Braid or woven the |
|---|---|---|
| | 100450 in FIGS. | paddle structure in |
| Component | 412A-412E | FIG. 23 |
| Outer the paddle | 100452 | 220 |
| Inner the paddle | 100454 | 222 |
| Inner/outer the paddle | 100456 | 223 |
| connection portion | | |
| Moveable arm | 100458 | 234 |
| Joint portion | 100460 | 238 |
| Cap/the paddle | 100462 | 221 |
| connection portion | | |
| Paddle Frame | 100455 | 224 |

The components of the paddle structure 100450 operate substantially similarly to their functional equivalents identified in Table 1. That is, the descriptions of the functional equivalents in the context of FIGS. 30-37 apply equally well to the corresponding components in the paddle structure 100450.

As shown in FIGS. 412A-412E, inner/outer the paddle connection portion 100456 can be implemented by a cutout and series perforations 100456a. Perforations 100456a allow connection portion 100456 to flex through a range of movement for opening and closing of the paddle structure 100450 shown in more detail below with respect to FIGS. 412H-412J. Joint portion 100460 can have a similar structure, though it is shown in FIGS. 412A-412E without perforations. More generally, either connection portion 100456 or joint portion 100460 can be fabricated in any suitable manner that creates flexibility to allow opening and closing of the paddle structure 100450.

A cap/paddle frame connection portion 100462 in FIG. 412A connect the paddle structure 100450 to a distal the cap, such as the cap 214 and the paddle frames 224. The cap/paddle connection portion 100462 can take on a number of suitable forms. The connection portion 100462 is illustrated from above in FIG. 412C and from below in FIG. 412D. The connection portion 100462 can have any suitable configuration that fixes the paddle structure 100450 to the cap and/or the paddle frames. In the illustrated example, the connection portion includes a cutout that facilitates a snap fit connection of the paddle frames and/or the cap.

Turning back to FIG. 412A, each the paddle structure 100450 can contain eyelets 100464 that can be used to attach a cover and/or other components to the paddle structure 100450. Eyelet structure 100464 is shown in more detail in FIG. 412F. One of the purposes of the eyelets 100464 is to anchor sutures that connect the cover and/or other component sufficiently so that the suture does not pull out of the eyelet as stitching of the cover or other component to the paddle structure is started. In particular, the suture that is used to stitch the cover or other component to the paddle structure can be inserted into a wider portion 100464a of the eyelet 100464 that is wide enough to accommodate the entire diameter of suture. Then the suture can be anchored to the eyelet 100464 by moving suture from the wider portion 100464a to the narrower portion 100464b. The narrower portion 100464b has a width that is considerably less than the width of the wider portion 100464a. As a result, the narrower portion 100464b squeezes and fixes the suture into place. That is, the suture is wedged in the narrower portion 100464b.

FIG. 412G shows is a plan view of one-half of the flat, cut sheet material 100451 that is used to make the paddle structure 100450. FIG. 412G illustrates the location of the eyelets 100464 with respect to the inner/outer the paddle connection portion 100456 and other portions of the paddle structure 100450.

FIGS. 412H-412J show an example opening and closing motion of the paddle structure 100450 when used in a device or implant. The paddle structure 100450 can have the range of motion of any of the paddle structured disclosed herein. For example, the paddle structure can also be moved to an extended position and can have the same or similar range of motion as the paddle structure that is illustrated FIGS. 23 and 30-37. The valve repair device or implant that includes the paddle structure 100450 can take a variety of different forms and can include any of the features of any of the devices or implants disclosed herein.

The position of the valve repair device or implant shown in FIG. 412H is a fully retracted position that corresponds to the fully retracted position shown for the example illustrated in FIG. 23. Referring to FIG. 412I, the actuation element 212 extends the cap 214 away from the coaptation element 210 to partially open the paddle assembly. The position shown in FIG. 412I corresponds to the partially open position shown in either FIG. 30 or FIG. 31. Referring to FIG. 412J, the actuation element 212 further extends the cap 214 further away from the coaptation element 210 to further open the paddle assembly. The position shown in FIG. 412J corresponds to the laterally extended or open position shown in FIG. 32.

FIGS. 412K and 412L show a die 100500 that can be used to press the paddle structure 100450 from a single strip or piece of metal. A single strip of metal (not shown) can be placed in the accommodating portion 100500a shown in FIG. 412K. As shown in FIG. 412K, accommodating portion 100500a is fashioned to provide a mold or press with the shape of the paddle structure 100450 (see, e.g., FIG. 412B). FIG. 412L shows a profile of a strip 100550 in the accommodating portion 100500a being molded or pressed. Any number of suitable techniques can be used to shape the strip 100550 while in die 100500, including applying heat, pressure, and/or chemical treatment. Strip 100550 can be laser cut to include perforations, the holes, eyelets and other structures prior to adding it to die 100500. For example, a pre-cut, flat strip can resemble the structure 100450 shown in FIG. 412G.

As discussed above, the devices (e.g., device/implant 200) can be used to navigate dense chordal structures during both mitral and/or tricuspid procedures. Such chordal structures present challenges during the device placement as they can be difficult to visualize sufficiently for precise navigation with current remote image modalities (e.g., remote fiber optic cameras and/or sensors). Often the user must rely, at least partially, on the tactile sensation of resistance or inability to seat the device (clasp the leaflet) because of unseen or not fully visualized chordal structures. Sometimes these indications of chordal interactions are not sensed until after the device is deployed or closed. Chordal interactions can be indicated by a substantial amount of regurgitation after closing the device. Because seating the device properly is important to its function, a more straight-forward and accurate method of sensing chordal interaction would be advantageous. In particular, an accurate visual indication of chordal interaction could reduce the time needed to grasp the leaflets and serve as a predictor for residual regurgitation prior to the device closure.

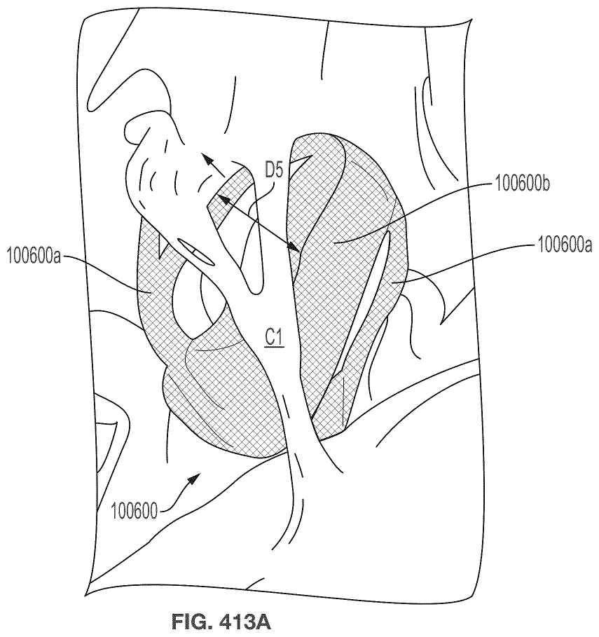

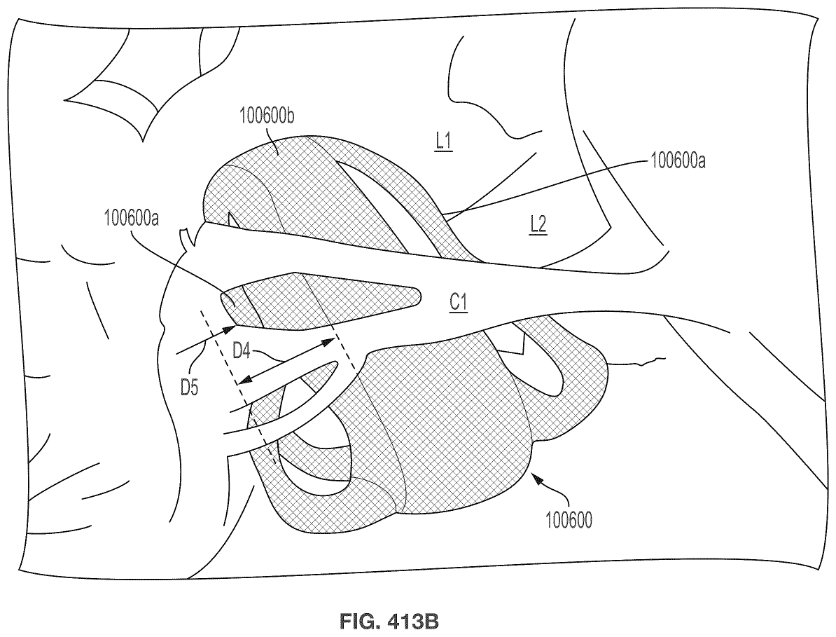

FIGS. 413A and 413B show a device 100600 with paddles 100600a that are passively narrowed by being pressed on and passively expand back to their original state when the paddles are no longer being pressed on. In the example illustrated by FIGS. 413A and 413B, the paddles 1005600A are designed to provide a visual indication of an interaction with chordal or other biological structures. Specifically, the device 100600 is relatively wide and flexible paddles 100600a bend to provide a visual indication of such interaction (e.g., interactions with chordae tendineae). The paddles 100600a have a medial-lateral hoop strength low enough to allow inward deflection (i.e., toward the center 100600b of the device) when pressing against chordae tendineae or other obstructions.

FIG. 413A shows the device 100600 properly seated and without interfering mechanical interaction with chordae tendineae C1. Even still, there may be some interaction between the device 100600 and chordae tendineae C1. In particular, chordae tendineae C1 may push or pull the flexible paddles 100600a along direction D5. However, as shown in FIG. 413A, the interaction of the paddles 100600a with chordae C1 is insufficient to substantially bend the paddles 100600a and potentially disrupt operation of the device 100600. Visual inspection of the device 100600 embedded in this configuration via remote camera would readily show that the paddles 100600a are in their native configuration (i.e., have not bent substantially along direction D5). Therefore, a user could readily ascertain by visual inspection that the device 100600 is unobstructed.

In contrast, FIG. 413B shows a case in which the paddles 100600a have significantly interacted with chordae tendineae C1. The interaction causes the paddles 100600a to laterally deflect (d4) along direction D5 towards the center 100600b of the device. As shown in FIG. 413B, the device 100600 is not in an optimal position on the leaflets L1 and L2. The lateral deflection d4 can be perceived relatively easily in a visual image of the device 100600 taken by remote camera. The deflection d4 causes a visible distortion of the paddle 100600a shape. That distortion can be visible regardless of whether chordae tendineae C1 are readily visible. If so, this improves accuracy and sensitivity to the user detection of the status of the device 100600. Moreover, the user could likely tell where interaction is occurring based on the visual indication. If the user sees the interaction while attempting to capture a leaflet (e.g., the leaflets L1 and L2), the user perceives the location of the obstruction and maneuver the device 100600 to reduce interaction. In addition, a visual indication of chordal interaction can also serve as a predictor for residual regurgitation prior to the device closure and/or release from the delivery system.

Suitable flexibility in the paddle 100600a can be achieved in a variety of different ways. The paddles 100600a can be fashioned from shape memory alloy with increased flexibility. Also or alternatively, the paddles 100600a can be fashioned with a hinge or pivot element (e.g., pivot point 224f shown in FIG. 411C) allowing lateral flexibly. Other structures, such as hinges or rotating joints can also be employed to similar effect. Other methods to impart suitable flexibly can include laser cut patterns, including perforation (e.g., as shown for connection portion 100456 in FIG. 412A), using anisotropic materials, and/or using a laminated material. Each the paddle 100600a can have compliant and/or flexible components or sections. For example, the compliant portion can be located on the lateral side of the paddle 100600a (distant from central portion 100600b). A less flexible portion can be located on the medial side (closer to central portion 100600b).

An important aspect of this disclosure is that the flexible the paddles 100600a can be fashioned in such a way that they are still able to operate under compromised conditions. In some implementations, the distortion of the paddle 100600a shape accommodates use of the device 100600 where interaction with chordae tendineae C1 is unavoidable. The device 100600 is implanted and the paddle 100600a flexes. This flexing allows the implanted device 100600 to function as intended where similar devices with rigid paddles would not be able to properly function due to the interaction with the chordae tendineae C1. That is, the flexing of the paddle 100600a accommodates the chordae tendineae C1 or conforms to the chordae tendineae to allow the device 100600 to operate as intended.

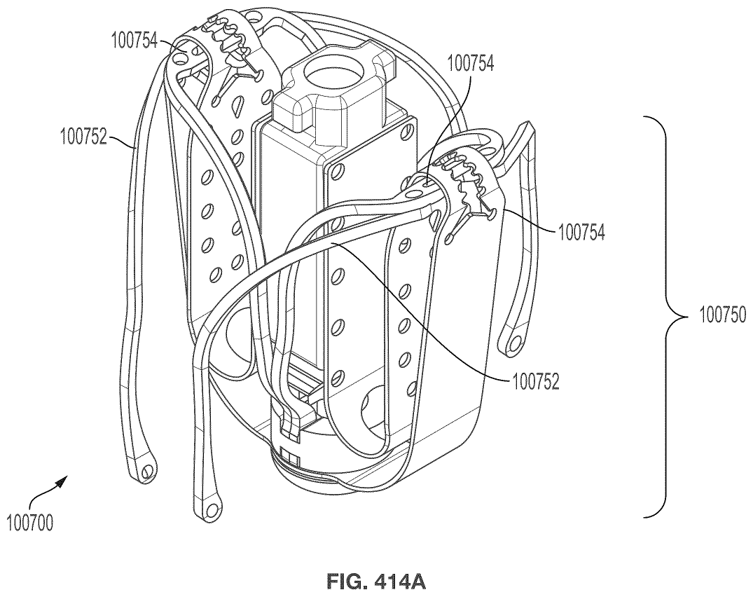

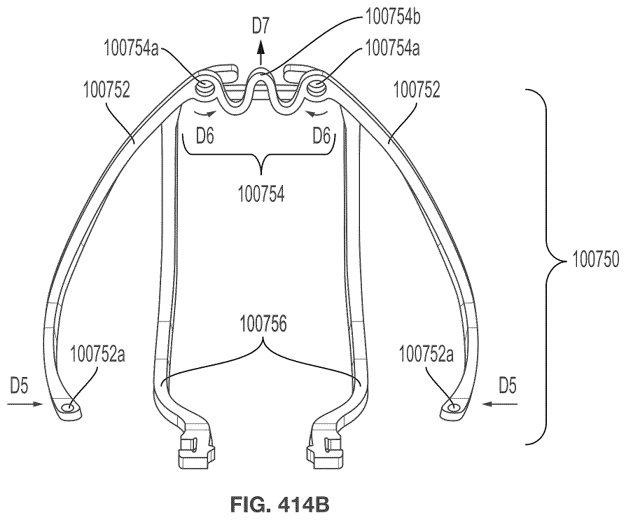

FIGS. 414A-414B FIGS. 413A and 413B show another device 100700 with outer paddle frames 100752 that are passively narrowed by being pressed on and passively expand back to their original state when the paddle frames are no longer being pressed on. Referring to FIG. 414B, the outer paddle frames 100752 include a restoring component 100754, such as a spring portion, that can passively assist restoring of the outer paddle frame 100752 (shown in FIGS. 414A and 414B) to its full width after the outer paddle frame 100752 has been flexed inward along direction D5 (FIG. 414B). In addition, the outer paddle frame 100752 can be fashioned out of a flexible material (e.g., shape memory alloy, nitinol, CuAlNi, NiTi, and various alloys of Zn, Cu, Au, and Fe, etc.) that does not substantially plastically deform the outer paddle frame 100752 during narrowing.

More particularly, referring to FIG. 414B, the outer paddle frame 100752 can be passively narrowed by engaging an obstacle, such as the chordae tendineae, that applies a force on an outer frame portion 100752a the paddle frame 100752 in the direction D5 (see, e.g., displacement D5 due to chordae tendineae C1 in FIG. 413B) can press outer the outer paddle frames 100752 along direction D5.

In any case, force can be communicated to restoring component 100754 along direction D6 via length of the outer paddle frame 100752 and joint mechanism 100754a. The communicated force then pivots the outer paddle frame 100752 about the pivot connection 100754a and compresses the center portion 100754b and/or introduces a displacement of the center portion 100754b of the restoring component 100754 along direction D7. Compression and/or displacement of center portion 100754b stores energy as a restoring force that can be used to move outer the outer paddle frame 100752 back to its original shape and configuration. Once the force causing displacement along D5 ceases (e.g., the device 100700 moves clear of an interaction with biological material), outer the paddle frame 100752 can tend to return to its original shape. Center portion 100754b of the restoring component 100754 assists this process by applying a restoring force in the direction opposite to D7. The restoring force is then transmitted to the outer paddle frame 100752, causing it to flex in the opposite direction as D5. Subsequently, outer the paddle frame 100752 returns to its original shape, as shown in FIGS. 414A and 414B.

Another advantageous aspect of the paddle configuration 100750 is that the outer the outer paddle frames 100752 can still be opened and closed even during a deflection of the end(s) along the direction D5. That is to say, the outer paddle frames 100752 can be made of substantially stiff material such that a deflection along D5 does not prevent the user from opening or closing the paddles. For example, the paddles can still be opened and closed even when there is a substantial obstruction or interaction with biological material (e.g., displacement d1 due to chordae tendineae C1 in FIG. 413B).

Although the restoring component 100754 shown in FIGS. 414A and 414B as an integral spring, it is to be understood that any suitable restoring force mechanism can be used. Examples include coiled wire, such as a compression spring or other similar the device, shape memory alloys, pneumatic devices, and other elastic the devices can all be used as the restoring component 100754. The restoring component 100754 can further include material cut in a patterned geometry that reduces strain during stretching or can include a polymer (e.g., rubber or elastomeric polymer). Rings or bands of polymer or other elastic material can be used. Still other examples of materials that could be used in restoring component 100754 include super-elastic nitinol, other nitinol, and/or stainless steel. Preferably, the material is biologically inert. In the illustrated example, the restoring component 100754 can be attached in a laminated configuration over the narrow inner paddle frames 100756.

The outer paddle frames 100752, the inner paddle frames 100756, the restoring component 100754, can be constructed using laser cutting, die casting, 3D printing, or other advanced manufacturing techniques. These components can be fabricated separately and assembled when finished. Such a manufacturing technique can be amenable to simple, scalable, mass production.

Even after some valve repair devices or implants are installed in a native valve some regurgitation can still occur. During systole, for example, blood may flow around the device where coaptation of the native leaflet adjacent to the device (e.g., the leaflets 20 and 22 adjacent to the coaptation portion 210 of the device in FIG. 53) is not complete. In FIG. 53, this would correspond to blood flow around coaptation element 210 at the medial aspects 201, 203. The location of medial aspects 201, 203 of the coaptation element 210 in the device 200 prior to deployment are shown in more detail in FIG. 51. It would be advantageous to add an element that would stop, plug, or deter blood flow around portions of the device that the leaflets may fail to fully coapt around.

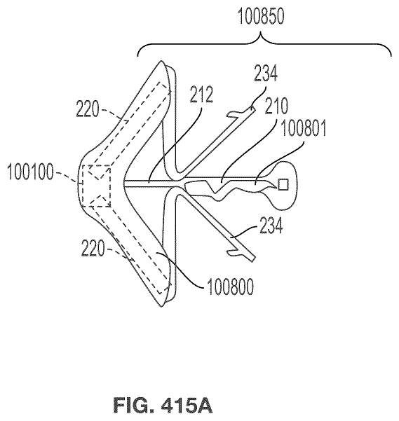

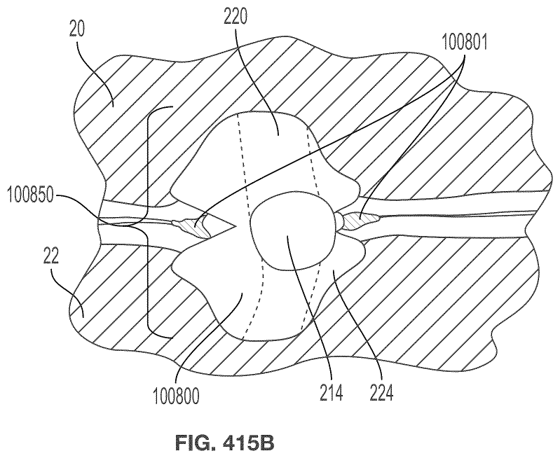

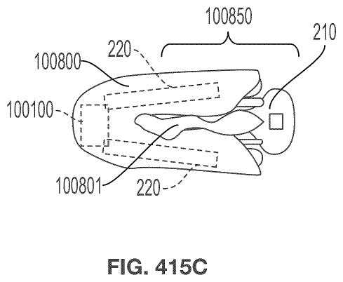

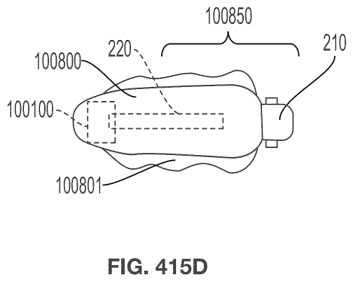

FIGS. 415A-415D show an example valve repair device or implant 100850 that includes one or more fabric extensions 100801 that can decrease blood flow around a deployed the device. FIG. 415A shows the one or more fabric extensions 100801 in use during deployment of the device 100850 in an open mode, corresponding to FIG. 53, as described above. The device 100850 includes an extendable the cap 100100 (e.g., as shown in FIG. 411E). The cover 100800 covers both the cap 100100 itself as well as the connector 224e and the outer paddles 220 that extend beyond the cap (see FIG. 411E).

FIG. 415B is a view of the device 100850 the capturing the leaflets 20 and 22, as in FIG. 53. Comparing FIG. 415B to FIG. 53 shows that the one or more fabric extensions 100801 block the gaps occurring due to lack of coaptation (e.g., the gaps shown in FIG. 53 adjacent to the coaptation portion 210 of the device near medial aspects 201, 203). Therefore, the presence of the one or more fabric extensions 100801 can substantially reduce or inhibit, and may even prevent, regurgitation once the device 100850 is deployed.

FIGS. 415C and 415D show the cover 100800 while the inner and outer paddles 222, 220 are in the closed position. FIG. 415C is a side view corresponding to a view from the same direction as in FIG. 415A. FIG. 415D is a side view corresponding to rotating the device 100850 with respect to the view in FIG. 415C 90 degrees around coaptation portion 210 and towards the bottom of the page.

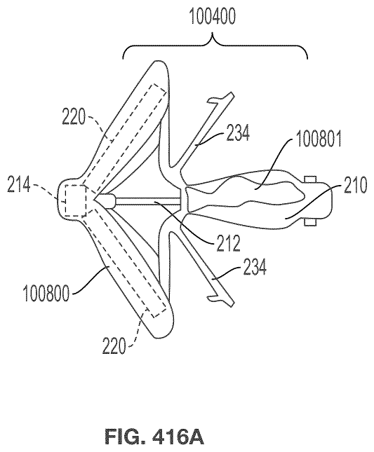

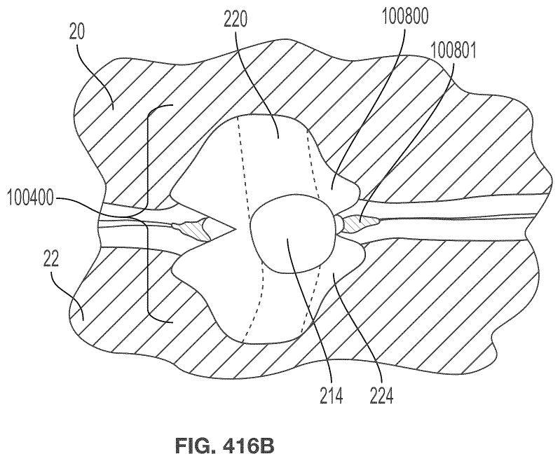

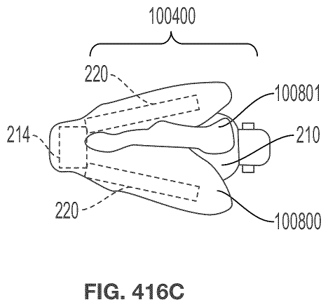

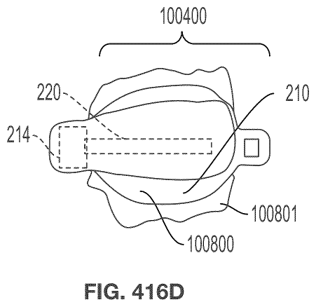

FIGS. 416A-416D show an example of a different valve repair device/implant 100400 with one or more fabric extensions 100801. The valve repair device/implant of 416A-416D has a larger coaptation element 210 than the device 100850 illustrated by FIGS. 415A-415D. FIG. 416A shows the one or more fabric extensions 100801 in use when the device 100400 is being deployed (i.e., in the open, capture ready position, corresponding to FIG. 53, as described above).

FIG. 416B is a view of the device 100400 the capturing the leaflets 20 and 22, as in FIGS. 53 and 415B. Similarly, as in FIG. 415B, the one or more fabric extensions 100801 has blocked the gaps occurring due to lack of coaptation (e.g., the gaps shown in FIG. 53 adjacent to the coaptation portion 210 of the device). Therefore, the presence of the one or more fabric extensions 100801 can substantially reduce regurgitation.

FIGS. 416C and 416D show the one or more fabric extensions 100801 while the inner and outer paddles 222, 220 are retracted. FIG. 416C is a side view corresponding to a view from the same direction as in FIG. 416A. FIG. 416D is a side view corresponding to a rotating the device 100400 90 degrees out of page with respect to the view in FIG. 416C.

The material used in the one or more fabric extensions 100801 can include any suitable material that is non-toxic, non-bio reactive, and is able to diminish blood flow. These include various woven fabrics or fibers, as well films and/or shields. The cloth, films, or shields can be made of synthetic materials (e.g., polymers), organic materials (e.g., organic fibers), mixtures of the two, and/or other materials. Materials with biological functionality can be included. Examples of the latter include materials that can accelerate or promote tissue regrowth and growth factors or nutrients.

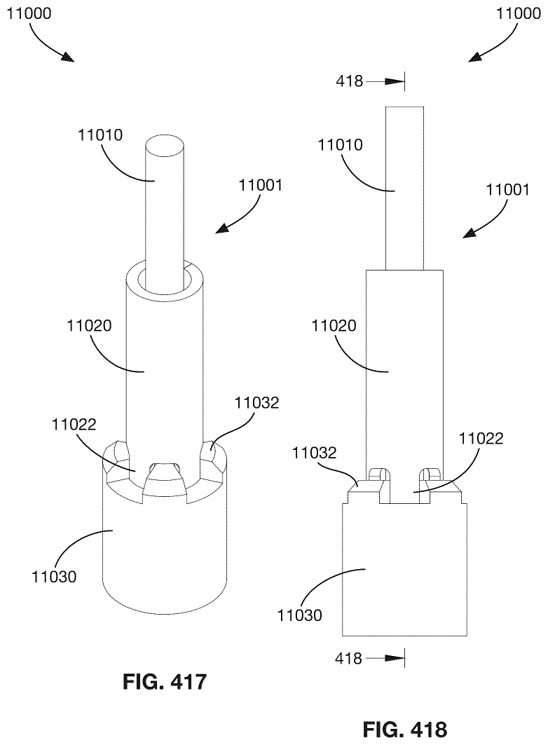

In some implementations of a device, a single actuation shaft or member can be actuated linearly and rotationally—i.e., the actuation shaft can extend and retract to apply force along the longitudinal axis of the shaft and can rotate clockwise and counter-clockwise to apply a torque to a component of the device. The device can include a mechanism that is actuated via rotational motion, such as, for example, a helical member or a threaded member that transforms rotational movement into linear movement to actuate a mechanism of the device. Some implementations of couplings that facilitate transmission of rotational movement of an actuation shaft or member to a mechanism of a device are shown in FIGS. 417-482.

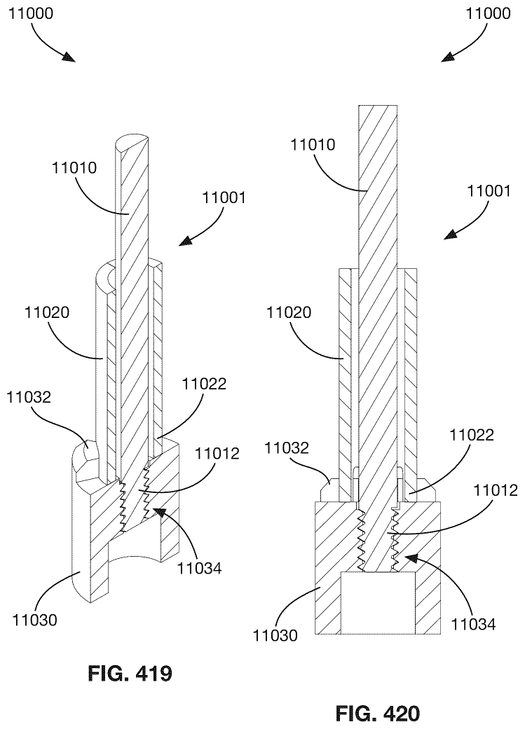

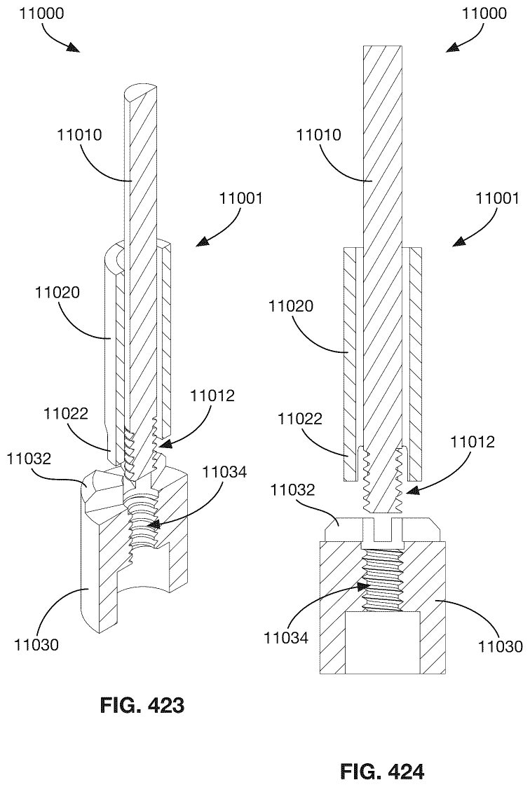

Referring now to FIGS. 417-424, an example coupling assembly 11000 for a device, such as any of the devices disclosed herein, is shown. The coupling assembly 11000 can be used to releasably couple to one or more positionable components of any of the implants or devices disclosed herein. For example, the coupling assembly 11000 can attach to a proximal end of an implant or device, can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein).

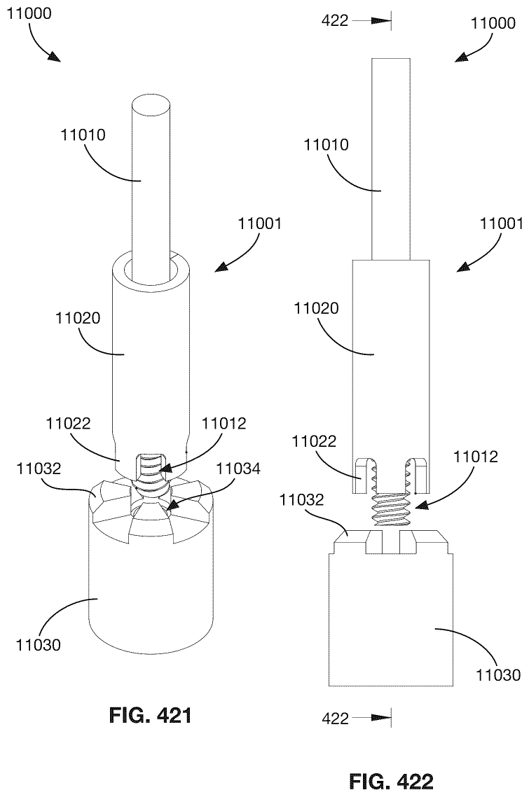

The coupling assembly 11000 is shown in a coupled condition in FIGS. 417-420 and in an uncoupled condition in FIGS. 421-424 as the coupling assembly is initially coupled during implantation of the device and is then uncoupled to disconnect an actuation member 11001 (e.g., shaft, tube, wire, etc.) from the device. The coupling assembly 11000 includes a retention member 11010, a distal gripper 11020 of the actuation member 11001, and a proximal head 11030 of a rotatable actuation member such as, for example, a helical or threaded member like the rotational member 100300 shown in FIGS. 410A-410E and described above. The retention member 11010 can be a pin, a wire (e.g., a nitinol wire), a shaft, or the like and includes a threaded end 11012 for engaging a threaded opening 11034 of the proximal head 11030. The threaded end 11012 and threaded opening 11034 can take on a wide variety of forms suitable for transmission of pushing and pulling forces transmitted through the retention member 11010. The distal gripper 11020 includes engagement protrusions or fingers 11022 for intermeshing with engagement protrusions or fingers 11032 of the proximal head 11030. The engagement protrusions or fingers 11022, 11032 can take on a wide variety of forms suitable for transmission of rotational forces between the distal gripper 11020 and proximal head 11030, such as, for example, fingers or protrusions having a different shape or number. The retention member 11010 applies a retention force to the proximal head 11030 via engagement between the threaded end 11012 and threaded opening 11034 to ensure the engagement protrusions or fingers 11022 of the distal gripper 11020 and the engagement protrusions or fingers 11032 of the proximal head 11030.

During implantation of the device, the distal gripper 11020 of the rotatable actuation member (e.g., shaft, tube, etc.) can be rotated to simultaneously rotate the proximal head 11030, thereby actuating another mechanism of the device, such as, for example, paddles having an adjustable width. When rotation is no longer required to actuate the mechanism of the device, the retention member 11010 can be rotated while holding the distal gripper 11020—and thereby the proximal head 11030—stationary so that the threaded end 11012 of the retention member 11010 disengages from the threaded opening 11034 of the proximal head 11030. The retention member 11010 and distal gripper 11020 can then be disengaged and withdrawn from the device with a simple proximal pull force applied thereto, as can be seen in FIGS. 421-424. If desired, until the retention member 11010 and distal gripper 11020 are entirely removed from the device, the distal gripper 11020 and retention member 11010 can re-engage the proximal head 11030 with a distal application of force and rotation thereof.

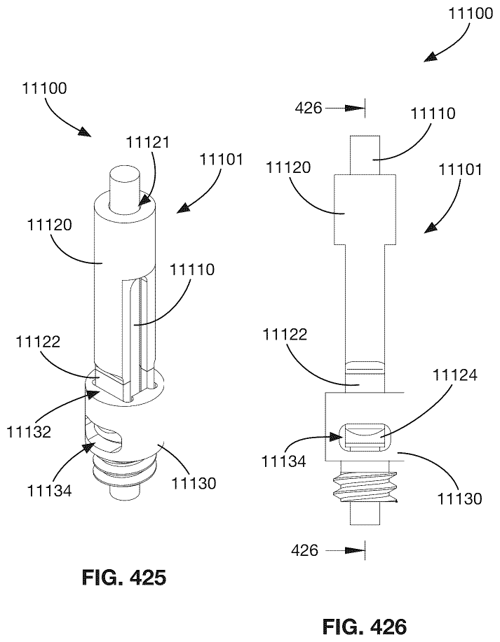

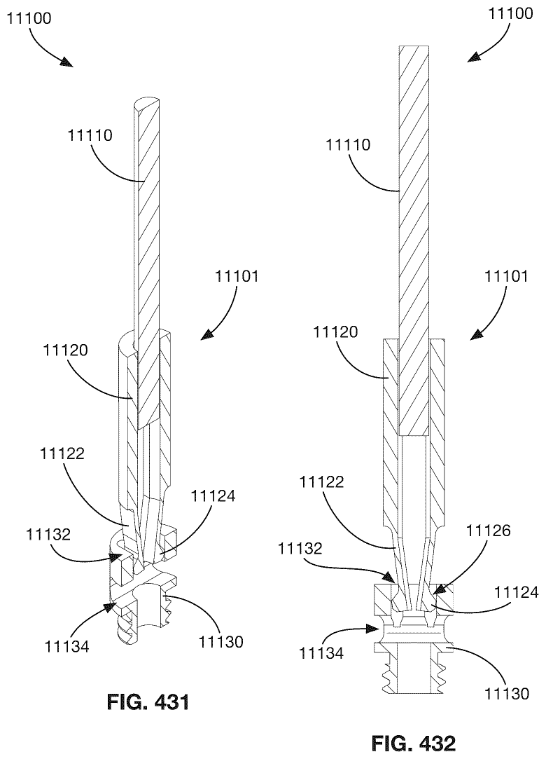

Referring now to FIGS. 425-432, an example coupling assembly 11100 for a device, such as any of the devices disclosed herein, is shown. The coupling assembly 11100 can be used to releasably couple to one or more components of any of the implants or devices disclosed herein. For example, the coupling assembly 11100 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein). In some implementations, an actuation element or member 11101 of the coupling assembly 11100 is moved to open and close the device (e.g., open and close paddled of the device) and a width control element (e.g., width adjustment wire, width adjustment shaft, width adjustment tube, width adjustment line, width adjustment cord, width adjustment suture, etc.), such as the illustrated retention member 11110 is moved to widen and narrow the device (e.g., widen or narrow paddle frames of the device).

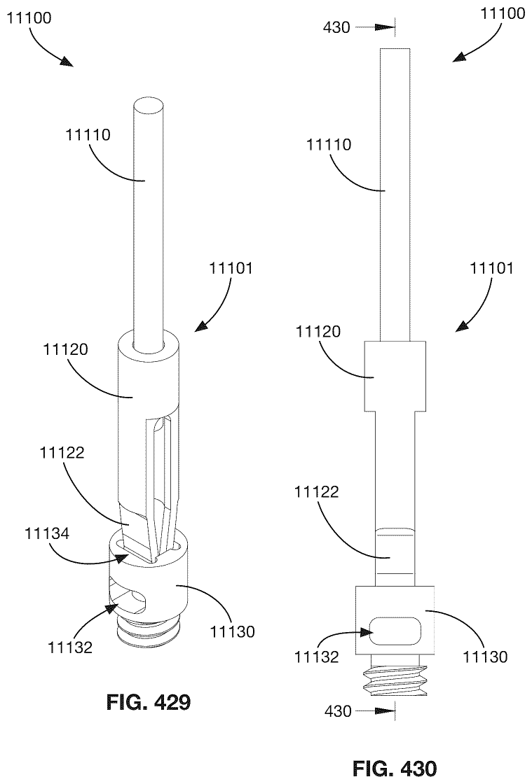

The coupling assembly 11100 is shown in a coupled condition in FIGS. 425-428 and in an uncoupled condition in FIGS. 429-432 as the coupling assembly is initially coupled during implantation of the device and is then uncoupled to disconnect the actuation member 11101 (e.g., tube, shaft, conduit, etc.) from the device. The coupling assembly 11100 includes a retention member 11110 (e.g., wire, rod, tube, shaft, conduit, etc.), a distal gripper 11120 of the actuation member 11101, and a proximal head 11130 of a rotatable actuation member such as, for example, a helical or threaded member like the rotational member 100300 shown in FIGS. 410A-410E and described above. The retention member 11110 can be a pin, a wire (e.g., a nitinol wire), a shaft, or the like.

The distal gripper 11120 includes distally extending flexible fingers 11122 with retention or engaging portions 11124. The flexible fingers 11122 of the distal gripper 11120 are inserted through an opening 11132 in the proximal head 11130 such that the retention or engaging portions 11124 protrude into and engage windows 11134 to securely attach the distal gripper 11120 to the proximal head 11130. Flat surfaces of the flexible fingers 11122 engage corresponding flat surfaces of the opening 11132, thereby facilitating transfer of rotational forces between the distal gripper 11220 and the proximal head 11230. The engaging portions 11124 are sized to fit within the windows 11134 to prevent or inhibit unintended disengagement of distal gripper 11120 from the proximal head 11130. The flexible fingers 11122 and engagement portions 11124 can take on a wide variety of forms suitable for transmission of rotational forces between the distal gripper 11120 and proximal head 11130, such as, for example, fingers or protrusions having a different shape or number.

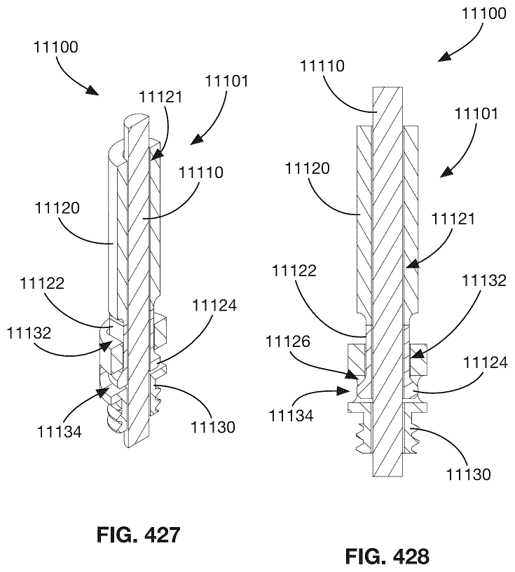

The flexible fingers 11122 can be biased in an inward direction to facilitate engagement and disengagement of the retention or engaging portions 11124 with the windows 11134 of the proximal head 11130. The flexible fingers 11122 and engaging portions 11124 are caused to engage the windows 11134 by inserting the retention member 11110 through a central bore 11121 of the distal gripper 11120 until the retention member 11110 pushes the flexible fingers 11122 outwards so that the engaging portions 11124 project into the windows 11134. The retention member 11110 can extend up to and beyond the length of the distal gripper 11120 (see, e.g., FIGS. 427-428) and can interact with other mechanisms of the device.

During implantation of the device, the distal gripper 11120 of the rotatable actuation member can be rotated and/or oved advanced or retracted to rotate and/or advance or retract the proximal head 11130, thereby actuating another mechanism of the device, such as, for example, paddles having an adjustable width. In some implementations, the retention member 11110 can be advanced/retracted and/or rotated to actuate one mechanism of the device and the distal gripper 11120 can be advanced/retracted and/or rotated to actuate another mechanism of the device. For example, the retention member 11110 can be advanced/retracted and/or rotated to adjust a paddle width of the device and the distal gripper 11120 can be advanced/retracted and/or rotated to open and close paddles of the device (or vice versa).

When rotation is no longer required to actuate the mechanism of the device, the retention member 11110 can be withdrawn proximally from the central bore 11121 of the distal gripper 11120 so that the flexible fingers 11122 of the distal gripper 11120 move inwards and the engaging portions 11124 disengage from the windows 11134 of the proximal head 11130. The retention member 11110 and distal gripper 11120 can then be disengaged and withdrawn from the device with a simple proximal pull force applied thereto, as can be seen in FIGS. 429-432. The engaging portions 11124 can also include a sloped proximal side 11126 to facilitate disengagement of the engaging portions 11124 from the windows 11134 as the distal gripper 11120 is removed from the proximal head 11130. If desired, until the retention member 11110 and distal gripper 11120 are entirely removed from the device, the distal gripper 11120 and retention member 11110 can re-engage the proximal head 11130 with a distal application of force.

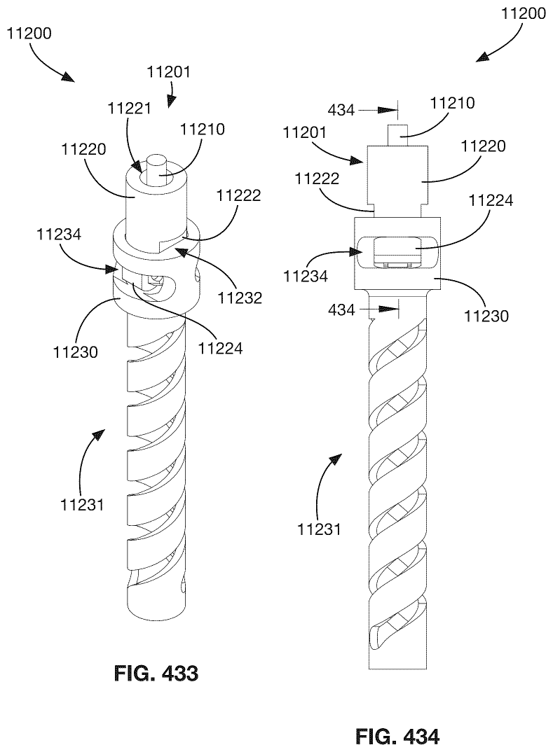

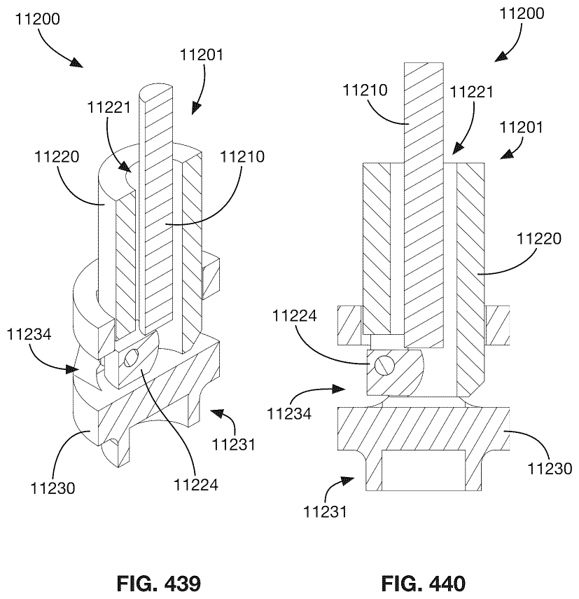

Referring now to FIGS. 433-440, an example coupling assembly 11200 for a device, such as any of the devices disclosed herein, is shown. The coupling assembly 11200 can be used to releasably couple to one or more components of any of the implants or devices disclosed herein. For example, the coupling assembly 11200 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein).

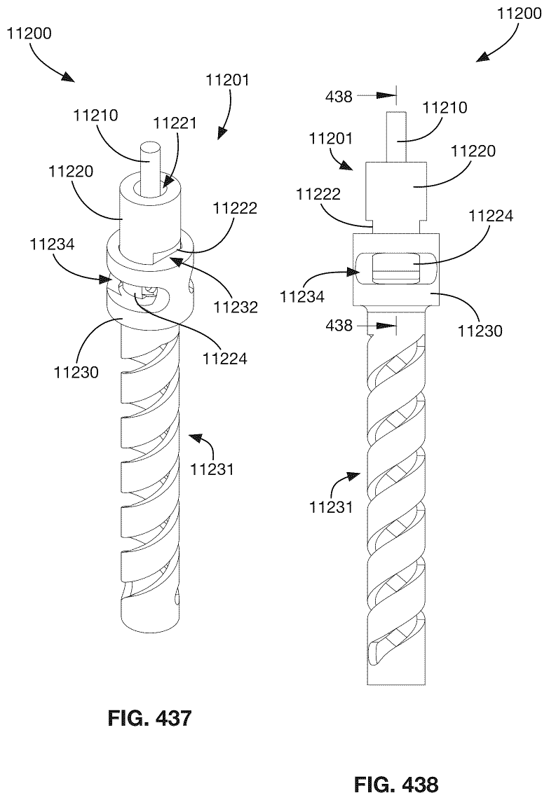

The coupling assembly 11200 is shown in a coupled condition in FIGS. 433-436 and in an uncoupled condition in FIGS. 437-440 as the coupling assembly is initially coupled during implantation of the device and is then uncoupled to disconnect an actuation member 11201 (e.g., tube, shaft, conduit, etc.) from the device. The coupling assembly 11200 includes a width adjustment element (e.g., width adjustment wire, width adjustment shaft, width adjustment tube, width adjustment line, width adjustment cord, width adjustment suture, etc.), such as the illustrated retention member 11210 (e.g., wire, rod, tube, shaft, conduit, etc.), a distal gripper 11220 of the actuation member 11201, and a proximal head 11230 of a coupler, such as the illustrated rotatable actuation member 11231 such as, for example, a helical or threaded member like the rotational member 100300 shown in FIGS. 410A-410E and described above. The retention member 11210 can be a pin, a wire (e.g., a nitinol wire), a shaft, or the like.

The distal gripper 11220 includes a keyed end 11222 and a pivoting engaging member 11224 (e.g., pawl, etc.). The keyed end 11222 is inserted through an opening 11232 in the proximal head 11230 such that the flat surfaces of the keyed end 11222 engage corresponding flat surfaces of the opening 11232, thereby facilitating transfer of rotational forces between the distal gripper 11220 and the proximal head 11230. A pivoting engaging member 11224 protrudes into and engages a window 11234 of the proximal head 11230 to securely attach the distal gripper 11220 to the proximal head 11230. The pivoting engaging portion 11224 is sized to fit within the window 11234 to prevent or inhibit unintended disengagement of distal gripper 11220 from the proximal head 11230. The keyed end 11222 and engaging portion 11224 can take on a wide variety of forms suitable for transmission of rotational forces between the distal gripper 11220 and proximal head 11230, such as, for example, the keyed end having a different shape or there being a different number of engaging portions.

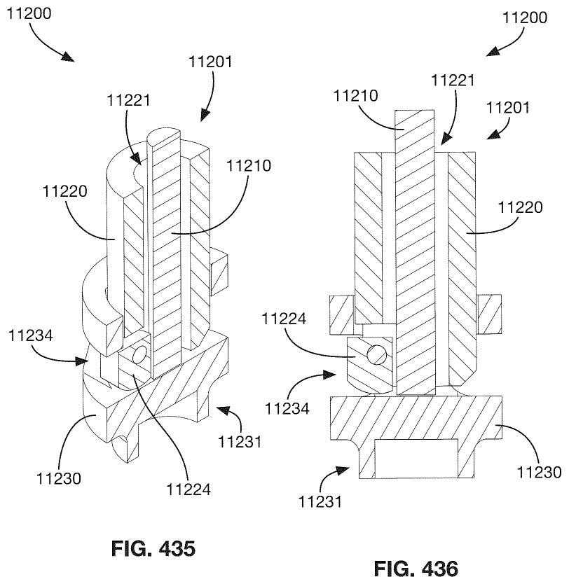

The pivoting engaging member 11224 can be biased—for example, with a spring member—in an inward direction to facilitate engagement and disengagement of the pivoting engaging member 11224 with the window 11234 of the proximal head 11230. The pivoting engaging member 11224 is caused to engage the window 11234 by inserting the retention member 11210 through a central bore 11221 of the distal gripper 11220 until the retention member 11210 pivots the pivoting engaging member 11224 into the window 11234. The retention member 11210 (e.g., wire, rod, tube, shaft, conduit, etc.) can extend up to and beyond the length of the distal gripper 11220 (see, e.g., FIGS. 435-436) and can interact with other mechanisms of the device.

During implantation of the device, the distal gripper 11220 of the rotatable actuation member can be rotated to simultaneously rotate the proximal head 11230, thereby actuating another mechanism of the device, such as, for example, paddles having an adjustable width.

When rotation is no longer required to actuate the mechanism of the device, the retention member 11210 can be withdrawn proximally from the central bore 11221 of the distal gripper 11220 so that the pivoting engaging member 11224 of the distal gripper 11220 moves inwards and disengages from the windows 11234 of the proximal head 11230. The pivoting engaging member 11224 can be moved inward by way of a biasing spring or by withdrawing the distal gripper 11220 in the proximal direction so that the edge of the window 11234 causes the pivoting engaging member 11224 to pivot. The retention member 11210 and distal gripper 11220 can then be disengaged and withdrawn from the device with a simple proximal pull force applied thereto, as can be seen in FIGS. 437-440. If desired, until the retention member 11210 and distal gripper 11220 are entirely removed from the device, the distal gripper 11220 and retention member 11210 can re-engage the proximal head 11230 with a distal application of force.

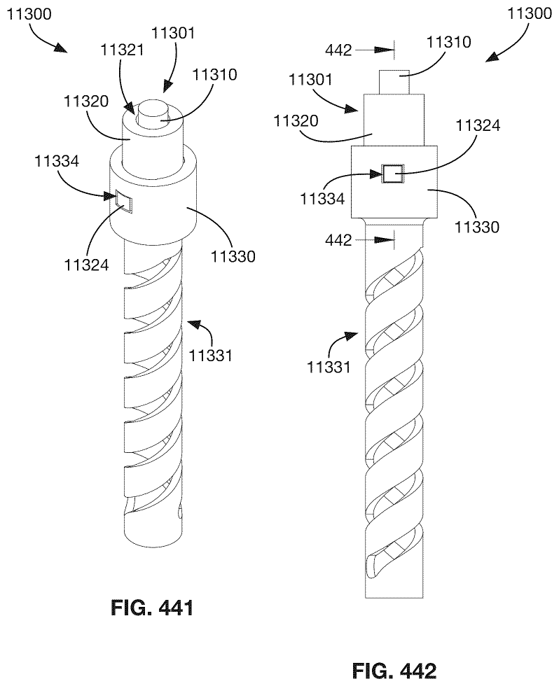

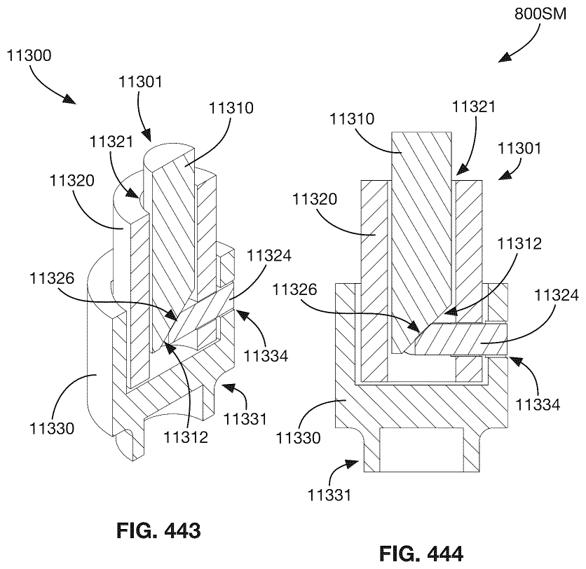

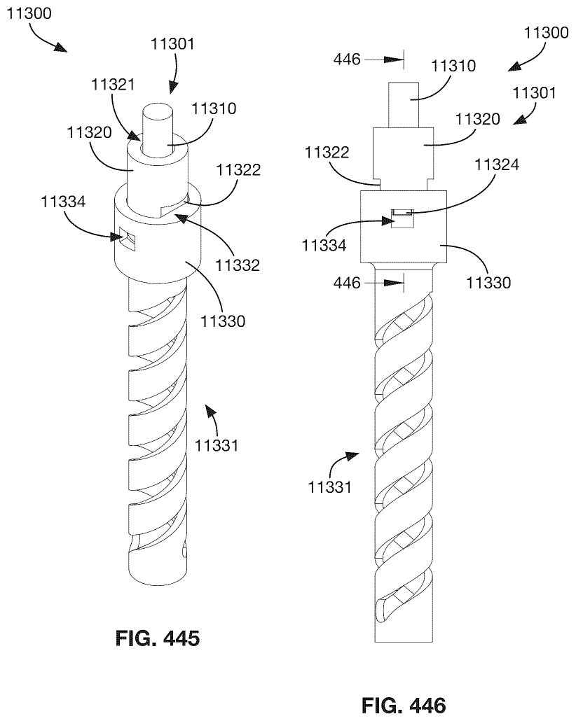

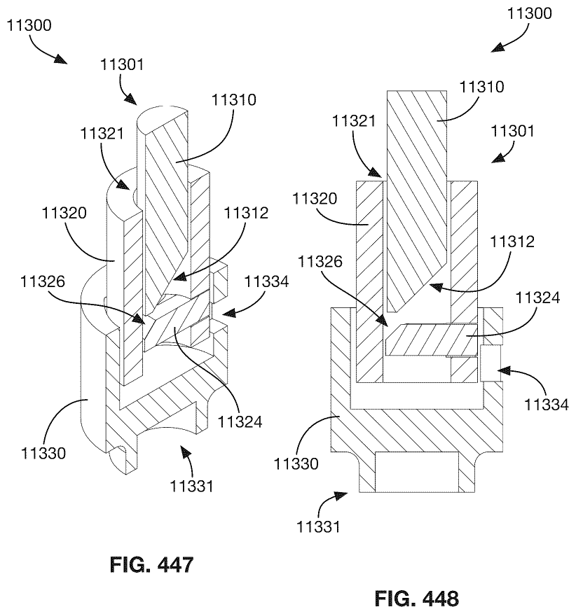

Referring now to FIGS. 441-448, an example coupling assembly 11300 for a device, such as any of the devices disclosed herein, is shown. The coupling assembly 11300 can be used to releasably couple to one or more components of any of the implants or devices disclosed herein. For example, the coupling assembly 11300 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein.

The coupling assembly 11300 is shown in a coupled condition in FIGS. 441-444 and in an uncoupled condition in FIGS. 445-448 as the coupling assembly is initially coupled during implantation of the device and is then uncoupled to disconnect an actuation member 11301 (e.g., tube, shaft, conduit, etc.) from the device. The coupling assembly 11300 includes a width adjustment element (e.g., width adjustment wire, width adjustment shaft, width adjustment tube, width adjustment line, width adjustment cord, width adjustment suture, etc.), such as the illustrated retention member 11310, a distal gripper 11320 of the actuation member 11301, and a proximal head 11330 of a coupler, such as the illustrated rotatable actuation member 11331 such as, for example, a helical or threaded member like the rotational member 100300 shown in FIGS. 410A-410E and described above. The retention member 11310 can be a pin, a wire (e.g., a nitinol wire), a shaft, or the like.

The distal gripper 11320 includes a keyed end 11322 and a sliding engaging member 11324 (e.g., sliding bar, sliding shaft, etc.). The keyed end 11322 is inserted through an opening 11332 in the proximal head 11330 such that the flat surfaces of the keyed end 11322 engage corresponding flat surfaces of the opening 11332, thereby facilitating transfer of rotational forces between the distal gripper 11320 and the proximal head 11330. A sliding engaging member 11324 protrudes into and engages a window 11334 of the proximal head 11330 to securely attach the distal gripper 11320 to the proximal head 11330. The sliding engaging portion 11324 is sized to fit within the window 11334 to prevent or inhibit unintended disengagement of distal gripper 11320 from the proximal head 11330. The keyed end 11322 and engaging portion 11324 can take on a wide variety of forms suitable for transmission of rotational forces between the distal gripper 11320 and proximal head 11330, such as, for example, the keyed end and/or engaging portion having a different shape or number of flat surfaces.

The sliding engaging member 11324 can be biased—for example, with a spring member—in an inward direction to facilitate engagement and disengagement of the sliding engaging member 11324 with the window 11334 of the proximal head 11330. The sliding engaging member 11324 is caused to engage the window 11334 by inserting the retention member 11310 through a central bore 11321 of the distal gripper 11320. The retention member 11310 includes a slanted or beveled surface 11312 that engages a slanted or beveled surfaces 11326 of the sliding retention member 11310 so that the sliding engaging member 11324 moves outward and into the window 11334.

During implantation of the device, the distal gripper 11320 of the can be rotated and/or advanced or retracted to rotate and/or advance or retract the proximal head 11330, thereby actuating another mechanism of the device, such as, for example, paddles having an adjustable width. When rotation is no longer required to actuate the mechanism of the device, the retention member 11310 can be withdrawn proximally from the central bore 11321 of the distal gripper 11320 so that the sliding engaging member 11324 of the distal gripper 11320 moves inwards and disengages from the windows 11334 of the proximal head 11330. The sliding engaging member 11324 can be moved inward by way of a biasing spring (not shown) or, in some implementations, the sliding engaging member 11324 can include a sloped outer portion that engages an edge of the window 11334 to cause the sliding engaging member 11324 to slide radially inwards as the distal gripper 11320 is pulled in a proximal direction. The retention member 11310 and distal gripper 11320 can then be disengaged and withdrawn from the device with a simple proximal pull force applied thereto, as can be seen in FIGS. 445-448. If desired, until the retention member 11310 and distal gripper 11320 are entirely removed from the device, the distal gripper 11320 and retention member 11310 can re-engage the proximal head 11330 with a distal application of force.

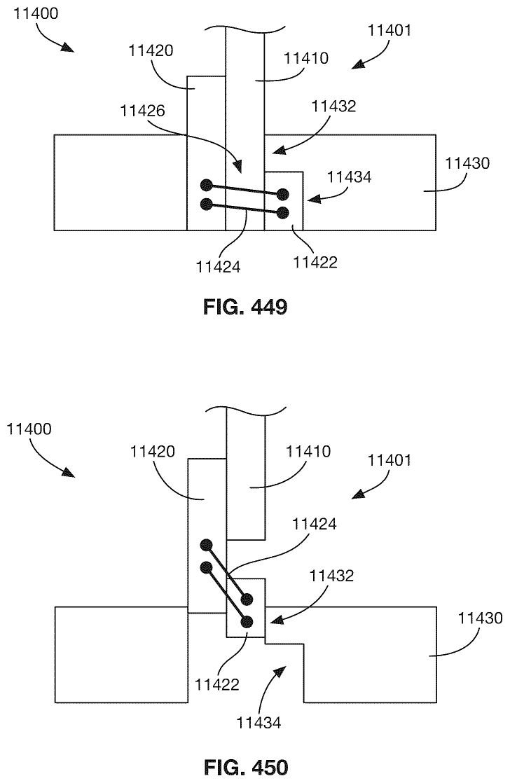

Referring now to FIGS. 449-450, an example coupling assembly 11400 for a device, such as any of the devices disclosed herein, is shown. The coupling assembly 11400 can be used to releasably couple to one or more components of any of the implants or devices disclosed herein. For example, the coupling assembly 11400 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein.

The coupling assembly 11400 is shown in a coupled condition in FIG. 449 and in an uncoupled condition in FIG. 450 as the coupling assembly is initially coupled during implantation of the device and is then uncoupled to disconnect an actuation member 11401 from the device. The coupling assembly 11400 includes a retention member 11410, a distal gripper 11420 of the actuation member 11401, and a proximal head 11430 of a rotatable actuation member such as, for example, a helical or threaded member like the rotational member 100300 shown in FIGS. 410A-410E and described above. The retention member 11410 can be a pin, a wire (e.g., a nitinol wire), a shaft, or the like.

The distal gripper 11420 is inserted through an opening 11432 of the proximal head 11430 to engage the proximal head 11430. The distal gripper 11420 has a non-circular shape that engages the opening 11432 that has a similar shape, thereby facilitating transfer of rotational forces between the distal gripper 11420 and the proximal head 11430. The distal gripper 11420 includes a deployable engaging member 11422 (e.g., bar, block, etc.) that is moveable radially outward to engage a recess 11434 in the proximal head 11430 to securely attach the distal gripper 11420 to the proximal head 11430. A linkage 11424 attaches the deployable engaging member 11422 to the distal gripper 11420. As the distal gripper 11420 is inserted into the opening 11432 and reaches the bottom of the opening 11432, the deployable engaging member 11422 engages the proximal head 11430 and is moved radially outward by way of the linkage 11424 (e.g., a four-bar linkage, etc.) and securely holds the deployable engaging member 11422 within the recess 11434. The deployable engaging member 11422 is sized to fit within the recess 11434 to prevent or inhibit unintended disengagement of distal gripper 11420 from the proximal head 11430. The deployable engaging member 11422 and recess 11434 can take on a wide variety of forms suitable for transmission of rotational forces between the distal gripper 11420 and proximal head 11430, such as, for example, the deployable engaging member and recess can have a different shape.

The deployable engaging member 11422 can be biased—for example, with a spring member—in an inward direction to facilitate engagement and disengagement of the deployable engaging member 11422 with the recess 11434 of the proximal head 11430. The deployable engaging member 11422 is retained within the recess 11434 by inserting the retention member 11410 into a gap 11426 between the distal gripper 11420 and deployable engaging member 11422. The retention member 11410 can extend up to and beyond the length of the distal gripper 11420 (see, e.g., FIG. 449) and can interact with other mechanisms of the device.

During implantation of the device, the distal gripper 11420 of the can be rotated and/or advanced or retracted to rotate and/or advance or retract the proximal head 11430, thereby actuating another mechanism of the device, such as, for example, paddles having an adjustable width. In some implementations, the retention member 11410 can be advanced/retracted and/or rotated to actuate one mechanism of the device and the distal gripper 11420 can be advanced/retracted and/or rotated to actuate another mechanism of the device. For example, the retention member 11410 can be advanced/retracted and/or rotated to adjust a paddle width of the device and the distal gripper 11420 can be advanced/retracted and/or rotated to open and close paddles of the device (or vice versa).

When rotation is no longer required to actuate the mechanism of the device, the retention member 11410 can be withdrawn proximally from the gap 11426 of the distal gripper 11420 so that the deployable engaging member 11422 of the distal gripper 11420 can move inward to disengage from the recess 11434 of the proximal head 11430. The deployable engaging member 11422 can be moved inward by operation of the linkage 11424 or by way of a biasing spring (not shown). The retention member 11410 and distal gripper 11420 can then be disengaged and withdrawn from the device with a simple proximal pull force applied thereto, as can be seen in FIG. 450. If desired, until the retention member 11410 and distal gripper 11420 are entirely removed from the device, the distal gripper 11420 and retention member 11410 can re-engage the proximal head 11430 with a distal application of force.

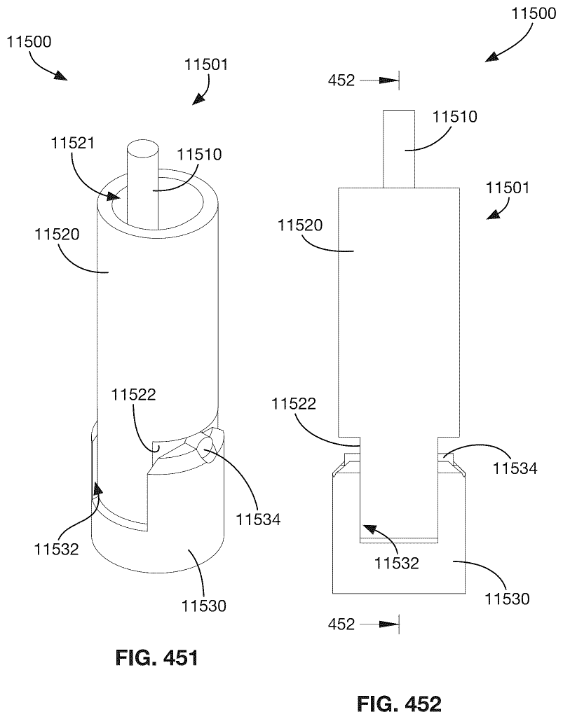

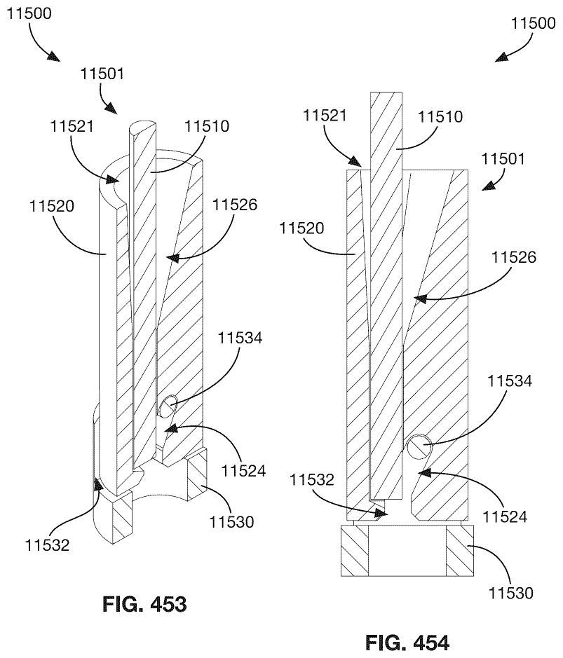

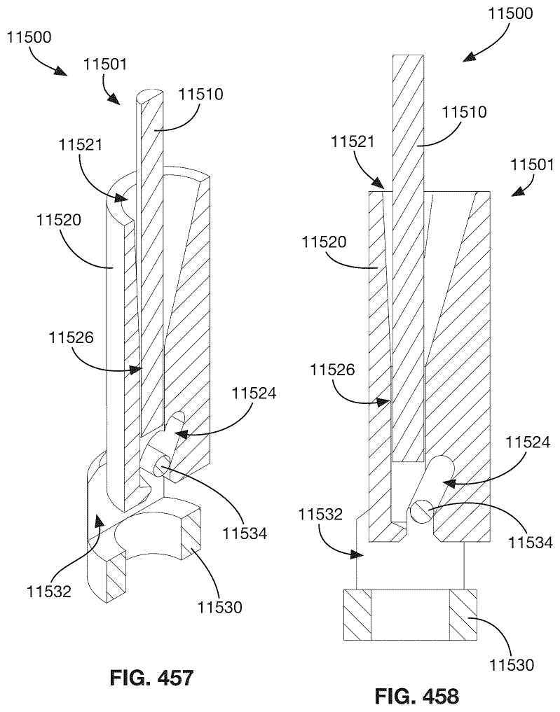

Referring now to FIGS. 451-458, an example coupling assembly 11500 for a device, such as any of the devices disclosed herein, is shown. The coupling assembly 11500 can be used to releasably couple to one or more components of any of the implants or devices disclosed herein. For example, the coupling assembly 11500 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein).

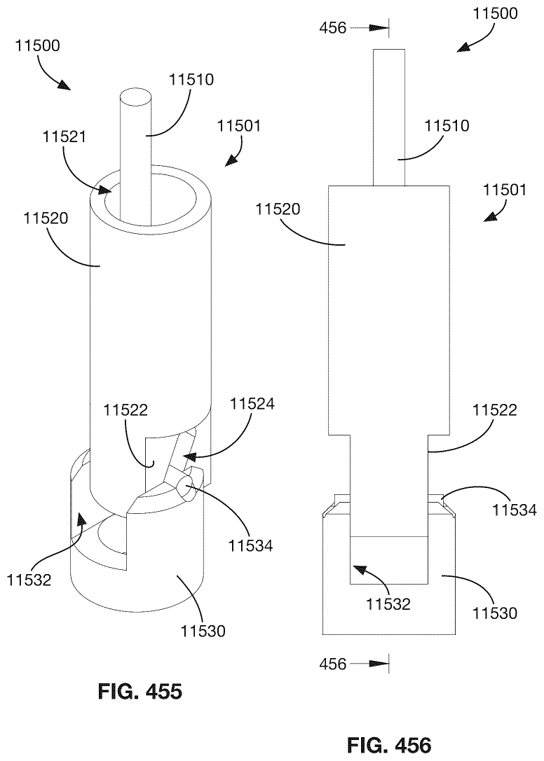

The coupling assembly 11500 is shown in a coupled condition in FIGS. 451-454 and in an uncoupled condition in FIGS. 455-458 as the coupling assembly is initially coupled during implantation of the device and is then uncoupled to disconnect an actuation member 11501 (e.g., tube, shaft, conduit, etc.) from the device. The coupling assembly 11500 includes a retention member 11510, a distal gripper 11520 of the actuation member 11501, and a proximal head 11530 of a rotatable actuation member such as, for example, a helical or threaded member like the rotational member 100300 shown in FIGS. 410A-410E and described above. The retention member 11510 can be a pin, a wire (e.g., a nitinol wire), a shaft, or the like.

The distal gripper 11520 includes a keyed end 11522 and a slot 11524 for receiving a pin 11534 of the proximal head 11530. The keyed end 11522 is inserted through an opening 11532 in the proximal head 11530 such that the flat surfaces of the keyed end 11522 engage corresponding flat surfaces of the opening 11532, thereby facilitating transfer of rotational forces between the distal gripper 11520 and the proximal head 11530. The pin 11534 extends across the opening 11532 between the flat faces of the opening 11532 and has a diameter that is sized to closely fit within the slot 11524. The positions of the slot 11524 and pin 11534 are arranged so that the distal gripper 11520 and the proximal head 11530 are approximately axially aligned when the pin 11534 is fully engaged in the slot 11524. The keyed end 11522 and slot 11524 can take on a wide variety of forms suitable for transmission of rotational forces between the distal gripper 11520 and proximal head 11330, such as, for example, the keyed end and/or engaging portion having a different shape or number of flat surfaces.

The pin 11534 engages and slides into the slot 11524 as the distal gripper 11520 is extended distally to meet and engage the proximal head 11530. The retention member 11510 (e.g., wire, rod, tube, shaft, conduit, etc.) is inserted into a central bore 11521 of the distal gripper 11520 where an optional tapered portion 11526 guides the retention member 11510 to intersect the slot 11524 so that the pin 11534 is prohibited from sliding out of the slot 11524. In some implementations, the retention member 11510 can extend up to and beyond the length of the distal gripper 11520 and can interact with other mechanisms of the device.

During implantation of the device, the distal gripper 11520 of the rotatable actuation member can be rotated and/or advanced/retracted to simultaneously rotate and/or advance/retract the proximal head 11530, thereby actuating another mechanism of the device, such as, for example, paddles having an adjustable width. When rotation is no longer required to actuate the mechanism of the device, the retention member 11510 can be withdrawn proximally from the central bore 11521 of the distal gripper 11520 so that the pin 11534 of the proximal head can slide out of and disengage from the slot 11524 of the distal gripper 11520 as the distal gripper 11520 is moved proximally. The retention member 11510 and distal gripper 11520 can then be disengaged and withdrawn from the device with a simple proximal pull force applied thereto, as can be seen in FIGS. 455-458. If desired, until the retention member 11510 and distal gripper 11520 are entirely removed from the device, the distal gripper 11520 and retention member 11510 can re-engage the proximal head 11530 with a distal application of force.

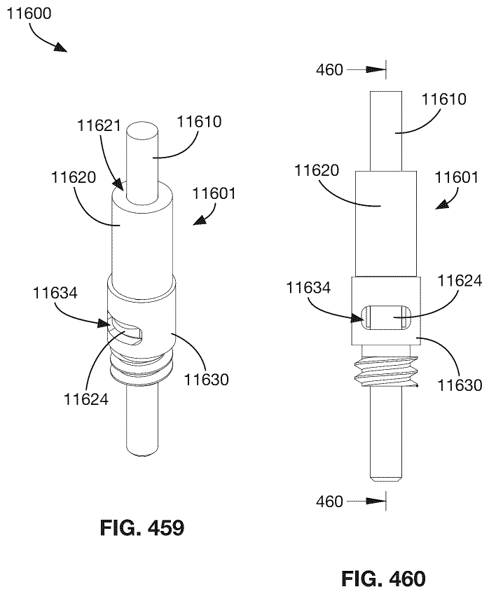

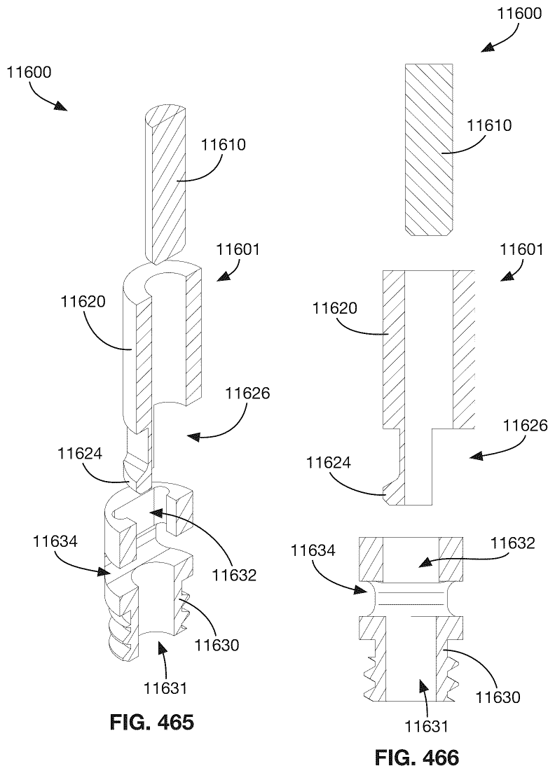

Referring now to FIGS. 459-466, an example coupling assembly 11600 for a device, such as any of the devices disclosed herein, is shown. The coupling assembly 11600 can be used to releasably couple to one or more components of any of the implants or devices disclosed herein. For example, the coupling assembly 11600 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein). In some implementations, an actuation element or member 11601 (e.g., tube, shaft, conduit, etc.) of the coupling assembly 11600 is moved to open and close the device (e.g., open and close paddled of the device) and a width control element, such as the illustrated retention member 11610 is moved to widen and narrow the device (e.g., widen or narrow paddle frames of the device).

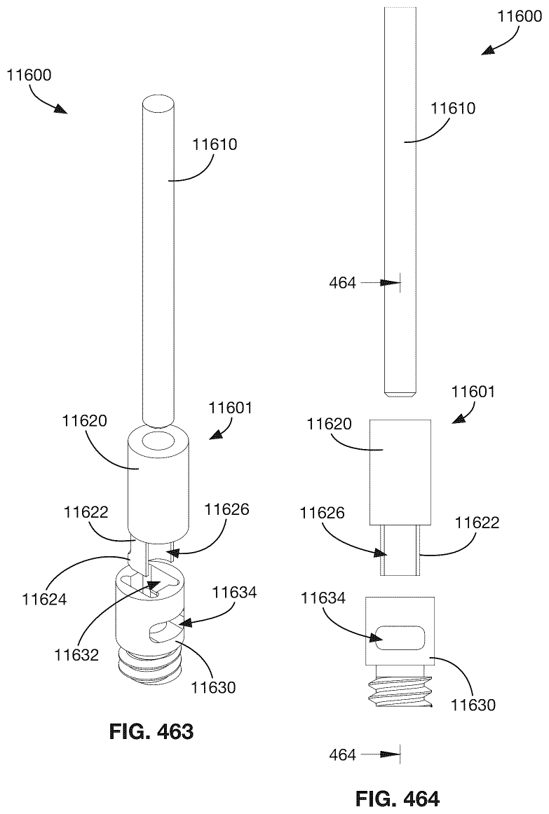

The coupling assembly 11600 is shown in a coupled condition in FIGS. 459-462 and in an uncoupled condition in FIGS. 463-466 as the coupling assembly is initially coupled during implantation of the device and is then uncoupled to disconnect an actuation member 11601 (e.g., wire, rod, tube, shaft, conduit, etc.) from the device. The coupling assembly 11600 includes a retention member 11610, a distal gripper 11620 of the actuation member 11601, and a proximal head 11630 of a rotatable actuation member such as, for example, a helical or threaded member like the rotational member 100300 shown in FIGS. 410A-410E and described above. The retention member 11610 can be a pin, a wire (e.g., a nitinol wire), a shaft, or the like.

The distal gripper 11620 includes a keyed end 11622 that is inserted through an opening 11632 in the proximal head 11630 such that the flat surfaces of the keyed end 11622 engage corresponding flat surfaces of the opening 11632, thereby facilitating transfer of rotational forces between the distal gripper 11620 and the proximal head 11630. The keyed end 11622 includes an engaging portion 11624 that is sized to fit within a window 11634 of the proximal head 11630 to prevent or inhibit unintended disengagement of distal gripper 11620 from the proximal head 11630. The keyed end 11622 and opening 11632 can take on a wide variety of forms suitable for transmission of rotational forces between the distal gripper 11620 and proximal head 11630, such as, for example, by having a different shape.

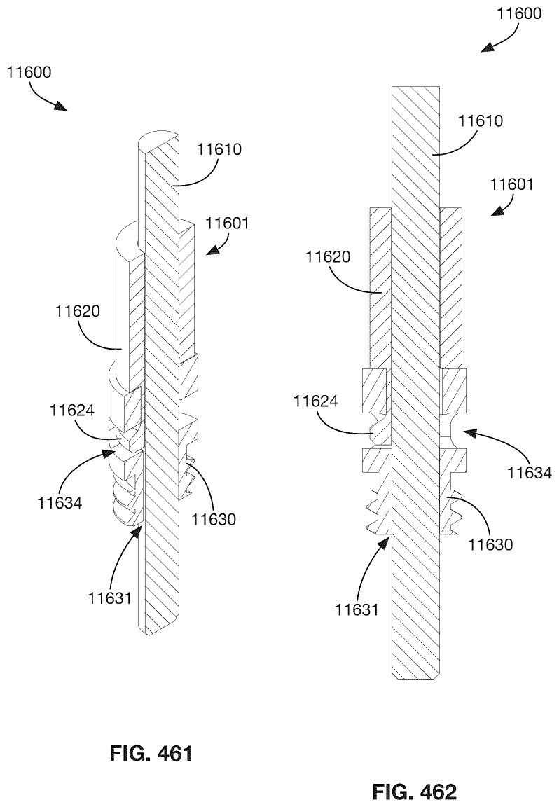

The keyed end 11622 is sized to fit within the opening 11632 only when the distal gripper 11620 and proximal head 11630 are offset axially. The distal gripper 11620 and proximal head 11630 can be axially aligned when the engaging portion 11624 is aligned with and inserted into the window 11634. Once the engaging portion 11624 is inserted into the window 11634, the retention member 11610 can inserted through a central bore 11621 of the distal gripper 11620 and into a central bore 11631 of the proximal head 11630 to prohibit the engaging member 11624 from disengaging from the window 11634. The retention member 11610 can extend up to and beyond the length of the distal gripper 11620 (see, e.g., FIGS. 461-462) and can interact with other mechanisms of the device.

During implantation of the device, the distal gripper 11620 of the rotatable actuation member can be rotated and/or retracted/advanced to rotate and/or advance/retract the proximal head 11630, thereby actuating another mechanism of the device, such as, for example, paddles having an adjustable width. In some implementations, the retention member 11610 can be advanced/retracted and/or rotated to actuate one mechanism of the device and the distal gripper 11620 can be advanced/retracted and/or rotated to actuate another mechanism of the device. For example, the retention member 11610 can be advanced/retracted and/or rotated to adjust a paddle width of the device and the distal gripper 11620 can be advanced/retracted and/or rotated to open and close paddles of the device (or vice versa).

When rotation is no longer required to actuate the mechanism of the device, the retention member 11610 can be withdrawn proximally from the central bore 11621 of the distal gripper 11620 and the central bore 11631 of the proximal head 11630 so that the engaging member 11624 can disengage from the window 11634. With the retention member 11610 removed, a recess or opening 11626 provides room for the engaging member 11624 of the keyed end 11622 to retract from the window 11634. The retention member 11610 and distal gripper 11620 can then be disengaged and withdrawn from the device with a simple proximal pull force applied thereto, as can be seen in FIGS. 463-466. If desired, until the retention member 11610 and distal gripper 11620 are entirely removed from the device, the distal gripper 11620 and retention member 11610 can re-engage the proximal head 11630 with a distal application of force.

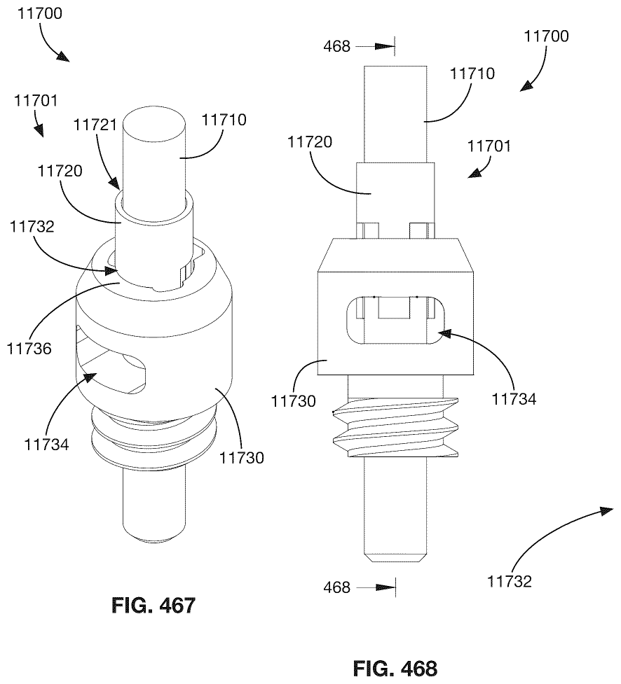

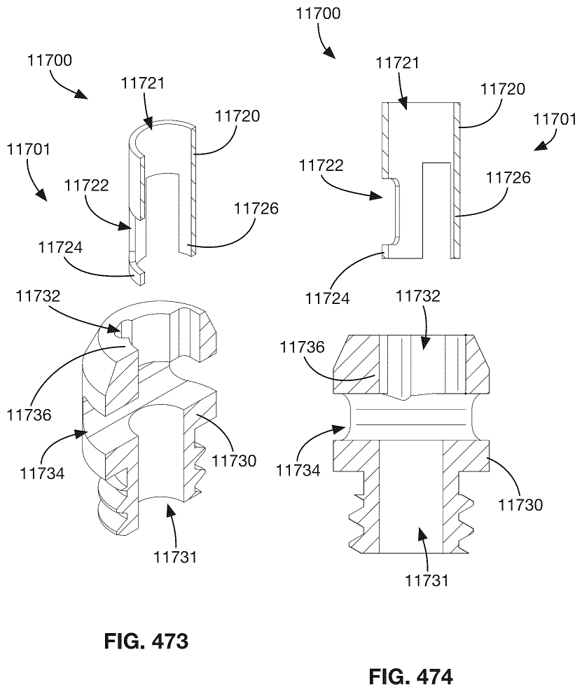

Referring now to FIGS. 467-474, an example coupling assembly 11700 for a device, such as any of the devices disclosed herein, is shown. The coupling assembly 11700 can be used to releasably couple to one or more components of any of the implants or devices disclosed herein. For example, the coupling assembly 11700 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein). In one implementation, an actuation element or member 11701 (e.g., tube, shaft, conduit, etc.) of the coupling assembly 11700 is moved to open and close the device (e.g., open and close paddled of the device) and a width control element, such as the illustrated retention member 11710 (e.g., wire, rod, tube, shaft, conduit, etc.) is moved to widen and narrow the device (e.g., widen or narrow paddle frames of the device).

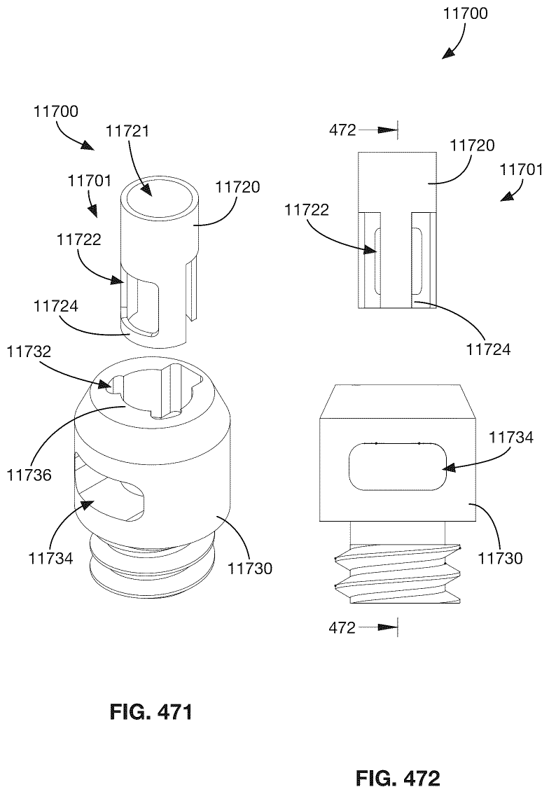

The coupling assembly 11700 is shown in a coupled condition in FIGS. 467-470 and in an uncoupled condition in FIGS. 471-474 as the coupling assembly is initially coupled during implantation of the device and is then uncoupled to disconnect an actuation member 11701 from the device. The coupling assembly 11700 includes a retention member 11710, a distal gripper 11720 of the actuation member 11701, and a proximal head 11730 of a rotatable actuation member such as, for example, a helical or threaded member like the rotational member 100300 shown in FIGS. 410A-410E and described above. The retention member 11710 can be a pin, a wire (e.g., a nitinol wire), a shaft, or the like.

The distal gripper 11720 is inserted into an opening 11732 of the proximal head 11730 and includes a window 11722 for receiving a protrusion 11736 into the opening 11732. The window 11722 and protrusion 11736 are sized and shaped such that the flat surfaces of the window 11722 engage corresponding flat surfaces of the engaging protrusion 11736, thereby facilitating transfer of rotational forces between the distal gripper 11720 and the proximal head 11730. An engaging portion 11724 of the distal gripper 11720 fits into an opening 11734 of the proximal head 11730 to engage a distal side of the protrusion 11736 to prevent or inhibit unintended disengagement of distal gripper 11720 from the proximal head 11730. The window 11722 and engaging protrusion 11736 can take on a wide variety of forms suitable for transmission of rotational forces between the distal gripper 11720 and proximal head 11730, such as, for example, by having a different shape and/or number.

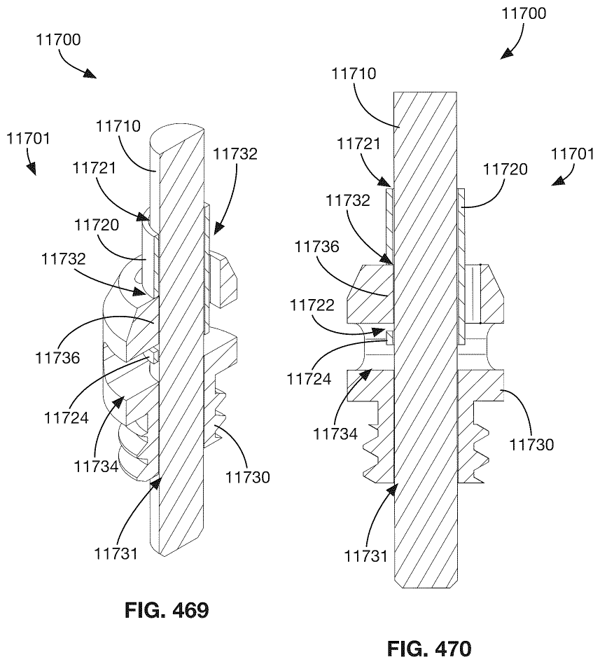

The engaging protrusion 11736 is sized to fit within the window 11722 only when the distal gripper 11720 and proximal head 11730 are offset axially. The distal gripper 11720 and proximal head 11730 can be axially aligned when the engaging protrusion 11736 is aligned with and inserted into the window 11722. Once the engaging protrusion 11736 is inserted into the window 11722, the retention member 11710 can be inserted through a central bore 11721 of the distal gripper 11720 and into a central bore 11731 of the proximal head 11730 to prohibit the engaging protrusion 11736 from disengaging from the window 11722. The retention member 11710 can extend up to and beyond the length of the distal gripper 11720 (see, e.g., FIGS. 469-470) and can interact with other mechanisms of the device.

During implantation of the device, the distal gripper 11720 of the rotatable actuation member can be rotated and/or advanced or retracted to simultaneously rotate and/or advance or retract the proximal head 11730, thereby actuating another mechanism of the device, such as, for example, paddles having an adjustable width. In some implementations, the retention member 11710 can be advanced/retracted and/or rotated to actuate one mechanism of the device and the distal gripper 11720 can be advanced/retracted and/or rotated to actuate another mechanism of the device. For example, the retention member 11710 can be advanced/retracted and/or rotated to adjust a paddle width of the device and the distal gripper 11720 can be advanced/retracted and/or rotated to open and close paddles of the device (or vice versa).

When rotation is no longer required to actuate the mechanism of the device, the retention member 11710 can be withdrawn proximally from the central bore 11721 of the distal gripper 11720 and the central bore 11731 of the proximal head 11730 so that the engaging protrusion 11736 can disengage from the window 11722. With the retention member 11710 removed, a prong or extension 11726 of the distal gripper 11720 can move within the keyed opening 11732 away from the engaging protrusions 11736 as the distal gripper 11720 disengages. The retention member 11710 and distal gripper 11720 can then be disengaged and withdrawn from the device with a simple proximal pull force applied thereto, as can be seen in FIGS. 471-474. If desired, until the retention member 11710 and distal gripper 11720 are entirely removed from the device, the distal gripper 11720 and retention member 11710 can re-engage the proximal head 11730 with a distal application of force.

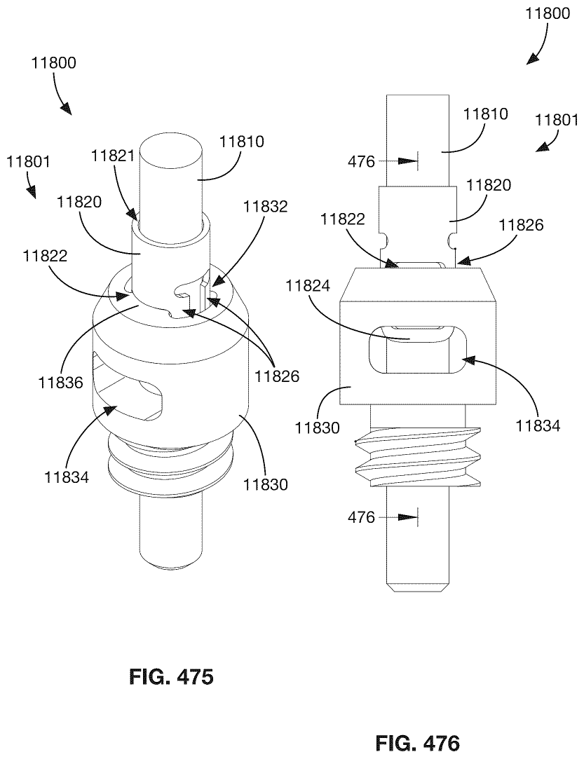

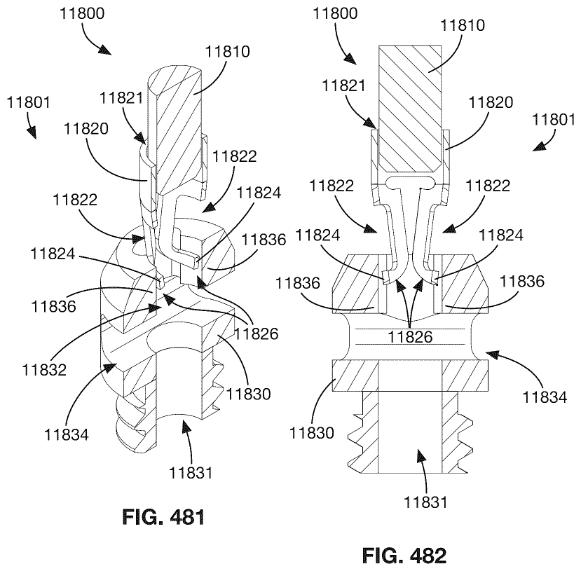

Referring now to FIGS. 475-482, an example coupling assembly 11800 for a device, such as any of the devices disclosed herein, is shown. The coupling assembly 11800 can be used to releasably couple to one or more components of any of the implants or devices disclosed herein. For example, the coupling assembly 11800 can attach to a proximal end of an implant or device (e.g., to hold and position the device), can attach to a component of the implant or device that is moved to open and close the device (e.g., use as an actuation element of any of the devices described herein) and/or can attach to a component of the implant or device that is moved to widen or narrow paddle frames of the device (e.g., use as a width adjustment element of any of the devices described herein). In some implementations, an actuation element or member 11801 (e.g., wire, tube, line, suture, etc.) of the coupling assembly 11800 is moved to open and close the device (e.g., open and close paddles of the device) and a width control element (e.g., wire, tube, line, suture, etc.), such as the illustrated retention member 11810 (e.g., wire, rod, tube, shaft, conduit, etc.) is moved to widen and narrow the device (e.g., widen or narrow paddle frames of the device).

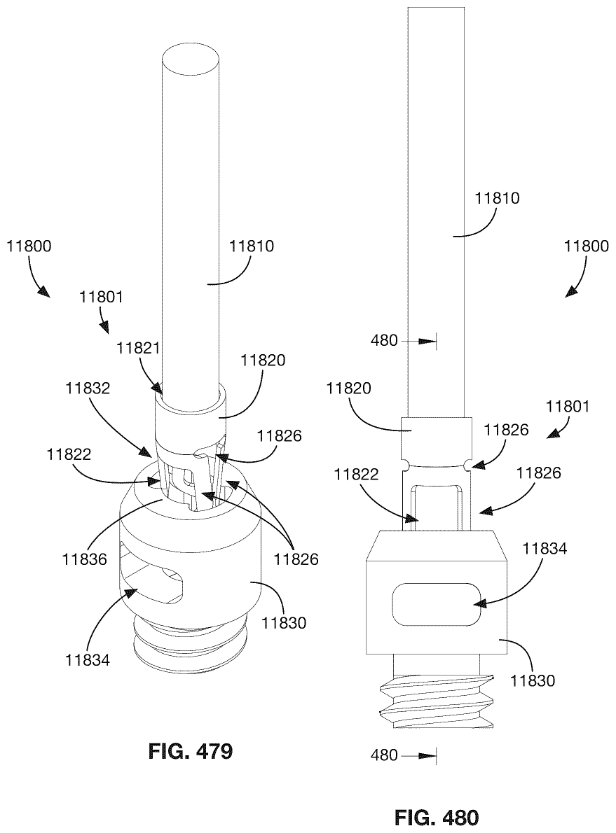

The coupling assembly 11800 is shown in a coupled condition in FIGS. 475-478 and in an uncoupled condition in FIGS. 479-482 as the coupling assembly is initially coupled during implantation of the device and is then uncoupled to disconnect an actuation member 11801 from the device. The coupling assembly 11800 includes a retention member 11810, a distal gripper 11820 of the actuation member 11801, and a proximal head 11830 of a rotatable actuation member 11831 such as, for example, a helical or threaded member like the rotational member 100300 shown in FIGS. 410A-410E and described above. The retention member 11810 can be a pin, a wire (e.g., a nitinol wire), a shaft, or the like.

The distal gripper 11820 includes distally extending flexible fingers 11826 that each have a window 11822 and an engaging portion 11824. The flexible fingers 11826 of the distal gripper 11820 are inserted through an opening 11832 in the proximal head 11830 such that the windows 11822 receive engaging protrusions 11836 of the proximal head 11830. The windows 11822 and the engaging protrusions 11836 are sized and shaped such that the flat surfaces of the window 11822 engage corresponding flat surfaces of the engaging protrusion 11836, thereby facilitating transfer of rotational forces between the distal gripper 11820 and the proximal head 11830. The engaging portions 11824 of the flexible fingers 11826 fit into openings 11834 of the proximal head 11830 to engage distal sides of the engaging protrusions 11836 to prevent or inhibit unintended disengagement of distal gripper 11820 from the proximal head 11830. The windows 11822 and engaging protrusions 11836 can take on a wide variety of forms suitable for transmission of rotational forces between the distal gripper 11820 and proximal head 11830, such as, for example, by having a different shape and/or number.

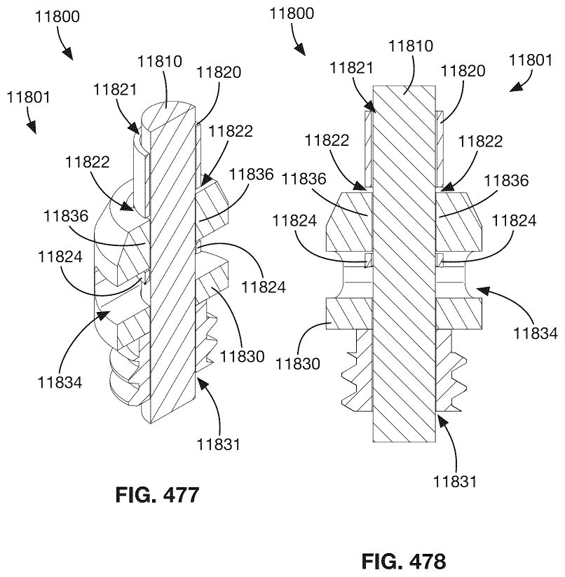

The flexible fingers 11826 can be biased in an inward direction to facilitate engagement and disengagement of the windows 11822 with the engaging protrusions 11836 of the proximal head 11830. The windows 11822 and engaging portions 11824 of the flexible fingers 11826 are caused to engage the engaging portions 11836 of the proximal head 11830 by inserting the retention member 11810 through a central bore 11821 of the distal gripper 11820 until the retention member 11810 pushes the flexible fingers 11826 outwards so that the engaging portions 11836 project into the windows 11822. The retention member 11810 can extend up to and beyond the length of the distal gripper 11820 (see, e.g., FIGS. 477-478) and can interact with other mechanisms of the device.

During implantation of the device, the distal gripper 11820 of the rotatable actuation member can be rotated and/or be advanced/retracted to simultaneously rotate and/or advance/retract the proximal head 11830, thereby actuating another mechanism of the device, such as, for example, paddles having an adjustable width. In some implementations, the retention member 11810 can be advanced/retracted and/or rotated to actuate one mechanism of the device and the distal gripper 11820 can be advanced/retracted and/or rotated to actuate another mechanism of the device. For example, the retention member 11810 can be advanced/retracted and/or rotated to adjust a paddle width of the device and the distal gripper 11820 can be advanced/retracted and/or rotated to open and close paddles of the device (or vice versa).

When rotation is no longer required to actuate the mechanism of the device, the retention member 11810 can be withdrawn proximally from the central bore 11821 of the distal gripper 11820 so that the flexible fingers 11826 of the distal gripper 11820 move inwards and the engaging portions 11836 disengage from the windows 11822 of the proximal head 11830. The retention member 11810 and distal gripper 11820 can then be disengaged and withdrawn from the device with a simple proximal pull force applied thereto, as can be seen in FIGS. 479-482. In some implementations, the flexible fingers 11826 are not biased inward but include sloped portions that engage correspondingly sloped portions of the proximal head 11830 to facilitate inward movement of the flexible fingers during engagement and disengagement of the distal gripper 11820 and the proximal head 11830. If desired, until the retention member 11810 and distal gripper 11820 are entirely removed from the device, the distal gripper 11820 and retention member 11810 can re-engage the proximal head 11830 with a distal application of force.

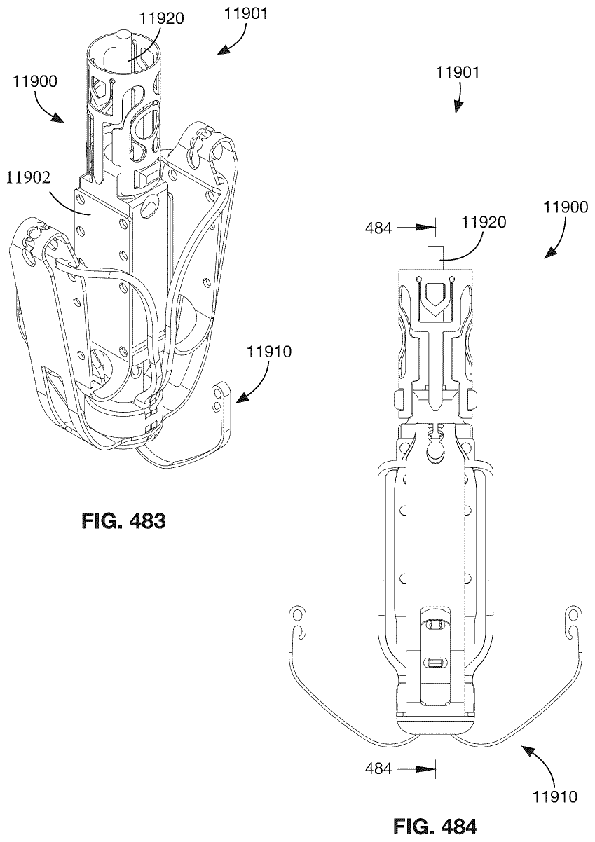

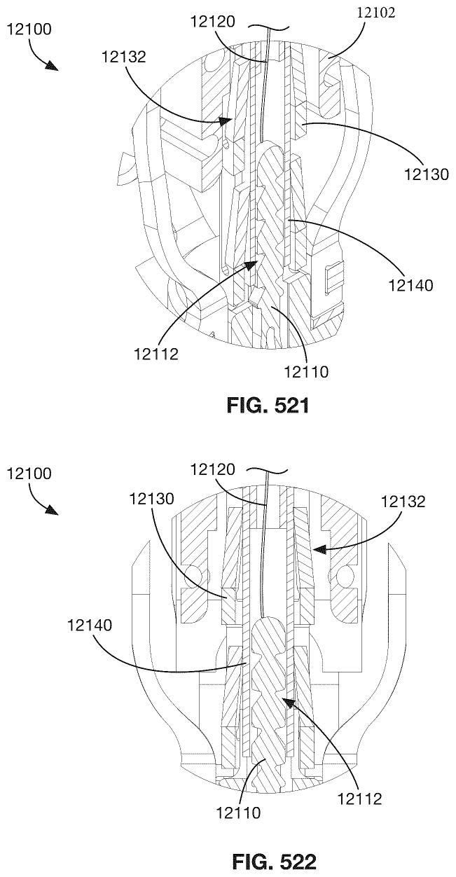

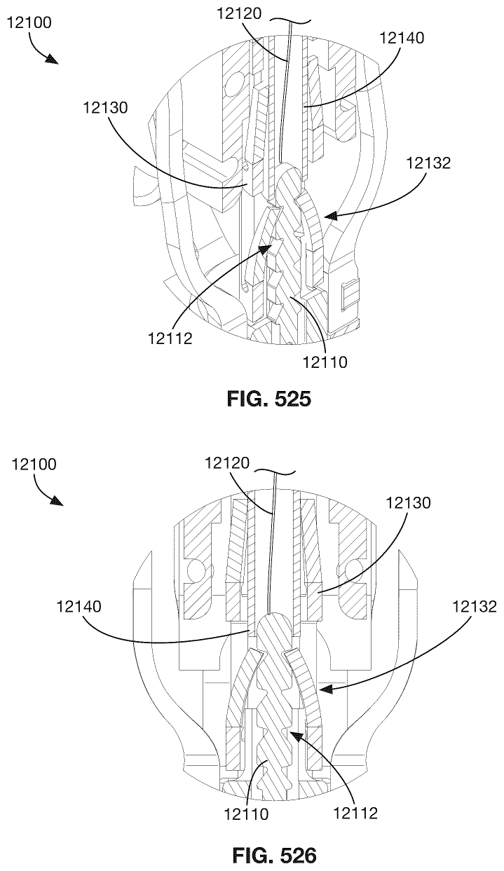

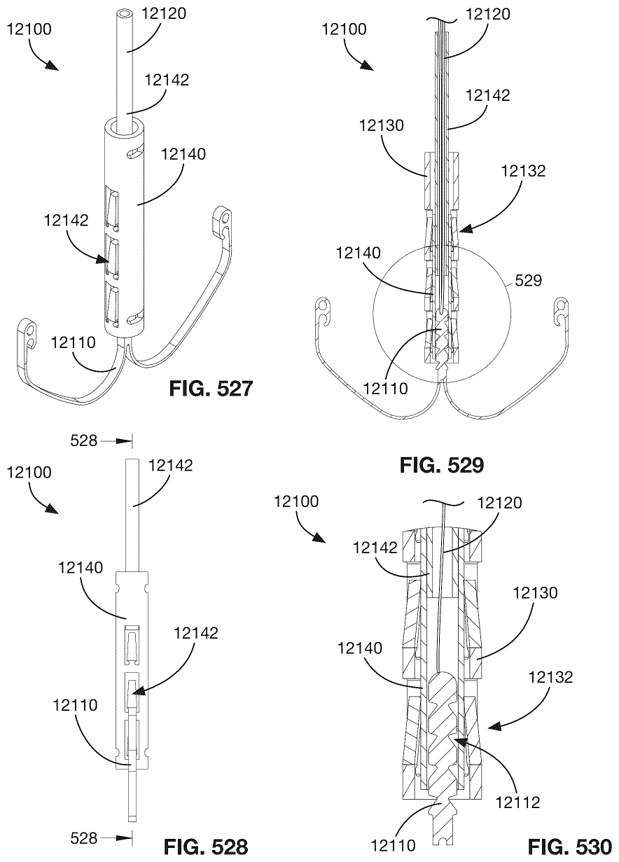

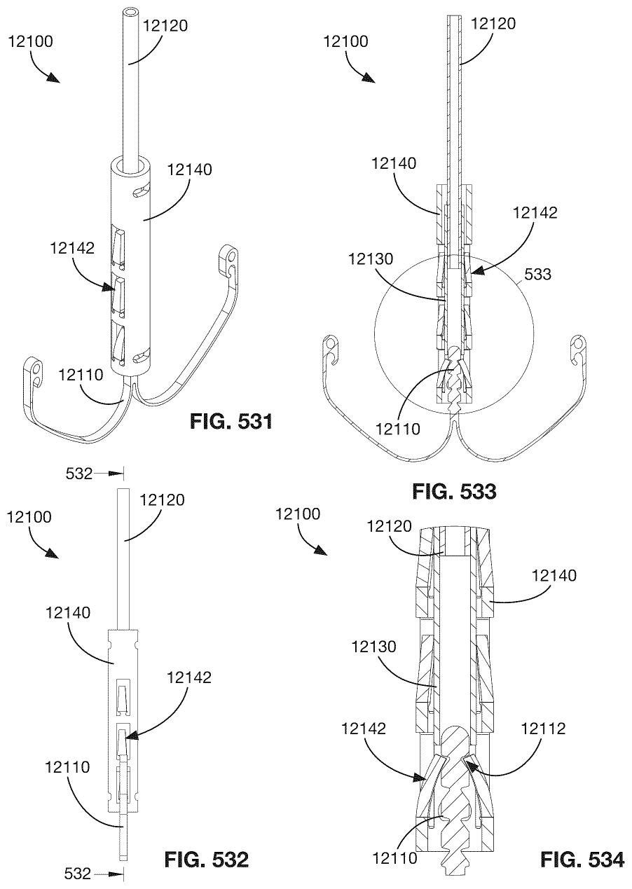

In some implementations of a device, a paddle member can be actuated (e.g., opened, closed, widened, or narrowed) via a linear movement mechanism and locked in place via a locking mechanism. That is, an actuation mechanism—such as those shown in FIGS. 417-482 and described above—can be used to adjust the position and/or size of an adjustable paddle or other member and the locking mechanism can lock the actuation mechanism into one of a plurality of predetermined locations. The locking mechanism can be incorporated into the actuation mechanism such that withdrawal of an actuation member upon completion of actuation causes the locking mechanism to lock the actuated member in place. Some implementations of locking mechanisms that facilitate locking the position of an actuated member with a device are shown in FIGS. 483-534. In each of these implementations, movement and actuation of latching or locking members is accomplished via simplified linear actuations—i.e., pull components—that can be detached and retrieved from the patient's body after actuating a mechanism and locking the mechanism in a desired state.

Referring now to FIGS. 483-497, an actuation mechanism 11900 for a device 11901 is shown. The actuation mechanism 11900 can be used with any of the devices described herein. The device 11901 can be opened and closed in the same manner as any of the implementations disclosed herein. In some implementations, an actuation element (e.g., any of the actuation elements disclosed herein) is coupled to a head 11950 that is threadably or otherwise attached to a receiver 11940 (e.g., an internally threaded element, a notched receiving portion, a column, a lumen, a tube, a shaft, a sleeve, a post, a housing, tracks, a cylinder, etc.). The head 11950 and attached receiver 11940 are advanced and retracted relative to a body or coaptation element 11902 (e.g., spacer, coaption element, gap filler, etc.) of the device to open and close the paddles of the device.

The actuation mechanism 11900 includes an adjustable width connector 11910 (e.g., shaped metal component, shaped plastic component, tether, wire, strut, line, cord, suture, etc.) that is coupled to paddle frames (outer paddle frame portions are not shown in FIGS. 483-486) and that is adjusted by a width adjustment element (e.g., width adjustment wire, width adjustment shaft, width adjustment tube, width adjustment line, width adjustment cord, width adjustment suture, etc.), such as the illustrated pull or actuation member 11920. The actuation member 11920 extends to a distal end that is removably attached to a coupler, such as the illustrated latch tube 11930. The latch tube 11930 is attached to the connector 11910 so that a width of the paddle frames can be adjusted by the actuation member 11920 via the latch tube 11930. The connector 11910 is attached to the latch tube 11930 via a pin 11912 that is received within openings 11936 near the distal end of the latch tube 11930. The connector 11910 can be attached to the latch tube 11930 in any suitable way, such as, for example, via a weld connection, an adhesive, a threaded connection, or the like.

The actuation member 11920 and latch tube 11930 extend coaxially within a receiver 11940 that is threadably or otherwise attached to a proximal head 11950. The receiver 11940 can be attached to other components or mechanisms of the device 11901. The proximal head 11950 can be engaged and actuated in a wide variety of ways described herein (such as, for example, via the mechanisms shown in FIGS. 417-482) to facilitate the implantation of the devices described herein, such as, for example, by opening and closing paddles of the device. The actuation member 11920 can be any suitable material, such as, for example, a nitinol wire.

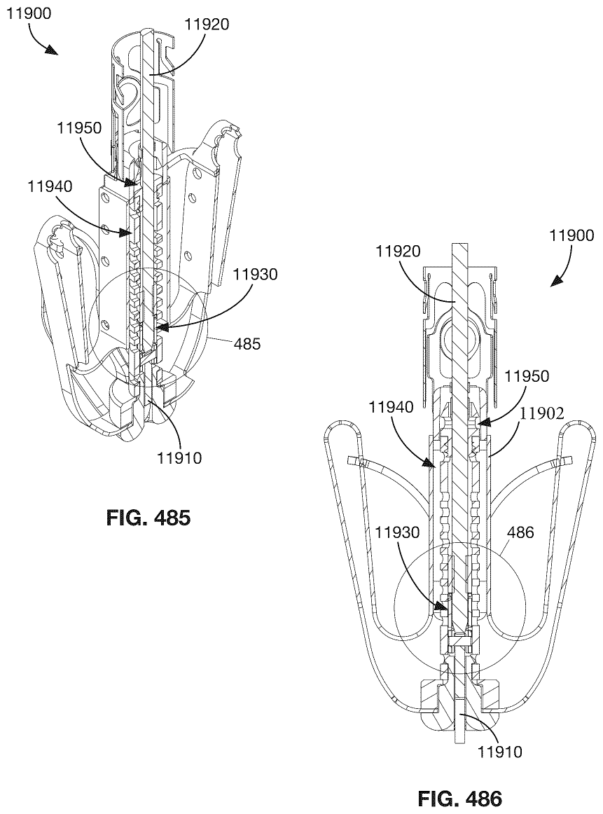

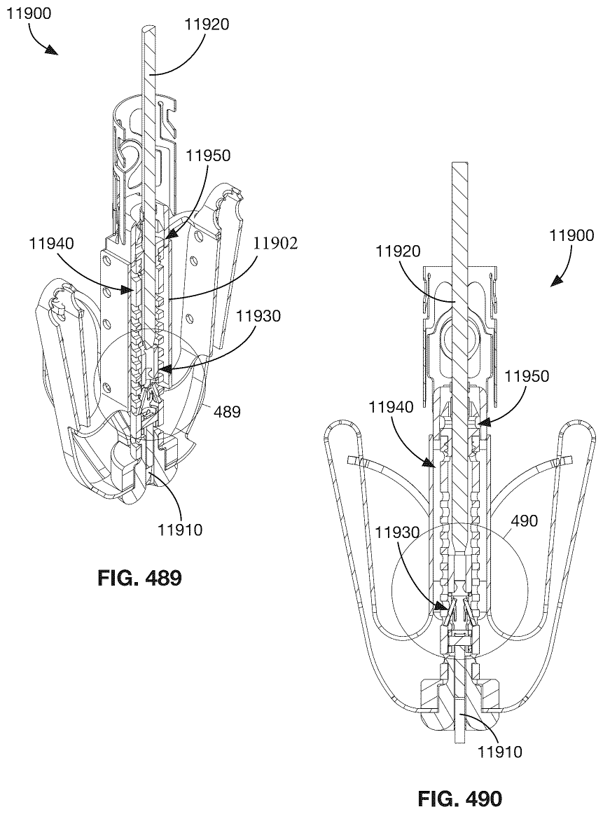

The latch tube 11930 includes two latch members 11932 (e.g., arms, etc.) that can be moved between a latched and an unlatched condition. Referring now to FIGS. 485-488, the latch members 11932 of the latch tube 11930 are shown in an unlatched condition. When the latch members 11932 are in the unlatched condition, the latch tube 11930 can be moved freely along the length of the receiver 11940 to extend and retract the connector 11910. In the latched condition, shown in FIGS. 489-492, the latch members 11932 in the latched condition engage openings 11942 of the receiver 11940 to secure the position of the latch tube 11930 relative to the receiver 11940. Consequently, the position of the connector 11910 relative to the paddles of the device 11901 is not change by actuation of the receiver 11940 and proximal head 11950.

The latch tube 11930 can include any number of latch members 11932 suitable for engaging corresponding openings 11942 in the receiver 11940. The latch members 11932 are integrally formed in the sides of the latch tube 11930 by laser-cutting portions of the latch tube 11930. The latch members 11932 are bent and shape-set in the latched condition so that in a free state the latch members 11932 are angled relative to a central axis of the latch tube 11930. For example, as it shown in FIG. 497, the proximal or upper ends of the latch members 11932 are biased radially inward and the opposite lower or distal ends of the latch members 11932 are biased radially outward. Alternatively, the proximal ends of the latch members 11932 can be biased radially outward and the opposite distal ends of the latch members 11932 can be biased radially inward. In some implementations, the connector 11910 is biased to extend or retract and the latch members 11932 are angled outward to oppose the bias of the connector 11910. For example, in an example where the connector 11910 is biased to move in the proximal direction, the proximal ends of the latch members 11932 can be biased radially outward so that the latch members 11932 engage the openings 11942 of the receiver 11940 such that the proximal movement of the connector 11910 is resisted by the latch members 11932.

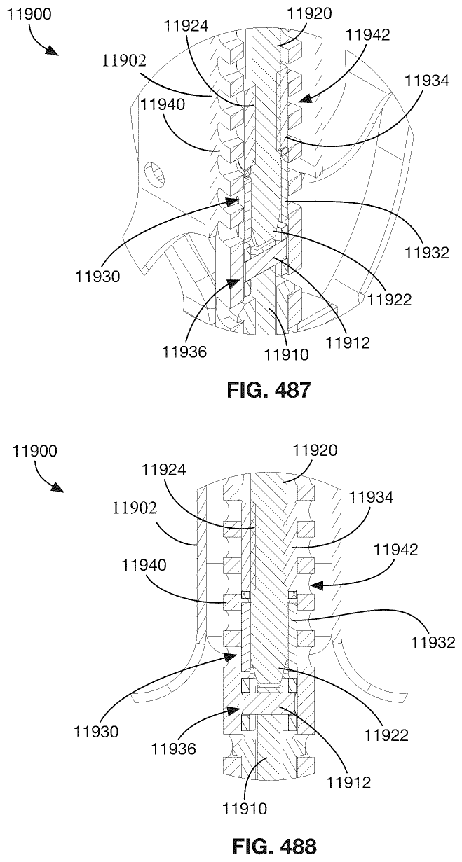

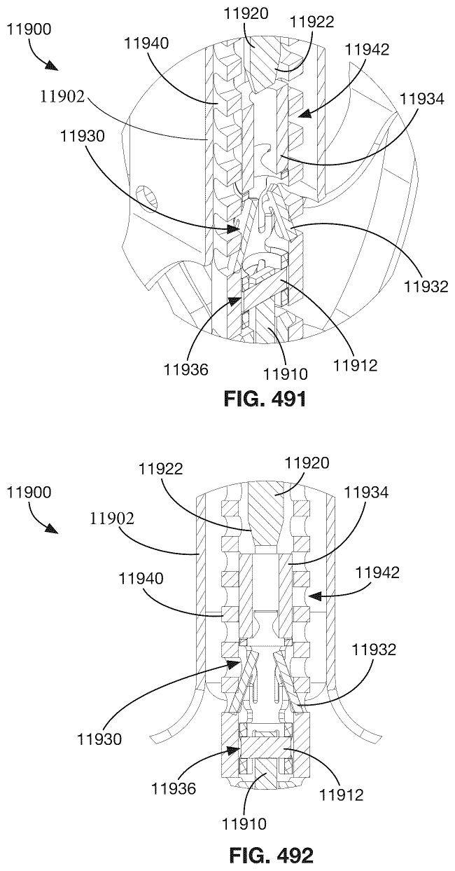

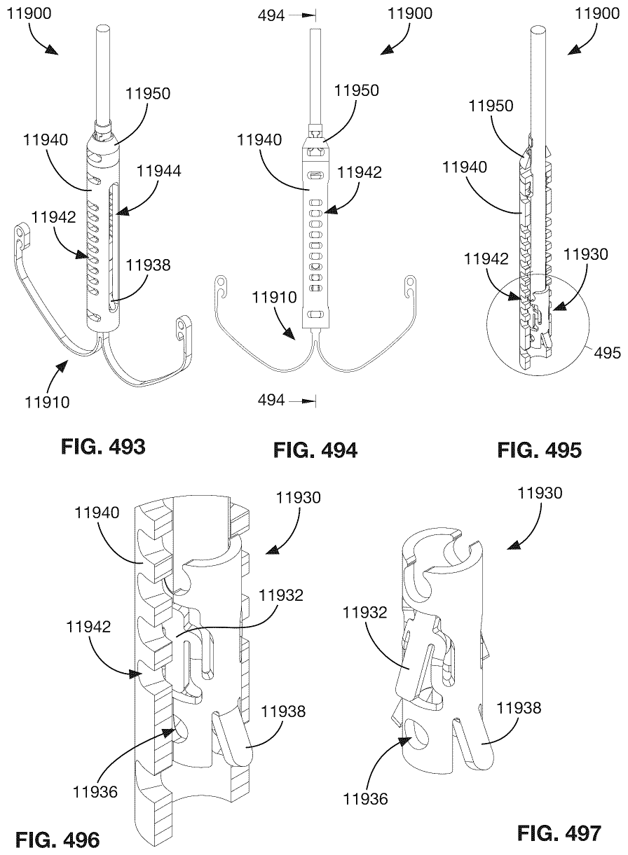

The actuation member 11920 (e.g., wire, rod, tube, shaft, conduit, etc.) has a tapered end 11922 that can be inserted between the inwardly biased ends of the latch members 11932 to cause the latch members 11932 to pivot from the latched condition (FIGS. 491-492) to the unlatched condition (FIGS. 487-488). The actuation member 11920 is inserted into the latch tube 11930 such that a threaded portion 11924 of the actuation member 11920 engages corresponding threads 11934 of the latch tube 11930 to connect the actuation member 11920 to the latch tube 11930. As can be seen in FIG. 493, orientation tabs 11938 of the latch tube 11930 extend through orientation slots 11944 of the receiver 11940 to limit rotation of the latch tube 11930 during threading and unthreading of the actuation member 11920 and the latch tube 11930.

During implantation of the device 11901 the position of the connector 11910 is adjusted by extending and retracting the actuation member 11920. Once the desired position of the connector 11910 has been reached, the actuation member 11920 is unscrewed from the latch tube 11930 and retracted to disengage the actuation member 11920 from the latch tube 11930, thereby allowing the latch members 11932 to pivot into the latched condition to engage the openings 11942 of the receiver 11940. The actuation member 11920 can then be withdrawn from the actuation mechanism 11900.

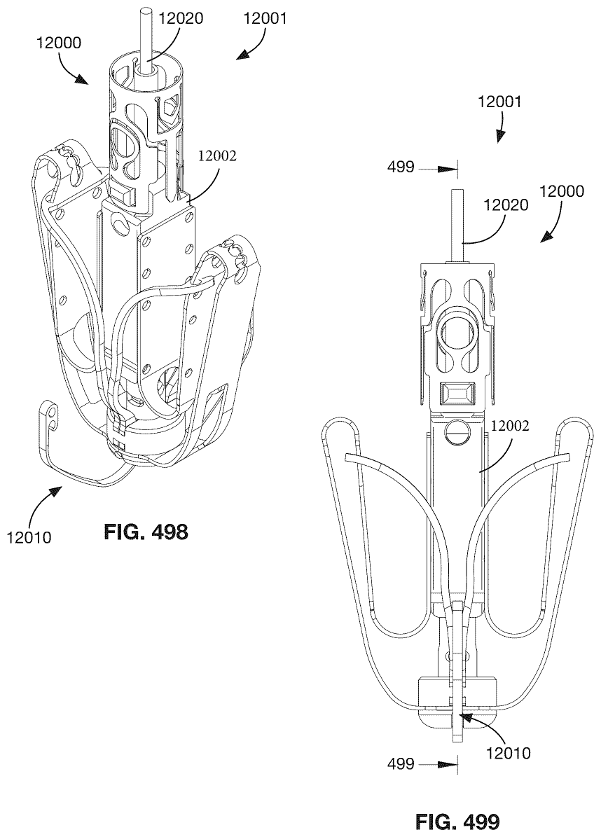

Referring now to FIGS. 498-516, an actuation mechanism 12000 for a device 12001 is shown. The actuation mechanism 12000 can be used with any of the devices described herein. The device 12001 can be opened and closed in the same manner as any of the implementations disclosed herein. In some implementations, an actuation element (e.g., any of the actuation elements disclosed herein) is coupled to a head 12050 that is threadably or otherwise attached to a receiver 12040 (e.g., an internally threaded element, a notched receiving portion, a column, a lumen, a tube, a shaft, a sleeve, a post, a housing, tracks, a cylinder, etc.). The head 12050 and attached receiver 12040 are advanced and retracted relative to a body or coaptation element 12002 (e.g., spacer, coaption element, gap filler, etc.) of the device to open and close the paddles of the device.

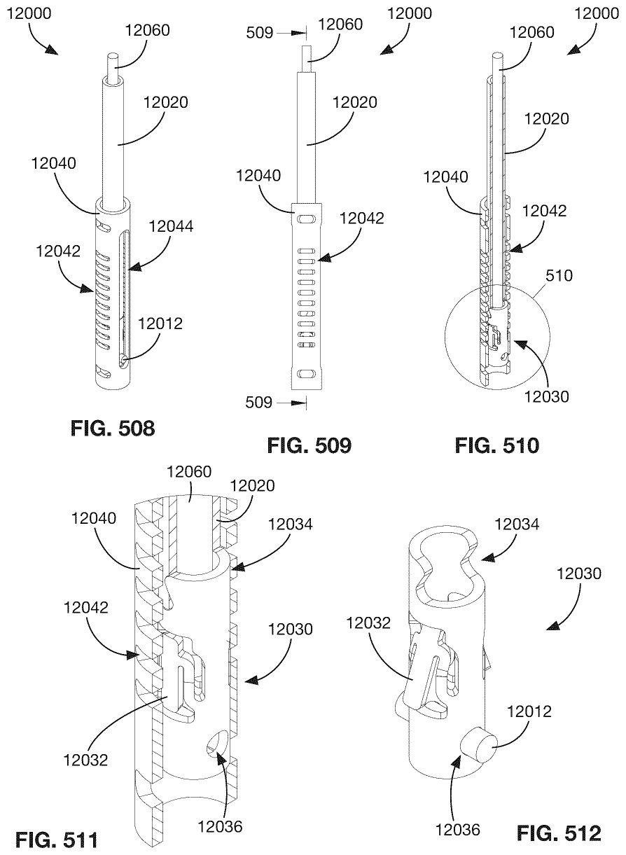

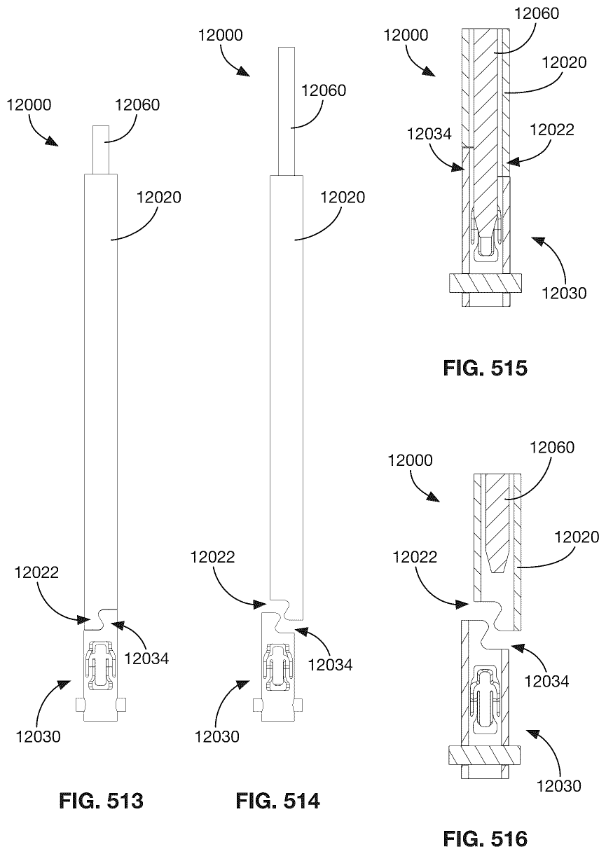

The actuation mechanism 12000 includes a paddle width adjustment connector 12010 (e.g., shaped metal component, shaped plastic component, tether, wire, strut, line, cord, suture, etc.) that is adjusted by a width adjustment element 12020 (e.g., width adjustment wire, width adjustment shaft, width adjustment tube, width adjustment line, width adjustment cord, width adjustment suture, etc.). The connector is attached to outer paddle frame portions (not shown in FIGS. 498-501) as described above. The width adjustment element 12020 extends to a distal end that is removably attached to a coupler, such as the illustrated latch tube 12030. A retention or securing member 12060 (e.g., wire, rod, tube, shaft, conduit, etc.) secures the connection between the width adjustment element 12020 and latch tube 12030; the relationship between the width adjustment element 12020, the latch tube 12030, and the securing member 12060 is shown in FIGS. 513-516 and described below. The latch tube 12030 is attached to the connector 12010 so that position of the connector 12010 can be adjusted by the width adjustment element 12020 via the latch tube 12030. The connector 12010 is attached to the latch tube 12030 via a pin 12012 that is received within openings 12036 near the distal end of the latch tube 12030. As can be seen in FIG. 508, the pin 12012 extends through the openings 12036 of the latch tube 12030 and through orientation slots 12044 of the outer tube or receiver 12040 to limit rotation of the latch tube 12030 during operation of the actuation mechanism 12000. The connector 12010 can be attached to the latch tube 12030 in any suitable way, such as, for example, via a weld connection, an adhesive, a threaded connection, or the like.

The width adjustment element 12020 and latch tube 12030 extend coaxially within an outer tube or receiver 12040 that is threadably or otherwise attached to a proximal head 12050. The outer tube or receiver 12040 can be attached to other components or mechanisms of the device 12001. For example, the outer tube or receiver 12040 can be advanced and retracted to open and close the paddles of the device. The proximal head 12050 can be engaged and actuated in a wide variety of ways described herein (such as, for example, via the mechanisms shown in FIGS. 417-482) to facilitate the implantation of the devices described herein, such as, for example, by opening and closing paddles of the device 12001.

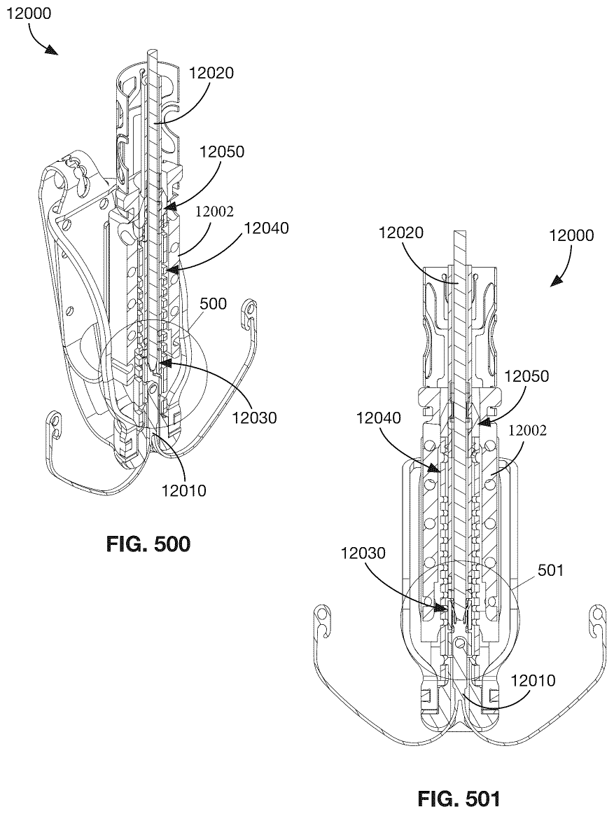

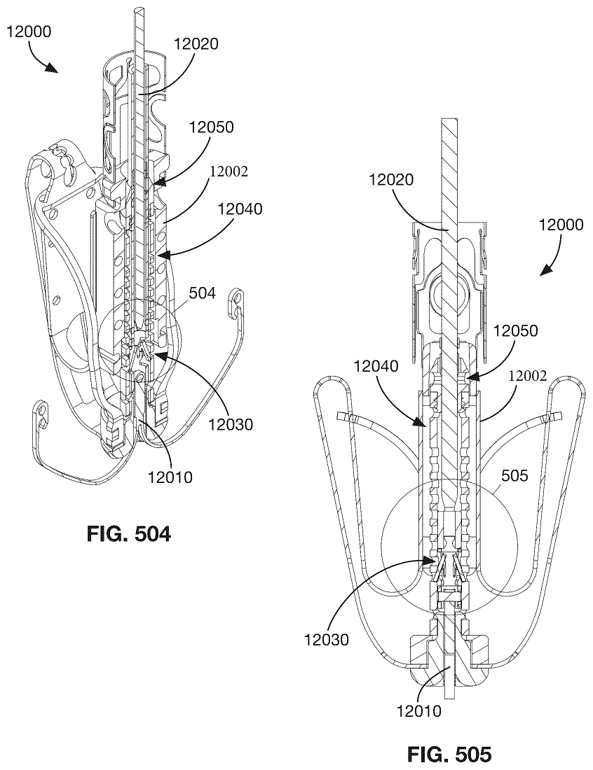

A coupler, such as the illustrated latch tube 12030 includes two latch members 12032 (e.g., arms, prongs, etc.) that can be moved between a latched and an unlatched condition. Referring now to FIGS. 500-503, the latch members 12032 of the latch tube 12030 are shown in an unlatched condition. When the latch members 12032 are in the unlatched condition, the latch tube 12030 can be moved freely along the length of the outer tube or receiver 12040 to extend and retract the connector 12010. In the latched condition, shown in FIGS. 504-507, the latch members 12032 in the latched condition engage openings 12042 of the outer tube or receiver 12040 to secure the position of the latch tube 12030 relative to the outer tube or receiver 12040. Consequently, the position of the connector 12010 relative to the paddles of the device 12001 is not change by actuation of the outer tube or receiver 12040 and proximal head 12050. That is, the width of the paddles frames is maintained as the paddles are opened and closed.

The latch tube 12030 can include any number of latch members 12032 suitable for engaging corresponding openings 12042 in the outer tube or receiver 12040. The latch members 12032 are integrally formed in the sides of the latch tube 12030 by laser-cutting portions of the latch tube 12030. The latch members 12032 are bent and shape-set in the latched condition so that in a free state the latch members 12032 are angled relative to a central axis of the latch tube 12030. For example, as is shown in FIG. 512, the proximal or upper ends of the latch members 12032 are biased radially inward and the opposite lower or distal ends of the latch members 12032 are biased radially outward. Alternatively, the proximal ends of the latch members 12032 can be biased radially outward and the opposite distal ends of the latch members 12032 can be biased radially inward. In some implementations, the connector 12010 is biased to extend or retract and the latch members 12032 are angled outward to oppose the bias of the connector 12010. For example, in an example where the connector 12010 is biased to move in the proximal direction, the proximal ends of the latch members 12032 can be biased radially outward so that the latch members 12032 engage the openings 12042 of the outer tube or receiver 12040 such that the proximal movement of the connector 12010 is resisted by the latch members 12032.

The width adjustment element 12020 extends to a distal end 12022 that is configured to form one half of a handshake-like coupling connection for engaging a proximal end 12034 of the latch tube 12030 that forms the other half of the coupling connection. The ends of the width adjustment element 12020 and the latch tube 12030 include angled surfaces that engage each other when the width adjustment element 12020 and latch tube 12030 are coupled such that moving the width adjustment element 12020 in the proximal direction to decouple the distal end 12022 from the proximal end 12034 causes the distal end 12022 of the width adjustment element 12020 to move laterally relative to the latch tube 12030. Thus, restricting relative lateral movement of the width adjustment element 12020 and latch tube 12030 prohibits the distal end 12022 of the width adjustment element 12020 from decoupling from the proximal end 12034 of the latch tube 12030.

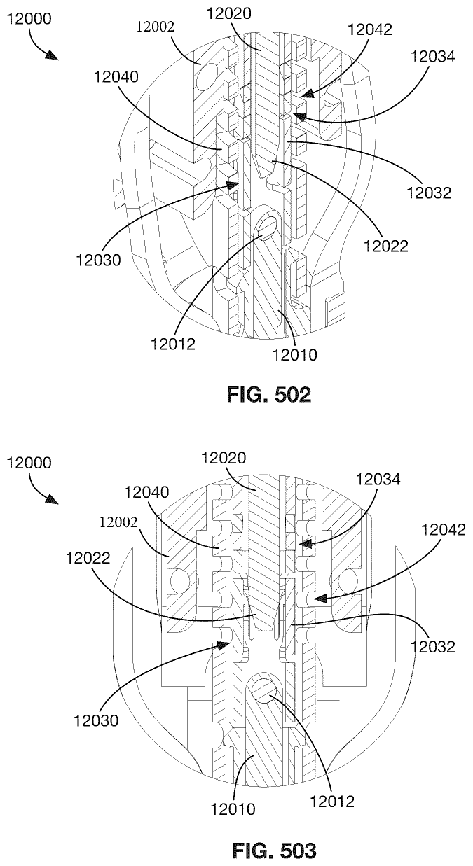

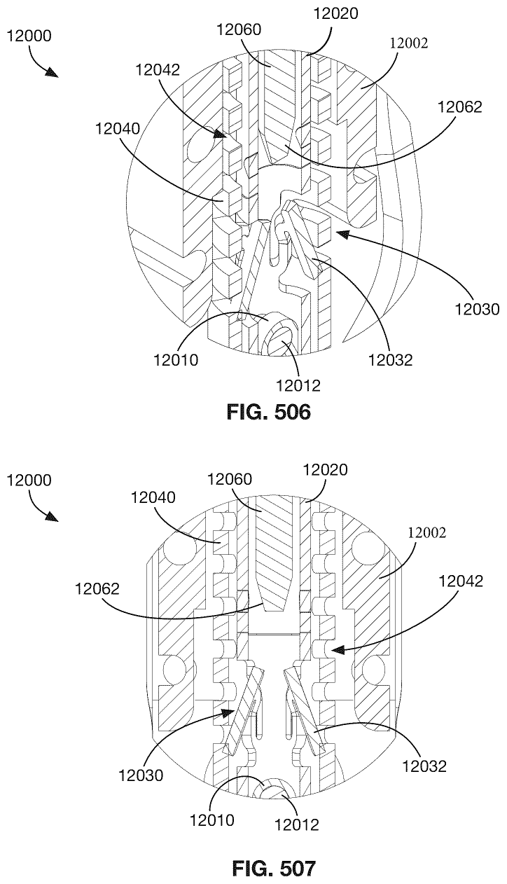

Such relative lateral movement can be prohibited by inserting the securing member 12060 through the width adjustment element 12020 and into the latch tube 12030. Retracting the securing member 12060 from the width adjustment element 12020 and the latch tube 12030 allows the distal end 12022 of the width adjustment element 12020 to move laterally away from the proximal end 12034 of the latch tube 12030 so that the width adjustment element 12020 can be decoupled from the latch tube 12030 via application of a proximal force. The securing member 12060 also has a tapered end 12062 that is inserted between the inwardly biased ends of the latch members 12032 to cause the latch members 12032 to pivot from the latched (FIGS. 506-507) to the unlatched (FIGS. 502-503) condition. Thus, the securing member 12060 secures the connection between the width adjustment element 12020 and the latch tube 12030 and is also used to move the latch members 12032 between the unlatched and latched condition. The securing member 12060 can be any suitable material, such as, for example, a nitinol wire.

During implantation of the device 12001 the position of the connector 12010 is adjusted by extending and retracting the width adjustment element 12020. Once the desired position of the connector 12010 has been reached, the securing member 12060 is retracted from the latch tube 12030 to allow the latch members 12032 to pivot into the latched condition to engage the openings 12042 of the outer tube or receiver 12040. Further retraction of the securing member 12060 from the latch tube 12030 allows the distal end 12022 of the width adjustment element 12020 to move laterally away from the proximal end 12034 of the latch tube 12030 so that the width adjustment element 12020 disengages from the latch tube 12030. The width adjustment element 12020 and securing member 12060 can then be withdrawn from the actuation mechanism 12000.

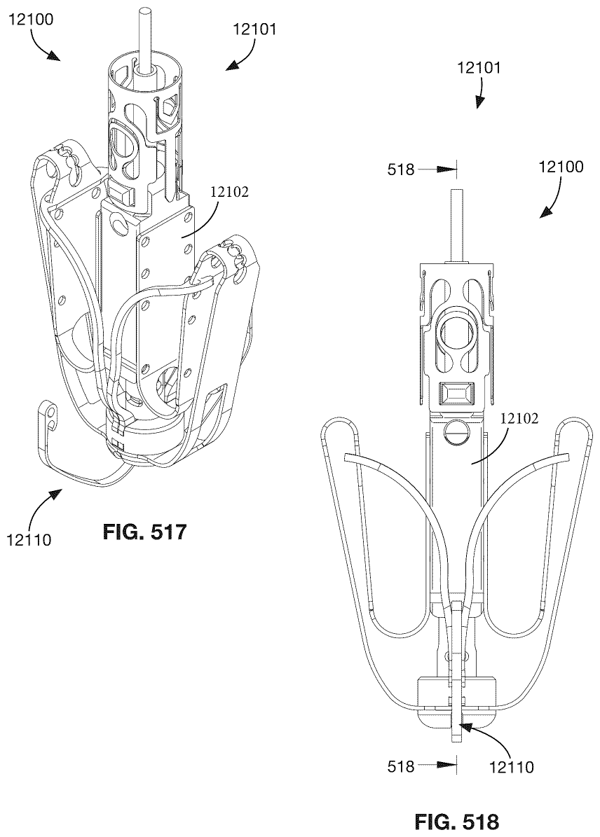

Referring now to FIGS. 517-534, an actuation mechanism 12100 for a device 12101 is shown. The actuation mechanism 12100 can be used with any of the devices described herein. The device 12101 can be opened and closed in the same manner as any of the implementations disclosed herein. In some implementations, an actuation element (e.g., any of the actuation elements disclosed herein) is coupled to a head 12150 that is threadably or otherwise attached to a receiver 12140 (e.g., an internally threaded element, a notched receiving portion, a column, a lumen, a tube, a shaft, a sleeve, a post, a housing, tracks, a cylinder, etc.). The head 12150 and attached receiver 12140 are advanced and retracted relative to a body or coaptation element 12102 (e.g., spacer, coaption element, gap filler, etc.) of the device to open and close the paddles of the device.

The actuation mechanism 12100 includes a paddle width adjustment connector 12110 (e.g., shaped metal component, shaped plastic component, tether, wire, strut, line, cord, suture, etc.) that is adjusted by a width adjustment element 12120 (e.g., width adjustment wire, width adjustment shaft, width adjustment tube, width adjustment line, width adjustment cord, width adjustment suture, etc.) that extends to a distal end and is removably attached to the connector 12110. The connector is attached to outer paddle frame portions (not shown in FIGS. 517-520) as described above. The position of the connector 12110 can be adjusted by extending and retracting the width adjustment element 12120. The width adjustment element 12120 can be a nitinol wire, a tube or other member that includes a handshake-like connection with the latch tube, or the like. In implementations where the width adjustment element 12120 is a suture or other flexible member, tension can be applied to the width adjustment element 12120 to retract the connector 12110 and tension can be released to allow the connector 12110 to extend by virtue of a biasing member (not shown) or an inherent bias in the connector 12110 established via, for example, shape setting.

The width adjustment element 12120 and proximal end of the connector 12110 extend coaxially within a receiver 12140 that, in turn, extend coaxially within the latch tube 12130 that is threadably or otherwise attached to a proximal head 12150. The latch tube 12130 can be attached to other components or mechanisms of the device 12101. The proximal head 12150 can be engaged and actuated in a wide variety of ways described herein (such as, for example, via the mechanisms shown in FIGS. 417-482) to facilitate the implantation of the devices described herein, such as, for example, by opening and closing paddles of the device 12101.

The latch tube 12130 includes a plurality of groups—for example, pairs—of latch members 12132 (e.g., arms, prongs, levers, etc.) that can be moved between a latched and an unlatched condition. The latch members 12132 are prohibited from moving from the unlatched to latched condition by a receiver 12140 that acts as a safety mechanism to prevent or inhibit the latch members 12132 from engaging corresponding grooves or notches 12112 in the connector 12110. The receiver 12140 extends within the latch tube 12130 between the connector 12110 and latch tube 12130 and includes an actuation portion 12142 that extends through the proximal head 12150 so that the receiver 12140 can be retracted in the proximal direction.

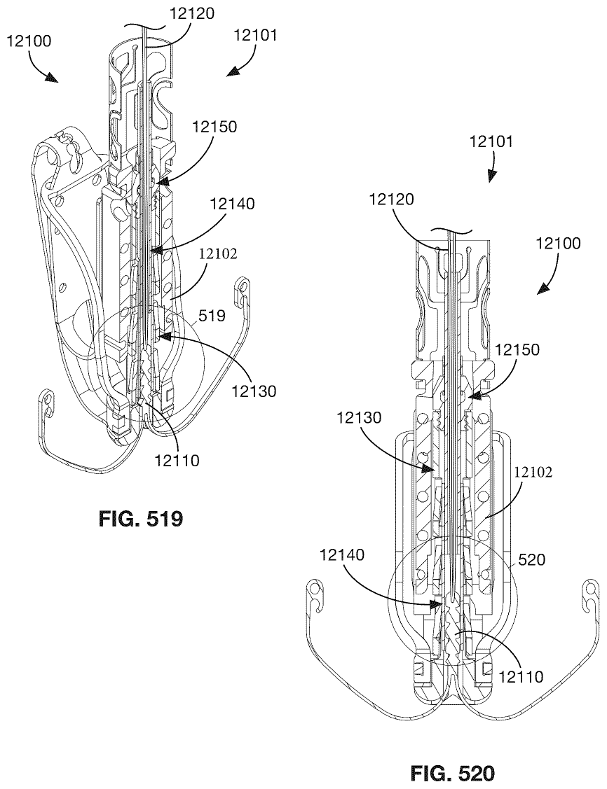

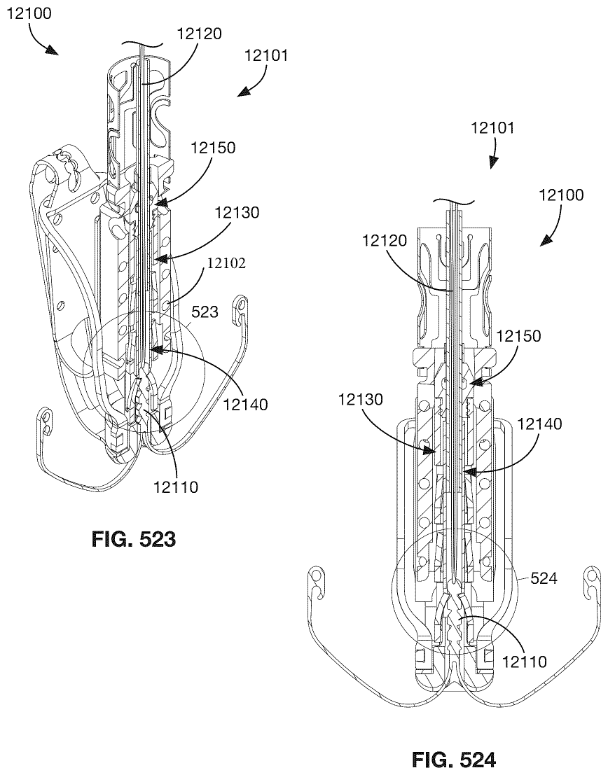

Referring now to FIGS. 519-522 and 527-530, the latch members 12132 of the latch tube 12130 are shown in an unlatched condition. When the latch members 12132 are in the unlatched condition, the proximal end of the connector 12110 can be moved freely along the length of the latch tube 12130 to extend and retract the connector 12110. In the latched condition, shown in FIGS. 523-526 and 531-534, the latch members 12032 in the latched condition engage the notches 12112 in the connector 12110 to secure the position of the connector 12110 relative to the latch tube 12130. Consequently, the position of the connector 12110 relative to the paddles of the device 12101 is not change by actuation of the latch tube 12130 and proximal head 12150.

The latch tube 12130 can include any number of latch members 12132 suitable for engaging the notches 12112 of the connector 12110. The latch members 12132 are integrally formed in the sides of the latch tube 12130 by laser-cutting portions of the latch tube 12130. The latch members 12132 are bent and shape-set in the latched condition so that in a free state the latch members 12132 are angled relative to a central axis of the latch tube 12130. For example, as is shown in FIG. 534, the free ends of the latch members 12132 are biased radially inward. The latch members 12132 are oriented to extend in a proximal or distal direction to oppose the movement of the connector 12110.

During implantation of the device 12101 the position of the connector 12110 is adjusted by extending and retracting the width adjustment element 12120. Once the desired position of the connector 12110 has been reached, the receiver 12140 is retracted from between the connector 12110 and the latch tube 12130 to allow the latch members 12132 to pivot into the latched condition to engage the notches 12112 of the connector 12110. Once side of the width adjustment element 12120 can be released so that further retraction of the width adjustment element 12120 detaches the actuation member from the connector 12110 so that the width adjustment element 12120 can be withdrawn from the actuation mechanism 12100.

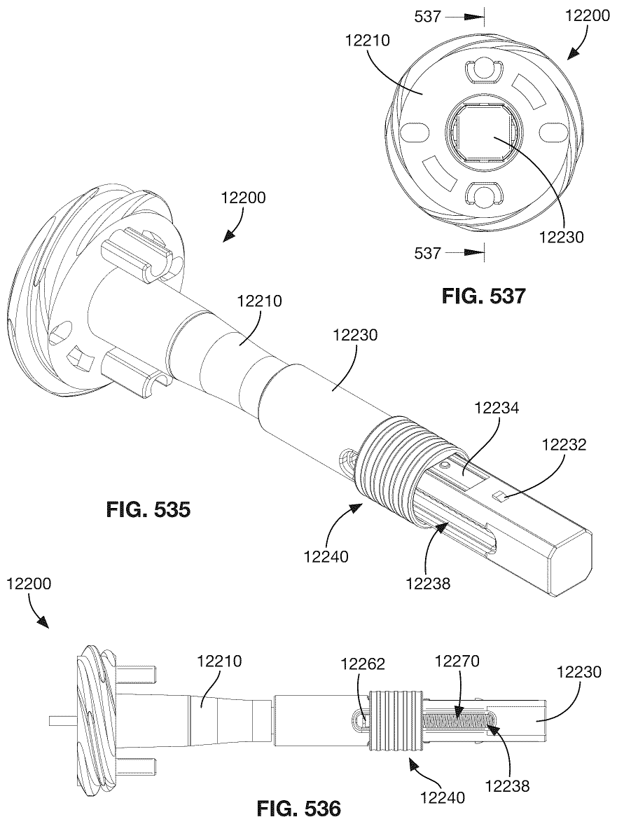

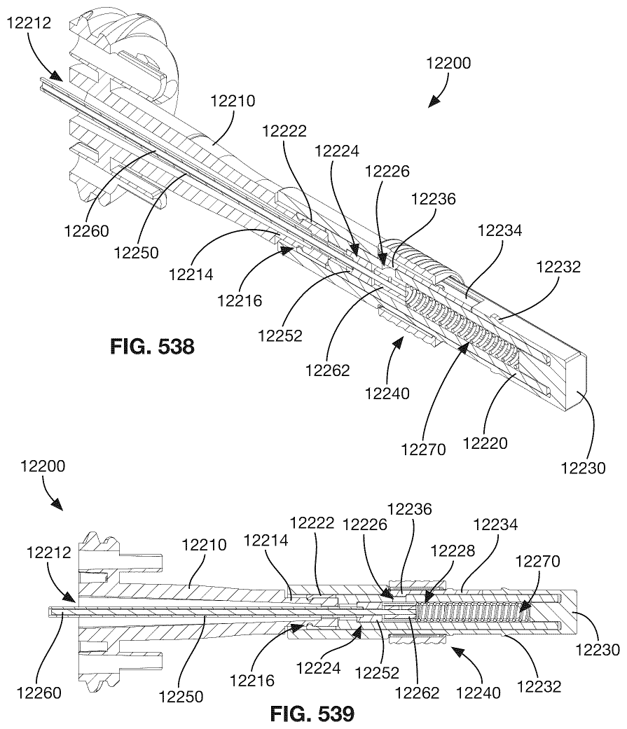

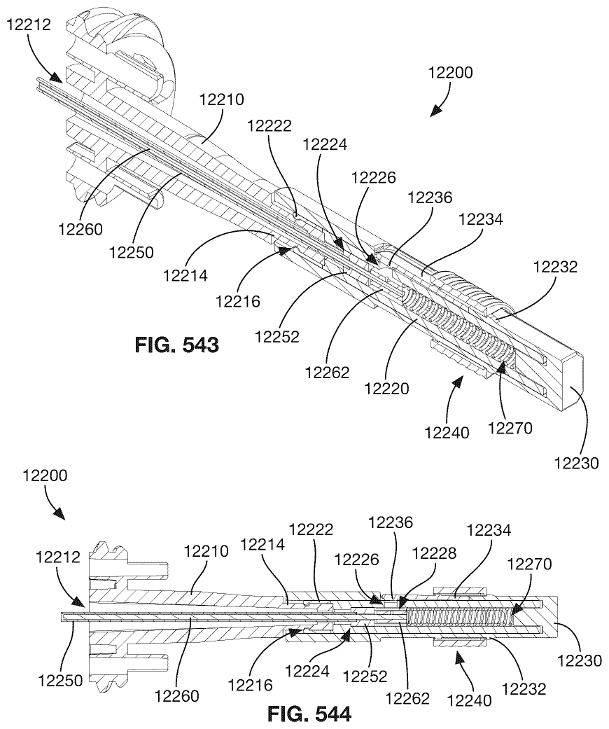

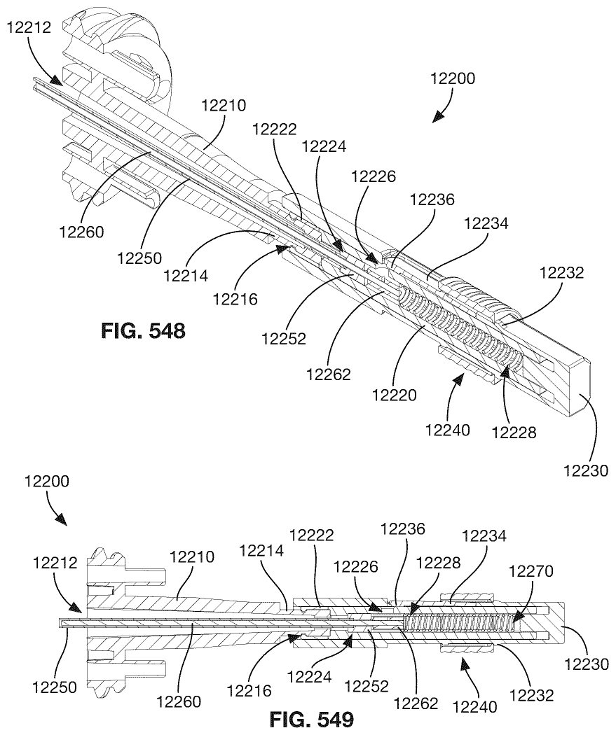

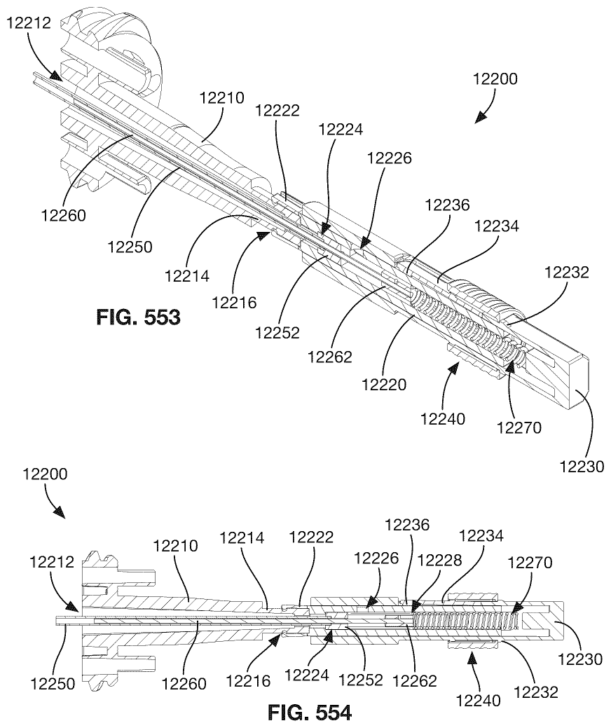

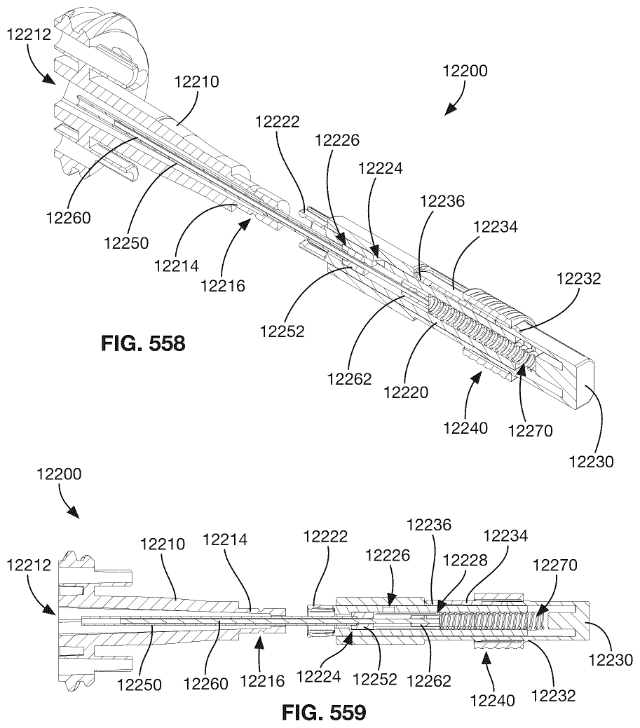

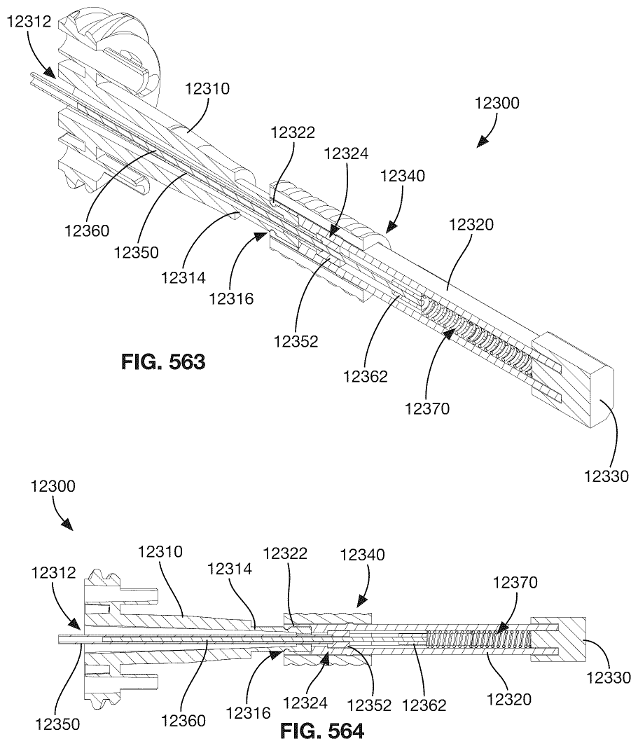

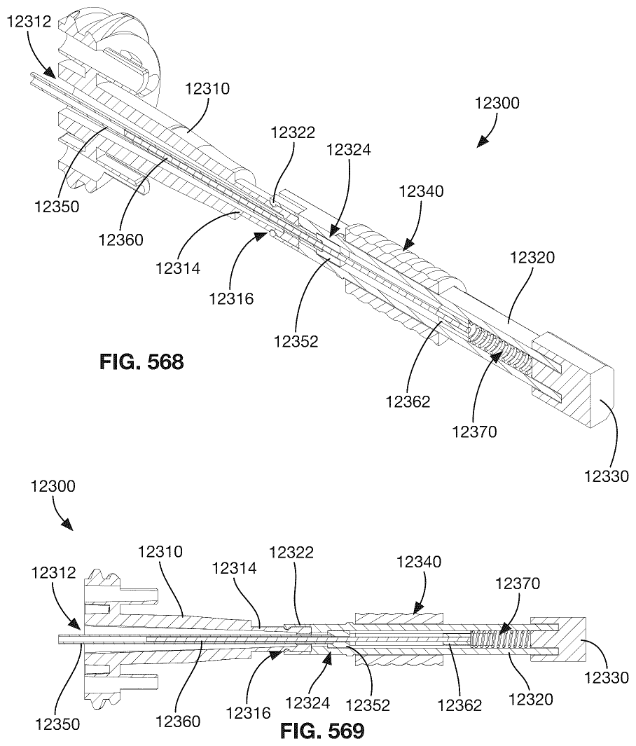

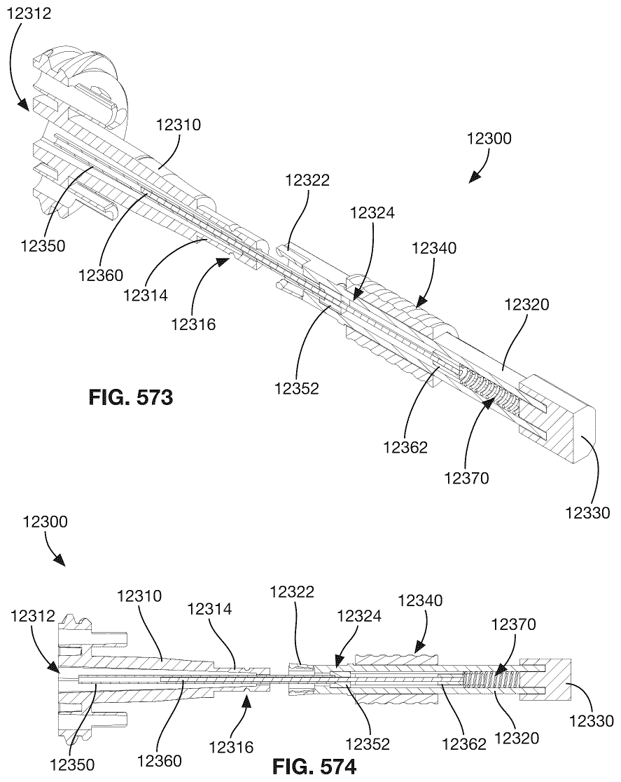

The devices disclosed herein can be deployed with a deployment system that includes a handle that allows the operator to actuate the various mechanisms of the device. Once the device has been delivered inside the patient, the operator can manipulate the handle to disengage the actuation element and/or the width adjustment element of the deployment system from the device and to withdraw the actuation element and/or the width adjustment element from the deployment system and ultimately, from the patient. The handle of the deployment system can include mechanisms for facilitating the disengagement and removal of the actuation element and/or the width adjustment element from the device and deployment system. Some implementations of handles for deployment systems are shown in FIGS. 535-574.

Referring now to FIGS. 535-559, a handle release assembly 12200 for a deployment system for deploying a device is shown. The handle release assembly 12200 can be used with any of the devices and deployment systems described herein. The handle release assembly can be used to decouple an actuation element 12250 (e.g., actuation shaft, actuation rod, actuation tube, actuation wire, etc.) and/or a width adjustment element 12260 (e.g., width adjustment wire, width adjustment shaft, width adjustment tube, width adjustment line, width adjustment cord, width adjustment suture, width adjustment screw or bolt etc.) from a device.

The handle release assembly 12200 includes a connector body 12210, an inner body 12220, and an outer body 12230. The connector body 12210 provides an interface between the release mechanism of the handle release assembly 12200 and a deployment system for deploying a device. The connector body 12210 is releasably connected to the inner body 12220. The outer body 12230 is moveably connected to the inner body 12220 and engages the interface between the connector body 12210 and the inner body 12220 so that the connection between the connector body 12210 and the inner body 12220 cannot decouple without the outer body 12230 being engaged first. A sliding gripping portion 12240 is provided on the outer body 12230 to allow the operator to manipulate the handle release assembly 12200 to withdraw an actuation element 12250 and a width adjustment element 12260 extending coaxially within the actuation element 12250. Actuation of the gripping portion 12240 also facilitates the release of the inner and outer bodies 12220, 12230 from the connector body 12210 so that the actuation element 12250 and width adjustment element 12260 can be withdrawn from the delivery system.

The connector body 12210 includes a central lumen 12212 through which the actuation element 12250 and width adjustment element 12260 extend. The connector body 12210 extends from a distal end that engages the deployment system to a proximal end that includes a coupling portion 12214. The coupling portion 12214 includes a retention groove 12216 that receives attachment members 12222 (e.g., prongs, arms, collet, ferrule, etc.) at a distal end of the inner body 12220. The coupling portion 12214 has a cylindrical shape and the attachment members 12222 slidably engage the annular retention groove 12216 to enable rotation of the inner body 12220 relative to the connector body 12210 while the inner body 12220 and connector body 12210 are connected.

The outer body 12230 receives the inner body 12220 in a central bore and the gripping portion 12240 surrounds a portion of the outer body 12230. The inner body 12220 includes a recess or catch 12226 for receiving a corresponding protrusion 12236 of a flexible latch arm 12234 of the outer body 12230. The engagement of the protrusion 12236 in the catch 12226 prohibits the outer body 12230 from moving relative to the inner body 12220 during normal use, as can be seen in FIGS. 535-539. The gripping portion 12240 covers the flexible latch arm 12234 to hold the flexible latch arm 12234 against the inner body 12220, thereby maintaining the engagement of the protrusion 12236 with the catch 12226. The operator can grip the gripping portion 12240 and outer body 12230 to rotate the inner body 12220, the outer body 12230, and the gripping portion 12240 relative to the connector body 12210; this rotation also causes the actuation element 12250 and width adjustment element 12260 to rotate relative to the connector body 12210 and the deployment system. A spring 12270 is provided inside the inner body 12220 and extends between the proximal end of the outer body 12230 and the width adjustment element adaptor 12262. The spring 12270 is biased in the expansion direction to prohibit unintended proximal movement of the width adjustment element 12260 and the actuation element 12250, thereby retaining the components of the handle release assembly 12200 in the unreleased condition (FIGS. 535-539) until acted upon by the operator.

The actuation element 12250 extends from a distal end that engages a proximal head of a device proximally to a proximal end that is secured to an actuation element adaptor 12252. The actuation element adaptor 12252 fits within a recess 12224 of the inner body 12220. The recess 12224 is shaped to receive and prohibit the rotation and distal movement of the actuation element adaptor 12252. Such as, for example, a square shape or a round shape with corresponding flat surfaces. Consequently, the actuation element 12250 can be rotated by rotating the inner body 12220 and the actuation element 12250 can be retracted in a proximal direction when the inner body 12220 is moved in a proximal direction, thereby applying tension to the actuation element 12250. The actuation element 12250 can be rotated in this way to actuate a rotatably actuated mechanism of the device and can be retracted after the device has been deployed.

The width adjustment element 12260 extends from a distal end that engages the device proximally to a proximal end that is secured to a width adjustment element adaptor 12262. The width adjustment element adaptor 12262 extends laterally through side slots 12228 of the inner body 12220 and through corresponding side slots 12238 of the outer body 12230. The width adjustment element adaptor 12262 protrudes from the side slots 12238 of the outer body 12230 so that the width adjustment element adapter 12262 can be engaged by movement of the side slots 12238 in the proximal direction. The width adjustment element adaptor 12262 is also prohibited from rotating by the sides of the side slots 12228, 12238 and can, consequently, be rotated by rotating the inner and outer bodies 12220, 12230, thereby causing the width adjustment element 12260 to rotate. In this way, the width adjustment element 12260 can be rotated, for example, to disengage the distal end of the width adjustment element 12260 from a threaded connection with the device. The width adjustment element adaptor 12262 can also be moved proximally to retract the width adjustment element 12260 after the device has been deployed.

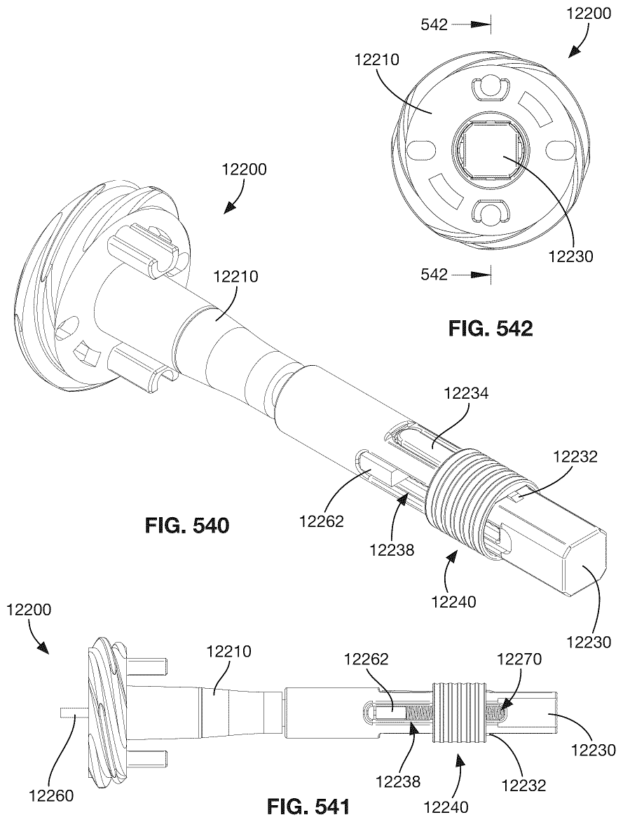

The handle release assembly 12200 can be rotated as described above to rotate the actuation element 12250 and the width adjustment element 12260 to actuate various mechanisms of the device. To complete the implantation procedure, the actuation element 12250 and the width adjustment element 12260 are decoupled from the implanted device and retracted from the delivery system. To accomplish the retraction of the actuation element 12250 and the width adjustment element, the inner body 12220 is decoupled from the connector body 12210 so that the actuation element 12250 and the width adjustment element 12260 can be fully retracted from the delivery system. The position of the various components during actuation of the mechanisms that release the inner body 12220 from the connector body 12210 of the handle release assembly 12200 are shown in FIGS. 540-559.

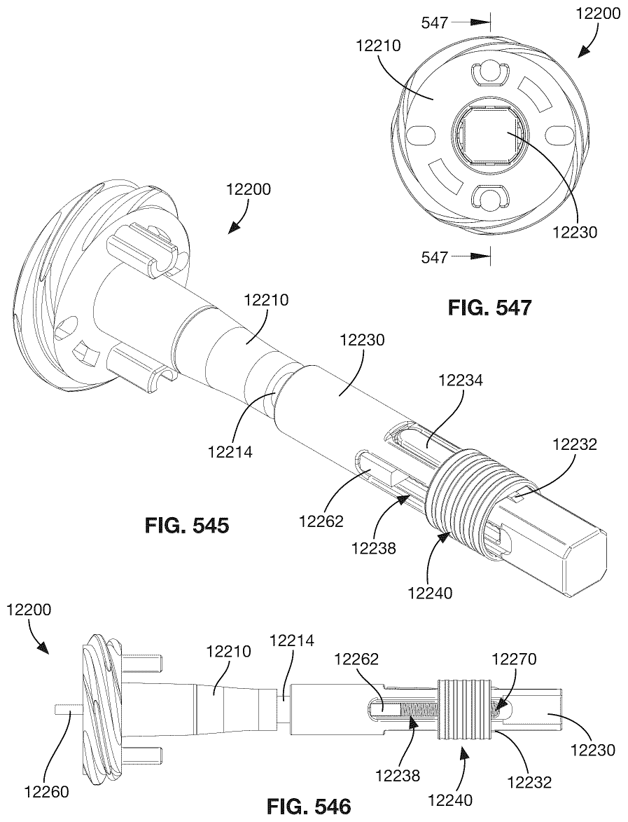

Referring now to FIGS. 540-544, the release action of the handle release assembly 12200 is initiated by the operator sliding the gripping portion 12240 in the proximal direction along the outer body 12230 until the gripping portion 12240 engages a pair of proximal stops 12232. Moving the gripping portion 12240 in this way exposes the latch arm 12234 of the outer body 12230 and allows the latch arm 12234 to bend outwards so that the protrusion 12236 can disengage from the catch 12226. The protrusion 12236 can include rounded features that facilitate disengagement from the catch 12226 as a result of proximal movement of the outer body 12230 relative to the inner body 12220. The latch arm 12234 can also be biased in an outward direction so that the latch arm 12234 flexes outward as the gripping portion 12240 is moved. Pulling the gripping portion 12240 further in the proximal direction applies a proximal force to the stops 12232 of the outer body 12230 to move the now-released outer body 12230 in the proximal direction relative to the inner body 12220 and the connector body 12210, as can be seen in FIGS. 545-549 to disengage the protrusion 12236 from the catch 12226.

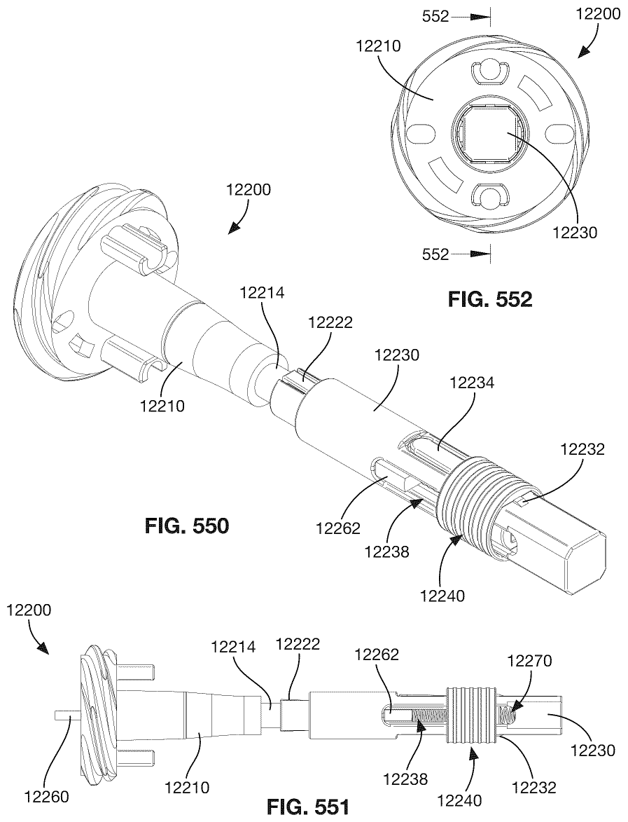

Referring now to FIGS. 550-554, further proximal movement of the gripping portion 12240 engages the stops 12232 of the outer body 12230 and pushes the outer body 12230 in the proximal direction. During the proximal movement of the outer body 12230 the side slots 12238 of the outer body 12230 engage the protruding ends of the width adjustment element adaptor 12262 to pull the width adjustment element adaptor 12262 and the width adjustment element 12260 in the proximal direction. Proximal movement of the outer body 12230 also exposes the attachment members 12222 of the inner body 12220. Once exposed, the attachment members 12222 can flex outward so that further proximal movement of the inner body 12220 causes the attachment members 12222 to disengage from the retention groove 12216 of the coupling portion 12214 of the connector body 12210. In some implementations, the attachment members 12222 can be biased in the outward direction so that attachment members 12222 flex outwards immediately upon being uncovered by proximal movement of the outer body 12230.

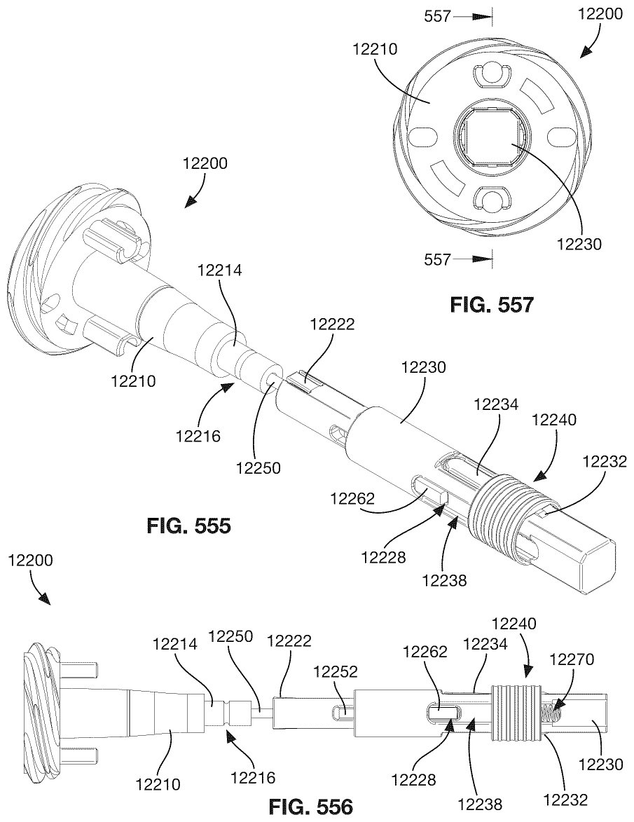

Referring now to FIGS. 555-559, the gripping portion 12240 and the outer body 12230 are moved in the proximal direction so that the width adjustment element adaptor 12262 engages the proximal end of the side slots 12228 in the inner body 12220 and the proximally directed pulling force is transferred to the inner body 12220. Applying a proximal force to the inner body 12220 causes the attachment members 12222 to spread apart and disengage from the retention groove 12216 of the coupling portion 12214 to decouple the inner body 12220 from the connector body 12210. Decoupling the inner body 12220 from the connector body 12210 facilitates proximal movement of the inner body 12220 away from the connector body 12210 so that the actuation element adaptor 12252 that is retained in the recess of the inner body 12220 can be moved proximally to pull the actuation element 12250 proximally. In this way, the actuation element 12250 and the width adjustment element 12260 can be disengaged and withdrawn from the device and deployment system after delivery into the patient.

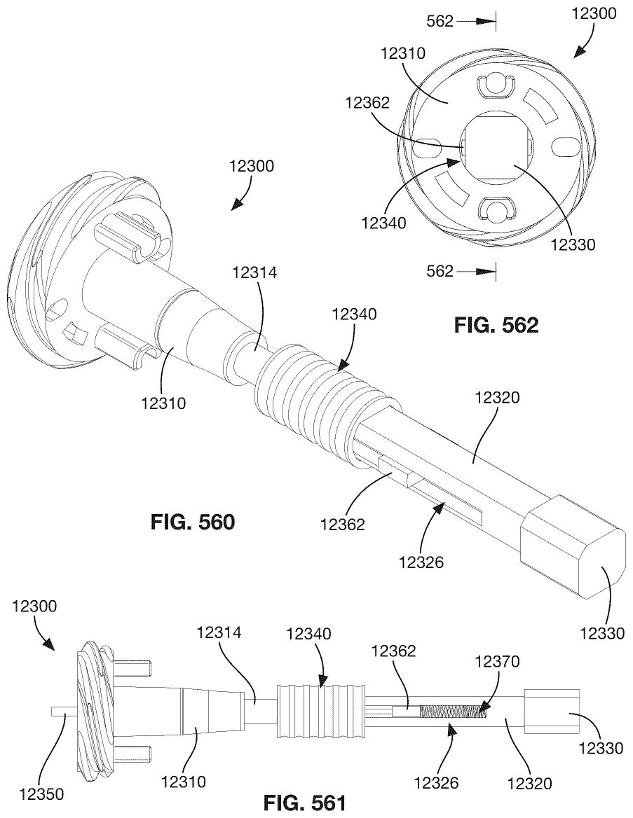

Referring now to FIGS. 560-574, a handle release assembly 12300 for a deployment system for deploying a device is shown. The handle release assembly 12300 can be used with any of the devices and deployment systems described herein. The handle release assembly can be used to decouple an actuation element 12350 (e.g., actuation shaft, actuation rod, actuation tube, actuation wire, etc.) and/or a width adjustment element 12360 (e.g., width adjustment wire, width adjustment shaft, width adjustment tube, width adjustment line, width adjustment cord, width adjustment suture, width adjustment screw or bolt etc.) from a device.

The handle release assembly 12300 includes a connector body 12310 and a moveable body 12320. The connector body 12310 provides an interface between the release mechanism of the handle release assembly 12300 and a deployment system for deploying a device. The connector body 12310 is releasably connected to the moveable body 12320. A sliding gripping portion 12340 is provided on the moveable body 12320 to allow the operator to manipulate the handle release assembly 12300 to withdraw an actuation element 12350 and a width adjustment element 12360 (e.g., a nitinol wire) extending coaxially within the actuation element 12350. Actuation of the gripping portion 12340 also facilitates the release of the moveable body 12320 from the connector body 12310 so that the actuation element 12350 and width adjustment element 12360 can be withdrawn from the delivery system.

The connector body 12310 includes a central lumen 12312 through which the actuation element 12350 and width adjustment element 12360 extend. The connector body 12310 extends from a distal end that engages the deployment system to a proximal end that includes a coupling portion 12314. The coupling portion 12314 includes a retention groove 12316 that receives attachment members 12322 (e.g., prongs, arms, collet, ferrule, etc.) at a distal end of the moveable body 12320. The coupling portion 12314 has a cylindrical shape and the attachment members 12322 slidably engage the annular retention groove 12316 to enable rotation of the moveable body 12320 relative to the connector body 12310 while the moveable body 12320 and connector body 12310 are connected. The operator can grip the gripping portion 12340 and moveable body 12320 to rotate the moveable body 12320 and the gripping portion 12340 relative to the connector body 12310; this rotation also causes the actuation element 12350 and width adjustment element 12360 to rotate relative to the connector body 12310 and the deployment system. A spring 12370 is provided inside the moveable body 12320 and extends between a proximal cap 12330 attached to the proximal end of the moveable body 12320 and the width adjustment element adaptor 12362. The spring 12370 is biased in the expansion direction to prohibit unintended proximal movement of the width adjustment element 12360 and the actuation element 12350, thereby retaining the components of the handle release assembly 12300 in the unreleased condition (FIGS. 560-564) until acted upon by the operator.

The actuation element 12350 extends from a distal end that engages a proximal head of a device proximally to a proximal end that is secured to an actuation element adaptor 12352. The actuation element adaptor 12352 fits within a recess 12324 of the inner body 12320. The recess 12324 is shaped to receive and prohibit the rotation and distal movement of the actuation element adaptor 12352. Such as, for example, a square shape or a round shape with corresponding flat surfaces. Consequently, the actuation element 12350 can be rotated by rotating the moveable body 12320 and the actuation element 12350 can be retracted in a proximal direction when the moveable body 12320 is moved in a proximal direction, thereby applying tension to the actuation element 12350. The actuation element 12350 can be rotated in this way to actuate a rotatably actuated mechanism of the device and can be retracted after the device has been deployed.

The width adjustment element 12360 extends from a distal end that engages the device proximally to a proximal end that is secured to a width adjustment element adaptor 12362. The width adjustment element adaptor 12362 extends laterally through side slots 12326 of the moveable body 12320. The width adjustment element adaptor 12362 protrudes from the side slots 12326 of the moveable body 12320 so that the width adjustment element adapter 12362 can be engaged by movement of the gripping portion 12340. The width adjustment element adaptor 12362 is also prohibited from rotating by the sides of the side slots 12326 and can, consequently, be rotated by rotating the moveable body 12320, thereby causing the width adjustment element 12360 to rotate. In this way, the width adjustment element 12360 can be rotated, for example, to disengage the distal end of the width adjustment element 12360 from a threaded connection with the device. The width adjustment element adaptor 12362 can also be moved proximally to retract the width adjustment element 12360 after the device has been deployed.

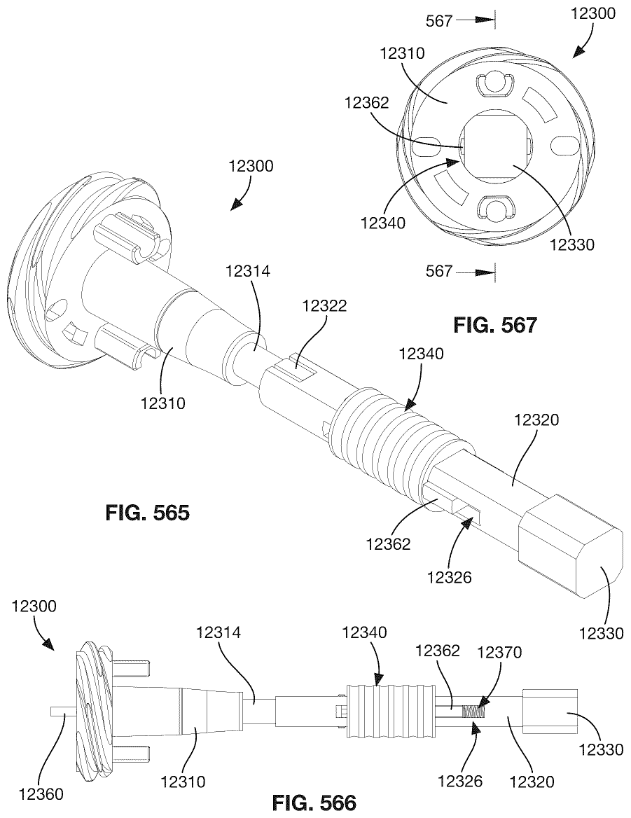

The handle release assembly 12300 can be rotated as described above to rotate the actuation element 12350 and the width adjustment element 12360 to actuate various mechanisms of the device. To complete the implantation procedure, the actuation element 12350 and the width adjustment element 12360 are decoupled from the implanted device and retracted from the delivery system. To accomplish the retraction of the actuation element 12350 and the width adjustment element, the inner body 12320 is decoupled from the connector body 12310 so that the actuation element 12350 and the width adjustment element 12360 can be fully retracted from the delivery system. The position of the various components during actuation of the mechanisms that release the moveable body 12320 from the connector body 12310 of the handle release assembly 12300 are shown in FIGS. 565-574.

Referring now to FIGS. 565-569, the release action of the handle release assembly 12300 is initiated by the operator sliding the gripping portion 12340 in the proximal direction along the moveable body 12320 until the gripping portion 12340 engages the ends of the width adjustment element adaptor 12362 that extend through the side slots 12326. Further movement of the gripping portion 12340 while engaged with the width adjustment element adaptor 12362 pulls the width adjustment element adaptor 12362 and the width adjustment element 12360 in the proximal direction. Moving the gripping portion 12340 in this way exposes the attachment members 12322 of the moveable body 12320. Once exposed, the attachment members 12322 can flex outward so that further proximal movement of the moveable body 12320 causes the attachment members 12322 to disengage from the retention groove 12316 of the coupling portion 12314 of the connector body 12310. In some implementations, the attachment members 12322 can be biased in the outward direction so that attachment members 12322 flex outwards immediately upon being uncovered by proximal movement of the gripping portion 12340.

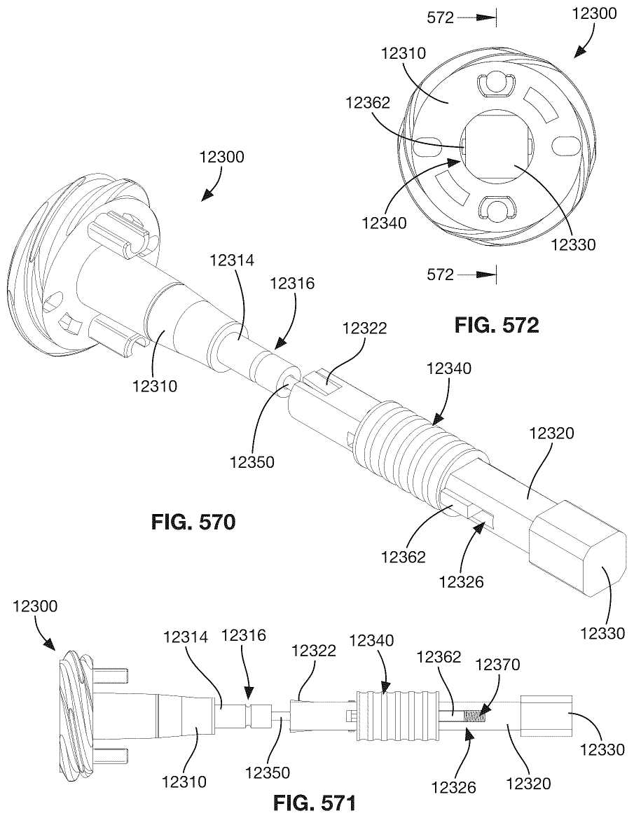

Referring now to FIGS. 570-574, the gripping portion 12340 and the moveable body 12320 are moved in the proximal direction until the width adjustment element adaptor 12362 pushes against the spring 12370 and/or engages the proximal end of the side slots 12326 of the moveable body 12320 so that the proximally directed pulling force is transferred to the moveable body 12320. Applying a proximal force to the moveable body 12320 causes the attachment members 12322 to spread apart and disengage from the retention groove 12316 of the coupling portion 12314 to decouple the inner body 12320 from the connector body 12310. Decoupling the moveable body 12320 from the connector body 12310 facilitates proximal movement of the moveable body 12320 away from the connector body 12310 so that the actuation element adaptor 12352 that is retained in the recess 12324 of the inner body 12320 can be moved proximally to pull the actuation element 12350 proximally. In this way, the actuation element 12350 and the width adjustment element 12360 can be disengaged and withdrawn from the device and deployment system after delivery into the patient.

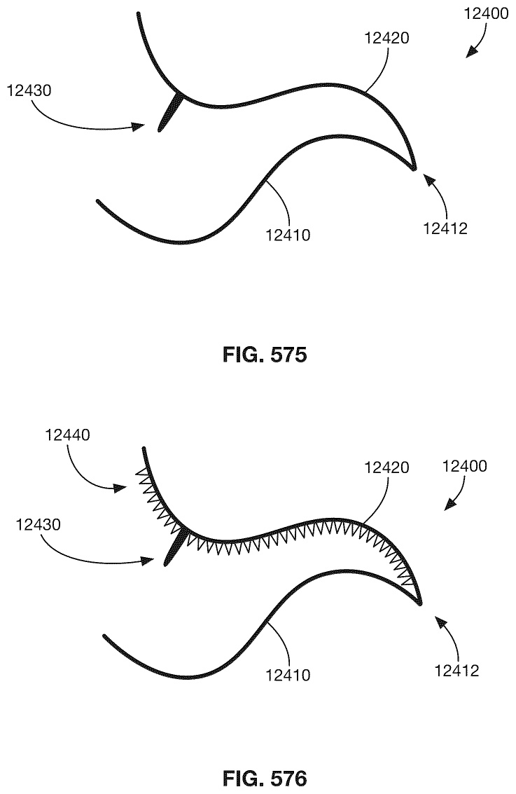

As has been described herein, gripping members or clasps can be used in a device to securely attach the device to the native tissue of the valve leaflets. (See, e.g., FIGS. 38-49 and the accompanying description above.) The arms of the clasps described above typically have a flat surface and can include a friction enhancing portion (e.g., barbs, protrusions, ridges, grooves, textured surfaces, adhesive, etc.) of the clasps 130 to grab onto the tissue of the valve leaflets. Some implementations of example clasps with arms having bent, curved, and/or textured surfaces are shown in FIGS. 575-582. The curving and texturing of the arms of the clasps increases the surface area of the clasp engaging the leaflet tissue, thereby increasing the surface area through which clasping forces are applied to the native tissue of the heart valve. Increasing the surface area of the clasps that contacts the native tissue improves the gripping strength or resistance to leaflet pull-out of the clasp. That is, the increased surface area spreads out the clasp gripping force across a greater amount of the leaflet tissue so that each piece of tissue experiences a lower gripping pressure even when the gripping force of the clasp is increased. Consequently, gripping forces can be increased overall until the gripping pressure in each portion of the clasp reaches the point of causing damage to the native tissue.

Referring now to FIGS. 575-576, an example clasp 12400 for a device is shown. The clasp 12400 includes a base or fixed arm 12410, a movable arm 12420, and a joint portion 12412. The fixed arm 12410 can be attached to an inner paddle of the device, with the joint portion 12412 disposed proximate the coaptation element. (See, e.g., FIGS. 26-27.) The joint portion 12412 can be spring-loaded so that the fixed and moveable arms 12410, 12420 are biased toward each other when the clasp 12400 is in a closed condition.

Like the various clasps described above, the fixed arm 12410 can be attached to the inner paddle of the device with any suitable means, such as screws or other fasteners, crimped sleeves, mechanical latches or snaps, welding, adhesive, or the like. The fixed arm 12410 remains substantially stationary relative to the inner paddle when the moveable arm 12420 is opened to open the clasp 12400 to capture a leaflet. The clasp 12400 can be opened by applying tension to actuation lines (e.g., the actuation lines 216 shown in FIGS. 43-48) attached to the moveable arms 12420, thereby causing the moveable arms 12420 to pivot or flex on the joint portions 12412.

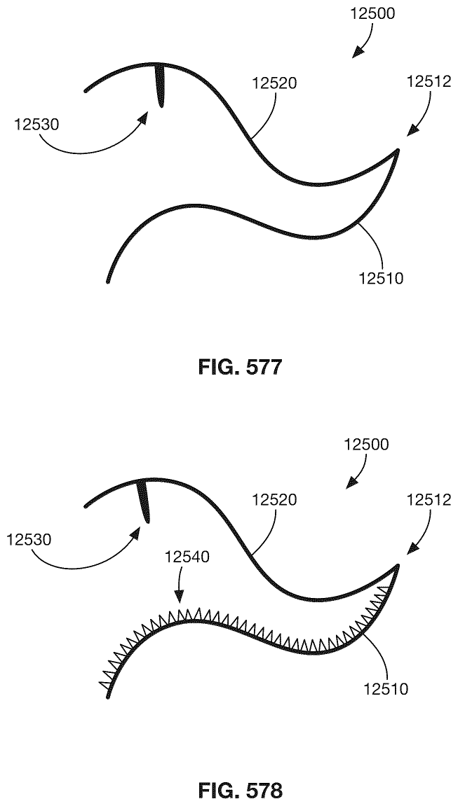

The clasp 12400 can include a variety of features that increase friction between the fixed and moveable arms 12410, 12420 and the tissue of the leaflet to better retain the leaflet within the clasp. For example, the fixed arm 12410 and the moveable arm 12420 can be formed to have a complementary curved or wavy shape, such as the sinusoidal pattern shown in FIGS. 577-578. The sinusoidal pattern of the fixed arm 12410 and moveable arm 12420 has a peak arranged near the joint portion 12412 and a trough arranged near the distal ends of the fixed arm 12410 and the moveable arm 12420. The curved shape of the fixed arm 12410 and the moveable arm 12420 provides an increased gripping surface between the joint portion 12412 and the distal ends of the fixed arm 12410 and the moveable arm 12420 than a straight configuration.

The clasp 12400 also includes a gripping protrusion 12430 that extends from the moveable arm 12420 and that provides a discrete increase in friction between the clasp 12400 and the leaflet at a predetermined location along the length of the fixed arm 12410 and the moveable arm 12420. The gripping protrusion 12430 is similar to the barbed portions described herein but can have a blunt or rounded end (as shown in FIG. 575) to reduce the potential that gripping the leaflet will cause tissue damage. The gripping protrusion 12430 can be arranged on the moveable arm 12420 as shown or can extend from the fixed arm 12410. Additional gripping protrusions can also be provided at other locations along the fixed arm 12410 and the moveable arm 12420.

A gripping surface 12440 can optionally be included on one or both of the fixed arm 12410 and the moveable arm 12420 as an optional third friction-increasing feature, as shown in FIG. 576. The gripping surface 12440 includes surface features that increase friction retaining force against the leaflet and can take on a wide variety of forms. For example, the gripping surface 12440 can be formed from a plurality of miniature teeth or serrations, such as those shown in FIG. 576. The miniature teeth or serrations can be angled toward an apex at which the clasp and inner paddle intersect to increase retention force against the leaflet. The gripping surface 12440 can also include—in addition to or instead of the teeth or serrations—a coating that increases the friction between the leaflet tissue and the fixed arm 12410 and the moveable arm 12420.

In some implementations of the clasp 12400 that include a plurality of miniature teeth or serrations, the surface features can be formed in a wide variety of ways. Such as, for example, a powder coating process in which a powder from which the surface features will be generated is sprayed on the metallic surfaces while applying high voltage to the surface, thereby creating an electric field in the direction the surface features—e.g., serrations—are facing. Alternatively, a procedure similar to laser printing can also be used to create friction-increasing surface features. In such an example process, a positively charged drum is passed over by a laser that reverses the charges in the areas the laser hits. A roller that includes the particles which will become the surface features is then passed over the drum so that the particles stick to the negatively charged drum. The coated drum can be then passed over the surfaces of the fixed arm 12410 and the moveable arm 12420 to form the friction-increasing surface features of the gripping surface 12440. Powder coating or laser printing processes can be used to provide a desired size and orientation of the surface retention features to increase engagement with the native tissue of the leaflet while also enabling disengagement from the leaflet tissue when desired. As has been described herein, the clasps 12400 can be covered by a cloth to reduce damage to the tissue that may result from direct contact with the metal and also to allow blood flow over the cells of the leaflet tissue through the gaps between the cloth fibers. Thus, the retention features of the gripping surface 12440 are preferably dimensioned to provide increased retention of the leaflet through the cloth covering. Alternatively, the retention features of the gripping surface 12440 can be formed over the cloth covering via the procedures—i.e., laser-printing based—described above.

The retention features of the gripping surface 12440 combined with the curved surfaces of the fixed and the moveable arms 12410, 12420 provide both micro-level and macro-level solutions for increasing the gripping force exerted on the leaflet by the clasp 12400. Because of the increased friction of these other features, the gripping protrusion 12430 can be smaller and/or have a less sharp tip than comparable barbed portions of other clasps so that the gripping protrusion 12430 is less likely to penetrate the native tissue.

Referring now to FIGS. 577-578, an example clasp 12500 for a device is shown. The clasp 12500 includes a base or fixed arm 12510, a movable arm 12520, and a joint portion 12512. The fixed arm 12510 can be attached to an inner paddle of the device, with the joint portion 12512 disposed proximate the coaptation element. (See, e.g., FIGS. 26-27.) The joint portion 12512 can be spring-loaded so that the fixed and moveable arms 12510, 12520 are biased toward each other when the clasp 12500 is in a closed condition.

Like the various clasps described above, the fixed arm 12510 can be attached to the inner paddle of the device with any suitable means, such as screws or other fasteners, crimped sleeves, mechanical latches or snaps, welding, adhesive, or the like. The fixed arm 12510 remains substantially stationary relative to the inner paddle when the moveable arm 12520 is opened to open the clasp 12500 to capture a leaflet. The clasp 12500 can be opened by applying tension to actuation lines (e.g., the actuation lines 216 shown in FIGS. 43-48) attached to the moveable arms 12520, thereby causing the moveable arms 12520 to pivot or flex on the joint portions 12512.

The clasp 12500 can include a variety of features that increase friction between the fixed and moveable arms 12510, 12520 and the tissue of the leaflet to better retain the leaflet within the clasp. For example, the fixed arm 12510 and the moveable arm 12520 can be formed to have a complementary curved or wavy shape, such as the sinusoidal pattern shown in FIGS. 577-578. The sinusoidal pattern of the fixed arm 12510 and moveable arm 12520 has a peak arranged near the distal ends of the fixed arm 12510 and the moveable arm 12520 and a trough arranged near the joint portion 12512. The curved shape of the fixed arm 12510 and the moveable arm 12520 provides an increased gripping surface between the joint portion 12512 and the distal ends of the fixed arm 12510 and the moveable arm 12520 than a straight configuration.

The clasp 12500 also includes a gripping protrusion 12530 that extends from the moveable arm 12520 and that provides a discrete increase in friction between the clasp 12500 and the leaflet at a predetermined location along the length of the fixed arm 12510 and the moveable arm 12520. The gripping protrusion 12530 is similar to the barbed portions described herein but can have a blunt or rounded end (as shown in FIG. 577) to reduce the potential that gripping the leaflet will cause tissue damage. The gripping protrusion 12530 can be arranged on the moveable arm 12520 as shown or can extend from the fixed arm 12510. Additional gripping protrusions can also be provided at other locations along the fixed arm 12510 and the moveable arm 12520.

A gripping surface 12540 can optionally be included on one or both of the fixed arm 12510 and the moveable arm 12520 as an optional third friction-increasing feature, as shown in FIG. 578. The gripping surface 12540 includes surface features that increase friction retaining force against the leaflet and can take on a wide variety of forms. For example, the gripping surface 12540 can be formed from a plurality of miniature teeth or serrations, such as those shown in FIG. 578. The miniature teeth or serrations can be angled toward an apex at which the clasp and inner paddle intersect to increase retention force against the leaflet. The gripping surface 12540 can also include—in addition to or instead of the teeth or serrations—a coating that increases the friction between the leaflet tissue and the fixed arm 12510 and the moveable arm 12520.

In some implementations of the clasp 12500 that include a plurality of miniature teeth or serrations, the surface features can be formed in a wide variety of ways. Such as, for example, a powder coating process in which a powder from which the surface features will be generated is sprayed on the metallic surfaces while applying high voltage to the surface, thereby creating an electric field in the direction the surface features—e.g., serrations—are facing. Alternatively, a procedure similar to laser printing can also be used to create friction-increasing surface features. In such an example process, a positively charged drum is passed over by a laser that reverses the charges in the areas the laser hits. A roller that includes the particles which will become the surface features is then passed over the drum so that the particles stick to the negatively charged drum. The coated drum can be then passed over the surfaces of the fixed arm 12510 and the moveable arm 12520 to form the friction-increasing surface features of the gripping surface 12540. Powder coating or laser printing processes can be used to provide a desired size and orientation of the surface retention features to increase engagement with the native tissue of the leaflet while also enabling disengagement from the leaflet tissue when desired. As has been described herein, the clasps 12500 can be covered by a cloth to reduce damage to the tissue that may result from direct contact with the metal and also to allow blood flow over the cells of the leaflet tissue through the gaps between the cloth fibers. Thus, the retention features of the gripping surface 12540 are preferably dimensioned to provide increased retention of the leaflet through the cloth covering. Alternatively, the retention features of the gripping surface 12540 can be formed over the cloth covering via the procedures—i.e., laser-printing based—described above.

The retention features of the gripping surface 12540 combined with the curved surfaces of the fixed and the moveable arms 12510, 12520 provide both micro-level and macro-level solutions for increasing the gripping force exerted on the leaflet by the clasp 12500. Because of the increased friction of these other features, the gripping protrusion 12530 can be smaller and/or have a less sharp tip than comparable barbed portions of other clasps so that the gripping protrusion 12530 is less likely to penetrate the native tissue.

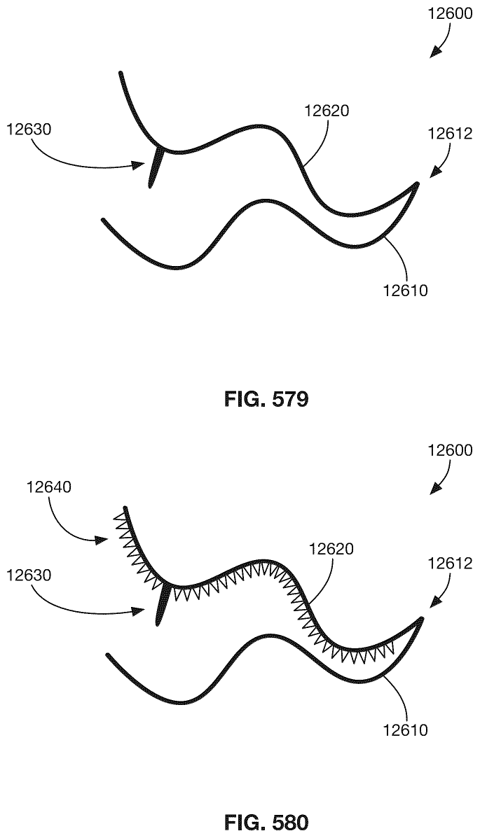

Referring now to FIGS. 579-580, an example clasp 12600 for a device is shown. The clasp 12600 includes a base or fixed arm 12610, a movable arm 12620, and a joint portion 12612. The fixed arm 12610 can be attached to an inner paddle of the device, with the joint portion 12612 disposed proximate the coaptation element. (See, e.g., FIGS. 26-27.) The joint portion 12612 can be spring-loaded so that the fixed and moveable arms 12610, 12620 are biased toward each other when the clasp 12600 is in a closed condition.

Like the various clasps described above, the fixed arm 12610 can be attached to the inner paddle of the device with any suitable means, such as screws or other fasteners, crimped sleeves, mechanical latches or snaps, welding, adhesive, or the like. The fixed arm 12610 remains substantially stationary relative to the inner paddle when the moveable arm 12620 is opened to open the clasp 12600 to capture a leaflet. The clasp 12600 can be opened by applying tension to actuation lines (e.g., the actuation lines 216 shown in FIGS. 43-48) attached to the moveable arms 12620, thereby causing the moveable arms 12620 to pivot or flex on the joint portions 12612.

The clasp 12600 can include a variety of features that increase friction between the fixed and moveable arms 12610, 12620 and the tissue of the leaflet to better retain the leaflet within the clasp. For example, the fixed arm 12610 and the moveable arm 12620 can be formed to have a complementary curved or wavy shape, such as the sinusoidal pattern shown in FIGS. 579-580. The sinusoidal pattern of the fixed arm 12610 and moveable arm 12620 has a peak arranged near a midpoint between the distal ends of the fixed arm 12610 and the moveable arm 12620 and the joint portion 12612 and two troughs arranged on either side of the peak. The curved shape of the fixed arm 12610 and the moveable arm 14520 provides an increased gripping surface between the joint portion 12612 and the distal ends of the fixed arm 12610 and the moveable arm 12620 than a straight configuration.

The clasp 12600 also includes a gripping protrusion 12630 that extends from the moveable arm 12620 and that provides a discrete increase in friction between the clasp 12600 and the leaflet at a predetermined location along the length of the fixed arm 12610 and the moveable arm 12620. The gripping protrusion 12630 is like the barbed portions described herein but can have a blunt or rounded end (as shown in FIG. 579) to reduce the potential that gripping the leaflet will cause tissue damage. The gripping protrusion 12630 can be arranged on the moveable arm 12620 as shown or can extend from the fixed arm 12610. Additional gripping protrusions can also be provided at other locations along the fixed arm 12610 and the moveable arm 12620.

A gripping surface 12640 can optionally be included on one or both of the fixed arm 12610 and the moveable arm 12620 as an optional third friction-increasing feature, as shown in FIG. 580. The gripping surface 12640 includes surface features that increase friction retaining force against the leaflet and can take on a wide variety of forms. For example, the gripping surface 12640 can be formed from a plurality of miniature teeth or serrations, such as those shown in FIG. 576. The miniature teeth or serrations can be angled toward an apex at which the clasp and inner paddle intersect to increase retention force against the leaflet. The gripping surface 12640 can also include—in addition to or instead of the teeth or serrations—a coating that increases the friction between the leaflet tissue and the fixed arm 12610 and the moveable arm 12620.

In some implementations of the clasp 12600 that include a plurality of miniature teeth or serrations, the surface features can be formed in a wide variety of ways. Such as, for example, a powder coating process in which a powder from which the surface features will be generated is sprayed on the metallic surfaces while applying high voltage to the surface, thereby creating an electric field in the direction the surface features—e.g., serrations—are facing. Alternatively, a procedure like laser printing can also be used to create friction-increasing surface features. In such an example process, a positively charged drum is passed over by a laser that reverses the charges in the areas the laser hits. A roller that includes the particles which will become the surface features is then passed over the drum so that the particles stick to the negatively charged drum. The coated drum can be then passed over the surfaces of the fixed arm 12610 and the moveable arm 12620 to form the friction-increasing surface features of the gripping surface 12640. Powder coating or laser printing processes can be used to provide a desired size and orientation of the surface retention features to increase engagement with the native tissue of the leaflet while also enabling disengagement from the leaflet tissue when desired. As has been described herein, the clasps 12600 can be covered by a cloth to reduce damage to the tissue that may result from direct contact with the metal and to allow blood flow over the cells of the leaflet tissue through the gaps between the cloth fibers. Thus, the retention features of the gripping surface 12640 are preferably dimensioned to provide increased retention of the leaflet through the cloth covering. Alternatively, the retention features of the gripping surface 12640 can be formed over the cloth covering via the procedures—i.e., laser-printing based—described above.

The retention features of the gripping surface 12640 combined with the curved surfaces of the fixed and the moveable arms 12610, 12620 provide both micro-level and macro-level solutions for increasing the gripping force exerted on the leaflet by the clasp 12600. Because of the increased friction of these other features, the gripping protrusion 12630 can be smaller and/or have a less sharp tip than comparable barbed portions of other clasps so that the gripping protrusion 12630 is less likely to penetrate the native tissue.

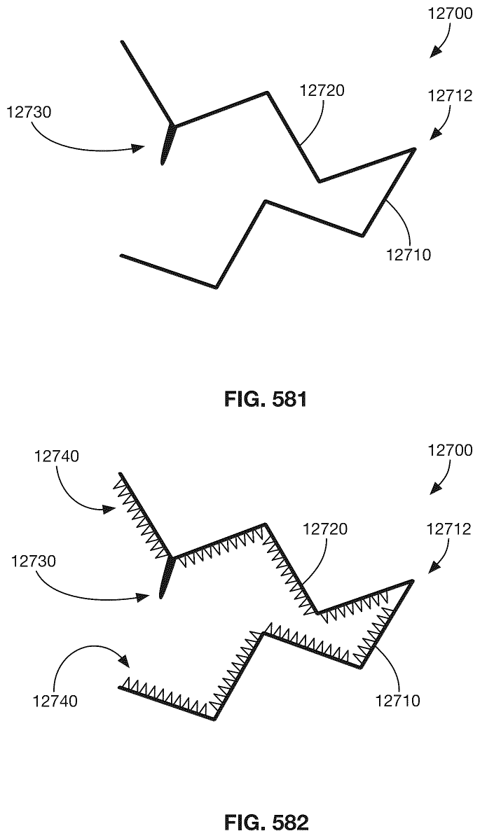

Referring now to FIGS. 581-582, an example clasp 12700 for a device is shown. The clasp 12700 includes a base or fixed arm 12710, a movable arm 12720, and a joint portion 12712. The fixed arm 12710 can be attached to an inner paddle of the device, with the joint portion 12712 disposed proximate the coaptation element. (See, e.g., FIGS. 26-27.) The joint portion 12712 can be spring-loaded so that the fixed and moveable arms 12710, 12720 are biased toward each other when the clasp 12700 is in a closed condition.

Like the various clasps described above, the fixed arm 12710 can be attached to the inner paddle of the device with any suitable means, such as screws or other fasteners, crimped sleeves, mechanical latches or snaps, welding, adhesive, or the like. The fixed arm 12710 remains substantially stationary relative to the inner paddle when the moveable arm 12720 is opened to open the clasp 12700 to capture a leaflet. The clasp 12700 can be opened by applying tension to actuation lines (e.g., the actuation lines 216 shown in FIGS. 43-48) attached to the moveable arms 12720, thereby causing the moveable arms 12720 to pivot or flex on the joint portions 12712.

The clasp 12700 can include a variety of features that increase friction between the fixed and moveable arms 12710, 12720 and the tissue of the leaflet to better retain the leaflet within the clasp. For example, the fixed arm 12710 and the moveable arm 12720 can be formed to have a complementary curved or wavy shape, such as the triangle pattern shown in FIGS. 581-582. The triangle wave pattern of the fixed arm 12710 and moveable arm 12720 has a peak arranged near the joint portion 12712 and a trough arranged near the distal ends of the fixed arm 12710 and the moveable arm 12720. The triangular shape of the fixed arm 12710 and the moveable arm 14520 provides an increased gripping surface between the joint portion 12712 and the distal ends of the fixed arm 12710 and the moveable arm 12720 than a straight configuration. While the curve shown is a triangle wave, various other curves are possible, such as, for example, a saw-tooth wave.

The clasp 12700 also includes a gripping protrusion 12730 that extends from the moveable arm 12720 and that provides a discrete increase in friction between the clasp 12700 and the leaflet at a predetermined location along the length of the fixed arm 12710 and the moveable arm 12720. The gripping protrusion 12730 is like the barbed portions described herein but can have a blunt or rounded end (as shown in FIG. 581) to reduce the potential that gripping the leaflet will cause tissue damage. The gripping protrusion 12730 can be arranged on the moveable arm 12720 as shown or can extend from the fixed arm 12710. Additional gripping protrusions can also be provided at other locations along the fixed arm 12710 and the moveable arm 12720.

A gripping surface 12740 can optionally be included on one or both of the fixed arm 12710 and the moveable arm 12720 as an optional third friction-increasing feature, as shown in FIG. 582. The gripping surface 12740 includes surface features that increase friction retaining force against the leaflet and can take on a wide variety of forms. For example, the gripping surface 12740 can be formed from a plurality of miniature teeth or serrations, such as those shown in FIG. 582. The miniature teeth or serrations can be angled toward an apex at which the clasp and inner paddle intersect to increase retention force against the leaflet. The gripping surface 12740 can also include—in addition to or instead of the teeth or serrations—a coating that increases the friction between the leaflet tissue and the fixed arm 12710 and the moveable arm 12720.

In some implementations of the gripping surface 12740 that include a plurality of miniature teeth or serrations, the surface features can be formed in a wide variety of ways. Such as, for example, a powder coating process in which a powder from which the surface features will be generated is sprayed on the metallic surfaces while applying high voltage to the surface, thereby creating an electric field in the direction the surface features—e.g., serrations—are facing. Alternatively, a procedure like laser printing can also be used to create friction-increasing surface features. In such an example process, a positively charged drum is passed over by a laser that reverses the charges in the areas the laser hits. A roller that includes the particles which will become the surface features is then passed over the drum so that the particles stick to the negatively charged drum. The coated drum can be then passed over the surfaces of the fixed arm 12710 and the moveable arm 12720 to form the friction-increasing surface features of the gripping surface 12740. Powder coating or laser printing processes can be used to provide a desired size and orientation of the surface retention features to increase engagement with the native tissue of the leaflet while also enabling disengagement from the leaflet tissue when desired. As has been described herein, the clasps 12700 can be covered by a cloth to reduce damage to the tissue that may result from direct contact with the metal and to allow blood flow over the cells of the leaflet tissue through the gaps between the cloth fibers. Thus, the retention features of the gripping surface 12740 are preferably dimensioned to provide increased retention of the leaflet through the cloth covering. Alternatively, the retention features of the gripping surface 12740 can be formed over the cloth covering via the procedures—i.e., laser-printing based—described above.

The retention features of the gripping surface 12740 combined with the curved surfaces of the fixed and the moveable arms 12710, 12720 provide both micro-level and macro-level solutions for increasing the gripping force exerted on the leaflet by the clasp 12700. Because of the increased friction of these other features, the gripping protrusion 12730 can be smaller and/or have a less sharp tip than comparable barbed portions of other clasps so that the gripping protrusion 12730 is less likely to penetrate the native tissue.

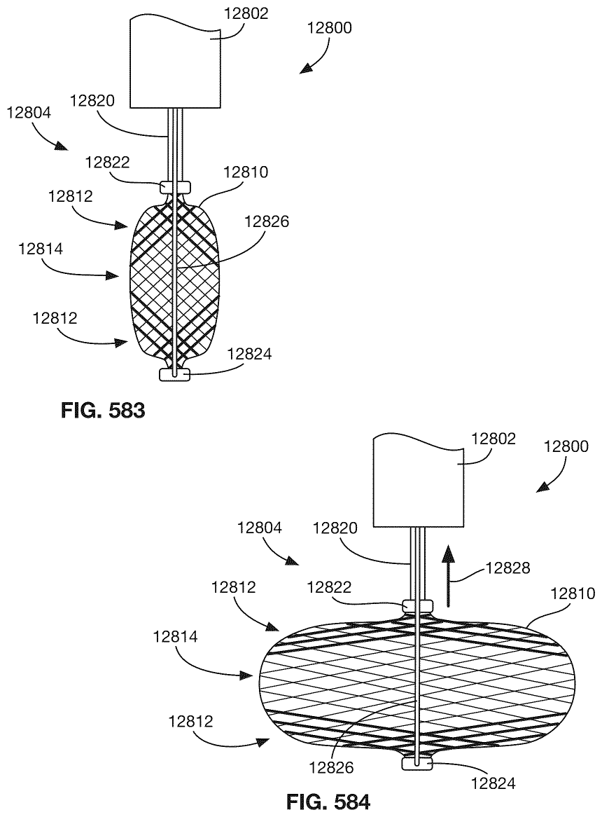

Referring now to FIGS. 583-584, an example of an implantable coaptation device 12800 is shown. The device 12800 can include any other features for a device (e.g., implantable device, repair device, etc.) discussed in the present application, and the device 12800 can be positioned to engage valve tissue as part of any suitable valve repair system (e.g., any valve repair system disclosed in the present application). The coaptation device 12800 can be deployed from a delivery sheath or means for delivery 12802 by a pusher 12820, such as a rod or tube as described above. The device 12800 can include a coaptation portion 12804 and an anchor portion having two more anchors, such as the anchor portion and anchors described herein. The coaptation portion 12804 includes an expandable coaptation element 12810 that can be actuated between a contracted condition (FIG. 583) and an expanded condition (FIG. 584).

The expandable coaptation member or element 12810 is formed from a braided or woven tube of material, such as nitinol wire that extends between the first collar 12822 and the second collar 12824. End portions 12812 of the expandable coaptation member or element 12810 are formed from a braided material having thicker and/or stiffer strands of material and a middle portion 12814 of the expandable coaptation member or element 12810 is formed from thinner and/or more flexible strands of material. Consequently, the middle portion 12814 is more flexible and can expand to a greater degree than the end portions 12812 that are firmer and/or stiff than the middle portion 12814. In some implementations, the expandable coaptation member or element 12810 is formed from a flat sheet or tubing that is laser cut and can be cut so that the middle portion 12814 is more flexible than the end portions 12812. The expandable coaptation member or element 12810 can also expand laterally in a symmetrical fashion (FIG. 584) and/or asymmetrically to accommodate different shaped gaps left between the leaflets 20, 22 during diastole when the native heart valve closes around the device 12800.

The expandable coaptation member or element 12810 extends between a first or proximal collar 12822 and a second or distal collar 12824. Reducing the axial length of the expandable coaptation member or element 12810 between the first collar 12822 and the second collar 12824 causes the expandable coaptation member or element 12810 to expand laterally. Because the middle portion 12814 of the expandable coaptation member or element 12810 is more flexible than the end portions 12812 of the expandable coaptation member or element 12810, the middle portion 12814 expands to a greater degree than the end portions 12812. The flexibility or pliability of the middle portion 12814 can also allow the surface of the expandable coaptation member or element 12810 to conform to the shape of the native leaflets 20, 22 when the leaflets 20, 22 close against the implanted device 12800.

An actuation member 12826 (e.g., wire, rod, shaft, tube, etc.) extends from the delivery sheath or means of delivery 12802 to the second collar 12824. Retracting the actuation member 12826 in a retraction direction 12828 (FIG. 584) moves the second collar 12824 proximally toward the first collar 12822 to reduce the distance between the first collar 12822 and the second collar 12824, thereby causing the expandable coaptation member or element 12810 to expand. The expandable coaptation member or element 12810 can be biased toward the contracted condition or toward the expanded condition and can be actuated between the contracted condition and the expanded condition via extension or retraction of the actuation member 12826.

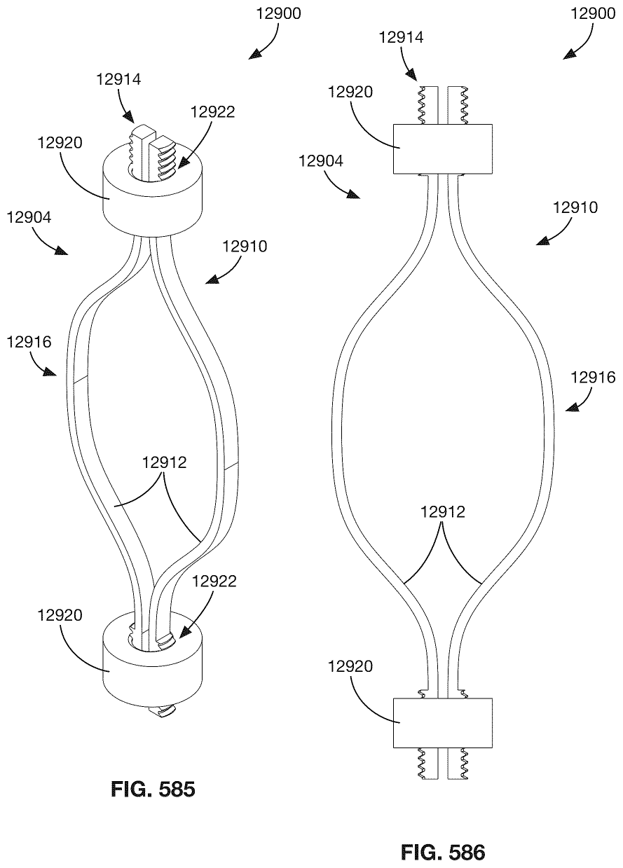

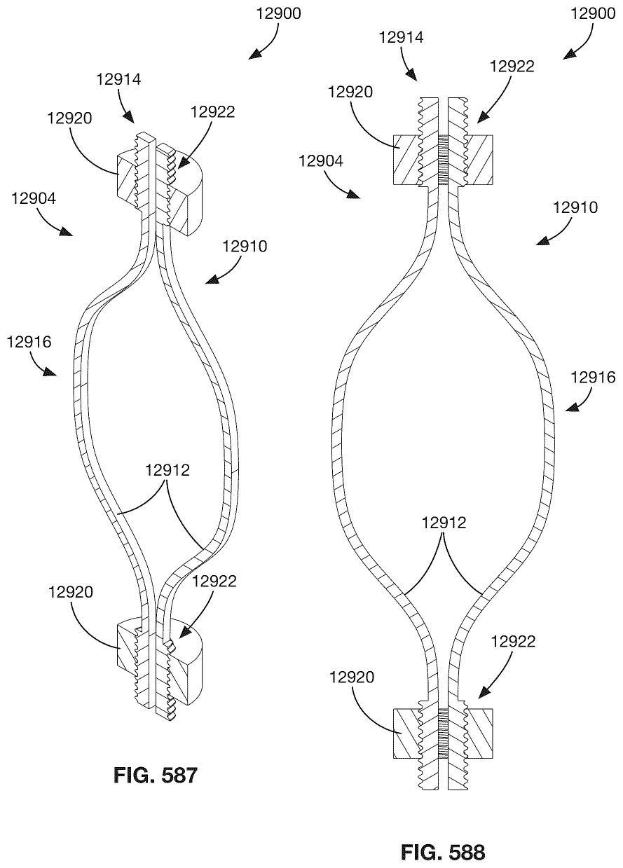

Referring now to FIGS. 585-588, an example expandable coaptation element 12910 (e.g., spacer, coaption element, gap filler, etc.) for use in an implantable coaptation device 12900 is shown. The device 12900 can include any other features for a device (e.g., an implantable device, a repair device, etc.) discussed elsewhere in the present application, and the device 12900 can be positioned to engage valve tissue as part of any suitable valve repair system (e.g., any valve repair system disclosed in the present application). The coaptation device 12900 can be deployed from a delivery sheath or means for delivery by a pusher, such as a rod or tube as described above. The device 12900 can include a coaptation portion 12904 and an anchor portion having two or more anchors, such as the anchor portion and anchors described herein. The coaptation portion 12904 includes an expandable coaptation element 12910 that can be actuated between a contracted condition and an expanded condition (FIGS. 585-588). An optional cover (not shown) can be included to cover a portion or all of the expandable coaptation member or element 12910.

The expandable coaptation member or element 12910 is formed from one or more struts 12912. The struts 12912 extend between threaded ends 12914 and have a flexible middle portion 12916. One or more nuts 12920 having threaded openings 12922 are threaded onto the threaded ends 12914 of the struts 12912. Relative rotation of the nuts 12920 and struts 12912 pulls or flexes the middle portions of the struts toward one another (or allows the middle portions of the struts to relax and move away from one another). That is, tightening (e.g., moving toward the middle portion) the nuts 12920 squeezes the flexible middle portion 12916 together, thereby causing the flexible middle portion 12916 to move laterally inward. The nuts 12920 can also include an engagement portion (not shown) that extends toward and engages the flexible middle portion 12916 to directly move the flexible middle portion 12916 inward as the threaded ends 12914 are moved apart from each other. Rotating the nuts 12920 in the opposite direction causes the threaded ends 12914 to move toward each other to compress the flexible middle portion 12916, thereby causing the flexible middle portion 12916 to move laterally outward. In this way, the expandable coaptation member or element 12910 can be caused to expand and contract laterally to accommodate different size and shape gaps left between the leaflets 20, 22 during diastole when the native heart valve closes around the device 12900.

The flexibility or pliability of the flexible middle portion 12916 can also allow the surface of the expandable coaptation member or element 12910 (and also an optional cover) to conform to the shape of the native leaflets 20, 22 when the leaflets 20, 22 close against the implanted device 12900. The struts can be formed from a shape-set material and the flexible middle portion 12916 of the struts 12912 can be shape set to a wide variety of shapes to improve engagement between the struts 12912 and the native leaflets 20, 22. While two struts 12912 are shown, any number of struts 12912 can be combined to provide expanding and contracting coaptation surfaces. Different struts can include different flexible middle portions 12916 to provide differing rates of expansion and contraction and different maximum expansion positions to form a wide variety of differently shaped expandable coaptation members or elements 12910.

The nuts 12920 can be rotated simultaneously or separately and the threaded openings 12922 can have left-handed or right-handed threads. Alternatively, a single nut 12920 can be used to cause the flexible middle portion 12916 to expand and contract. That is, one nut 12920 can be rotated while the other remains stationary, or the expandable coaptation member or element 12910 can include only one nut 12920 while one end of the struts 12912 remains fixed. The expandable coaptation member or element 12910 can be biased toward the contracted condition or toward the expanded condition and can be actuated between the contracted condition and the expanded condition via adjustment of the one or more nuts 12920.

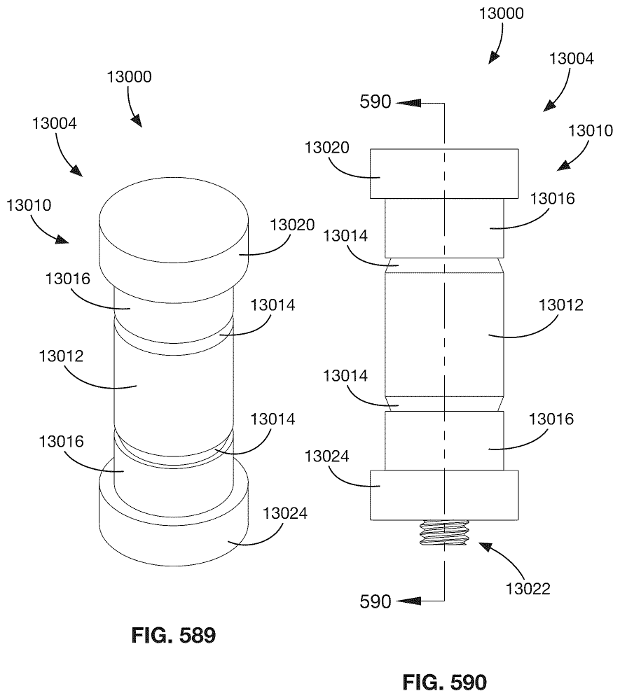

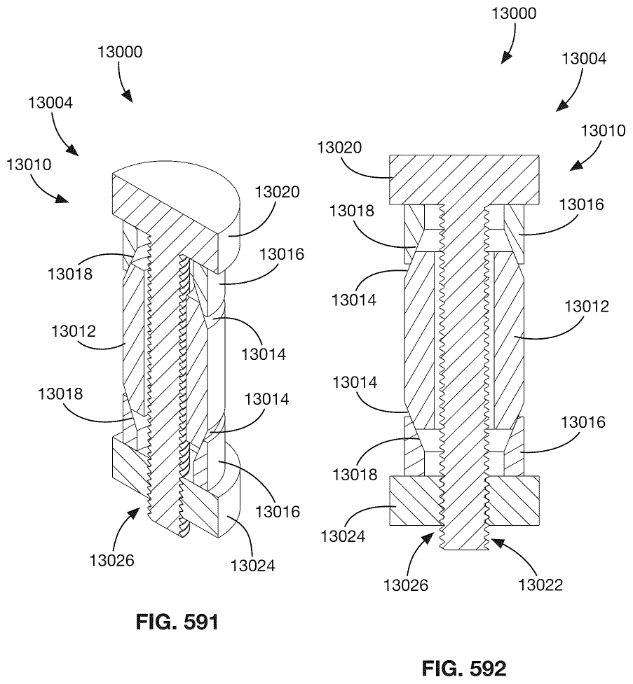

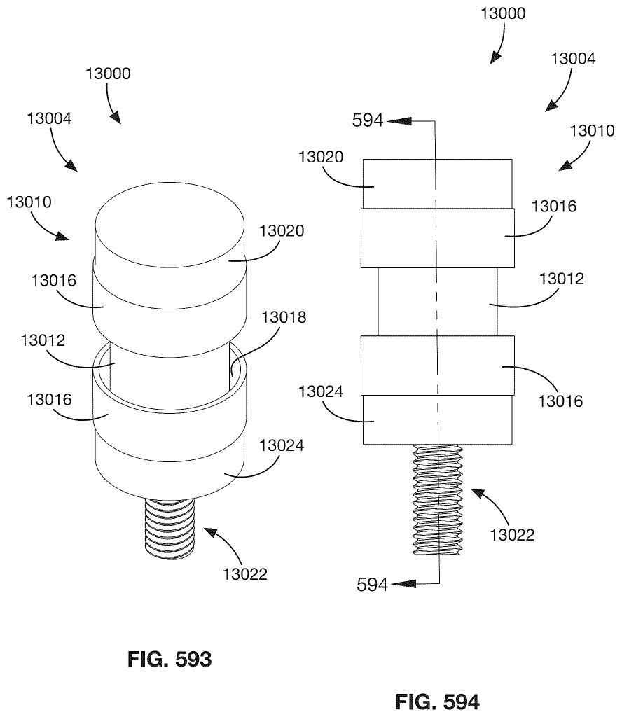

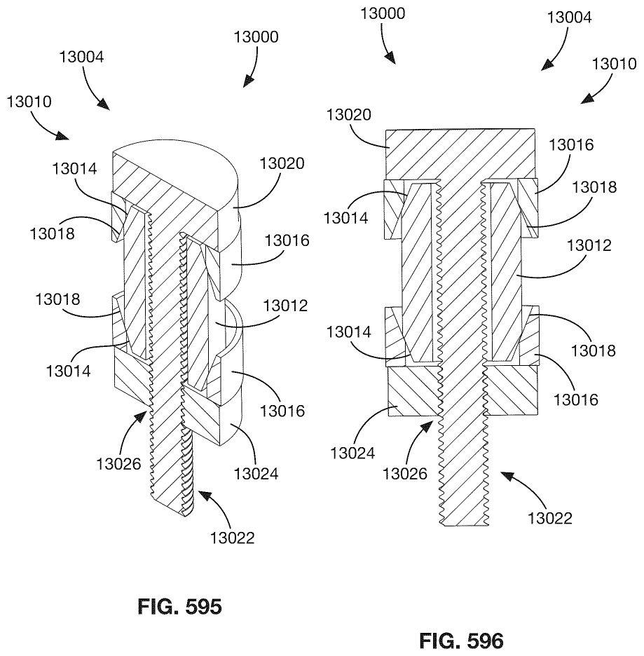

Referring now to FIGS. 589-596, an example expandable coaptation element 13010 (e.g., spacer, coaption element, gap filler, etc.) for use in a device 13000 (e.g., an implantable device, a spacer device, a valve repair device, etc.) is shown. The device 13000 can include any other features for a device discussed elsewhere in the present application, and the device 13000 can be positioned to engage valve tissue as part of any suitable valve repair system (e.g., any valve repair system disclosed in the present application). The coaptation device 13000 can be deployed from a delivery sheath or means for delivery by a pusher, such as a rod or tube as described above. The device 13000 can include a coaptation portion 13004 and an anchor portion having two or more anchors, such as the anchor portion and anchors described herein. The expandable coaptation portion 13004 can be actuated between a contracted condition (FIGS. 589-592) and an expanded condition (FIGS. 593-596). An optional cover (not shown) can be included to cover a portion or all of the expandable coaptation member or element 13010.

The expandable coaptation member or element 13010 is formed from a fixed member 13012 (e.g., tube or shaft with tapered ends, etc.) and one or more moveable or expansion members 13014 (e.g., tube or shaft with tapered ends, etc.). The fixed member 13012 has a cylindrical shape with outward facing ramp or incline portions 13016 at each end. The expansion members 13014 have an annular or ring shape with an internal ramp or incline portion 13018. The internal ramp portions 13018 of the expansion members 13014 engage the external ramp portions 13016 of the fixed member 13012. A first or proximal actuation member 13020 (e.g., wire, rod, tube, shaft, etc.) includes a distally extending threaded shaft 13022 that extends through the fixed member 13012 and the one or more expansion members 13014. A second or distal actuation member 13024 (e.g., collar, nut, etc.) includes a threaded opening 13026 for engaging the threaded shaft 13022 of the first actuation member 13020.

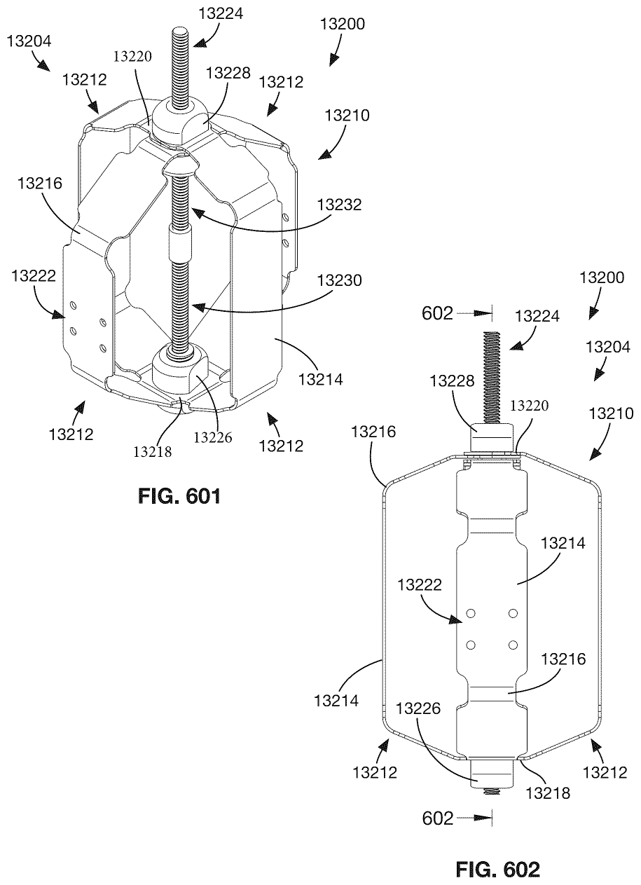

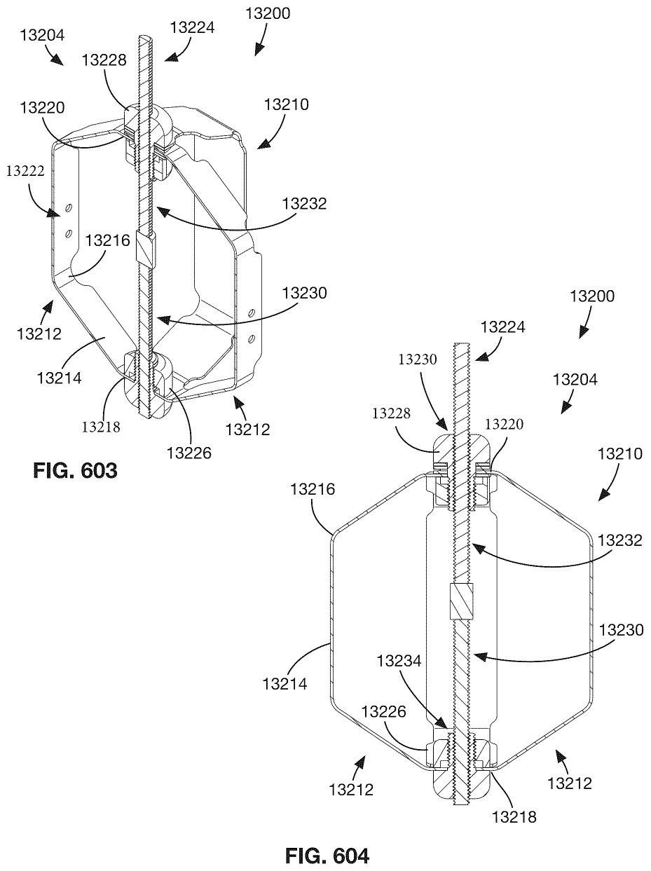

Relative rotation of the first actuation member 13020 and second actuation member 13024 moves the second actuation member 13024 along the threaded shaft 13022 toward or away from the first actuation member 13020 along the threaded shaft 13022. In particular, rotating the first actuation member 13020 while prohibiting rotation of the second actuation member 13024—or vice versa-causes the second actuation member 13024 to move along the threaded shaft 13022. Alternatively, the first actuation member 13020 and the second actuation member 13024 can both have threaded openings that engage a threaded shaft with two different thread portions, each thread portion having threads in a different direction. (An actuation shaft with two different thread portions can be like the actuation shaft 13124 shown in FIGS. 601-604 and described in greater detail below.) For example, the first actuation member 13020 and a first portion of the threaded shaft can have threads in a right-handed direction while the second actuation member 13024 and a second portion of the threaded shaft can have threads in a left-handed direction. Thus, rotating the threaded shaft in one direction moves the first actuation member 13020 and the second actuation member 13024 in opposite directions. This arrangement has an added benefit that a midplane formed between the expansion members 13014 does not move as both the first actuation member 13020 and the second actuation member 13024 are moved in opposing directions along the threaded shaft.

As the first actuation member 13020 and the second actuation member 13024 are moved toward each other by rotation of the threaded shaft 13022, each of the first actuation member 13020 and the second actuation member 13024 engage one of the expansion members 13014 to move the expansion members 13014 toward each other, as can be seen in FIGS. 593-596. Moving the expansion members 13014 closer together causes the internal ramp portions 13018 of the expansion members 13014 to engage the external ramp portions 13016 of the fixed member 13012. The interaction of the internal ramp portions 13018 and the external ramp portions 13016 transmit the force generated by movement of the first actuation member 13020 and the second actuation member 13024 into a radially directed expansion force that causes the expansion members 13014 to expand outward. The expansion members are formed from a flexible material so that the radial expansion force causes the expansion members 13014 to stretch around the increasing diameter of the fixed member 13012 as the expansion members 13014 are moved along the external ramp portions 13016. In this way, the relative rotation of the first actuation member 13020 and the second actuation member 13024 causes the outer diameter of the expandable coaptation member or element 13010 to increase and decrease to accommodate different size and shape gaps left between the leaflets 20, 22 during diastole when the native heart valve closes around the device 13000.

The flexibility or pliability of the expansion members 13014 can also allow the surface of the expandable coaptation member or element 13010 (and also an optional cover) to conform to the shape of the native leaflets 20, 22 when the leaflets 20, 22 close against the implanted device 13000. The diameter of the fixed member 13012 and the thickness of the expansion members 13014 can be varied to alter the maximum outer diameter of the expandable coaptation member or element 13010. The incline of the external ramp portions 13016 and the internal ramp portions 13018 can also be varied to change the rate of expansion of the expandable coaptation member or element 13010 as the first actuation member 13020 and the second actuation member 13024 are rotated relative to each other. While two expansion members 13014 are shown, any number of expansion members 13014 can be combined to provide expanding and contracting coaptation surfaces. Different expansion members 13014 can include different thicknesses or ramp slopes to provide differing rates of expansion and contraction and different maximum expansion positions to form a wide variety of differently shaped expandable coaptation members or elements 13010.

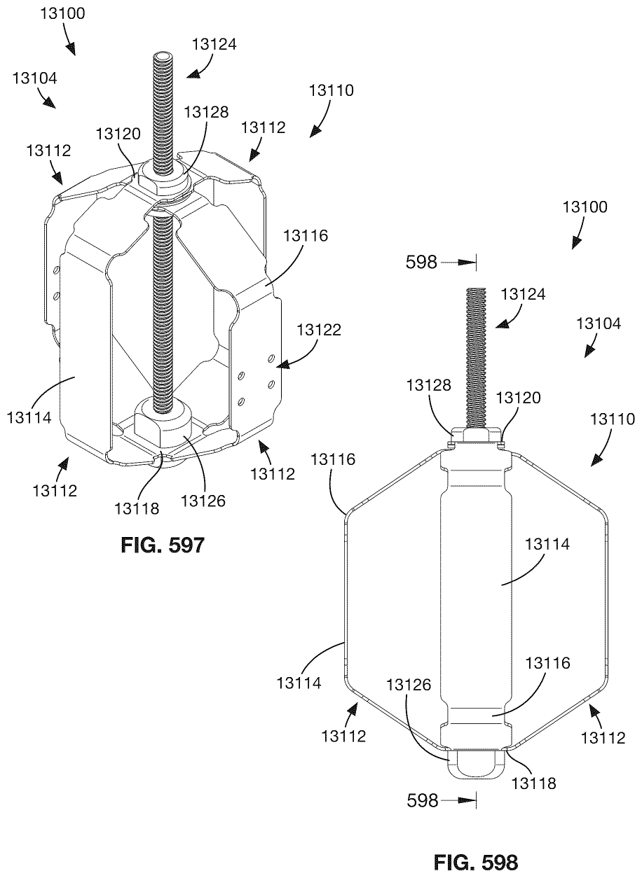

Referring now to FIGS. 597-600, an example expandable coaptation element 13110 (e.g., spacer, coaption element, gap filler, etc.) for use in a device 13100 (e.g., an implantable device, a spacer device, a valve repair device, etc.) is shown. The device 13100 can include any other features of devices discussed elsewhere in the present application, and the device 13100 can be positioned to engage valve tissue as part of any suitable valve repair system (e.g., any valve repair system disclosed in the present application). The coaptation device 13100 can be deployed from a delivery sheath or means for delivery by a pusher, such as a rod or tube as described above. The device 13100 can include a coaptation portion 13104 and an anchor portion having two or more anchors, such as the anchor portion and anchors described herein. The coaptation portion 13104 includes an expandable coaptation element 13110 that can be actuated between a contracted condition and an expanded condition (FIGS. 597-600). An optional cover (not shown) can be included to cover a portion or all of the expandable coaptation member or element 13110.

The expandable coaptation member or element 13110 is formed from one or more struts 13112. The struts 13112 have a plurality of rigid portions 13114 that are connected together by hinge portions 13116 that enable the rigid portions 13114 to bend relative to each other. A fixed end portion 13118 is connected to each of the struts 13112 by the hinge portions 13116. The struts 13112 extend from the fixed end portion 13118 to a moveable end portion 13120 connected to each strut 13112 by one of the hinge portions 13116. One or more of the rigid portions 13114 can include an attachment region 13122 for attaching another component of the expandable coaptation member or element 13110 or of the coaptation device 13100.

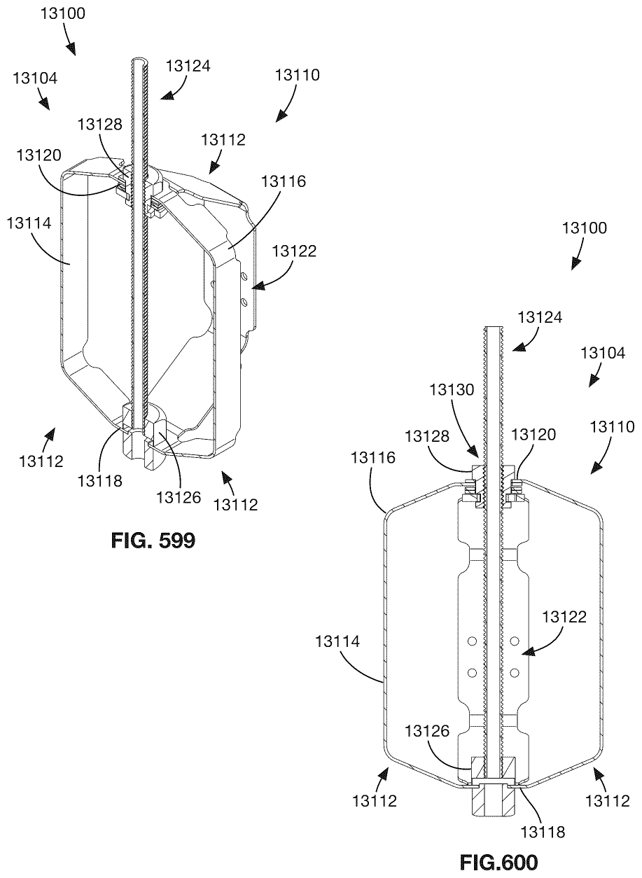

The struts 13112 are moved between a collapsed condition and an expanded condition by the actuation of a threaded actuation shaft 13124 that extends through the expandable coaptation member or element 13110 to a fixed actuation member 13126. The fixed end portion 13118 is attached to the fixed actuation member 13126 and the moveable end portions 13120 are attached to the moveable actuation member 13128. The threaded actuation shaft 13126 extends proximally from the fixed actuation member 13126 and through a moveable actuation member 13128. The threaded actuation shaft 13124 can be hollow and the fixed actuation member 13126 and the moveable actuation member 13128, as shown in FIGS. 599-600, so that other actuation members and other components of the device can extend through the expandable coaptation member or element 13110 and can be moved or actuated independently of the actuation of the expandable coaptation member or element 13110.

The moveable actuation member 13128 includes a threaded opening 13130 that engages the threads of the threaded actuation shaft 13124 so that rotation of the threaded actuation shaft 13124 relative to the moveable actuation member 13128—or vice versa-moves the moveable actuation member 13128 toward and away from the fixed actuation member 13126. That is, rotating the moveable actuation member 13128 via an actuation shaft or other mechanism (not shown) causes the moveable actuation member 13128 to move proximally away from the fixed actuation member 13126 and distally toward the fixed actuation member 13126. As the moveable actuation member 13128 is moved along the threaded actuation shaft 13124, the struts 13112 expand and contract to change the overall width of the expandable coaptation member or element 13110. The expandable coaptation member or element 13110 has a minimum width when the struts 13112 are stretched to an almost nearly or straight condition and/or when the fixed end portion 13118 and the moveable end portion 13120 are moved close to each other so that the rigid portions 13114 are folded against each other. In this way, the expandable coaptation member or element 13110 can be caused to expand and contract laterally to accommodate different size and shape gaps left between the leaflets 20, 22 during diastole when the native heart valve closes around the device 13100.

The rigid portions 13114 of the struts 13112 and also an optional cover covering the struts 13112 can include shaped portions or can include flexible or pliable portions to allow the surface of the expandable coaptation member or element 13110 to conform to the shape of the native leaflets 20, 22 when the leaflets 20, 22 close against the implanted device 13100 to improve engagement between the struts 13112 and the native leaflets 20, 22. While four struts 13112 are shown, any number of struts 13112 can be combined to provide expanding and contracting coaptation surfaces. Different struts can include rigid portions 13114 having different shapes and lengths to provide differing rates of expansion and contraction and different maximum expansion positions to form a wide variety of differently shaped expandable coaptation members or elements 13110.

Referring now to FIGS. 601-604, an example expandable coaptation element 13210 for use in a device 13200 (e.g., an implantable device, a spacer device, a valve repair device, etc.) is shown. The device 13200 can include any other features of devices discussed elsewhere in the present application, and the device 13200 can be positioned to engage valve tissue as part of any suitable valve repair system (e.g., any valve repair system disclosed in the present application). The coaptation device 13200 can be deployed from a delivery sheath or means for delivery by a pusher, such as a rod or tube as described above. The device 13200 can include a coaptation portion 13204 and an anchor portion having two or more anchors, such as the anchor portion and anchors described herein. The coaptation portion 13204 includes an expandable coaptation element 13210 that can be actuated between a contracted condition and an expanded condition (FIGS. 601-604). An optional cover (not shown) can be included to cover a portion or all of the expandable coaptation member or element 13210.

The expandable coaptation member or element 13210 is formed from one or more struts 13212. The struts 13212 have a plurality of rigid portions 13214 that are connected together by hinge portions 13216 that enable the rigid portions 13214 to pivot relative to each other. A first end portion 13218 is connected to each of the struts 13212 by the hinge portions 13216. The struts 13212 extend from the first end portion 13218 to a second end portion 13220 connected to each strut 13212 by one of the hinge portions 13216. One or more of the rigid portions 13214 can include an attachment region 13222 for attaching another component of the expandable coaptation member or element 13210 or of the coaptation device 13200.

The struts 13212 are moved between a collapsed condition and an expanded condition by the actuation of a threaded actuation shaft 13224 that extends through a first actuation member 13226 and a second actuation member 13228 of the expandable coaptation member or element 13210. The first end portion 13218 of the struts 13212 is attached to the first actuation member and the second end portions 13220 are attached to the second actuation member 13228. The threaded actuation shaft 13224, the first actuation member 13226, and the second actuation member 13228 can be hollow so that other actuation members and other components of the device can extend through the expandable coaptation member or element 13210 and can be moved or actuated independently of the actuation of the expandable coaptation member or element 13210.

The threaded actuation shaft 13224 includes a first threaded portion 13230 extending from a distal end to a second threaded portion 13232 and the second threaded portion 13232 extends from the first threaded portion 13230 to a proximal end. The threads of the first threaded portion 13230 and the second threaded portion 13232 are oriented in opposite directions; i.e., if the threads of the first threaded portion 13230 are right-handed threads, then the threads of the second threaded portion 13232 are left-handed threads, and vice versa. The first actuation member 13226 engages the first threaded portion 13230 and includes a threaded opening 13234 that has threads that match the orientation of the threads of the first threaded portion 13230. The second actuation member 13228 engages the second threaded portion 13232 and includes a threaded opening 13234 that has threads that match the orientation of the threads of the second threaded portion 13232. Consequently, rotating the threaded actuation shaft 13224 in one direction moves the first actuation member 13226 and the second actuation member 13228 in opposite directions. Because the first actuation member 13226 and the second actuation member 13228 move in opposite directions at the same rate when the threaded actuation shaft 13224 is rotated, a midplane formed between the first actuation member 13226 and the second actuation member 13228 does not move as the expandable coaptation member or element 13210 is expanded and contracted.

During operation of the expandable coaptation member or element 13210, for example, rotating the threaded actuation shaft 13224 can cause the first actuation member 13226 to move in a proximal direction and the second actuation member 13228 to move in a distal direction, thereby decreasing the distance between the first actuation member 13226 and the second actuation member 13228. In the same example, rotating the threaded actuation shaft 13224 in the opposite direction would cause the first actuation member 13226 to move in a distal direction and the second actuation member 13228 to move in a proximal direction, thereby increasing the distance between the first actuation member 13226 and the second actuation member 13228. In this way, rotation of the threaded actuation shaft 13224 is used to expand and contract the struts 13212 of the expandable coaptation member or element 13210. In this way, rotation of the threaded actuation shaft 13224 causes the outer perimeter or size of the expandable coaptation member or element 13210 to increase and decrease to accommodate different size and shape gaps left between the leaflets 20, 22 during diastole when the native heart valve closes around the device 13200.

The rigid portions 13214 of the struts 13212 and also an optional cover covering the struts 13212 can include shaped portions or can include flexible or pliable portions to allow the surface of the expandable coaptation member or element 13210 to conform to the shape of the native leaflets 20, 22 when the leaflets 20, 22 close against the implanted device 13200 to improve engagement between the struts 13212 and the native leaflets 20, 22. While four struts 13212 are shown, any number of struts 13212 can be combined to provided expanding and contracting coaptation surfaces. Different struts can include rigid portions 13214 having different shapes and lengths to provide differing rates of expansion and contraction and different maximum expansion positions to form a wide variety of differently shaped expandable coaptation members or elements 13210.

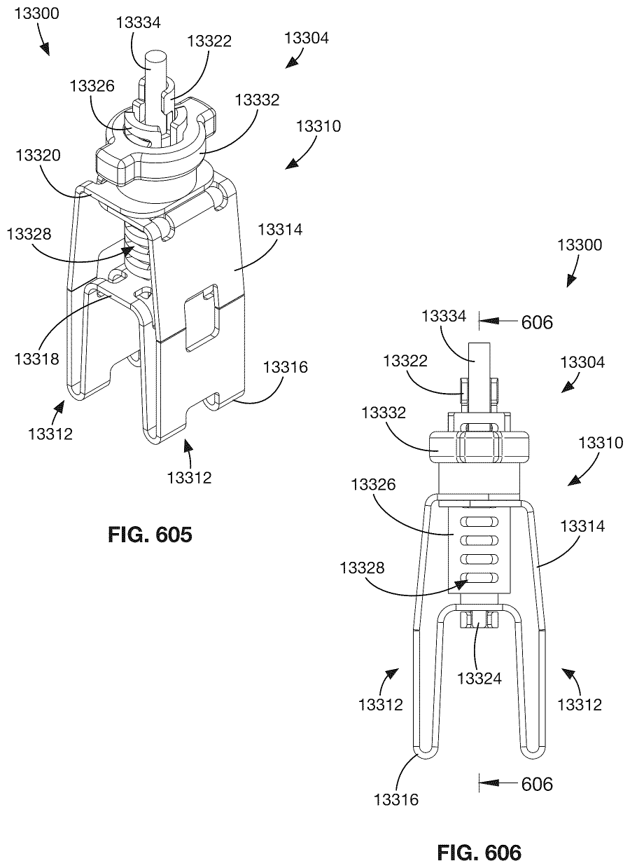

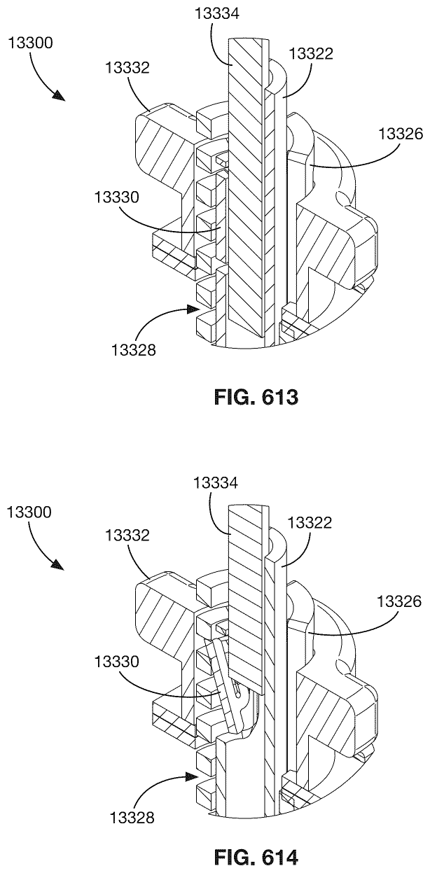

Referring now to FIGS. 605-614, an example expandable coaptation element 13310 (e.g., spacer, coaption element, gap filler, etc.) for use in a device 13300 (e.g., an implantable device, a spacer device, a valve repair device, etc.) is shown. The device 13300 can include any other features of devices discussed elsewhere in the present application, and the device 13300 can be positioned to engage valve tissue as part of any suitable valve repair system (e.g., any valve repair system disclosed in the present application). The coaptation device 13300 can be deployed from a delivery sheath or means for delivery by a pusher, such as a rod or tube as described above. The device 13300 can include a coaptation portion 13304 and an anchor portion having two or more anchors, such as the anchor portion and anchors described herein. In some implementations, the coaptation portion 13304 includes a coaptation element 13310 (e.g., spacer, coaption element, gap filler, etc.) that can be actuated between a contracted condition (FIGS. 605-614) and an expanded condition. An optional cover (not shown) can be included to cover a portion or all of the expandable coaptation member or element 13310.

The expandable coaptation member or element 13310 is formed from one or more struts 13312. The struts 13312 have a plurality of rigid portions 13314 that are connected together by hinge portions 13316 that enable the rigid portions 13314 to pivot relative to each other. A first end portion 13318 is connected to each of the struts 13312 by the hinge portions 13316. The struts 13312 extend from the first end portion 13318 to a second end portion 13320 connected to each strut 13312 by one of the hinge portions 13316. One or more of the rigid portions 13314 can include an attachment region for attaching another component of the expandable coaptation member or element 13310 or of the coaptation device 13300.

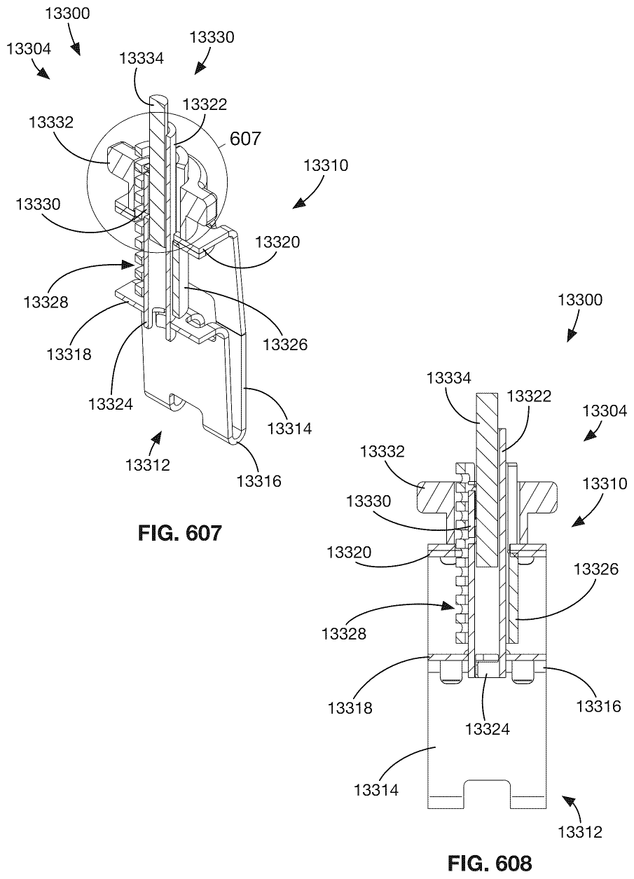

The struts 13312 are moved between a collapsed condition and an expanded condition by the extension and retraction of an actuation tube 13322. The actuation tube 13322 extends to a distal end 13324 that is attached to the first end portion 13318 and moves within a latch tube 13326 that includes a plurality of openings 13328 for engaging with a latch member 13330 (e.g., arm, prong, etc.) of the actuation tube 13322. A retention or securing member 13334 (e.g., wire, rod, shaft, tube, etc.) secures the connection between the actuation tube 13322 and latch tube 13326 by engaging the latch member 13330; the relationship between the actuation tube 13322, the latch tube 13326, the latch member 13330, and the securing member 13334 is shown in FIGS. 607-608 and 611-614 and is described below.

The latch tube 13326 is attached to a proximal collar or head 13332 of the device 13300 that can be coupled to a delivery mechanism for delivering the device 13300 within the native valve. The proximal collar or head 13332 is also attached to the second ends 13320 of the struts 13312. Consequently, extension and retraction of the actuation tube 13322 changes the distance between the first end portion 13318 and the second end portion 13320 of the struts 13312 to cause the struts 13312 to move between the contracted and expanded conditions. The proximal collar or head 13332 can be engaged and actuated in a wide variety of ways described herein (such as, for example, via the mechanisms shown in FIGS. 417-482) to facilitate the implantation of the devices described herein, such as, for example, by opening and closing paddles of the device.

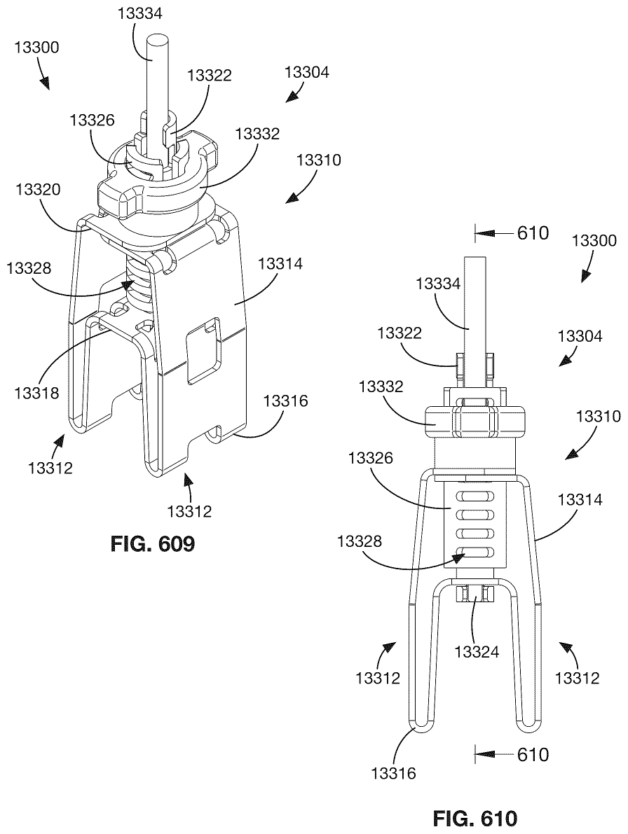

The latch member 13330 of the actuation tube 13322 can be moved between a latched and an unlatched condition. The actuation tube 13322 can include any number of latch members 13330 suitable for engaging corresponding openings 13328 in the latch tube 13326. Referring now to FIGS. 605-608 and 613, the latch member 13330 of the actuation tube 13322 is shown in an unlatched condition. When the latch member 13330 is in the unlatched condition, the actuation tube 13322 can be moved freely along the length of the latch tube 13326 to extend and retract the first end portion 13318, thereby expanding and contracting the expandable coaptation member or element 13310. In the latched condition, shown in FIGS. 609-612 and 614, the latch member 13330 in the latched condition engages the openings 13328 of the latch tube 13326 to secure the position of the actuation tube 13322 relative to the latch tube 13326.

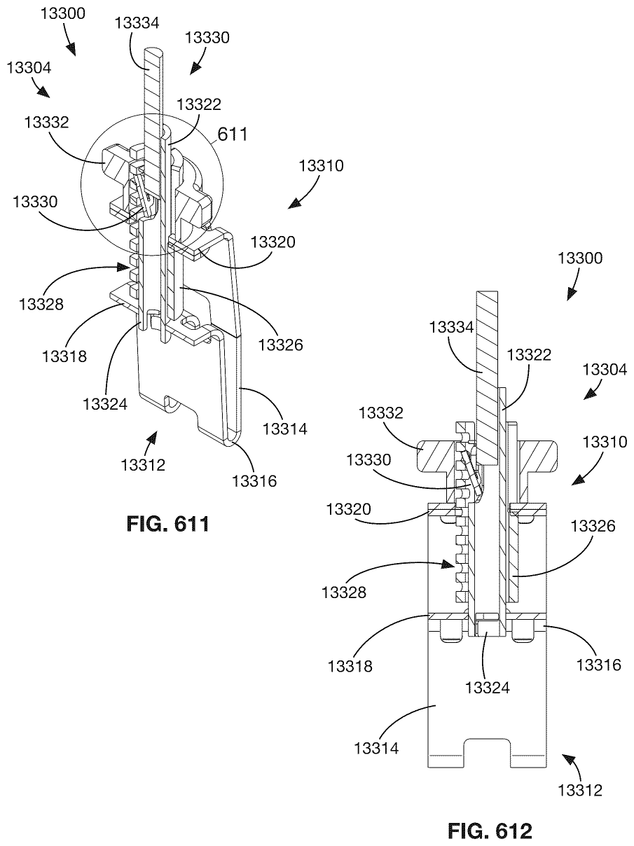

The latch member 13330 can optionally be integrally formed in the side of the actuation tube 13322 by laser-cutting a portion of the actuation tube 13322. The latch member 13330 is bent and shape-set in the latched condition so that in a free state the latch member 13330 is angled relative to a central axis of the actuation tube 13322. For example, as it shown in FIGS. 611-612, the proximal or upper end of the latch member 13330 is biased radially outward and the opposite lower or distal end of the latch member 13330 is biased radially inward. Alternatively, the proximal end of the latch member 13330 can be biased radially inward and the opposite distal end of the latch member 13330 can be biased radially outward. In some implementations, the expandable coaptation member or element 13310 is biased to expand or contract and the latch member 13330 is angled outward to oppose the bias of the expandable coaptation member or element 13310. For example, in an example where the expandable coaptation member or element 13310 is biased to cause the actuation tube 13322 to move in the proximal direction, the proximal end of the latch member 13330 can be biased radially outward so that the latch member 13330 engages the openings 13328 of the latch tube 13326 such that the proximal movement of the actuation tube 13322 is resisted by the latch member 13330.

The latch member 13330 is retained in the unlatched condition by the retaining or securing member 13334 that prohibits the latch member 13330 from pivoting into the latched condition. During implantation of the device 13300 the width of the expandable coaptation member or element 13310 is adjusted by extending and retracting the actuation tube 13322. When the desired width of the expandable coaptation member or element 13310 has been reached, the retention securing member 13334 is retracted from the actuation tube 13322 to allow the latch member 13330 to pivot into the latched condition to engage the openings 13328 of the latch tube 13326. The extension and retraction of the actuation tube 13322 causes the outer diameter of the expandable coaptation member or element 13310 to increase and decrease to accommodate different size and shape gaps left between the leaflets 20, 22 during diastole when the native heart valve closes around the device 13300. Locking the actuation tube 13322 in place relative to the latch tube 13326 via the latch member 13330 maintains the width of the expandable coaptation member or element 13310 against the force applied by the leaflets 20, 22. The retention or securing member 13334 can optionally have a tapered distal end to facilitate re-engagement of the latch member 13330 to cause the latch member 13330 to pivot from the latched condition to the unlatched condition, thereby enabling further adjustment of the width of the expandable coaptation member or element 13310.

The rigid portions 13314 of the struts 13312 and also an optional cover covering the struts 13312 can include shaped portions or can include flexible or pliable portions to allow the surface of the expandable coaptation member or element 13310 to conform to the shape of the native leaflets 20, 22 when the leaflets 20, 22 close against the implanted device 13300 to improve engagement between the struts 13312 and the native leaflets 20, 22. While four struts 13312 are shown, any number of struts 13312 can be combined to provided expanding and contracting coaptation surfaces. Different struts can include rigid portions 13314 having different shapes and lengths to provide differing rates of expansion and contraction and different maximum expansion positions to form a wide variety of differently shaped expandable coaptation members or elements 13310.

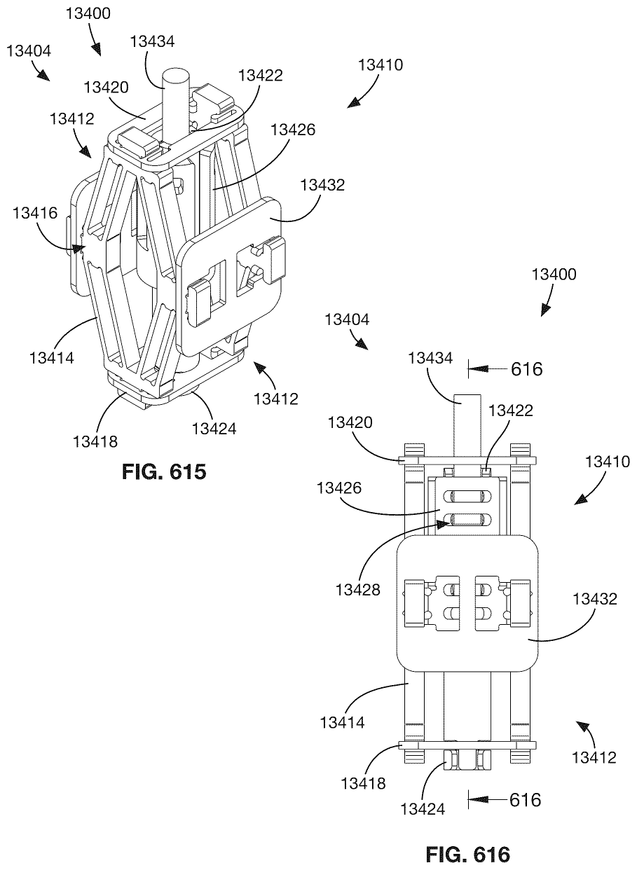

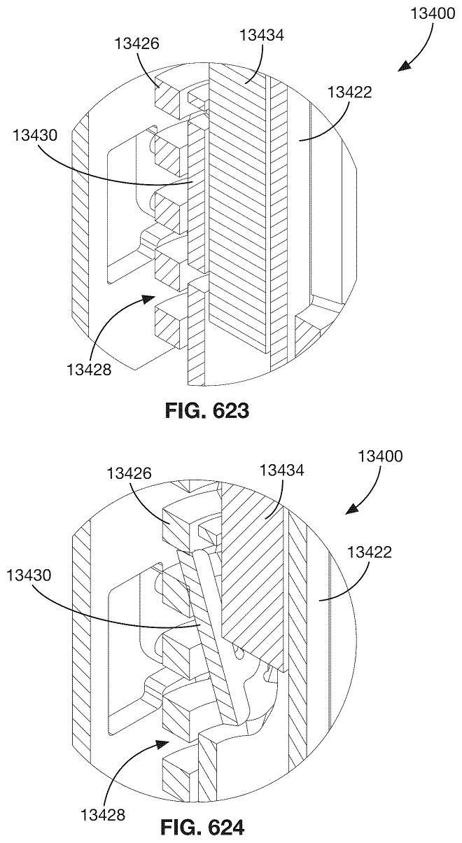

Referring now to FIGS. 615-624, an example expandable coaptation element 13410 (e.g., spacer, coaption element, gap filler, etc.) for use in a device 13400 (e.g., an implantable device, a spacer device, a valve repair device, etc.) is shown. The device 13400 can include any other features of devices discussed elsewhere in the present application, and the device 13400 can be positioned to engage valve tissue as part of any suitable valve repair system (e.g., any valve repair system disclosed in the present application). The coaptation device 13400 can be deployed from a delivery sheath or means for delivery by a pusher, such as a rod or tube as described above. The device 13400 can include a coaptation portion 13404 and an anchor portion having two more anchors, such as the anchor portion and anchors described herein. The coaptation portion 13404 includes an expandable coaptation element 13410 that can be actuated between a contracted condition (FIGS. 615-624) and an expanded condition. An optional cover (not shown) can be included to cover a portion or all of the expandable coaptation member or element 13410.

The expandable coaptation member or element 13410 is formed from one or more struts 13412. The struts 13412 have a plurality of rigid portions 13414 that are connected together by hinge portions 13416 that enable the rigid portions 13414 to bend relative to each other. A first end portion 13418 is connected to each of the struts 13412 by the hinge portions 13416. The struts 13412 extend from the first end portion 13418 to a second end portion 13420 connected to each strut 13412 by one of the hinge portions 13416. Optional side plates 13432 can be attached to the hinge portions 13416 of adjacent struts 13412 to stabilize the struts 13412. One or more of the rigid portions 13414 can include an attachment region, such as, for example, at the side plates 13432, for attaching another component of the expandable coaptation member or element 13410 or of the coaptation device 13400.

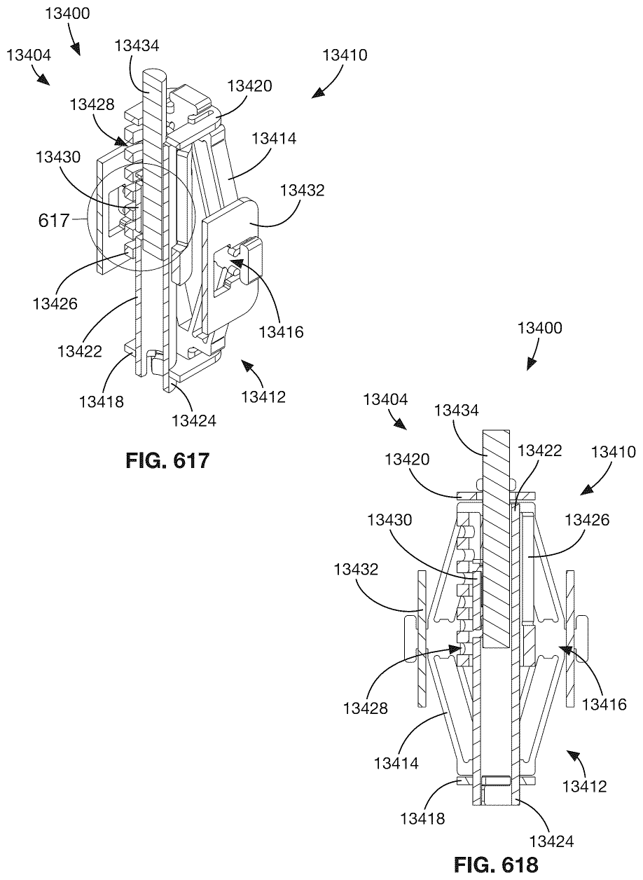

The struts 13412 are moved between a collapsed condition and an expanded condition by the extension and retraction of an actuation tube 13422. The actuation tube 13422 extends to a distal end 13424 that is attached to the first end portion 13418 and moves within a latch tube 13426 that includes a plurality of openings 13428 for engaging with a latch member 13430 of the actuation tube 13422. A width adjustment, such as the illustrated retention or securing member 13434 secures the connection between the actuation tube 13422 and latch tube 13426 by engaging the latch member 13430; the relationship between the actuation tube 13422, the latch tube 13426, the latch member 13430, and the securing member 13434 is shown in FIGS. 617-618 and 621-624 and is described below.

The latch tube 13426 can be attached to a proximal collar or head (not shown) of the device 13400 (e.g., an implantable device, a spacer device, a valve repair device, etc.) that can be coupled to a delivery mechanism for delivering the device 13400 within the native valve. The proximal collar can also be attached to the second ends 13420 of the struts 13412. Consequently, extension and retraction of the actuation tube 13422 changes the distance between the first end portion 13418 and the second end portion 13420 of the struts 13412 to cause the struts 13412 to move between the contracted and expanded conditions. The proximal collar or head can be engaged and actuated in a wide variety of ways described herein (such as, for example, via the mechanisms shown in FIGS. 417-482) to facilitate the implantation of the devices described herein, such as, for example, by opening and closing paddles of the device.

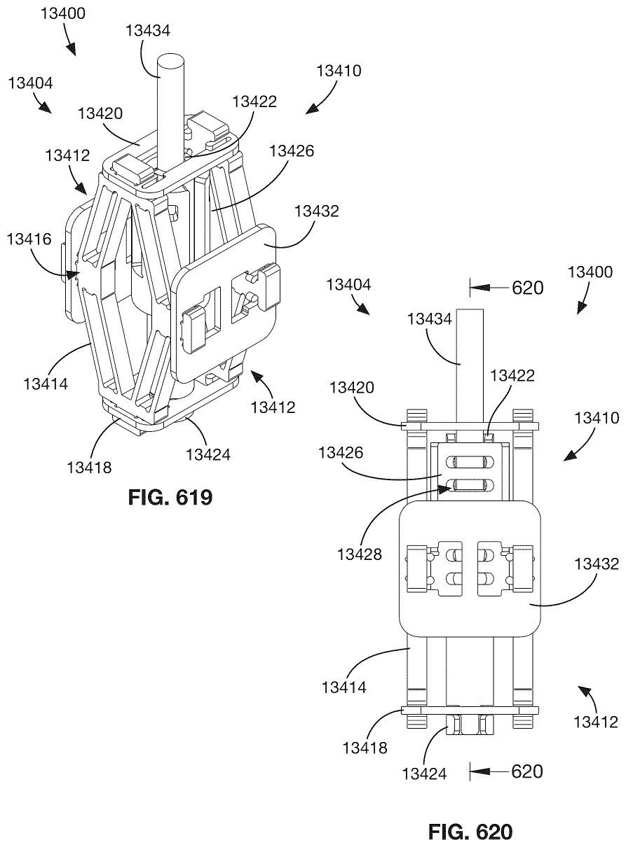

The latch member 13430 of the actuation tube 13422 can be moved between a latched and an unlatched condition. The actuation tube 13422 can include any number of latch members 13430 suitable for engaging corresponding openings 13428 in the latch tube 13426. Referring now to FIGS. 615-618 and 623, the latch member 13430 of the actuation tube 13422 is shown in an unlatched condition. When the latch member 13430 is in the unlatched condition, the actuation tube 13422 can be moved freely along the length of the latch tube 13426 to extend and retract the first end portion 13418, thereby expanding and contracting the expandable coaptation member or element 13410. In the latched condition, shown in FIGS. 619-622 and 624, the latch member 13430 in the latched condition engage the openings 13428 of the latch tube 13426 to secure the position of the actuation tube 13422 relative to the latch tube 13426.

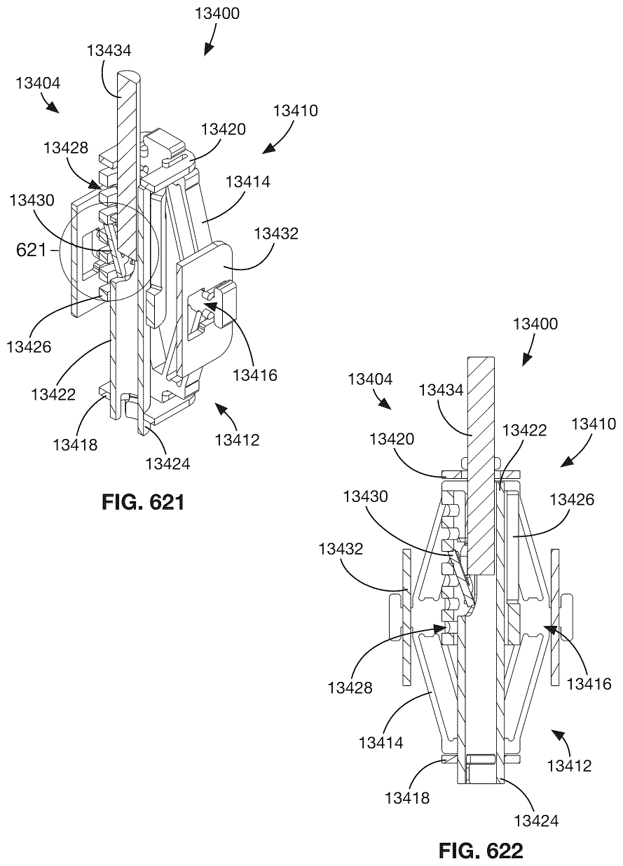

The latch member 13430 is integrally formed in the side of the actuation tube 13422 by laser-cutting a portion of the actuation tube 13422. The latch member 13430 is bent and shape-set in the latched condition so that in a free state the latch member 13430 is angled relative to a central axis of the actuation tube 13422. For example, as it shown in FIGS. 621-622, the proximal or upper end of the latch member 13430 is biased radially outward and the opposite lower or distal end of the latch member 13430 is biased radially inward. Alternatively, the proximal end of the latch member 13430 can be biased radially inward and the opposite distal end of the latch member 13430 can be biased radially outward. In some implementations, the expandable coaptation member or element 13410 is biased to expand or contract and the latch member 13430 is angled outward to oppose the bias of the expandable coaptation member or element 13410. For example, in an example where the expandable coaptation member or element 13410 is biased to cause the actuation tube 13422 to move in the proximal direction, the proximal end of the latch member 13430 can be biased radially outward so that the latch member 13430 engages the openings 13428 of the latch tube 13426 such that the proximal movement of the actuation tube 13422 is resisted by the latch member 13430.

The latch member 13430 is retained in the unlatched condition by the retaining or securing member 13434 that prohibits the latch member 13430 from pivoting into the latched condition. During implantation of the device 13400 the width of the expandable coaptation member or element 13410 is adjusted by extending and retracting the actuation tube 13422. When the desired width of the expandable coaptation member or element 13410 has been reached, the retention securing member 13434 is retracted from the actuation tube 13422 to allow the latch member 13430 to pivot into the latched condition to engage the openings 13428 of the latch tube 13426. The extension and retraction of the actuation tube 13422 causes the outer diameter of the expandable coaptation member or element 13410 to increase and decrease to accommodate different size and shape gaps left between the leaflets 20, 22 during diastole when the native heart valve closes around the device 13400. Locking the actuation tube 13422 in place relative to the latch tube 13426 via the latch member 13430 maintains the width of the expandable coaptation member or element 13410 against the force applied by the leaflets 20, 22. The retention or securing member 13434 can optionally have a tapered distal end to facilitate re-engagement of the latch member 13430 to cause the latch member 13430 to pivot from the latched condition to the unlatched condition, thereby enabling further adjustment of the width of the expandable coaptation member or element 13410.

The rigid portions 13414 of the struts 13412 and also an optional cover covering the struts 13412 can include shaped portions or can include flexible or pliable portions to allow the surface of the expandable coaptation member or element 13410 to conform to the shape of the native leaflets 20, 22 when the leaflets 20, 22 close against the implanted device 13400 to improve engagement between the struts 13412 and the native leaflets 20, 22. While four struts 13412 are shown, any number of struts 13412 can be combined to provided expanding and contracting coaptation surfaces. Different struts can include rigid portions 13414 having different shapes and lengths to provide differing rates of expansion and contraction and different maximum expansion positions to form a wide variety of differently shaped expandable coaptation members or elements 13410.

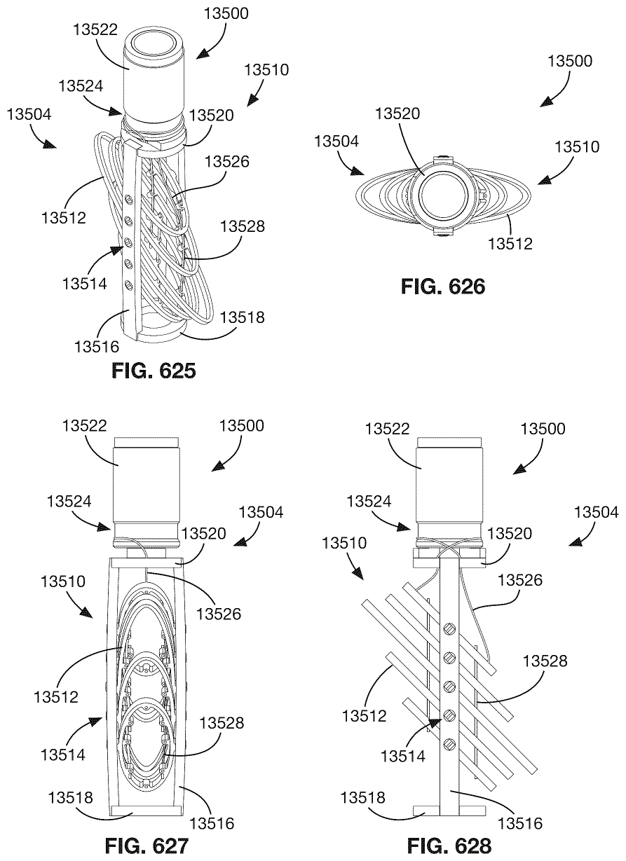

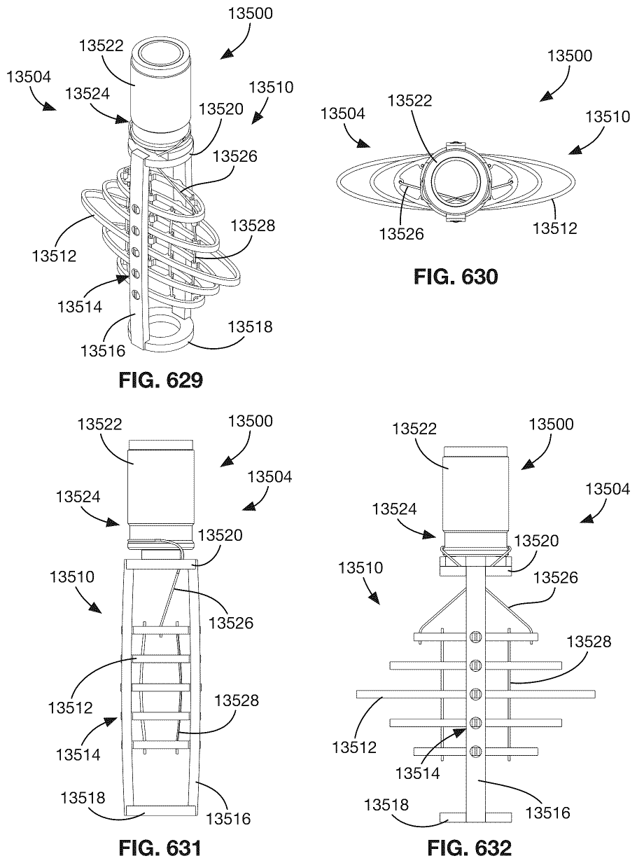

Referring now to FIGS. 625-632, an example expandable coaptation element 13510 (e.g., spacer, coaption element, gap filler, etc.) for use in a device 13500 (e.g., an implantable device, a spacer device, a valve repair device, etc.) is shown. The device 13500 can include any other features of devices discussed elsewhere in the present application, and the device 13500 can be positioned to engage valve tissue as part of any suitable valve repair system (e.g., any valve repair system disclosed in the present application). The coaptation device 13500 can be deployed from a delivery sheath or means for delivery by a pusher, such as a rod or tube as described above. The device 13500 can include a coaptation portion 13504 and an anchor portion having two or more anchors, such as the anchor portion and anchors described herein. The coaptation portion 13504 includes an expandable coaptation element 13510 that can be actuated between a contracted condition (FIGS. 625-628) and an expanded condition (FIGS. 629-632). An optional cover (not shown) can be included to cover a portion or all of the expandable coaptation member or element 13410.

The expandable coaptation member or element 13510 is formed from one or more rotatable frames 13512. The rotatable frames 13512 have a round shape, such as, for example, an oval shape, a circular shape, a lobed shape, or any other rounded shape suitable for filling the different size and shape gaps left between the leaflets 20, 22 during diastole when the native heart valve closes around the device 13500. The rotatable frames 13512 can each have a different size or shape, such as, for example, the frames in the middle of the expandable coaptation member or element 13510 can have a larger width as can be seen in FIGS. 625 and 629. The rotatable frames 13512 rotate around pivot axes 13514 that attach the rotatable frames 13512 to a longitudinal frame 13516 and allow the rotatable frames 13512 to rotate relative to the longitudinal frame 13516. The longitudinal frame 13516 is rigidly attached to and extends between a first or distal end portion 13518 to a second or proximal end portion 13520. One or more of the rotatable frames 13512 can include an attachment region (not shown) for attaching another component of the expandable coaptation member or element 13510 or of the coaptation device 13500.

The rotatable frames 13512 are moved between a collapsed condition and an expanded condition by the rotation of a rotatable actuation member 13522 (e.g., spool, etc.). The actuation spool 13522 includes a spool drum or arbor 13524 for engaging an actuation member 13526 (e.g., line, wire, etc.) that wraps around the spool arbor 13524 and attaches to one or more of the rotatable frames 13512. Rotating the actuation spool 13522 retracts a first length of the actuation member 13526 and allows a second length of the actuation member 13526 to extend such that the attached one or more rotatable frames 13512 rotate around their pivot axes 13514. One or more secondary actuation members 13528 can be used to connect the rotatable frames 13512 together so that all of the rotatable frames 13512 rotate simultaneously when the actuation spool 13522 is rotated. Rotating the actuation spool 13522 in the opposite direction retracts and releases the actuation members 13526 in an opposite fashion to move the rotatable frames 13512 from the expanded condition back to the collapsed condition. The rotation of the actuation spool 13522 causes the outer diameter of the expandable coaptation member or element 13510 to increase and decrease to accommodate different size and shape gaps left between the leaflets 20, 22 during diastole when the native heart valve closes around the device 13500.

The second end portion 13520 and/or the actuation spool 13522 can be attached to a proximal collar or head (not shown) of the device 13500 that can be coupled to a delivery mechanism for delivering the device 13500 within the native valve. The proximal collar or head can be engaged and actuated in a wide variety of ways described herein (such as, for example, via the mechanisms shown in FIGS. 417-482) to facilitate the implantation of the devices described herein, such as, for example, by opening and closing paddles of the device.

During implantation of the device 13500 the width of the expandable coaptation member or element 13510 is adjusted by rotating the actuation spool 13522 to cause the rotatable frames 13512 to rotate between the collapsed and expanded condition, thereby changing the lateral width and the shape of the expandable coaptation member or element 13510. When the desired width and shape of the expandable coaptation member or element 13510 has been reached, a retention member (e.g., lock, latch, etc., not shown) can be manipulated to lock the actuation spool 13522 to prohibit further rotation of the actuation spool 13522. Locking the actuation spool 13522 in place via the retention member maintains the width of the expandable coaptation member or element 13510 against the force applied by the leaflets 20, 22.

The rotatable frames 13512 and also an optional cover covering the rotatable frames 13512 can include shaped portions or can include flexible or pliable portions to allow the surface of the expandable coaptation member or element 13510 to conform to the shape of the native leaflets 20, 22 when the leaflets 20, 22 close against the implanted device 13500 to improve engagement between the rotatable frames 13512 and the native leaflets 20, 22. While five rotatable frames 13512 are shown, any number of rotatable frames 13512 can be combined to provided expanding and contracting coaptation surfaces. The rotatable frames 13512 can have different sizes and shapes to provide differing rates of expansion and contraction and different maximum expansion positions to form a wide variety of differently shaped expandable coaptation members or elements 13510.

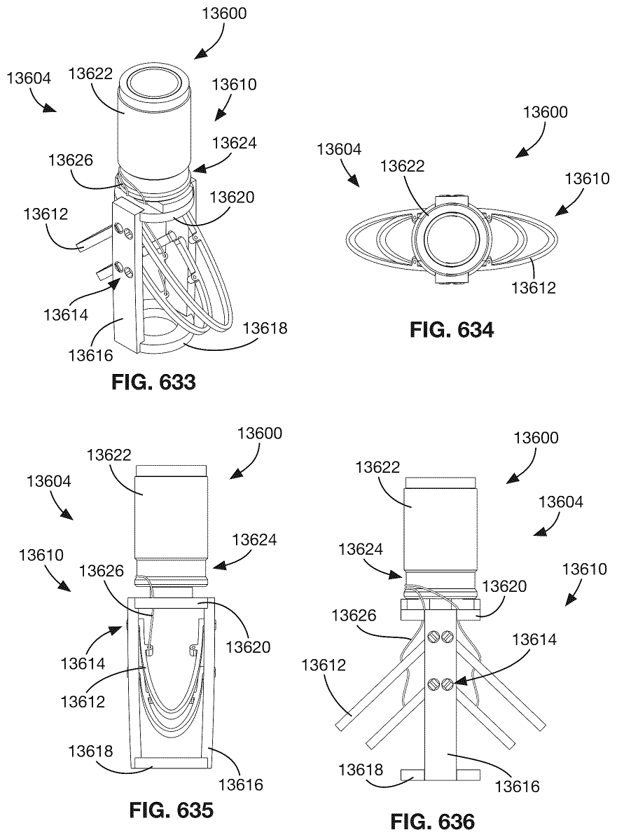

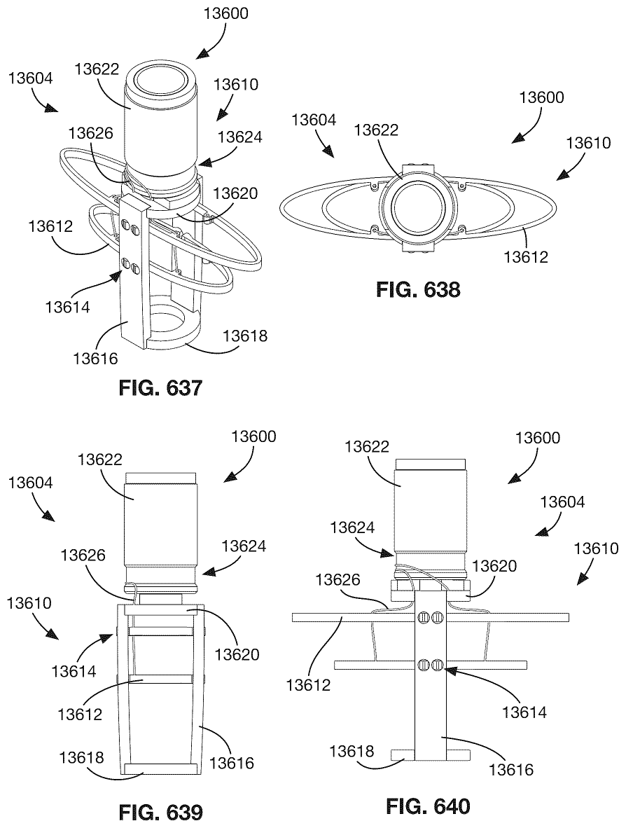

Referring now to FIGS. 633-640, an example expandable coaptation element 13610 (e.g., spacer, coaption element, gap filler, etc.) for use in a device 13600 (e.g., an implantable device, a spacer device, a valve repair device, etc.) is shown. The device 13600 can include any other features of devices discussed elsewhere in the present application, and the device 13600 can be positioned to engage valve tissue as part of any suitable valve repair system (e.g., any valve repair system disclosed in the present application). The coaptation device 13600 can be deployed from a delivery sheath or means for delivery by a pusher, such as a rod or tube as described above. The device 13600 can include a coaptation portion 13604 and an anchor portion having two or more anchors, such as the anchor portion and anchors described herein. The coaptation portion 13604 includes an expandable coaptation element 13610 that can be actuated between a contracted condition (FIGS. 633-636) and an expanded condition (FIGS. 637-640). An optional cover (not shown) can be included to cover a portion or all of the expandable coaptation member or element 13610.

The expandable coaptation member or element 13610 is formed from one or more rotatable frames 13612. The rotatable frames 13612 have a round shape, such as, for example, an oval shape, a circular shape, a lobed shape, or any other rounded shape suitable for filling the different size and shape gaps left between the leaflets 20, 22 during diastole when the native heart valve closes around the device 13600. The rotatable frames 13612 can each have a different size or shape, such as, for example, the frames near the proximal end of the expandable coaptation member or element 13610 can have a larger width as can be seen in FIGS. 633 and 637. The rotatable frames 13612 rotate around pivot axes 13614 that attach the rotatable frames 13612 to a longitudinal frame 13616 and allow the rotatable frames 13612 to rotate relative to the longitudinal frame 13616. The longitudinal frame 13616 is rigidly attached to and extends between a first or distal end portion 13618 to a second or proximal end portion 13620. One or more of the rotatable frames 13612 can include an attachment region (not shown) for attaching another component of the expandable coaptation member or element 13610 or of the coaptation device 13600.

The rotatable frames 13612 are moved between a collapsed condition and an expanded condition by the rotation of a rotatable actuation member 13622 (e.g., spool, etc.). The actuation spool 13622 includes a spool drum or arbor 13624 for engaging actuation members 13626 (e.g., line, suture, wire, etc.) that wrap around the spool arbor 13624 and attach to one or more of the rotatable frames 13612. Rotating the actuation spool 13622 in one direction retracts the actuation members 13626 to pivot the attached one or more rotatable frames 13612 around their pivot axes 13614 from a collapsed condition to the extended condition. Rotating the actuation spool 13622 in the opposite direction releases the actuation members 13626 to move the rotatable frames 13612 from the expanded condition back to the collapsed condition. The actuation members 13626 can be used to connect the rotatable frames 13612 together so that all of the rotatable frames 13612 rotate simultaneously when the actuation spool 13622 is rotated. The rotation of the actuation spool 13622 causes the outer diameter of the expandable coaptation member or element 13610 to increase and decrease to accommodate different size and shape gaps left between the leaflets 20, 22 during diastole when the native heart valve closes around the device 13600.

The second end portion 13620 and/or the actuation spool 13622 can be attached to a proximal collar or head (not shown) of the implantable spacer device 13600 that can be coupled to a delivery mechanism for delivering the device 13600 within the native valve. The proximal collar or head can be engaged and actuated in a wide variety of ways described herein (such as, for example, via the mechanisms shown in FIGS. 417-482) to facilitate the implantation of the devices described herein, such as, for example, by opening and closing paddles of the device.

During implantation of the device 13600 the width of the expandable coaptation member or element 13610 is adjusted by rotating the actuation spool 13622 to cause the rotatable frames 13612 to rotate between the collapsed and expanded condition, thereby changing the lateral width and the shape of the expandable coaptation member or element 13610. When the desired width and shape of the expandable coaptation member or element 13610 has been reached, a retention member (not shown) can be manipulated to lock the actuation spool 13622 to prohibit further rotation of the actuation spool 13622. Locking the actuation spool 13622 in place via the retention member maintains the width of the expandable coaptation member or element 13610 against the force applied by the leaflets 20, 22.

The rotatable frames 13612 and also an optional cover covering the rotatable frames 13612 can include shaped portions or can include flexible or pliable portions to allow the surface of the expandable coaptation member or element 13610 to conform to the shape of the native leaflets 20, 22 when the leaflets 20, 22 close against the implanted device 13600 to improve engagement between the rotatable frames 13612 and the native leaflets 20, 22. While four rotatable frames 13612 are shown, any number of rotatable frames 13612 can be combined to provided expanding and contracting coaptation surfaces. The rotatable frames 13612 can have different sizes and shapes to provide differing rates of expansion and contraction and different maximum expansion positions to form a wide variety of differently shaped expandable coaptation members or elements 13610.

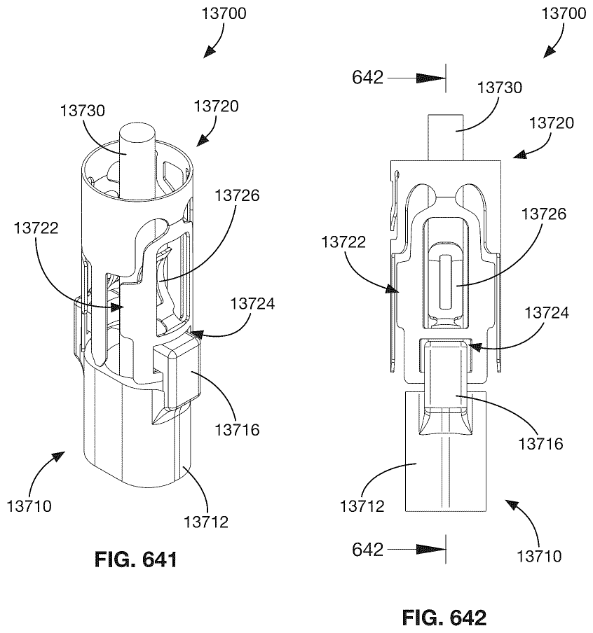

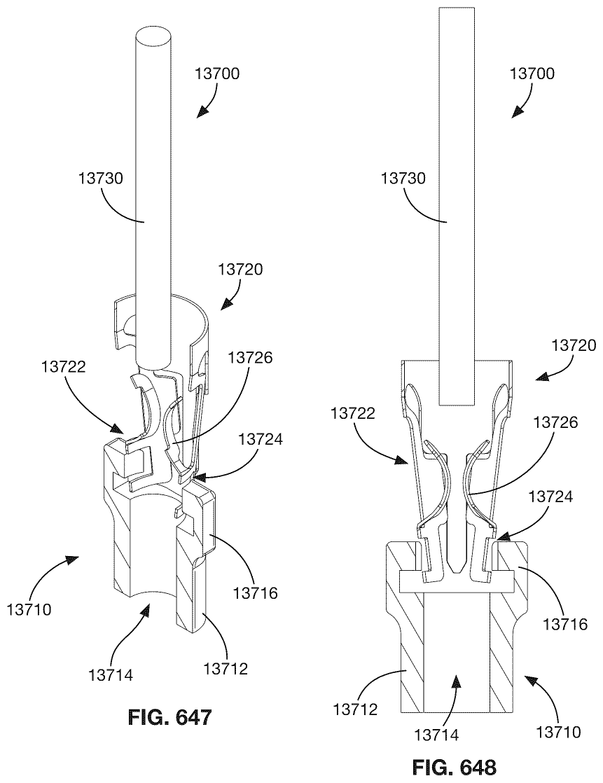

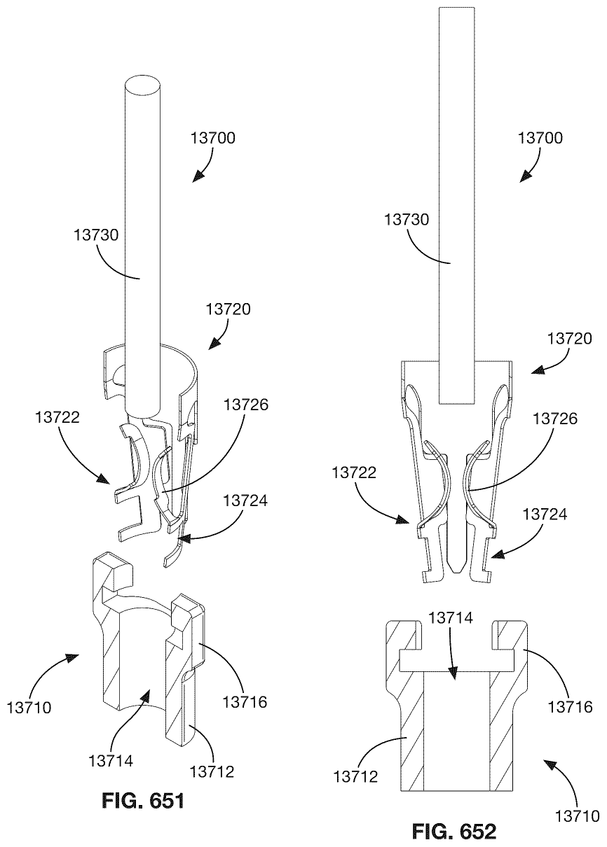

Referring now to FIGS. 641-652, an example coupler 13720 for use with a delivery system 13700 for an example device or implantable coaptation device 13710 is shown. The device 13710 can include any other features of devices discussed elsewhere in the present application, and the device 13710 can be positioned to engage valve tissue as part of any suitable valve repair system (e.g., any valve repair system disclosed in the present application). The coaptation device 13710 can be deployed from a delivery sheath or means for delivery by a pusher, such as a rod or tube as described above. The device 13710 can include a coaptation portion and an anchor portion having two more anchors, such as the coaptation portions, anchor portions and anchors described herein.

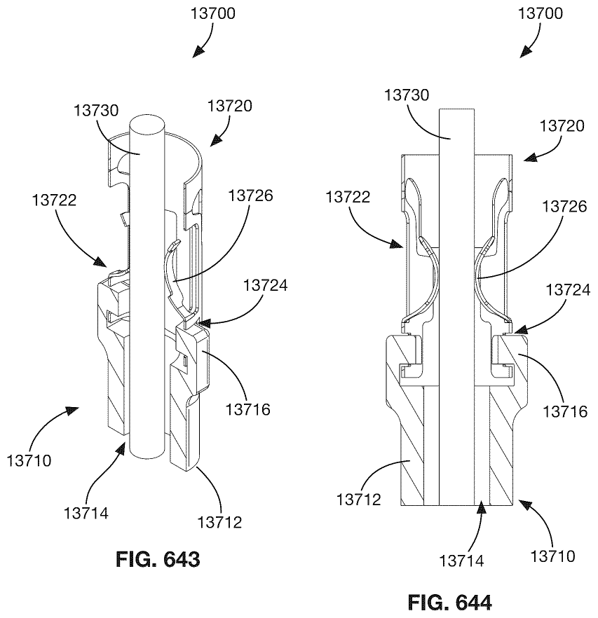

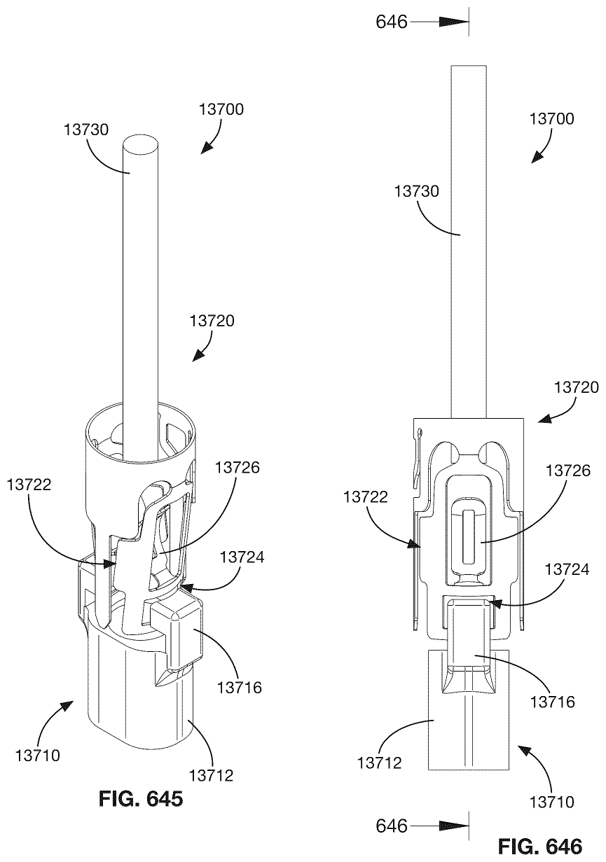

The device 13710 is similar to example devices described in the present application and includes a proximal collar 13712 having an axial opening 13714 (e.g., FIGS. 643-644) and radially disposed protrusions or fingers 13716. The protrusions or fingers 13716 extend proximally and then radially inward to provide an engagement location for the coupler 13720. The coupler 13720 has moveable arms or fingers 13722 that can be moved between open and closed positions. The moveable arms 13722 include radially disposed apertures or openings 13724 configured to engage the protrusions 13716 of the proximal collar 13712. The moveable arms 13722 can be moved radially inward (FIGS. 645-648) to disengage from the protrusions 13716. Inward movement of the moveable arms 13722 keeps the moveable arms 13722 clear from the native structures of the heart and other elements of the delivery system 13700 to reduce the chance that the moveable arms 13722 become entangled or catch during disengagement and retraction of the coupler 13720 from the device 13710 after implantation.

The moveable arms 13722 include engagement portions 13726 that protrude radially inward to engage an actuation element (e.g., actuation shaft, actuation rod, actuation tube, actuation wire, actuation line, etc.) and/or a width adjustment element. The actuation element and/or width adjustment element can take any of the forms disclosed herein and are illustrated as a shaft 13730 in the implementation illustrated by FIGS. 641-652. The engagement portions 13726 can be elastic so that engagement of the engagement portions 13726 with the shaft 13730 applies an outwardly directed bias force that maintains engagement between the openings 13724 and the protrusions 13716. That is, the presence of the shaft 13730 can cause the moveable arms 13722 to apply a positive force against the protrusions 13716 of the proximal collar 13712 even when the moveable arms 13722 are in a fully open position and are already in contact with the protrusions 13716. The engagement portions 13726 can have a rounded shape to more smoothly engage with the shaft 13730 as the shaft 13730 is inserted between the moveable arms 13722.

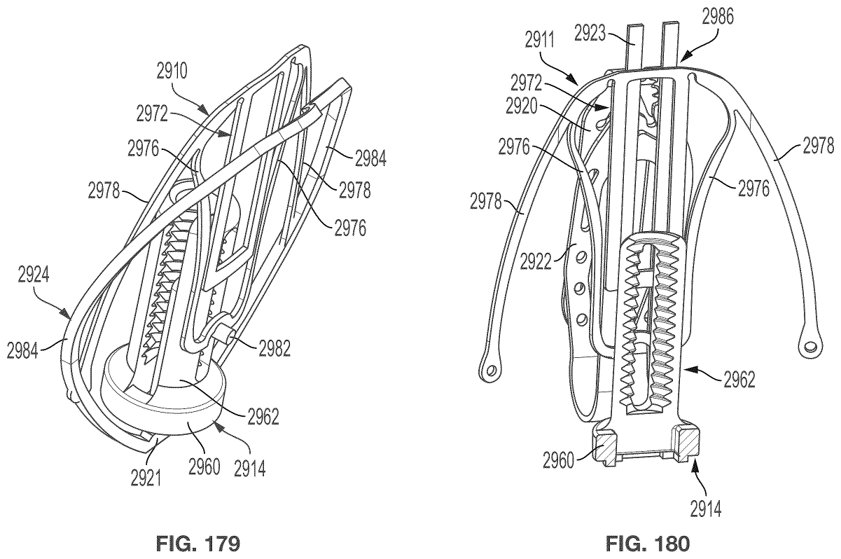

The shaft 13730 can extend through the coupler 13720 to the device 13710 to open and close the device 13710. The actuation shaft 13730 can be hollow so that a tethering line or suture (not shown) can be extended through the actuation shaft 13730 to the device 13710, such as, for example, the tether described above and shown in FIG. 179. While the actuation shaft 13730 remains between the moveable arms 13722 of the coupler 13720, the moveable arms 13722 are prohibited from moving inward and engagement between the coupler 13720 and the device 13710 is maintained.

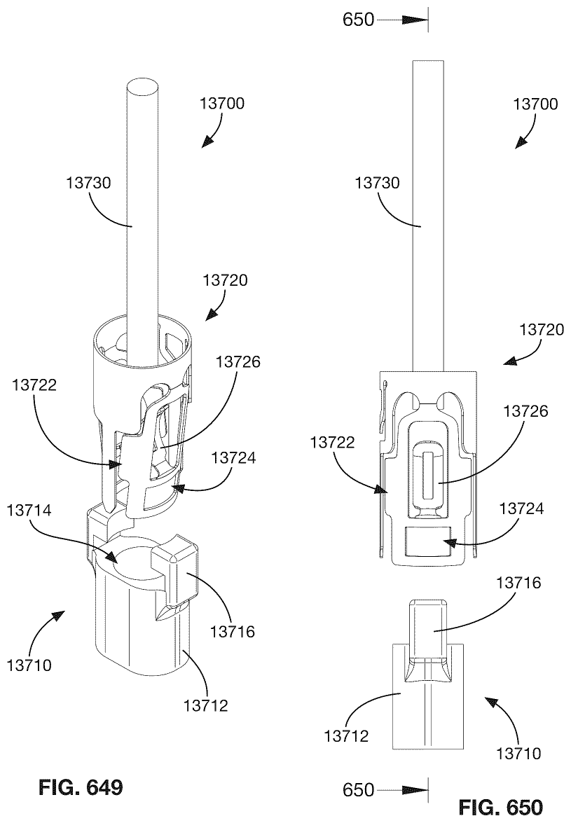

The moveable arms 13722 can be biased inward so that moving the actuation shaft 13730 in an extension or a distal direction through the coupler 13720 and between the engagement portions 13726 of the moveable arms 13722 spreads the moveable arms 13722 outwards so that the protrusions 13716 of the device 13710 engage the apertures 13724 of the moveable arms 13722. Moving the actuation shaft 13730 in a retraction or a proximal direction allows the moveable arms 13722 to move inward so that the apertures or openings 13724 disengage the protrusions 13716 of the device 13710, as can be seen in FIGS. 645-648. In this way the device 13710 can be released and/or recaptured by the coupler 13720. After the moveable arms 13722 have been allowed to disengage from the protrusions 13716 by way of the retraction of the shaft 13730, the coupler 13720 of the delivery system 13700 can also be moved in the retraction direction to disengage the coupler 13720 from the device 13710, as can be seen in FIGS. 649-652. The decoupling of the coupler 13720 from the device 13710 is performed in stages, that is, the actuation shaft 13730 is first moved in the retraction direction to allow the moveable arms 13722 to move inward to disengage from the protrusions 13716 of the device 13710 and then the entire coupler 13720 is retracted once the protrusions 13716 have been disengaged.

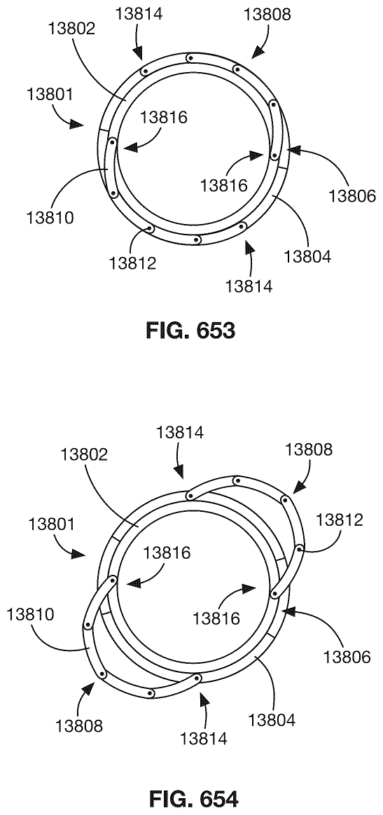

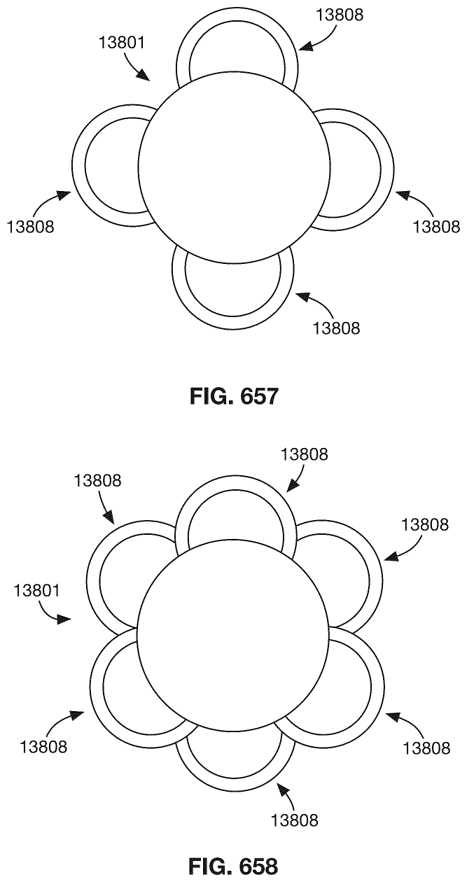

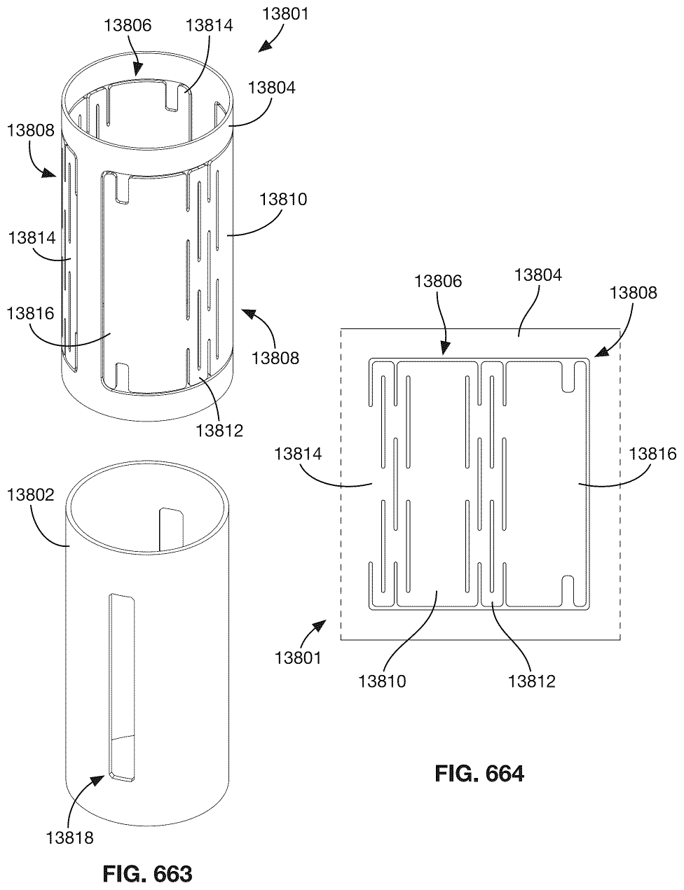

Referring now to FIGS. 653-664, an example expandable coaptation element 13808 (e.g., spacer, coaption element, gap filler, etc.) is shown. The expandable coaptation element forms part of a coaptation portion 13801 of an implantable coaptation device, such as any of the devices disclosed herein. Devices that include the coaptation portion 13801 can include any features of the devices discussed elsewhere in the present application, and the coaptation portion 13801 can be positioned to engage valve tissue as part of any suitable valve repair system (e.g., any valve repair system disclosed in the present application). Devices that include the coaptation portion 13801 can be deployed from a delivery sheath or means for delivery by a pusher, such as a rod or tube as described above. The devices can include a coaptation portion 13801 and an anchor portion having one, two or more anchors, such as any of the anchor portions and anchors described herein.

In some implementations, the coaptation portion 13801 includes a coaptation element 13808 (e.g., spacer, coaption element, gap filler, etc.) that can be actuated between a contracted condition (FIGS. 653, 659-660, and 663-664) and an expanded condition (FIGS. 654-658 and 661-662). An optional cover (not shown) can be included to cover a portion or all of the expandable coaptation member or element 13808.

In some implementations, the coaptation portion 13801 includes two concentrically arranged tubular structures: an inner tube 13802 and an outer tube or receiver 13804. In some implementations, the inner tube 13802 is sized to fit and rotate inside of the inner diameter of the outer tube or receiver 13804. In some implementations, the outer tube or receiver 13804 includes one or more openings 13806 that expose a portion of the inner tube 13802.

In some implementations, the expandable coaptation members or elements 13808 is formed from a plurality of links or flexible members 13810 jointed together by hinge portions 13812. In some implementations, the expandable coaptation members or elements 13808 extend from a first end 13814 hingeably attached to the outer tube or receiver 13804 to a second end 13816 that is hingeably attached to the inner tube 13802 through the opening 13806 in the outer tube or receiver 13804.

In some implementations, rotating the outer tube or receiver 13804 relative to the inner tube 13802 moves the first end 13814 of the flexible member rotationally closer to the second end 13816. This causes the plurality of links or flexible members 13810 to bulge or flex in an outward direction to cause the expandable coaptation members or elements 13808 to expand the size of the coaptation portion 13801. The rotation of the outer tube or receiver 13804 relative to the inner tube 13802 causes the outer aspect of the coaptation portion 13801 to increase and decrease to accommodate different size and shape gaps left between the leaflets 20, 22 during diastole when the native heart valve closes around the coaptation portion 13810.

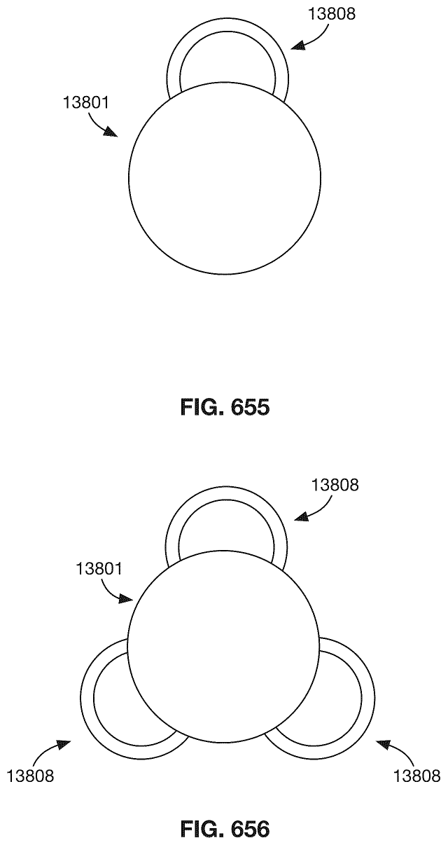

Referring now to FIGS. 653-658, various arrangements of the expandable coaptation members or elements 13808 of the coaptation portion 13801 are shown. The coaptation portion 13801 of the device 13800 can include one, two, or more expandable coaptation members or elements 13808 in various arrangements to fill different shape gaps formed when the leaflets 20, 22 close on the device 13800. Example arrangements of the expandable members or elements 13808 include the two oppositely arranged expandable coaptation members or elements 13808 shown in FIGS. 653-654 and the single expandable coaptation member or element 13808 shown in FIG. 655.

In some implementations, the expandable coaptation members or elements 13808 can be radially spaced apart around the coaptation portion 13801 as shown in FIGS. 656-658. The spacing of the expandable coaptation members or elements 13808 can be approximately equal or can be varied to form a variety of composite shapes for the coaptation portion 13801 when the expandable coaptation members or elements 13808 are in an expanded condition. The position of the expandable coaptation members or elements 13808 can also be varied longitudinally along the coaptation portion 13801, for example, to form two layers of expandable coaptation members or elements 13808 such as the six expandable coaptation members or elements 13808 shown in FIG. 658 that are arranged in two longitudinally spaced apart sets of three expandable coaptation members or elements 13808.

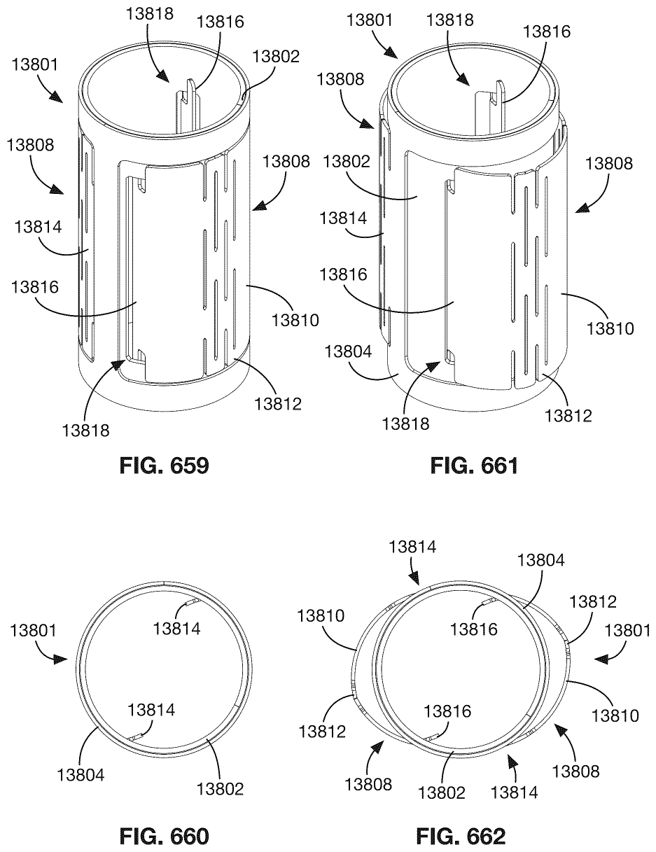

Referring now to FIGS. 659-664, an example implementation of a coaptation portion 13801 is shown. In the example shown in FIGS. 659-662, the expandable coaptation members or elements 13808 are formed from portions of the outer tube or receiver 13804. In some implementations, the first end 13814 of the expandable members or elements 13808 is joined to the outer tube or receiver 13804 by a hinge portion 13812 formed from a plurality of cuts in the outer tube or receiver 13804 that are mostly orthogonal to a bending direction of the links or flexible portions 13810 formed between hinge portions 13812. In some implementations, the second end 13816 of the expandable members or elements 13808 has a tab shape with ear-like projections. In some implementations, the second end 13816 is sized and shaped to fit within an opening 13818 in the inner tube 13802 so that the second end 13816 remains substantially stationary relative to the inner tube 13802 when the outer tube or receiver 13804 is rotated relative to the inner tube 13802 to expand the expandable members or elements 13808.

The first and second ends 13814, 13816 of the expandable members or elements 13808 can be attached to the outer and inner tubes 13804, 13802, respectively, in a wide variety of ways such as, for example, via a suture, a welded connection, a mechanical connection, a pinned connection, or the like.

Referring now to FIG. 664, a particular pattern of links or flexible portions 13810 and hinge portions 13812 cut from the outer tube or receiver 13804 is shown in FIG. 664. The cuts in the outer tube or receiver 13804 can be made in a wide variety of ways, such as, for example, via laser cutting, machining, wire electronic discharge machining, or the like. The expandable members or elements 13808 can have different sizes and shapes to provide differing rates of expansion and contraction and different maximum expansion positions to form a wide variety of differently shaped expandable coaptation portions 13801. To form these different sizes and shapes, the shape of the hinge portions 13812 and the links or flexible portions 13810 can take on a wide variety of forms to provide the expandable members or elements 13808 with a wide variety of shapes and bending qualities. For example, the links or flexible portions 13810 and hinge portions 13812 can have different widths and shapes so that the expandable members or elements 13808 have a variety of longitudinal cross-sectional shapes when in the expanded condition. Furthermore, the stiffness of the expandable members or elements 13808—and, consequently, the force required to expand the expandable members or elements 13808—can also be adjusted by changing the size and spacing of the cuts in the hinge portions 13812.

In some implementations, for example, links or flexible portions 13810 near the center of the expandable members or elements 13808 can be radially shorter and longitudinally shorter to provide a shape with a tighter radius when viewed from the end of the coaptation portion 13801 and a narrower outer most portion when viewed from the side of the coaptation portion 13801. The expandable members or elements 13808 and also an optional cover covering the expandable members or elements 13808 can include shaped portions or can include flexible or pliable portions to allow the surface of the expandable coaptation portion 13801 to conform to the shape of the native leaflets 20, 22 when the leaflets 20, 22 close against the implanted device 13800 to improve engagement between the expandable members or elements 13808 and the native leaflets 20, 22.

The inner and outer tubes 13802, 13804 can be formed from any material that is suitable for implantation in a patient and is sufficiently flexible for expansion. In some implementations, the outer tube or receiver 13804 can be formed from a metal such as steel or shape-memory alloy, such as Nitinol, to provide a bias in the expansion direction or contraction direction so that a rotational force is applied to or released from the inner tube 13802 or outer tube or receiver 13804 to cause the rotation of the outer tube or receiver 13804 relative to the inner tube 13802. That is, the expandable members or elements 13808 can be shape set in the expanded condition (e.g., FIG. 661) so that a rotational force is applied to the outer tube or receiver 13804 to retract the expandable members or elements 13808 and the rotational force is released to allow the expandable members or elements 13808 to expand. In some implementations, the expandable members or elements 13808 can be shape set in the contracted condition so that a rotational force is applied to the outer tube or receiver 13804 to expand the expandable members or elements 13808 and the rotational force is released to allow the expandable members or elements 13808 to retract.

In some implementations, during implantation of the device 13800 the width of the expandable coaptation members or elements 13808 is adjusted by rotating the outer tube or receiver 13804 relative to the inner tube 13802 to cause the expandable members or elements 13808 to flex or bend between the retracted and expanded condition, thereby changing the lateral width and shape of the coaptation portion 13801. When the desired width and shape of the expandable coaptation member or element 13808 has been reached, an optional retention member (not shown) can be manipulated to lock the outer tube or receiver 13804 relative to the inner tube 13802 to prohibit further rotation of the inner and outer tubes 13802, 13804. Locking the outer tube or receiver 13804 in place via a retention member maintains the width of the expandable coaptation member or element 13808 against the force applied by the leaflets 20, 22.

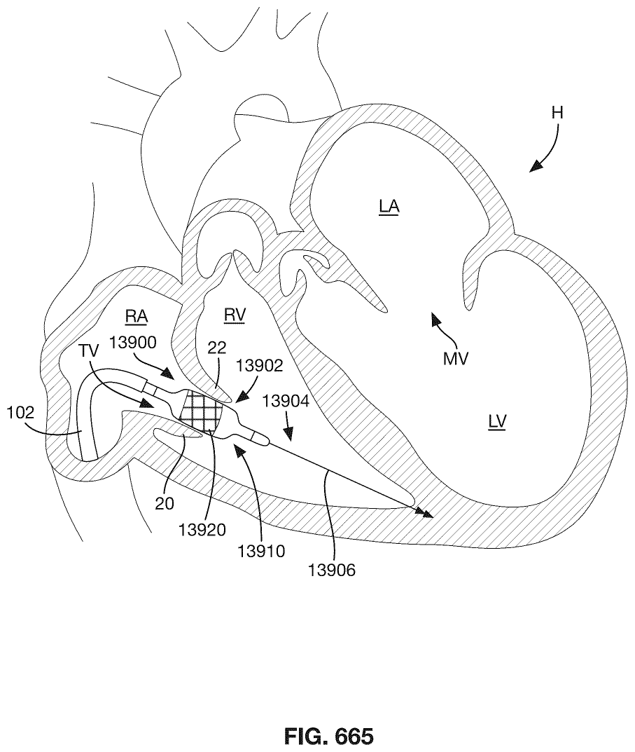

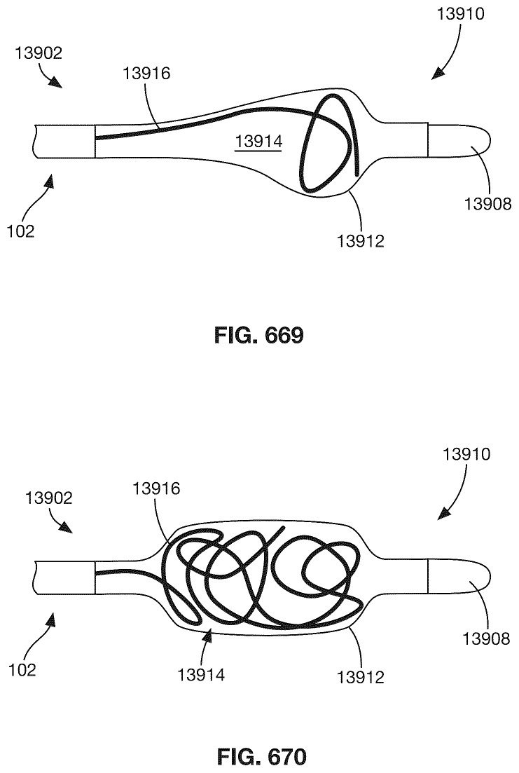

Referring now to FIGS. 665-672, an example expandable coaptation element 13910 (e.g., spacer, coaption element, gap filler, etc.) for use in a device 13900 (e.g., an implantable device, a spacer device, a valve repair device, a repair device, etc.) is shown. The device 13900 can include any other features of devices discussed elsewhere in the present application, and the device 13900 can be positioned to engage valve tissue as part of any suitable valve repair system (e.g., any valve repair system disclosed in the present application).

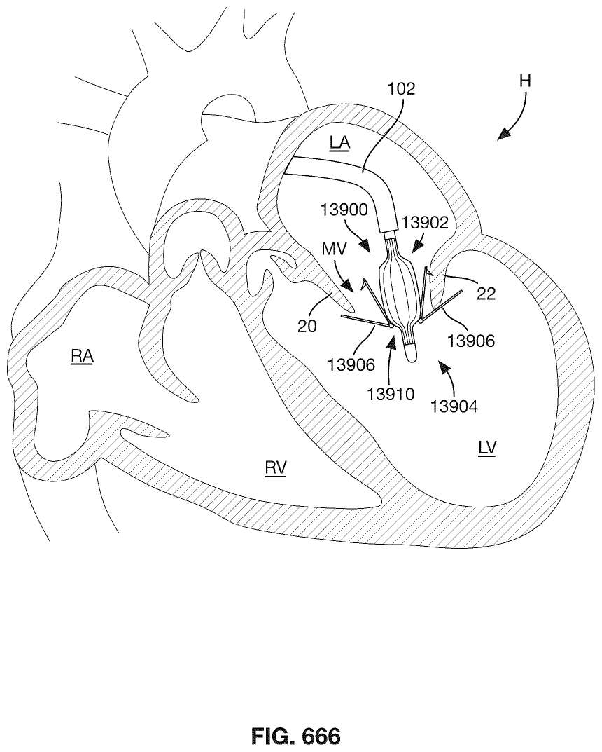

In some implementations, the coaptation device 13900 can be deployed from a delivery sheath or means for delivery 102 by a pusher, such as a rod or tube as described above. The device 13900 can be implanted in the tricuspid valve TV (FIG. 665) or the mitral valve MV (FIG. 666).

In some implementations, the device 13900 can include a coaptation portion 13902 and an anchor portion 13904 having two or more anchors 13906, such as the anchor portion and anchors described herein. Providing the device 13900 with an expandable coaptation element 13910 allows the device 13900 to be useful in heart valve openings in a wide range of sizes.

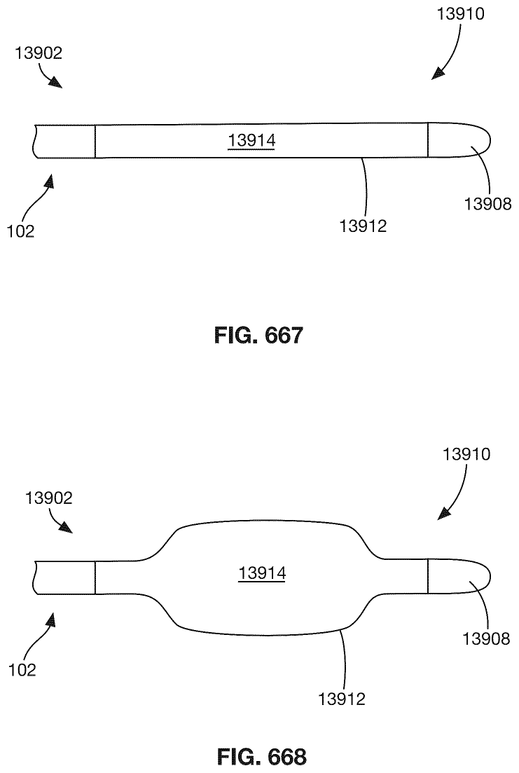

In some implementations, the coaptation portion 13902 includes a coaptation element 13910 (e.g., spacer, coaption element, gap filler, bag, pouch, etc.) that can be actuated between a contracted condition (FIG. 667) and an expanded condition (FIGS. 665-666 and 668-672). An optional cover (not shown) can be included to cover a portion or all of the expandable coaptation member or element 13910. In some implementations, the anchor portion 13904 can include a wide variety of anchors 13906, such as, for example, clasp-like anchors (see, e.g., FIG. 666) can be opened and closed to grasp the valve leaflets 20, 22 or a tether-like anchor (see, e.g., FIG. 665) can be used that is secured by barbs or hooks into one or more locations in the wall of the heart and includes an elongated member extending from the barbed or hooked portion to the device 13900. In some implementations an anchor portion 13906 extends from the coaptation portion 13902 and anchors in the right atrium RA, in addition to the anchor portion 13906 that anchors in the right ventricle.

Referring now to FIGS. 667-672, an expandable coaptation element 13910 for use in a device 13900 being expanded from an unexpanded condition (FIG. 667) to an expanded condition (FIGS. 668-672) is shown. In some implementations, the expandable coaptation element 13910 includes a flexible enclosure 13912 that forms an expandable cavity 13914. The flexible enclosure 13912 can optionally include a closed end 13908.

In some implementations, the flexible enclosure 13912 can be expanded from the unexpanded condition (FIG. 667) to the expanded condition (FIG. 668) by introducing a filling material in the expandable cavity 13914, such as, for example, a saline solution and/or by including or being formed from self-expanding elements, such as, for example, a frame (see FIG. 666) formed from a shape memory alloy such as Nitinol. The expandable cavity 13914 can also be filled with a wide variety of filling materials to provide additional stiffness to the expandable coaptation element 13910. Filling the expandable cavity 13914 with filling material can also cause or contribute to the expansion of the flexible enclosure 13912. In some implementations, the expandable coaptation element 13910 expands to a diameter in a range of about 10 millimeters to about 20 millimeters.

The flexible enclosure 13912 can be formed from a wide variety of materials, such as, for example, a woven textile material, an extruded polymer material, a blow-molded polymer material, or the like. When formed from or coated with a moldable material, the flexible enclosure 13912 can be molded in the expanded condition to form a predetermined shape when filled with a filling material to cause the flexible enclosure 13912 to expand. The material used to form the flexible enclosure 13912 can optionally be permeable to allow blood to pass through the expandable coaptation element 13910 or to enter the expandable cavity 13914 to interact with the filling material. In some implementations, the flexible enclosure 13912 is formed from a polymer material and is coated with pharmaceuticals to encourage or discourage tissue growth. Polymers used to form the flexible enclosure 13912 can be resorbable polymers.

The surface of the flexible enclosure 13912 can be formed with a wide variety of surface finishes, such as, for example, a smooth surface, a rough surface, a coated surface, a treated surface, or the like. In some implementations of the flexible enclosure 13912 formed from a woven textile material, the surface of the flexible enclosure 13912 can be made smooth via the use of very fine yarns and be made bulky with the use of textured or high surface area yarns. In some implementations, the flexible enclosure 13912 is coated with a polymer film to, for example, prevent or inhibit thrombus adhesion to the coaptation element 13910. The surface of the flexible enclosure 13912 in some implementations can be plasma treated.

Optionally, the flexible enclosure 13912 can include a contrasting surface 13920 (FIG. 665) formed in a different fashion than the remainder of the flexible enclosure 13912. For example, the contrasting surface 13920 can have a finish that is more rough or smoother than the surface of the flexible enclosure 13912. In some implementations, the contrasting surface 13920 can be treated with a substance to encourage or discourage tissue ingrowth in a particular portion of the expandable coaptation element 13910. In this way, for example, the leaflets 20, 22 could be discouraged from attaching to a contrasting surface 13920 arranged where the leaflets contact the expandable coaptation element 13910 during diastole when the native heart valve closes around the device 13900. In some implementations, the flexible enclosure 13912 can be permeable and include a contrasting surface 13920 that is treated to promote clotting in a desired location to gradually form a blood clot to block blood flow through the device 13910 partially or fully.

Referring now to FIGS. 668-672, the expandable coaptation element 13910 can be filled with an elongated filling element 13916 (e.g., coil, wire, yarn, filling, etc.) that is inserted through the delivery sheath or means for delivery 102 and into the expandable cavity 13914 formed by the flexible enclosure 13912. In some implementations, as shown in FIG. 668, the expandable cavity 13914 can be inflated with saline (or another suitable fluid) prior to the insertion of the elongated filling element 13916.

In some implementations, as shown in FIG. 369, the expandable cavity 13914 is expanded by virtue of the introduction of the elongated filling element 13916. In some implementations, the elongated filling element 13916 can be formed from a coiled or braided material, such as a textile coil, a metal coil, a polymer coil, or any suitable combination thereof. In some implementations, the elongated filling element 13916 is flexible such that upon engaging the flexible enclosure 13912, the elongated filling element 13916 bends and turns repeatedly as the expandable cavity 13914 is filled with the elongated filling element 13916. In some implementations, the elongated filling element 13916 can also include radiopaque markers or be made from a radiopaque material so that the elongated filling element 13916 can be detected by the physician during a procedure. In some implementations, the elongated filling element 13916 is a metal coil that is coiled with fibrous materials such as PET or PGA/PLGA yard to provide additional surface area to the elongated filling element 13916 to facilitate, for example, a more aggressive clotting response.

Referring now to FIG. 670, the expandable cavity 13914 is filled with the elongated filling element 13916 to provide resistance to compression of the expandable coaptation element 13910 as the leaflets 20, 22 close against the flexible enclosure 13912. In some implementations, the flexible enclosure 13912 is formed from sufficiently thick and/or rigid material such that the shapes of the various turns and bends of the elongated filling element 13916 are not visible on the exterior surface of the flexible enclosure 13912. In some implementations, the expandable cavity 13914 is filled with the elongated filling element 13916 until a desired size and/or relative stiffness of the expandable coaptation element 13910 is met. In some implementations, the stiffness of the expandable coaptation element 13910 depends on the quantity and material of the elongated filling element 13916. That is, the greater the quantity and the greater the stiffness of the material of the elongated filling element 13916, the greater the stiffness of the expandable coaptation element 13910.

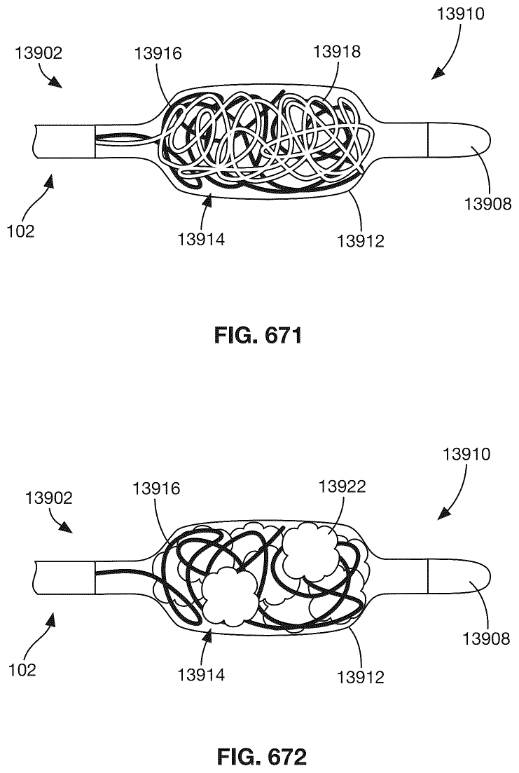

Referring now to FIGS. 671-672, the expandable coaptation element 13910 is shown filled with the elongated filling element 13916 and optional auxiliary or secondary filling materials. As can be seen in FIG. 671, in some implementations a second elongated filling element 13918 is inserted into the expandable cavity 13914 simultaneously with or subsequent to delivery of the elongated filling element 13916. The second elongated filling element 13918 can vary in stiffness, diameter, shape, and/or material from the elongated filling element 13916. In some implementations, the elongated filling element 13916 provides stiffness and structure to the expandable coaptation element 13910 and the second elongated filling element 13918 can be formed from a more flexible or pliable material to fill gaps in the elongated filling element 13916 and/or to provide additional functionality to the expandable coaptation element 13910. For example, the second elongated filling element 13918 can include a fibrous material or be treated or coated with a material to encourage clot formation. In some implementations, one or both of elongated filling element 13916 and the second elongated filling element 13918 are coated with a hydrogel that expands after delivery of the elongated filling elements 13916, 13918.

Referring now to FIG. 672, an example is shown that includes a plurality of pieces of filling material 13922 that are inserted into the expandable cavity 13914 simultaneously with or subsequent to delivery of the elongated filling element 13916. In some implementations, the pieces of filling 13922 are attached to the elongated filling element 13916. The pieces of filling material 13922 can take on a wide variety of shapes, sizes, stiffness, and the like and can be consistent or can vary from one another. In some implementations, the pieces of filling material 13922 are formed from a material that is more flexible or pliable than that of the elongated filling element 13916 to fill gaps in the elongated filling element 13916 and/or to provide additional functionality to the expandable coaptation element 13910. For example, the pieces of filling material 13922 can include a fibrous material or be treated or coated with a material to encourage clot formation. In some implementations, one or both of elongated filling element 13916 and the pieces of filling material 13922 are coated with a hydrogel that expands after delivery of the elongated filling element 13916 and/or the pieces of filling material 13922.

In some implementations, during implantation of the device 13900 the size, such as the width, of the expandable coaptation members or elements 13910 is expanded to a desired size by filling the expandable cavity with the elongated filling member 13916 (e.g., wire, coil, strand, yarn, tufts, fibers, etc.) optionally preceded by filling with saline or another suitable liquid. The optional second elongated filling element 13918 and/or the optional fibrous elements 13922 can also be introduced into the expandable cavity 13914. When the desired width and shape of the expandable coaptation member or element 13910 has been reached, the elongated filling member 13916 can be broken off or otherwise cut and secured within the expandable cavity 13914 so that the size and firmness of the expandable coaptation element 13910 is maintained against the force applied by the leaflets 20, 22.

### EXAMPLES

Example 1. A heart valve repair system comprising: (A) a device comprising a proximal head and an adjustable member attached to the proximal head; and (B) a delivery system comprising: (i) a distal gripper for deploying the device in a patient; (ii) a coupling assembly for releasably coupling the distal gripper to the proximal head, wherein the coupling assembly transmits rotational movement of the distal gripper to the adjustable member via the proximal head when the coupling assembly is in a coupled condition; and (iii) a retention member that extends or is extendable through the delivery system and into the coupling assembly for securing the coupling assembly in the coupled condition.

Example 2. The heart valve repair system of example 1, wherein the retention member extends through the coupling assembly and/or engages the adjustable member.

Example 3. The heart valve repair system of any of the above examples, wherein the proximal head is releasably attached to the adjustable member.

Example 4. The heart valve repair system of any of the above examples, wherein: (i) the proximal head comprises a plurality of engagement protrusions; and (ii) the distal gripper comprises a plurality of engagement protrusions for intermeshing with the engagement protrusions of the proximal head.

Example 5. The heart valve repair system of any of the above examples, wherein: (i) the retention member comprises a threaded distal end; and (ii) the proximal head comprises a threaded opening for receiving the threaded distal end; wherein threading the threaded distal end of the retention member into the threaded opening of the proximal head retains the coupling assembly in the coupled condition.

Example 6. The heart valve repair system of any of the above examples, wherein: (i) the distal gripper comprises a plurality of flexible fingers, each flexible finger comprising an engaging portion; and (ii) the proximal head comprises an opening for receiving the flexible fingers and windows for receiving the engaging portions of the flexible fingers.

Example 7. The heart valve repair system of example 6, wherein extending the retention member between the flexible fingers prohibits the engaging portions of the flexible fingers from disengaging from the windows.

Example 8. The heart valve repair system of example 7, wherein the flexible fingers are biased inward so that removal of the retention member allows the flexible fingers to move inward so that the engaging portions disengage from the windows.

Example 9. The device of any of the above examples, wherein: (i) the distal gripper comprises a pivoting engaging member; and (ii) the proximal head comprises a window for receiving the pivoting engaging member.

Example 10. The heart valve repair system of example 9, wherein extending the retention member into the distal gripper engages the pivoting engaging member to cause the pivoting engaging member to pivot outwards to engage the window of the proximal head.

Example 11. The heart valve repair system of example 10, wherein the pivoting engaging member is biased to pivot in an inward direction to facilitate disengagement from the window of the proximal head.

Example 12. The heart valve repair system of any of the above examples, wherein: (i) the distal gripper comprises a sliding engaging member; and (ii) the proximal head comprises a window for receiving the sliding engaging member.

Example 13. The heart valve repair system of example 12, wherein extending the retention member into the distal gripper engages the sliding engaging member to cause the sliding engaging member to slide outwards to engage the window of the proximal head.

Example 14. The heart valve repair system of example 13, wherein the sliding engaging member is biased to slide in an inward direction to facilitate disengagement from the window of the proximal head.

Example 15. The heart valve repair system of any of the above examples, wherein: (i) the distal gripper comprises a deployable engaging member attached to the distal gripper via a linkage; and (ii) the proximal head comprises a recess for receiving the deployable engaging member.

Example 16. The heart valve repair system of example 15, wherein distal movement of the distal gripper causes the deployable engaging member to move into the recess of the proximal head.

Example 17. The heart valve repair system of example 16, wherein extending the retention member between the distal gripper and the deployable engaging member secures the deployable engaging member in the recess of the proximal head.

Example 18. The heart valve repair system of any of the above examples, wherein: (i) the distal gripper comprises a slot; and (ii) the proximal head comprises a laterally extending pin for engaging the slot of the distal gripper.

Example 19. The heart valve repair system of example 18, wherein extending the retention member within the distal gripper secures the laterally extending pin within the slot of the distal gripper.

Example 20. The heart valve repair system of any of the above examples, wherein: (i) the distal gripper comprises a keyed end having an engaging member; and (ii) the proximal head comprises an opening configured to receive the keyed end of the distal gripper only when the distal gripper is offset axially from the proximal head.

Example 21. The heart valve repair system of example 20, wherein extending the retention member through the distal gripper and the proximal head secures the engaging member of the keyed end in a window of the proximal head.

Example 22. The heart valve repair system of any of the above examples, wherein: (i) the distal gripper comprises a window; and (ii) the proximal head comprises an engaging protrusion configured to extend into and be received by the window of the distal gripper.

Example 23. The heart valve repair system of example 22, wherein extending the retention member through the distal gripper and the proximal head secures the engaging member of the proximal head in the window of the distal gripper.

Example 24. The heart valve repair system of any of the above examples, wherein: (i) the distal gripper comprises a plurality of flexible fingers, each flexible finger comprising a window; and (ii) the proximal head comprises an opening for receiving the flexible fingers and engaging portions for engaging the windows of the flexible fingers.

Example 25. The heart valve repair system of example 24, wherein extending the retention member between the flexible fingers prohibits the windows of the flexible fingers from disengaging from the engaging portions.

Example 26. The heart valve repair system of example 25, wherein the flexible fingers are biased inward so that removal of the retention member allows the flexible fingers to move inward so that the windows disengage from the engaging portions.

Example 27. The heart valve repair system of any of the above examples, wherein: (i) the distal gripper comprises a deployable engaging member that is attached to the distal gripper by a linkage; and (ii) the proximal head comprises an opening for receiving the distal gripper and a recess within the opening for receiving the deployable engaging member.

Example 28. The heart valve repair system of example 27, wherein when the deployable engaging member contacts a bottom of the opening in the proximal head, the deployable engaging member moves radially outward and into the recess.

Example 29. The heart valve repair system of any of examples 27-28, wherein the retention member extends between the deployable engaging member and the distal gripper to prohibit the distal gripper from moving from a deployed condition to a retracted condition.

Example 30. The heart valve repair system of any of the above examples, wherein: (i) the proximal head comprises a laterally oriented pin; (ii) the distal gripper comprises a slot for engaging the laterally oriented pin and an axial opening for receiving the retention member; and (iii) the retention member extends beyond the laterally oriented pin of the proximal head to retain the laterally oriented pin in the slot of the distal gripper.

Example 31. The heart valve repair system of example 30, wherein the distal gripper comprises a tapered opening for guiding the retention member toward an open end of the slot.

Example 32. The heart valve repair system of example 31, wherein the distal gripper and the proximal head are axially aligned when the laterally oriented pin is retained in the slot by the retention member.

Example 33. An actuation mechanism for a device, the actuation mechanism comprising: (i) an outer tube comprising a plurality of openings; (ii) a latch tube attached to an adjustable member, the latch tube comprising a latch member and a threaded opening; and (iii) an actuation member having a threaded portion that can be threadably attached to the threaded opening latch tube and a tapered distal end; wherein when the threaded portion of the actuation member is threaded into the threaded opening of the latch tube, the tapered distal end engages the latch member and retains the latch member in an unlatched condition so that the latch tube can be moved to a desired position in the outer tube; and wherein the actuation member can be unthreaded from the threaded opening of the latch tube to disengage the tapered distal end from the latch member to facilitate movement of the latch member to the latched condition wherein the latch member engages one of the plurality of openings of the outer tube.

Example 34. The actuation mechanism of example 33, wherein the latch member is laser cut from the latch tube.

Example 35. The actuation mechanism of any of examples 33-34, wherein the latch member is biased toward the latched condition.

Example 36. The actuation mechanism of any of examples 33-35, wherein the latch tube further comprises an orientation tab that extends through an orientation slot of the outer tube to prohibit relative rotation of the latch tube and outer tube.

Example 37. An actuation mechanism for a device, the actuation mechanism comprising: (i) an outer tube comprising a plurality of openings; (ii) a latch tube attached to an adjustable member, the latch tube comprising a latch member and a proximal end that forms one half of a coupling connection; (iii) an actuation tube having a distal end that forms the other half of the coupling connection and is configured to releasably couple to the proximal end of the latch tube; and (iv) a securing member that extends through the actuation tube and latch tube to prohibit the decoupling of the coupling connection when the actuation tube and latch tube are in a coupled condition, the securing member having a tapered distal end; wherein when the securing member is inserted into the latch tube, the tapered distal end engages the latch member and retains the latch member in an unlatched condition so that the latch tube can be moved to a desired position in the outer tube; wherein the securing member can be withdrawn from the latch tube to disengage the tapered distal end from the latch member to facilitate movement of the latch member to the latched condition wherein the latch member engages one of the plurality of openings of the outer tube; and wherein the securing member can be withdrawn from the coupling connection to allow the actuation tube to disengage from the latch tube.

Example 38. The actuation mechanism of example 37, wherein the latch member is laser cut from the latch tube.

Example 39. The actuation mechanism of any of examples 37-38, wherein the latch member is biased toward the latched condition.

Example 40. The actuation mechanism of any of examples 37-39, wherein the latch tube further comprises an orientation pin that extends through an orientation slot of the outer tube to prohibit relative rotation of the latch tube and outer tube.

Example 41. The actuation mechanism of any of examples 37-40, wherein withdrawing the securing member from the coupling connection allows the actuation tube to move laterally relative to the latch tube to disengage the coupling connection.

Example 42. An actuation mechanism for a device, the actuation mechanism comprising: (i) an actuation member releasably attached to an adjustable member, the adjustable member comprising a plurality of notches; (ii) a latch tube comprising a plurality of latch members spaced apart longitudinally along the latch tube and biased to move inward toward a latched condition; and (iii) a release tube arranged between the adjustable member and the latch tube, wherein the release tube prohibits the latch members from moving from an unlatched condition to the latched condition to permit the adjustable member can be moved to a desired position; wherein withdrawing the release tube distally permits at least one of the latch members to move inward from the unlatched condition to the latched condition by engaging one of the notches of the adjustable member.

Example 43. The actuation mechanism of example 42, wherein the latch members are laser cut from the latch tube.

Example 44. The actuation mechanism of any of examples 42-43, wherein the actuation member is a suture.

Example 45. A handle release assembly of a delivery system for a device, the handle release assembly comprising: (i) an actuation element extending from a distal end for engaging the device to a proximal end that is secured to an actuation element adaptor; (ii) a width adjustment element extending within the actuation element from a distal end for engaging the device to a proximal end that is secured to a width adjustment element adaptor; (iii) a connector body having a central lumen through which an actuation element and width adjustment element extend, the connector body comprising a coupling portion that includes a retention groove; (iv) an inner body comprising attachment members for engaging the coupling portion and the retention groove of the connector body, a recess for receiving the actuation element adaptor, a catch, and side slots; (v) an outer body comprising a proximal stop, a latch arm having a protrusion for engaging the catch of the inner body to prohibit relative movement of the outer body and the inner body, and side slots, wherein the width adjustment element adaptor extends through the side slots of the inner body and the side slots of the outer body; and (vi) a gripping portion slidably attached to the outer body; wherein sliding the gripping portion in the proximal direction exposes the latch arm of the outer body to permit the latch arm to disengage from the catch of the inner body; wherein after disengaging the latch arm, the outer body is moved proximally by application of further proximal force on the gripping portion to move the outer body proximally so that the side slots engage the width adjustment element adaptor to move the width adjustment element in the proximal direction; and wherein further proximal movement of the gripping portion and the outer body exposes the attachment members of the inner body and causes the width adjustment element adaptor to engage the side slots of the inner body to disengage the attachment members from the retention groove of the coupling portion of the connector body and to pull the actuation element in the proximal direction.

Example 46 A handle release assembly of a delivery system for a device, the handle release assembly comprising: (i) an actuation element extending from a distal end for engaging the device to a proximal end that is secured to an actuation element adaptor; (ii) a width adjustment element extending within the actuation element from a distal end for engaging the device to a proximal end that is secured to a width adjustment element adaptor; (iii) a connector body having a central lumen through which an actuation element and width adjustment element extend, the connector body comprising a coupling portion that includes a retention groove; (iv) a moveable body comprising attachment members for engaging the coupling portion and the retention groove of the connector body, a recess for receiving the actuation element adaptor, and side slots, wherein the width adjustment element adaptor extends through the side slots of the moveable body; and (v) a gripping portion slidably attached to the moveable body; wherein sliding the gripping portion in the proximal direction engages the width adjustment element adaptor to move the width adjustment element in the proximal direction; and wherein further proximal movement of the gripping portion and the outer body exposes the attachment members of the moveable body and causes the width adjustment element adaptor to engage the side slots of the inner body to disengage the attachment members from the retention groove of the coupling portion of the connector body and to pull the actuation element in the proximal direction.

Example 47. A clasp for a device, the clasp comprising: (i) a fixed arm having a curved shape; (ii) a moveable arm having a curved shape that is complementary to the curved shape of the fixed arm; and (iii) a joint portion that hingeably connects the moveable arm to the fixed arm.

Example 48. The clasp of example 47, further comprising a gripping protrusion extending from at least one of the fixed arm and the moveable arm.

Example 49. The clasp of any of examples 47-48, wherein the gripping protrusion has a rounded end.

Example 50. The clasp of any of examples 47-49, wherein the curved shape is a sinusoidal shape.

Example 51. The clasp of any of examples 47-49, wherein the curved shape is a triangular shape.

Example 52. The clasp of any of examples 47-49, wherein the curved shape is a sawtooth shape.

Example 53. The clasp of any of examples 47-49, wherein the curved shape has a peak arranged proximal to the joint portion and a trough arranged distal of the peak.

Example 54. The clasp of any of examples 47-49, wherein the curved shape has a trough arranged proximal to the joint portion and a peak arranged distal of the peak.

Example 55. The clasp of any of examples 47-49, wherein the curved shape comprises a plurality of peaks arranged between the joint portion and a distal end of the fixed arm and the moveable arm.

Example 56. The clasp of any of examples 47-49, wherein the curved shape comprises a plurality of troughs arranged between the joint portion and a distal end of the fixed arm and the moveable arm.

Example 57. The clasp of any of examples 47-56, further comprising a gripping surface extending along at least a portion of one of the fixed arm and the moveable arm.

Example 58. The clasp of example 57, wherein the gripping surface is formed by powder coating.

Example 59. The clasp of any of examples 57-58, wherein the gripping surface is formed by laser printing.

Example 60. The clasp of any of examples 57-59, wherein the gripping surface comprises a plurality of teeth.

Example 61. The clasp of any of examples 57-60, wherein the gripping surface comprises a plurality of serrations.

Example 62. The clasp of any of examples 57-61, wherein the gripping surface comprises an adhesive coating that releasably adheres to a valve leaflet.

Example 63. The clasp of any of examples 47-62, further comprising a cover that covers at least one of the fixed arm and the moveable arm.

Example 64. The clasp of example 63, wherein the gripping surface comprises a plurality of surface features that extend through the cover.

Example 65. A device comprising: (i) a first collar and a second collar; and (ii) an expandable coaptation element extending between the first collar and the second collar, the expandable coaptation element comprising a middle portion between two end portions, wherein the middle portion is more flexible than the two end portions; wherein moving the second collar in a proximal direction toward the first collar and causes the expandable coaptation element to contract.

Example 66. The device of example 65, wherein the middle portion contracts more than the end portions when the second collar is retracted in the proximal direction.

Example 67. The device of any of examples 65-66, further comprising an actuation member attached to the second collar.

Example 68. The device of any of examples 65-67, further comprising a biasing member extending between the first collar and the second collar, wherein the biasing member biases the second collar away from the first collar in a distal direction.

Example 69. The device of example 68, further comprising an actuation member attached to the second collar, wherein moving the actuation member in the proximal direction compresses the biasing member.

Example 70. The device of any of examples 65-67, further comprising a biasing member extending between the first collar and the second collar, wherein the biasing member biases the second collar toward from the first collar in a distal direction.

Example 71. The device of example 70, further comprising an actuation member attached to the second collar, wherein moving the actuation member in the distal direction extends the biasing member.

Example 72. The device of any of examples 65-71, wherein the expandable coaptation element comprises a tube of braided wires.

Example 73. The device of example 72, wherein braided wires are formed from a shape memory alloy.

Example 74. The device of example 73, wherein the shape memory alloy comprises at least one of nitinol, CuAlNi, NiTi, and another alloy comprising one or more of Zn, Cu, Au, and Fe.

Example 75. The device of any of examples 65-74, wherein the braided wires in the middle portion of the expandable coaptation element are thinner than the braided wires in the end portions.

Example 76. A device comprising: (i) a first nut and a second nut, wherein at least one of the first nut and the second nut comprises a threaded opening; and (ii) a pair of flexible struts extending between the first nut and the second nut, each of the pair of flexible struts comprising a first threaded end, a second threaded end, and a flexible middle portion, wherein the first threaded end of each flexible strut engages the threaded opening of the first nut and the second threaded end of each flexible struts engages the threaded opening of the second nut; wherein rotation of the first nut relative to the first threaded ends of the pair of flexible struts moves the first threaded ends towards and away from the first nut; wherein rotation of the second nut relative to the second threaded ends of the pair of flexible struts moves the second threaded ends towards and away from the second nut; and wherein moving at least one of the first threaded ends away from the first nut and the second threaded ends away from the second nut causes the flexible middle portion of each flexible struts to expand laterally outward.

Example 77. The device of example 76, wherein at least one of the first nut and the second nut includes an engagement portion; and wherein moving the respective one of the first threaded ends and the second threaded ends towards the engagement portion causes the engagement portion to contact the flexible middle portion and move the flexible middle portion laterally inward.

Example 78. The device of any of example 76 and example 77, wherein the threaded openings of the first nut and the second nut comprise opposite-handed threads.

Example 79. The device of any of examples 76-78, wherein the threaded openings of the first nut and the second nut comprise opposite-handed threads.

Example 80. The device of any of examples 76-78, wherein only one of the first nut and the second nut are rotated to expand and contract the flexible middle portion of the flexible struts.

Example 81. A device comprising: (i) a first actuation member moveably attached to a second actuation member, wherein engaging at least one of the first actuation member and the second actuation member moves the first actuation member and the second actuation member toward each other; and (ii) an expandable coaptation element, wherein the expandable coaptation element comprises an expansion member and a spreader member that are both arranged between the first and the second actuation members, wherein movement of the first and the second actuation member towards each other causes the spreader member to engage and expand the expansion member.

Example 82. The device of example 81, wherein the first actuation member comprises a distally extending threaded rod; wherein the distally extending threaded rod extends through the expandable coaptation element; and wherein the second actuation member comprise a threaded opening for engaging the distally extending threaded rod of the first actuation member.

Example 83. The device of any of example 81 and example 82, wherein the expansion member comprises two expandable rings arranged between the spreader member and each of the first second member and the second actuation member.

Example 84. The device of any of examples 81-83, wherein the spreader member comprises a tapered end for engaging the expansion member.

Example 85. The device of example 84, wherein the expansion member comprises a chamfered inner diameter for engaging the tapered end of the expansion member.

Example 86. The device of any of examples 81-85, further comprising a threaded actuation member comprising a first threaded portion and a second threaded portion, wherein the first threaded portion and the second threaded portion have opposite-handed threads; wherein the first actuation member comprises a threaded opening for engaging the first threaded portion of the threaded actuation member; wherein the second actuation member comprises a threaded opening for engaging the second threaded portion of the threaded actuation member; and wherein rotation of the threaded actuation member moves the first and the second actuation members towards and away from each other.

Example 87. The device of any of examples 81-86, further comprising a cover that covers the spreader member and the expansion member.

Example 88. A device comprising: (i) a fixed actuation member; (ii) a moveable actuation member; (iii) a threaded shaft extending through the moveable actuation member and rotatably attached to the first actuation member; and (iv) a plurality of struts extending between the fixed actuation member and the moveable actuation member, each strut comprising a plurality of rigid portions connected together by a plurality of hinge portions; wherein rotating the threaded shaft causes the moveable actuation member to move towards and away from the fixed actuation member to cause the plurality of struts to expand and contract, respectively.

Example 89. The device of example 88, wherein each of the plurality of struts comprises three rigid portions.

Example 90. The device of any of example 88 and example 89, wherein a second rigid portion of the three rigid portions comprises a mounting location.

Example 91. The device of any of examples 88-90, wherein the plurality of struts comprises four struts.

Example 92. The device of example 91, wherein the four struts are arranged in two pairs of opposing struts and each strut of one pair of opposing struts comprises a mounting location in one of the rigid portions of the strut.

Example 93. The device of example 91, wherein the four struts are arranged in two pairs of opposing struts and one pair of opposing struts expands at a different rate than the other pair of opposing struts.

Example 94. A device comprising: (i) a first actuation member; (ii) a second actuation member; (iii) a threaded shaft comprising a first threaded portion for engaging the first actuation member and a second threaded portion for engaging the second actuation member, wherein the first threaded portion and the second threaded portion comprise opposite-handed threads; and (iv) a plurality of struts extending between the first actuation member and the second actuation member, each strut comprising a plurality of rigid portions connected together by a plurality of hinge portions; wherein rotating the threaded shaft causes the first actuation member and the second actuation member to move towards and away from each other to cause the plurality of struts to expand and contract, respectively.

Example 95. The device of example 94, wherein each of the plurality of struts comprises three rigid portions.

Example 96. The device of example 95, wherein a second rigid portion of the three rigid portions comprises a mounting location.

Example 97. The device of any of examples 94-96, wherein the plurality of struts comprises four struts.

Example 98. The device of example 97, wherein the four struts are arranged in two pairs of opposing struts and each strut of one pair of opposing struts comprises a mounting location in one of the rigid portions of the strut.

Example 99. The device of example 97, wherein the four struts are arranged in two pairs of opposing struts and one pair of opposing struts expands at a different rate than the other pair of opposing struts.

Example 100. An device comprising: (i) an outer tube attached to a fixed actuation member, the outer tube comprising a plurality of openings; (ii) a latch tube attached to a moveable actuation member, the latch tube comprising a latch member; (iii) a securing member that extends through the latch tube, the securing member having a tapered distal end; and (iv) a plurality of struts extending between the fixed actuation member and the moveable actuation member, each strut comprising a plurality of rigid portions connected together by a plurality of hinge portions; wherein when the securing member is inserted into the latch tube, the tapered distal end engages the latch member and retains the latch member in an unlatched condition so that the latch tube can be moved to a desired position in the outer tube; wherein the securing member can be withdrawn from the latch tube to disengage the securing member from the latch member to facilitate movement of the latch member to the latched condition wherein the latch member engages one of the plurality of openings of the outer tube; and wherein extending and retracting the latch tube causes the moveable actuation member to move towards and away from the fixed actuation member to cause the plurality of struts to expand and contract, respectively.

Example 101. The device of example 100, wherein the latch member is laser cut from the latch tube.

Example 102. The device of any of example 100 and example 101, wherein the latch tube and latch member are formed from a shape memory alloy.

Example 103. The device of example 102, wherein the latch member is shape set in the latched condition.

Example 104. The device any of examples 100-103, wherein each of the struts comprises two rigid portions joined by a hinge portion.

Example 105. The device of example 104, wherein a pair of the plurality of struts are connected at proximal and distal ends.

Example 106. The device of example 105, wherein the pair of connected struts are cut from a sheet of material.

Example 107. The device of any of examples 105-106, wherein two pairs of connected struts are connected to each other by a side plate.

Example 108. The device of example 107, wherein the side plate comprises a mounting portion.

Example 109. A device comprising: (i) an actuation spool; (ii) a central frame extending from the actuation spool; (iii) a pivoting frame pivotably attached to the central frame, wherein the pivoting frame has a round shape; and (iv) an actuation member extending from the actuation spool to the pivoting frame, wherein rotation of the actuation spool retracts and releases the actuation member to cause the pivoting frame to pivot.

Example 110. The device of example 109, further comprising a plurality of pivoting frames, wherein at least one pivoting frame is smaller than the other of the plurality of pivoting frames.

Example 111. The device of any of example 109 and example 110, wherein at least pivoting frame is shaped to fill a gap between native leaflets.

Example 112. The device of any of examples 109-111, further comprising a plurality of pivoting frames, wherein at least one actuation element connects a first pivoting frame to a second pivoting frame so that pivoting the first pivoting frame causes the second pivoting frame to pivot.

Example 113. The device of any of examples 109-112, wherein when the actuation spool is rotated a first half of the pivoting frame moves toward the actuation spool and a second half of the pivoting frame moves away from the actuation spool.

Example 114. The device of any of examples 109-112, wherein when the actuation spool is rotated a first half of the pivoting frame moves toward the actuation spool and a second half of the pivoting frame moves toward from the actuation spool.

Example 115. The device of any of examples 109-114, wherein the pivoting frame extends through the central frame.

Example 116. The device of any of examples 109-114, wherein the pivoting frame extends to the central frame.

Example 117. The device of any of examples 109-116, wherein the actuation member is released when the actuation spool is rotated in a first direction and the actuation member is retracted when the spool is rotated in a second direction.

Example 118. The device of any of examples 109-116, wherein a first actuation member is released and a second actuation member is retracted when the actuation spool is rotated in a first direction and the first actuation member is retracted and the second actuation member is released when the actuation spool is rotated in a second direction.

Example 119. The device of any of examples 109-116, wherein a first actuation member is released and a second actuation member is released when the actuation spool is rotated in a first direction and the first actuation member is retracted and the second actuation member is retracted when the actuation spool is rotated in a second direction.

Example 120. A heart valve repair system comprising: (A) a device comprising a proximal collar, the proximal collar comprising a pair of protrusions extending radially inward; (B) a delivery system comprising: (i) a distal coupler comprising a pair of moveable arms moveable between an engaged position and a disengaged position, the moveable arms each comprising an opening for engaging one of the pair of protrusions of the proximal collar; (ii) an actuation member extending through the distal coupler between the pair of moveable arms, wherein the actuation member retains the pair of moveable arms in an engaged position; wherein retracting the actuation member through the distal coupler allows the pair of moveable arms to disengage from the pair of protrusions.

Example 121. The heart valve repair system of example 120, wherein the moveable arms are biased in an inward direction.

Example 122. The heart valve repair system of example 120 or example 121, wherein the moveable arms comprise engagement portions for engaging the actuation member.

Example 123. The heart valve repair system of example 122, wherein the engagement portions are compressed between the actuation member and the moveable arms so that the moveable arms are pressed against the pair of protrusions of the proximal collar.

Example 124. The heart valve repair system of example 122 or example 123, wherein the engagement portions have a rounded shape.

Example 125. A device comprising: (i) an outer tube comprising an opening; (ii) an inner tube arranged concentrically within the outer tube; (iii) one or more expandable coaptation members extending from a first end to a second end, wherein the first end is hingeably attached to the outer tube and the second end is hingeably attached to the inner tube through the opening in the outer tube; wherein rotating the outer tube relative to the inner tube causes the expandable coaptation member to expand radially outward.

Example 126. The device of example 125, wherein the one or more expandable coaptation members comprises a plurality of links connected together by a plurality of hinge portions.

Example 127. The device of any of example 125 and example 126, wherein the one or more expandable coaptation members are cut from the outer tube.

Example 128. The device of any of examples 125-127, wherein the device comprises three expandable coaptation members that are radially spaced apart from each other equally.

Example 129. The device of any of examples 125-128, wherein the hinge portions comprise a plurality of cuts.

Example 130. The device of any of examples 125-129, wherein the one or more expandable coaptation members vary in width between the first end and the second end.

Example 131. The device of any of examples 125-130, wherein the one or more expandable coaptation members are formed from a shape memory alloy.

Example 132. The device of example 131, wherein the one or more expandable coaptation members are biased in the expansion direction.

Example 133. A heart valve repair system comprising: (i) a device comprising a coaptation portion and an anchor portion, the coaptation portion comprising an outer tube comprising an opening, an inner tube arranged concentrically within the outer tube, one or more expandable coaptation members extending from a first end to a second end, wherein the first end is hingeably attached to the outer tube and the second end is hingeably attached to the inner tube through the opening in the outer tube; and (ii) a delivery system comprising a delivery sheath and an actuation member, wherein the actuation member engages one of the inner tube and the outer tube such that rotating the actuation member causes the one or more expandable coaptation members to expand radially outward.

Example 134. The heart valve repair system of example 133, wherein the expandable coaptation member are biased in an expansion direction.

Example 135. The heart valve repair system of example 133 or example 134, wherein the device further comprises a cover that covers the one or more expandable coaptation member.

Example 136. A device comprising: (i) a flexible enclosure forming an expandable cavity, wherein the flexible enclosure is expandable from an unexpanded to an expanded condition; (ii) an elongated filling element filling at least a portion of the expandable cavity when the flexible enclosure is in the expanded condition, wherein the elongated filling element provides resistance to compression of the flexible enclosure in the expanded condition.

Example 137. The device of example 136, wherein the flexible enclosure is formed from a woven textile material.

Example 138. The device of example 136, wherein the flexible enclosure is formed from a molded polymer material.

Example 139. The device of any of examples 136-138, wherein the elongated filling element comprises a metal coil.

Example 140. The device of any of examples 136-138, wherein the elongated filling element comprises a polymer coil.

Example 141. The device of any of examples 136-140, wherein the elongated filling element comprises a radiopaque material.

Example 142. The device of example 141, wherein the radiopaque material is formed into a plurality of markers attached to the elongated filling element.

Example 143. The device of any of examples 135-142, further comprising a second elongated filling element disposed within the expandable cavity.

Example 144. The device of example 143, wherein the second elongated filling element is formed from a material that is different from the elongated filling element.

Example 145. The device of any of examples 135-144, further comprising a plurality of pieces of filling material.

Example 146. The device of any of examples 135-145, wherein at least one of the elongated filling element, the second elongated filling element, and the plurality of pieces of filling material are coated with a hydrogel coating.

Example 147. The device of any of examples 135-146, wherein the flexible enclosure comprises an expandable frame formed from a shape memory alloy.

Example 148. A heart valve repair system comprising: (i) a device comprising a coaptation portion and an anchor portion, the coaptation portion comprising a flexible enclosure that is expandable from an unexpanded to expanded condition and that forms an expandable cavity, and an elongated filling element filling at least a portion of the expandable cavity when the flexible enclosure is in the expanded condition, wherein the elongated filling element provides resistance to compression of the flexible enclosure in the expanded condition; and (ii) a delivery system comprising a delivery sheath and an actuation member, wherein the actuation member facilitates the expansion of the flexible enclosure, and the elongated filling element is delivered into the expandable cavity via the delivery sheath.

Example 149. The heart valve repair system of example 148, wherein the actuation member comprises a tube for filling the expandable cavity with saline to cause the flexible enclosure to expand.

Example 150. The heart valve repair system of example 148, wherein the flexible enclosure is caused to expand by delivery of the elongated filling element.

The above method(s) can be performed on a living animal or on a simulation, such as on a cadaver, cadaver heart, anthropomorphic ghost, simulator (e.g., with the body parts, heart, tissue, etc. being simulated), etc.

Any of the various systems, devices, apparatuses, etc. in this disclosure (including in any of the examples above) can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—can be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts can be expressly identified herein as being inventive or forming part of a disclosure, such identification is not intended to be exclusive, but rather there can be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the treatment techniques, methods, operations, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated). The words used in the claims have their full ordinary meanings and are not limited in any way by the description of the examples in the specification.

What is claimed is:

1. An actuation mechanism for an implantable device, the actuation mechanism comprising:
an outer tube comprising a plurality of openings or recesses;
a latch tube attached to an adjustable member, the latch tube comprising a latch member and an opening;
an actuation member having a threaded portion that can be threadably attached to the opening in the latch tube and a having tapered distal end;
wherein when the threaded portion of the actuation member is threaded into the opening of the latch tube, the tapered distal end engages the latch member and retains the latch member in an unlatched condition so that the latch tube can be moved to a desired position in the outer tube; and
wherein the actuation member can be unthreaded from the opening of the latch tube to disengage the tapered distal end from the latch member to facilitate movement of the latch member to a latched condition wherein the latch member engages one of the plurality of openings or recesses of the outer tube.

2. The actuation mechanism of claim 1, wherein the latch member is laser cut from the latch tube.

3. The actuation mechanism of claim 1, wherein the latch member is biased toward the latched condition.

4. The actuation mechanism of claim 1, wherein the latch tube further comprises an orientation tab that extends through an orientation slot of the outer tube to prohibit relative rotation of the latch tube and the outer tube.

5. The actuation mechanism of claim 1 wherein the actuation mechanism is controlled by a handle that includes a handle release assembly, the handle release assembly comprising:
an actuation element extending from a distal end for engaging the implantable device to a proximal end that is secured to an actuation element adaptor;
a width adjustment element extending within the actuation element from a distal end for engaging the implantable device to a proximal end, the proximal end secured to a width adjustment element adaptor;
a connector body having a central lumen through which the actuation element and the width adjustment element extend, the connector body comprising a coupling portion that includes a retention groove;
an inner body comprising attachment members for engaging the coupling portion and the retention groove of the connector body, a recess for receiving the actuation element adaptor, a catch, and side slots;
an outer body comprising a proximal stop, a latch arm having a protrusion for engaging the catch of the inner body to prohibit relative movement of the outer body and the inner body, and side slots, wherein the width adjustment element adaptor extends through the side slots of the inner body and the side slots of the outer body;
a gripping portion slidably attached to the outer body;
wherein sliding the gripping portion in a proximal direction exposes the latch arm of the outer body to permit the latch arm to disengage from the catch of the inner body;
wherein after disengaging the latch arm, the outer body is moved proximally by application of further proximal force on the gripping portion to move the outer body proximally so that the side slots engage the width adjustment element adaptor to move the width adjustment element in the proximal direction; and
wherein further proximal movement of the gripping portion and the outer body exposes the attachment members of the inner body and causes the width adjustment element adaptor to engage the side slots of the inner body to disengage the attachment members from the retention groove of the coupling portion of the connector body and to pull the actuation element in the proximal direction.

6. A heart valve repair system comprising:
a device comprising a proximal collar, the proximal collar comprising a pair of protrusions extending radially inward;
a delivery system comprising:
a distal coupler comprising a pair of moveable arms moveable between an engaged position and a disengaged position, the pair of moveable arms each comprising an opening for engaging one of the pair of protrusions of the proximal collar;
an actuation member extending through the distal coupler between the pair of moveable arms, wherein the actuation member retains the pair of moveable arms in the engaged position; and
wherein retracting the actuation member through the distal coupler allows the pair of moveable arms to disengage from the pair of protrusions.

7. The heart valve repair system of claim 6, wherein the pair of moveable arms are biased in an inward direction.

8. The heart valve repair system of claim 6, wherein the pair of moveable arms comprise engagement portions for engaging the actuation member.

9. The heart valve repair system of claim 8, wherein the engagement portions are compressed between the actuation member and the pair of moveable arms so that the pair of moveable arms are pressed against the pair of protrusions of the proximal collar.

10. The heart valve repair system of claim 8, wherein the engagement portions have a rounded shape.

11. The heart valve repair system of claim 6, wherein the device includes a clasp comprising:
a fixed arm having a curved shape;
a moveable arm having a curved shape that is complementary to the curved shape of the fixed arm; and
a joint portion that hingeably connects the moveable arm to the fixed arm.

12. The heart valve repair system of claim 6, wherein the device comprises:
a first nut and a second nut, wherein at least one of the first nut and the second nut comprises a threaded opening;
a pair of flexible struts extending between the first nut and the second nut, each of the pair of flexible struts comprising a first threaded end, a second threaded end, and a flexible middle portion, wherein the first threaded end of each flexible strut engages the threaded opening of the first nut and the second threaded end of each flexible struts engages the threaded opening of the second nut;
wherein rotation of the first nut relative to the first threaded ends of the pair of flexible struts moves the first threaded ends towards and away from the first nut;
wherein rotation of the second nut relative to second threaded ends of the pair of flexible struts moves the second threaded ends towards and away from the second nut; and
wherein moving at least one of the first threaded ends away from the first nut and the second threaded ends away from the second nut causes the flexible middle portion of each flexible strut to expand laterally outward.

13. The heart valve repair system of claim 6, wherein the device comprises:
an outer tube comprising an opening;
an inner tube arranged concentrically within the outer tube;
one or more expandable coaptation members extending from a first end to a second end, wherein the first end is hingeably attached to the outer tube and the second end is hingeably attached to the inner tube through the opening in the outer tube; and
wherein rotating the outer tube relative to the inner tube causes the one or more expandable coaptation members to expand radially outward.

14. The heart valve repair system of claim 6 wherein the device comprises:
a flexible enclosure forming an expandable cavity, wherein the flexible enclosure is expandable from an unexpanded to an expanded condition; and
an elongated filling element filling at least a portion of the expandable cavity when the flexible enclosure is in the expanded condition, wherein the elongated filling element provides resistance to compression of the flexible enclosure in the expanded condition.

15. An implantable device comprising:
a fixed actuation member;
a moveable actuation member;
a threaded shaft extending through the moveable actuation member and rotatably attached to the fixed actuation member;
a plurality of struts extending between the fixed actuation member and the moveable actuation member, each strut comprising a plurality of rigid portions connected together by a plurality of hinge portions; and
wherein rotating the threaded shaft causes the moveable actuation member to move towards and away from the fixed actuation member to cause the plurality of struts to expand and contract, respectively.

16. The implantable device of claim 15, wherein each of the plurality of struts comprises three rigid portions.

17. The implantable device of claim 16, wherein a second rigid portion of the three rigid portions comprises a mounting location.

18. The implantable device of claim 15, wherein the plurality of struts comprises four struts.

19. The implantable device of claim 18, wherein the four struts are arranged in two pairs of opposing struts and each strut of one pair of opposing struts comprises a mounting location in one of the rigid portions of the strut.

20. The implantable device of claim 18, wherein the four struts are arranged in two pairs of opposing struts and one pair of opposing struts expands at a different rate than the other pair of opposing struts.
